SEQUENCE LISTING (15 sequences)

SEQ ID NO: 1 - protein, 340 aa, Sulfolobus solfataricus
MIVLFVDFDYFYAQVEEVLNPSLKGKPVVVCVFSGRFEDSGVVATANYEARKFGVYAGIPIVEAKKILPNAVYLPWRDLVYWGVSERIMNLLREYSEKIEIASIDEAYLDISDKVRDYREAYNLGLEIKNKILEKEKITVTVGISKNKVFAAVAGRMAKPNGIKVIDDEEVKRLIRELDIADVQGIPYFTAEKLKKLGINKLVDTLSIEFDKLKGMIGEAKAKYLISLARDEYNEPIRTRVRKSIGRTVTMKRNSRNLEEIKPYLFRAIEESYYKLDKRIPKAIHVVAWKSYWNSQYRWSWFPHGISKETAYSESVQLLQQILKKDKRKIRRIGVRFSKF

SEQ ID NO: 2 - protein, 340 aa, Sulfolobus solfataricus
MIVLFVDFDYFYAQVEEVLNPSLKGKPVVVCVFSGRFEDSGVVATANYEARKFGVYAGIPIVRAKKILPNAVYLPWRDLVYWGVSERIMNLLREYSEKIEIASIDEAYLDISDKVRDYREAYNLGLEIKNKILEKEKITVTVGISKNKVFAAVAGRMAKPNGIKVIDDEEVKRLIRELDIADVQGIPYFTAEKLKKLGINKLVDTLSIEFDKLKGMIGEAKAKYLISLARDEYNEPIRTRVRKSIGRTVTMKRNSRNLEEIKPYLFRAIEESYYKLDKRIPKAIHVVAWKSYWNSQYRWSWFPHGISKETAYSESVQLLQQILKKDKRKIRRIGVRFSKF

SEQ ID NO: 3 - protein, 340 aa, Sulfolobus solfataricus
MIVLFVDFDYFYAQVEEVLNPSLKGKPVVVCVFSGRFEDSGVVATANYEARKFGVYAGIPIVRAKKILPNAVYLPWRDLVYWGVSERIMNLLREYSEKIEIASIDEAYLDISDKVRDYREAYNLGLEIKNKILEKEKITVTVGISKNKVFAAVAGRMAKPNGIKVIDDEEVKRLIRELDIADVQGIPYFTAEKLKKLGINKLVDTLSIEFDKLKGMIGEAKAKYLISLARDEYNEPIRTRVRRSIGRTVTMKRNSRNLEEIKPYLFRAIEESYYKLDKRIPKAIHVVAWKSYWNSQYRWSWFPHGISKETAYSESVQLLQQILKKDKRKIRRIGVRFSKF

SEQ ID NO: 4 - protein, 340 aa, Sulfolobus solfataricus
MIVLFVDFDYFYAQVEEVLNPSLKGKPVVVCVFSGRFEDSGVVATANYEARKFGVYAGIPIVRAKKILPNAVYLPWRDLVYWGVSERIMNLLREYSEKIEIASIDEAYLDISDKVRDYREAYNLGLEIKNKILEKEKITVTVGISKNKVFAAVAGRMAKPNGIKVIDDEEVKRLIRELDIADVQGIPYFTAEKLKKLGINKLVDTLSIEFDKLKGMIGEAKAKYLISLARDEYNEPIRTRVRKSIGRTVTMKRDSRNLEEIKPYLFRAIEESYYKLDKRIPKAIHVVAWKSYWNSQYRWSWFPHGISKETAYSESVQLLQQILKKDKRKIRRIGVRFSKF

SEQ ID NO: 5 - protein, 340 aa, Sulfolobus solfataricus
MIVLFVDFDYFYAQVEEVLNPSLKGKPVVVCVFSGRFEDSGVVATANYEARKFGVYAGIPIKRAKKILPNAVYLPWRDLVYWGVSERIMNLLREYSEKIEIASIDEAYLDISDKVRDYREAYNLGLEIKNKILEKEKITVTVGISKNKVFAAVAGRMAKPNGIKVIDDEEVKRLIRELDIADVQGIPYFTAEKLKKLGINKLVDTLSIEFDKLKGMIGEAKAKYLISLARDEYNEPIRTRVRKSIGRTVTMKRNSRNLEEIKPYLFRAIEESYYKLDKRIPKAIHVVAWKSYWNSQYRWSWFPHGISKETAYSESVQLLQQILKKDKRKIRRIGVRFSKF

SEQ ID NO: 6 - protein, 356 aa, Thermococcus kodakarensis
MEVLTKDEIINRIIRERGLSRSEIEEKIRELAKMHGVSENAAAVMLAEELGVSLGKEEEMLYIKDLVPGMTGVNIVARIKRKFPPREYTRRDGSTGRVADLIIYDSTGQARLVLWDAMVAKYYDDLNVGDVIKVIDPTVKEGMRGVELHANFRTRIIKNPEDPRVEEIPPLEEVRSYNYRRVQIKELQGGERFVEVRGTIAKLYRVLVYDACPECRRRVDYDPSTDTWICPEHGPVNPVKITVLDFGLDDSTGYIRTTLFGDSAAELIGEEPEVIDEKLKKLIDEGLTPKEAGKRLAEDEYYPLIGKEIVVRGSVVEDKFLGTLFKARSWDEVNEKAEIERVRRELYRELKEYGLE

SEQ ID NO: 7 - protein, 301 aa, Bacteriophage T4
MFKRKSTAELAAQMAKLNGNKGFSSEDKGEWKLKLDNAGNGQAVIRFLPSKNDEQAPFAILVNHGFKKNGKWYIETCSSTHGDYDSCPVCQYISKNDLYNTDNKEYSLVKRKTSYWANILVVKDPAAPENEGKVFKYRFGKKIWDKINAMIAVDVEMGETPVDVTCPWEGANFVLKVKQVSGFSNYDESKFLNQSAIPNIDDESFQKELFEQMVDLSEMTSKDKFKSFEELNTKFGQVMGTAVMGGAAATAAKKADKVADDLDAFNVDDFNTKTEDDFMSSSSGSSSSADDTDLDDLLNDL

SEQ ID NO: 8 - protein, 263 aa, Thermus thermophilus
MARGLNRVFLIGALATRPDMRYTPAGLAILDLTLAGQDLLLSDNGGEREVSWYHRVRLLGRQAEMWGDLLDQGQLVFVEGRLEYRQWEREGEKRSELQIRADFLDPLDDRGKERAEDSRGQPRLRAALNQVFLMGNLTRDPELRYTPQGTAVARLGLAVNERRQGAEERTHFVEVQAWRDLAEWAAELRKGDGLFVIGRLVNDSWTSSSGERRFQTRVEALRLERPTRGPAQAGGSRSREVQTGGVDIDEGLEDFPPEEELPF

SEQ ID NO: 9 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQSGWGQPQQPQGGNQFSGGAQSRPQQSAPAAPSNEPPMDFDDDIPF

SEQ ID NO: 10 - protein, 353 aa, Escherichia coli
MAIDENKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYARKLGVDIDNLLCSQPDTGEQALEICDALARSGAVDVIVVDSVAALTPKAEIEGEIGDSHMGLAARMMSQAMRKLAGNLKQSNTLLIFINQIRMKIGVMFGNPETTTGGNALKFYASVRLDIRRIGAVKEGENVVGSETRVKVVKNKIAAPFKQAEFQILYGEGINFYGELVDLGVKEKLIEKAGAWYSYKGEKIGQGKANATAWLKDNPETAKEIEKKVRELLLSNPNSTPDFSVDDSEGVAETNEDF

SEQ ID NO: 11 - protein, 616 aa, Homo sapiens
MVGQLSEGAIAAIMQKGDTNIKPILQVINIRPITTGNSPPRYRLLMSDGLNTLSSFMLATQLNPLVEEEQLSSNCVCQIHRFIVNTLKDGRRVVILMELEVLKSAEAVGVKIGNPVPYNEGLGQPQVAPPAPAASPAASSRPQPQNGSSGMGSTVSKAYGASKTFGKAAGPSLSHTSGGTQSKVVPIASLTPYQSKWTICARVTNKSQIRTWSNSRGEGKLFSLELVDESGEIRATAFNEQVDKFFPLIEVNKVYYFSKGTLKIANKQFTAVKNDYEMTFNNETSVMPCEDDHHLPTVQFDFTGIDDLENKSKDSLVDIIGICKSYEDATKITVRSNNREVAKRNIYLMDTSGKVVTATLWGEDADKFDGSRQPVLAIKGARVSDFGGRSLSVLSSSTIIANPDIPEAYKLRGWFDAEGQALDGVSISDLKSGGVGGSNTNWKTLYEVKSENLGQGDKPDYFSSVATVVYLRKENCMYQACPTQDCNKKVIDQQNGLYRCEKCDTEFPNFKYRMILSVNIADFQENQWVTCFQESAEAILGQNAAYLGELKDKNEQAFEEVFQNANFRSFIFRVRVKVETYNDESRIKATVMDVKPVDYREYGRRLVMSIRRSALM

SEQ ID NO: 12 - protein, 71 aa, HIV-1
MQRGNFRNQRKMVKCFNCGKEGHTARNCRAPRKKGCWKCGKEGHQMKDCTERQANFLGKIWPSYKGRPGNF

SEQ ID NO: 13 - DNA, 44 bp, synthetic construct
  source 1..44: mol_type = other DNA
ctttgtgtgacccactggtccagatagatgccttttcctttttt

SEQ ID NO: 14 - DNA, 59 bp, synthetic construct
  source 1..59: mol_type = other DNA
taccccagacctgaagctctcttctggtggggctgttggctctggtctgctctgaagaa

SEQ ID NO: 15 - DNA, 79 bp, synthetic construct
  source 1..79: mol_type = other DNA
taccccagacctgaagctctcttctggtggggctgttggctctggtctgctctgaagaaaattccctggccttcccttg

(19) United States
(12) Patent Application Publication
(10) Pub. No.: US 2026/0365633 A1
(43) Pub. Date: Nov. 19, 2026

(54) Novel PEM Structures for SBX

(71) Applicant: Roche Sequencing Solutions, Inc., Pleasanton, CA (US)

(72) Inventors: Brent Banasik, Seattle, WA (US); Kendall Marie Berg, Seattle, WA (US); Ali Marie Carroll, Seattle, WA (US); Jagadeeswaran Chandrasekar, Seattle, WA (US); Ajay Kumar Chinnam, Seattle, WA (US); Peter Crisalli, Santa Clara, CA (US); Abdul Rouf Dar, Fremont, CA (US); Grant Christopher Derdeyn-Blackwell, Seattle, WA (US); Grisha Etkin, Cupertino, CA (US); Aaron Jacobs, Seattle, WA (US); Omid Khakshoor, Lathrop, CA (US); Mark Stamatios Kokoris, Bothell, WA (US); Melud Nabavi, Seattle, WA (US); Dylan O'Connell, Seattle, WA (US); Tavis William Price, Seattle, WA (US); Cameron John Szczesny, Seattle, WA (US); Meng C. Taing, Hayward, CA (US)

(21) Appl. No.: 19/704,039

(22) Filed: Jun. 10, 2026

Related U.S. Application Data
(63) Continuation of application No. PCT/US2024/061051, filed on Dec. 19, 2024.
(60) Provisional application No. 63/614,218, filed on Dec. 22, 2023.
(60) Provisional application No. 63/614,144, filed on Dec. 22, 2023.
(60) Provisional application No. 63/680,104, filed on Aug. 7, 2024.
(60) Provisional application No. 63/687,453, filed on Aug. 27, 2024.
(60) Provisional application No. 63/688,911, filed on Aug. 30, 2024.

(51) Int. Cl. C07D 401/14; C07D 213/77; C07D 249/04; C07D 249/06; C07D 307/66; C07D 401/04; C07D 403/14; C07D 405/14; C07D 409/14; C07D 421/14; C07D 471/04; C07D 473/00; C07D 519/00

(52) U.S. Cl. CPC C07D 401/14; C07D 213/77; C07D 249/04; C07D 249/06; C07D 307/66; C07D 401/04; C07D 403/14; C07D 405/14; C07D 409/14; C07D 421/14; C07D 471/04; C07D 473/00; C07D 519/00

(57) ABSTRACT

The present disclosure pertains to compounds, methods and compositions for improving on nucleic acid polymerization, including DNA replication by in vitro primer extension to generate, for example, polymers for nanopore-based single molecule sequencing of a DNA template. A nucleic acid polymerase reaction composition is provided with polymerization enhancement moieties, which allows enhanced DNA polymerase activity with nucleotide analogs, resulting in improved length of primer extension products for sequencing applications.

### SEQUENCE LISTING

The contents of the electronic sequence listing (P39808-US-1_Sequence_Listing.xml; Size: 18,432 bytes; and Date of Creation: Jun. 9, 2026) is herein incorporated by reference in its entirety.

### CROSS REFERENCE TO RELATED APPLICATIONS

This patent application is a continuation of International Patent Application No. PCT/US2024/061,051, filed Dec. 19, 2024, which claims priority to and the benefit of U.S. Provisional Application No. 63/614,144, filed Dec. 22, 2023, U.S. Provisional Application No. 63/680,104, filed Aug. 7, 2024, United States Provisional Application No. 63,614,218, filed Dec. 22, 2023, U.S. Provisional Application No. 63/687,453, filed Aug. 27, 2024 and U.S. Provisional Application No. 63/688,911, filed Aug. 30, 2024. Each of the above patent applications is incorporated herein by reference as if set forth in its entirety.

### FIELD OF THE DISCLOSURE

The present disclosure relates to new chemical entities, more specifically to new organic molecules optionally having inorganic components, including compositions thereof, and methods for the manufacture and utilization thereof, particularly in influencing enzyme performance.

### BACKGROUND

Measurement of biomolecules is a foundation of modern medicine and is broadly used in medical research, and more specifically in diagnostics and therapy, as well in drug development. Nucleic acids encode the necessary information for living things to function and reproduce and are essentially a blueprint for life. Determining such blueprints is useful in pure research as well as in applied sciences. In medicine, sequencing can be used for diagnosis and to develop treatments for a variety of pathologies, including cancer, heart disease, autoimmune disorders, multiple sclerosis, and obesity. In industry, sequencing can be used to design improved enzymatic processes or synthetic organisms. In biology, this tool can be used to study the health of ecosystems, for example, and thus have a broad range of utility. Similarly, measurement of proteins and other biomolecules has provided markers and understanding of disease and pathogenic propagation.

An individual's unique DNA sequence provides valuable information concerning their susceptibility to certain diseases. It also provides patients with the opportunity to screen for early detection and/or to receive preventative treatment. Furthermore, given a patient's individual blueprint, clinicians will be able to administer personalized therapy to maximize drug efficacy and/or to minimize the risk of an adverse drug response. Similarly, determining the blueprint of pathogenic organisms can lead to new treatments for infectious diseases and more robust pathogen surveillance. Low cost, whole genome DNA sequencing will provide the foundation for modern medicine. To achieve this goal, sequencing technologies must continue to advance with respect to throughput, accuracy, and read length.

Over the last decade, a multitude of next generation DNA sequencing technologies have become commercially available and have dramatically reduced the cost of sequencing whole genomes. These include sequencing by synthesis (“SBS”) platforms (Illumina, Inc., 454 Life Sciences, Ion Torrent, Pacific Biosciences) and analogous ligation-based platforms (Complete Genomics, Life Technologies Corporation). A number of other technologies are being developed that utilize a wide variety of sample processing and detection methods. For example, GnuBio, Inc. (Cambridge, Mass.) uses picoliter reaction vessels to control millions of discreet probe sequencing reactions, whereas Halcyon Molecular (Redwood City, Calif.) was attempting to develop technology for direct DNA measurement using a transmission electron microscope.

Nanopore based nucleic acid sequencing is a compelling approach that has been widely studied. Kasianowicz et al. (Proc. Natl. Acad. Sci. USA 93: 13770-13773, 1996) characterized single-stranded polynucleotides as they were electrically translocated through an alpha hemolysin nanopore embedded in a lipid bilayer. It was demonstrated that during polynucleotide translocation partial blockage of the nanopore aperture could be measured as a decrease in ionic current. Polynucleotide sequencing in nanopores, however, is burdened by having to resolve tightly spaced bases (0.34 nm) with small signal differences immersed in significant background noise. The measurement challenge of single base resolution in a nanopore is made more demanding due to the rapid translocation rates observed for polynucleotides, which are typically on the order of 1 base per microsecond. Translocation speed can be reduced by adjusting run parameters such as voltage, salt composition, pH, temperature, and viscosity, to name a few. However, such adjustments have been unable to reduce translocation speed to a level that allows for single base resolution.

Stratos Genomics has developed a method called Sequencing by Expansion (“SBX”) that uses a biochemical process to transcribe the sequence of DNA onto a measurable polymer called an “Xpandomer” (Kokoris et al., U.S. Pat. No. 7,939,259, “High Throughput Nucleic Acid Sequencing by Expansion”). The transcribed sequence is encoded along the Xpandomer backbone in high signal-to-noise reporters that are separated by about 10 nm and are designed for high-signal-to-noise, well-differentiated responses. These differences provide significant performance enhancements in sequence read efficiency and accuracy of Xpandomers relative to native DNA. Xpandomers can enable several next generation DNA sequencing detection technologies and are well suited to nanopore sequencing.

Xpandomers are generated from non-natural nucleotide analogs, termed XNTPs, characterized by lengthy substituents that enable the Xpandomer backbone to be expanded following synthesis (see Published PCT Appl. No. WO2016/081871 to Kokoris et al., herein incorporated by reference in its entirety). Because of their atypical structures, XNTPs, as well as other nucleotide analogs (e.g., nucleotide analogs modified with detectable label moieties) introduce novel challenges as substrates for currently available DNA polymerases. Published PCT Appl. Nos. WO2017/087281 and WO2018/204717 to Kokoris et al., herein incorporated by reference in their entirety, describes engineered DP04 polymerase variants with enhanced primer extension activity utilizing non-natural, bulky nucleotide analogues as substrates.

Within the DNA template itself, certain nucleotide sequence motifs are known to present additional replication challenges to DNA polymerases. Of particular consequence are runs of homopolymers, or short repeated DNA sequences, which can trigger slipped-strand mispairing, or “replication slippage”. Replication slippage is thought to encompass the following steps: (i) copying of the first repeat by the replication machinery, (ii) replication pausing and dissociation of the polymerase from the newly synthesized end, (iii) unpairing of the newly synthesized strand and its pairing with the second repeat, and (iv) resumption of DNA synthesis. Arrest of the replication machinery within a repeated region thus results in misalignment of primer and template. In vivo, misalignment of two DNA strands during replication can lead to DNA rearrangements such as deletions or duplications of varying lengths. In vitro, replication slippage results in replication errors at the site of the slippage event. Such reduction in polymerase processivity, or accuracy, significantly impairs the particular application or desired genetic manipulation.

Thus, new methods and compositions for enhancing polymerase reactions under conditions including one or more reagents with atypical structures are necessary (e.g., in sequencing by expansion (SBX) and other applications in biotechnology and biomedicine, such as DNA amplification, conventional sequencing, labeling, detection, cloning, etc.), and would find value in the art. The present disclosure fulfills these needs and provides further related advantages.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY OF THE DISCLOSURE

The present disclosure provides compounds, compositions and uses thereof that enhance nucleic acid polymerase activity. In certain embodiments polymerase activity is enhanced in polymerization reactions under conditions that introduce one or more challenges to the polymerase, e.g., conditions that include non-natural nucleotide analog substrates or template motifs that impair polymerase processivity. Such enhancement is achieved by supplementing a polymerization reaction with one or more compounds of the present disclosure, which may be referred to herein as “Polymerase Enhancing Molecules” (“PEMs”).

One aspect of the present disclosure is a compound, such as a PEM, having Formula (I):

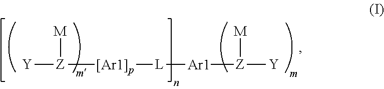

or a solvate, hydrate, tautomer, chelate or salt thereof,
wherein
a is 0 or an integer ranging from 1-4;
a′ is 0 or an integer ranging from 1-4;
m is 1, 2 or 3;
m′ is 1, 2 or 3;
n is 0, 1 or 2;
p is 0, 1 or 2;
Z is a 5-membered or 6-membered heteroaromatic ring including one, two, three, or four heteroatoms selected from O, N, or S;
L is a linking group;
M is, at each occurrence, independently selected from hydrogen, halogen and C₁-C₄ alkyl;
Ar1 is, at each occurrence, independently selected from optionally substituted phenyl, pyridine, bipyridine, tripyridine, pyrazine, pyridazine, furan, dibenzofuran, thiophene, pyrrole, selenophene, naphthalene, fluorene, phenanthrene, cinnoline, phthalazine, quinazoline, quinoxaline, naphthyridine, phenanthroline, purine, and carbazole,
wherein substituents for Ar1 are, at each occurrence, independently selected from halogen, —OH, —CN, —NO₂, C₁-C₆ alkyl, C₁-C₆ haloalkyl, C₁-C₆ heteroalkyl, C₁-C₆ cycloloalkyl, —OR⁰, —CONH₂, —C(O)NR¹R^{1′}, —C(O)(CH₂)ₐNR¹R^{1′}, —NR¹R^{1′}, —NR¹C(O)R³, —C(O)SR³, —COR³, —CO(CH₂)ₐOC(O)R³, —OC(O)R³, —C(O)OR³, —C—O—R³, mercaptan, —R⁴—H, —SOR¹, —S(O)₂R¹, —S(O)₂NR¹R^{1′}, —CH₂—NR¹S(O)₂R³, —NR¹S(O)₂R³, and —C(CH₃)═N-(phenyl)-O—CH₂—C—CH;
R⁰ is, at each occurrence, independently selected from C₁-C₆ alkyl, C₁-C₆ haloalkyl, C₂-C₆ alkenyl, C₂-C₆ alkynyl, substituted or unsubstituted cycloalkyl, substituted or unsubstituted heterocyclyl, substituted or unsubstituted aryl, and substituted or unsubstituted heteroaryl;
R¹ and R^{1′} are, at each occurrence, independently selected from H, hydroxyl, C₁-C₆ alkyl, C₁-C₆ haloalkyl, C₁-C₂₀ heteroalkyl, C₁-C₁₀ heteroalkyl-NH₂, C₂-C₆ alkenyl, C₂-C₆ alkynyl, substituted or unsubstituted cycloalkyl, substituted or unsubstituted heterocyclyl (e.g., substituted with one or more R³ groups), substituted or unsubstituted aryl, substituted or unsubstituted arylalkyl, and substituted or unsubstituted heteroaryl, benzyl, —C(═NH)NH₂, —CH₂CO₂R⁰, —C(H)(CHₐ(COOH))₂, —CH₂C(O)NHCH₂CO₂H, —CH₂CH₂OH, —CH₂CH₂NHC(O)R³, —(CH₂)ₐC(O)NH(CH₂)ₐC(O)OR³, —(CH₂)ₐ—CN, spermine, —(CH₂)ₐ—NH₂, —C(O)R³, —(CH₂)ₐC(O)OR³, —(CH₂)ₐOR³, —C(H)((CH₂)ₐOH)_{a′}, —CHCH(OH)(CH₂)ₐOH, —(CH₂)ₐOH, C₁-C₆—C(O)OH, —(CH₂)ₐ-heterocycle which may be substituted (e.g., such as with an R³ group) or unsubstituted

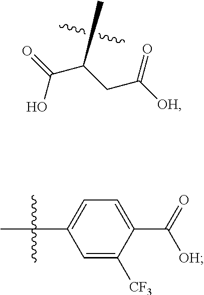

wherein R¹ and R^{1′} together form a substituted or unsubstituted heterocyclic ring, including, but not limited to, azetidine, pyrrolidine, piperidine, piperazine, morpholine,

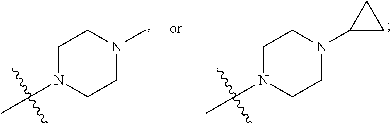

R² is, at each occurrence, independently selected from C₂-C₆ alkyl, C₁-C₆ haloalkyl, C₁-C₆ heteroalkyl, or substituted or unsubstituted cycloalkyl, substituted or unsubstituted heterocyclyl, substituted or unsubstituted aryl, substituted or unsubstituted heteroaryl, and substituted or unsubstituted haloalkoxy;
R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine;
R⁴ is, at each occurrence, independently selected from one or more heteroatom interrupted alkylene, wherein the heteroatom is O, S, NH, or a combination thereof;
Y is, at each occurrence, independently selected from Ar2, —C(O)—Ar2, —(CH₂)ₐAr2, —(CH₂)₃PO(OEt)₂, or —CH₂CO₂Me;
Ar2 is, at each occurrence, independently selected from (i) a substituted or unsubstituted 5- and 6-membered monocyclic aromatic or heteroaromatic ring; (ii) a substituted or unsubstituted 9- and 10-membered fused bicyclic rings comprising two monocyclic rings together, where at least one of the two monocyclic rings is an aromatic or a heteroaromatic ring; and (iii) a substituted or unsubstituted 13- and 14-membered fused tricyclic rings comprising three monocyclic rings together, where at least one of the three monocyclic rings is an aromatic or a heteroaromatic ring;
wherein each Ar2 is independently substituted with G¹, G², G³, G⁴ and G⁵, wherein:
when Ar2 is monosubstituted, G¹ is, at each occurrence, independently selected from oxo, C₁-C₆ alkyl, C₁-C₆ haloalkyl, -E-O—R³, -E-C(R¹)(R^{1′})(R³), —NH₂, —NO₂, —SO₃R³, —SO₃O—, —SO₃N(H)(R¹), -E-C(O)R³, -E-CO₂H, —B(OH)₂, —C(O)NR¹R^{1′}, -E-PO(OR¹)₂, and aryl substituted with G², G³, G⁴ and G⁵;
G², G³, G⁴ and G⁵ are, at each occurrence, independently selected from absent or selected from the groups comprising, halogen, —CN, —NO₂, —SO₃R³, C₁-C₆ alkyl, C₁-C₆ haloalkyl, -E-O—R³, E-(CH₂)ₐC(O)R³, -E-CO₂H, -E-CHO, -E-C(O)R³, -E-C(O)NH(OH), -E-C(O)NHR¹, -E-C(O)N(H)C(H)(R¹)(R^{1′}), -E-C(O)N(R¹)((CH₂)ₐP(O)(O)OH)₂, -E-C(O)N(R¹)((CH₂)aSO₃H), -E-C(O)NR¹R^{1′}, -E-NR¹R^{1′}, -E-OR², —C(O)-heterocycloalkyl (where the heterocycloalkyl is substituted or unsubstituted), —C(O)—N(H)—CH(COOH)((CH₂)ₐ-heteroaryl) (where heteroaryl is substituted or unsubstituted), wherein E is, at each occurrence, independently selected from a direct bond, and C₁-C₆ alkylene.

In some embodiments, the compounds of Formula (I) are in the form of a salt.

In some embodiments, Z is not a triazole. For example, in some embodiments, Z is not a 1,2,3-triazole or a 1,2,4-triazole.

In some embodiments, Z is a diazole (e.g., imidazole, pyrazole, pyrimidine, pyridazine, pyrazine). In other embodiments, Z is pyrrole, thiophene, or triazole (e.g., 1,2,3-triazole; 1,2,4-triazole). In yet other embodiments, Z is thiazole. In yet other embodiments, Z is isoxazole. In even further embodiments, Z is a tetrazole.

In some embodiments, each Z is independently substituted with one “M” moiety. In other embodiments, each Z is independently substituted with two “M” moieties. In yet other embodiments, each Z is independently substituted with three “M” moieties.

In some embodiments, L is a heteroalkylene group of 2 to 10 carbon atoms in length. In other embodiments, L is a heteroalkylene group of 2 to 10 carbon atoms in length, wherein one or more carbon atoms is replaced with at least one heteroatom selected from oxygen, nitrogen, and sulfur.

Non-limiting examples of suitable L groups bridging two Ar1 groups are set forth below:

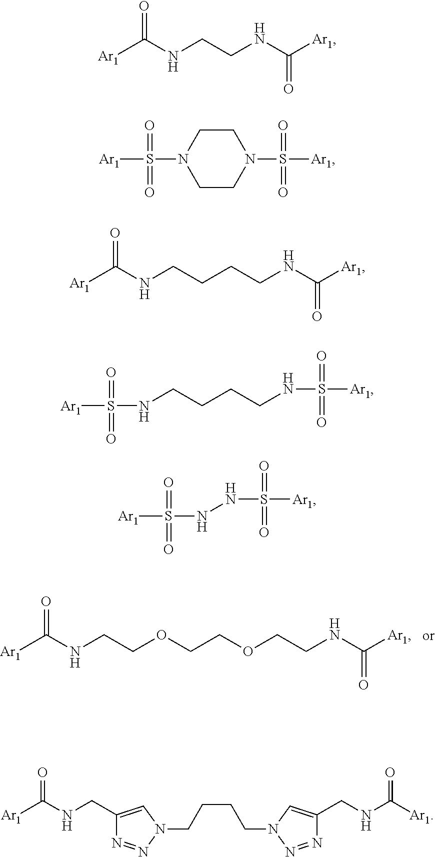

Non-limiting examples of the compounds having Formula (I) are set forth in Table 1, herein.

Another aspect of the present disclosure is a polymer, copolymer, or metallogel of any of the compounds having Formula (I).

Another aspect of the present disclosure is a composition including two or more molecules of any one of the compounds having Formula (I).

Another aspect of the present disclosure is a compound having Formula (IC):

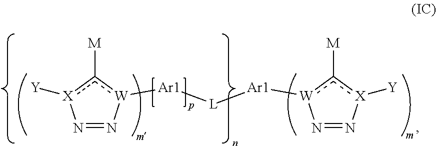

or a solvate, hydrate, tautomer, chelate or salt thereof, wherein:
a is 0 or an integer ranging from 1-4;
a′ is 0 or an integer ranging from 1-4;
m is 1, 2 or 3;
m′ is 1, 2 or 3;
n is 0, 1 or 2;
p is 0, 1 or 2;
W is N when X is C or W is C when X is N;
[symbol] is a single or double bond, wherein the double bond begins at whichever of W or X is carbon;
L is a linking group;
M is, at each occurrence, independently selected from hydrogen, halogen and C₁-C₄ alkyl;
Ar1 is, at each occurrence, independently selected from optionally substituted phenyl, pyridine, bipyridine, tripyridine, pyrazine, pyridazine, furan, dibenzofuran, thiophene, pyrrole, selenophene, naphthalene, fluorene, phenanthrene, cinnoline, phthalazine, quinazoline, quinoxaline, naphthyridine, phenanthroline, purine, and carbazole,
wherein substituents for Ar1 are, at each occurrence, independently selected from halogen, —OH, —CN, —NO₂, C₁-C₆ alkyl, C₁-C₆ haloalkyl, C₁-C₆ heteroalkyl, C₁-C₆ cycloloalkyl, —OR⁰, —CONH₂, —C(O)NR¹R^{1′}, —C(O)(CH₂)ₐNR¹R^{1′}, —NR¹R^{1′}, —NR¹C(O)R³, —C(O)SR³, —COR³, —CO(CH₂)ₐOC(O)R³, —OC(O)R³, —C(O)OR³, —C—O—R³, mercaptan, —R⁴—H, —SOR¹, —S(O)₂R¹, —S(O)₂NR¹R^{1′}, —CH₂—NR¹S(O)₂R³, —NR¹S(O)₂R³, and —C(CH₃)═N-(phenyl)-O—CH₂—C—CH;
R⁰ is, at each occurrence, independently selected from C₁-C₆ alkyl, C₁-C₆ haloalkyl, C₂-C₆ alkenyl, C₂-C₆ alkynyl, substituted or unsubstituted cycloalkyl, substituted or unsubstituted heterocyclyl, substituted or unsubstituted aryl, and substituted or unsubstituted heteroaryl;
R¹ and R^{1′} are, at each occurrence, independently selected from H, hydroxyl, C₁-C₆ alkyl, C₁-C₆ haloalkyl, C₁-C₂₀ heteroalkyl, C₁-C₁₀ heteroalkyl-NH₂, C₂-C₆ alkenyl, C₂-C₆ alkynyl, substituted or unsubstituted cycloalkyl, substituted or unsubstituted heterocyclyl (e.g., substituted with one or more R³ groups), substituted or unsubstituted aryl, substituted or unsubstituted arylalkyl, and substituted or unsubstituted heteroaryl, benzyl, —C(═NH)NH₂, —CH₂CO₂R⁰, —C(H)(CHₐ(COOH))₂, —CH₂C(O)NHCH₂CO₂H, —CH₂CH₂OH, —CH₂CH₂NHC(O)R³, —(CH₂)ₐC(O)NH(CH₂)ₐC(O)OR³, —(CH₂)ₐ—CN, spermine, —(CH₂)ₐ—NH₂, —C(O)R³, —(CH₂)ₐC(O)OR³, —(CH₂)ₐOR³, —C(H)((CH₂)ₐOH)^{a′}, —CHCH(OH)(CH₂)ₐOH, —(CH₂)ₐOH, C₁-C₆—C(O)OH, —(CH₂)ₐ-heterocycle which may be substituted (e.g., such as with an R³ group) or unsubstituted,

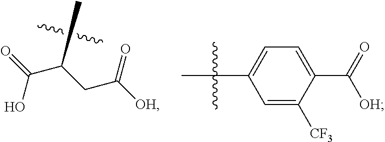

and
wherein R¹ and R^{1′} together form a substituted or unsubstituted heterocyclic ring, including, but not limited to, azetidine, pyrrolidine, piperidine, piperazine, morpholine,

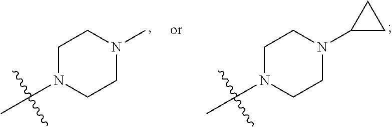

R² is, at each occurrence, independently selected from C₂-C₆ alkyl, C₁-C₆ haloalkyl, C₁-C₆ heteroalkyl, or substituted or unsubstituted cycloalkyl, substituted or unsubstituted heterocyclyl, substituted or unsubstituted aryl, substituted or unsubstituted heteroaryl, and substituted or unsubstituted haloalkoxy;
R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine;
R⁴ is, at each occurrence, independently selected from one or more heteroatom interrupted alkylene wherein the heteroatom is O, S, NH, or a combination thereof;
Y is, at each occurrence, independently selected from Ar2, —C(O)—Ar2, —(CH₂)ₐAr2, —(CH₂)₃PO(OEt)₂, or —CH₂CO₂Me;
Ar2 is, at each occurrence, independently selected from (i) a substituted or unsubstituted 5- and 6-membered monocyclic aromatic or heteroaromatic ring; (ii) a substituted or unsubstituted 9- and 10-membered fused bicyclic rings comprising two monocyclic rings together, where at least one of the two monocyclic rings is an aromatic or a heteroaromatic ring; and (iii) a substituted or unsubstituted 13- and 14-membered fused tricyclic rings comprising three monocyclic rings together, where at least one of the three monocyclic rings is an aromatic or a heteroaromatic ring;
wherein each Ar2 is independently substituted with G¹, G², G³, G⁴ and G⁵, wherein:
when Ar2 is monosubstituted, G¹ is, at each occurrence, independently selected from oxo, C₁-C₆ alkyl, C₁-C₆ haloalkyl, -E-O—R³, -E-C(R¹)(R^{1′})(R³), —NH₂, —NO₂, —SO₃R³, —SO₃O⁻, —SO₃N(H)(R¹), -E-C(O)R³, -E-CO₂H, —B(OH)₂, —C(O)NR¹R^{1′}, -E-PO(OR¹)₂, and aryl substituted with G², G³, G⁴ and G⁵;
G², G³, G⁴ and G⁵ are, at each occurrence, independently selected from absent or selected from the groups comprising, halogen, —CN, —NO₂, —SO₃R³, C₁-C₆ alkyl, C₁-C₆ haloalkyl, -E-O—R³, E-(CH₂)ₐC(O)R³, -E-CO₂H, -E-CHO, -E-C(O)R³, -E-C(O)NH(OH), -E-C(O)NHR¹, -E-C(O)N(H)C(H)(R¹)(R^{1′}), -E-C(O)N(R¹)((CH₂)ₐP(O)(O)OH)₂, -E-C(O)N(R¹)((CH₂)ₐSO₃H), -E-C(O)NR¹R^{1′}, -E-NR¹R^{1′}, -E-OR², —C(O)-heterocycloalkyl (where the heterocycloalkyl is substituted or unsubstituted), —C(O)—N(H)—CH(COOH)((CH₂)ₐ-heteroaryl) (where heteroaryl is substituted or unsubstituted), wherein E is, at each occurrence, independently selected from a direct bond, and C₁-C₆ alkylene.

Another aspect of the present disclosure is a polymer, copolymer, or metallogel of any of the compounds having Formula (IC).

Another aspect of the present disclosure is a composition comprising two or more molecules of any one of the compounds having Formula (IC).

Another aspect of the present disclosure is a method of enhancing a nucleic acid polymerase reaction, the method including the steps of forming a nucleic acid polymerase reaction composition including a template nucleic acid, a nucleic acid polymerase, a mixture of nucleotides and/or nucleotide analogs, at least one PEM (such as those compounds having any one of Formulas (I), (IA), (IB), (IC), and/or (ID) as set forth herein); and incubating the nucleic acid polymerase reaction composition under conditions allowing a nucleic acid polymerization reaction. It is believed that the at least one PEM increases the processivity, rate, and/or fidelity of the nucleic acid polymerase reaction. In some embodiments, the at least one PEM increases the length of a resulting nucleic acid product compared to a nucleic acid polymerase reaction lacking the at least one PEM.

In some embodiments, the nucleic acid polymerase is a DNA polymerase. In some embodiments, the DNA polymerase is DPO₄ or a variant thereof. In other embodiments, the mixture of nucleotides or nucleotide analogs is a mixture of nucleotide analogs comprising nucleoside triphosphoramidates, wherein each of the nucleoside triphosphoramidates includes a nucleobase selected from the group consisting of adenine, guanine, thymine, and cytosine and a polymeric tether moiety, wherein a first end of the polymeric tether moiety is attached to the nucleobase and a second end of the polymeric tether moiety is attached to the alpha phosphate of the nucleoside triphosphoramidate to provide for expansion of the nucleotide analogs by cleavage of the phosphoramidate bond.

In some embodiments, the nucleic acid polymerization reaction produces an expandable polymer of nucleotide analogs, wherein the expandable polymer encodes the nucleobase sequence information of the template nucleic acid. In other embodiments, the conditions for allowing a nucleic acid polymerization reaction includes a suitable polymerization buffer and an oligonucleotide primer. In further embodiments, the suitable buffer includes one or more components selected from MnCl₂, a buffer, a salt, a sugar, a single-strand binding protein (SSB), imidazole, pyrazole, triazole, betaine, a molecular crowding agent, dimethyl sulfoxide (DMSO), an alkanediol, glycerol, N-methyl-2-pyrrolidone (NMP), acetamide, butylated hydroxyanisole (BHA), a polyphosphate, and/or urea. In other embodiments, the reaction mixture further includes a nucleic acid intercalating agent. In other embodiments, the reaction mixture further includes a polyanion recognition moiety. In further embodiments, the mixture of nucleotides or nucleotide analogs includes nucleotide analogs comprising a detectable label. In yet other embodiments, the detectable label is an optically detectable label selected from the group consisting of luminescent, chemiluminescent, fluorescent, fluorogenic, chromophoric or chromogenic labels.

Another aspect of the present disclosure is the use of a PEM (including any of the compounds disclosed herein having any one of Formulas (I), (IA), (IB), (IC), and/or (ID) set forth herein) in enhancing a nucleic acid polymerase reaction.

Another aspect of the present disclosure is a composition including at least one PEM (including any of the compounds disclosed herein having any one of Formulas (I), (IA), (IB), (IC), and/or (ID) set forth herein), and a mixture of nucleotide analogs. It is believed that this composition is useful, e.g., when combined with a polymerase, wherein the at least one PEM increases the number and accuracy of nucleotide analogs incorporated into a daughter strand during a template-dependent polymerization reaction relative to an identical polymerization reaction absent the present of the at least one PEM. In other embodiments, the at least one PEM comprises a plurality of PEMs.

Optionally, the mixture of nucleotide analogs comprises nucleoside triphosphoramidates, wherein each of the nucleoside triphosphoramidates comprises a nucleobase selected from the group consisting of adenine, guanine, thymine, and cytosine and a polymeric tether moiety, wherein a first end of the polymeric tether moiety is attached to the nucleobase and a second end of the polymeric ether moiety is attached to the alpha phosphate of the nucleoside triphosphoramidate to provide for expansion of the nucleotide analogs by cleavage of the phosphoramidate bond. In other embodiments, the composition further includes a buffer which includes one or more components selected from MnCl₂, a buffer, a salt, a sugar, a single-strand binding protein (SSB), imidazole, pyrazole, triazole, betaine, a molecular crowding agent, dimethyl sulfoxide (DMSO), an alkanediol, glycerol, N-methyl-2-pyrrolidone (NMP), acetamide, butylated hydroxyanisole (BHA), a polyphosphate, and/or urea. In other embodiments, the composition further includes a single-strand binding protein (SSB). In other embodiments, the composition further includes urea. In some embodiments, the mixture of nucleotide analogs includes nucleotide analogs including a detectable label. In some embodiments, the detectable label is an optically detectable label selected from the group consisting of luminescent, chemiluminescent, fluorescent, fluorogenic, chromophoric or chromogenic labels.

Another aspect of the present disclosure is a method of sequencing a DNA or RNA template, the method including the steps of forming a DNA polymerase reaction composition including the DNA or RNA template, a replication primer that complexes with the template, a DNA polymerase, a mixture of nucleotides or nucleotide analogs, and at least one PEM (including any of the compounds disclosed herein having any one of Formulas (I), (IA), (IB), (IC), and/or (ID) as set forth herein), and incubating the DNA polymerase reaction composition under conditions allowing a DNA polymerization reaction, wherein the at least one PEM increases the rate, fidelity or processivity of the DNA polymerase reaction. In some embodiments, the method may further include determining the sequence of the nucleotides or nucleotide analogs in the resulting polymer of nucleotides or nucleotide analogs. In some embodiments, the at least one PEM is a compound having any one of Formulas (I), (IA), (IB), (IC), and (ID). In other embodiments, the at least one PEM is any one of the compounds set forth herein in Table 1. In other embodiments, the mixture of nucleotide analogs comprises nucleoside triphosphoramidates, wherein each of the nucleoside triphosphoramidates comprises a nucleobase selected from the group consisting of adenine, guanine, thymine, and cytosine and a polymeric tether moiety, wherein a first end of the polymeric tether moiety is attached to the nucleobase and a second end of the polymeric ether moiety is attached to the alpha phosphate of the nucleoside triphosphoramidate to provide for expansion of the nucleotide analogs by cleavage of the phosphoramidate bond. In other embodiments, the DNA polymerase is DP04 or a variant thereof. In other embodiments, the resulting polymer of nucleotide analogs is an expandable polymer. In other embodiments, the method further includes the step of contacting the expandable polymer with a phosphoramidate cleavage agent to produce an expanded polymer of nucleotide analogs. In certain embodiments, the polymeric tether moiety of each of the nucleotide analogs comprises a reporter moiety unique to the nucleobase of the analog. In other embodiments, the reporter moieties produce a characteristic electronic signal. In yet other embodiments, the step of determining the sequence of the nucleotide analogs includes the step of translocating the expanded polymer of nucleotide analogs through a nanopore.

Another aspect of the present disclosure is a use of a PEM (as those compounds having any one of Formulas (I), (IA), (IB), (IC), and/or (ID) as set forth herein) in sequencing a DNA or RNA template

Another aspect of the present disclosure is a composition comprising a PEM (as those compounds having any one of Formulas (I), (IA), (IB), (IC), and/or (ID) as set forth herein) and a polynucleotide.

Another aspect of the present disclosure is a composition comprising a PEM (as those compounds having any one of Formulas (I), (IA), (IB), (IC), and/or (ID) as set forth herein) a polypeptide, e.g., a polypeptide such as an enzyme, where the enzyme may be a nucleic acid polymerase.

The following are exemplary, non-limiting numbered embodiments of the present disclosure. Also, unless otherwise specifically mentioned, each atom identified in a chemical formula may be any of the isotopes of that atom. For example, the designation C (carbon) includes ¹²C, ¹³C, or ¹⁴C and mixtures thereof, particularly natural abundance isotope mixtures, while H (hydrogen) includes ¹H, ²H and ³H and mixtures thereof, and O (oxygen) includes ¹⁶O and ¹⁸O and mixtures thereof, and N (nitrogen) includes ¹⁴N and ¹⁵N and mixtures thereof, etc. for other atoms.
Embodiment 1) A compound having Formula (I).

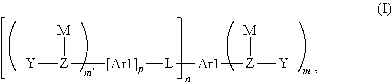

or a solvate, hydrate, tautomer, chelate or salt thereof,
wherein
a is 0 or an integer ranging from 1-4;
a′ is 0 or an integer ranging from 1-4;
m is 1, 2 or 3;
m′ is 1, 2 or 3;
n is 0, 1 or 2;
p is 0, 1 or 2;
Z is a 5-membered or 6-membered heteroaromatic ring including one, two, three, or four heteroatoms selected from O, N, or S, where each Z may be independently substituted with one or more halogens and/or one or more C₁-C₄ alkyl groups;
L is a linking group;
M is, at each occurrence, independently selected from hydrogen, halogen and C₁-C₄ alkyl;
Ar1 is, at each occurrence, independently selected from optionally substituted phenyl, pyridine, bipyridine, tripyridine, pyrazine, pyridazine, furan, dibenzofuran, thiophene, pyrrole, selenophene, naphthalene, fluorene, phenanthrene, cinnoline, phthalazine, quinazoline, quinoxaline, naphthyridine, phenanthroline, purine, and carbazole,
wherein substituents for Ar1 are, at each occurrence, independently selected from halogen, —OH, —CN, —NO₂, C₁-C₆ alkyl, C₁-C₆ haloalkyl, C₁-C₆ heteroalkyl, C₁-C₆ cycloloalkyl, —OR⁰, —CONH₂, —C(O)NR¹R^{1′}, —C(O)(CH₂)ₐNR¹R^{1′}, —NR¹R^{1′}, —NR¹C(O)R³, —C(O)SR³, —COR³, —CO(CH₂)ₐOC(O)R³, —OC(O)R³, —C(O)OR³, —C—O—R³, mercaptan, —R⁴—H, —SOR¹, —S(O)₂R¹, —S(O)₂NR¹R^{1′}, —CH₂—NR¹S(O)₂R³, —NR¹S(O)₂R³, and —C(CH₃)═N-(phenyl)-O—CH₂—C—CH;
R⁰ is, at each occurrence, independently selected from C₁-C₆ alkyl, C₁-C₆ haloalkyl, C₂-C₆ alkenyl, C₂-C₆ alkynyl, substituted or unsubstituted cycloalkyl, substituted or unsubstituted heterocyclyl, substituted or unsubstituted aryl, and substituted or unsubstituted heteroaryl;
R¹ and R^{1′} are, at each occurrence, independently selected from H, hydroxyl, C₁-C₆ alkyl, C₁-C₆ haloalkyl, C₁-C₂₀ heteroalkyl, C₁-C₁₀ heteroalkyl-NH₂, C₂-C₆ alkenyl, C₂-C₆ alkynyl, substituted or unsubstituted cycloalkyl, substituted or unsubstituted heterocyclyl (e.g., substituted with one or more R³ groups), substituted or unsubstituted aryl, substituted or unsubstituted arylalkyl, and substituted or unsubstituted heteroaryl, benzyl, —C(═NH)NH₂, —CH₂CO₂R⁰, —C(H)(CHₐ(COOH))₂, —CH₂C(O)NHCH₂CO₂H, —CH₂CH₂OH, —CH₂CH₂NHC(O)R³, —(CH₂)ₐC(O)NH(CH₂)ₐC(O)OR³, —(CH₂)ₐ—CN, spermine, —(CH₂)ₐ—NH₂, —C(O)R³, —(CH₂)ₐC(O)OR³, —(CH₂)ₐOR³, —C(H)((CH₂)ₐOH)_{a′}, —CHCH(OH)(CH₂)ₐOH, —(CH₂)ₐOH, C₁-C₆—C(O)OH, —(CH₂)ₐ-heterocycle which may be substituted (e.g., such as with an R³ group) or unsubstituted,

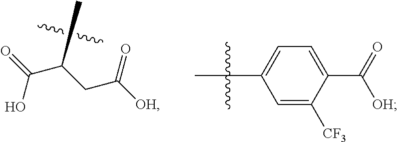

and
wherein R¹ and R^{1′} together form a substituted or unsubstituted heterocyclic ring, including, but not limited to, azetidine, pyrrolidine, piperidine, piperazine, morpholine,

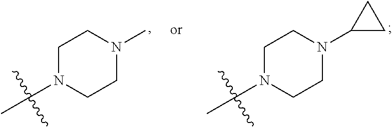

R² is, at each occurrence, independently selected from C₂-C₆ alkyl, C₁-C₆ haloalkyl, C₁-C₆ heteroalkyl, or substituted or unsubstituted cycloalkyl, substituted or unsubstituted heterocyclyl, substituted or unsubstituted aryl, substituted or unsubstituted heteroaryl, and substituted or unsubstituted haloalkoxy;
R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine;
R⁴ is, at each occurrence, independently selected from one or more heteroatom interrupted alkylene wherein the heteroatom is O, S, NH, or a combination thereof;
Y is, at each occurrence, independently selected from Ar2, —C(O)—Ar2, —(CH₂)ₐAr2, —(CH₂)₃PO(OEt)₂, or —CH₂CO₂Me;
Ar2 is, at each occurrence, independently selected from (i) a substituted or unsubstituted 5- and 6-membered monocyclic aromatic or heteroaromatic ring; (ii) a substituted or unsubstituted 9- and 10-membered fused bicyclic rings comprising two monocyclic rings together, where at least one of the two monocyclic rings is an aromatic or a heteroaromatic ring; and (iii) a substituted or unsubstituted 13- and 14-membered fused tricyclic rings comprising three monocyclic rings together, where at least one of the three monocyclic rings is an aromatic or a heteroaromatic ring;
wherein each Ar2 is independently substituted with G¹, G², G³, G⁴ and G⁵, wherein:
when Ar2 is monosubstituted, G¹ is, at each occurrence, independently selected from oxo, C₁-C₆ alkyl, C₁-C₆ haloalkyl, -E-O—R³, -E-C(R¹)(R^{1′})(R³), —NH₂, —NO₂, —SO₃R³, —SO₃O⁻, —SO₃N(H)(R¹), -E-C(O)R³, -E-CO₂H, —B(OH)₂, —C(O)NR¹R^{1′}, -E-PO(OR¹)₂, and aryl substituted with G², G³, G⁴ and G⁵;
G², G³, G⁴ and G⁵ are, at each occurrence, independently selected from absent or selected from the groups comprising, halogen, —CN, —NO₂, —SO₃R³, C₁-C₆ alkyl, C₁-C₆ haloalkyl, -E-O—R³, E-(CH₂)ₐC(O)R³, -E-CO₂H, -E-CHO, -E-C(O)R³, -E-C(O)NH(OH), -E-C(O)NHR¹, -E-C(O)N(H)C(H)(R¹)(R^{1′}), -E-C(O)N(R¹)((CH₂)ₐP(O)(O)OH)₂, -E-C(O)N(R¹)((CH₂)ₐSO₃H), -E-C(O)NR¹R^{1′}, -E-NR¹R^{1′}, -E-OR², —C(O)-heterocycloalkyl (where the heterocycloalkyl is substituted or unsubstituted), —C(O)—N(H)—CH(COOH)((CH₂)ₐ-heteroaryl) (where heteroaryl is substituted or unsubstituted), wherein E is, at each occurrence, independently selected from a direct bond, and C₁-C₆ alkylene.
Embodiment 2) The compound of embodiment 1, wherein the compound of Formula (I) has any one of Formulas (IA) or (IB):

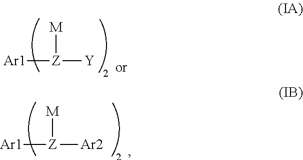

or a salt, solvate, hydrate, tautomer, or chelate thereof,
wherein Ar1, Ar2, Z, and Y are as defined above.
Embodiment 3The compound of embodiment 1, wherein the compound of Formula (I) has Formula (IC):

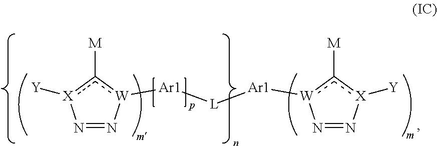

or a solvate, hydrate, tautomer, chelate or salt thereof,
wherein
W is N when X is C, or W is C when X is N;
[symbol] is a single or double bond, wherein the double bond begins at whichever of
W or X is carbon; and
m, m′, n, p, L, and Y are as defined above.
Embodiment 4) The compound of any one of embodiments 1-3, wherein Ar1 is monocyclic heterocyclic aryl.
Embodiment 5) The compound of any one of embodiments 1-3, wherein Ar1 is selected from:

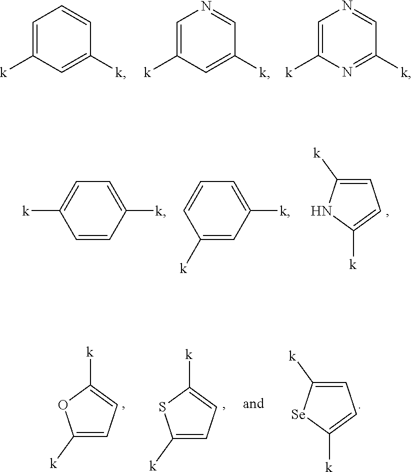

wherein each group “Z” of Formulas (I), (IA), or (IB) is located at positions “k” on Ar1; or wherein each triazole of Formula (IC) is located at positions “k” on Ar1.
Embodiment 6) The compound of any one of embodiments 1-3, wherein Ar1 is bicyclic aryl.
Embodiment 7) The compound of any one of embodiments 1-3, wherein Ar1 is a bicyclic carbocyclic aryl selected from:

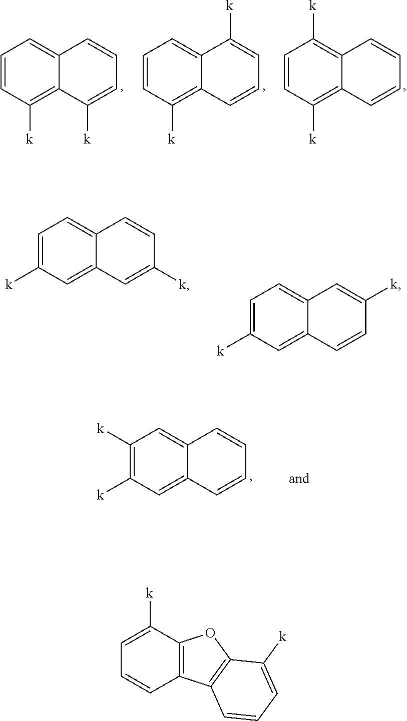

wherein each group “Z” of Formulas (I), (IA), or (IB) is located at positions “k” on Ar1; or wherein each triazole of Formula (IC) is located at positions “k” on Ar1.
Embodiment 8) The compound of any one of embodiments 1-3, wherein Ar1 is a bicyclic heterocyclic aryl selected from:

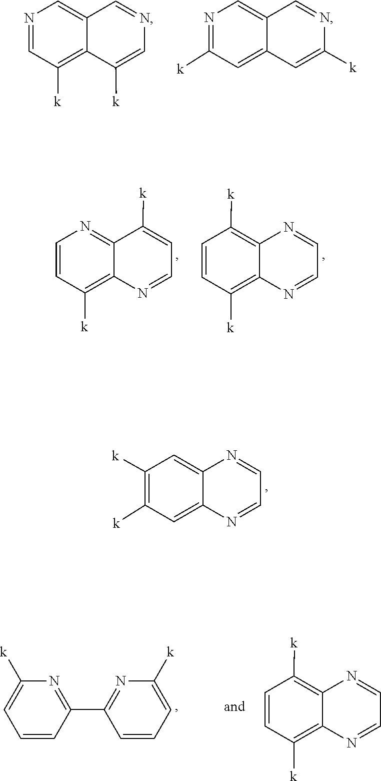

wherein each group “Z” of Formulas (I), (IA), or (IB) is located at positions “k” on Ar1; or wherein each triazole of Formula (IC) is located at positions “k” on Ar1.
Embodiment 9) The compound of any one of embodiments 1-3, wherein Ar1 is tricyclic aryl.
Embodiment 10) The compound of embodiment 9, wherein the tricyclic aryl is selected from:

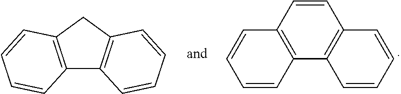

Embodiment 11) The compound of embodiment 9, wherein the tricyclic aryl selected from:

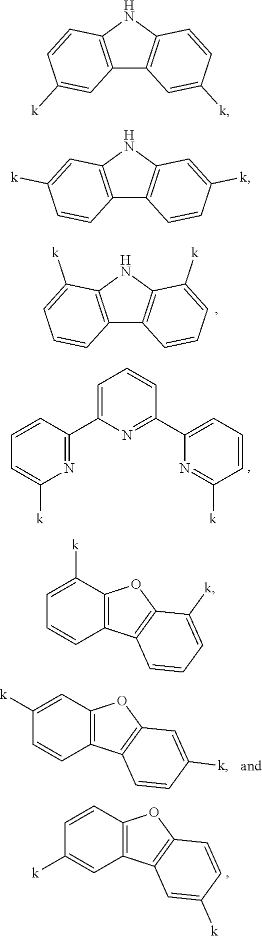

wherein each group “Z” of Formulas (I), (IA), or (IB) is located at positions “k” on Ar1; or wherein each triazole of Formula (IC) is located at positions “k” on Ar1.
Embodiment 12) The compound of embodiment 1, wherein Ar1 is a tricyclic heteroaryl selected from

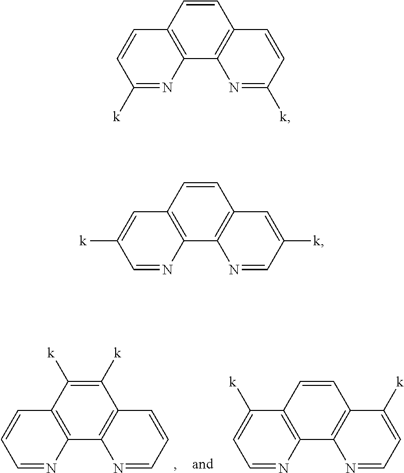

wherein each group “Z” of Formulas (I), (IA), or (IB) is located at positions “k” on Ar1; or wherein each triazole of Formula (IC) is located at positions “k” on Ar1.
Embodiment 13) The compound of any one of the preceding embodiments, wherein Ar2 is a substituted or unsubstituted 5-membered monocyclic aromatic ring selected from the group consisting of thiophene, 1,2-thiazole, 1,3-thiazole, furan, 1,2-oxazole, 1,3-oxazole, 1H-pyrrole, 1H-pyrazole, oxadiazole, thiadiazole, 1,2,4-triazole, 1,2,3-triazole and 1H-imidazole.
Embodiment 14) The compound of any one of embodiments 1-12, wherein Ar2 is a substituted or unsubstituted 6-membered monocyclic aromatic ring selected from the group consisting of benzene, pyridine, pyridazine, pyrimidine and pyrazine.
Embodiment 15) The compound of any one of embodiments 1-12, wherein Ar2 is a substituted or unsubstituted 9-membered fused bicyclic aromatic ring system selected from the group consisting of benzofuran, 1,3-benzoxazole, furo[3,2-b]pyridine, furo[3,2-c]pyridine, furo[2,3-c]pyridine, furo[2,3-b]pyridine, indole, 1H-benzimidazole, 1H-pyrrolo[3,2-b]pyridine, 1H-pyrrolo[3,2-c]pyridine, 1H-pyrrolo[2,3-c]pyridine, 1H-pyrrolo[2,3-b]pyridine, benzothiophene, 1,3-benzothiazole, thienol[3,2-b]pyridine, thieno[3,2-c]pyridine, thieno[2,3-c]pyridine, benzoxadiazole, benzothiadiazole, benzisoxazole, benzotriazole and thieno[2,3-b]pyridine.
Embodiment 16) The compound of any one of embodiments 1-12, wherein Ar2 is a substituted or unsubstituted 10-membered fused bicyclic aromatic ring system selected from the group consisting of naphthylene, quinoline, quinazoline, quinoxaline, 1,5-naphthyridine, 1,6-naphthyridine, 1,7-naphthyridine, 1,8-naphthyridine, isoquinoline, phthalazine, 2,6-naphthyridine and 2,7-naphthyridine.
Embodiment 17) The compound of any one of embodiments 1-12, wherein Ar2 is a substituted or unsubstituted pyridinyl ring selected from

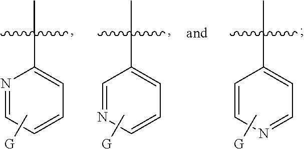

wherein the substituent G (where G may be any one of G¹, G², G³, G⁴ and G⁵ as defined above) is present 0, 1, or 2 times on the pyridinyl ring.
Embodiment 18) The compound of any one of embodiments 1-12, wherein Ar2 is a phenyl ring having the formula

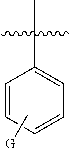

wherein the substituent G (or G¹, G², G³, G⁴ and G⁵ as defined herein) is present 0, 1 or 2 times on the phenyl ring. In one embodiment, G is aryl (such as phenyl) substituted with G², G³, G⁴ and G⁵.
Embodiment 19) The compound of any one of embodiments 1-12, wherein Ar2 is a phenyl ring selected from

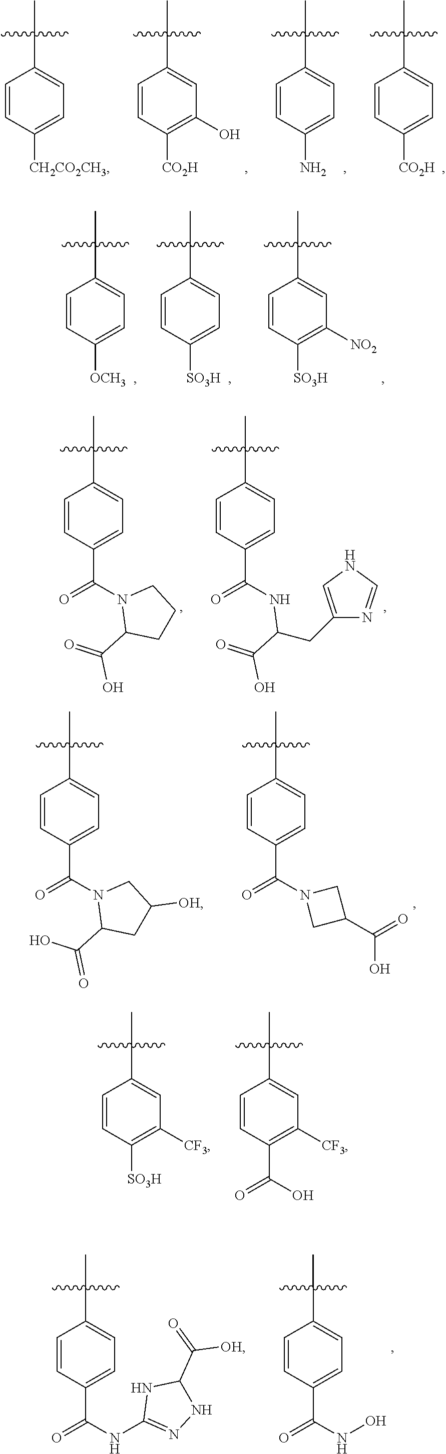

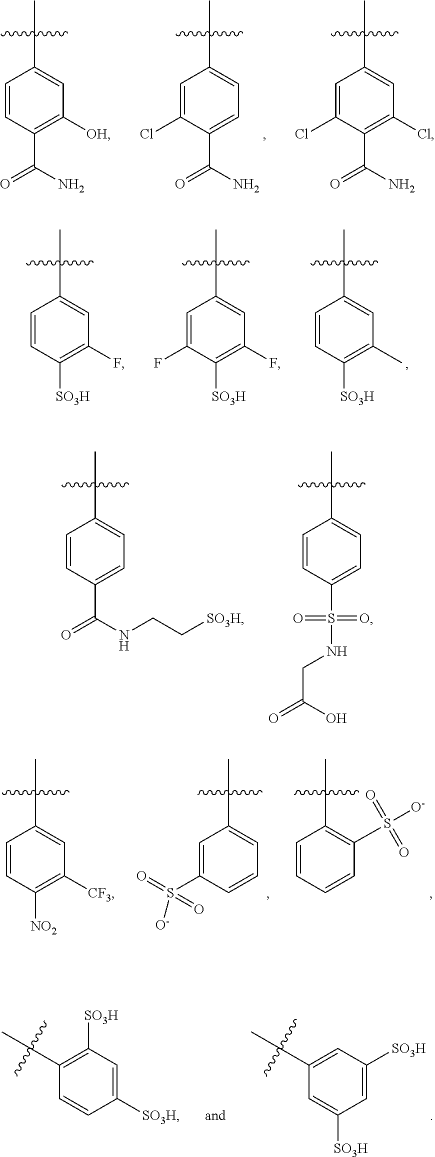

Embodiment 20) The compound of embodiments 1-18, wherein Ar2 includes one or more amino substituents.
Embodiment 21) The compound of embodiments 1-18, wherein Ar2 includes one or more methoxy substituents.
Embodiment 22) The compound of embodiments 1-18, wherein Ar2 includes one or more carboxylic acid substituents.
Embodiment 23) The compound of embodiments 1-18, wherein Ar2 includes one or more —CH₂—CO₂—CH₃ substituents.
Embodiment 24) The compound of embodiments 1-18, wherein Ar2 includes one or more trifluoromethyl substituents.
Embodiment 25) The compound of embodiments 1-18, wherein Ar2 includes one or more hydroxyl substituents.
Embodiment 26) The compound of embodiments 1-18, wherein Ar2 includes at least two substituents, where a first substituent is carboxylic acid, and second substituent is hydroxyl.
Embodiment 27) The compound of embodiments 1-18, wherein Ar2 includes at least two substituents, where a first substituent is carboxylic acid, and a second substituent is trifluoromethyl.
Embodiment 28) The compound of embodiments 1-18, wherein Ar2 includes at least one sulfonic acid group (—SO₃H) or a salt thereof (—SO₂O⁻).
Embodiment 29) The compound of embodiments 1-19, wherein Ar2 includes two substituents, wherein one of the two substituents is a sulfonic acid group (—SO₃H) or a salt thereof (—SO₂O⁻).
Embodiment 30) The compound of any one of the preceding embodiments where the compound is in the form of a chelate.
Embodiment 31) The compound of embodiment 30, wherein the chelate is a copper chelate.
Embodiment 32) The compound of any one of the preceding embodiments where the compound is in the form of a salt.
Embodiment 33) The compound of any one of the preceding embodiments, wherein the compound has a logP of at least 4.9.
Embodiment 34) The compound of embodiment 1, wherein the compound has one of the formulas:

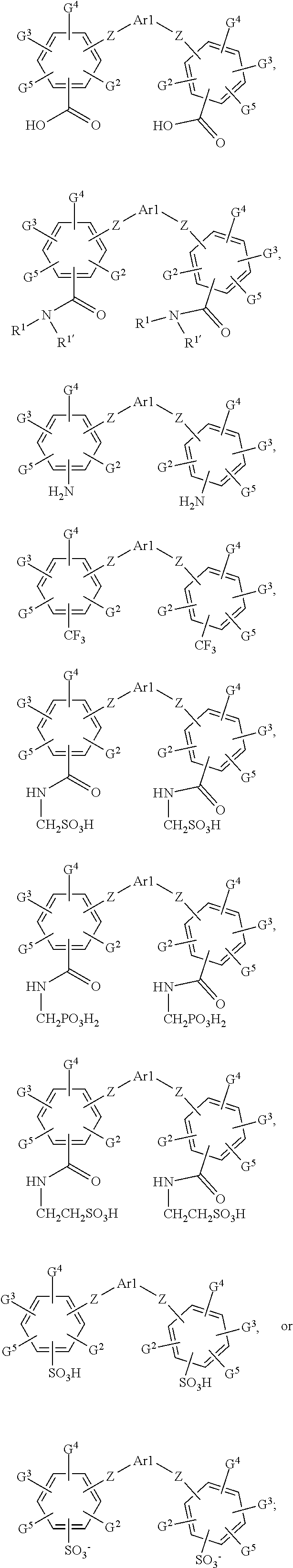

or a salt or hydrate thereof.
Embodiment 35) The compound of embodiment 1, wherein the compound has one of the

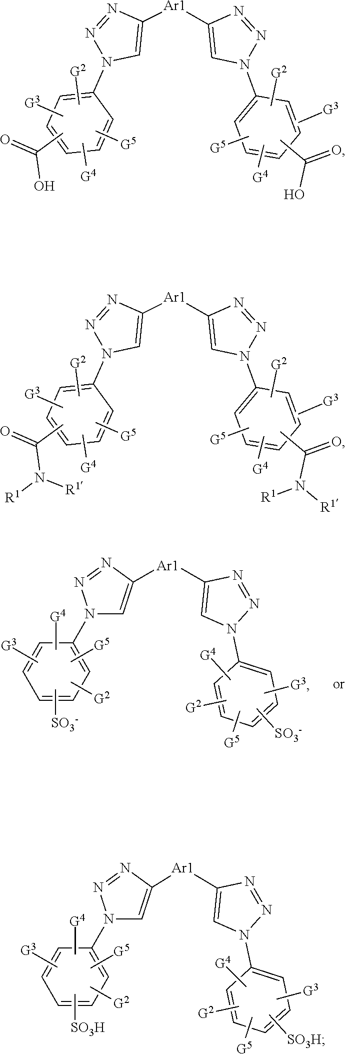

or a salt or hydrate thereof
Embodiment 36) The compound of embodiment 1, wherein Ar2 includes at least two substituents selected from hydroxyl, carboxylic acid carboxamide, and trifluoromethyl.
Embodiment 37) The compound of embodiment 1, wherein the compound has one of the

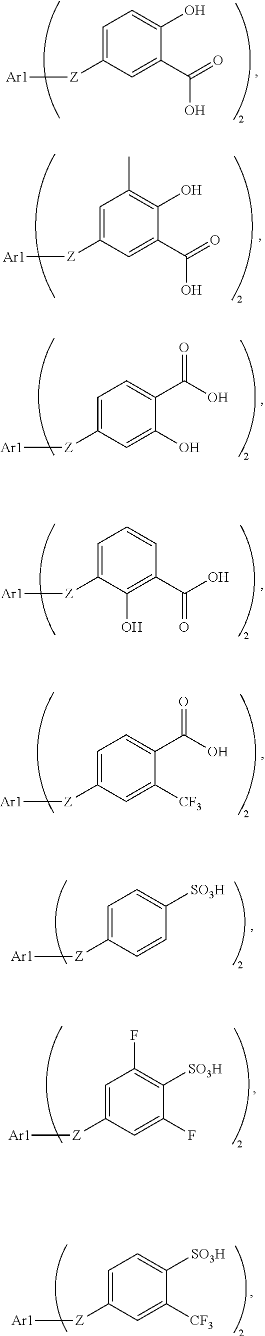

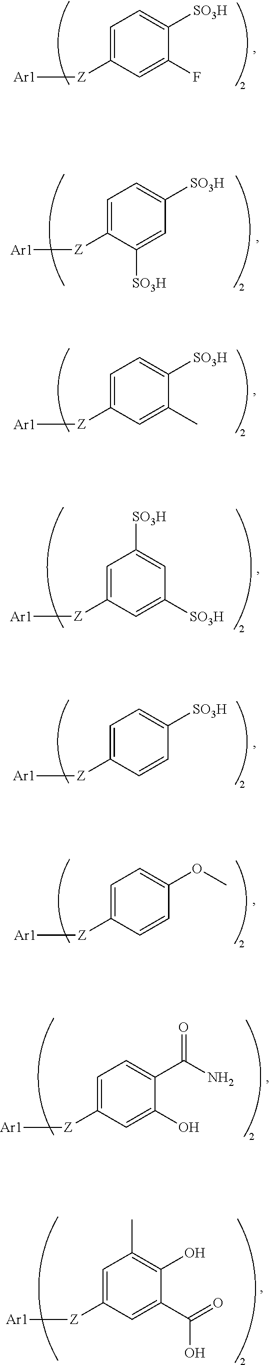

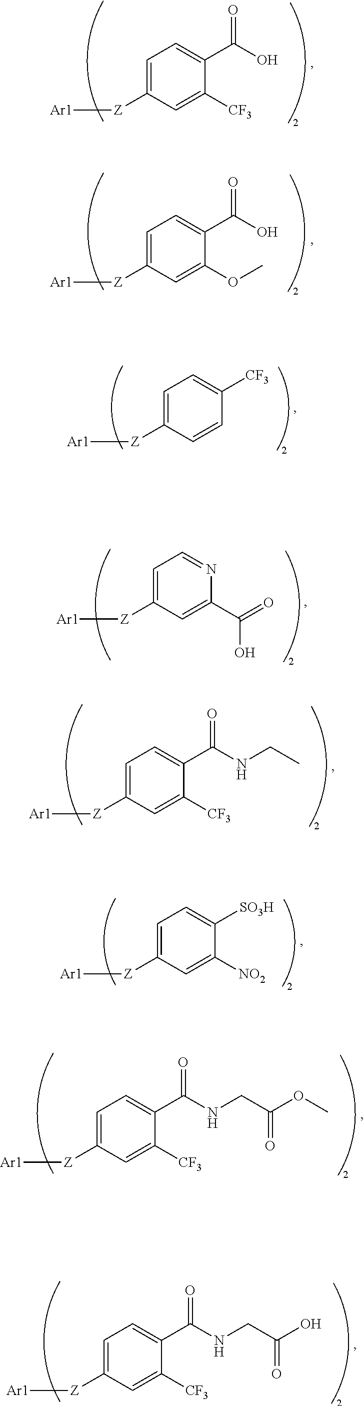

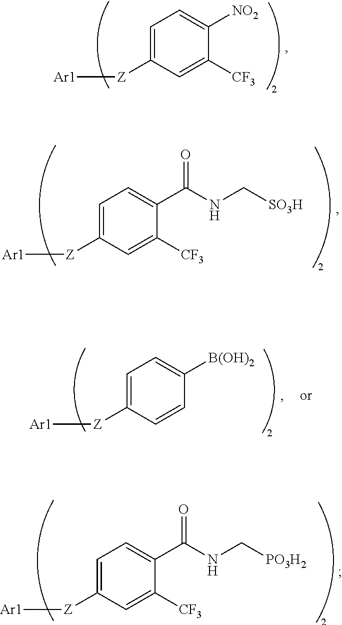

or a salt or hydrate thereof.
Embodiment 38) The compound of embodiment 1, wherein the compound has one of the formulas.

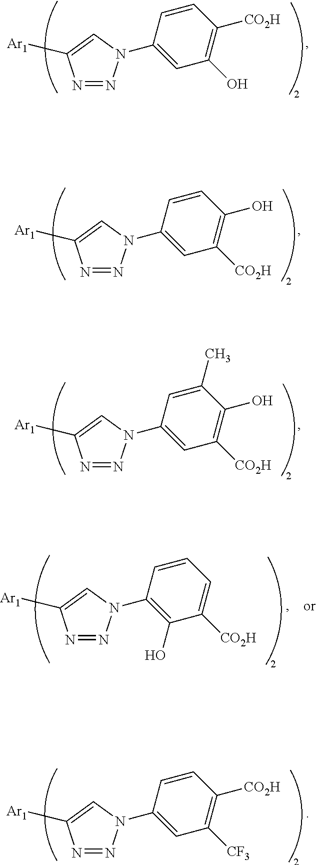

Embodiment 39) The compound of embodiment 1, wherein the compound has one of the formulas:

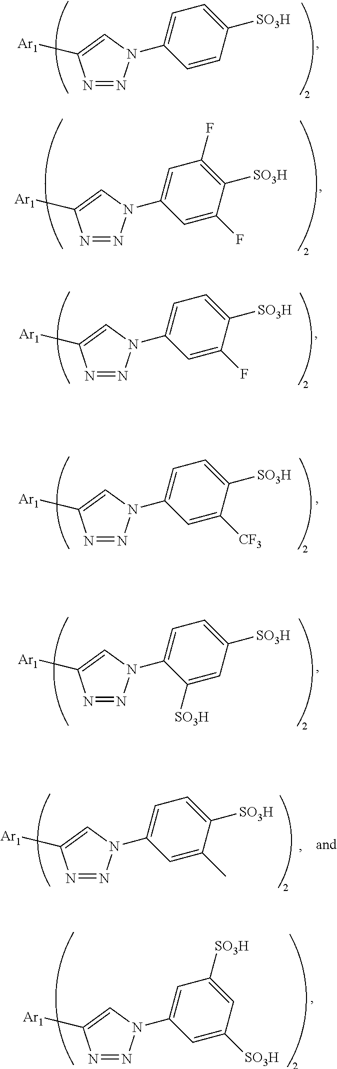

or a salt or hydrate thereof.
Embodiment 40) The compound of embodiment 1, wherein the compound has one of the formulas:

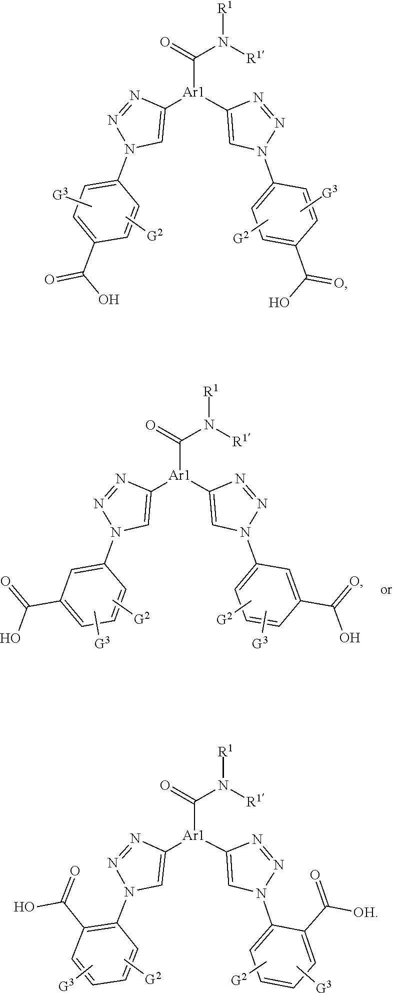

Embodiment 41) The compound of embodiment 1, wherein the compound has one of the formulas.

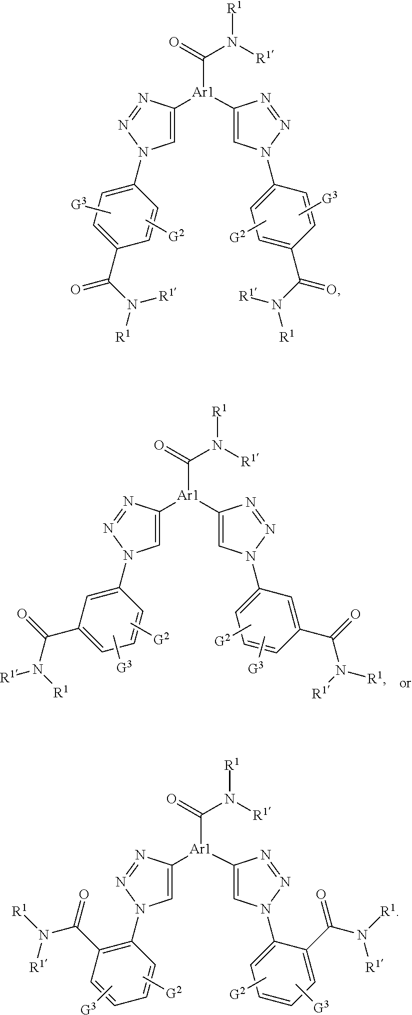

Embodiment 42) The compound of embodiment 1, wherein the compound has one of the formulas:

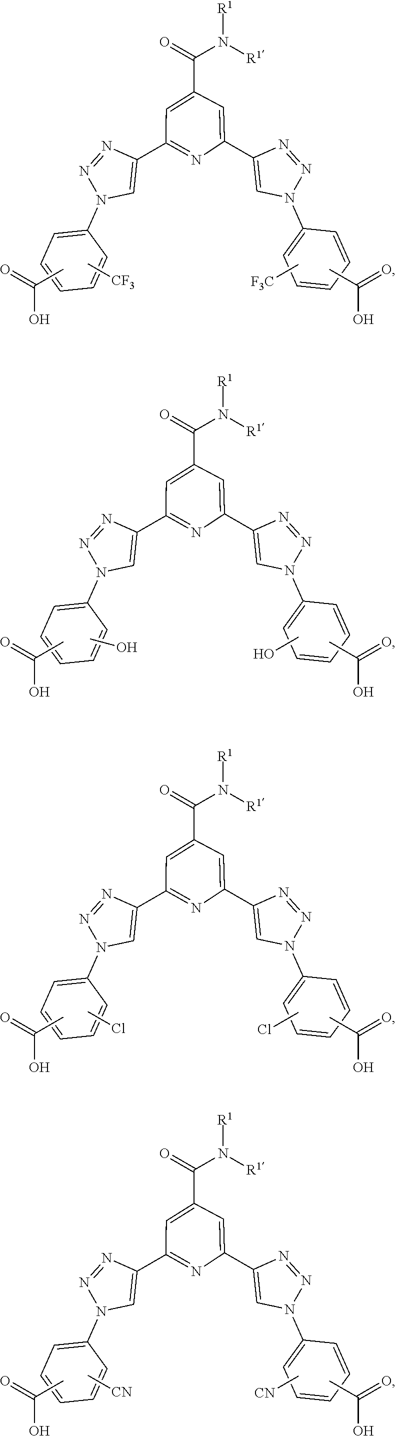

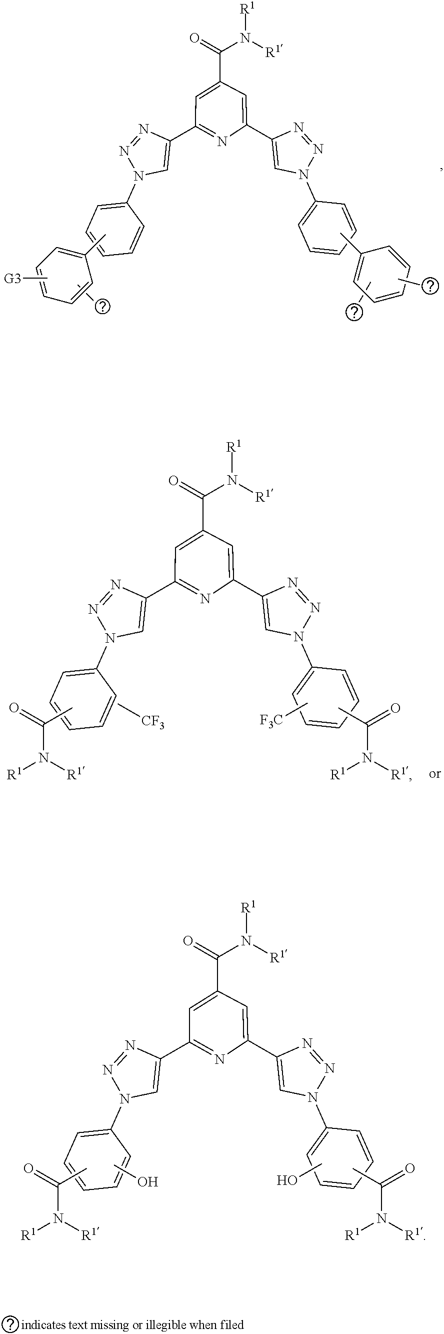

Embodiment 43) The compound of embodiment 1, wherein the compound has one of the formulas:

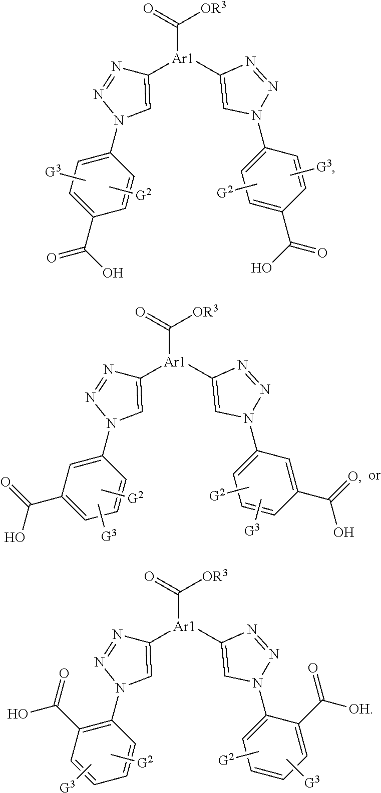

Embodiment 44) The compound of embodiment 1, wherein the compound has one of the formulas:

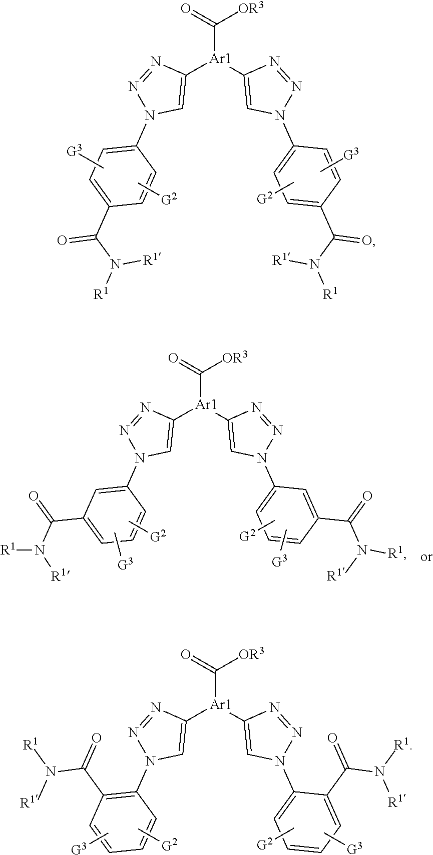

Embodiment 45) The compound of embodiment 1, wherein the compound has one of the formulas:

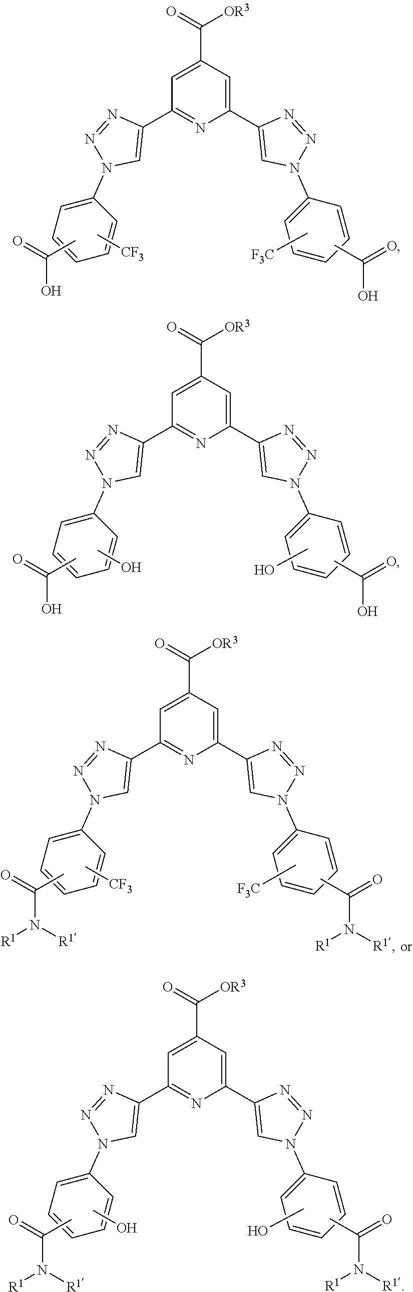

Embodiment 46) The compound of embodiment 1, wherein the compound has one of the formulas:

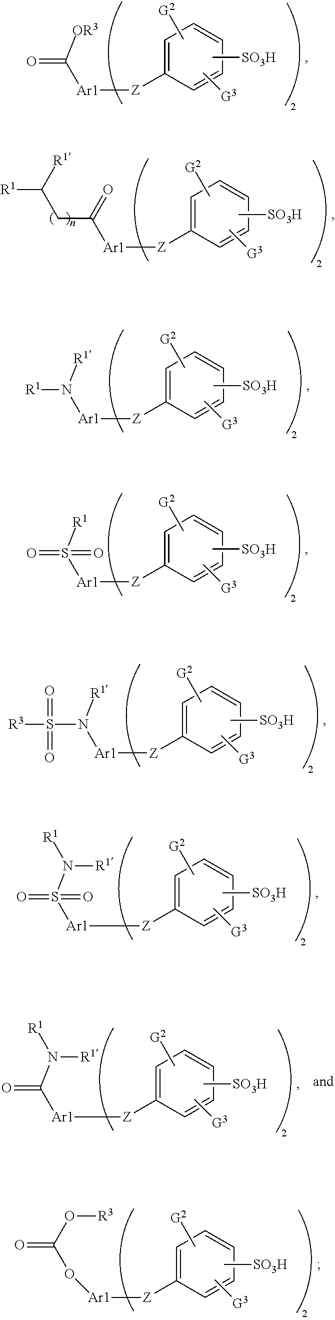

or a salt or hydrate thereof.
Embodiment 47) The compound of embodiment 1, wherein the compound has one of the formulas:

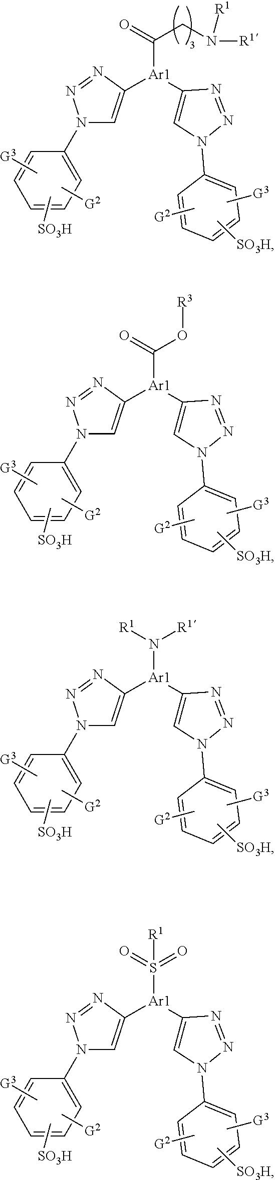

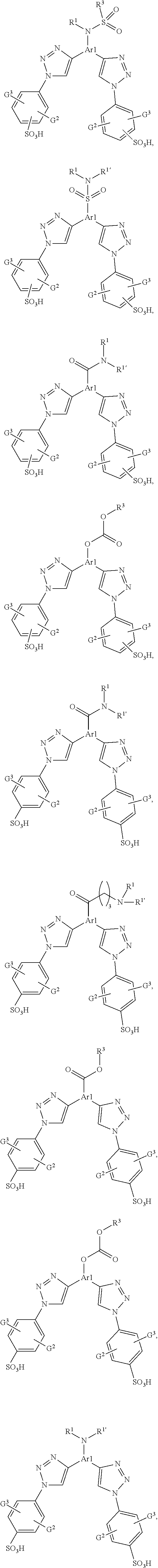

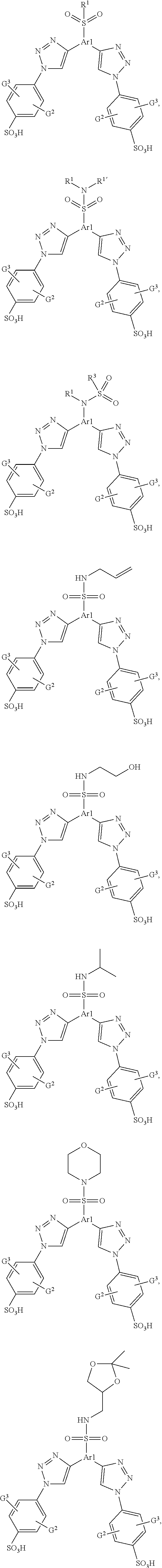

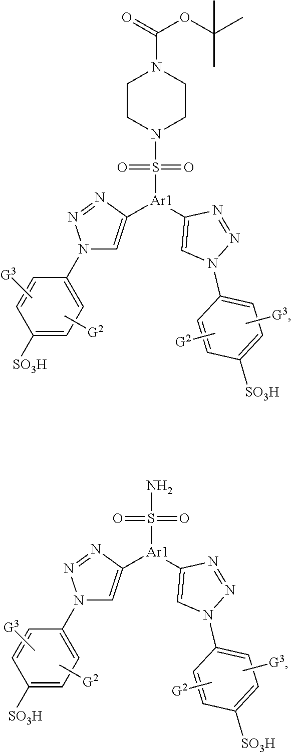

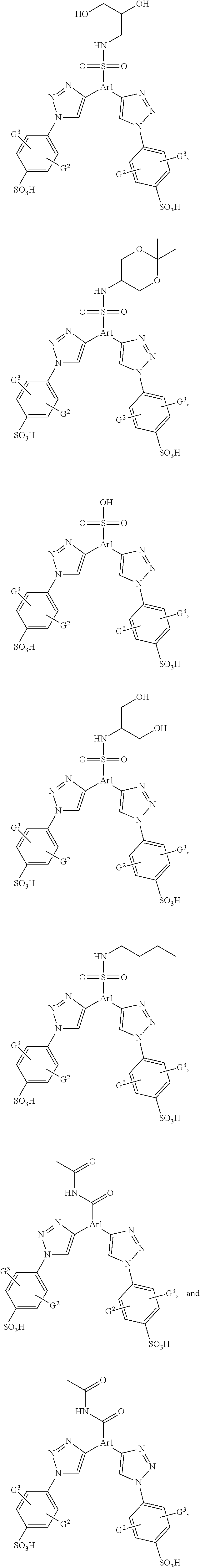

or a salt or hydrate thereof.
Embodiment 48) A composition comprising a compound of any one of embodiments 1-47, and a molecular crowding agent.
Embodiment 49) The composition of embodiment 48, wherein the molecular crowding agent is a polyalkylene glycol.
Embodiment 50) A composition comprising a compound of any one of embodiments 1-47, and an aqueous buffer.
Embodiment 51) The composition of embodiment 50, wherein the aqueous buffer is Tris HCL.
Embodiment 52) A composition comprising a compound of any one of embodiments 1-47, and a polynucleotide.
Embodiment 53) The composition of embodiment 52, wherein the polynucleotide is a 20-60 mer oligonucleotide.
Embodiment 54) The composition of embodiment 52, wherein the polynucleotide is a 25-50 mer oligonucleotide.
Embodiment 55) A composition comprising a compound of any one of embodiments 1-47, and a protein.
Embodiment 56) The composition of embodiment 55, wherein the protein is a DNA polymerase.
Embodiment 57) A composition comprising a compound of any one of embodiments 1-47, and a mixture of nucleotides or nucleotide analogs.
Embodiment 58) A composition for enhancing the processivity, fidelity, or rate of a DNA polymerase reaction comprising at least one compound of any one of embodiments 1-47, and a mixture of nucleotide analogs.
Embodiment 59) A composition comprising at least one compound of any one of embodiments 1-47 and a mixture of nucleotide analogs, wherein the at least one compound of any one of embodiments 1-47, increases the number and accuracy of nucleotide analogs incorporated into a daughter strand during a template-dependent polymerization reaction relative to an identical polymerization reaction absent the at least one compound of any one of embodiments 1-47.
Embodiment 60) The composition of embodiment 59, wherein the mixture of nucleotide analogs comprises nucleoside triphosphoramidates, wherein each of the nucleoside triphosphoramidates comprises a nucleobase selected from the group consisting of adenine, guanine, thymine, and cytosine and a polymeric tether moiety, wherein a first end of the polymeric tether moiety is attached to the nucleobase and a second end of the polymeric ether moiety is attached to the alpha phosphate of the nucleoside triphosphoramidate to provide for expansion of the nucleotide analogs by cleavage of the phosphoramidate bond.
Embodiment 61) The composition of embodiment 59, further comprising a buffer which includes one or more components selected from MnCl₂, a buffer, a salt, a sugar, a single-strand binding protein (SSB), imidazole, pyrazole, triazole, betaine, a molecular crowding agent, dimethyl sulfoxide (DMSO), an alkanediol, glycerol, N-methyl-2-pyrrolidone (NMP), acetamide, butylated hydroxyanisole (BHA), a polyphosphate, and/or urea.
Embodiment 62) The composition of embodiment 59, further comprising a single-strand binding protein.
Embodiment 63) The composition of embodiment 59, further comprising urea.
Embodiment 64) The composition of embodiment 59, wherein the mixture of nucleotide analogs comprises nucleotide analogs comprising a detectable label.
Embodiment 65) The composition of 64, wherein the detectable label is an optically detectable label selected from the group consisting of luminescent, chemiluminescent, fluorescent, fluorogenic, chromophoric or chromogenic labels.
Embodiment 66) A kit for sequencing a nucleic acid template comprising at least one composition of any one of embodiments 48-65.
Embodiment 67) A method of enhancing a nucleic acid polymerase reaction, the method comprising:
   a. forming a nucleic acid polymerase reaction composition comprising:
      i. a template nucleic acid,
      ii. a nucleic acid polymerase,
      iii. a mixture of nucleotides or nucleotide analogs, and
      iv. at least one compound of any of embodiments 1-47; and
   b. incubating the nucleic acid polymerase reaction composition under conditions allowing a nucleic acid polymerization reaction, wherein the at least one compound of any one of embodiments 1-47 increases the processivity, rate, or fidelity of the nucleic acid polymerase reaction.
Embodiment 68) The method of embodiment 67, wherein the compound of any one of embodiments 1-47 increases the length of a resulting nucleic acid product compared to a nucleic acid polymerase reaction lacking the compound of any one of embodiments 1-47.
Embodiment 69) The method of embodiment 67, wherein the at least one compound of any one of embodiments 1-47 comprises a plurality of compounds of any one of embodiments 1-47.
Embodiment 70) The method of embodiment 67, wherein the nucleic acid polymerase is a polymerase.
Embodiment 71) The method of embodiment 67, wherein the nucleic acid polymerase is a DNA polymerase.
Embodiment 72) The method of embodiment 71, wherein the DNA polymerase is DP04 or a variant thereof.
Embodiment 73) The method of embodiment 67, wherein the mixture of nucleotides or nucleotide analogs is a mixture of nucleotide analogs comprising nucleoside triphosphoramidates, wherein each of the nucleoside triphosphoramidates comprises a nucleobase selected from the group consisting of adenine, guanine, thymine, and cytosine and a polymeric tether moiety, wherein a first end of the polymeric tether moiety is attached to the nucleobase and a second end of the polymeric tether moiety is attached to the alpha phosphate of the nucleoside triphosphoramidate to provide for expansion of the nucleotide analogs by cleavage of the phosphoramidate bond.
Embodiment 74) The method of embodiment 67, wherein the nucleic acid polymerization reaction produces an expandable polymer of nucleotide analogs, wherein the expandable polymer encodes the nucleobase sequence information of the template nucleic acid.
Embodiment 75) The method of embodiment 67, wherein the conditions for facilitating a nucleic acid polymerization reaction comprise a suitable polymerization buffer and an oligonucleotide primer.
Embodiment 76) The method of embodiment 67, wherein the suitable buffer includes one or more components selected from MnCl₂, a buffer, a salt, a sugar, a single-strand binding protein (SSB), imidazole, pyrazole, triazole, betaine, a molecular crowding agent, dimethyl sulfoxide (DMSO), an alkanediol, glycerol, N-methyl-2-pyrrolidone (NMP), acetamide, butylated hydroxyanisole (BHA), a polyphosphate, and/or urea.
Embodiment 77) The method of embodiment 67, wherein the reaction mixture further comprises a single-strand binding protein.
Embodiment 78) The method of embodiment 67, wherein the reaction mixture further comprises urea.
Embodiment 79) The method of embodiment 67, wherein the mixture of nucleotides or nucleotide analogs comprises nucleotide analogs comprising a detectable label.
Embodiment 80) The method of embodiment 73, wherein the detectable label is an optically detectable label selected from the group consisting of luminescent, chemiluminescent, fluorescent, fluorogenic, chromophoric or chromogenic labels.
Embodiment 81) A method of sequencing a DNA or RNA template, the method comprising the steps of:
   a. forming a DNA polymerase reaction composition comprising:
      i. a DNA or RNA template,
      ii. a replication primer that complexes with the template,
      iii. a DNA polymerase,
      iv. a mixture of nucleotides or nucleotide analogs,
      v. at least one compound of any of embodiments 1-43,
   b. incubating the DNA polymerase reaction composition under conditions allowing a DNA polymerization reaction, wherein the at least one compound of any one of embodiments 1-47 increases the rate, fidelity or processivity of the DNA polymerase reaction; and
   c. determining the sequence of the nucleotides or nucleotide analogs in the resulting polymer of nucleotides or nucleotide analogs.
Embodiment 82) The method of embodiment 81, wherein the mixture of nucleotide analogs comprises nucleoside triphosphoramidates, wherein each of the nucleoside triphosphoramidates comprises a nucleobase selected from the group consisting of adenine, guanine, thymine, and cytosine and a polymeric tether moiety, wherein a first end of the polymeric tether moiety is attached to the nucleobase and a second end of the polymeric ether moiety is attached to the alpha phosphate of the nucleoside triphosphoramidate to provide for expansion of the nucleotide analogs by cleavage of the phosphoramidate bond.
Embodiment 83) The method of any one of embodiments 81 or 82, wherein the DNA polymerase is DP04 or a variant thereof.
Embodiment 84) The method of any one of embodiments 81 or 82, wherein the resulting polymer of nucleotide analogs is an expandable polymer.
Embodiment 85) The method of any one of embodiment 81, further including the step of contacting the expandable polymer with a phosphoramidate cleavage agent to produce an expanded polymer of nucleotide analogs.
Embodiment 86) The method of any one of embodiments 81 or 82, wherein the polymeric tether moiety of each of the nucleotide analogs comprises a reporter moiety unique to the nucleobase of the analog.
Embodiment 87) The method of embodiment 86, wherein the reporter moieties produce a characteristic electronic signal.
Embodiment 88) The method of embodiment 81, wherein the step of determining the sequence of the nucleotide analogs comprises the step of translocating the expanded polymer of nucleotide analogs through a nanopore.
Embodiment 89) A polymer, copolymer, or oligomer derived from one or more of the compounds of any one of embodiments 1-47.
Embodiment 90) An composition comprising one or more of the compounds of any one of embodiments 1-47.
Embodiment 91) The composition of embodiment 90, wherein the composition comprises at least two molecules of a compound having any one of embodiments 1-47.
Embodiment 92) A compound having the structure:

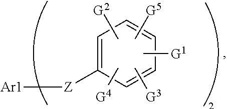

wherein
   a is 0 or an integer ranging from 1-4;
   a′ is 0 or an integer ranging from 1-4;
   Z is a 5-membered or 6-membered heteroaromatic ring including one, two, three, or four heteroatoms selected from O, N, or S;
   Ar1 is, at each occurrence, independently selected from optionally substituted phenyl, pyridine, bipyridine, tripyridine, pyrazine, pyridazine, furan, dibenzofuran, thiophene, pyrrole, selenophene, naphthalene, fluorene, phenanthrene, cinnoline, phthalazine, quinazoline, quinoxaline, naphthyridine, phenanthroline, purine, and carbazole,
   wherein substituents for Ar1 are, at each occurrence, independently selected from halogen, —OH, —CN, —NO₂, C₁-C₆ alkyl, C₁-C₆ haloalkyl, C₁-C₆ heteroalkyl, C₁-C₆ cycloloalkyl, —OR⁰, —CONH₂, —C(O)NR¹R^{1′}, —C(O)(CH₂)ₐNR¹R^{1′}, —NR¹R^{1′}, —NR¹C(O)R³, —C(O)SR³, —COR³, —CO(CH₂)ₐOC(O)R³, —OC(O)R³, —C(O)OR³, —C—O—R³, mercaptan, —R⁴—H, —SOR¹, —S(O)₂R¹, —S(O)₂NR¹R^{1′}, —CH₂—NR¹S(O)₂R³, —NR¹S(O)₂R³, and —C(CH₃)═N-(phenyl)-O—CH₂—C—CH;
   R⁰ is, at each occurrence, independently selected from C₁-C₆ alkyl, C₁-C₆ haloalkyl, C₂-C₆ alkenyl, C₂-C₆ alkynyl, substituted or unsubstituted cycloalkyl, substituted or unsubstituted heterocyclyl, substituted or unsubstituted aryl, and substituted or unsubstituted heteroaryl;
   R¹ and R^{1′} are, at each occurrence, independently selected from H, hydroxyl, C₁-C₆ alkyl, C₁-C₆ haloalkyl, C₁-C₂₀ heteroalkyl, C₁-C₁₀ heteroalkyl-NH₂, C₂-C₆ alkenyl, C₂-C₆ alkynyl, substituted or unsubstituted cycloalkyl, substituted or unsubstituted heterocyclyl (e.g., substituted with one or more R³ groups), substituted or unsubstituted aryl, substituted or unsubstituted arylalkyl, and substituted or unsubstituted heteroaryl, benzyl, —C(═NH)NH₂, —CH₂CO₂R⁰, —C(H)(CHₐ(COOH))₂, —CH₂C(O)NHCH₂CO₂H, —CH₂CH₂OH, —CH₂CH₂NHC(O)R³, —(CH₂)ₐC(O)NH(CH₂)ₐC(O)OR³, —(CH₂)ₐ—CN, spermine, —(CH₂)ₐ—NH₂, —C(O)R³, —(CH₂)ₐC(O)OR³, —(CH₂)ₐOR³, —C(H)((CH₂)ₐOH)_{a′}, —CHCH(OH)(CH₂)ₐOH, —(CH₂)ₐOH, C₁-C₆—C(O)OH, —(CH₂)ₐ-heterocycle which may be substituted (e.g., such as with an R³ group) or unsubstituted,

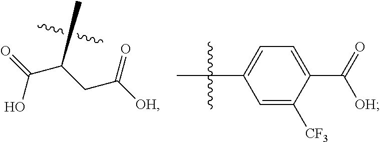

and
wherein R¹ and R^{1′} together form a substituted or unsubstituted heterocyclic ring, including, but not limited to, azetidine, pyrrolidine, piperidine, piperazine, morpholine,

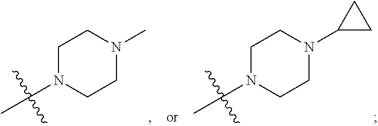

R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine;
R⁴ is, at each occurrence, independently selected from one or more heteroatom interrupted alkylene, wherein the heteroatom is O, S, NH, or a combination thereof, G¹ is, at each occurrence, independently selected from oxo, C₁-C₆ alkyl, C₁-C₆ haloalkyl, -E-O—R³, -E-C(R¹)(R^{1′})(R³), —NH₂, —NO₂, —SO₃R³, —SO₃O⁻, —SO₃N(H)(R¹), -E-C(O)R³, -E-CO₂H, —B(OH)₂, —C(O)NR¹R^{1′}, -E-PO(OR¹)₂, and aryl substituted with G², G³, G⁴ and G⁵; and
G², G³, G⁴ and G⁵ are, at each occurrence, independently selected from absent or selected from the groups comprising, halogen, —CN, —NO₂, —SO₃R³, C₁-C₆ alkyl, C₁-C₆ haloalkyl, -E-O—R³, -E-(CH₂)ₐC(O)R³, -E-CO₂H, -E-CHO, -E-C(O)R³, -E-C(O)NH(OH), -E-C(O)NHR¹, -E-C(O)N(H)C(H)(R¹)(R^{1′}), -E-C(O)N(R¹)((CH₂)ₐP(O)(O)OH)₂, -E-C(O)N(R¹)((CH₂)ₐSO₃H), -E-C(O)NR¹R^{1′}, -E-NR¹R^{1′}, -E-OR², —C(O)-heterocycloalkyl (where the heterocycloalkyl is substituted or unsubstituted), —C(O)—N(H)—CH(COOH)((CH₂)ₐ-heteroaryl) (where heteroaryl is substituted or unsubstituted), wherein E is, at each occurrence, independently selected from a direct bond, and C₁-C₆ alkylene.
Embodiment 93) The compound of embodiment 92, wherein each substituent of Ar1 is independently selected from—C(O)—CH₃, —C(O)—NH₂, —C(O)O—CH₂CH₃, —CF₃, —C(O)O-t-butyl, —C(O)—N(CH₃)₂, —C(O)—N(H)CH₃, —C(O)—N(H)CH₂CH₃, —C(O)—N(H)(cycloalkyl), —C(O)—N(H)(CH₂)₄, —C(O)—N(CH₂CH₃)₂, —C(O)—N(H)(t-butyl), —C(O)—N(H)(C₆-cycloalkyl), —C(O)—N(H)(CH₂)₃, —C(O)—N(H)(C₅-cycloalkyl), —C(O)—N(H)(C₄-cycloalkyl), —C(O)—N(H)(phenyl), —C(O)—N(H)(CH₂CH₂)N(H)C(O)—CH₃, —C(O)—N(H)C(NH)(NH₂), —C(O)—N(CH₂CH₂CH₃)₂, —C(O)—N—(C(H)(CH₃)(CH₂CH₃))₂, —C(O)—N(CH₂CH₂CH₂CH₃)₂, —C(O)—N(H)(benzyl), —C(O)—N(H)—CH₂CH₂OH, —C(O)—S—CH₂CH₃, —C(O)N(CH₂)₂, —N(H)S(O)(O)-phenyl, —C(O)N(H)CH₂CH₂CF₃, —C(O)N(H)CH₃, —C(O)OCH₂CH₃, —C(O)—N(H)—CH₂CH₂O—CH₂CH₂O—CH₃, —C(O)—N(H)—(CH₂CH₂O)₈—CH₃, —C(O)N(H)—CH₂—CH₂—CH₂—N(H)—CH₂—CH₂—CH₂—N(H)—CH₂—CH₂—CH₂—NH₂, —C(O)N(H)—CH₂—CH₂—NH₂, —C(O)N(H)C(O)CH₃, —C(O)N(H)—CH₂—CH₃, —S(O)(O)N(H)—CH₂—CH₃, —S(O)(O)N—(CH₂—CH₃)₂, —S(O)(O)N(H)—(C₃-cycloalkyl), —S(O)(O)N(H)—(CH₂CH₂)OH, —S(O)(O)OH, —C(O)N(H)C(H)(CH₂OH)₂, —C(O)N(CH₂CH₂OH)₂, —C(O)N(H)CH₂CH₂═CH₂, —C(O)CH₂N(CH₃)(CH₂)(COOH), —S(O)(O)(CH₂CH₃), —S(O)(O)N(H)CH₂—CH(OH)(CH₂OH), —S(O)(O)—NH₂, and —S(O)(O)N(H)CH₂—C(O)(O-t-butyl).
Embodiment 94) A compound having the structure:

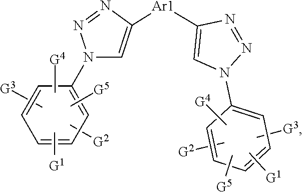

a is 0 or an integer ranging from 1-4;
a′ is 0 or an integer ranging from 1-4;
Ar1 is, at each occurrence, independently selected from optionally substituted phenyl, pyridine, bipyridine, tripyridine, pyrazine, pyridazine, furan, dibenzofuran, thiophene, pyrrole, selenophene, naphthalene, fluorene, phenanthrene, cinnoline, phthalazine, quinazoline, quinoxaline, naphthyridine, phenanthroline, purine, and carbazole,
wherein substituents for Ar1 are, at each occurrence, independently selected from from—C(O)—CH₃, —C(O)—NH₂, —C(O)O—CH₂CH₃, —CF₃, —C(O)O-t-butyl, —C(O)—N(CH₃)₂, —C(O)—N(H)CH₃, —C(O)—N(H)CH₂CH₃, —C(O)—N(H)(cycloalkyl), —C(O)—N(H)(CH₂)₄, —C(O)—N(CH₂CH₃)₂, —C(O)—N(H)(t-butyl), —C(O)—N(H)(C₆-cycloalkyl), —C(O)—N(H)(CH₂)₃, —C(O)—N(H)(C₅-cycloalkyl), —C(O)—N(H)(C₄-cycloalkyl), —C(O)—N(H)(phenyl), —C(O)—N(H)(CH₂CH₂)N(H)C(O)—CH₃, —C(O)—N(H)C(NH)(NH₂), —C(O)—N(CH₂CH₂CH₃)₂, —C(O)—N—(C(H)(CH₃)(CH₂CH₃))₂, —C(O)—N(CH₂CH₂CH₂CH₃)₂, —C(O)—N(H)(benzyl), —C(O)—N(H)—CH₂CH₂OH, —C(O)—S—CH₂CH₃, —C(O)N(CH₂)₂, —N(H)S(O)(O)-phenyl, —C(O)N(H)CH₂CH₂CF₃, —C(O)N(H)CH₃, —C(O)OCH₂CH₃, —C(O)—N(H)—CH₂CH₂O—CH₂CH₂O—CH₃, —C(O)—N(H)—(CH₂CH₂O)₈—CH₃, —C(O)N(H)—CH₂—CH₂—CH₂—N(H)—CH₂—CH₂—CH₂—N(H)—CH₂—CH₂—CH₂—NH₂, —C(O)N(H)—CH₂—CH₂—NH₂, —C(O)N(H)C(O)CH₃, —C(O)N(H)—CH₂—CH₃, —S(O)(O)N(H)—CH₂—CH₃, —S(O)(O)N—(CH₂—CH₃)₂, —S(O)(O)N(H)—(C3-cycloalkyl), —S(O)(O)N(H)—(CH₂CH₂)OH, —S(O)(O)OH, —C(O)N(H)C(H)(CH₂OH)₂, —C(O)N(CH₂CH₂OH)₂, —C(O)N(H)CH₂CH₂═CH₂, —C(O)CH₂N(CH₃)(CH₂)(COOH), —S(O)(O)(CH₂CH₃), —S(O)(O)N(H)CH₂—CH(OH)(CH₂OH), —S(O)(O)—NH₂, —S(O)(O)N(H)CH₂—C(O)(O-t-butyl);
   and wherein G¹, G², G³, G⁴, and G⁵ are as defined herein.
R⁰ is, at each occurrence, independently selected from C₁-C₆ alkyl, C₁-C₆ haloalkyl, C₂-C₆ alkenyl, C₂-C₆ alkynyl, substituted or unsubstituted cycloalkyl, substituted or unsubstituted heterocyclyl, substituted or unsubstituted aryl, and substituted or unsubstituted heteroaryl;
R¹ and R^{1′} are, at each occurrence, independently selected from H, hydroxyl, C₁-C₆ alkyl, C₁-C₆ haloalkyl, C₁-C₂₀ heteroalkyl, C₁-C₁₀ heteroalkyl-NH₂, C₂-C₆ alkenyl, C₂-C₆ alkynyl, substituted or unsubstituted cycloalkyl, substituted or unsubstituted heterocyclyl (e.g., substituted with one or more R³ groups), substituted or unsubstituted aryl, substituted or unsubstituted arylalkyl, and substituted or unsubstituted heteroaryl, benzyl, —C(═NH)NH₂, —CH₂CO₂R⁰, —C(H)(CHₐ(COOH))₂, —CH₂C(O)NHCH₂CO₂H, —CH₂CH₂OH, —CH₂CH₂NHC(O)R³, —(CH₂)ₐC(O)NH(CH₂)ₐC(O)OR³, —(CH₂)ₐ—CN, spermine, —(CH₂)ₐ—NH₂, —C(O)R³, —(CH₂)ₐC(O)OR³, —(CH₂)ₐOR³, —C(H)((CH₂)ₐOH)ₐ,, —CHCH(OH)(CH₂)ₐOH, —(CH₂)ₐOH, C₁-C₆—C(O)OH, —(CH₂)ₐ-heterocycle which may be substituted (e.g., such as with an R³ group) or unsubstituted,

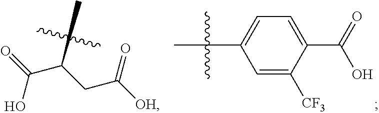

and
wherein R¹ and R^{1′} together form a substituted or unsubstituted heterocyclic ring, including, but not limited to, azetidine, pyrrolidine, piperidine, piperazine, morpholine,

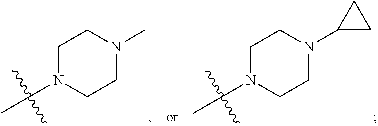

R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine;
R⁴ is, at each occurrence, independently selected from one or more heteroatom interrupted alkylene, wherein the heteroatom is O, S, NH, or a combination thereof;
G¹ is, at each occurrence, independently selected from oxo, C₁-C₆ alkyl, C₁-C₆ haloalkyl, -E-O—R³, -E-C(R¹)(R^{1′})(R³), —NH₂, —NO₂, —SO₃R³, —SO₃O⁻, —SO₃N(H)(R¹), -E-C(O)R³, -E-CO₂H, —B(OH)₂, —C(O)NR¹R^{1′}, -E-PO(OR¹)₂, and aryl substituted with G², G³, G⁴ and G⁵; and
G², G³, G⁴ and G⁵ are, at each occurrence, independently selected from absent or selected from the groups comprising, halogen, —CN, —NO₂, —SO₃R³, C₁-C₆ alkyl, C₁-C₆ haloalkyl, -E-O—R³, -E-(CH₂)ₐC(O)R³, -E-CO₂H, -E-CHO, -E-C(O)R³, -E-C(O)NH(OH), -E-C(O)NHR¹, -E-C(O)N(H)C(H)(R¹)(R^{1′}), -E-C(O)N(R¹)((CH₂)ₐP(O)(O)OH)₂, -E-C(O)N(R)((CH₂)ₐSO₃H), -E-C(O)NR¹R^{1′}, -E-NR¹R^{1′}, -E-OR², —C(O)-heterocycloalkyl (where the heterocycloalkyl is substituted or unsubstituted), —C(O)—N(H)—CH(COOH)((CH₂)ₐ-heteroaryl) (where heteroaryl is substituted or unsubstituted), wherein E is, at each occurrence, independently selected from a direct bond, and C₁-C₆ alkylene.
Embodiment 95) A compound having any one of Formulas (IB) or (ID).

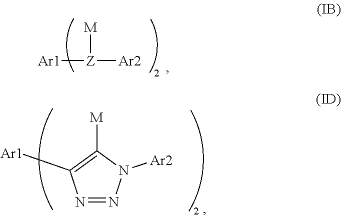

wherein
wherein
   a is 0 or an integer ranging from 1-4;
   a′ is 0 or an integer ranging from 1-4;
   Z is a 5-membered or 6-membered heteroaromatic ring including one, two, three, or four heteroatoms selected from O, N, or S;
   M is, at each occurrence, independently selected from hydrogen, halogen and C₁-C₄ alkyl;
   Ar1 is, at each occurrence, independently selected from optionally substituted phenyl, pyridine, bipyridine, tripyridine, pyrazine, pyridazine, furan, dibenzofuran, thiophene, pyrrole, selenophene, naphthalene, fluorene, phenanthrene, cinnoline, phthalazine, quinazoline, quinoxaline, naphthyridine, phenanthroline, purine, and carbazole,
   wherein substituents for Ar1 are, at each occurrence, independently selected from halogen, —OH, —CN, —NO₂, C₁-C₆ alkyl, C₁-C₆ haloalkyl, C₁-C₆ heteroalkyl, C₁-C₆ cycloloalkyl, —OR⁰, —CONH₂, —C(O)NR¹R^{1′}, —C(O)(CH₂)ₐNR¹R^{1′}, —NR¹R^{1′}, —NR¹C(O)R³, —C(O)SR³, —COR³, —CO(CH₂)ₐOC(O)R³, —OC(O)R³, —C(O)OR³, —C—O—R³, mercaptan, —R⁴—H, —SOR¹, —S(O)₂R¹, —S(O)₂NR¹R^{1′}, —CH₂—NR¹S(O)₂R³, —NR¹S(O)₂R³, and —C(CH₃)═N-(phenyl)-O—CH₂—C—CH;
   R⁰ is, at each occurrence, independently selected from C₁-C₆ alkyl, C₁-C₆ haloalkyl, C₂-C₆ alkenyl, C₂-C₆ alkynyl, substituted or unsubstituted cycloalkyl, substituted or unsubstituted heterocyclyl, substituted or unsubstituted aryl, and substituted or unsubstituted heteroaryl;
   R¹ and R^{1′} are, at each occurrence, independently selected from H, hydroxyl, C₁-C₆ alkyl, C₁-C₆ haloalkyl, C₁-C₂₀ heteroalkyl, C₁-C₁₀ heteroalkyl-NH₂, C₂-C₆ alkenyl, C₂-C₆ alkynyl, substituted or unsubstituted cycloalkyl, substituted or unsubstituted heterocyclyl (e.g., substituted with one or more R³ groups), substituted or unsubstituted aryl, substituted or unsubstituted arylalkyl, and substituted or unsubstituted heteroaryl, benzyl, —C(═NH)NH₂, —CH₂CO₂R⁰, —C(H)(CHₐ(COOH))₂, —CH₂C(O)NHCH₂CO₂H, —CH₂CH₂OH, —CH₂CH₂NHC(O)R³, —(CH₂)ₐC(O)NH(CH₂)ₐC(O)OR³, —(CH₂)ₐ—CN, spermine, —(CH₂)ₐ—NH₂, —C(O)R³, —(CH₂)ₐC(O)OR³, —(CH₂)ₐOR³, —C(H)((CH₂)ₐOH)_{a′}, —CHCH(OH)(CH₂)ₐOH, —(CH₂)ₐOH, C₁-C₆—C(O)OH, —(CH₂)ₐ-heterocycle which may be substituted (e.g., such as with an R³ group) or unsubstituted,

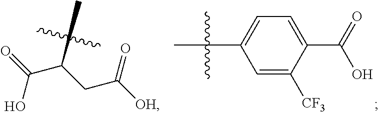

wherein R¹ and R^{1′} together form a substituted or unsubstituted heterocyclic ring, including, but not limited to, azetidine, pyrrolidine, piperidine, piperazine, morpholine,

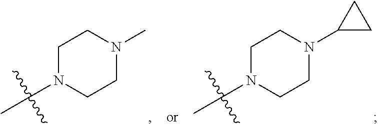

R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine;
R⁴ is, at each occurrence, independently selected from one or more heteroatom interrupted alkylene, wherein the heteroatom is O, S, NH, or a combination thereof,
Ar2 is, at each occurrence, independently selected from (i) a substituted or unsubstituted 5- and 6-membered monocyclic aromatic or heteroaromatic ring; (ii) substituted 9- and 10-membered fused bicyclic rings comprising two monocyclic rings a substituted or unsubstituted, where at least one of the two monocyclic rings is an aromatic or a heteroaromatic ring; and (iii) a substituted or unsubstituted 13- and 14-membered fused tricyclic rings comprising three monocyclic rings together, where at least one of the three monocyclic rings is an aromatic or a heteroaromatic ring;
wherein each Ar2 is independently substituted with G¹, G², G³, G⁴ and G⁵, wherein:
when Ar2 is monosubstituted, G¹ is, at each occurrence, independently selected from oxo, C₁-C₆ alkyl, C₁-C₆ haloalkyl, -E-O—R³, -E-C(R¹)(R^{1′})(R³), —NH₂, —NO₂, —SO₃R³, —SO₃O⁻, —SO₃N(H)(R′), -E-C(O)R³, -E-CO₂H, —B(OH)₂, —C(O)NR¹R^{1′}, -E-PO(OR¹)₂, and aryl substituted with G², G³, G⁴ and G⁵;
G², G³, G⁴ and G⁵ are, at each occurrence, independently selected from absent or selected from the groups comprising, halogen, —CN, —NO₂, —SO₃R³, C₁-C₆ alkyl, C₁-C₆ haloalkyl, -E-O—R³, -E-(CH₂)ₐC(O)R³, -E-CO₂H, -E-CHO, -E-C(O)R³, -E-C(O)NH(OH), -E-C(O)NHR¹, -E-C(O)N(H)C(H)(R¹)(R^{1′}), -E-C(O)N(R¹)((CH₂)ₐP(O)(O)OH)₂, -E-C(O)N(R¹)((CH₂)ₐSO₃H), -E-C(O)NR¹R^{1′}, -E-NR¹R^{1′}, -E-OR², —C(O)-heterocycloalkyl (where the heterocycloalkyl is substituted or unsubstituted), —C(O)—N(H)—CH(COOH)((CH₂)ₐ-heteroaryl) (where heteroaryl is substituted or unsubstituted), wherein E is, at each occurrence, independently selected from a direct bond and C₁-C₆ alkylene.
Embodiment 96) A composition comprising two or more molecules of a compound having any one of the following formulas:

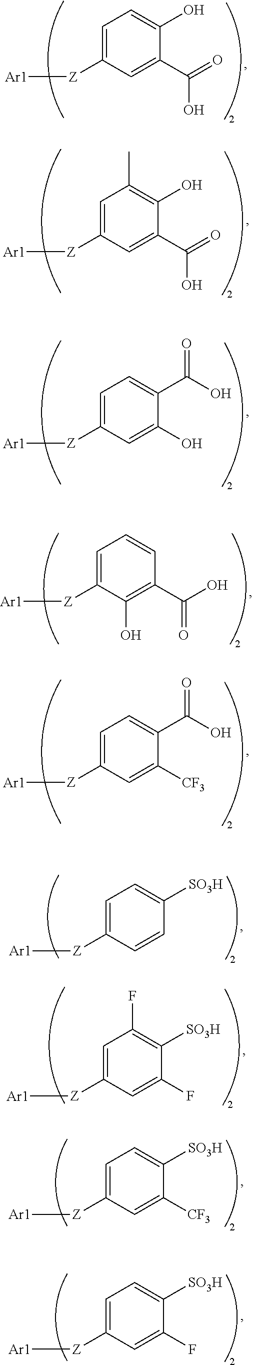

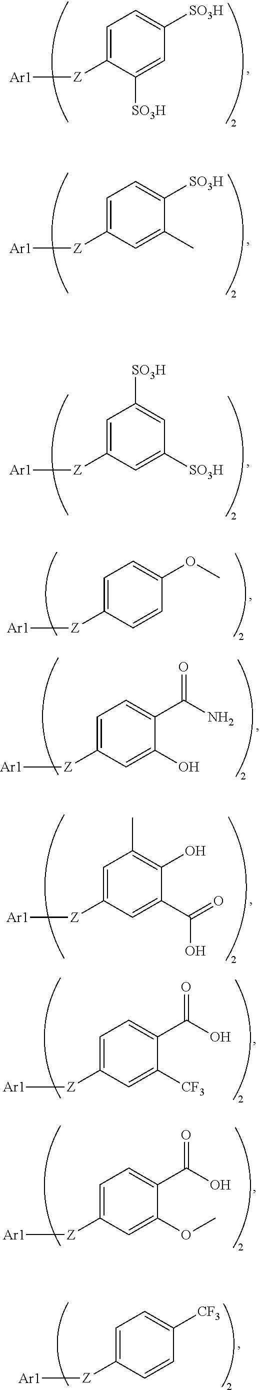

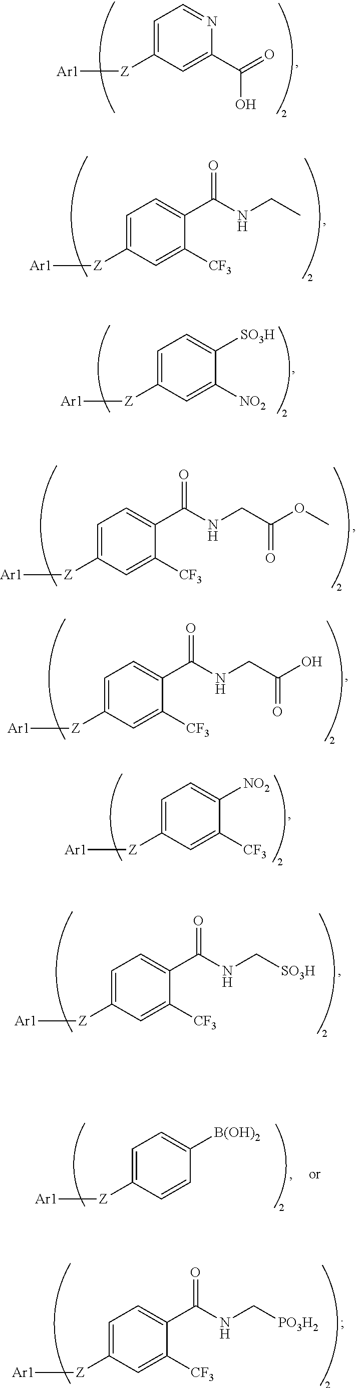

wherein
a is 0 or an integer ranging from 1-4;
a′ is 0 or an integer ranging from 1-4;
Z is a 5-membered or 6-membered heteroaromatic ring including one, two, three, or four heteroatoms selected from O, N, or S;
Ar1 is, at each occurrence, independently selected from optionally substituted phenyl, pyridine, bipyridine, tripyridine, pyrazine, pyridazine, furan, dibenzofuran, thiophene, pyrrole, selenophene, naphthalene, fluorene, phenanthrene, cinnoline, phthalazine, quinazoline, quinoxaline, naphthyridine, phenanthroline, purine, and carbazole,
wherein substituents for Ar1 are, at each occurrence, independently selected from halogen, —OH, —CN, —NO₂, C₁-C₆ alkyl, C₁-C₆ haloalkyl, C₁-C₆ heteroalkyl, C₁-C₆ cycloloalkyl, —OR⁰, —CONH₂, —C(O)NR¹R^{1′}, —C(O)(CH₂)ₐNR¹R^{1′}, —NR¹R^{1′}, —NR¹C(O)R³, —C(O)SR³, —COR³, —CO(CH₂)ₐOC(O)R³, —OC(O)R³, —C(O)OR³, —C—O—R³, mercaptan, —R⁴—H, —SOR¹, —S(O)₂R¹, —S(O)₂NR¹R^{1′}, —CH₂—NR¹S(O)₂R³, —NR¹S(O)₂R³, and —C(CH₃)═N-(phenyl)-O—CH₂—C—CH;
R⁰ is, at each occurrence, independently selected from C₁-C₆ alkyl, C₁-C₆ haloalkyl, C₂-C₆ alkenyl, C₂-C₆ alkynyl, substituted or unsubstituted cycloalkyl, substituted or unsubstituted heterocyclyl, substituted or unsubstituted aryl, and substituted or unsubstituted heteroaryl;
R¹ and R^{1′} are, at each occurrence, independently selected from H, hydroxyl, C₁-C₆ alkyl, C₁-C₆ haloalkyl, C₁-C₂₀ heteroalkyl, C₁-C₁₀ heteroalkyl-NH₂, C₂-C₆ alkenyl, C₂-C₆ alkynyl, substituted or unsubstituted cycloalkyl, substituted or unsubstituted heterocyclyl (e.g., substituted with one or more R³ groups), substituted or unsubstituted aryl, substituted or unsubstituted arylalkyl, and substituted or unsubstituted heteroaryl, benzyl, —C(═NH)NH₂, —CH₂CO₂R⁰, —C(H)(CHₐ(COOH))₂, —CH₂C(O)NHCH₂CO₂H, —CH₂CH₂OH, —CH₂CH₂NHC(O)R³, —(CH₂)ₐC(O)NH(CH₂)ₐC(O)OR³, —(CH₂)ₐ—CN, spermine, —(CH₂)ₐ—NH₂, —C(O)R³, —(CH₂)ₐC(O)OR³, —(CH₂)ₐOR³, —C(H)((CH₂)ₐOH)_{a′}, —CHCH(OH)(CH₂)ₐOH, —(CH₂)ₐOH, C₁-C₆—C(O)OH, —(CH₂)ₐ-heterocycle which may be substituted (e.g., such as with an R³ group) or unsubstituted,

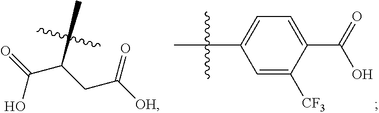

and
wherein R¹ and R^{1′} together form a substituted or unsubstituted heterocyclic ring, including, but not limited to, azetidine, pyrrolidine, piperidine, piperazine, morpholine,

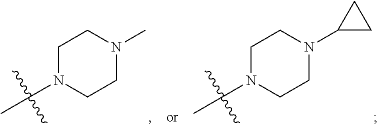

R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine; and
R⁴ is, at each occurrence, independently selected from one or more heteroatom interrupted alkylene, wherein the heteroatom is O, S, NH, or a combination thereof.
Embodiment 97) The compound of embodiment 96, wherein Z is selected from imidazole, pyrazole, pyrimidine, pyridazine, pyrazine, pyrrole, thiophene, triazole (e.g., 1,2,3-triazole; 1,2,4-triazole, thiazole, and.
Embodiment 98) The compound of embodiment 96, where each Z includes one, two, or three “M” moieties.
Embodiment 99) The compound of embodiment 96, wherein Ar1 is substituted with —C(O)—CH₃, —C(O)—NH₂, —C(O)O—CH₂CH₃, —CF₃, —C(O)O-t-butyl, —C(O)—N(CH₃)₂, —C(O)—N(H)CH₃, —C(O)—N(H)CH₂CH₃, —C(O)—N(H)(cycloalkyl), —C(O)—N(H)(CH₂)₄, —C(O)—N(CH₂CH₃)₂, —C(O)—N(H)(t-butyl), —C(O)—N(H)(C₆-cycloalkyl), —C(O)—N(H)(CH₂)₃, —C(O)—N(H)(C₅-cycloalkyl), —C(O)—N(H)(C₄-cycloalkyl), —C(O)—N(H)(phenyl), —C(O)—N(H)(CH₂CH₂)N(H)C(O)—CH₃, —C(O)—N(H)C(NH)(NH₂), —C(O)—N(CH₂CH₂CH₃)₂, —C(O)—N—(C(H)(CH₃)(CH₂CH₃))₂, —C(O)—N(CH₂CH₂CH₂CH₃)₂, —C(O)—N(H)(benzyl), —C(O)—N(H)—CH₂CH₂OH, —C(O)—S—CH₂CH₃, —C(O)N(CH₂)₂, —N(H)S(O)(O)-phenyl, —C(O)N(H)CH₂CH₂CF₃, —C(O)N(H)CH₃, —C(O)OCH₂CH₃, —C(O)—N(H)—CH₂CH₂O—CH₂CH₂O—CH₃, —C(O)—N(H)—(CH₂CH₂O)₈—CH₃, —C(O)N(H)—CH₂—CH₂—CH₂—N(H)—CH₂—CH₂—CH₂—N(H)—CH₂—CH₂—CH₂—NH₂, —C(O)N(H)—CH₂—CH₂—NH₂, —C(O)N(H)C(O)CH₃, —C(O)N(H)—CH₂—CH₃, —S(O)(O)N(H)—CH₂—CH₃, —S(O)(O)N—(CH₂—CH₃)₂, —S(O)(O)N(H)—(C3-cycloalkyl), —S(O)(O)N(H)—(CH₂CH₂)OH, —S(O)(O)OH, —C(O)N(H)C(H)(CH₂OH)₂, —C(O)N(CH₂CH₂OH)₂, —C(O)N(H)CH₂CH₂═CH₂, —C(O)CH₂N(CH₃)(CH₂)(COOH), —S(O)(O)(CH₂CH₃), —S(O)(O)N(H)CH₂—CH(OH)(CH₂OH), —S(O)(O)—NH₂, or S(O)(O)N(H)CH₂—C(O)(O-t-butyl).
Embodiment 100) A compound having Formula (I):

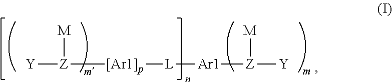

or a solvate, hydrate, tautomer, chelate or salt thereof,
wherein
   a is 0 or an integer ranging from 1-4;
   a′ is 0 or an integer ranging from 1-4;
   m is 1, 2 or 3;
   m′ is 1, 2 or 3;
   n is 0, 1 or 2;
   p is 0, 1 or 2;
   Z is a 5-membered or 6-membered heteroaromatic ring including one, two, three, or four heteroatoms selected from O, N, or S;
   Y is, at each occurrence, independently selected from Ar2, —C(O)—Ar2, —(CH₂)ₐAr2, —(CH₂)₃PO(OEt)₂, or —CH₂CO₂Me;
   L is a linking group;
   M is, at each occurrence, independently selected from hydrogen, halogen and C₁-C₄ alkyl;
      Ar1 is, at each occurrence, independently selected from optionally substituted phenyl, pyridine, bipyridine, tripyridine, pyrazine, pyridazine, furan, dibenzofuran, thiophene, pyrrole, selenophene, naphthalene, fluorene, phenanthrene, cinnoline, phthalazine, quinazoline, quinoxaline, naphthyridine, phenanthroline, purine, and carbazole,
      wherein substituents for Ar1 are, at each occurrence, independently selected from halogen, —OH, —CN, —NO₂, C₁-C₆ alkyl, C₁-C₆ haloalkyl, C₁-C₆ heteroalkyl, C₁-C₆ cycloalkyl, —OR⁰, —CONH₂, —C(O)NR¹R^{1′}, —C(O)(CH₂)ₐNR¹R^{1′}, —NR¹R^{1′}, —NR¹C(O)R³, —C(O)SR³, —COR³, —CO(CH₂)ₐOC(O)R³, —OC(O)R³, —C(O)OR³, —C—O—R³, mercaptan, —R⁴—H, —SOR¹, —S(O)₂R¹, —S(O)₂NR¹R^{1′}, —CH₂—NR'S(O)₂R³, —NR¹S(O)₂R³, and —C(CH₃)═N-(phenyl)-O—CH₂—C—CH;
      R⁰ is, at each occurrence, independently selected from C₁-C₆ alkyl, C₁-C₆ haloalkyl, C₂—C ₆alkenyl, C₂-C₆ alkynyl, substituted or unsubstituted cycloalkyl, substituted or unsubstituted heterocyclyl, substituted or unsubstituted aryl, and substituted or unsubstituted heteroaryl;
      R¹ and R^{1′} are, at each occurrence, independently selected from H, hydroxyl, C₁-C₆ alkyl, C₁-C₆ haloalkyl, C₁-C₂₀ heteroalkyl, C₁-C₁₀ heteroalkyl-NH₂, C₂-C₆ alkenyl, C₂-C₆ alkynyl, substituted or unsubstituted cycloalkyl, substituted or unsubstituted heterocyclyl, substituted or unsubstituted aryl, substituted or unsubstituted arylalkyl, and substituted or unsubstituted heteroaryl, benzyl, —C(═NH)NH₂, —CH₂CO₂R⁰, —C(H)(CHₐ(COOH))₂, —CH₂C(O)NHCH₂CO₂H, —CH₂(CH₂)ₐOH, —CH₂CH₂NHC(O)R³, —(CH₂)ₐC(O)NH(CH₂)ₐC(O)OR³, —(CH₂)ₐ—CN, spermine, —(CH₂)ₐ—NH₂, —C(O)R³, —(CH₂)ₐC(O)OR³, —(CH₂)ₐOR³, —C(H)((CH₂)ₐOH)_{a′}, —CHCH(OH)(CH₂)ₐOH, —(CH₂)ₐOH, C₁-C₆—C(O)OH, —(CH₂)ₐ-heterocycle which may be substituted or unsubstituted,

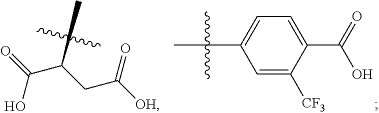

and
wherein R¹ and R^{1′} together form a substituted or unsubstituted heterocyclic ring, including, but not limited to, azetidine, pyrrolidine, piperidine, piperazine, morpholine,

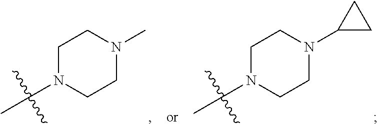

R² is, at each occurrence, independently selected from C₂-C₆ alkyl, C₁-C₆ haloalkyl, C₁-C₆ heteroalkyl, or substituted or unsubstituted cycloalkyl, substituted or unsubstituted heterocyclyl, substituted or unsubstituted aryl, substituted or unsubstituted heteroaryl, and substituted or unsubstituted haloalkoxy;
R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine;
R⁴ is, at each occurrence, independently selected from one or more heteroatom interrupted alkylene, wherein the heteroatom is O, S, NH, or a combination thereof,
Ar2 is, at each occurrence, independently selected from (i) a substituted or unsubstituted 5- and 6-membered monocyclic aromatic or heteroaromatic ring; (ii) a substituted or unsubstituted 9- and 10-membered fused bicyclic rings comprising two monocyclic rings together, where at least one of the two monocyclic rings is an aromatic or a heteroaromatic ring; and (iii) a substituted or unsubstituted 13- and 14-membered fused tricyclic rings comprising three monocyclic rings together, where at least one of the three monocyclic rings is an aromatic or a heteroaromatic ring;
wherein each Ar2 is independently substituted with G¹, G², G³, G⁴ and G⁵, wherein:
when Ar2 is monosubstituted, G¹ is, at each occurrence, independently selected from oxo, C₁-C₆ alkyl, C₁-C₆ haloalkyl, -E-O—R³, -E-C(R¹)(R^{1′})(R³), —NH₂, —NO₂, —SO₃R³, —SO₃O⁻, —SO₃N(H)(R′), -E-C(O)R³, -E-CO₂H, —B(OH)₂, —C(O)NR¹R^{1′}, -E-PO(OR¹)₂, and aryl substituted with G², G³, G⁴ and G⁵; and
G², G³, G⁴ and G⁵ are, at each occurrence, independently selected from absent or selected from the groups comprising, halogen, —CN, —NO₂, —SO₃R³, C₁-C₆ alkyl, C₁-C₆ haloalkyl, -E-O—R³, -E-(CH₂)ₐC(O)R³, -E-CO₂H, -E-CHO, -E-C(O)R³, -E-C(O)NH(OH), -E-C(O)NHR¹, -E-C(O)N(H)C(H)(R¹)(R^{1′}), -E-C(O)N(R¹)((CH₂)ₐP(O)(O)OH)₂, -E-C(O)N(R¹)((CH₂)ₐSO₃H), -E-C(O)NR¹R^{1′}, -E-NR¹R^{1′}, -E-OR², —C(O)-heterocycloalkyl (where the heterocycloalkyl is substituted or unsubstituted), —C(O)—N(H)—CH(COOH)((CH₂)ₐ-heteroaryl) (where heteroaryl is substituted or unsubstituted), wherein E is, at each occurrence, independently selected from a direct bond, and C₁-C₆ alkylene;
wherein Z is not a triazole.
Embodiment 101) The compound of embodiment 100, wherein Z includes two heteroatoms.
Embodiment 102) The compound of embodiment 100, wherein Z is a diazole.
Embodiment 103) The compound of embodiment 102, wherein the diazole is selected from the group consisting of imidazole, pyrazole, pyrimidine, pyridazine, and pyrazine.
Embodiment 104) The compound of embodiment 100, wherein Z includes three heteroatoms.
Embodiment 105) The compound of embodiment 100, wherein Z includes four heteroatoms.
Embodiment 106) The compound of embodiment 100, where Z is selected from the group consisting of pyrrole, thiophene, thiazole, isoxazole, and tetrazole.
Embodiment 107) The compound of any one of embodiments 100-106, wherein Ar1 is phenyl.
Embodiment 108) The compound of any one of embodiments 100-106, wherein Ar1 is pyridine.
Embodiment 109) The compound of any one of embodiments 100-106, wherein Ar1 is furan.
Embodiment 110) The compound of any one of embodiments 100-106, wherein Ar1 is carbazole.
Embodiment 111) The compound of any one of embodiments 100-106, wherein Ar1 is naphthyl.
Embodiment 112) The compound of any one of embodiments 100-106, wherein Ar1 is pyridazine.
Embodiment 113) The compound of any one of embodiments 100-106, wherein Ar1 is thiophene.
Embodiment 114) The compound of any one of embodiments 100-106, wherein Ar1 is pyrrole.
Embodiment 115) The compound of any one of embodiments 100-106, wherein Ar1 is dibenzofuran.
Embodiment 116) The compound of any one of embodiments 100-106, wherein Ar1 is naphthyridine.
Embodiment 117) The compound of any one of embodiments 100-116, wherein each substituent of Ar1 is independently selected from —C(O)—CH₃, —C(O)—NH₂, —C(O)O—CH₂CH₃, —CF₃, —C(O)O-t-butyl, —C(O)—N(CH₃)₂, —C(O)—N(H)CH₃, —C(O)—N(H)CH₂CH₃, —C(O)—N(H)(cycloalkyl), —C(O)—N(H)(CH₂)₄, —C(O)—N(CH₂CH₃)₂, —C(O)—N(H)(t-butyl), —C(O)—N(H)(C₆-cycloalkyl), —C(O)—N(H)(CH₂)₃, —C(O)—N(H)(C₅-cycloalkyl), —C(O)—N(H)(C₄-cycloalkyl), —C(O)—N(H)(phenyl), —C(O)—N(H)(CH₂CH₂)N(H)C(O)—CH₃, —C(O)—N(H)C(NH)(NH₂), —C(O)—N(CH₂CH₂CH₃)₂, —C(O)—N—(C(H)(CH₃)(CH₂CH₃))₂, —C(O)—N(CH₂CH₂CH₂CH₃)₂, —C(O)—N(H)(benzyl), —C(O)—N(H)—CH₂CH₂OH, —C(O)—S—CH₂CH₃, —C(O)N(CH₂)₂, —N(H)S(O)(O)-phenyl, —C(O)N(H)CH₂CH₂CF₃, —C(O)N(H)CH₃, —C(O)OCH₂CH₃, —C(O)—N(H)—CH₂CH₂O—CH₂CH₂O—CH₃, —C(O)—N(H)—(CH₂CH₂O)₈—CH₃, —C(O)N(H)—CH₂—CH₂—CH₂—N(H)—CH₂—CH₂—CH₂—N(H)—CH₂—CH₂—CH₂—NH₂, —C(O)N(H)—CH₂—CH₂—NH₂, —C(O)N(H)C(O)CH₃, —C(O)N(H)—CH₂—CH₃, —S(O)(O)N(H)—CH₂—CH₃, —S(O)(O)N—(CH₂—CH₃)₂, —S(O)(O)N(H)—(C₃-cycloalkyl), —S(O)(O)N(H)—(CH₂CH₂)OH, —S(O)(O)OH, —C(O)N(H)C(H)(CH₂OH)₂, —C(O)N(CH₂CH₂OH)₂, —C(O)N(H)CH₂CH₂═CH₂, —C(O)CH₂N(CH₃)(CH₂)(COOH), —S(O)(O)(CH₂CH₃), —S(O)(O)N(H)CH₂—CH(OH)(CH₂OH), —S(O)(O)—NH₂, and —S(O)(O)N(H)CH₂—C(O)(O-t-butyl).
Embodiment 118) The compound of any one of embodiments 100-116, wherein Ar1 is substituted with at least one —SO₂—N(H)(R¹) group.
Embodiment 119) The compound of embodiment 118, wherein R¹ is a C₁-C₆ alkyl.
Embodiment 120) The compound of embodiment 119, wherein the C₁-C₆ alkyl is methyl or ethyl.
Embodiment 121) The compound of any one of embodiments 100-120, wherein Ar2 is selected from the group consisting of benzene, pyridine, pyridazine, pyrimidine and pyrazine.
Embodiment 122) The compound of any one of embodiments 100-121, wherein each Ar2 is substituted with one or more moieties selected from the group consisting of —NH₂, —C(O)OH, —OH, —OCH₃, —C(O)NHOH, —C(O)NH₂, —(CH₂)₃C(O)OH, —CF₃, —Cl, —P(O)(OH)₂, —F, —C(O)N(H)CH₂CH₃, —C(O)N(H)CH₂C(O)OCH₃, —C(O)N(H)CH₂CF₂CF₂CF₃, —C(O)N(H)CH₂C(O)OH, —C(O)N(H)C(H)(C(O)OOH)CH₂C(O)OH, —C(O)N(H)CH₂C(O)N(H)CH₂C(O)OH, —SO₃H, —NO₂, —C(O)N(H)CH₂PO₃H₂, —C(O)N(H)CH₂3O₃H, —B(OH)₂, —C(OH)(CF₃)(CF₃), —C(O)-5-membered heterocycloalkyl-C(O)OH, —C(O)N(H)C(H)(C(O)OH)CH₂-imidazole, —S(O)(O)N(H)—CH₂COOH, —C(O)N(H)CH(C(O)OH)(CH₂CH₂C(O)OH), —C(O)N(H)CH₂CH₂SO₃H, —SO₂O⁻, —C(O)N(H)-imidazole-C(O)OH, and —C(O)N(CH₃)₂.
Embodiment 123) The compound of any one of embodiments 100-121, wherein each Ar2 is substituted with at least one —SO₃H moiety or at least one —SO₃⁻ moiety.
Embodiment 124) The compound of any one of embodiments 100-121, wherein each Ar2 is substituted with at least two —SO₃H moieties or at least two —SO₃⁻ moieties.
Embodiment 125) The compound of any one of embodiments 100-121, wherein each Ar2 is substituted with at least two moieties, wherein a first of the at least two moieties comprises one of a —SO₃H moiety or a —SO₃⁻ moiety.
Embodiment 126) The compound of embodiment 125, wherein a second of the at least two moieties is selected from the group consisting of —NO₂, —CF₃, —F, —Cl, —I, methyl, ethyl, propyl, and butyl.
Embodiment 127) The compound of any one of embodiments 100-121, wherein the compound has any one of Formulas (IA) or (IB):

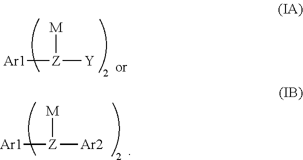

Embodiment 128) The compound of any one of embodiments 100-121, wherein the compound has the formula:

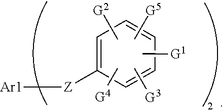

Embodiment 129) The compound of any one of embodiments 100-121, wherein the compound has any one of the formulas:

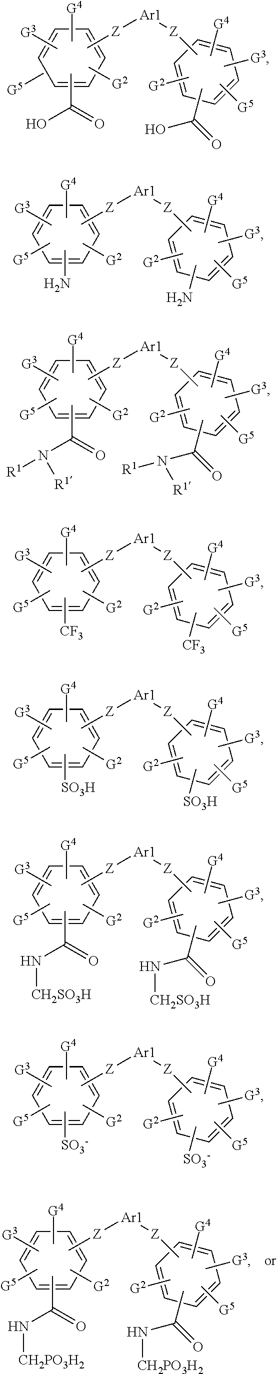

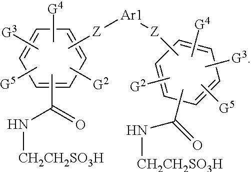

Embodiment 130) The compound of any one of embodiments 100-121, wherein the compound has any one of the formulas:

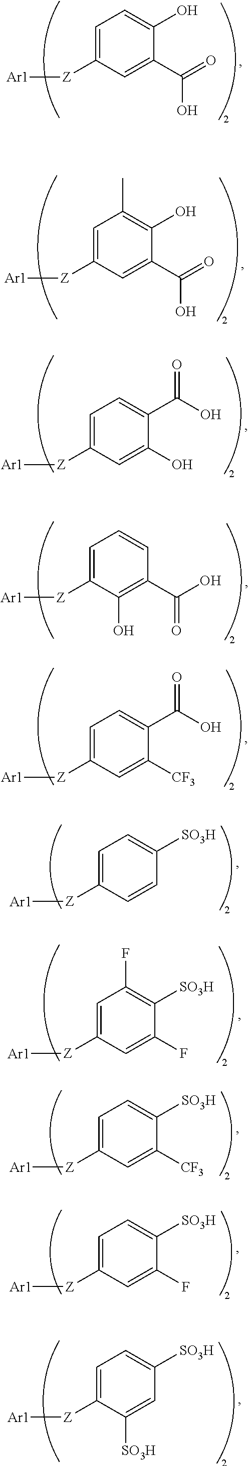

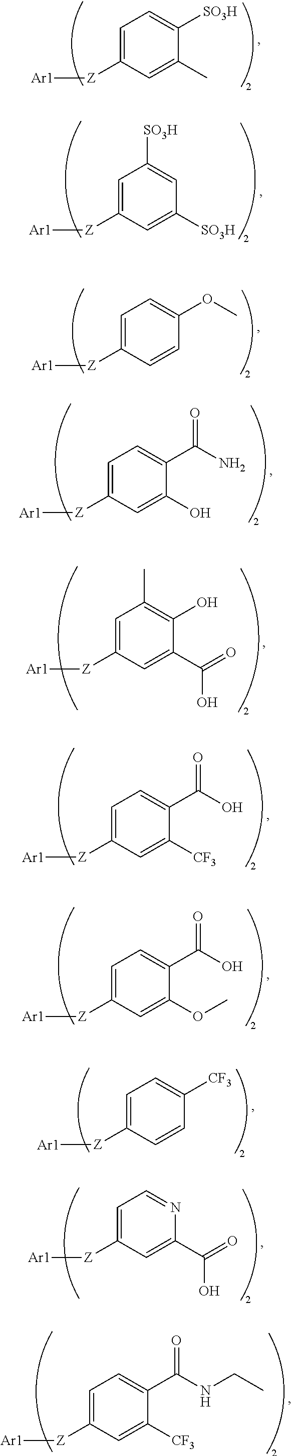

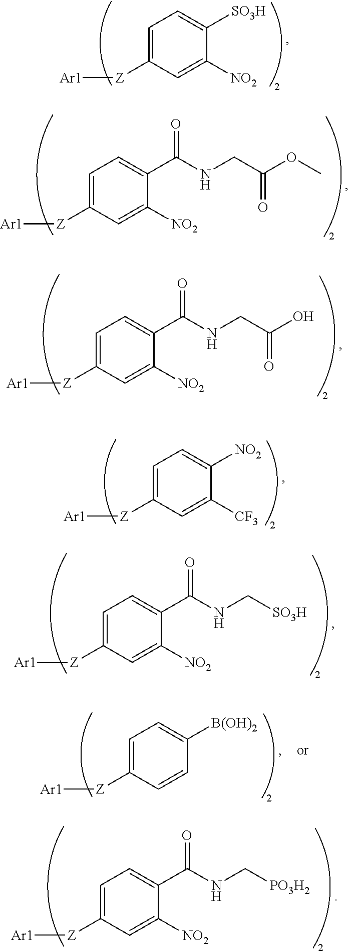

Embodiment 131) A compound having any one of Formulas IA or (IB):

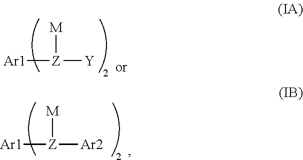

or a solvate, hydrate, tautomer, chelate or salt thereof,
wherein
   a is 0 or an integer ranging from 1-4;
   a′ is 0 or an integer ranging from 1-4;
   m is 1, 2 or 3;
   m′ is 1, 2 or 3;
   n is 0, 1 or 2;
   p is 0, 1 or 2;
   Z is a 5-membered or 6-membered heteroaromatic ring including one, two, three, or four heteroatoms selected from O, N, or S;
   Y is, at each occurrence, independently selected from Ar2, —C(O)—Ar2, —(CH₂)ₐAr2, —(CH₂)₃PO(OEt)₂, or —CH₂CO₂Me;
   M is, at each occurrence, independently selected from hydrogen, halogen and C₁-C₄ alkyl;
      Ar1 is, at each occurrence, independently selected from optionally substituted phenyl, pyridine, bipyridine, tripyridine, pyrazine, pyridazine, furan, dibenzofuran, thiophene, pyrrole, selenophene, naphthalene, fluorene, phenanthrene, cinnoline, phthalazine, quinazoline, quinoxaline, naphthyridine, phenanthroline, purine, and carbazole,
      wherein Ar1 is substituted with at least one of —OH, —C(O)NR¹R^{1′}, —C(O)(CH₂)ₐNR¹R^{1′}, —CO(CH₂)ₐOC(O)R³, —OC(O)R³, —S(O)₂R¹, —S(O)₂NR¹R^{1′}, —CH₂—NR¹S(O)₂R³, or —NR¹S(O)₂R³;
      R⁰ is, at each occurrence, independently selected from C₁-C₆ alkyl, C₁-C₆ haloalkyl, C₂-C₆ alkenyl, C₂-C₆ alkynyl, substituted or unsubstituted cycloalkyl, substituted or unsubstituted heterocyclyl, substituted or unsubstituted aryl, and substituted or unsubstituted heteroaryl;
      R¹ and R^{1′} are, at each occurrence, independently selected from H, hydroxyl, C₁-C₆ alkyl, C₁-C₆ haloalkyl, C₁-C₂₀ heteroalkyl, C₁-C₁₀ heteroalkyl-NH₂, C₂-C₆ alkenyl, C₂-C₆ alkynyl, substituted or unsubstituted cycloalkyl, substituted or unsubstituted heterocyclyl, substituted or unsubstituted aryl, substituted or unsubstituted arylalkyl, and substituted or unsubstituted heteroaryl, benzyl, —C(═NH)NH₂, —CH₂CO₂R⁰, —C(H)(CHₐ(COOH))₂, —CH₂C(O)NHCH₂CO₂H, —CH₂(CH₂)ₐOH, —CH₂CH₂NHC(O)R³, —(CH₂)ₐC(O)NH(CH₂)ₐC(O)OR³, —(CH₂)ₐ—CN, spermine, —(CH₂)ₐNH₂, —C(O)R³, —(CH₂)ₐC(O)OR³, —(CH₂)ₐOR³, —C(H)((CH₂)ₐOH)_{a′}, —CHCH(OH)(CH₂)ₐOH, —(CH₂)ₐOH, C₁-C₆—C(O)OH, —(CH₂)ₐ-heterocycle which may be substituted or unsubstituted

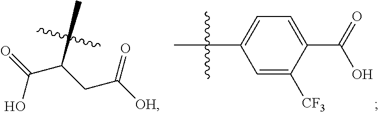

and
wherein R¹ and R^{1′} together form a substituted or unsubstituted heterocyclic ring, including, but not limited to, azetidine, pyrrolidine, piperidine, piperazine, morpholine,

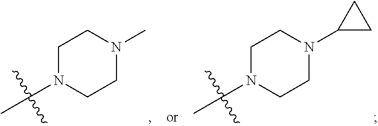

R² is, at each occurrence, independently selected from C₂-C₆ alkyl, C₁-C₆ haloalkyl, C₁-C₆ heteroalkyl, or substituted or unsubstituted cycloalkyl, substituted or unsubstituted heterocyclyl, substituted or unsubstituted aryl, substituted or unsubstituted heteroaryl, and substituted or unsubstituted haloalkoxy;
R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine;
Ar2 is, at each occurrence, independently selected from (i) a substituted or unsubstituted 5- and 6-membered monocyclic aromatic or heteroaromatic ring; (ii) a substituted or unsubstituted 9- and 10-membered fused bicyclic rings comprising two monocyclic rings together, where at least one of the two monocyclic rings is an aromatic or a heteroaromatic ring; and (iii) a substituted or unsubstituted 13- and 14-membered fused tricyclic rings comprising three monocyclic rings together, where at least one of the three monocyclic rings is an aromatic or a heteroaromatic ring;
wherein each Ar2 is independently substituted with G¹, G², G³, G⁴ and G⁵, wherein:
when Ar2 is monosubstituted, G¹ is, at each occurrence, independently selected from oxo, C₁-C₆ alkyl, C₁-C₆ haloalkyl, -E-O—R³, -E-C(R¹)(R^{1′})(R³), —NH₂, —NO₂, —SO₃R³, —SO₃O⁻, —SO₃N(H)(R′), -E-C(O)R³, -E-CO₂H, —B(OH)₂, —C(O)NR¹R^{1′}, -E-PO(OR¹)₂, and aryl substituted with G², G³, G⁴ and G⁵;
G², G³, G⁴ and G⁵ are, at each occurrence, independently selected from absent or selected from the groups comprising, halogen, —CN, —NO₂, —SO₃R³, C₁-C₆ alkyl, C₁-C₆ haloalkyl, -E-O—R³, E-(CH₂)ₐC(O)R³, -E-CO₂H, -E-CHO, -E-C(O)R³, -E-C(O)NH(OH), -E-C(O)NHR¹, -E-C(O)N(H)C(H)(R¹)(R^{1′}), -E-C(O)N(R¹)((CH₂)ₐP(O)(O)OH)₂, -E-C(O)N(R¹)((CH₂)ₐSO₃H), -E-C(O)NR¹R^{1′}, -E-NR¹R^{1′}, -E-OR², —C(O)-heterocycloalkyl (where the heterocycloalkyl is substituted or unsubstituted), —C(O)—N(H)—CH(COOH)((CH₂)ₐ-heteroaryl) (where heteroaryl is substituted or unsubstituted), wherein E is, at each occurrence, independently selected from a direct bond, and C₁-C₆ alkylene.
Embodiment 132) The compound of embodiment 131, wherein at least one Ar1 substituent is selected from the group consisting of —OH, —C(O)NR¹R^{1′}, —C(O)(CH₂)ₐNR¹R^{1′}, —CO(CH₂)ₐOC(O)R³, —OC(O)R³, —S(O)₂R¹, and —S(O)₂NR¹R^{1′}.
Embodiment 133). The compound of claim 131, wherein Ar1 is substituted with at least one —C(O)(CH₂)ₐNR¹R^{1′}.
Embodiment 134) The compound of embodiment 133, wherein R¹ is H.
Embodiment 135) The compound of embodiment 134, wherein R^{1′} is C₁-C₆ alkyl.
Embodiment 136) The compound of embodiment 135, wherein the C₁-C₆ alkyl is branched.
Embodiment 137) The compound of embodiment 135, wherein the C₁-C₆ alkyl comprises at least one substituent.
Embodiment 138) The compound of embodiment 136, wherein the at least one substituent is hydroxyl.
Embodiment 139) The compound of embodiment 133, wherein R¹ and R^{1′} together form a cycloalkyl group or a heterocycloalkyl group.
Embodiment 140) The compound of embodiment 131, wherein Ar1 is substituted with at least one —S(O)₂NR¹R^{1′}.
Embodiment 141) The compound of embodiment 140, wherein R¹ is H.
Embodiment 142) The compound of embodiment 141, wherein R^{1′} is C₁-C₆ alkyl.
Embodiment 143) The compound of embodiment 142, wherein the C₁-C₆ alkyl is branched.
Embodiment 144) The compound of embodiment 142, wherein the C₁-C₆ alkyl comprises at least one substituent.
Embodiment 145) The compound of embodiment 144, wherein the at least one substituent is hydroxyl.
Embodiment 146) The compound of embodiment 140, wherein R¹ and R^{1′} together form a cycloalkyl group or a heterocycloalkyl group.
Embodiment 147) The compound of any one of embodiments 131-146, wherein each Ar2 is substituted with at least one —SO₃H moiety or at least one —SO₃⁻ moiety.
Embodiment 148) The compound of any one of embodiments 131-146, wherein each Ar2 is substituted with at least two —SO₃H moieties or at least two —SO₃⁻ moieties.
Embodiment 149) The compound of any one of embodiments 131-146, wherein each Ar2 is substituted with at least two moieties, wherein a first of the at least two moieties comprises one of a —SO₃H moiety or a —SO₃⁻ moiety.
Embodiment 150) The compound of embodiment 149, wherein a second of the at least two moieties is selected from the group consisting of —NO₂, —CF₃, —F, —Cl, —I, methyl, ethyl, propyl, and butyl.
Embodiment 151) The compound of any one of embodiments 131-145, wherein the compound has any one of the formulas:

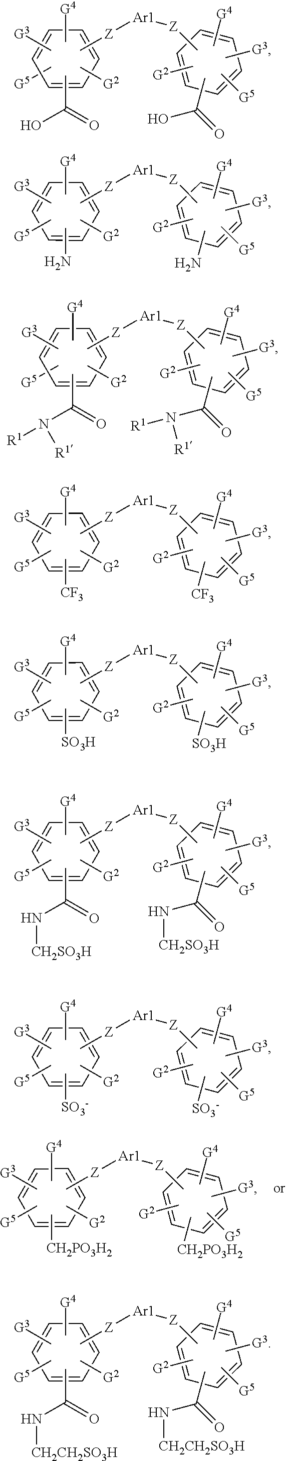

Embodiment 152) The compound of any one of embodiments 131-145, wherein the compound has any one of the formulas.

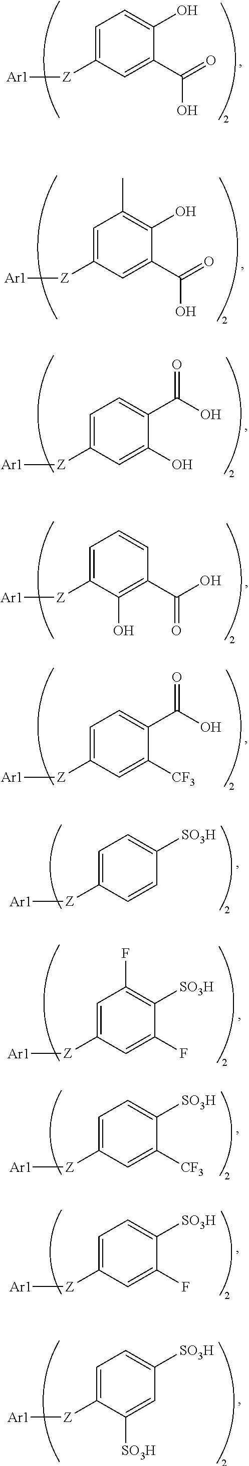

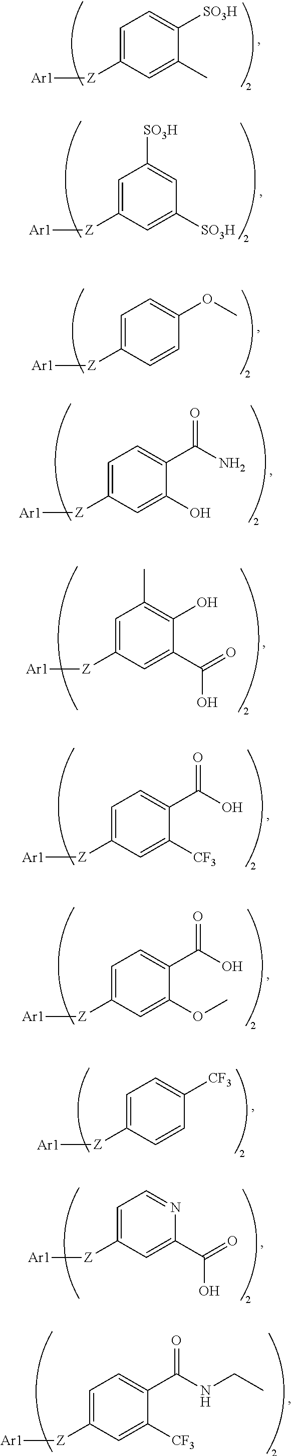

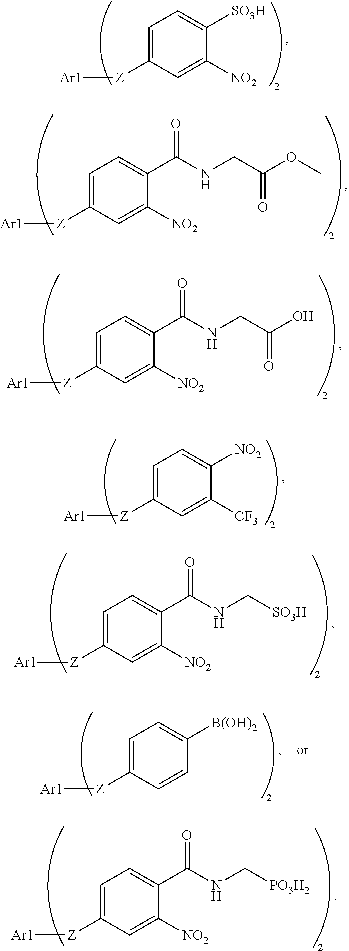

Embodiment 153) The compound of embodiment 131, wherein the compound has the formula:

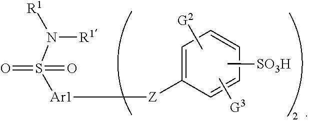

Embodiment 154) The compound of embodiment 131, wherein the compound has the formula:

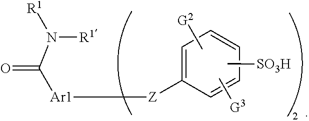

Embodiment 155) The compound of any one of embodiments 131-154, wherein Z is a diazole.
Embodiment 156) The compound of any one of embodiments 131-154, wherein Z is selected from the group consisting of imidazole, pyrazole, pyrimidine, pyridazine, and pyrazine.
Embodiment 157) The compound of any one of embodiments 131-154, wherein Z is not a triazole.
Embodiment 158) The compound of any one of embodiments 131-146, wherein the compound has any one of the formulas:

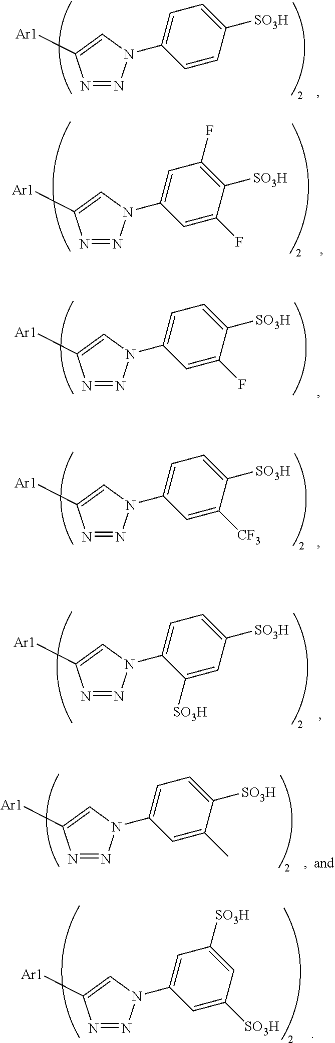

Embodiment 159) A compound having any one of the formulas:

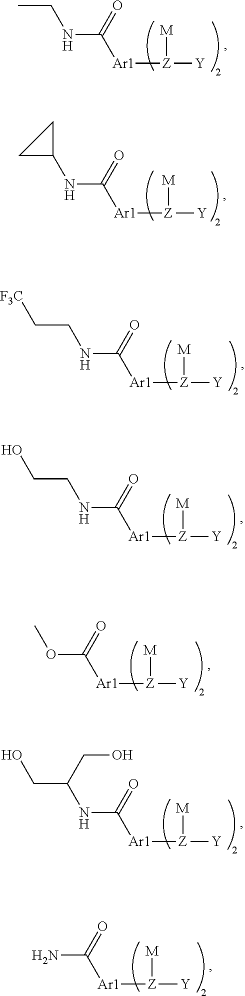

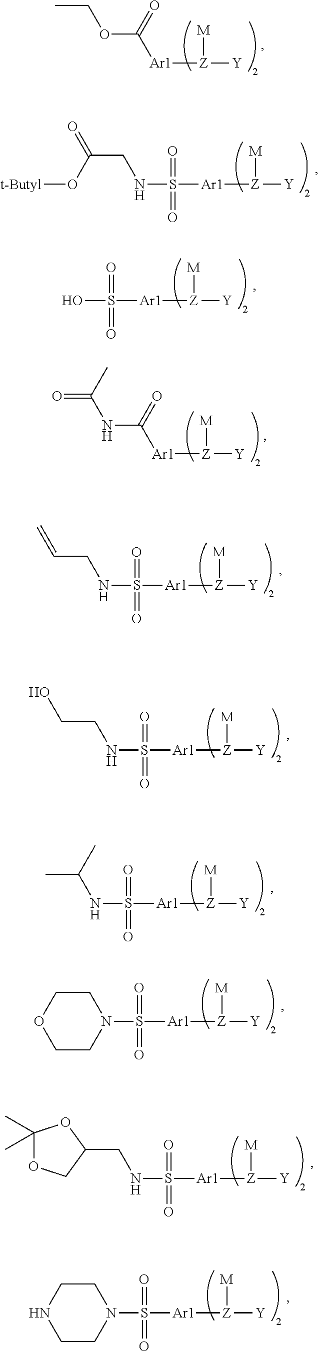

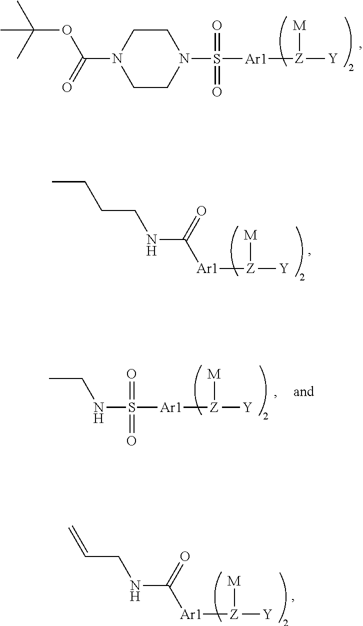

or a solvate, hydrate, tautomer, chelate or salt thereof,
wherein
   Z is a 5-membered or 6-membered heteroaromatic ring including one, two, three, or four heteroatoms selected from O, N, or S;
   M is, at each occurrence, independently selected from hydrogen, halogen and C₁-C₄ alkyl;
   Y is, at each occurrence, independently selected from Ar2, —C(O)—Ar2, —(CH₂)ₐAr2, —(CH₂)₃PO(OEt)₂, or —CH₂CO₂Me;
   Ar1 is, at each occurrence, independently selected from optionally substituted phenyl, pyridine, bipyridine, tripyridine, pyrazine, pyridazine, furan, dibenzofuran, thiophene, pyrrole, selenophene, naphthalene, fluorene, phenanthrene, cinnoline, phthalazine, quinazoline, quinoxaline, naphthyridine, phenanthroline, purine, and carbazole;
   Ar2 is, at each occurrence, independently selected from (i) a substituted or unsubstituted 5- and 6-membered monocyclic aromatic or heteroaromatic ring; (ii) a substituted or unsubstituted 9- and 10-membered fused bicyclic rings comprising two monocyclic rings together, where at least one of the two monocyclic rings is an aromatic or a heteroaromatic ring; and (iii) a substituted or unsubstituted 13- and 14-membered fused tricyclic rings comprising three monocyclic rings together, where at least one of the three monocyclic rings is an aromatic or a heteroaromatic ring;
   wherein each Ar2 is independently substituted with G¹, G², G³, G⁴ and G⁵, wherein:
   when Ar2 is monosubstituted, G¹ is, at each occurrence, independently selected from oxo, C₁-C₆ alkyl, C₁-C₆ haloalkyl, -E-O—R³, -E-C(R¹)(R′)(R³), —NH₂, —NO₂, —SO₃R³, —SO₃O⁻, —SO₃N(H)(R′), -E-C(O)R³, -E-CO₂H, —B(OH)₂, —C(O)NR¹R^{1′}, -E-PO(OR¹)₂, and aryl substituted with G², G³, G⁴ and G⁵;
   G², G³, G⁴ and G⁵ are, at each occurrence, independently selected from absent or selected from the groups comprising, halogen, —CN, —NO₂, —SO₃R³, C₁-C₆ alkyl, C₁-C₆ haloalkyl, -E-O—R³, E-(CH₂)ₐC(O)R³, -E-CO₂H, -E-CHO, -E-C(O)R³, -E-C(O)NH(OH), -E-C(O)NHR¹, -E-C(O)N(H)C(H)(R¹)(R^{1′}), -E-C(O)N(R¹)((CH₂)ₐP(O)(O)OH)₂, -E-C(O)N(R¹)((CH₂)ₐSO₃H), -E-C(O)NR¹R^{1′}, -E-NR¹R^{1′}, -E-OR², —C(O)-heterocycloalkyl (where the heterocycloalkyl is substituted or unsubstituted), —C(O)—N(H)—CH(COOH)((CH₂)ₐ-heteroaryl) (where heteroaryl is substituted or unsubstituted), wherein E is, at each occurrence, independently selected from a direct bond, and C₁-C₆ alkylene;
   R⁰ is, at each occurrence, independently selected from C₁-C₆ alkyl, C₁-C₆ haloalkyl, C₂-C₆ alkenyl, C₂-C₆ alkynyl, substituted or unsubstituted cycloalkyl, substituted or unsubstituted heterocyclyl, substituted or unsubstituted aryl, and substituted or unsubstituted heteroaryl;
   R¹ and R^{1′} are, at each occurrence, independently selected from H, hydroxyl, C₁-C₆ alkyl, C₁-C₆ haloalkyl, C₁-C₂₀ heteroalkyl, C₁-C₁₀ heteroalkyl-NH₂, C₂-C₆ alkenyl, C₂-C₆ alkynyl, substituted or unsubstituted cycloalkyl, substituted or unsubstituted heterocyclyl, substituted or unsubstituted aryl, substituted or unsubstituted arylalkyl, and substituted or unsubstituted heteroaryl, benzyl, —C(═NH)NH₂, —CH₂CO₂R⁰, —C(H)(CHₐ(COOH))₂, —CH₂C(O)NHCH₂CO₂H, —CH₂(CH₂)ₐOH, —CH₂CH₂NHC(O)R³, —(CH₂)ₐC(O)NH(CH₂)ₐC(O)OR³, —(CH₂)ₐ—CN, spermine, —(CH₂)ₐ—NH₂, —C(O)R³, —(CH₂)ₐC(O)OR³, —(CH₂)ₐOR³, —C(H)((CH₂)ₐOH)_{a′}, —CHCH(OH)(CH₂)ₐOH, —(CH₂)ₐOH, C₁-C₆—C(O)OH, —(CH₂)ₐ-heterocycle which may be substituted or unsubstituted,

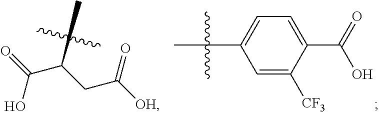

and
wherein R¹ and R^{1′} together form a substituted or unsubstituted heterocyclic ring, including, but not limited to, azetidine, pyrrolidine, piperidine, piperazine, morpholine,

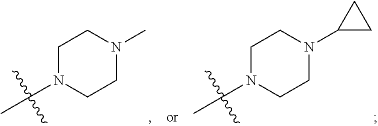

R² is, at each occurrence, independently selected from C₂-C₆ alkyl, C₁-C₆ haloalkyl, C₁-C₆ heteroalkyl, or substituted or unsubstituted cycloalkyl, substituted or unsubstituted heterocyclyl, substituted or unsubstituted aryl, substituted or unsubstituted heteroaryl, and substituted or unsubstituted haloalkoxy; and
R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine.
Embodiment 160) The compound of embodiment 159, wherein Z includes two heteroatoms.
Embodiment 161) The compound of embodiment 160, wherein Z is a diazole.
Embodiment 162) The compound of embodiment 161, wherein the diazole is selected from the group consisting of imidazole, pyrazole, pyrimidine, pyridazine, and pyrazine.
Embodiment 163) The compound of embodiment 159, wherein Z includes three heteroatoms.
Embodiment 164) The compound of embodiment 159, wherein Z includes four heteroatoms.
Embodiment 165) The compound of embodiment 159, where Z is selected from the group consisting of pyrrole, thiophene, thiazole, isoxazole, and tetrazole.
Embodiment 166) The compound of embodiment 159, wherein Z is a triazole.
Embodiment 167) The compound of embodiment 159, wherein Z is not a triazole.
Embodiment 168) The compound of any one of embodiments 159-167, wherein Ar1 is phenyl.
Embodiment 169) The compound of any one of embodiments 159-167, wherein Ar1 is pyridine.
Embodiment 170) The compound of any one of embodiments 159-167, wherein Ar1 is furan.
Embodiment 171) The compound of any one of embodiments 159-167, wherein Ar1 is carbazole.
Embodiment 172) The compound of any one of embodiments 159-167, wherein Ar1 is naphthyl.
Embodiment 173) The compound of any one of embodiments 159-167, wherein Ar1 is pyridazine.
Embodiment 174) The compound of any one of embodiments 159-167, wherein Ar1 is thiophene.
Embodiment 175) The compound of any one of embodiments 159-167, wherein Ar1 is pyrrole.
Embodiment 176) The compound of any one of embodiments 159-167, wherein Ar1 is dibenzofuran.
Embodiment 177) The compound of any one of embodiments 159-167, wherein Ar1 is naphthyridine.
Embodiment 178) The compound of any one of embodiments 159-167, wherein each Ar2 is substituted with one or more moieties selected from the group consisting of —NH₂, —C(O)OH, —OH, —OCH₃, —C(O)NHOH, —C(O)NH₂, —(CH₂)₃C(O)OH, —CF₃, —Cl, —P(O)(OH)₂, —F, —C(O)N(H)CH₂CH₃, —C(O)N(H)CH₂C(O)OCH₃, —C(O)N(H)CH₂CF₂CF₂CF₃, —C(O)N(H)CH₂C(O)OH, —C(O)N(H)C(H)(C(O)OOH)CH₂C(O)OH, —C(O)N(H)CH₂C(O)N(H)CH₂C(O)OH, —SO₃H, —NO₂, —C(O)N(H)CH₂PO₃H₂, —C(O)N(H)CH₂3O₃H, —B(OH)₂, —C(OH)(CF₃)(CF₃), —C(O)-5-membered heterocycloalkyl-C(O)OH, —C(O)N(H)C(H)(C(O)OH)CH₂-imidazole, —S(O)(O)N(H)—CH₂COOH, —C(O)N(H)CH(C(O)OH)(CH₂CH₂C(O)OH), —C(O)N(H)CH₂CH₂SO₃H, —SO₂O⁻, —C(O)N(H)-imidazole-C(O)OH, and —C(O)N(CH₃)₂.
Embodiment 179) The compound of any one of embodiments 159-177, wherein each Ar2 is substituted with at least one —SO₃H moiety or at least one —SO₃⁻ moiety.
Embodiment 180) The compound of any one of embodiments 159-177, wherein each Ar2 is substituted with at least two —SO₃H moieties or at least two —SO₃⁻ moieties.
Embodiment 181) The compound of any one of embodiments 159-177, wherein each Ar2 is substituted with at least two moieties, wherein a first of the at least two moieties comprises one of a —SO₃H moiety or a —SO₃⁻ moiety.
Embodiment 182) The compound of embodiment 181, wherein a second of the at least two moieties is selected from the group consisting of —NO₂, —CF₃, —F, —Cl, —I, methyl, ethyl, propyl, and butyl.
Embodiment 183) A compound having any one of the formulas:

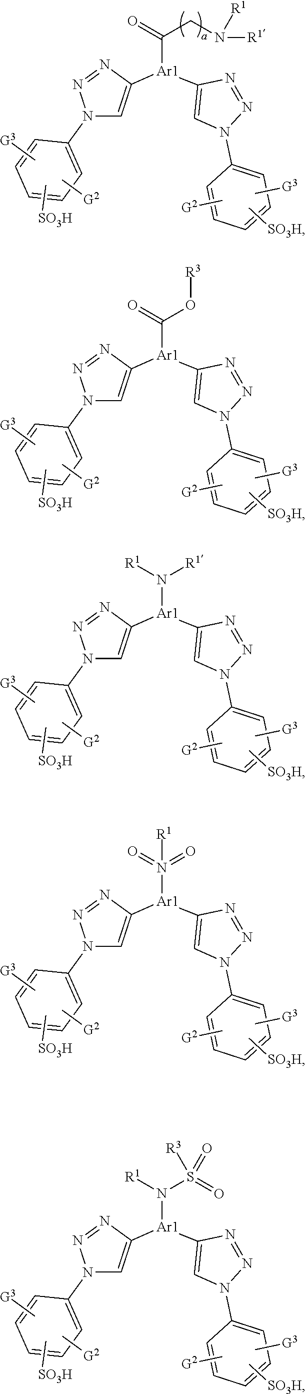

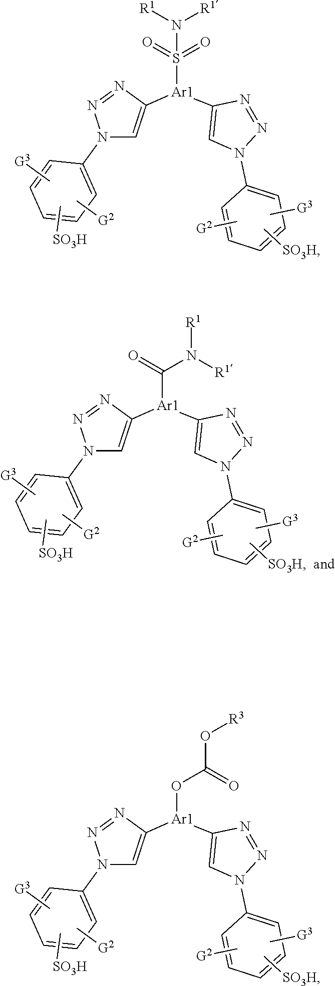

or a solvate, hydrate, tautomer, chelate or salt thereof,
wherein
Ar1 is, at each occurrence, independently selected from optionally substituted phenyl, pyridine, bipyridine, tripyridine, pyrazine, pyridazine, furan, dibenzofuran, thiophene, pyrrole, selenophene, naphthalene, fluorene, phenanthrene, cinnoline, phthalazine, quinazoline, quinoxaline, naphthyridine, phenanthroline, purine, and carbazole;
R¹ and R^{1′} are, at each occurrence, independently selected from H, hydroxyl, C₁-C₆ alkyl, C₁-C₆ haloalkyl, C₁-C₂o heteroalkyl, C₁-C₁₀ heteroalkyl-NH₂, C₂-C₆ alkenyl, C₂-C₆ alkynyl, substituted or unsubstituted cycloalkyl, substituted or unsubstituted heterocyclyl, substituted or unsubstituted aryl, substituted or unsubstituted arylalkyl, and substituted or unsubstituted heteroaryl, benzyl, —C(═NH)NH₂, —CH₂CO₂R⁰, —C(H)(CHₐ(COOH))₂, —CH₂C(O)NHCH₂CO₂H, —CH₂(CH₂)ₐOH, —CH₂CH₂NHC(O)R³, —(CH₂)ₐC(O)NH(CH₂)ₐC(O)OR³, —(CH₂)ₐ—CN, spermine, —(CH₂)ₐ—NH₂, —C(O)R³, —(CH₂)ₐC(O)OR³, —(CH₂)ₐOR³, —C(H)((CH₂)ₐOH)_{a′}, —CHCH(OH)(CH₂)ₐOH, —(CH₂)ₐOH, C₁-C₆—C(O)OH, —(CH₂)ₐ-heterocycle which may be substituted or unsubstituted,

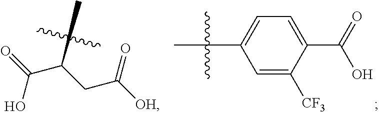

and
wherein R¹ and R^{1′} together form a substituted or unsubstituted heterocyclic ring, including, but not limited to, azetidine, pyrrolidine, piperidine, piperazine, morpholine,

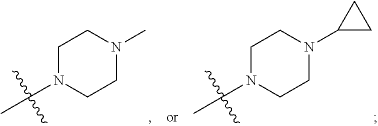

R² is, at each occurrence, independently selected from C₂-C₆ alkyl, C₁-C₆ haloalkyl, C₁-C₆ heteroalkyl, or substituted or unsubstituted cycloalkyl, substituted or unsubstituted heterocyclyl, substituted or unsubstituted aryl, substituted or unsubstituted heteroaryl, and substituted or unsubstituted haloalkoxy;
R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine; and
G² and G³ are, at each occurrence, independently selected from absent or selected from the groups comprising, halogen, —CN, —NO₂, —SO₃R³, C₁-C₆alkyl, C₁-C₆ haloalkyl, -E-O—R³, E-(CH₂)ₐC(O)R³, -E-CO₂H, -E-CHO, -E-C(O)R³, -E-C(O)NH(OH), -E-C(O)NHR¹, -E-C(O)N(H)C(H)(R¹)(R^{1′}), -E-C(O)N(R¹)((CH₂)ₐP(O)(O)OH)₂, -E-C(O)N(R¹)((CH₂)ₐSO₃H), -E-C(O)NR¹R^{1′}, -E-NR¹R^{1′}, -E-OR², —C(O)-heterocycloalkyl (where the heterocycloalkyl is substituted or unsubstituted), —C(O)—N(H)—CH(COOH)((CH₂)ₐ-heteroaryl) (where heteroaryl is substituted or unsubstituted), wherein E is, at each occurrence, independently selected from a direct bond, and C₁-C₆ alkylene.
Embodiment 184) The compound of embodiment 183, wherein the compound has any one of the formulas:

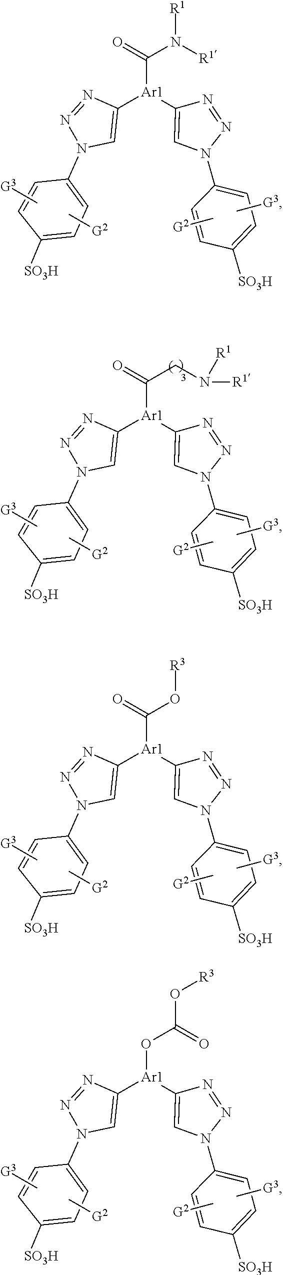

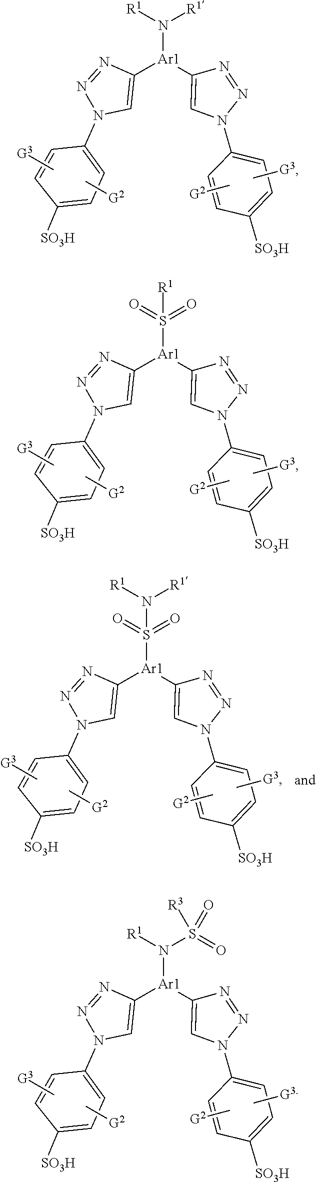

Embodiment 185) The compound of embodiment 183, wherein the compound has any one of the formulas:

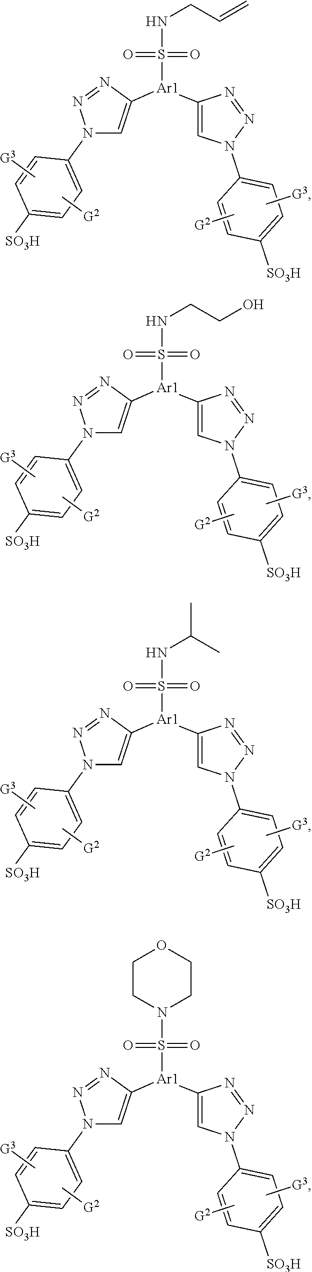

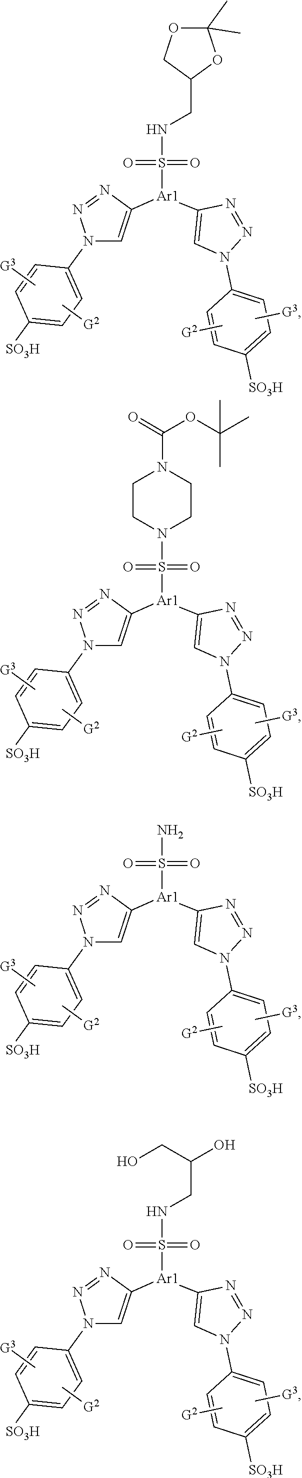

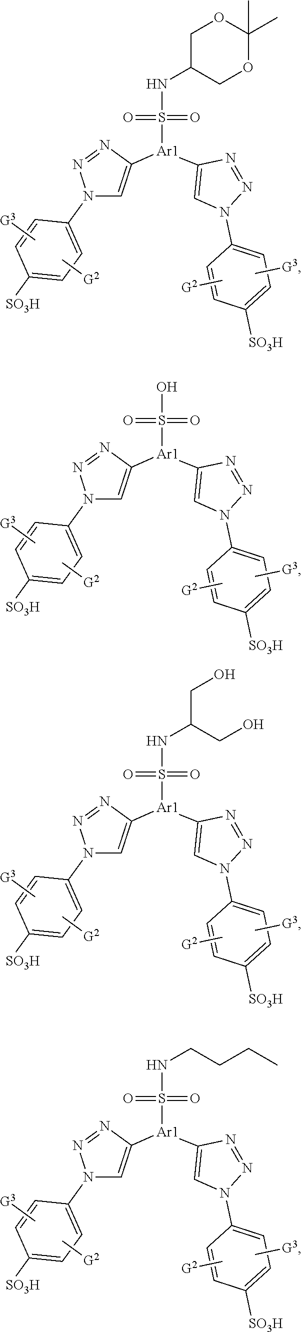

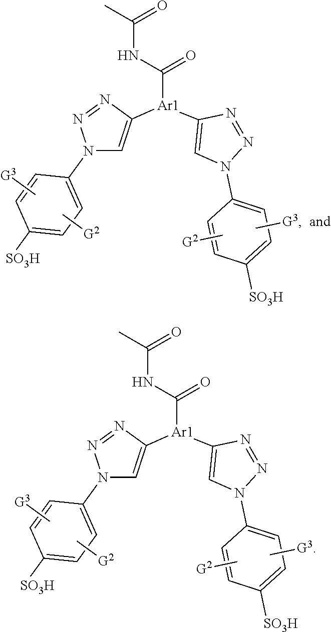

Embodiment 186) The compound of any one of embodiments 183-184, wherein R¹ is H.
Embodiment 187) The compound of embodiment 186, wherein R^{1′} is C₁-C₆ alkyl.
Embodiment 188) The compound of embodiment 187, wherein the C₁-C₆ alkyl is branched.
Embodiment 189) The compound of embodiment 187, wherein the C₁-C₆ alkyl comprises at least one substituent.
Embodiment 190) The compound of embodiment 189, wherein the at least one substituent is hydroxyl.
Embodiment 191) The compound of any one of embodiments 183-184, wherein R¹ and R^{1′} together form a cycloalkyl group or a heterocycloalkyl group.
Embodiment 192) The compound of any one of embodiments 185-191, wherein G² and G³ are each H.
Embodiment 193) The compound of any one of embodiments 183-191, wherein one of G² or G³ is —SO₃H or —SO₃⁻.
Embodiment 194) A compound having the formula:

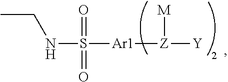

or a solvate, hydrate, tautomer, chelate or salt thereof,
wherein
   Z is a 5-membered or 6-membered heteroaromatic ring including one, two, three, or four heteroatoms selected from O, N, or S;
   M is, at each occurrence, independently selected from hydrogen, halogen and C₁-C₄ alkyl;
   Y is, at each occurrence, independently selected from Ar2, —C(O)—Ar2, —(CH₂)ₐAr2, —(CH₂)₃PO(OEt)₂, or —CH₂CO₂Me;
   Ar1 is, at each occurrence, independently selected from optionally substituted phenyl, pyridine, bipyridine, tripyridine, pyrazine, pyridazine, furan, dibenzofuran, thiophene, pyrrole, selenophene, naphthalene, fluorene, phenanthrene, cinnoline, phthalazine, quinazoline, quinoxaline, naphthyridine, phenanthroline, purine, and carbazole;
   Ar2 is, at each occurrence, independently selected from (i) a substituted or unsubstituted 5- and 6-membered monocyclic aromatic or heteroaromatic ring; (ii) a substituted or unsubstituted 9- and 10-membered fused bicyclic rings comprising two monocyclic rings together, where at least one of the two monocyclic rings is an aromatic or a heteroaromatic ring; and (iii) a substituted or unsubstituted 13- and 14-membered fused tricyclic rings comprising three monocyclic rings together, where at least one of the three monocyclic rings is an aromatic or a heteroaromatic ring;
   wherein each Ar2 is independently substituted with —C(O)OR³, —C₁-C₆ haloalkyl, —NO₂, and —SO₃H;
   R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine.
Embodiment 195) The compound of embodiment 194, wherein the —C₁-C₆ haloalkyl is —CF₃.
Embodiment 196) The compound of embodiment 194, wherein the —C(O)OR³ is —C(O)OH.
Embodiment 197) The compound of embodiment 194, wherein Ar2 comprises two substituents.
Embodiment 198) The compound of embodiment 194, wherein Ar2 comprises three substituents.
Embodiment 199) The compound of embodiment 194, wherein Ar2 comprises four substituents.
Embodiment 200) The compound of embodiment 194, wherein the compound includes one —C(O)OR³ moiety and one —C₁-C₆ haloalkyl moiety.
Embodiment 201) The compound of embodiment 194, wherein the compound includes one —NO₂ moiety and one —SO₃H moiety.
Embodiment 202) The compound of embodiment 194, wherein the compound includes —SO₃H moiety and one —C₁-C₆ haloalkyl moiety.
Embodiment 203) The compound of embodiment 194, wherein the compound includes one —C(O)OR³ moiety and one —NO₂ moiety.
Embodiment 204) The compound of embodiment 194, wherein the compound includes at least one —SO₃H moiety.
Embodiment 205) A polymer, copolymer, or metallogel comprising any one of the compounds of embodiments 100-204.
Embodiment 206) A composition comprising at least two molecules of any one of the compounds of embodiments 100-204.
Embodiment 207) A method of enhancing a nucleic acid polymerase reaction, the method comprising:
   a. forming a nucleic acid polymerase reaction composition comprising:
      i. a template nucleic acid,
      ii. a nucleic acid polymerase,
      iii. a mixture of nucleotides or nucleotide analogs, and
      iv. at least one of the compounds of any one of embodiments 1-105; and
   b. incubating the nucleic acid polymerase reaction composition under conditions allowing a nucleic acid polymerization reaction, wherein the at least one compound of any one of embodiments 100-204 increases the processivity, rate, or fidelity of the nucleic acid polymerase reaction.
Embodiment 208) The method of embodiment 207, wherein the nucleic acid polymerase is a DNA polymerase.
Embodiment 209) The method of embodiment 208, wherein the DNA polymerase is DP04 or a variant thereof.
Embodiment 210) The method of embodiment 207, wherein the mixture of nucleotides or nucleotide analogs is a mixture of nucleotide analogs comprising nucleoside triphosphoramidates, wherein each of the nucleoside triphosphoramidates comprises a nucleobase selected from the group consisting of adenine, guanine, thymine, and cytosine and a polymeric tether moiety, wherein a first end of the polymeric tether moiety is attached to the nucleobase and a second end of the polymeric tether moiety is attached to the alpha phosphate of the nucleoside triphosphoramidate to provide for expansion of the nucleotide analogs by cleavage of the phosphoramidate bond.
Embodiment 211) The method of embodiment 207, wherein the nucleic acid polymerization reaction produces an expandable polymer of nucleotide analogs, wherein the expandable polymer encodes the nucleobase sequence information of the template nucleic acid.
Embodiment 212) The method of embodiment 207, wherein the conditions for allowing a nucleic acid polymerization reaction comprise a suitable polymerization buffer and an oligonucleotide primer.
Embodiment 213) The method of embodiment 212, wherein the suitable polymerization buffer comprises one or more components selected from MnCl₂, a buffer, a salt, a sugar, a single-strand binding protein (SSB), imidazole, pyrazole, triazole, betaine, a molecular crowding agent, dimethyl sulfoxide (DMSO), an alkanediol, glycerol, N-methyl-2-pyrrolidone (NMP), acetamide, butylated hydroxyanisole (BHA), a polyphosphate, and/or urea.
Embodiment 214) The method of embodiment 207, wherein the nucleic acid polymerase reaction composition further comprises a single-strand binding protein.
Embodiment 215) The method of embodiment 207, wherein the nucleic acid polymerase reaction composition further comprises urea.
Embodiment 216) The method of embodiment 207, wherein the mixture of nucleotides or nucleotide analogs comprises nucleotide analogs comprising a detectable label.
Embodiment 217) The method of embodiment 216, wherein the detectable label is an optically detectable label selected from the group consisting of luminescent, chemiluminescent, fluorescent, fluorogenic, chromophoric or chromogenic labels.
Embodiment 218) Use of the compounds of any one of embodiments 100-204 in a nucleic acid polymerase reaction.
Embodiment 219) A method of sequencing a DNA or RNA template, the method comprising the steps of:
   a. forming a DNA polymerase reaction composition comprising:
      i. a DNA or RNA template,
      ii. a replication primer that complexes with the template,
      iii. a DNA polymerase,
      iv. a mixture of nucleotides or nucleotide analogs,
      v. at least one of the compounds of any one of embodiments 100-204;
   b. incubating the DNA polymerase reaction composition under conditions allowing a DNA polymerization reaction, wherein the at least one compound of any one of embodiments 100-204 increases the rate, fidelity or processivity of the DNA polymerase reaction; and
   c. determining the sequence of the nucleotides or nucleotide analogs in the resulting polymer of nucleotides or nucleotide analogs.
Embodiment 220) The method of embodiment 219, wherein the mixture of nucleotide analogs comprises nucleoside triphosphoramidates, wherein each of the nucleoside triphosphoramidates comprises a nucleobase selected from the group consisting of adenine, guanine, thymine, and cytosine and a polymeric tether moiety, wherein a first end of the polymeric tether moiety is attached to the nucleobase and a second end of the polymeric ether moiety is attached to the alpha phosphate of the nucleoside triphosphoramidate to provide for expansion of the nucleotide analogs by cleavage of the phosphoramidate bond.
Embodiment 221) The method of embodiment 220, wherein the DNA polymerase is DP04 or a variant thereof.
Embodiment 222) The method of embodiment 219, wherein the resulting polymer of nucleotide analogs is an expandable polymer.
Embodiment 223) The method of any one of embodiments 219-222, further comprising the step of contacting the expandable polymer with a phosphoramidate cleavage agent to produce an expanded polymer of nucleotide analogs.
Embodiment 224) The method of embodiment 220, wherein the polymeric tether moiety of each of the nucleotide analogs comprises a reporter moiety unique to the nucleobase of the analog.
Embodiment 225) The method of embodiment 224, wherein each of the reporter moieties produce a characteristic electronic signal.
Embodiment 226) The method of embodiment 219, wherein the step of determining the sequence of the nucleotide analogs comprises the step of translocating the expanded polymer of nucleotide analogs through a nanopore.
Embodiment 227) Use of the compounds of any one of embodiments 100-204 in sequencing a DNA or RNA template.
Embodiment 228) A composition comprising any one of the compounds of embodiments 1-105, and a molecular crowding agent.
Embodiment 229) The composition of embodiment 228, wherein the molecular crowding agent is a polyalkylene glycol.
Embodiment 230) A composition comprising any one of the compounds of embodiments 100-204, and a buffer.
Embodiment 231) The composition of embodiment 230, wherein the buffer comprises one or more component selected from MnCl₂, a buffer, a salt, a sugar, a single-strand binding protein (SSB), imidazole, pyrazole, triazole, betaine, a molecular crowding agent, dimethyl sulfoxide (DMSO), an alkanediol, glycerol, N-methyl-2-pyrrolidone (NMP), acetamide, butylated hydroxyanisole (BHA), a polyphosphate, and/or urea.
Embodiment 232) The composition of embodiment 231, wherein the salt is selected from the group consisting of NaCl, NaBr, NaOAc, NaF, sodium formate, sodium phosphate monobasic, sodium phosphate dibasic, NaSO₄, sodium carbonate, sodium bicarbonate, sodium hexanoate, sodium glutamate, sodium perchlorate, CsCl, LiCl, LiOAc, LiF, lithium carbonate, LiPO₄, KCl, KOAc, KF, KSO₄, potassium phosphate monobasic, potassium phosphate dibasic, potassium carbonate, potassium bicarbonate, potassium glutamate, NH₄Cl, NH₄F, NH₄OAc, NH₄SO₄, NH₄Br, ammonium citrate, ammonium carbonate, ammonium bicarbonate, ammonium sulfite, ammonium glutamate, ammonium phosphate monobasic, tetramethylammonium chloride (TMACl), trimethylamine N-oxide (TMAO), tetraethylammonium chloride (TEACl), guanidinium chloride, guanidinium thiocyanate, and guanidium carbonate.
Embodiment 233) The composition of embodiment 231, wherein the salt is an inorganic salt.
Embodiment 234) The composition of embodiment 231, wherein the salt is NaCl or KCl.

The composition of embodiment 132, wherein the sugar is maltose, trehalose, cellobiose or sucrose.
Embodiment 235) The composition of embodiment 231, wherein the imidazole is a derivative or analog of imidazole selected from the consisting of imidazole chloride, imidazole acetate, 1-methylimidazole, 2-methylimidazole, 1-ethylimidazole, 1-ethyl-3-methylimidazolium chloride, 2-methyl-2-imidazoline, 1-butyl-3-methylimidazolium chloride, 1-methylimidazolium chloride, 1-hexyl-3-methylimidazolium, 3-octyl-1-methylimidazolium, and 1-decyl-3-methylimidazolium.
Embodiment 236) The composition of embodiment 231, wherein the alkanediol is ethylenglycol, a propanediol or a butanediol, such as a propanediol.
Embodiment 237) The composition of embodiment 231, wherein the propanediol is 1,2-propanediol or 1,3-propanediol, such as 1,2 propanediol. In some embodiments, the butanediol is 1,2-butanediol, 1,3-butanediol, 1,4-butanediol, 2,3-butanediol, 2,4-butanediol, or 3,4-butanediol, such as 1,2-butanediol.
Embodiment 238) The composition of embodiment 231, wherein the molecular crowding agent is PEG, such as PEG4k to PEG25k or PEG4k to PEG 10k, such as PEG5k, PEG8k, or PEG10k.
Embodiment 239) The composition of embodiment 231, wherein the polyphosphate is tripolyphosphate, tetrapolyphosphate, pentapolyphosphate, hexapolyphosphate, trimetaphosphate (TMP), hexametaphosphate (HMP), or polyphosphate 60.
Embodiment 240) A composition comprising any one of the compounds of embodiments 100-204, and a polynucleotide.
Embodiment 241) The composition of embodiment 240, wherein the polynucleotide is a 20-60 mer oligonucleotide.
Embodiment 242) A composition comprising any one of the compounds of embodiments 100-204, and a protein.
Embodiment 243) The composition of embodiment 242, wherein the protein is a DNA polymerase.
Embodiment 244) A composition comprising any one of the compounds of embodiments 100-204, and a mixture of nucleotides or nucleotide analogs.
Embodiment 245) A composition for enhancing the processivity, fidelity, or rate of a DNA polymerase reaction comprising at least one of the compounds of any one of embodiments 100-204, and a mixture of nucleotide analogs.
Embodiment 246) A composition comprising at least one of the compounds of any one of embodiments 100-204, and a mixture of nucleotide analogs wherein the at least one compound of any of embodiments 100-204, increases the number and accuracy of nucleotide analogs incorporated into a daughter strand during a template-dependent polymerization reaction relative to an identical polymerization reaction absent the at least one compound of any of embodiments 100-204.
Embodiment 247) The composition of embodiment 246, wherein the mixture of nucleotide analogs comprises nucleoside triphosphoramidates, wherein each of the nucleoside triphosphoramidates comprises a nucleobase selected from the group consisting of adenine, guanine, thymine, and cytosine and a polymeric tether moiety, wherein a first end of the polymeric tether moiety is attached to the nucleobase and a second end of the polymeric ether moiety is attached to the alpha phosphate of the nucleoside triphosphoramidate to provide for expansion of the nucleotide analogs by cleavage of the phosphoramidate bond.
Embodiment 248) The composition of embodiment 247, further comprising a buffer component selected from at least one of Tris OAc, NH4OAc, PEG, a water-miscible organic solvent, polyphosphate 60, NMS, and MnCl2.
Embodiment 249) The composition of embodiment 247, further comprising a single-strand binding protein.
Embodiment 250) The composition of embodiment 247, further comprising urea.
Embodiment 251) The composition of embodiment 247, wherein the mixture of nucleotide analogs comprises nucleotide analogs comprising a detectable label.
Embodiment 252) The composition of embodiment 251, wherein the detectable label is an optically detectable label selected from the group consisting of luminescent, chemiluminescent, fluorescent, fluorogenic, chromophoric or chromogenic labels.

The above-mentioned and additional features of the present disclosure as well as the manner of obtaining them will become apparent, and the present disclosure will be best understood by reference to the following more detailed description. All references disclosed herein are hereby incorporated by reference in their entirety as if each was incorporated individually.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings.

FIGS. 1A, 1B, 1C and 1D are condensed schematics illustrating the main features of a generalized XNTP and their use in Sequencing by Expansion (SBX).

FIG. 2 is a schematic illustrating more details of one embodiment of an XNTP.

FIG. 3 is a schematic illustrating one embodiment of an Xpandomer passing through a biological nanopore.

FIG. 4 is a gel showing primer extension products.

FIG. 5 is a gel showing primer extension products.

FIGS. 6A and 6B are histogram displays of populations of aligned reads of nanopore-derived sequences.

FIGS. 7A and 7B are histogram displays of populations of aligned reads of nanopore-derived sequences.

FIG. 8 is a gel showing primer extension products.

FIG. 9 is a gel showing primer extension products.

FIG. 10 is a gel showing primer extension products.

FIG. 11 is a gel showing primer extension products.

FIG. 12 is a gel showing primer extension products.

FIG. 13 is a gel showing primer extension products.

FIG. 14 is a gel showing primer extension products.

FIG. 15 illustrates a series of gels which show primer extension products from various exemplary PEM compounds and, in particular, PEM compounds 277 (Lane 2), 51 (Lane 3), 73 (Lane 4), 114 (Lane 5), 311 (Lane 6), 301 (Lane 7), 331 (Lane 8), 273 (Lane 9), and 367 (Lane 10) (according to the compound numbers set forth within Table 1, herein).

### DETAILED DESCRIPTION OF THE DISCLOSURE

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of” or “exactly one of” “Consisting essentially of,” when used in the claims, shall have its ordinary meaning as used in the field of patent law.

As used herein, the terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The present disclosure may be understood more readily by reference to the following detailed description of embodiments of the disclosure and the Examples included herein. Unless otherwise explained, all technical and scientific terms used herein have the same meaning as commonly understood by one of ordinary skill in the art to which this disclosure belongs.

In one aspect of the present disclosure, the present disclosure provides compounds, such as PEM compounds, having Formula (I):

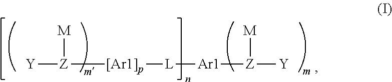

or a solvate, hydrate, tautomer, chelate or salt thereof,
wherein
a is 0 or an integer ranging from 1-4;
a′ is 0 or an integer ranging from 1-4;
m is 1, 2 or 3;
m′ is 1, 2 or 3;
n is 0, 1 or 2;
p is 0, 1 or 2;
Z is a 5-membered or 6-membered heteroaromatic ring including one, two, three, or four heteroatoms selected from O, N, or S, where each Z may be independently substituted with one or more halogens and/or one or more C₁-C₄ alkyl groups;
L is a linking group;
M is, at each occurrence, independently selected from hydrogen, halogen and C₁-C₄ alkyl;
Ar1 is, at each occurrence, independently selected from optionally substituted phenyl, pyridine, bipyridine, tripyridine, pyrazine, pyridazine, furan, dibenzofuran, thiophene, pyrrole, selenophene, naphthalene, fluorene, phenanthrene, cinnoline, phthalazine, quinazoline, quinoxaline, naphthyridine, phenanthroline, purine, and carbazole,
   wherein substituents for Ar1 are, at each occurrence, independently selected from halogen, —OH, —CN, —NO₂, C₁—C ₆ alkyl, C₁-C₆ haloalkyl, C₁-C₆ heteroalkyl, C₁-C₆ cycloloalkyl, —OR⁰, —CONH₂, —C(O)NR¹R^{1′}, —C(O)(CH₂)ₐNR¹R^{1′}, —NR¹R^{1′}, —NR¹C(O)R³, —C(O)SR³, —COR³, —CO(CH₂)ₐOC(O)R³, —OC(O)R³, —C(O)OR³, —C—O—R³, mercaptan, —R⁴—H, —SOR¹, —S(O)₂R¹, —S(O)₂NR¹R^{1′}, —CH₂—NR'S(O)₂R³, —NR¹S(O)₂R³, and —C(CH₃)═N-(phenyl)-O—CH₂—C—CH;
R⁰ is, at each occurrence, independently selected from C₁-C₆ alkyl, C₁-C₆ haloalkyl, C₂-C₆ alkenyl, C₂-C₆ alkynyl, substituted or unsubstituted cycloalkyl, substituted or unsubstituted heterocyclyl, substituted or unsubstituted aryl, and substituted or unsubstituted heteroaryl;
R¹ and R^{1′} are, at each occurrence, independently selected from H, hydroxyl, C₁-C₆ alkyl, C₁-C₆ haloalkyl, C₁-C₂₀ heteroalkyl, C₁-C₁₀ heteroalkyl-NH₂, C₂-C₆ alkenyl, C₂-C₆ alkynyl, substituted or unsubstituted cycloalkyl, substituted or unsubstituted heterocyclyl (e.g., substituted with one or more R³ groups), substituted or unsubstituted aryl, substituted or unsubstituted arylalkyl, and substituted or unsubstituted heteroaryl, benzyl, —C(═NH)NH₂, —CH₂CO₂R⁰, —C(H)(CHₐ(COOH))₂, —CH₂C(O)NHCH₂CO₂H, —CH₂CH₂OH, —CH₂CH₂NHC(O)R³, —(CH₂)ₐC(O)NH(CH₂)ₐC(O)OR³, —(CH₂)ₐ—CN, spermine, —(CH₂)ₐ—NH₂, —C(O)R³, —(CH₂)ₐC(O)OR³, —(CH₂)ₐOR³, —C(H)((CH₂)ₐOH)_{a′}, —CHCH(OH)(CH₂)ₐOH, —(CH₂)ₐOH, C₁-C₆—C(O)OH, —(CH₂)ₐ-heterocycle which may be substituted (e.g., such as with an R³ group) or unsubstituted,

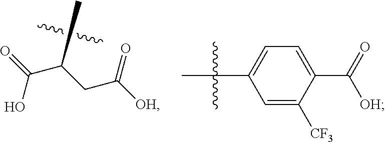

and
wherein R¹ and R^{1′} together form a substituted or unsubstituted heterocyclic ring, including, but not limited to, azetidine, pyrrolidine, piperidine, piperazine, morpholine,

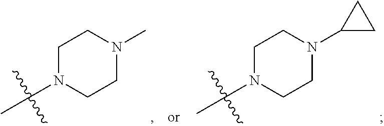

R² is, at each occurrence, independently selected from C₂-C₆alkyl, C₁-C₆ haloalkyl, C₁-C₆ heteroalkyl, or substituted or unsubstituted cycloalkyl, substituted or unsubstituted heterocyclyl, substituted or unsubstituted aryl, substituted or unsubstituted heteroaryl, and substituted or unsubstituted haloalkoxy;
R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine;
R⁴ is, at each occurrence, independently selected from one or more heteroatom interrupted alkylene wherein the heteroatom is O, S, NH, or a combination thereof, Y is, at each occurrence, independently selected from Ar2, —C(O)—Ar2, —(CH₂)ₐAr2, —(CH₂)₃PO(OEt)₂, or —CH₂CO₂Me;
Ar2 is, at each occurrence, independently selected from (i) a substituted or unsubstituted 5- and 6-membered monocyclic aromatic or heteroaromatic ring; (ii) a substituted or unsubstituted 9- and 10-membered fused bicyclic rings comprising two monocyclic rings together, where at least one of the two monocyclic rings is an aromatic or a heteroaromatic ring; and (iii) a substituted or unsubstituted 13- and 14-membered fused tricyclic rings comprising three monocyclic rings together, where at least one of the three monocyclic rings is an aromatic or a heteroaromatic ring;
wherein each Ar2 is independently substituted with G¹, G², G³, G⁴ and G⁵, wherein: when Ar2 is monosubstituted, G¹ is, at each occurrence, independently selected from oxo, C₁-C₆ alkyl, C₁-C₆ haloalkyl, -E-O—R³, -E-C(R¹)(R^{1′})(R³), —NH₂, —NO₂, —SO₃R³, —SO₃O⁻, —SO₃N(H)(R¹), -E-C(O)R³, -E-CO₂H, —B(OH)₂, —C(O)NR¹R^{1′}, -E-PO(OR¹)₂, and aryl substituted with G², G³, G⁴ and G⁵;
G², G³, G⁴ and G⁵ are, at each occurrence, independently selected from absent or selected from the groups comprising, halogen, —CN, —NO₂, —SO₃R³, C₁-C₆ alkyl, C₁-C₆ haloalkyl, -E-O—R³, -E-(CH₂)ₐC(O)R³, -E-CO₂H, -E-CHO, -E-C(O)R³, -E-C(O)NH(OH), -E-C(O)NHR¹, -E-C(O)N(H)C(H)(R¹)(R^{1′}), -E-C(O)N(R¹)((CH₂)ₐP(O)(O)OH)₂, -E-C(O)N(R¹)((CH₂)ₐSO₃H), -E-C(O)NR¹R^{1′}, -E-NR¹R^{1′}, -E-OR², —C(O)-heterocycloalkyl (where the heterocycloalkyl is substituted or unsubstituted), —C(O)—N(H)—CH(COOH)((CH₂)ₐ-heteroaryl) (where heteroaryl is substituted or unsubstituted), wherein E is, at each occurrence, independently selected from a direct bond, and C₁-C₆ alkylene.

In some embodiments, Z is not a triazole. In some embodiments, in some embodiments, Z is not a 1,2,3-triazole or a 1,2,4-triazole.

In some embodiments, Z is a diazole (e.g., imidazole, pyrazole, pyrimidine, pyridazine, pyrazine). In other embodiments, Z is pyrrole, thiophene, or triazole (e.g., 1,2,3-triazole; 1,2,4-triazole). In yet other embodiments, Z is thiazole. In yet other embodiments, Z is isoxazole. In yet further embodiments, Z is a tetrazole.

In yet further embodiments, Z is selected from imidazole, thiazole, isoxazole.

In some embodiments, each Z is independently substituted with one “M” moiety. In other embodiments, each Z is independently substituted with two “M” moieties. In yet other embodiments, each Z is independently substituted with three “M” moieties. In some embodiments, one or more of the compounds having Formula (I) may be polymerized (e.g., via cationic polymerization, such as by incubating one or more PEMs with KCl). In some embodiments, the present disclosure provides a composition including two or more molecules of the compounds having any one of Formulas (I), (IA), (IB), (IC), and/or (ID).

In some embodiments, the compounds of Formula (I) have any one of Formulas (IA) or (IB).

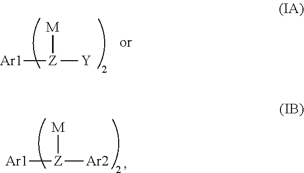

or a solvate, hydrate, tautomer, chelate or salt thereof,
wherein Ar1, Ar2, M, Z, and Y are as defined above. In some embodiments, Z is not a triazole. In some embodiments, in some embodiments, Z is not 1,2,3-triazole or 1,2,4-triazole. In other embodiments, Z is a triazole. In some embodiments, one or more of the compounds having any one of Formulas (IA) or (IB) may be polymerized (e.g., via cationic polymerization, such as by incubating one or more PEMs with KCl). In some embodiments, the present disclosure provides a composition including two or more molecules of the compounds having any one of Formulas (IA) or (IB).

In some embodiments, the compounds of Formula (I) have Formula (IC):

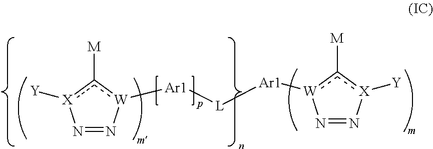

or a solvate, hydrate, tautomer, chelate or salt thereof,
wherein:
W is N when X is C or W is C when X is N;
[symbol] is a single or double bond, wherein the double bond begins at whichever of W or
X is carbon; and
m, m′, n, p, Ar1, L, and Y are as defined above.

In some embodiments, one or more of the compounds having Formula (IC) may be polymerized (e.g., via cationic polymerization, such as by incubating one or more PEMs with KCl). In some embodiments, the present disclosure provides for a composition including two or more molecules of the compounds having Formula (IC).

In some embodiments, the compounds having Formula (IC) have the structure of Formula (ID):

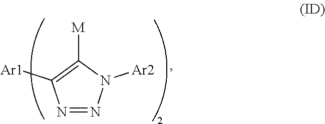

wherein Ar1, Ar2, and M are as defined above.

In some embodiments, one or more of the compounds having Formula (ID) may be polymerized (e.g., via cationic polymerization, such as by incubating one or more PEMs with KCl). In some embodiments, the present disclosure provides a composition including two or more molecules of the compounds having Formula (ID).

As used in the specification and appended claims, unless specified to the contrary, the following terms have the meaning indicated.

Certain chemical groups named herein are preceded by a shorthand notation indicating the total number of carbon atoms that are to be found in the indicated chemical group. For example; C₁-C₄ alkyl, which may alternatively be written as C₁₋₄ alkyl, describes an alkyl group having at least one and up to as many as 4 carbon atoms, while C₄-C₁₂ cycloalkylalkyl (which likewise may be written as C₄₋₁₂ cycloalkylalkyl) describes a cycloalkyl group having a total of 4 to 12 carbon atoms. The total number of carbons in the shorthand notation does not include carbons that may exist in substituents of the group described. As examples, C₁-C₆ alkyl refers to an alkyl radical containing one to six carbon atoms; C₁-C₆ haloalkyl refers to a haloalkyl radical containing one to six carbon atoms; C₁-C₆ alkylene refers to an alkylene diradical containing one to six carbon atoms.

In addition to the foregoing, as used in the specification and appended claims, unless specified to the contrary, the following terms have the meaning indicated:

As used herein, the term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀means one to ten carbons). An “alkyl” is not cyclized. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-butadienyl, 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen atom (—O—).

As used herein, the term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen, phosphorus, and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. A heteroalkyl is not cyclized. Examples include, but are not limited to: CH₂—CH₂—O—CH₃, CH₂—CH₂—NH—CH₃, CH₂—CH₂—N(CH₃)—CH₃, CH₂—S—CH₂—CH₃, —CH₂—O—CH₃, S(O)—CH₃, CH₂—CH₂—S(O)₂—CH₃, CH═CHO—CH₃, Si(CH₃)₃, CH₂—CH═N—OCH₃, CH═CH—N(CH₃)—CH₃, —O—CH₃, —O—CH₂—CH₃, and CN. Up to two heteroatoms may be consecutive, such as, for example, CH₂—NH—OCH₃.

“Alkenyl” refers to a straight or branched hydrocarbon chain radical group consisting solely of carbon and hydrogen atoms, containing at least one double bond, optionally having an indicted number of carbons, e.g., from two to twelve carbon atoms, or two to eight carbon atoms, or two to six carbon atoms, or two to four carbon atoms, and which is attached to the rest of the molecule by a single bond, e.g., ethenyl, prop-1-enyl, but-1-enyl, pent-1-enyl, penta-1,4-dienyl, and the like.

“Alkynyl” refers to a straight or branched hydrocarbon chain radical group consisting solely of carbon and hydrogen atoms, containing at least one triple bond, optionally having an indicated number of carbons, e.g., having from two to twelve carbon atoms, or two to eight carbon atoms, or two to six carbon atoms, or two to four carbon atoms, and which is attached to the rest of the molecule by a single bond, e.g., ethenyl, prop-1-enyl, but-1-enyl, pent-1-enyl, penta-1,4-dienyl, and the like. “Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. Likewise, “haloalkenyl” refers to an alkenyl radical, as defined herein, that is substituted by one or more halo radicals, as defined herein, and “haloalkynyl” refers to an alkynyl radical, as defined herein, which is substituted by one or more halo radicals, as defined herein.

“Alkylene” or “alkylene chain” refers to a straight or branched divalent hydrocarbon chain linking the rest of the molecule to a radical group, consisting solely of carbon and hydrogen, containing no unsaturation, and optionally having an indicated number of carbon atoms. Examples are methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. In analogy to alkyl groups, unsaturation may be introduced into an alkylene chain, to provide an unsaturated alkylene chain. If unsaturation is introduced into an alkylene chain, the resulting group may be referred to as an unsaturated alkylene group or chain, where unsaturated alkylene chains are commonly known as alkenylene groups (having at least one carbon-carbon double bond) and alkynylene groups (having at least one carbon-carbon triple bond). In one embodiment, and when specified, the alkylene chains in compounds of the present disclosure may be, or include, unsaturated alkylene chains.

“Alkenylene” or “alkenylene chain” refers to a straight or branched divalent hydrocarbon chain linking the rest of the molecule to a radical group, consisting solely of carbon and hydrogen, containing at least one double bond, and optionally having an indicated number of carbon atoms, e.g., from two to twelve carbon atoms. Examples of alkenylene groups are ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain.

“Aryl” refers to a ring system radical comprising at least 5 ring atoms, optionally comprising 1-6 hetero ring atoms selected from O, S and N, and at least one aromatic ring. A 5-membered monocyclic aromatic ring contains 5 ring atoms selected from carbon and heteroatoms, while a 6-membered monocyclic aromatic ring contains 6 ring atoms selected from carbon and heteroatoms. Exemplary monocyclic aromatic rings having 5 members is pyrrole and having six-members is pyridine. The aryl radical may be, e.g., a monocyclic, bicyclic, tricyclic, or tetracyclic ring system, which may include fused or bridged ring systems. Carbocyclic aryl radicals contain only carbon at the ring atoms, where examples include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In one embodiment, aryl is phenyl or naphthyl, and in another embodiment is phenyl. When the aryl radical includes non-carbon ring atoms, e.g., oxygen, sulfur, and nitrogen, the aryl group may be referred to as a heteroaryl group. The heteroaryl radical may be, e.g., a monocyclic, bicyclic, tricyclic, or tetracyclic ring system, which may include fused or bridged ring systems. The nitrogen, carbon, or sulfur atoms in the heteroaryl radical may be optionally oxidized; the nitrogen atom may be optionally quaternized.

“Arylalkyl” groups are alkyl, alkenyl or alkynyl groups as defined above in which a hydrogen atom thereof is replaced with an aryl group as defined above. Representative aralkyl groups include benzyl (—CH₂phenyl), phenylethyl (—CH₂CH₂phenyl) and phenylethylene (—CH═CH-phenyl) groups and fused (cycloalkylaryl)alkyl groups such as 4-ethyl-indanyl. Aralky groups can be substituted on the aryl moiety, the alkyl, alkenyl or alkynyl moiety, or both.

“Fused” refers to a ring system which contains fusion between rings, where fusion refers to the rings sharing two adjacent ring atoms. Fused rings that contain two 5- and/or 6-membered monocyclic rings fused together refers to bicyclic ring systems where each ring is monocyclic and independently has either 5 or 6 ring atoms, and the two rings are fused in that they share two ring atoms. For example, naphthalene is a 10-membered fused ring system formed from two 6-membered monocyclic rings (benzene) fused together. Naphthalene is bicyclic in that it contains two (bi=2) rings. As another example, 1,3-benzothiazole which is a 9-membered fused ring system formed from one 6-membered ring (benzene) and one 5-membered ring (1,3-thiazole) fused together. 1,3-benzothiazole is bicyclic in that it contains two rings.

“Carbocyclyl” refers to a stable 3- to 18-membered aromatic or non-aromatic ring radical which consists of 3 to 18 carbon atoms. Unless stated otherwise specifically in the specification, the carbocyclyl radical may be a monocyclic, bicyclic, tricyclic, or tetracyclic ring system, which may include fused or bridged ring systems, and may be partially or fully saturated. Non-aromatic carbocyclyl radicals include cycloalkyl, while aromatic carbocyclyl radicals include aryl.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl and cyclooctyl. Polycyclic radicals include, for example, adamantyl, norbornyl, decalinyl, 7,7-dimethyl-bicyclo-[2.2.1]heptanyl, and the like.

“Heterocyclyl” refers to a stable 3- to 18-membered aromatic or non-aromatic ring radical which consists of two to twelve carbon atoms and from one to six heteroatoms selected from the group consisting of nitrogen, oxygen, and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of non-aromatic heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, pyrazolopyrimidinyl, quinuclidinyl, thiazolidinyl, tetrahydrofuryl, trioxanyl, trithianyl, triazinanyl, tetrahydropyranyl, thiomorpholinyl, thiamorpholinyl, 1-oxo-thiomorpholinyl, and 1,1-dioxo-thiomorpholinyl.

Optionally, although only when specified, each of alkyl, alkenyl, alkylene, alkenylene, carbocyclyl, cycloalkyl, aryl, heterocyclyl and heteroaryl in PEM compounds of the present disclosure may be substituted by one or more unsubstituted (e.g., an alkyl substituent on an alkyl group is not further substituted, i.e., the alkyl substituent is unsubstituted alkyl) substituents selected from the group consisting of alkyl, alkenyl, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R_{b}—ORₐ, —R_{b}—OC(O)—Rₐ, —R_{b}—N(Rₐ)₂, —R_{b}—C(O)Rₐ, —R_{b}—C(O)ORₐ, —R_{b}—C(O)N(Rₐ)₂, —R_{b}—N(Rₐ)C(O)OR_{c}, —R_{b}—N(Rₐ)C(O)R_{c}, —R_{b}—N(Rₐ)S(O)ₜR_{c} (where t is 1 to 2), —R_{b} N═C(ORₐ)Rₐ, —R_{b}—S(O)ₜOR_{c} (where t is 1 to 2), —R_{b}—S(O)ₛR_{c} (where s is 0 to 2), and —R_{b}—S(O)ₜN(Rₐ)₂ (where t is 1 to 2) where each Rₐ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R_{b} is independently a direct bond or a straight or branched alkylene or alkenylene chain; and each R_{c} is alkyl, alkenyl, haloalkyl, cycloalkyl, cycloalkylalkyl, aryl, aralkyl, heterocyclyl, heterocyclylalkyl, heteroaryl or heteroarylalkyl. In some embodiments, substituents may independently be selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, cyanate, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an ether, amino (e.g. a mono-substituted amino group or a di-substituted amino group), and protected derivatives thereof. Any of the above groups may include one or more heteroatoms, including O, N, or S. For example, where a moiety is substituted with an alkyl group, that alkyl group may comprise a heteroatom selected from O, N, or S (e.g. —(CH₂—CH₂—O—CH₂—CH₃)).

“Amino” refers to the —NH₂ radical. “Cyano” refers to the —CN radical. “Hydroxy” refers to the —OH radical. “Nitro” refers to the —NO₂ radical. “Oxo” refers to the ═O substituent. “Thioxo” refers to the ═S substituent. “Trifluoromethyl” refers to the —CF₃ radical. “Trifluoromethoxy” refers to the —OCF₃ radical. Mercaptan, also known as thiol, refers to the —SH radical.

“Acyl” refers to a radical —C(O)R, which may also be written as —C(═O)R, wherein R is alkyl, aralkyl, carbocyclyl, aryl, heteroaryl, or heterocyclyl. For example, when R is methyl, the acyl group may be referred to as acetyl.

“Alkoxy” refers to a radical of the formula —OR where R is an alkyl or haloalkyl radical. In one embodiment, the alkoxy radical contains up to six carbon atoms. Representative alkoxy groups include methoxy and ethoxy. An alkoxy that is substituted with halo may be called herein a haloalkoxy, which includes for example trifluoromethoxy, trichloromethoxy and the like.

“Heteroalkenylene” or “heteroalkenylene chain” refers to a straight or branched divalent hydrocarbon chain linking the rest of the molecule to a radical group, consisting of carbon and hydrogen and at least one heteroatom selected from N, O, and S.

“Haloalkoxy” refers to an alkoxy radical that is substituted by one or more halo radicals, as defined above, e.g., trifluoromethoxy, difluoromethoxy, trichloromethoxy, 2,2,2-trifluoroethoxy, 3-bromo-2-fluoropropyloxy, and the like. The alkoxy part of the haloalkoxy radical may be optionally substituted as defined above for an alkoxy group.

“N-heterocyclyl” refers to a heterocyclyl radical containing at least one nitrogen. An N-heterocyclyl radical may be optionally substituted as described above for heterocyclyl radicals.

“Heterocyclylalkyl” refers to a radical of the formula —R_{b}Rh where R_{b} is an alkylene chain as defined above and Rh is a heterocyclyl radical as defined above, and if the heterocyclyl is a nitrogen-containing heterocyclyl, the heterocyclyl may be attached to the alkyl radical at the nitrogen atom. The alkylene chain of the heterocyclylalkyl radical may be optionally substituted as defined above for an alkylene chain. The heterocyclyl part of the heterocyclylalkyl radical may be optionally substituted as defined above for a heterocyclyl group.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical may be optionally substituted as described above for heteroaryl radicals.

“Heteroarylalkyl” refers to a radical of the formula —R_{b}Rᵢ where R_{b} is an alkylene chain as defined above and Rᵢ is a heteroaryl radical as defined herein. The heteroaryl part of the heteroarylalkyl radical may be optionally substituted as defined herein for a heteroaryl group. The alkylene chain part of the heteroarylalkyl radical may be optionally substituted as defined herein for an alkylene chain. Likewise, an arylalkyl group refers to a heteroarylalkyl group wherein the heteroaryl portion is replaced with the corresponding carbocyclic aryl group, i.e., heteroatoms are replaced with carbon, with adjustment as necessary for hydrogen substitution.

“Hydroxyalkyl” refers to a radical of the formula —R_{b}OH where R_{b} is an alkylene chain as defined herein. The —OH (hydroxyl a.k.a. hydroxy) group can be attached to any carbon in the alkylene chain. The alkylene chain part of the heteroarylalkyl radical may additionally be optionally substituted as defined above for an alkylene chain.

The PEM compounds described herein having acidic or basic groups may generally be used as the free acid or free base. Alternatively, the PEM compounds having acidic or basic groups may be used in the form of salts, e.g., acid or base addition salts. Acid addition salts of the free amino compounds may be prepared by methods well known in the art and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “salt” of the PEM compounds described herein is intended to encompass any and all salt forms.

The PEM compounds of the present disclosure may be in the form of a chelate. A chelate refers to a compound containing an organic ligand (such as a triazole-Ar group) bonded to a central metal atom at two or more points.

With regard to stereoisomers, the PEM compounds described herein may have one or more chiral (or asymmetric) centers and may thus give rise to enantiomers, diastereomers, and other stereoisomeric forms that may be defined, in terms of absolute stereochemistry, as (R)- or (S)-. When the compounds described herein contain olefinic double bonds or other centers of geometric asymmetry, and unless specified otherwise, it is intended that the compounds include both E and Z geometric isomers (e.g., cis or trans). Likewise, unless otherwise indicated, all possible isomers, as well as their racemic and optically pure forms, and all tautomeric forms are also intended to be included. It is therefore contemplated that various stereoisomers and mixtures thereof include “enantiomers,” which refers to two stereoisomers whose molecules are non-superimposable mirror images of one another. Thus, the compounds may occur in any isomeric form, including racemates, racemic mixtures, and as individual enantiomers or diastereomers.

Furthermore, some of the crystalline forms of the PEM compounds may exist as polymorphs, which are contemplated herein. In addition, some of the PEM compounds may also form solvates with water or other organic solvents. Such solvates are similarly included within the scope of the compounds described herein.

As one of skill in the art would appreciate, any of the aforementioned compounds may incorporate radioactive isotopes. Accordingly, also contemplated is use of isotopically labeled compounds identical to those described herein, wherein one or more atoms are replaced by an atom having an atomic mass or mass number different from the atomic mass or mass number usually found in nature. Examples of isotopes that can be incorporated into these compounds include isotopes of hydrogen, carbon, nitrogen, oxygen, phosphorous, fluorine and chlorine. Thus, reference to an element, such as hydrogen (H) or carbon (C), is intended to encompass all isotopes of the same. For example, the designation C (carbon) includes ¹²C, ¹³C, or ¹⁴C and mixtures thereof, while H (hydrogen) includes ¹H, ²H and ³H and mixtures thereof, and O (oxygen) includes ¹⁶O and ¹⁸O and mixtures thereof, and N (nitrogen) includes ¹⁴N and ¹⁵N and mixtures thereof, etc. for other atoms. Isotopically labeled PEM compounds may be useful in tracking PEM compounds or portions thereof during their use in assays etc.

In some embodiments, the PEM compounds of any one of Formulas (I), (IA), (IB), (IC), and (ID) include an Ar1 moiety, where, in some embodiments, Ar1 is an aryl group, also referred to as an aromatic moiety. The aromatic moiety may be a carbocyclic or heterocyclic aromatic moiety, where each of the aromatic ring atoms are carbon in a carbocyclic aromatic moiety, while at least one of the aromatic ring atoms is nitrogen, oxygen, or sulfur in a heterocyclic aromatic moiety.

In some embodiments, Ar1 includes 1-6 rings, where up to six of the ring atoms may be selected from oxygen, sulfur, and nitrogen, with the remainder being carbon atoms. Optionally, the Ar1 moiety may comprise 1-5 rings, where up to five of the ring atoms may be selected from oxygen, sulfur, and nitrogen. In other embodiments, the Ar1 group may comprise 1-4 rings, where up to four of the ring atoms may be selected from oxygen, sulfur, and nitrogen. As yet other embodiments, the Ar1 moiety may comprise 1-3 rings, where up to three of the ring atoms may be selected from oxygen, sulfur, and nitrogen. As a further example, Ar1 may comprise 1-2 rings, where up to three of the ring atoms may be selected from oxygen, sulfur, and nitrogen. As described herein, each ring may independently be a five-membered ring, i.e., five ring atoms form the ring, or a six-membered ring, or a seven-membered ring, while in some embodiments each of the rings is either a five- or six-membered ring.

In some embodiments, A1 is a carbocyclic aromatic moiety. In some embodiments, carbocyclic moiety may contain one (e.g., benzene) or two (e.g., naphthalene, azulene) or three (e.g., acenaphthylene, fluorene) or four (e.g., fluoranthene, aceanthrylene) or five (e.g., pentacene, picene) or six (e.g., hexacene) aromatic rings, where for convenience the Ar1 group may be exemplified herein by naming the unsubstituted version thereof (e.g., benzene) although in compounds of the present disclosure the Ar1 group is the corresponding radical, e.g., when m is 2 and Ar1 is otherwise unsubstituted, two ring hydrogens replaced with triazole groups. For example, the Ar1 may be a monocyclic carbocyclic moiety, i.e., phenyl, also referred to as a C₆ aromatic moiety. As another example, Ar1 may be a bicyclic carbocyclic moiety, e.g., naphthyl, which is a C₁₀ aromatic moiety.

Non-limiting examples of aromatic Ar1 moieties include heterocyclic aromatic moieties, which may also be referred to as a heteroaryl group. In some embodiments, any heterocyclic moiety may contain one or two or three or four or five or six aromatic rings, in addition to containing 1 or 2 or 3 or 4 or 5 or 6 heteroatoms, i.e., atoms other than carbon, selected from nitrogen, sulfur and oxygen atoms. Optionally, the heteroatom, if present, is nitrogen, oxygen, sulfur, or selenium. For example, the aromatic moiety may be a monocyclic heterocyclic moiety, e.g., pyridinyl, which is a six-membered C₅ aromatic moiety, or pyrazinyl, which is a six-membered C₄ aromatic moiety. As another example, the aromatic moiety may be a bicyclic heterocyclic moiety, e.g., quinolinyl or isoquinolinyl, which are ten-membered C₉ aromatic moieties, or 1,5-naphthylidinyl, 2,6-naphthylidinyl or 2,7-naphthylidinyl, which are exemplary ten-membered C₈ aromatic moieties.

In some embodiments, the heteroaryl groups are aromatic ring compounds containing 5 or more ring members, of which, one or more is a heteroatom such as, but not limited to, N, O, S, and Se. In some embodiments, an heteroaryl group designated as a C₂-heteroaryl can be a 5-membered ring with two carbon atoms and three heteroatoms, a 6-membered ring with two carbon atoms and four heteroatoms and so forth. Likewise, a C₄-heteroaryl can be a 5-membered ring with one heteroatom, a 6-membered ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, iso quinolinyl, tetrahydroquinolinyl, tetrahydroisoquinolinyl, quinoxalinyl, and quinazolinyl groups. Thus, the terms “heteroaryl” and “heteroaryl groups” include fused ring compounds such as wherein at least one ring, but not necessarily all rings, are aromatic, including tetrahydroquinolinyl, tetrahydroisoquinolinyl, indolyl and 2,3-dihydro indolyl.

In some embodiments, when m is 2, such that Ar1 is necessarily substituted with two Z—Ar2 moieties (as in Formulas (I), (IA), and (IB)) or two triazole-Ar2 moieties (as in Formulas (IC) and (ID), any two carbons of the Ar1 moiety may be substituted with one of these two Z—Ar2 moieties or triazole-Ar2 moieties. For example, when Ar1 is substituted benzene, Ar1 may be substituted in the ortho, meta or para positions, as shown below, where k designates where the substitution may occur on the aromatic moiety:

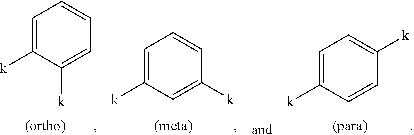

In other embodiments, when Ar1 is substituted naphthalene and m is 2, Ar1 may be substituted at any two naphthyl carbon atoms, where the following structures show the substitution options, with k showing where the “Z” group substitution (e.g., Z—Ar2 or triazole-Ar2) may occur on the aromatic moiety:

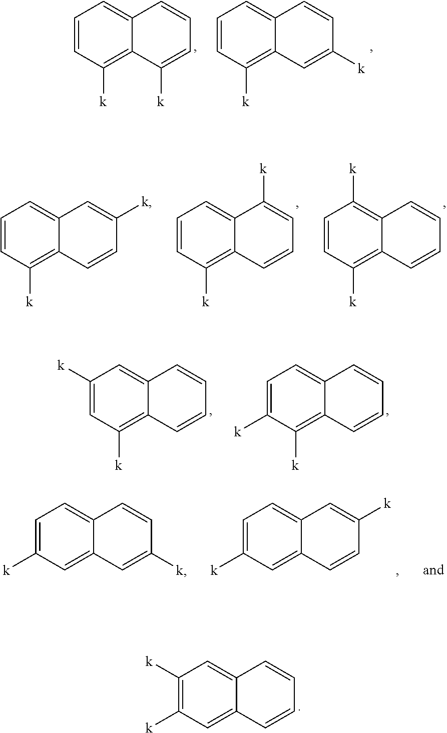

The preceding examples illustrated a “Z” group (e.g., triazole) on Ar1 using carbocyclic Ar1 moieties as an illustrative Ar1 moiety. The same principle applies, however, to “Z” group substitutions (e.g., triazole substitutions) on heterocyclic aromatic Ar1 groups. For example, when Ar1 is a substituted pyridine and m is 2, the two “Z” groups (e.g., triazole groups) of Z—Ar2 (e.g., triazole-Ar2) may be located at any of the following locations on the pyridine ring, where k is used to designate the positions where “Z” groups (e.g., triazole groups) may be located:

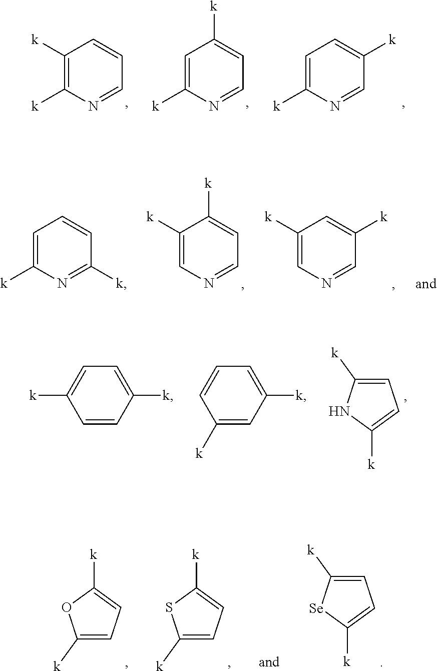

In some embodiments, Ar1 is a monocyclic heteroaromatic structure selected from

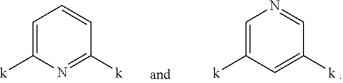

wherein the rings of the “Z” group (e.g., triazole rings) are substituted at positions k on Ar1.

In other embodiments, Ar1 is a monocyclic carbocyclic structure selected from:

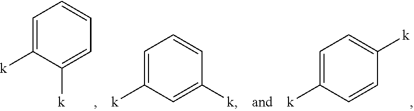

wherein the rings of the “Z” group (e.g., triazole rings) are substituted at positions k on Ar1.

In yet other embodiments, Ar1 is a bicyclic carbocyclic structure selected from:

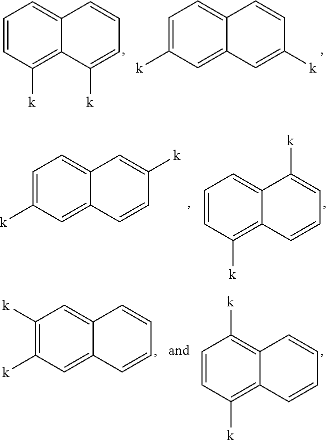

wherein the rings of the “Z” group (e.g., triazole rings) are substituted at positions k on Ar1.

In yet further embodiments, Ar1 is a polycyclic heterocyclic structure having two six-membered rings and one five-membered ring, and one nitrogen ring atom and selected from:

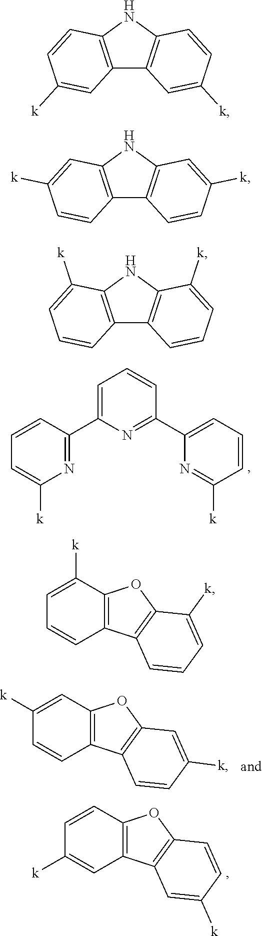

wherein the rings of the “Z” group (e.g., triazole rings) are substituted at positions k on Ar1.

In yet even further embodiments, Ar1 is a polycyclic heterocyclic structure having three six-membered rings and two nitrogen ring atoms and being selected from:

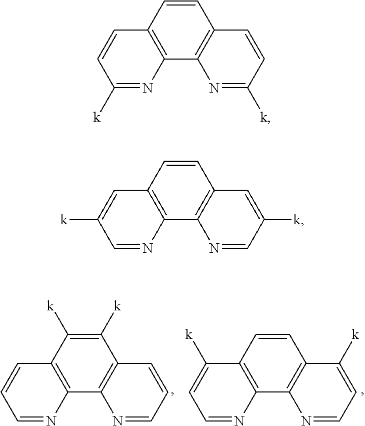

wherein the rings of the “Z” group (e.g., triazole rings) are substituted at positions k on Ar1.

In some embodiments, Ar1 includes both substituted and unsubstituted moieties as described herein. In other embodiments, Ar1 is a substituted aromatic moiety. In yet other embodiments, Ar1 is unsubstituted aromatic moiety. In a substituted Ar1 moiety, one or more hydrogen atoms that would have been bonded to a ring atom has been replaced with a substituent, for example, optionally 1, or 2, or 3, or 4, or 5, or 6 of the hydrogen atoms may be replaced with a substituent (or moiety). A substituent on Ar1 does not refer to the Z—Ar2 (e.g., triazole-Ar2) moiety that is necessarily present when m equals 1, or the two Z—Ar2 (e.g., triazole-Ar2) moieties that are necessarily present when m equals 2, or the three Z—Ar2 (e.g., triazole-Ar2) moieties that are necessarily present when m equals 3.

In some embodiments, a substituent on Ar1 includes atoms selected from deuterium, halogen (F, Cl, Br, I), carbon, nitrogen, oxygen and sulfur, and optionally will also contain hydrogen, and also will contain additional atoms that form a counterion, if present. Deuterium and halide are considered monovalent atoms, while carbon, nitrogen, oxygen, and sulfur, because they are capable of simultaneously forming more than one covalent bond, are considered multivalent atoms. In addition to monovalent atoms, a substituent on Ar1 may have multiple multivalent atoms, e.g., 1-25 multivalent atoms, or 1-22 multivalent atoms, or 1-15 multivalent atoms, or 1-10 multivalent atoms, or 1-5 multivalent atoms, the atoms being optionally selected from carbon, nitrogen, oxygen, and sulfur. Illustrations of substituents with up to 10 multivalent atoms are provided below. Other substituents, including substituents with up to 25 multivalent atoms, are known by analogy to one of ordinary skill in the art.

In some embodiments, a substituent on Ar1 contains 0 multivalent atoms. In this embodiment, a hydrogen bonded to a ring atom is replaced with another monovalent atom, such as deuterium, fluorine, chlorine, bromine, or iodine.

In other embodiments, a substituent on Ar1 contains 1 multivalent atom. In this embodiment, one or more hydrogen atoms bonded to a ring atom of Ar1 are replaced with a single multivalent atom, where open valencies on the multivalent atom are filled with one or more monovalent atoms, examples being hydroxyl (OH), thiol (SH), amino (NH₂), methyl (CH₃) and methylene (═CH₂) including fully or partially halogenated and deuterated version thereof, e.g., CF₃.

In yet other embodiments, a substituent on Ar1 contains 2 multivalent atoms. In these embodiments, one or more hydrogen atoms bonded to a ring atom of Ar1 are replaced with a first multivalent atom which, in turn, is bonded to a second multivalent atom, thus providing a substituent formed from two multivalent atoms, where open valencies on the multivalent atoms are filled with one or more monovalent atoms. Examples of these substituents are well known to one of ordinary skill in the art. Specific examples include ethyl (CH₂CH₃), ethylene (CH═CH₂), ethynyl (C—CH), ethylidene (═CHCH₃), aminomethyl (CH₂NH₂), aminomethylene (═CHNH₂), thiomethylene (═CHSH), hydroxymethylene (═CHOH), hydroxymethyl (CH₂OH), thiomethyl (CH₂SH), N-methylamine (NHCH₃), methylsulfide (SCH₃), methoxy (OCH₃), nitrile (CN), formyl (C(O)H), thioformyl (C(S)H), N-hydroxy (N—OH), hydroxylamine (ONH₂), hydrazine (NH₂NH₂), diazine (N═NH), diazonium (N—N), including fully or partially halogenated and deuterated versions thereof, e.g., OCF₃ and CH₂CD₃.

In yet other embodiments, a substituent on Ar1 contains 3 multivalent atoms. In this embodiment, one or more hydrogen atoms bonded to a ring atom of Ar1 are replaced with a first multivalent atom which, in turn, is bonded directly or indirectly to each of a second and third multivalent atom; thus, the first multivalent atom is bonded to a second multivalent atom, and a third multivalent atom is bonded to either or both of the first and second multivalent atoms, thus providing a substituent formed from three multivalent atoms, where open valencies on the multivalent atoms are filled with one or more monovalent atoms. Examples of these substituents are well known to one of ordinary skill in the art and are provided herein, e.g., nitro, methylketone, carboxyl.

In yet other embodiments, a substituent on Ar1 contains 4 multivalent atoms. In this embodiment, one or more hydrogen atoms bonded to a ring atom of Ar1 are replaced with a first multivalent atom which, in turn, is bonded directly or indirectly to each of a second, third and fourth multivalent atom, thus providing a substituent formed from four multivalent atoms, where open valencies on the multivalent atoms are filled with one or more monovalent atoms. Examples of these substituents are well known to one of ordinary skill in the art and are provided herein, e.g., methylester (CO₂CH₃), N-methylcarboxamide (C(O)NHCH₃) and acetamide (NHC(O)CH₃).

In yet other embodiments, a substituent on Ar1 contains 5 multivalent atoms. In this embodiment, one or more hydrogen atoms bonded to a ring atom of Ar1 are replaced with a first multivalent atom which, in turn, is bonded directly or indirectly to each of a second, third, fourth and fifth multivalent atom, thus providing a substituent formed from five multivalent atoms, where open valencies on the multivalent atoms are filled with one or more monovalent atoms. Examples of these substituents are well known to one of ordinary skill in the art and are provided herein, e.g., ethylester (CO₂CH₂CH₃), S-ethylcarbothioate (C(O)SCH₂CH₃), N-ethylcarboxamide (C(O)NHCH₂CH₃) and N,N-dimethylcarboxamide (C(O)N(CH₃)₂).

In yet other embodiments, a substituent on Ar1 contains 6 multivalent atoms. In this embodiment, one or more hydrogen atoms bonded to a ring atom of Ar1 are replaced with a first multivalent atom which, in turn, is bonded directly or indirectly to each of a second, third, fourth, fifth and sixth multivalent atom, thus providing a substituent formed from six multivalent atoms, where open valencies on the multivalent atoms are filled with one or more monovalent atoms. Examples of these substituents are well known to one of ordinary skill in the art and are provided herein, e.g., N-cyclopropylcarboxamide (C(O)NH— cyclopropyl), N-propylcarboxamide (C(O)NHCH₂CH₂CH₃), N-(2-hydroxyethyl)carboxamide (C(O)NHCH₂CH₂OH) and N-carbamimidocarboxamide (C(O)NHC(═NH)NH₂).

In yet other embodiments, a substituent on Ar1 contains 7 multivalent atoms. In this embodiment, one or more hydrogen atoms bonded to a ring atom of Ar1 are replaced with a first multivalent atom which, in turn, is bonded directly or indirectly to each of a second, third, fourth, fifth sixth and seventh multivalent atom, thus providing a substituent formed from seven multivalent atoms, where open valencies on the multivalent atoms are filled with one or more monovalent atoms. Examples of these substituents are well known to one of ordinary skill in the art and are provided herein, e.g., N-(n-butyl)carboxamide (C(O)NHCH₂CH₂CH₂CH₃), N-(t-butyl)carboxamide (C(O)NHC(CH₃)₃), N,N-diethylcarboxamide (C(O)N(CH₂CH₃)₂), and N-cyclobutylcarboxamide (C(O)NH(cyclobutyl)).

In yet other embodiments, a substituent on Ar1 contains 8 multivalent atoms. In this embodiment, one or more hydrogen atoms bonded to a ring atom of Ar1 are replaced with a first multivalent atom which, in turn, is bonded directly or indirectly to each of a second, third, fourth, fifth sixth, seventh and eighth multivalent atom, thus providing a substituent formed from eight multivalent atoms, where open valencies on the multivalent atoms are filled with one or more monovalent atoms. Examples of these substituents are well known to one of ordinary skill in the art and are provided herein, e.g., N-cyclopentylcarboxamide (C(O)NH(cyclopentyl)), (piperidin-1-yl)methanone (C(O)-piperidin-1-yl) and (morpholin-4-yl)methanone (C(O)-morpholin-4-yl).

In yet other embodiments, a substituent on Ar1 contains 9 multivalent atoms. In this embodiment, one or more hydrogen atoms bonded to a ring atom of Ar1 are replaced with a first multivalent atom which, in turn, is bonded directly or indirectly to each of a second, third, fourth, fifth sixth, seventh, eighth and ninth multivalent atom, thus providing a substituent formed from nine multivalent atoms, where open valencies on the multivalent atoms are filled with one or more monovalent atoms. Examples of these substituents are well known to one of ordinary skill in the art and are provided herein, e.g., di-(iso-propyl)ester (C(O)O(CH(CH₃)₂)₂, di-(n-propyl)ester (C(O)O(CH₂CH₂CH₃)₂), N-cyclohexylcarboxamide (C(O)NH(cyclohexyl)), (4-methylpiperazin-1-yl)methanone (C(O)(4-methylpiperazin-1-yl), 2-(acetylamino)ethylcarboxamide (C(O)NHCH₂CH₂NHC(O)CH₃) and N-phenylcarboxamide (C(O)NH(phenyl)).

In yet other embodiments, a substituent on Ar1 contains 10 multivalent atoms. In this embodiment, one or more hydrogen atoms bonded to a ring atom of Ar1 are replaced with a first multivalent atom which, in turn, is bonded directly or indirectly to each of a second, third, fourth, fifth sixth, seventh, eighth, ninth and tenth multivalent atom, thus providing a substituent formed from ten multivalent atoms, where open valencies on the multivalent atoms are filled with one or more monovalent atoms. Examples of these substituents are well known to one of ordinary skill in the art and are provided herein, e.g., N-benzylcarboxamide (C(O)NHCH₂(phenyl)).

In some embodiments, Ar1 is substituted aryl wherein at least one substituent on Ar1 is selected from halogen, hydroxyl, mercaptan, nitro, and nitrile.

In other embodiments, Ar1 is substituted with at least one substituent is selected from halogen, —OH, —CN, —NO₂, C₁-C₆ alkyl, C₁-C₆ haloalkyl, C₁-C₆ heteroalkyl, C₁-C₆ cycloloalkyl, —OR⁰, —CONH₂, —C(O)NR¹R^{1′}, —C(O)(CH₂)ₐNR¹R^{1′}, —N¹RR^{1′}, —NR¹C(O)R³, —C(O)SR³, —COR³, —CO(CH₂)ₐOC(O)R³, —OC(O)R³, —C(O)OR³, —C—O—R³, mercaptan, —R⁴—H, —SOR¹, —S(O)₂R¹, —S(O)₂NR¹R^{1′}, —CH₂—NR¹S(O)₂R³, —NR'S(O)₂R³, and —C(CH₃)═N-(phenyl)-O—CH₂—C—CH; and where:

R⁰ is, at each occurrence, independently selected from C₁-C₆alkyl, C₁-C₆ haloalkyl, C₂-C₆ alkenyl, C₂-C₆ alkynyl, substituted or unsubstituted cycloalkyl, substituted or unsubstituted heterocyclyl, substituted or unsubstituted aryl, and substituted or unsubstituted heteroaryl;

R¹ and R^{1′} are, at each occurrence, independently selected from H, hydroxyl, C₁-C₆ alkyl, C₁-C₆ haloalkyl, C₁-C₂₀ heteroalkyl, C₁-C₁o heteroalkyl-NH₂, C₂-C₆ alkenyl, C₂-C₆ alkynyl, substituted or unsubstituted cycloalkyl, substituted or unsubstituted heterocyclyl (e.g., substituted with one or more R³ groups), substituted or unsubstituted aryl, substituted or unsubstituted arylalkyl, and substituted or unsubstituted heteroaryl, benzyl, —C(═NH)NH₂, —CH₂CO₂R⁰, —C(H)(CHₐ(COOH))₂, —CH₂C(O)NHCH₂CO₂H, —CH₂CH₂OH, —CH₂CH₂NHC(O)R³, —(CH₂)ₐC(O)NH(CH₂)ₐC(O)OR³, —(CH₂)ₐ—CN, spermine, —(CH₂)ₐ—NH₂, —C(O)R³, —(CH₂)ₐC(O)OR³, —(CH₂)ₐOR³, —C(H)((CH₂)ₐOH)_{a′}, —CHCH(OH)(CH₂)ₐOH, —(CH₂)ₐOH, C₁-C₆—C(O)OH, —(CH₂)ₐ-heterocycle which may be substituted (e.g., such as with an R³ group) or unsubstituted,

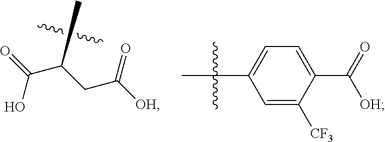

and

wherein R¹ and R^{1′} together form a substituted or unsubstituted heterocyclic ring, including, but not limited to, azetidine, pyrrolidine, piperidine, piperazine, morpholine,

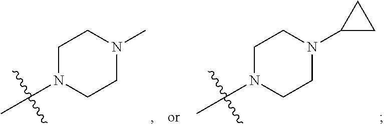

R² is, at each occurrence, independently selected from C₂-C₆ alkyl, C₁-C₆ haloalkyl, C₁-C₆ heteroalkyl, or substituted or unsubstituted cycloalkyl, substituted or unsubstituted heterocyclyl, substituted or unsubstituted aryl, substituted or unsubstituted heteroaryl, and substituted or unsubstituted haloalkoxy;

R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine; and

R⁴ is, at each occurrence, independently selected from one or more heteroatom interrupted alkylene wherein the heteroatom is O, S, NH, or a combination thereof.

In some embodiments, Ar1 is substituted with at least one substituent selected from the group consisting of substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted aryl, substituted or unsubstituted heteroaryl, substituted or unsubstituted arylalkyl, substituted or unsubstituted heteroarylalkyl, substituted or unsubstituted haloalkyl, and substituted or unsubstituted haloalkoxy.

In some embodiments, Ar1 includes an aromatic moiety as described herein, where the aromatic moiety may optionally be substituted as also described herein, which substitution is in addition to being substituted with (Z—Ar2)ₘ groups (e.g., (triazole-Ar2)ₘ groups).

In some embodiments, substituents of Ar1 include, but are not limited to, halide such as fluoride, chloride and bromide, alkyl groups having 1-6 carbon atoms such as methyl and ethyl, haloalkyl groups having 1-6 carbon atoms such as trifluoromethyl, cyano, formyl, and carboxamide.

In other embodiments, substituents of Ar1 include, but are not limited to, nitro (—NO₂), cyano (—CN), carboxylic acid (—COOH, or salts thereof), carboxamide (—C(O)NH₂), C₁-C₆ alkoxy such as methoxy, isonicotinate ester, isonicotinamide, C₁—C ₆alkyl including methyl, C₁-C₆ haloalkyl such as trifluoromethyl, C₁-C₆ heteroalkyl including amides such as —NHC(O)(C₁-C₆ alkyl), —NHC(O)(C₁-C₆ heteroalkyl), —C(O)NH(C₁—C ₆alkyl), —C(O)NH(C₁-C₆ heteroalkyl), —C(O)N(C₁-C₆ alkyl)(C₁-C₆ alkyl), —C(O)N(C₁-C₆ alkyl)(C₁-C₆ heteroalkyl) and —C(O)N(C₁-C₆ heteroalkyl)(C₁-C₆ heteroalkyl) including —NHC(O)CH₃, C(O)NHCH₃, —C(O)N(CH₃)₂, —NHC(O)CH₂CH₃, C(O)NHCH₂CH₃, —C(O)N(CH₃)CH₂CH₃, —C(O)N(CH₂CH₃)₂, —C(O)NH(C₁-C₆cycloalkyl) and —NHC(O)(C₁-C₆cycloalkyl) (e.g., C(O)NH(cyclopropyl), —NHC(O)-cyclopropyl, C(O)NH(cyclohexyl), NHC(O)-cyclohexyl), C(O)NHCH₂CH₂CH₂CH₃, —C(O)NH(C(CH₃)₃), —C(O)NH(CH₂CH₂OH), ketones such as —C(O)(C₁-C₆ alkyl) including —C(O)CH₃, —C(O)(cycloalkyl) including —C(O)-cyclohexyl, and C(O)-(heterocycloalkyl) where the heterocycloalkyl may be, e.g., morpholinyl, piperidinyl, piperazinyl, N-methylpiperazinyl, esters such as —CO₂—(C₁-C₆ alkyl) including —CO₂CH₃, —O₂CH₂CH₃, —CO₂CH₂CH₂CH₃, —CO₂CH₂(CH₃)₂, and thioesters such as C(O)—S—(C₁-C₆ alkyl) including —C(O)—S—CH₃ and —C(O)—S—CH₂CH₃.

In some embodiments, substituents of Ar1 include, but are not limited to, —C(O)—CH₃, —C(O)—NH₂, —C(O)O—CH₂CH₃, —CF₃, —C(O)O-t-butyl, —C(O)—N(CH₃)₂, —C(O)—N(H)CH₃, —C(O)—N(H)CH₂CH₃, —C(O)—N(H)(cycloalkyl), —C(O)—N(H)(CH₂)₄, —C(O)—N(CH₂CH₃)₂, —C(O)—N(H)(t-butyl), —C(O)—N(H)(C6-cycloalkyl), —C(O)—N(H)(CH₂)₃, —C(O)—N(H)(C5-cycloalkyl), —C(O)—N(H)(C4-cycloalkyl), —C(O)—N(H)(phenyl), —C(O)—N(H)(CH₂CH₂)N(H)C(O)—CH₃, —C(O)—N(H)C(NH)(NH₂), —C(O)—N(CH₂CH₂CH₃)₂, —C(O)—N—(C(H)(CH₃)(CH₂CH₃))₂, —C(O)—N(CH₂CH₂CH₂CH₃)₂, —C(O)—N(H)(benzyl), —C(O)—N(H)—CH₂CH₂OH, —C(O)—S—CH₂CH₃, —C(O)N(CH₂)₂, —N(H)S(O)(O)-phenyl, —C(O)N(H)CH₂CH₂CF₃, —C(O)N(H)CH₃, —C(O)OCH₂CH₃, —C(O)—N(H)—CH₂CH₂O—CH₂CH₂O—CH₃, —C(O)—N(H)—(CH₂CH₂O)₈—CH₃, —C(O)N(H)—CH₂—CH₂—CH₂—N(H)—CH₂—CH₂—CH₂—N(H)—CH₂—CH₂—CH₂—NH₂, —C(O)N(H)—CH₂—CH₂—NH₂, —C(O)N(H)C(O)CH₃, —C(O)N(H)—CH₂—CH₃, —S(O)(O)N(H)—CH₂—CH₃, —S(O)(O)N—(CH₂—CH₃)₂, —S(O)(O)N(H)—(C3-cycloalkyl), —S(O)(O)N(H)—(CH₂CH₂)OH, —S(O)(O)OH, —C(O)N(H)C(H)(CH₂OH)₂, —C(O)N(CH₂CH₂OH)₂, —C(O)N(H)CH₂CH₂═CH₂, —C(O)CH₂N(CH₃)(CH₂)(COOH), —S(O)(O)(CH₂CH₃), —S(O)(O)N(H)CH₂—CH(OH)(CH₂OH), —S(O)(O)—NH₂, or —S(O)(O)N(H)CH₂—C(O)(O-t-butyl).

In some embodiments, Ar1 is substituted with at least one moiety selected from —O—(C₁₋₆ alkyl), C₁₋₆ alkyl, C₁₋₆ haloalkyl, —CO₂—C₁₋₆ alkyl, —CONH—C₁₋₆ alkyl, —CONH₂, CN; and —NO₂.

In some embodiments, when n is 1 or 2, the compounds of the present disclosure (such as Formulas (I) and/or (IC)) include a linker, L. In some embodiments, the linker L may be a direct bond. In other embodiments, the linker is not a direct bond, but is instead one or more atoms, particularly atoms selected from carbon, nitrogen, oxygen, sulfur. In yet other embodiments, the linker may be an alkylene group (e.g., C₁-C₆ alkylene), or a substituted alkylene. In some embodiments, the linker may be a heteroalkylene linker, which refers to a substituted or non-substituted alkylene which further includes at least one heteroatom (e.g., 1, 2, 3, or 4 heteroatoms) selected from oxygen, nitrogen, or sulfur within and/or placed at one or more terminal position(s) of the parent chain. In some embodiments, L is a heteroalkylene group of 2 to 10 carbon atoms in length, wherein one or more carbon atoms is replaced with at least one heteroatom selected from oxygen, nitrogen, and sulfur. In some embodiments, L may be a heteroalkylene linker having at least one N, O or S heteroatom, wherein the heteroalkylene may be a straight chain or cyclized and optionally substituted, where exemplary substituents include oxo, —OH, C₁₋₄alkyl and C₁₋₄alkoxy. Non-limiting examples of heteroalkylene linker groups include amide-containing heteroalkylene groups such as —C(O)NH-alkylene- and —C(O)NH-alkylene-NHC(O)—, where alkylene is optionally C₁-C₆ alkylene. Other examples of heteroalkylene groups include ester-containing heteroalkylene groups such as —C(O)O-alkylene- and —C(O)O— alkylene-OC(O)—, where in one embodiment alkylene is unsubstituted C₁-C₆ alkylene, and in another embodiment alkylene is substituted C₁-C₆ alkylene. In some embodiments, the linker is hydrolytically stable, so that it does not decompose, degrade, or otherwise break when the PEM is placed into water.

In some embodiments, the linker L includes from 1 to about 25 atoms excluding hydrogen and halogen from that atomic count, where the linker may optionally be composed of atoms selected from carbon, nitrogen, oxygen and sulfur, in addition to hydrogen and halogen. In some embodiments, the linker has fewer than 25 atoms (excluding hydrogen and halogen), e.g., it includes 1 to about 20 atoms, or 1 to about 15 atoms, or 1 to about 10 atoms, or 1 to about 5 atoms, in each case excluding hydrogen and halogen from that atomic count, where the counted atoms may optionally be selected from carbon, oxygen, nitrogen and sulfur.

In some embodiments, a group “Z” (e.g., a triazole) in a PEM compound of Formula (I) may be substituted in addition to being directly bonded to Ar1 and Ar2. In general, compounds of the present disclosure may optionally be described as including the chemical formula Ar1-Z(M)-Ar2

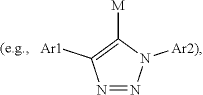

where Ar1,Ar2, and M are as defined herein. Depending on the particular substituent “Z,” Z may be substituted with one or more “M” moieties, such as 1 “M” moiety, 2 “M” moieties, 3 “M” moieties, 4 “M” moieties, etc. In some embodiments, “Z” is a triazole, and the triazole ring is substituted only by Ar1 and Ar2, i.e., M is hydrogen. In other embodiments, Z is substituted with Ar1, Ar2, and one of methyl, ethyl, n-propyl, isopropyl, n-butyl, isobutyl, or sec-butyl. In yet other embodiments, Z is substituted with Ar1, Ar2, and one of fluorine, chlorine, or iodine.

Thus, in one embodiment, the present disclosure provides compounds having Formula (IB):

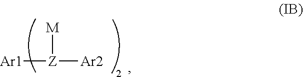

where Ar1, Ar2, and M are as defined herein. When a compound is substituted with more than one “M” moiety, each “M” moiety is independently selected at each occurrence. However, in one embodiment, M is the same atom at each occurrence in a compound of the present disclosure. For example, the present disclosure provides compounds wherein M is hydrogen at each occurrence of M. In another example, the present disclosure provides compounds wherein M is iodide at each occurrence of M. In some embodiments, Z is not a triazole. In some embodiments, Z is not 1,2,3-triazole or 1,2,4-triazole.

In some embodiments, when a compound of the present disclosure includes two, or more than two, —Z(M)-Ar2 groups, the Ar2 moieties may optionally have the same chemical structure at each occurrence. However, when a PEM compound of the present disclosure contains multiple —Z(M)-Ar2 groups, in one embodiment those Ar2 moieties are not necessarily identical to one another, and in fact they may be non-identical. In some embodiments, each Ar2 moiety may differ from one another in terms of the Ar2 ring atoms and/or in terms of the substitution on the Ar2 ring atoms.

For example, if one Ar2 group is phenyl and the other Ar2 group is pyridinyl, then the two Ar2 groups differ in terms of the ring atoms that compose the Ar2 group.

As another example, if both Ar2 groups are phenyl, but one phenyl is substituted with carboxyl while the other phenyl is substituted with methoxy, as in, e.g., 4-(4-(3-(1-(4-methoxyphenyl)-1H-1, 2,3-triazol-4-yl)phenyl)-1H-1,2,3-triazol-1-yl)benzoic acid, then the compound is considered to have two different Ar2 groups.

Yet another example, the two Ar2 groups may be positional isomers of one another, as in when both Ar2 groups are phenyl, and both phenyl rings are substituted with hydroxyl and carboxyl, but the locations of the hydroxyl and/or carboxyl groups are different on the two phenyl rings, e.g., if on one phenyl ring the triazole is located at the 3 position (meta) relative to the carboxyl group while on the other phenyl ring the triazole is located at the 4 position (para) relative to the carboxyl group, then the two Ar2 groups are considered to be positional isomers and non-identical.

In some embodiments, the Ar2 rings are identical in all respects at each occurrence in a compound of the present disclosure. In some embodiments, the Ar2 ring atoms are identical at each occurrence of Ar2, but the substitution on the Ar2 rings is non-identical at each occurrence of Ar2. In some embodiments, the Ar2 ring atoms are non-identical at each occurrence of Ar2, and the substitution on the Ar2 rings may or may not be identical.

In some embodiments, the compounds of Formula (I) include at least one Ar2 moiety. In some embodiments, Ar2 is a monocyclic 6-membered aromatic ring, non-limiting examples of which include phenyl, pyridinyl and pyrazinyl, and where the Ar2 group optionally includes substituents on the ring atoms. In other embodiments, Ar2 is a 5-membered monocyclic aromatic ring, which may optionally be substituted. In yet other embodiments, Ar2 is a 5- or 6-membered aromatic ring, which may optionally be substituted. In yet further embodiments, Ar2 is a 9- or 10-membered fused bicyclic ring comprising two 5- and/or 6-membered monocyclic rings fused together, where at least one of the two monocyclic rings is an aromatic ring. In even further embodiments, Ar2 is a 9- or 10-membered fused bicyclic ring comprising two 5- and/or 6-membered monocyclic rings fused together, where both of the two monocyclic rings are an aromatic ring. In yet even further embodiments, Ar2 is a 13- and 14-membered fused tricyclic ring comprising three monocyclic rings together, where at least one of the three monocyclic rings is an aromatic or a heteroaromatic ring.

In some embodiments, Ar2 may be any of these options, that is, Ar2 is selected from (a) 5-membered monocyclic aromatic rings, (b) 6-membered monocyclic aromatic rings, (c) 9-membered fused bicyclic rings comprising one 5-membered and one 6-membered monocyclic ring fused together, where at least one of the two monocyclic rings, and optionally both of the monocyclic rings, is an aromatic ring, and (d) 10-membered fused bicyclic rings comprising two 6-membered monocyclic rings fused together, where at least one of the two monocyclic rings, and optionally both of the monocyclic rings, is an aromatic ring,

In compounds having Formulas (I), (IA), (IB), (IC), and/or (ID), optionally, Ar2 is a 5-membered monocyclic aromatic ring selected from the group consisting of thiophene, 1,2-thiazole, 1,3-thiazole, furan, 1,2-oxazole, 1,3-oxazole, 1H-pyrrole, 1H-pyrazole, oxadiazole, thiadiazole, 1,2,4-triazole, 1,2,3-triazole and 1H-imidazole.

In compounds having Formulas (I), (IA), (IB), (IC), and/or (ID), optionally, Ar2 is a 6-membered monocyclic aromatic ring selected from the group consisting of benzene, pyridine, pyridazine, pyrimidine and pyrazine.

In compounds having Formulas (I), (IA), (IB), (IC), and/or (ID), optionally, Ar2 is a 9-membered fused bicyclic aromatic ring system selected from the group consisting of benzofuran, 1,3-benzoxazole, furo[3,2-b]pyridine, furo[3,2-c]pyridine, furo[2,3-c]pyridine, furo[2,3-b]pyridine, indole, 1H-benzimidazole, 1H-pyrrolo[3,2-b]pyridine, 1H-pyrrolo[3,2-c]pyridine, 1H-pyrrolo[2,3-c]pyridine, 1H-pyrrolo[2,3-b]pyridine, benzothiophene, 1,3-benzothiazole, thienol[3,2-b]pyridine, thieno[3,2-c]pyridine, thieno[2,3-c]pyridine, benzoxadiazole, benzothiadiazole, benzisoxazole, benzotriazole and thieno[2,3-b]pyridine.

In compounds of Formula (I), (IA), (IB), (IC), and/or (ID), optionally, Ar2 is a 10-membered fused bicyclic aromatic ring system selected from the group consisting of naphthalene, quinoline, quinazoline, quinoxaline, 1,5-naphthyridine, 1,6-naphthyridine, 1,7-naphthyridine, 1,8-naphthyridine, isoquinoline, phthalazine, 2,6-naphthyridine and 2,7-naphthyridine.

As mentioned above, a compound of the present disclosure includes at least one Ar2 group, where the Ar2 group includes at least one aromatic ring and optionally includes one or more substituents on the aromatic ring, such as two or more substituents, such as three or more substituents, such as four or more substituents, where any of the one or more substituents may be the same or different. In some embodiments, Ar2 includes at least one, i.e., one or more, substituent on the aromatic ring, such as 1 to 5, or 1 to 4, or 1 to 3, or 1-2 substituents. In some embodiments, Ar2 includes exactly one substituent on the aromatic ring. In other embodiments, Ar2 includes exactly two substituents on the aromatic ring. In yet other embodiments, Ar2 includes exactly three substituents on the aromatic ring. In further embodiments, Ar2 includes exactly four substituents on the aromatic ring. In yet further embodiments, Ar2 includes two or more substituents on the aromatic ring.

In some embodiments, the one or more substituents on the ring atoms of Ar2 are selected from substituents optionally named “G”, where the substituents are selected from halogen, —CN, —NO₂, —SO₃R³, C₁-C₆ alkyl, C₁-C₆ haloalkyl, -E-O—R³, -E-(CH₂)ₐC(O)R³, -E-CO₂H, -E-CHO, -E-C(O)R³, -E-C(O)NH(OH), -E-C(O)NHR¹, -E-C(O)N(H)C(H)(R¹)(R^{1′}), -E-NR¹R^{1′}, -E-OR², -E-C(O)N(R¹)((CH₂)ₐP(O)(O)OH)₂, -E-C(O)N(R¹)((CH₂)ₐSO₃H), -E-C(O)NR¹R^{1′}, —C(O)-heterocycloalkyl (where the heterocycloalkyl is substituted or unsubstituted), —C(O)—N(H)—CH(COOH)((CH₂)ₐ-heteroaryl) (where heteroaryl is substituted or unsubstituted), wherein E is, at each occurrence, independently selected from a direct bond, and C₁-C₆ alkylene;

where R⁰ is, at each occurrence, independently selected from C₁-C₆ alkyl, C₁-C₆ haloalkyl, C₂-C₆ alkenyl, C₂-C₆ alkynyl, substituted or unsubstituted cycloalkyl, substituted or unsubstituted heterocyclyl, substituted or unsubstituted aryl, and substituted or unsubstituted heteroaryl;

R¹ and R^{1′} are, at each occurrence, independently selected from H, hydroxyl, C₁-C₆ alkyl, C₁-C₆ haloalkyl, C₁-C₂o heteroalkyl, C₁-C₁₀ heteroalkyl-NH₂, C₂-C₆ alkenyl, C₂-C₆ alkynyl, substituted or unsubstituted cycloalkyl, substituted or unsubstituted heterocyclyl (e.g., substituted with one or more R³ groups), substituted or unsubstituted aryl, substituted or unsubstituted arylalkyl, and substituted or unsubstituted heteroaryl, benzyl, —C(═NH)NH₂, —CH₂CO₂R⁰, —C(H)(CHₐ(COOH))₂, —CH₂C(O)NHCH₂CO₂H, —CH₂CH₂OH, —CH₂CH₂NHC(O)R³, —(CH₂)ₐC(O)NH(CH₂)ₐC(O)OR³, —(CH₂)ₐ—CN, spermine, —(CH₂)ₐ—NH₂, —C(O)R³, —(CH₂)ₐC(O)OR³, —(CH₂)ₐOR³, —C(H)((CH₂)ₐOH)_{a′}, —CHCH(OH)(CH₂)ₐOH, —(CH₂)ₐOH, C₁-C₆—C(O)OH, —(CH₂)ₐ-heterocycle which may be substituted (e.g., such as with an R³ group) or unsubstituted,

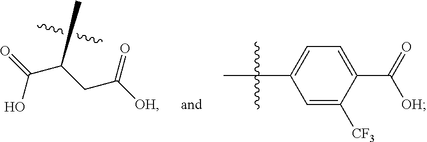

and

wherein R¹ and R^{1′} together form a substituted or unsubstituted heterocyclic ring, including, but not limited to, azetidine, pyrrolidine, piperidine, piperazine, morpholine,

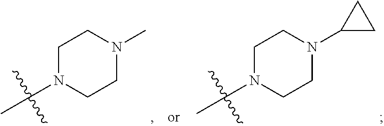

R² is, at each occurrence, independently selected from C₂-C₆ alkyl, C₁-C₆ haloalkyl, C₁-C₆ heteroalkyl, or substituted or unsubstituted cycloalkyl, substituted or unsubstituted heterocyclyl, substituted or unsubstituted aryl, substituted or unsubstituted heteroaryl, and substituted or unsubstituted haloalkoxy; and

R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine.

Non-limiting examples of substituents for Ar2 include, but are not limited to, H, —NH₂, —C(O)OH, —OH, —OCH₃, —C(O)NHOH, —C(O)NH₂, —(CH₂)₃C(O)OH, —CF₃, —C₁, —P(O)(OH)₂, —F, —C(O)N(H)CH₂CH₃, —C(O)N(H)CH₂C(O)OCH₃, —C(O)N(H)CH₂CF₂CF₂CF₃, —C(O)N(H)CH₂C(O)OH, —C(O)N(H)C(H)(C(O)OOH)CH₂C(O)OH, —C(O)N(H)CH₂C(O)N(H)CH₂C(O)OH, —SO₃H, —NO₂, —C(O)N(H)CH₂PO₃H₂, —C(O)N(H)CH₂3O₃H, —B(OH)₂, —C(OH)(CF₃)(CF₃), —C(O)-5-membered heterocycloalkyl-C(O)OH, —C(O)N(H)C(H)(C(O)OH)CH₂-imidazole, —S(O)(O)N(H)—CH₂COOH, —C(O)N(H)CH(C(O)OH)(CH₂CH₂C(O)OH), —C(O)N(H)CH₂CH₂SO₃H, —SO₂O⁻, —C(O)N(H)-imidazole-C(O)OH, and —C(O)N(CH₃)₂.

In some embodiments, the substitution on Ar2 includes amino (—NH₂). In some embodiments, the substitution on Ar2 includes alkoxy, e.g., C₁-C₆ alkoxy. For example, in one embodiment, the substitution on Ar2 includes methoxy. In some embodiments, the substitution on Ar2 includes carboxylic acid or alkylene-carboxylic acid. For example, in some embodiments, the substitution on Ar2 of PEM compounds of Formula (I) includes carboxylic acid. In some embodiments, the substitution on Ar2 includes carboxylic acid ester, or alkylene-carboxylic acid ester. For example, in one embodiment the substitution on Ar2 of PEM compounds of the Formula (I) includes —CH₂—CO₂—CH₃. In some embodiments, the substitution on Ar2 includes a haloalkyl group, e.g., a C₁-C₆ haloalkyl group. For example, in one embodiment, the substitution on Ar2 of a PEM compounds of Formula (I) includes trifluoromethyl. In some embodiments, the substitution on Ar2 includes hydroxyl or hydroxyl-substituted alkyl, e.g., hydroxyl-substituted C₁-C₆ alkyl. For example, in one embodiment, the substitution on Ar2 of a compound of Formula (I) includes hydroxyl (—OH).

In some embodiments, the substitution on Ar2 includes one group selected from carboxylic acid and alkylene-carboxylic acid, e.g., C₁-C₆ alkylene-carboxylic acid, and another group selected from hydroxyl and hydroxyl-substituted alkyl, e.g., C₁-C₆ alkyl substituted with one hydroxyl. For example, in one embodiment, the substitution on Ar2 is, or includes one carboxylic acid and one hydroxyl.

In some embodiments, the substitution on Ar2 includes one group selected from carboxylic acid and alkylene-carboxylic acid, e.g., C₁-C₆ alkylene-carboxylic acid, and one group selected from haloalkyl, e.g., C₁-C₆ haloalkyl. For example, in one embodiment, the substitution on Ar2 is, or includes one carboxylic acid group and one trifluoromethyl group.

In some embodiments, the substitution on Ar2 includes one group selected from hydroxyl and hydroxyl-substituted alkyl, e.g., C₁-C₆ alkyl substituted with one hydroxyl, and another group selected from haloalkyl, e.g., C₁-C₆ haloalkyl. For example, in one embodiment, the substitution on Ar2 is, or includes one hydroxyl group and one trifluoromethyl group.

In some embodiments, the substitution on the Ar2 ring of Formula (I) includes at least of one of a) carboxylic acid and alkylene-carboxylic acid, e.g., C₁-C₆ alkylene-carboxylic acid; b) hydroxyl and hydroxyl-substituted alkyl, e.g., C₁-C₆ alkyl substituted with one hydroxyl; and c) haloalkyl, e.g., C₁-C₆ haloalkyl. For example, at least one of carboxylic acid, hydroxyl and trifluoromethyl.

In some embodiments, the substitution on the Ar2 ring of Formula (I) includes at least two of a) carboxylic acid and alkylene-carboxylic acid, e.g., C₁-C₆ alkylene-carboxylic acid; b) hydroxyl and hydroxyl-substituted alkyl, e.g., C₁-C₆ alkyl substituted with one hydroxyl; and c) haloalkyl, e.g., C₁-C₆ haloalkyl. For example, at least two of carboxylic acid, hydroxyl and trifluoromethyl.

In some embodiments, the substitution on the Ar2 ring of Formula (I) includes all three of a) carboxylic acid and alkylene-carboxylic acid, e.g., C₁-C₆ alkylene-carboxylic acid; b) hydroxyl and hydroxyl-substituted alkyl, e.g., C₁-C₆ alkyl substituted with one hydroxyl; and c) haloalkyl, e.g., C₁-C₆ haloalkyl. That is, Ar2 may be substituted with carboxylic acid, hydroxyl and trifluoromethyl.

In some embodiments, the substitution on the Ar2 ring of Formula (I) includes at least one —SO₃H moiety, or the salt or hydrate thereof. In some embodiments, the substitution on the Ar2 ring of Formula (I) includes at least two —SO₃H moieties (at any positions relative to each other on the Ar2 ring), or the salt or hydrate thereof.

In some embodiments, the Ar2 ring of Formula (I) includes at least two substituents, where a first substituent of the at least two substituents is a —SO₃H moiety or a —SO₃⁻ moiety; and wherein a second substituent of the at least two substituents is selected from any of the “G” substituents described herein. In some embodiments, the Ar2 ring of Formula (I) includes at least two substituents, where a first substituent of the at least two substituents is a —SO₃H moiety (or the salt or hydrate thereof); and wherein a second substituent of the at least two substituents is selected from —NO₂, —CF₃, —F, —Cl, —I, methyl, ethyl, propyl, and butyl. In some embodiments, the Ar2 ring of Formula (I) includes at least three substituents, where a first substituent of the at least two substituents is a —SO₃H moiety or a —SO₃⁻ moiety; and wherein a second and a third substituent of the at least three substituents is selected from —NO₂, —CF₃, —F, —Cl, —I, methyl, ethyl, propyl, and butyl.

In some embodiments, the Ar2 ring of Formula (I) includes at least three substituents, where a first substituent of the at least two substituents is a —SO₃H moiety or a —SO₃⁻ moiety; and wherein a second substituent of the at least three substituents is substituents is a —SO₃H moiety; and wherein a third substituent of the at least three substituents is selected from —NO₂, —CF₃, —F, methyl, ethyl, propyl, and butyl.

In some embodiments, the substitution on the Ar2 ring of Formula (I) includes at least one —S(O)(O)N(H)CH₂C(O)OH moiety, at least one —C(O)N(H)CH₂CH₂SO₃H moiety, or at least one —C(O)N(H)CH₂PO₃H₂. In other embodiments, an Ar2 moiety is substituted with at least two substituents, where a first substituent of the at least two substituents is one of —S(O)(O)N(H)CH₂C(O)OH, —C(O)N(H)CH₂CH₂SO₃H, or —C(O)N(H)CH₂PO₃H₂.

In some embodiments, the substitution on the Ar2 ring of Formula (I) includes at least an —SO₂O⁻ moiety. In other embodiments, the substitution on the Ar2 ring of Formula (I) includes at least an —SO₂O⁻ moiety and at least one other moiety, including any of those moieties described herein for “G.”

For example, in some embodiments, the Ar2 group is a substituted phenyl group selected from:

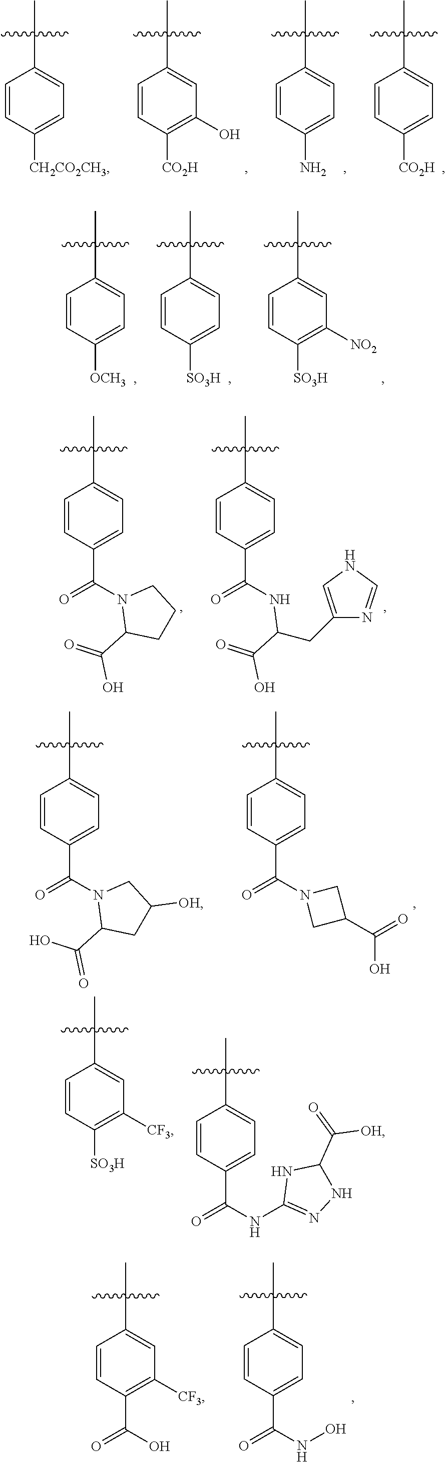

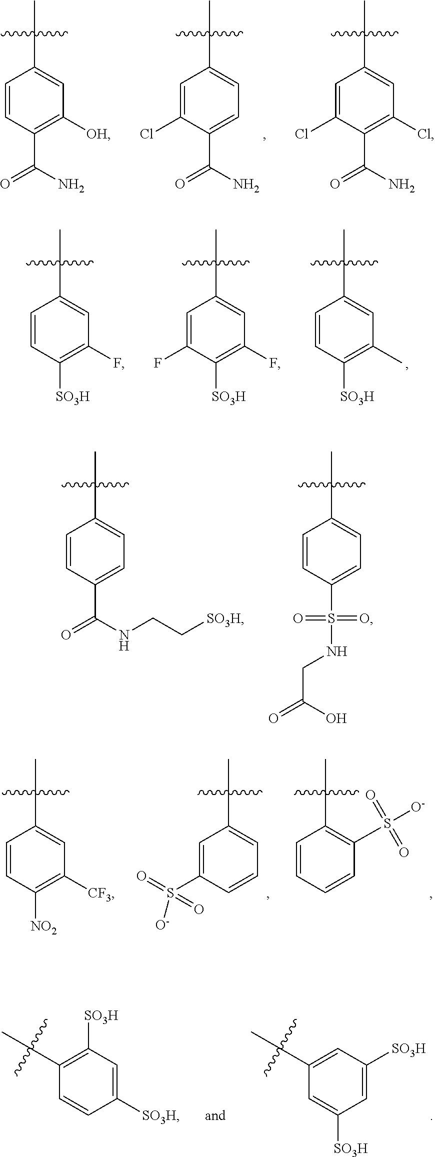

In some embodiments, the Ar2 group is a substituted phenyl group, wherein the substituent of the phenyl group is aryl further substituted with G², G³, G⁴ and G⁵, and in a more specific embodiment the substituent of the phenyl group is phenyl further substituted with G², G³, G⁴ and G⁵, such as phenyl further substituted with G² or with G² and G³. For instance, the Ar2 group may be substituted with an —SO₃H moiety or —SO₃⁻, and may be further substituted with G², G³, G⁴ and G⁵, such as phenyl further substituted with G² or with G² and G³. By way of another example, the Ar2 group may be substituted with an —C(O)OH moiety, and may be further substituted with G², G³, G⁴ and G⁵, such as phenyl further substituted with G² or with G² and G³. By way of yet another example, the Ar2 group may be substituted with one of a —S(O)(O)N(H)CH₂C(O)OH moiety, a —C(O)N(H)CH₂CH₂SO₃H moiety, or a —C(O)N(H)CH₂PO₃H₂, and may be further substituted with G², G³, G⁴ and G⁵, such as phenyl further substituted with G² or with G² and G³. By way of yet another example, the Ar2 group may be substituted with an —SO₂O⁻ moiety, and may be further substituted with G², G³, G⁴ and G⁵, such as phenyl further substituted with G² or with G² and G³.

As mentioned, in one embodiment, the PEM compounds of the present disclosure may have hydroxyl and carboxylic acid substitution on Ar₂. These two groups may be located at various positions on the Ar₂ ring.

For instance, in some embodiments, the PEM compounds are described by the formula:

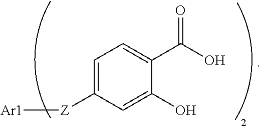

In some embodiments, Z is selected from imidazole, pyrazole, pyrimidine, pyridazine, pyrazine, pyrrole, thiophene, triazole (e.g., 1,2,3-triazole; 1,2,4-triazole, thiazole, and isoxazole. In some embodiments, Z is not a triazole. In some embodiments, in some embodiments, Z is not 1,2,3-triazole or 1,2,4-triazole. In some embodiments, Ar1 is selected from pyridine, bipyridine, phenyl, and carbazole. In some embodiments, Ar1 may be substituted with —C(O)—CH₃, —C(O)—NH₂, —C(O)O—CH₂CH₃, —CF₃, —C(O)O-t-butyl, —C(O)—N(CH₃)₂, —C(O)—N(H)CH₃, —C(O)—N(H)CH₂CH₃, —C(O)—N(H)(cycloalkyl), —C(O)—N(H)(CH₂)₄, —C(O)—N(CH₂CH₃)₂, —C(O)—N(H)(t-butyl), —C(O)—N(H)(C6-cycloalkyl), —C(O)—N(H)(CH₂)₃, —C(O)—N(H)(C₅-cycloalkyl), —C(O)—N(H)(C₄-cycloalkyl), —C(O)—N(H)(phenyl), —C(O)—N(H)(CH₂CH₂)N(H)C(O)—CH₃, —C(O)—N(H)C(NH)(NH₂), —C(O)—N(CH₂CH₂CH₃)₂, —C(O)—N—(C(H)(CH₃)(CH₂CH₃))₂, —C(O)—N(CH₂CH₂CH₂CH₃)₂, —C(O)—N(H)(benzyl), —C(O)—N(H)—CH₂CH₂OH, —C(O)—S—CH₂CH₃, —C(O)N(CH₂)₂, —N(H)S(O)(O)-phenyl, —C(O)N(H)CH₂CH₂CF₃, —C(O)N(H)CH₃, —C(O)OCH₂CH₃, —C(O)—N(H)—CH₂CH₂O—CH₂CH₂O—CH₃, —C(O)—N(H)—(CH₂CH₂O)₈—CH₃, —C(O)N(H)—CH₂—CH₂—CH₂—N(H)—CH₂—CH₂—CH₂—N(H)—CH₂—CH₂—CH₂—NH₂, —C(O)N(H)—CH₂—CH₂—NH₂, —C(O)N(H)C(O)CH₃, —C(O)N(H)—CH₂—CH₃, —S(O)(O)N(H)—CH₂—CH₃, —S(O)(O)N—(CH₂—CH₃)₂, —S(O)(O)N(H)—(C₃-cycloalkyl), —S(O)(O)N(H)—(CH₂CH₂)OH, —S(O)(O)OH, —C(O)N(H)C(H)(CH₂OH)₂, —C(O)N(CH₂CH₂OH)₂, —C(O)N(H)CH₂CH₂═CH₂, —C(O)CH₂N(CH₃)(CH₂)(COOH), —S(O)(O)(CH₂CH₃), —S(O)(O)N(H)CH₂—CH(OH)(CH₂OH), —S(O)(O)—NH₂, or S(O)(O)N(H)CH₂—C(O)(O-t-butyl).

In one particular embodiment, Z is a triazole, and the compound of Formula (I) has the structure:

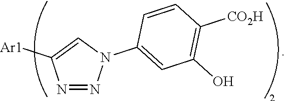

In another embodiment, the PEM compounds of the present disclosure have hydroxyl and carboxylic acid substitution on Ar₂ as provided in the formula:

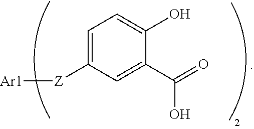

In some embodiments, Z is selected from imidazole, pyrazole, pyrimidine, pyridazine, pyrazine, pyrrole, thiophene, triazole (e.g., 1,2,3-triazole; 1,2,4-triazole, thiazole, and isoxazole. In some embodiments, Z is not a triazole. In some embodiments, in some embodiments, Z is not 1,2,3-triazole or 1,2,4-triazole. In some embodiments, Ar1 is selected from pyridine, bipyridine, phenyl, and carbazole. In some embodiments, Ar1 may be substituted with —C(O)—CH₃, —C(O)—NH₂, —C(O)O—CH₂CH₃, —CF₃, —C(O)O-t-butyl, —C(O)—N(CH₃)₂, —C(O)—N(H)CH₃, —C(O)—N(H)CH₂CH₃, —C(O)—N(H)(cycloalkyl), —C(O)—N(H)(CH₂)₄, —C(O)—N(CH₂CH₃)₂, —C(O)—N(H)(t-butyl), —C(O)—N(H)(C6-cycloalkyl), —C(O)—N(H)(CH₂)₃, —C(O)—N(H)(C5-cycloalkyl), —C(O)—N(H)(C4-cycloalkyl), —C(O)—N(H)(phenyl), —C(O)—N(H)(CH₂CH₂)N(H)C(O)—CH₃, —C(O)—N(H)C(NH)(NH₂), —C(O)—N(CH₂CH₂CH₃)₂, —C(O)—N—(C(H)(CH₃)(CH₂CH₃))₂, —C(O)—N(CH₂CH₂CH₂CH₃)₂, —C(O)—N(H)(benzyl), —C(O)—N(H)—CH₂CH₂OH, —C(O)—S—CH₂CH₃, —C(O)N(CH₂)₂, —N(H)S(O)(O)-phenyl, —C(O)N(H)CH₂CH₂CF₃, —C(O)N(H)CH₃, —C(O)OCH₂CH₃, —C(O)—N(H)—CH₂CH₂O—CH₂CH₂O—CH₃, —C(O)—N(H)—(CH₂CH₂O)₈—CH₃, —C(O)N(H)—CH₂—CH₂—CH₂—N(H)—CH₂—CH₂—CH₂—N(H)—CH₂—CH₂—CH₂—NH₂, —C(O)N(H)—CH₂—CH₂—NH₂, —C(O)N(H)C(O)CH₃, —C(O)N(H)—CH₂—CH₃, —S(O)(O)N(H)—CH₂—CH₃, —S(O)(O)N—(CH₂—CH₃)₂, —S(O)(O)N(H)—(C3-cycloalkyl), —S(O)(O)N(H)—(CH₂CH₂)OH, —S(O)(O)OH, —C(O)N(H)C(H)(CH₂OH)₂, —C(O)N(CH₂CH₂OH)₂, —C(O)N(H)CH₂CH₂═CH₂, —C(O)CH₂N(CH₃)(CH₂)(COOH), —S(O)(O)(CH₂CH₃), —S(O)(O)N(H)CH₂—CH(OH)(CH₂OH), —S(O)(O)—NH₂, or S(O)(O)N(H)CH₂—C(O)(O-t-butyl).

In one particular embodiment, Z is a triazole, and the compound of Formula (I) has the structure:

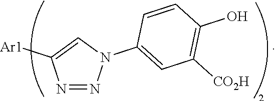

In yet another embodiment, the PEM compounds of the present disclosure have hydroxyl and carboxylic acid substitution on Ar₂ as shown in the formula:

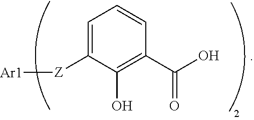

In some embodiments, Z is selected from imidazole, pyrazole, pyrimidine, pyridazine, pyrazine, pyrrole, thiophene, triazole (e.g., 1,2,3-triazole; 1,2,4-triazole, thiazole, and isoxazole. In some embodiments, Z is not a triazole. In some embodiments, in some embodiments, Z is not 1,2,3-triazole or 1,2,4-triazole. In some embodiments, Ar1 is selected from pyridine, bipyridine, phenyl, and carbazole. In some embodiments, Ar1 may be substituted with —C(O)—CH₃, —C(O)—NH₂, —C(O)O—CH₂CH₃, —CF₃, —C(O)O-t-butyl, —C(O)—N(CH₃)₂, —C(O)—N(H)CH₃, —C(O)—N(H)CH₂CH₃, —C(O)—N(H)(cycloalkyl), —C(O)—N(H)(CH₂)₄, —C(O)—N(CH₂CH₃)₂, —C(O)—N(H)(t-butyl), —C(O)—N(H)(C6-cycloalkyl), —C(O)—N(H)(CH₂)₃, —C(O)—N(H)(C5-cycloalkyl), —C(O)—N(H)(C4-cycloalkyl), —C(O)—N(H)(phenyl), —C(O)—N(H)(CH₂CH₂)N(H)C(O)—CH₃, —C(O)—N(H)C(NH)(NH₂), —C(O)—N(CH₂CH₂CH₃)₂, —C(O)—N—(C(H)(CH₃)(CH₂CH₃))₂, —C(O)—N(CH₂CH₂CH₂CH₃)₂, —C(O)—N(H)(benzyl), —C(O)—N(H)—CH₂CH₂OH, —C(O)—S—CH₂CH₃, —C(O)N(CH₂)₂, —N(H)S(O)(O)-phenyl, —C(O)N(H)CH₂CH₂CF₃, —C(O)N(H)CH₃, —C(O)OCH₂CH₃, —C(O)—N(H)—CH₂CH₂O—CH₂CH₂O—CH₃, —C(O)—N(H)—(CH₂CH₂O)₈—CH₃, —C(O)N(H)—CH₂—CH₂—CH₂—N(H)—CH₂—CH₂—CH₂—N(H)—CH₂—CH₂—CH₂—NH₂, —C(O)N(H)—CH₂—CH₂—NH₂, —C(O)N(H)C(O)CH₃, —C(O)N(H)—CH₂—CH₃, —S(O)(O)N(H)—CH₂—CH₃, —S(O)(O)N—(CH₂—CH₃)₂, —S(O)(O)N(H)—(C3-cycloalkyl), —S(O)(O)N(H)—(CH₂CH₂)OH, —S(O)(O)OH, —C(O)N(H)C(H)(CH₂OH)₂, —C(O)N(CH₂CH₂OH)₂, —C(O)N(H)CH₂CH₂═CH₂, —C(O)CH₂N(CH₃)(CH₂)(COOH), —S(O)(O)(CH₂CH₃), —S(O)(O)N(H)CH₂—CH(OH)(CH₂OH), —S(O)(O)—NH₂, or S(O)(O)N(H)CH₂—C(O)(O-t-butyl).

In one particular embodiment, Z is a triazole, and the compound of Formula (I) has the structure:

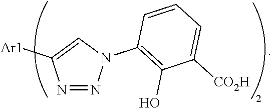

In one embodiment, the PEM compounds of the present disclosure have at least hydroxyl and carboxylic substitution on Ar₂ and may have other substitution on Ar₂. For instance, Ar₂ may be substituted with hydroxyl, carboxylic acid, and alkyl, e.g., C₁-C₆ alkyl, to provide, e.g., a compound of the formula:

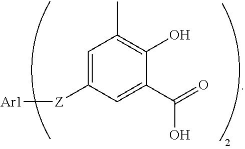

In some embodiments, Z is selected from imidazole, pyrazole, pyrimidine, pyridazine, pyrazine, pyrrole, thiophene, triazole (e.g., 1,2,3-triazole; 1,2,4-triazole, thiazole, and isoxazole. In some embodiments, Z is not a triazole. In some embodiments, in some embodiments, Z is not 1,2,3-triazole or 1,2,4-triazole. In some embodiments, Ar1 is selected from pyridine, bipyridine, phenyl, and carbazole. In some embodiments, Ar1 may be substituted with —C(O)—CH₃, —C(O)—NH₂, —C(O)O—CH₂CH₃, —CF₃, —C(O)O-t-butyl, —C(O)—N(CH₃)₂, —C(O)—N(H)CH₃, —C(O)—N(H)CH₂CH₃, —C(O)—N(H)(cycloalkyl), —C(O)—N(H)(CH₂)₄, —C(O)—N(CH₂CH₃)₂, —C(O)—N(H)(t-butyl), —C(O)—N(H)(C₆-cycloalkyl), —C(O)—N(H)(CH₂)₃, —C(O)—N(H)(C₅-cycloalkyl), —C(O)—N(H)(C₄-cycloalkyl), —C(O)—N(H)(phenyl), —C(O)—N(H)(CH₂CH₂)N(H)C(O)—CH₃, —C(O)—N(H)C(NH)(NH₂), —C(O)—N(CH₂CH₂CH₃)₂, —C(O)—N—(C(H)(CH₃)(CH₂CH₃))₂, —C(O)—N(CH₂CH₂CH₂CH₃)₂, —C(O)—N(H)(benzyl), —C(O)—N(H)—CH₂CH₂OH, —C(O)—S—CH₂CH₃, —C(O)N(CH₂)₂, —N(H)S(O)(O)-phenyl, —C(O)N(H)CH₂CH₂CF₃, —C(O)N(H)CH₃, —C(O)OCH₂CH₃, —C(O)—N(H)—CH₂CH₂O—CH₂CH₂O—CH₃, —C(O)—N(H)—(CH₂CH₂O)₈—CH₃, —C(O)N(H)—CH₂—CH₂—CH₂—N(H)—CH₂—CH₂—CH₂—N(H)—CH₂—CH₂—CH₂—NH₂, —C(O)N(H)—CH₂—CH₂—NH₂, —C(O)N(H)C(O)CH₃, —C(O)N(H)—CH₂—CH₃, —S(O)(O)N(H)—CH₂—CH₃, —S(O)(O)N—(CH₂—CH₃)₂, —S(O)(O)N(H)—(C3-cycloalkyl), —S(O)(O)N(H)—(CH₂CH₂)OH, —S(O)(O)OH, —C(O)N(H)C(H)(CH₂OH)₂, —C(O)N(CH₂CH₂OH)₂, —C(O)N(H)CH₂CH₂═CH₂, —C(O)CH₂N(CH₃)(CH₂)(COOH), —S(O)(O)(CH₂CH₃), —S(O)(O)N(H)CH₂—CH(OH)(CH₂OH), —S(O)(O)—NH₂, or S(O)(O)N(H)CH₂—C(O)(O-t-butyl).

In one particular embodiment, Z is a triazole, and the compound of Formula (I) has the structure:

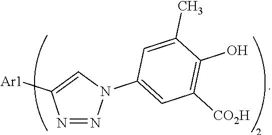

As mentioned previously, in one embodiment, the PEM compounds of the present disclosure may have haloalkyl and carboxylic acid substitution on Ar2 rather than hydroxyl and carboxylic acid as illustrated in the structures above.

In one particular embodiment, the PEM compounds of the present disclosure may be described by the formula:

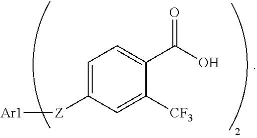

In some embodiments, Z is selected from imidazole, pyrazole, pyrimidine, pyridazine, pyrazine, pyrrole, thiophene, triazole (e.g., 1,2,3-triazole; 1,2,4-triazole, thiazole, and isoxazole. In some embodiments, Z is not a triazole. In some embodiments, in some embodiments, Z is not 1,2,3-triazole or 1,2,4-triazole. In some embodiments, Ar1 is selected from pyridine, bipyridine, phenyl, and carbazole. In some embodiments, Ar1 may be substituted with —C(O)—CH₃, —C(O)—NH₂, —C(O)O—CH₂CH₃, —CF₃, —C(O)O-t-butyl, —C(O)—N(CH₃)₂, —C(O)—N(H)CH₃, —C(O)—N(H)CH₂CH₃, —C(O)—N(H)(cycloalkyl), —C(O)—N(H)(CH₂)₄, —C(O)—N(CH₂CH₃)₂, —C(O)—N(H)(t-butyl), —C(O)—N(H)(C6-cycloalkyl), —C(O)—N(H)(CH₂)₃, —C(O)—N(H)(C5-cycloalkyl), —C(O)—N(H)(C4-cycloalkyl), —C(O)—N(H)(phenyl), —C(O)—N(H)(CH₂CH₂)N(H)C(O)—CH₃, —C(O)—N(H)C(NH)(NH₂), —C(O)—N(CH₂CH₂CH₃)₂, —C(O)—N—(C(H)(CH₃)(CH₂CH₃))₂, —C(O)—N(CH₂CH₂CH₂CH₃)₂, —C(O)—N(H)(benzyl), —C(O)—N(H)—CH₂CH₂OH, —C(O)—S—CH₂CH₃, —C(O)N(CH₂)₂, —N(H)S(O)(O)-phenyl, —C(O)N(H)CH₂CH₂CF₃, —C(O)N(H)CH₃, —C(O)OCH₂CH₃, —C(O)—N(H)—CH₂CH₂O—CH₂CH₂O—CH₃, —C(O)—N(H)—(CH₂CH₂O)₈—CH₃, —C(O)N(H)—CH₂—CH₂—CH₂—N(H)—CH₂—CH₂—CH₂—N(H)—CH₂—CH₂—CH₂—NH₂, —C(O)N(H)—CH₂—CH₂—NH₂, —C(O)N(H)C(O)CH₃, —C(O)N(H)—CH₂—CH₃, —S(O)(O)N(H)—CH₂—CH₃, —S(O)(O)N—(CH₂—CH₃)₂, —S(O)(O)N(H)—(C3-cycloalkyl), —S(O)(O)N(H)—(CH₂CH₂)OH, —S(O)(O)OH, —C(O)N(H)C(H)(CH₂OH)₂, —C(O)N(CH₂CH₂OH)₂, —C(O)N(H)CH₂CH₂═CH₂, —C(O)CH₂N(CH₃)(CH₂)(COOH), —S(O)(O)(CH₂CH₃), —S(O)(O)N(H)CH₂—CH(OH)(CH₂OH), —S(O)(O)—NH₂, or S(O)(O)N(H)CH₂—C(O)(O-t-butyl).

In one particular embodiment, Z is a triazole, and the compound of Formula (I) has the structure:

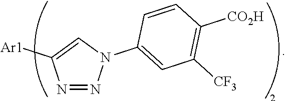

In some embodiments, and as noted herein, the PEM compounds of the present disclosure include one or more —SO₃H moieties or —SO₂O⁻ moieties, and optionally one or more additional substituents on Ar2 (such as any one of G², G³, G⁴, or G⁵ as described herein. Examples of such PEM compounds include, but are not limited to, those recited below:

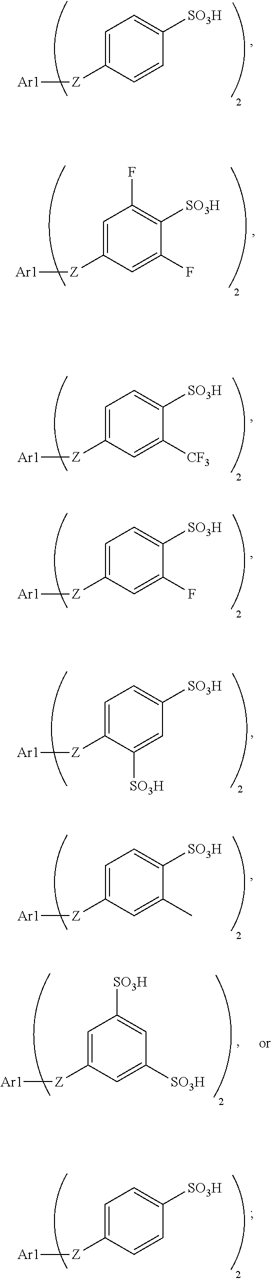

or the salt or hydrate thereto.

In some embodiments, Z is selected from imidazole, pyrazole, pyrimidine, pyridazine, pyrazine, pyrrole, thiophene, triazole (e.g., 1,2,3-triazole; 1,2,4-triazole, thiazole, and isoxazole. In some embodiments, Z is not a triazole. In some embodiments, in some embodiments, Z is not 1,2,3-triazole or 1,2,4-triazole. In some embodiments, Ar1 is selected from pyridine, bipyridine, phenyl, and carbazole. In some embodiments, the group Ar1 may be substituted (such as with any of the substituents noted herein), or unsubstituted. For instance, Ar1 may be substituted with —C(O)—CH₃, —C(O)—NH₂, —C(O)O—CH₂CH₃, —CF₃, —C(O)O-t-butyl, —C(O)—N(CH₃)₂, —C(O)—N(H)CH₃, —C(O)—N(H)CH₂CH₃, —C(O)—N(H)(cycloalkyl), —C(O)—N(H)(CH₂)₄, —C(O)—N(CH₂CH₃)₂, —C(O)—N(H)(t-butyl), —C(O)—N(H)(C6-cycloalkyl), —C(O)—N(H)(CH₂)₃, —C(O)—N(H)(C5-cycloalkyl), —C(O)—N(H)(C4-cycloalkyl), —C(O)—N(H)(phenyl), —C(O)—N(H)(CH₂CH₂)N(H)C(O)—CH₃, —C(O)—N(H)C(NH)(NH₂), —C(O)—N(CH₂CH₂CH₃)₂, —C(O)—N—(C(H)(CH₃)(CH₂CH₃))₂, —C(O)—N(CH₂CH₂CH₂CH₃)₂, —C(O)—N(H)(benzyl), —C(O)—N(H)—CH₂CH₂OH, —C(O)—S—CH₂CH₃, —C(O)N(CH₂)₂, —N(H)S(O)(O)-phenyl, —C(O)N(H)CH₂CH₂CF₃, —C(O)N(H)CH₃, —C(O)OCH₂CH₃, —C(O)—N(H)—CH₂CH₂O—CH₂CH₂O—CH₃, —C(O)—N(H)—(CH₂CH₂O)₈—CH₃, —C(O)N(H)—CH₂—CH₂—CH₂—N(H)—CH₂—CH₂—CH₂—N(H)—CH₂—CH₂—CH₂—NH₂, —C(O)N(H)—CH₂—CH₂—NH₂, —C(O)N(H)C(O)CH₃, —C(O)N(H)—CH₂—CH₃, —S(O)(O)N(H)—CH₂—CH₃, —S(O)(O)N—(CH₂—CH₃)₂, —S(O)(O)N(H)—(C3-cycloalkyl), —S(O)(O)N(H)—(CH₂CH₂)OH, —S(O)(O)OH, —C(O)N(H)C(H)(CH₂OH)₂, —C(O)N(CH₂CH₂OH)₂, —C(O)N(H)CH₂CH₂═CH₂, —C(O)CH₂N(CH₃)(CH₂)(COOH), —S(O)(O)(CH₂CH₃), —S(O)(O)N(H)CH₂—CH(OH)(CH₂OH), —S(O)(O)—NH₂, or S(O)(O)N(H)CH₂—C(O)(O-t-butyl).

In some embodiments, Z is a triazole, and the above-recited compounds of Formula (I) have the structure:

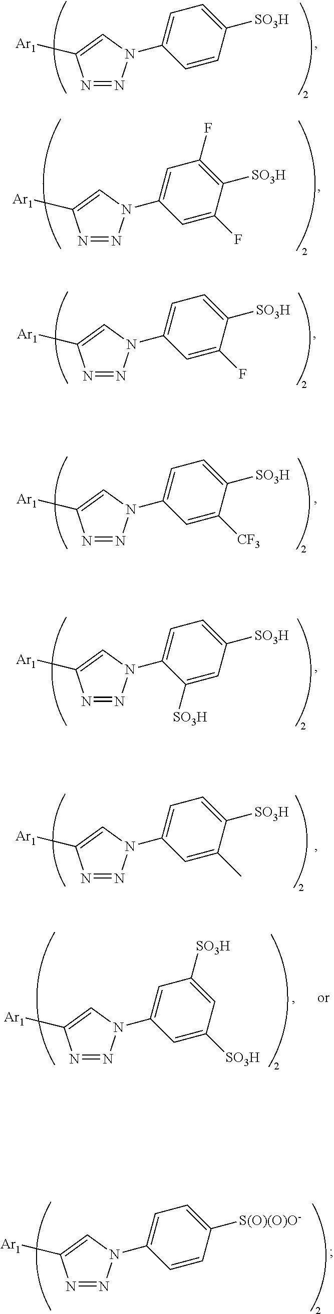

or the salt or hydrate thereof.

In some embodiments, Ar1 may be substituted with —C(O)—CH₃, —C(O)—NH₂, —C(O)O—CH₂CH₃, —CF₃, —C(O)O-t-butyl, —C(O)—N(CH₃)₂, —C(O)—N(H)CH₃, —C(O)—N(H)CH₂CH₃, —C(O)—N(H)(cycloalkyl), —C(O)—N(H)(CH₂)₄, —C(O)—N(CH₂CH₃)₂, —C(O)—N(H)(t-butyl), —C(O)—N(H)(C6-cycloalkyl), —C(O)—N(H)(CH₂)₃, —C(O)—N(H)(C5-cycloalkyl), —C(O)—N(H)(C4-cycloalkyl), —C(O)—N(H)(phenyl), —C(O)—N(H)(CH₂CH₂)N(H)C(O)—CH₃, —C(O)—N(H)C(NH)(NH₂), —C(O)—N(CH₂CH₂CH₃)₂, —C(O)—N—(C(H)(CH₃)(CH₂CH₃))₂, —C(O)—N(CH₂CH₂CH₂CH₃)₂, —C(O)—N(H)(benzyl), —C(O)—N(H)—CH₂CH₂OH, —C(O)—S—CH₂CH₃, —C(O)N(CH₂)₂, —N(H)S(O)(O)-phenyl, —C(O)N(H)CH₂CH₂CF₃, —C(O)N(H)CH₃, —C(O)OCH₂CH₃, —C(O)—N(H)—CH₂CH₂O—CH₂CH₂O—CH₃, —C(O)—N(H)—(CH₂CH₂O)₈—CH₃, —C(O)N(H)—CH₂—CH₂—CH₂—N(H)—CH₂—CH₂—CH₂—N(H)—CH₂—CH₂—CH₂—NH₂, —C(O)N(H)—CH₂—CH₂—NH₂, —C(O)N(H)C(O)CH₃, —C(O)N(H)—CH₂—CH₃, —S(O)(O)N(H)—CH₂—CH₃, —S(O)(O)N—(CH₂—CH₃)₂, —S(O)(O)N(H)—(C3-cycloalkyl), —S(O)(O)N(H)—(CH₂CH₂)OH, —S(O)(O)OH, —C(O)N(H)C(H)(CH₂OH)₂, —C(O)N(CH₂CH₂OH)₂, —C(O)N(H)CH₂CH₂═CH₂, —C(O)CH₂N(CH₃)(CH₂)(COOH), —S(O)(O)(CH₂CH₃), —S(O)(O)N(H)CH₂—CH(OH)(CH₂OH), —S(O)(O)—NH₂, or S(O)(O)N(H)CH₂—C(O)(O-t-butyl).

Yet other examples of PEM compounds in accordance with the present disclosure that include an Ar2 moiety substituted with —SO₃H (or the salt thereof) include, but are not limited to, those compounds recited below:

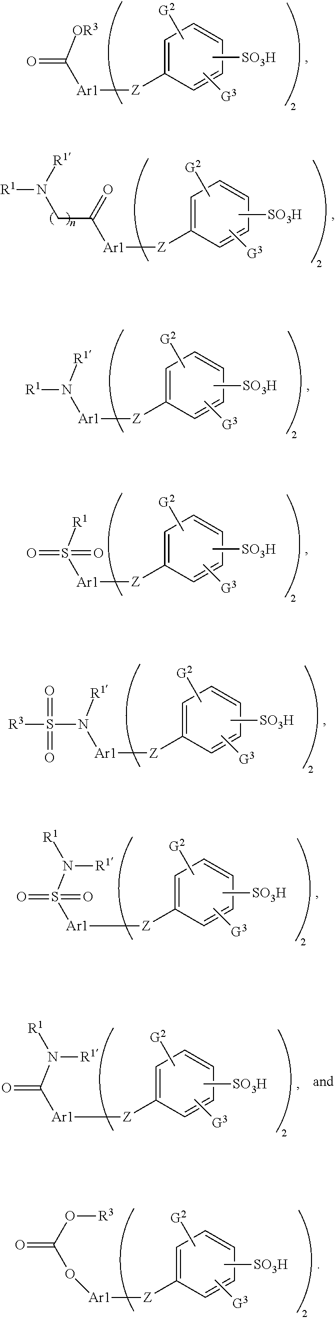

In these embodiments, the —SO₃H group (or the salt thereof, namely —SO₃⁻) may be provided at any ring position, such as para to the group “Z,” meta to the group “Z,” or ortho to the group “Z.”.

In some embodiments, Z is selected from imidazole, pyrazole, pyrimidine, pyridazine, pyrazine, pyrrole, thiophene, triazole (e.g., 1,2,3-triazole; 1,2,4-triazole, thiazole, tetrazole, and isoxazole. In some embodiments, Z is not a triazole. In some embodiments, in some embodiments, Z is not 1,2,3-triazole or 1,2,4-triazole.

In some embodiments, Ar1 is selected from pyridine, bipyridine, phenyl, and carbazole.

Yet further examples of PEM compounds in accordance with the present disclosure that include an Ar2 moiety substituted with —SO₃H (or the salt thereof) include, but are not limited to, those compounds recited below:

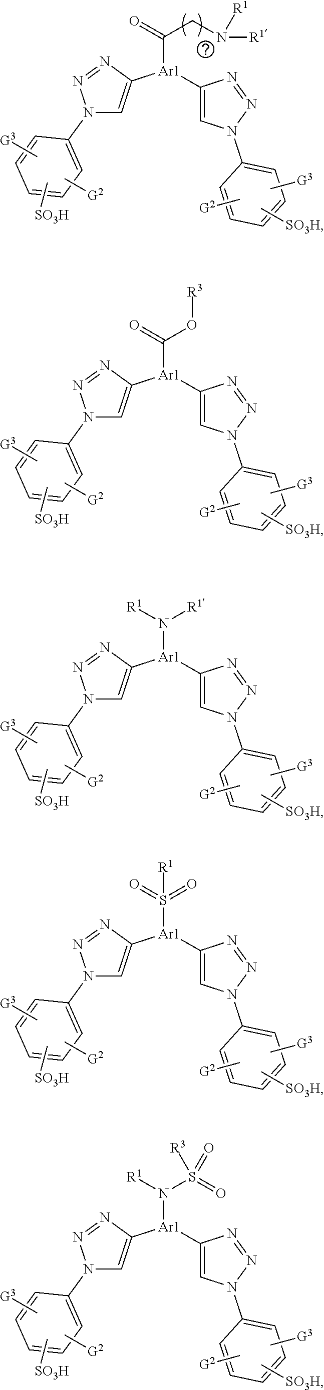

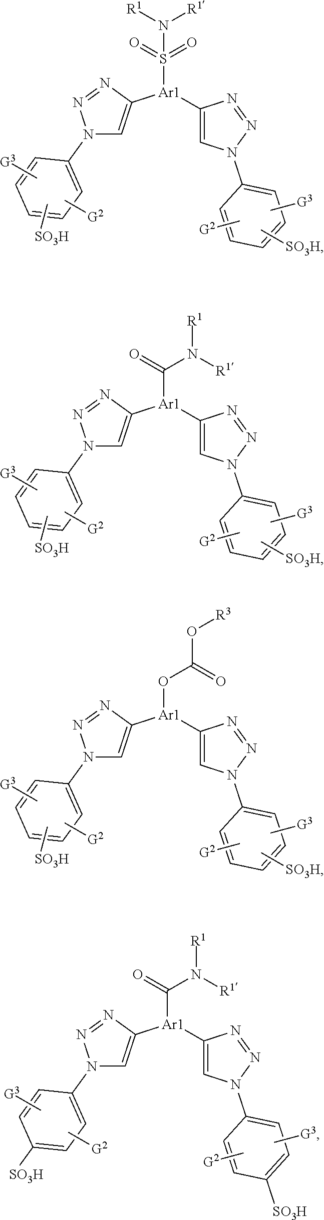

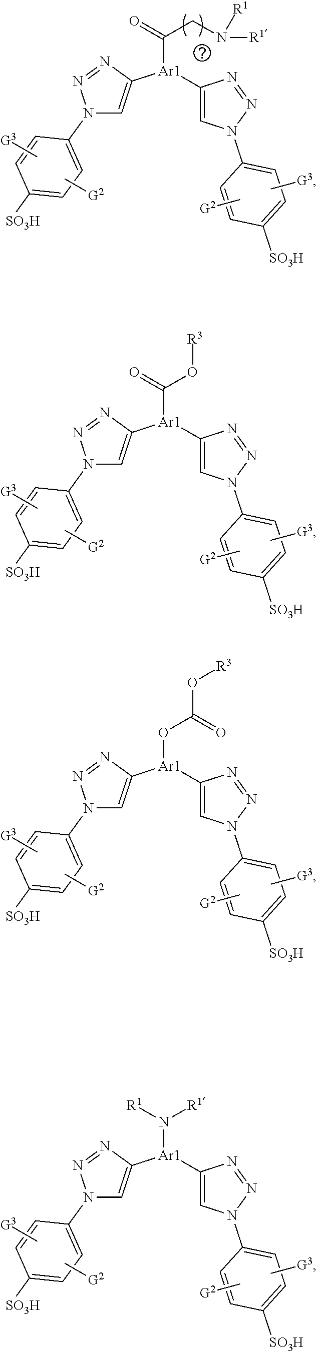

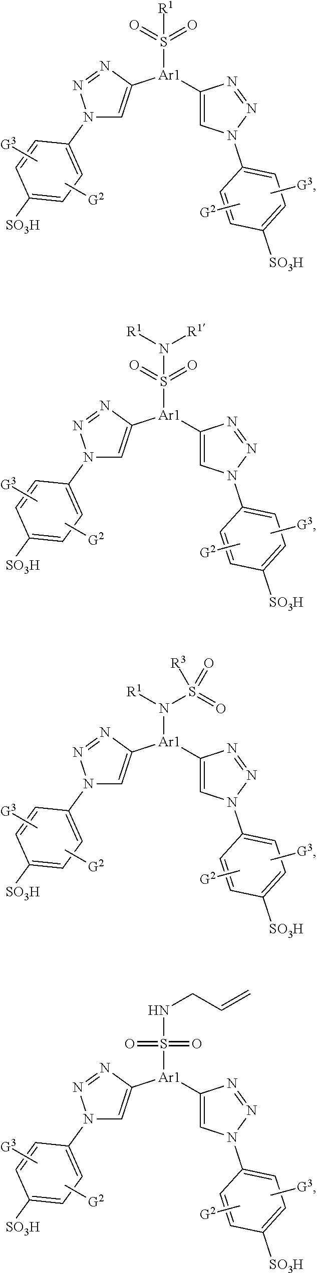

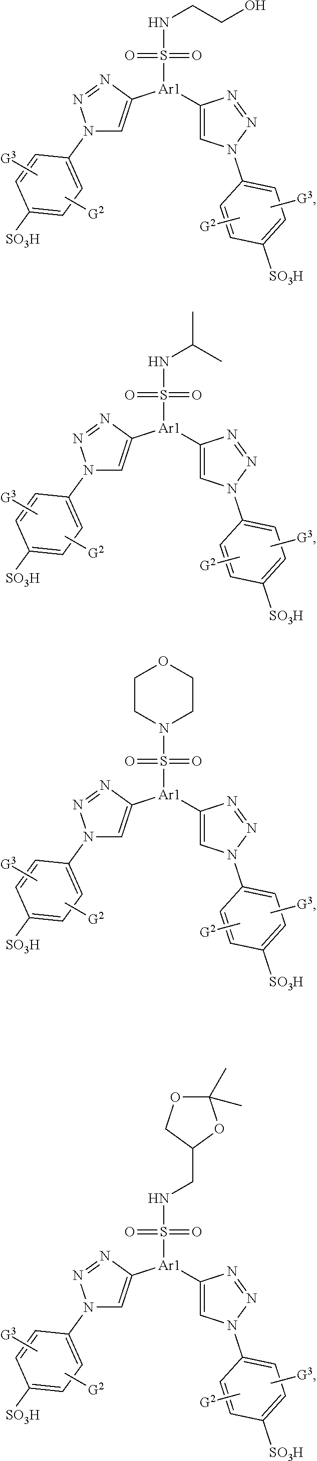

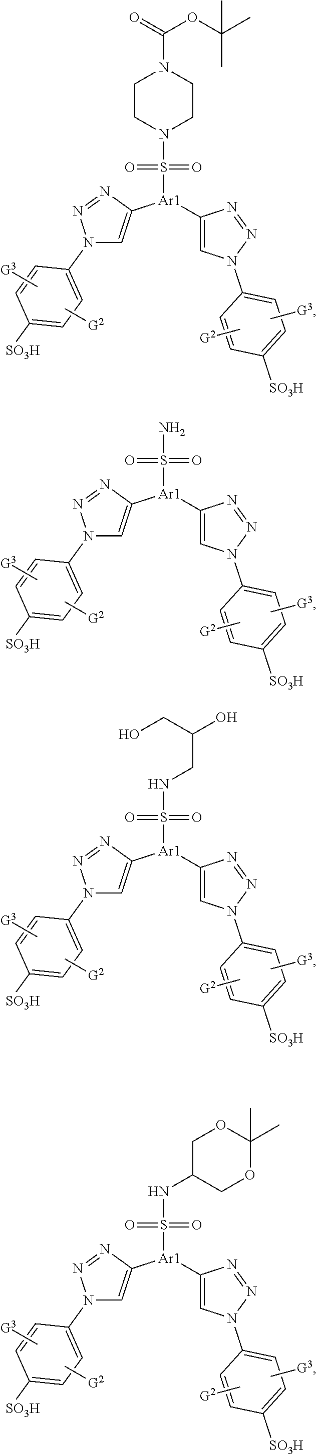

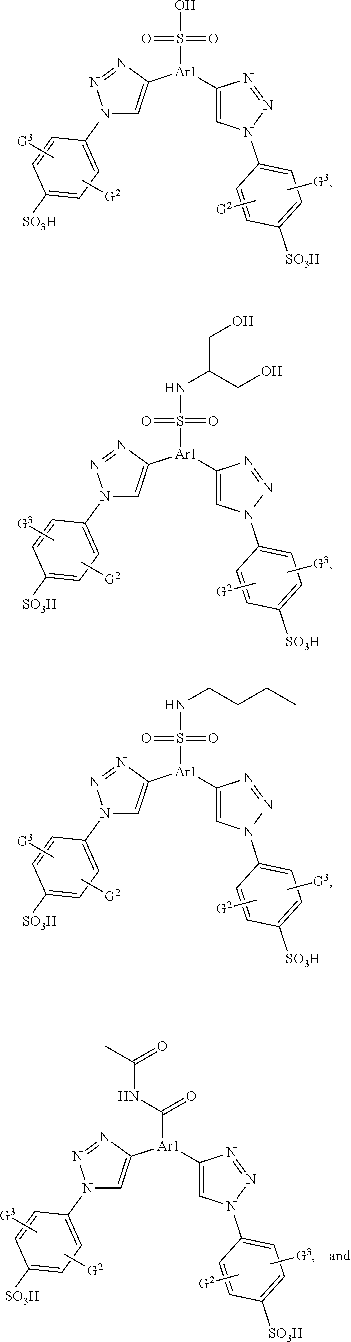

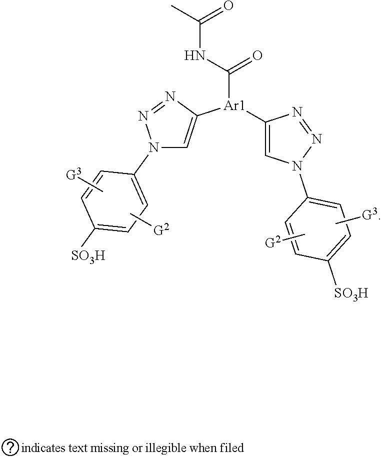

In some embodiments, Ar1 is selected from pyridine, bipyridine, phenyl, and carbazole.

Yet other examples of PEM compounds including substituents on Ar2 include those having the following structures:

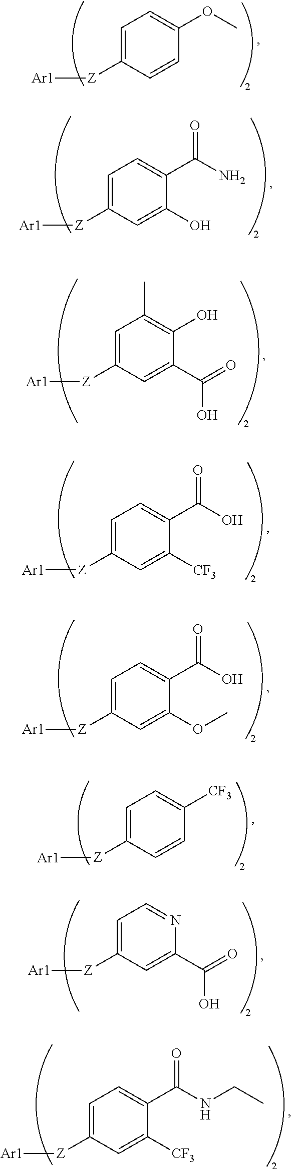

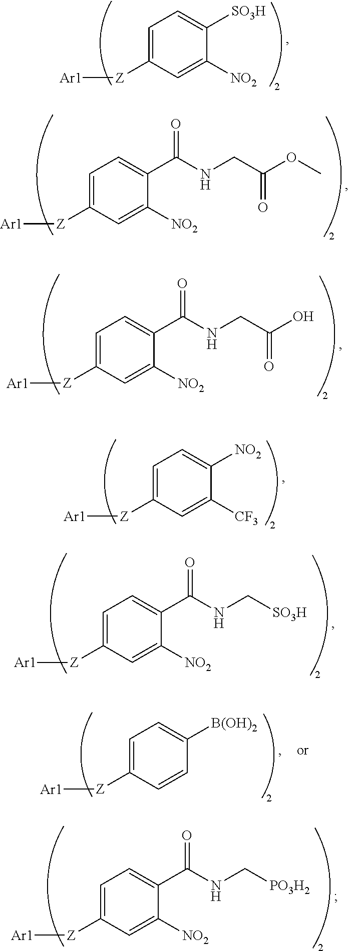

or a salt or hyrdate thereof.

In some embodiments, Z is selected from imidazole, pyrazole, pyrimidine, pyridazine, pyrazine, pyrrole, thiophene, tetrazole, triazole (e.g., 1,2,3-triazole; 1,2,4-triazole, thiazole, and isoxazole. In some embodiments, Z is not a triazole. In some embodiments, in some embodiments, Z is not 1,2,3-triazole or 1,2,4-triazole. In some embodiments, Ar1 is selected from pyridine, bipyridine, phenyl, and carbazole. In some embodiments, the group Ar1 may be substituted (such as with any of the substituents noted herein), or unsubstituted. For instance, Ar1 may be substituted with —C(O)—CH₃, —C(O)—NH₂, —C(O)O—CH₂CH₃, —CF₃, —C(O)O-t-butyl, —C(O)—N(CH₃)₂, —C(O)—N(H) CH₃, —C(O)—N(H)CH₂CH₃, —C(O)—N(H)(cycloalkyl), —C(O)—N(H)(CH₂)₄, —C(O)—N(CH₂CH₃)₂, —C(O)—N(H)(t-butyl), —C(O)—N(H)(C6-cycloalkyl), —C(O)—N(H)(CH₂)₃, —C(O)—N(H)(C5-cycloalkyl), —C(O)—N(H)(C4-cycloalkyl), —C(O)—N(H)(phenyl), —C(O)—N(H)(CH₂CH₂)N(H)C(O)—CH₃, —C(O)—N(H)C(NH)(NH₂), —C(O)—N(CH₂CH₂CH₃)₂, —C(O)—N—(C(H)(CH₃)(CH₂CH₃))₂, —C(O)—N(CH₂CH₂CH₂CH₃)₂, —C(O)—N(H)(benzyl), —C(O)—N(H)—CH₂CH₂OH, —C(O)—S—CH₂CH₃, —C(O)N(CH₂)₂, —N(H)S(O)(O)-phenyl, —C(O)N(H)CH₂CH₂CF₃, —C(O)N(H)CH₃, —C(O)OCH₂CH₃, —C(O)—N(H)—CH₂CH₂O—CH₂CH₂O—CH₃, —C(O)—N(H)—(CH₂CH₂O)₈—CH₃, —C(O)N(H)—CH₂—CH₂—CH₂—N(H)—CH₂—CH₂—CH₂—N(H)—CH₂—CH₂—CH₂—NH₂, —C(O)N(H)—CH₂—CH₂—NH₂, —C(O)N(H)C(O)CH₃, —C(O)N(H)—CH₂—CH₃, —S(O)(O)N(H)—CH₂—CH₃, —S(O)(O)N—(CH₂—CH₃)₂, —S(O)(O)N(H)—(C3-cycloalkyl), —S(O)(O)N(H)—(CH₂CH₂)OH, —S(O)(O)OH, —C(O)N(H)C(H)(CH₂OH)₂, —C(O)N(CH₂CH₂OH)₂, —C(O)N(H)CH₂CH₂═CH₂, —C(O)CH₂N(CH₃)(CH₂)(COOH), —S(O)(O)(CH₂CH₃), —S(O)(O)N(H)CH₂—CH(OH)(CH₂OH), —S(O)(O)—NH₂, -or S(O)(O)N(H)CH₂—C(O)(O-t-butyl).

In some embodiments, the PEM compounds of Formula (I) include solvate, hydrate, chelate, and salt forms thereof. In some instances, the PEM compounds may be amorphous, while in other instances the PEM compounds may be crystalline. Furthermore, some of the crystalline forms of the compounds may exist as polymorphs, which are contemplated herein. In addition, some of the compounds may also form solvates with water or other organic solvents. Such solvates are similarly included within the scope of the compounds described herein.

In some embodiments, the PEM compounds of Formula (I) may be in the form of a chelate, such as a copper chelate. A copper chelate may be formed by combining a PEM compound of the present disclosure with copper sulfate. In some embodiments, the PEM compounds of Formula (I) may be in the form of a salt, either an acid addition salt or a base addition salt, depending on the substituents on the Ar1 and Ar2 groups.

In some embodiments, the PEM structures include all stable stereoisomeric forms thereof. Thus, in some embodiments the PEM compounds described herein may have one or more chiral (or asymmetric) centers and may thus give rise to enantiomers, diastereomers, and other stereoisomeric forms that may be defined, in terms of absolute stereochemistry, as (R)- or (S)-. When the compounds described herein contain olefinic double bonds or other centers of geometric asymmetry, and unless specified otherwise, it is intended that the compounds include both E and Z geometric isomers (e.g., cis or trans). Likewise, unless otherwise indicated, all possible isomers, as well as their racemic and optically pure forms, and all tautomeric forms are also intended to be included. It is therefore contemplated that various stereoisomers and mixtures thereof include “enantiomers,” which refers to two stereoisomers whose molecules are non-superimposable mirror images of one another. Thus, the compounds may occur in any isomeric form, including racemates, racemic mixtures, and as individual enantiomers or diastereomers.

In some embodiments, the present disclosure provides for polymers of the PEM compounds described herein. In some embodiments, the PEM compounds disclosed herein serve as monomers which may be polymerized into polymers (e.g., via cationic polymerization, such as by incubating a PEM with KCl). As used herein, the term “polymer” is defined as being inclusive of homopolymers, copolymers, and oligomers. The term “homopolymer” is defined as a polymer derived from a single species of monomer, e.g., a single PEM monomer species. The term “copolymer” is defined as a polymer derived from more than one species of monomer, including copolymers that are obtained by copolymerization of two monomer species (such as two different PEM monomer species). The term “oligomer” is defined as a low molecular weight polymer in which the number of repeating units does not exceed twenty. In some embodiments, the polymers of the present disclosure include two repeating units, such as three repeating units, such as four repeating units, such as five repeating units, such as six repeating units, etc. In some embodiments, the polymers, copolymers, and/or oligomers are formed through covalent, ionic, or weak interactions.

In some embodiments, the present disclosure provides for compositions including two or more molecules of any one of the compounds disclosed herein (such as two or more compounds having any one of Formulas (I), (IA), (IB), (IC), and/or (ID)). In some embodiments, the compositions include 10 or more molecules of any of the compounds disclosed herein, such as 20 or more molecules of any of the compounds disclosed herein, such as 50 or more molecules of any of the compounds disclosed herein, such as 100 or more molecules of any of the compounds disclosed herein, such as 200 or more molecules of any of the compounds disclosed herein, etc.

In some embodiments, the PEM compounds of the present disclosure generally are water soluble. One measure of water solubility is the logP value of a compound. LogP values may be calculated using commercial software, based on the chemical structure of the compound. For instance, the CHEMDRAW chemical drawing software (Cambridgesoft Limited, a subsidiary of PerkinElmer Holdings) can calculate a logP value for a drawn chemical structure. In one embodiment, a PEM compound of the present disclosure has a logP of at least 4.9.

In some embodiments, the compounds of the present disclosure, for example, PEM compounds of any one of Formulas (I,), (IA), (IB), (IC), and (ID) as described herein, may typically be synthesized by the reaction of diethynyl compounds of the formula Ar1(C≡CH)2 with azide compounds of the formula Ar2-N3, such as in the presence of Cu(I) catalyst. See also Crowley J. D., McMorran D. A. (2012) “Click-Triazole” Coordination Chemistry: Exploiting 1,4-Disubstituted-1,2,3-Triazoles as Ligands. In: Kosmrlj J. (eds.) Click Triazoles. Topics in Heterocyclic Chemistry, vol. 28. Springer, Berlin, Heidelberg doi.org/10.1007/7081_2011_67.

In some embodiments, non-limiting examples of suitable compounds of the formula Ar1(C≡CH)₂ include:

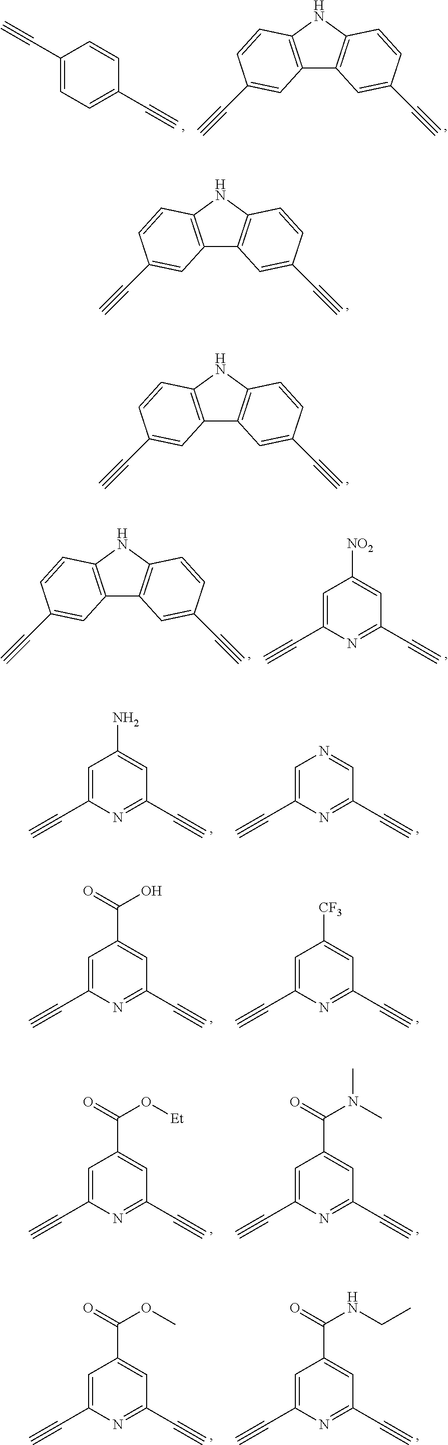

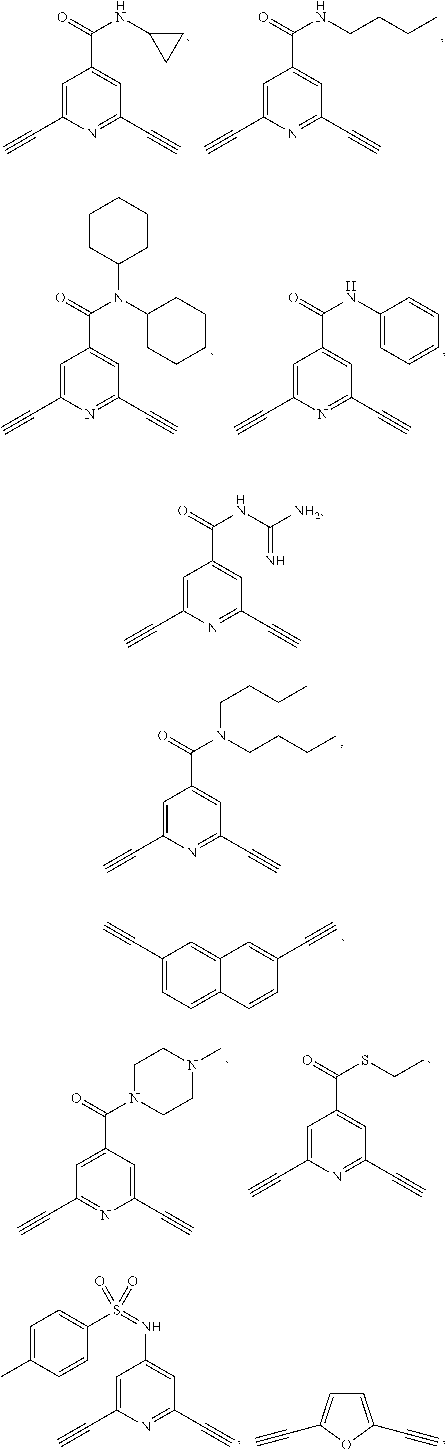

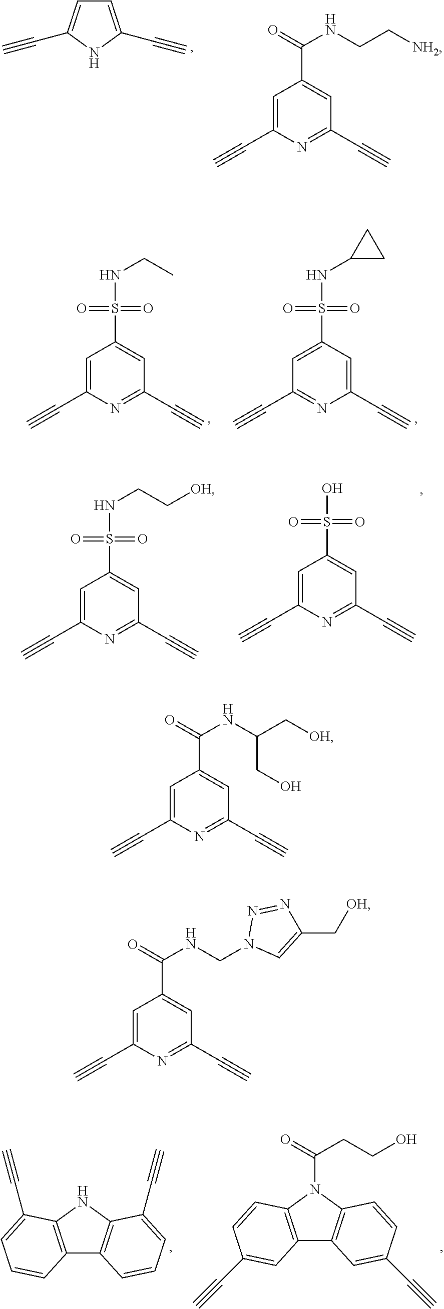

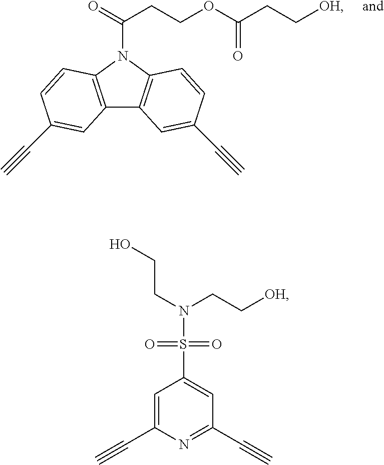 [SMILES: C#Cc1ccc2c(c1)c1cc(C#C)ccc1n2C(=O)CCOC(=O)CCO ; "and" ; C#Cc1cc(S(=O)(=O)N(CCO)CCO)cc(C#C)n1]

In other embodiments, compounds of the formula Ar1(C≡CH)₂ include:

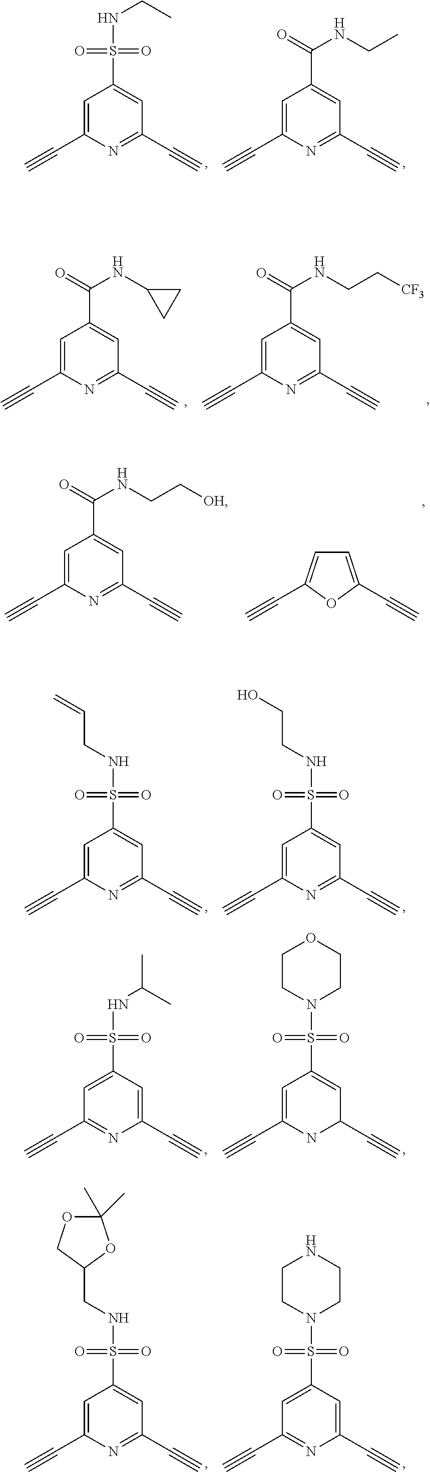

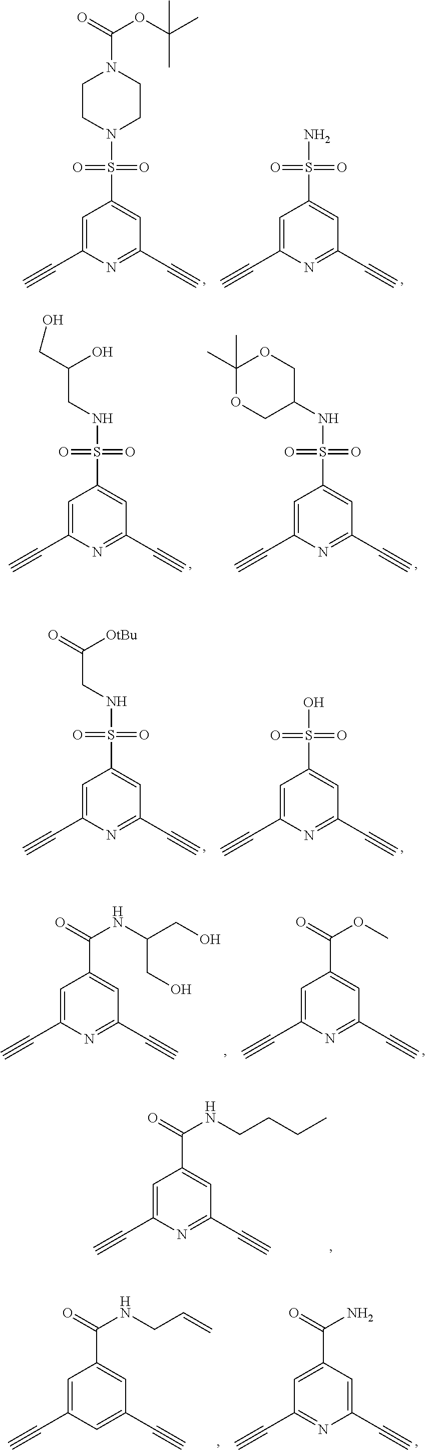

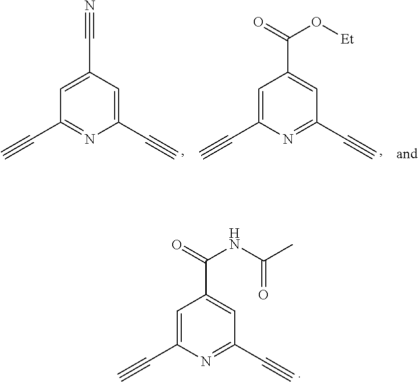

Non-limiting examples of suitable compounds of the formula Ar2-N₃ include:

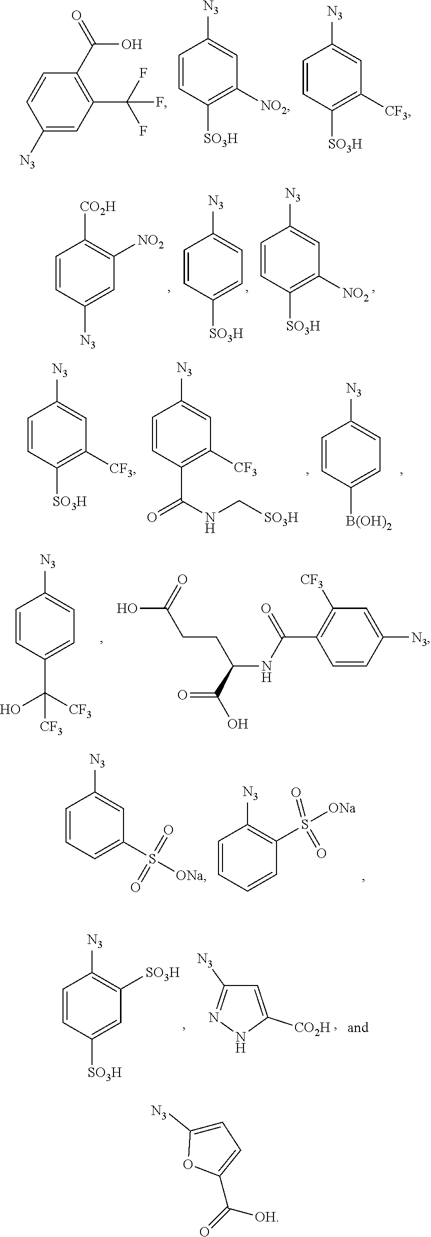

Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002.

Compounds of the formula Ar1(C≡CH) are commercially available, e.g., from TCI America (Portland, Oregon, USA), which sells, e.g., 1,3-diethynylbenzene, 1,4-diethynylbenzene, 2,6-diethynylpyridine and 3,6-diethynylcarbazole.

In general, ethynyl aromatic compounds may be prepared via a Seyferth-Gilbert homologation from an aryl aldehyde using dimethyl (diazomethyl) phosphonate available from MilliporeSigma Corp. (St. Louis, MO, USA). Alternatively, dimethyl (diazomethyl)phosphonate can be generated in situ from dimethyl-1-diazo-2-oxopropylphosphonate (Ohira-Bestmann reagent). See, e.g., Seyforth et al., J. Org. Chem. 36(10): 1379-1386 (1971). doi:10.1021/jo00809a014 and Bestman et al., Synlett. 1996 (06): 521-522 (1996). doi:10.1055/s-1996-5474.

Another route to ethynyl aromatic compounds entails a Sonogashira coupling of halo aromatic compounds with (t-butyldimethylsilyl)acetylene in the presence of a palladium catalyst. The ethynyl aromatic forms upon subsequent deprotection the silyl group. See, e.g., Sonogashira, Organomet. Chem., 653: 46-49 (2002). doi:10.1016/s0022-328x(02)01158-0.

The following reactions (I), (II) and (III) illustrate exemplary preparations of diethynyl aromatic compounds. In reaction (I), 2,6-dibromopyridin-4-amine is converted to the corresponding 2,6-diethynylpyridin-4-amine compound. In reaction (II), 2,6-diiodo-4-nitroanilie is converted to the corresponding 2,6-diethynyl-4-nitroaniline. In reaction (III), 2-hydroxy-3,5-diiodobenzoic acid is converted to the corresponding 3,5-diethynyl-2-hydroxybenzoic acid. In each case, the conversion proceeds through the intermediate di-trimethylsilyl (TMS) compound as shown.

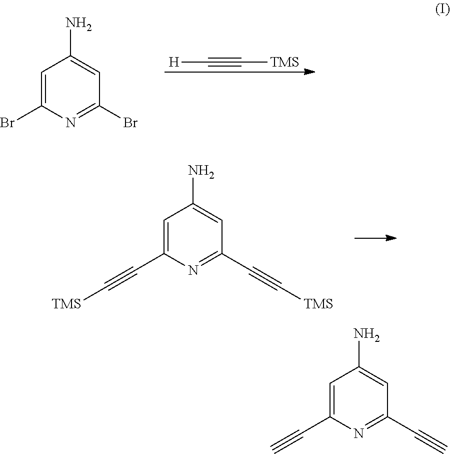 [SMILES: Nc1cc(Br)nc(Br)c1 ; C#C[Si](C)(C)C ; C[Si](C)(C)C#Cc1cc(N)cc(C#C[Si](C)(C)C)n1 ; C#Cc1cc(N)cc(C#C)n1]

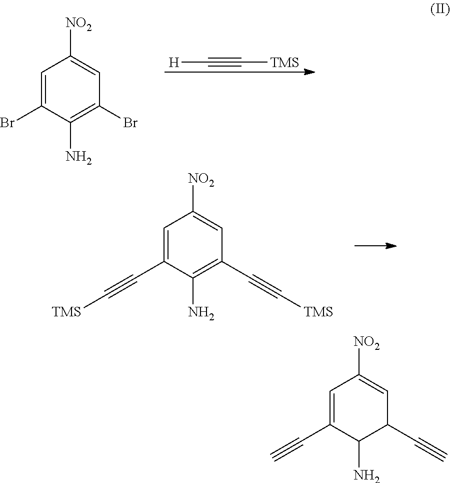 [SMILES: Nc1c(Br)cc([N+](=O)[O-])cc1Br ; C#C[Si](C)(C)C ; C[Si](C)(C)C#Cc1cc([N+](=O)[O-])cc(C#C[Si](C)(C)C)c1N ; C#CC1=CC([N+](=O)[O-])=CC(C#C)C1N]

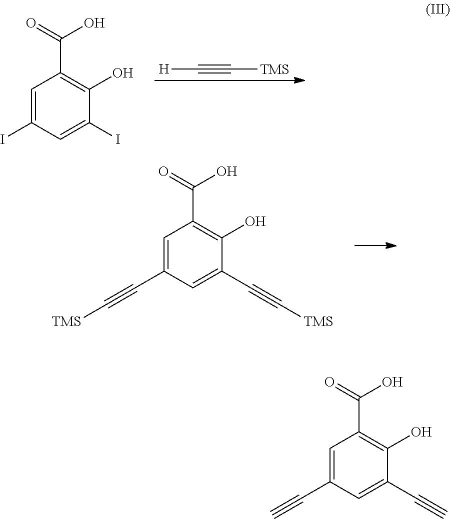

Each of these reaction products, namely 2,6-diethynylpyridin-4-amine, and 2,6-diethynyl-4-nitroaniline, and 3,5-diethynyl-2-hydroxybenzoic acid, may function as the precursor to Ar1 in preparing PEMs of the present disclosure. Thus, each of them represents an Ar1(C≡CH)₂ compound which may be reacted with an azide compound of the formula Ar2-N₃ in the presence of Cu(I) catalyst to provide a PEM. The reactions (I), (II) and (III) illustrate the preparation of a precursor to a substituted Ar1 moiety of the present disclosure.

Compounds of the formula Ar2-N₃ are likewise commercially available, e.g., from TCI America (Portland, Oregon, USA), Synthonix (Wake Forest, North Carolina, USA), SigmaAldrich (St. Louis, Missouri, USA), Toronto Research Chemicals (Toronto, Canada), and AnaSpec (Fremont, California, USA). In general, azides of the formula Ar2-N₃ may be prepared by nucleophilic displacement with sodium azide of electrophilic compounds such as an alkyl, benzylic or allylic iodide or bromide.

In general, the compounds used in the reactions described herein may be made according to organic synthesis techniques known to those skilled in this art, starting from commercially available chemicals and/or from compounds described in the chemical literature. “Commercially available chemicals” may be obtained from standard commercial sources including Across Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hanover, Germany), Spectrum Quality Product, Inc. (New Brunswick, N.J.), TCI America (Portland Oreg.), Trans World Chemicals, Inc. (Rockville Md.), and Wako Chemicals USA, Inc. (Richmond Va.).

In one embodiment, a PEM compound of the present disclosure, e.g., a PEM compound of formula (I) is present in a composition. For example, the PEM compounds of the present disclosure may be present in a composition also comprising an aqueous buffer. In one embodiment, the PEM compounds of the present disclosure are present in a composition comprising a biomolecule such as a polypeptide and/or a polynucleotide. The polypeptide may be an enzyme such as a DNA polymerase. The following definitions may be helpful to an understanding of these compositions and certain uses thereof.

As used herein, “nucleic acids,” also referred to as polynucleotides, are covalently linked series of nucleotides in which the 3′ position of the pentose of one nucleotide is joined by a phosphodiester group to the 5′ position of the next. A nucleic acid molecule can be deoxyribonucleic acid (DNA), ribonucleic acid (RNA), or a combination of both. DNA (deoxyribonucleic acid) and RNA (ribonucleic acid) are biologically occurring polynucleotides in which the nucleotide residues are linked in a specific sequence by phosphodiester linkages. As used herein, the terms “nucleic acid”, “polynucleotide” or “oligonucleotide” encompass any polymer compound having a linear backbone of nucleotides. Oligonucleotides, also termed oligomers, are generally shorter chained polynucleotides. Nucleic acids are generally referred to as “target nucleic acids” or “target sequence” if targeted for sequencing.

As used herein, the term “template dependent manner” is intended to refer to a process that involves the template dependent extension of a primer molecule (e.g., DNA synthesis by DNA polymerase). The term “template dependent manner” refers to polynucleotide synthesis of RNA or DNA wherein the sequence of the newly synthesized strand of polynucleotide is dictated by the well-known rules of complementary base pairing (see, for example, Watson, J. D. et al., In: Molecular Biology of the Gene, 4th Ed., W. A. Benjamin, Inc., Menlo Park, Calif. (1987)).

As used herein, “nucleic acid polymerase” is an enzyme generally for joining 3′-OH 5′-triphosphate nucleotides, oligomers, and their analogs. Polymerases include, but are not limited to, DNA-dependent DNA polymerases, DNA-dependent RNA polymerases, RNA-dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase 1, Klenow fragment, Thermophilus aquaticus DNA polymerase, Tth DNA polymerase, VentR® DNA polymerase (New England Biolabs), Deep VentR® DNA polymerase (New England Biolabs), Bst DNA Polymerase Large Fragment, Stoeffel Fragment, 9° N DNA Polymerase, 9° N DNA polymerase, Pfu DNA Polymerase, Tfl DNA Polymerase, Tth DNA Polymerase, RepliPHI Phi29 Polymerase, Tli DNA polymerase, eukaryotic DNA polymerase beta, telomerase, Therminator™ polymerase (New England Biolabs), KOD HiFi™ DNA polymerase (Novagen), KOD1 DNA polymerase, Q-beta replicase, terminal transferase, AMV reverse transcriptase, M-MLV reverse transcriptase, Phi6 reverse transcriptase, HIV-1 reverse transcriptase. A polymerase according to the present disclosure can be a variant, mutant, or chimeric polymerase.

As used herein, a “DP04-type DNA polymerase” is a DNA polymerase naturally expressed by the archaea, Sulfolobus solfataricus, or a related Y-family DNA polymerase, which generally function in the replication of damaged DNA by a process known as translesion synthesis (TLS). Y-family DNA polymerases are homologous to the DP04 polymerase; examples include the prokaryotic enzymes, PolII, PolIV, PolV, the archaeal enzyme, Dbh, and the eukaryotic enzymes, Rev3p, Revlp, Pol η, REV3, REV1, Pol 1, and Pol K DNA polymerases, as well as chimeras thereof. A modified recombinant DP04-type DNA polymerase includes one or more mutations relative to naturally-occurring wild-type DPO4-type DNA polymerases, for example, one or more mutations that increase the ability to utilize bulky nucleotide analogs as substrates or another polymerase property, and may include additional alterations or modifications over the wild-type DPO4-type DNA polymerase, such as one or more deletions, insertions, and/or fusions of additional peptide or protein sequences (e.g., for immobilizing the polymerase on a surface or otherwise tagging the polymerase enzyme). Examples of variant polymerase according to the present disclosure are the variants of Sulfolobus sulfataricus DP04 described in published PCT patent application WO2017/087281 A1 and PCT patent applications nos. PCTUS2018/030972 and PCTUS2018/64794 which are hereby incorporated by reference in their entirety.

As used herein, “nucleic acid polymerase reaction” refers to an in vitro method for making a new strand of nucleic acid or elongating an existing nucleic acid (e.g., DNA or RNA) in a template dependent manner. Nucleic acid polymerase reactions, according to the present disclosure, includes primer extension reactions, which result in the incorporation of nucleotides or nucleotide analogs to a 3′-end of the primer such that the incorporated nucleotide or nucleotide analog is complementary to the corresponding nucleotide of the target polynucleotide. The primer extension product of the nucleic acid polymerase reaction can further be used for single molecule sequencing or as templates to synthesize additional nucleic acid molecules.

Primer extension reaction reagents typically include (i) a polymerase enzyme; (ii) a buffer; and (iii) one or more extendible nucleotides or nucleotide analogs. Primer extension reactions can be used to measure the length of a resulting nucleic acid product under particular experimental conditions and to determine the effect of various polymerase reaction additives (e.g., PEMs) on polymerase activity by comparing the lengths of the extended primer products by, e.g., gel electrophoresis.

As used herein, “enhancing a nucleic acid polymerase reaction” refers to the ability of an additive, e.g., a PEM to enable a nucleic acid polymerase to synthesize a primer extension product at least one subunit longer in length than it would in the absence of the PEM.

The rate of a nucleic acid polymerase reaction as used herein refers to the average speed at which a nucleic acid polymerase extends a polymer chain. As used herein, the terms “speed” and “elongation rate” are used inter-changeably. The nucleotide incorporation assay of Hogrefe et al. (Methods in Enzymol. Vol. 334, pp. 91-116 (2001)) can be used to measure the rate of polymerization. Briefly, polymerase activity can be measured as the rate of incorporation of ³²P-dCTP into activated salmon sperm DNA (purchased from Pharmacia; for activation protocol see C. C. Richardson, Procedures in Nucl. Acid Res. (Cantoni and Davies, eds.), p. 263-276 (1966) at p. 264). The reaction buffer can be, for example, 50 mM Tris-HCl (pH 8.0), 5 mM MgCl₂, 1 mM dithiothreitol (DTT), 50 μg/ml bovine serum albumin (BSA), and 4% (v/v) glycerol. Nucleotide substrates and DNA are used in large excess, typically at least 10 times the Km for the polymerase being assayed, e.g., 200 μM each of dATP, dTTP, and dGTP, 195 μM of dCTP plus 5 μIM of labeled dCTP, and 250 μg/ml of activated DNA. The reactions are quenched on ice, and aliquots of the reaction mixture are spotted onto ion exchange filters (e.g., Whatman DE81). Unincorporated nucleotide is washed through, followed by scintillation counting to measure incorporated radioactivity.

As used herein, “increasing the rate” refers to an increase of 5-10%, 5-20%, 10-50%, or 50-100% or more, as compared to a polymerization reaction that lacks a PEM that increases rate as defined herein.

As used herein, “processivity” refers to the extent of polymerization by a nucleic acid polymerase during a single contact between the polymerase and its template, i.e., its property to continue to act on a substrate instead of dissociating therefrom. The extent of polymerization refers to the number of nucleotides or nucleotide analogs added by the polymerase during a single contact between the polymerase and its template. Processivity can depend on the nature of the polymerase, the sequence of a template, the structure of the nucleotide or nucleotide analog substrates, and the reaction conditions, for example, salt concentration, temperature, or the presence of specific additives.

As used herein, “increasing the processivity” refers to an increase of 5-10%, 5-20%, 10-50%, or 50-100% or more, as compared to a polymerization reaction that lacks a PEM that increases processivity as defined herein. Methods for measuring processivity of a nucleic acid polymerase are generally known in the art, e.g., as described in Sambrook et al. 1989, In Molecular Cloning, 2nd Edition, CSH Press, 7.79-7.83 and 13.8, as described in U.S. published patent application no. 2002/0119467, published PCT application no. WO01/92501 and in U.S. Pat. No. 5,972,603, the entireties of which are incorporated herein by reference.

The term “fidelity” as used herein refers to the accuracy of nucleic acid polymerization by template-dependent nucleic acid polymerase. The fidelity of a DNA polymerase is measured by the error rate (the frequency of incorporating an inaccurate nucleotide, i.e., a nucleotide that is not incorporated at a template-dependent manner). The fidelity or error rate of a DNA polymerase may be measured using assays known to the art (see for example, Lundburg et al., 1991 Gene, 108:1-6). As used herein, “increasing the fidelity” refers to an increase of 5-10%, 10-50%, or 50-100% or more, as compared to a polymerization reaction that lacks an additive that increases fidelity as defined herein.

The term “plurality” as used herein refers to “at least two.”

“XNTP” is an expandable, 5′ triphosphate modified nucleotide substrate compatible with template dependent enzymatic polymerization. An XNTP has two distinct functional components; namely, a nucleobase 5′-triphosphoramidate and a tether that is attached within each nucleoside triphosphoramidate at positions that allow for controlled expansion by intra-nucleotide cleavage of the phosphoramidate bond. XNTPs are exemplary “non-natural, highly substituted nucleotide analog substrates”, as used herein. Exemplary XNTPs and methods of making the same are described, e.g., in Applicants' published PCT application no. WO2016/081871, herein incorporated by reference in its entirety.

“Xpandomer intermediate” is an intermediate product (also referred to herein as a “daughter strand”) assembled from XNTPs and is formed by polymerase-mediated template-directed assembly of XNTPs using a target nucleic acid template. The newly synthesized Xpandomer intermediate is a constrained Xpandomer. Under a process step in which the phosphoramidate bonds provided by the XNTPs are cleaved, the constrained Xpandomer is no longer constrained and is the Xpandomer product which is extended as the tethers are stretched out.

“Xpandomer” or “Xpandomer product” is a synthetic molecular construct produced by expansion of a constrained Xpandomer, which is itself synthesized by template-directed assembly of XNTP substrates. The Xpandomer is elongated relative to the target template it was produced from. It is composed of a concatenation of subunits, each subunit a motif, each motif a member of a library, comprising sequence information, a tether and optionally, a portion, or all of the substrate, all of which are derived from the formative substrate construct. The Xpandomer is designed to expand to be longer than the target template thereby lowering the linear density of the sequence information of the target template along its length. In addition, the Xpandomer optionally provides a platform for increasing the size and abundance of reporters which in turn improves signal to noise for detection. Lower linear information density and stronger signals increase the resolution and reduce sensitivity requirements to detect and decode the sequence of the template strand.

“Tether” or “tether member” refers to a polymer or molecular construct having a generally linear dimension and with an end moiety at each of two opposing ends. A tether is attached to a nucleoside triphosphoramidate with a linkage at end moiety to form an XNTP. The linkages serve to constrain the tether in a “constrained configuration”. Tethers have a “constrained configuration” and an “expanded configuration”. The constrained configuration is found in XNTPs and in the daughter strand, or Xpandomer intermediate. The constrained configuration of the tether is the precursor to the expanded configuration, as found in Xpandomer products. The transition from the constrained configuration to the expanded configuration results cleaving of selectively cleavable phosphoramidate bonds. Tethers comprise one or more reporters or reporter constructs along its length that can encode sequence information of substrates. The tether provides a means to expand the length of the Xpandomer and thereby lower the sequence information linear density.

“Tether element” or “tether segment” is a polymer having a generally linear dimension with two terminal ends, where the ends form end-linkages for concatenating the tether elements. Tether elements are segments of tether. Such polymers can include, but are not limited to: polyethylene glycols, polyglycols, polypyridines, polyisocyanides, polyisocyanates, poly(triarylmethyl)methacrylates, polyaldehydes, polypyrrolinones, polyureas, polyglycol phosphodiesters, polyacrylates, polymethacrylates, polyacrylamides, polyvinyl esters, polystyrenes, polyamides, polyurethanes, polycarbonates, polybutyrates, polybutadienes, polybutyrolactones, polypyrrolidinones, polyvinylphosphonates, polyacetamides, polysaccharides, polyhyaluranates, polyamides, polyimides, polyesters, polyethylenes, polypropylenes, polystyrenes, polycarbonates, polyterephthalates, polysilanes, polyurethanes, polyethers, polyamino acids, polyglycines, polyprolines, N-substituted polylysine, polypeptides, side-chain N-substituted peptides, poly-N-substituted glycine, peptoids, side-chain carboxyl-substituted peptides, homopeptides, oligonucleotides, ribonucleic acid oligonucleotides, deoxynucleic acid oligonucleotides, oligonucleotides modified to prevent Watson-Crick base pairing, oligonucleotide analogs, polycytidylic acid, polyadenylic acid, polyuridylic acid, polythymidine, polyphosphate, polynucleotides, polyribonucleotides, polyethylene glycol-phosphodiesters, peptide polynucleotide analogues, threosyl-polynucleotide analogues, glycol-polynucleotide analogues, morpholino-polynucleotide analogues, locked nucleotide oligomer analogues, polypeptide analogues, branched polymers, comb polymers, star polymers, dendritic polymers, random, gradient and block copolymers, anionic polymers, cationic polymers, polymers forming stem-loops, rigid segments and flexible segments.

A “reporter” is composed of one or more reporter elements. Reporters serve to parse the genetic information of the target nucleic acid.

“Reporter construct” comprises one or more reporters that can produce a detectable signal(s), wherein the detectable signal(s) generally contain sequence information. This signal information is termed the “reporter code” and is subsequently decoded into genetic sequence data. A reporter construct may also comprise tether segments or other architectural components including polymers, graft copolymers, block copolymers, affinity ligands, oligomers, haptens, aptamers, dendrimers, linkage groups or affinity binding group (e.g., biotin).

“Reporter Code” is the genetic information from a measured signal of a reporter construct. The reporter code is decoded to provide sequence-specific genetic information data.

Thus, in some embodiments the present disclosure provides a composition comprising a PEM as disclosed herein and a buffer. In other embodiments, the present disclosure provides a composition comprising a PEM as disclosed herein and a plurality of nucleotides and/or nucleotide analogs. In other embodiments, the present disclosure provides a composition comprising a PEM as disclosed herein and a polynucleotide. In other embodiments, the present disclosure provides a composition comprising a PEM as disclosed herein and a protein, where optionally the protein is a polymerase including any of the polymerases described above.

In some embodiments, the composition further comprises one or more component selected from MnCl₂, a buffer, a salt, a sugar, a single-strand binding protein (SSB), imidazole, pyrazole, triazole, betaine, a molecular crowding agent, dimethyl sulfoxide (DMSO), an alkanediol, glycerol, N-methyl-2-pyrrolidone (NMP), acetamide, butylated hydroxyanisole (BHA), a polyphosphate, urea, and a mixture of nucleotides or nucleotide analogs.

In some embodiments, the salt is salt is selected from the group consisting of NaCl, NaBr, NaOAc, NaF, sodium formate, sodium phosphate monobasic, sodium phosphate dibasic, NaSO₄, sodium carbonate, sodium bicarbonate, sodium hexanoate, sodium glutamate, sodium perchlorate, CsCl, LiCl, LiOAc, LiF, lithium carbonate, LiPO₄, KCl, KOAc, KF, KSO₄, potassium phosphate monobasic, potassium phosphate dibasic, potassium carbonate, potassium bicarbonate, potassium glutamate, NH₄Cl, NH₄F, NH₄OAc, NH₄SO₄, NH₄Br, ammonium citrate, ammonium carbonate, ammonium bicarbonate, ammonium sulfite, ammonium glutamate, ammonium phosphate monobasic, tetramethylammonium chloride (TMACl), trimethylamine N-oxide (TMAO), tetraethylammonium chloride (TEACl), guanidinium chloride, guanidinium thiocyanate, guanidium carbonate. In some embodiments, the salt is an inorganic salt. In some embodiments, the salt is NaCl or KCl, such as NaCl.

In some embodiments, the sugar is maltose, trehalose, cellobiose or sucrose.

In some embodiments, the SSB is selected from the group consisting of KOD (SEQ ID NO: 6), Gp32 (SEQ ID NO: 7), TTH (SEQ ID NO: 8), SSB1 (SEQ ID NO: 9), RecA (SEQ ID NO: 10), RPA (SEQ ID NO: 11), and NCp7 (SEQ ID NO: 12).

In some embodiments, the imidazole includes (apart from imidazole as such) imidazole derivatives, such as imidazole chloride, imidazole acetate, 1-methylimidazole, 2-methylimidazole, 1-ethylimidazole, 1-ethyl-3-methylimidazolium chloride, 2-methyl-2-imidazoline, 1-butyl-3-methylimidazolium chloride, 1-methylimidazolium chloride, 1-hexyl-3-methylimidazolium, 3-octyl-1-methylimidazolium, or 1-decyl-3-methylimidazolium.

In some embodiments, the alkanediol is ethylenglycol, a propanediol or a butanediol, such as a propanediol.

In some embodiments, the propanediol is 1,2-propanediol or 1,3-propanediol, such as 1,2 propanediol. In some embodiments, the butanediol is 1,2-butanediol, 1,3-butanediol, 1,4-butanediol, 2,3-butanediol, 2,4-butanediol, or 3,4-butanediol, such as 1,2-butanediol.

In some embodiments, the molecular crowding agent is PEG, such as PEG4k to PEG25k or PEG4k to PEG 10k, such as PEG5k, PEG8k, or PEG10k.

In some embodiments, the polyphosphate is tripolyphosphate, tetrapolyphosphate, pentapolyphosphate, hexapolyphosphate, trimetaphosphate (TMP), hexametaphosphate (HMP), or polyphosphate 60.

In one embodiment, the present disclosure provides a composition comprising a PEM compound of the present disclosure, e.g., a PEM compound of any one of Formulas (I), (IA), (IB), (IC), and (ID) and a molecular crowding agent. In general terms, molecular crowding agents include a range of large, neutral polymers. Examples of useful molecular crowding reagents include, but are not limited to, polyethylene glycol (PEG), ficoll, dextran, or polyvinyl alcohol. Exemplary molecular crowding reagents and formulations are set forth in U.S. Pat. No. 7,399,590, which is incorporated herein by reference. In one embodiment, the molecular crowding agent is a polyalkylene glycol, optionally having a number average molecular weight of 4,000-10,000. In one embodiment, the molecular crowing agent is a derivative of a polyalkylene glycol, e.g., one or both of the terminal hydroxyl groups of a polyalkylene glycol is in the form of an ester or ether group. In some embodiments, the polyalkylene glycol is polyethylene glycol. In one embodiment, the molecular crowding agent is an inert, water-soluble polymer.

In one embodiment, the present disclosure provides a composition comprising a PEM compound of the present disclosure and an aqueous buffer. In one embodiment the PEM compound has any one of Formulas (I), (IA), (IB), (IC), and (ID). In one option, the composition has a pH of about 6 to 8.5, and the buffer helps to stabilize the pH of the composition. An exemplary buffer is Tris HCl. Other suitable buffers include those known in the art, e.g., phosphate buffers, citric acid buffers, sodium acetate buffers, sodium carbonate buffers, and the like. In some embodiments, the buffer is selected from the group consisting of TrisCl, TrisOAc, NH₄OAc, MES, and HEPES.

In one embodiment, the present disclosure provides a composition comprising a PEM compound of the present disclosure, e.g., a PEM compound of any one of Formulas (I), (IA), (IB), (IC), and (ID), and a polynucleotide. In one option, the polynucleotide is single stranded, e.g., single stranded DNA or a single stranded RNA. When the polynucleotide is intended to function as a primer, the polynucleotide is a single stranded DNA molecule. When intended to function as a primer, the polynucleotide may have a length of about 10-60 mer oligonucleotide, e.g., 20-30 oligonucleotides. The polynucleotide may alternatively function as a template, in which case it may be a single stranded DNA or a single stranded RNA and may have a length of from 30 bases to kilobase and above values, e.g., 10k bases and above.

In one embodiment, the present disclosure provides a composition comprising a PEM compound of the present disclosure, e.g., a PEM compound of any one of Formulas (I), (IA), (IB), (IC), and (ID), and a protein. For example, the protein may be an enzyme, a nucleic acid polymerase, a DNA polymerase. One example of a suitable DNA polymerase is a variant of DP04 polymerase, as discussed herein.

In one embodiment, the present disclosure provides a composition comprising at least one PEM compound of the present disclosure, e.g., a PEM compound having any one of Formulas (I), (IA), (IB), (IC), and (ID), and a mixture of nucleotides or nucleotide analogs wherein the at least one compound increases the number and accuracy of nucleotide analogs incorporated into a daughter strand during a template-dependent polymerization reaction relative to an identical polymerization reaction absent the at least one compound. Optionally, the mixture of nucleotide analogs includes nucleoside triphosphoramidates, wherein each of the nucleoside triphosphoramidates has a nucleobase selected from adenine, guanine, thymine, and cytosine and a polymeric tether moiety, wherein a first end of the polymeric tether moiety is attached to the nucleobase and a second end of the polymeric ether moiety is attached to the alpha phosphate of the nucleoside triphosphoramidate to provide for expansion of the nucleotide analogs by cleavage of the phosphoramidate bond. Optionally, the composition further includes a buffer which includes one or more components selected from MnCl₂, a buffer, a salt, a sugar, a single-strand binding protein (SSB), imidazole, pyrazole, triazole, betaine, a molecular crowding agent, dimethyl sulfoxide (DMSO), an alkanediol, glycerol, N-methyl-2-pyrrolidone (NMP), acetamide, butylated hydroxyanisole (BHA), a polyphosphate, and/or urea. Optionally, the composition also includes a single-strand binding protein. Optionally, the composition includes urea. Optionally, the mixture of nucleotide analogs includes nucleotide analogs that comprise a detectable label, where the detectable label is optionally one of luminescent, chemiluminescent, fluorescent, fluorogenic, chromophoric or chromogenic. In one embodiment, the composition includes two or more of these options, e.g., all, of these options.

In one aspect of the present disclosure, PEMs and compositions thereof as disclosed herein may be used to enhance a nucleic acid polymerization reaction or improve the properties of the resulting nucleic acid, e.g., the length or accuracy of the reaction product. Polymerization reactions include, e.g., primer extension reactions, PCR, mutagenesis, isothermal amplification, DNA sequencing, and probe labeling. Such methods are well known in the art. Enhancement may be provided by stimulating nucleotide incorporation through mechanisms such as increasing processivity of the polymerase (i.e., reducing dissociation of the polymerase from the template), increasing the rate of substrate binding or enzymatic catalysis, and increasing the accuracy or fidelity of nucleotide incorporation. In addition, enhancement may be provided by reducing impediments in the nucleic acid template, such as secondary structure and duplex DNA. Overcoming or improving such impediments through the addition of PEMs can allow polymerization reactions to occur more accurately or efficiently or allow the use of lower denaturation/extension temperatures or isothermal temperatures.

In some embodiments, a PEM may be used in combination with another additive classes to enhance a polymerase reaction. One exemplary class of additives is minor groove binding proteins (MGBs). In one embodiment, the MGB is selected from the group consisting of distamycin A and synthetic analogs thereof, netropsin, (+)—CC-1065, duocarmycins, pyrrolobenzodiazepines, trabectin and analogs thereof, Hoechst dyes and derivatives thereof, lexitropsin, thiazotropsin A, diamidines, and polyamides. In certain embodiments, the at least one minor groove binding moiety is a Hoechst dye. More information about the use of MGBs to enhance a polymerase reaction may be found in applicants' co-filed application titled ENHANCEMENT OF NUCLEIC ACID POLYMERIZATION BY MGBS.

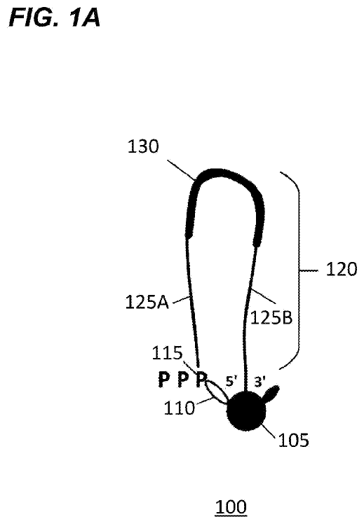

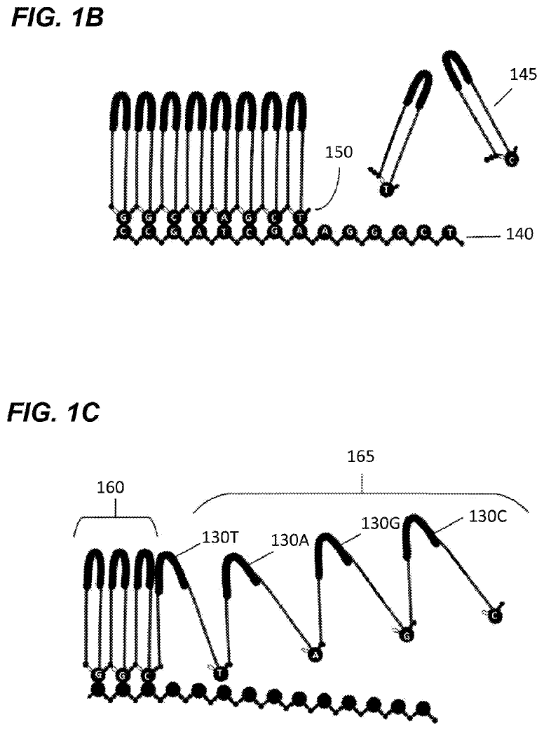

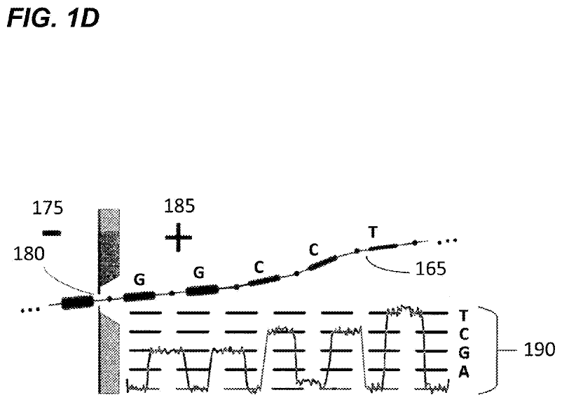

One exemplary polymerase reaction that can be enhanced with PEMs is the polymerization of the non-natural nucleotide analogs known as “XNTPs”, which forms the basis of the “Sequencing by Expansion” (SBX) protocol, developed by Stratos Genomics (see, e.g., Kokoris et al., U.S. Pat. No. 7,939,259, “High Throughput Nucleic Acid Sequencing by Expansion”). In general terms, SBX uses this biochemical polymerization to transcribe the sequence of a DNA template onto a measurable polymer called an “Xpandomer”. The transcribed sequence is encoded along the Xpandomer backbone in high signal-to-noise reporters that are separated by about 10 nm and are designed for high-signal-to-noise, well-differentiated responses. These differences provide significant performance enhancements in sequence read efficiency and accuracy of Xpandomers relative to native DNA. A generalized overview of the SBX process is depicted in FIGS. 1A, 1B, 1C and 1D.

XNTPs are expandable, 5′ triphosphate modified nucleotide substrates compatible with template dependent enzymatic polymerization. A highly simplified XNTP is illustrated in FIG. 1A, which emphasizes the unique features of these nucleotide analogs: XNTP 100 has two distinct functional regions; namely, a selectively cleavable phosphoramidate bond 110, linking the 5′ α-phosphate 115 to the nucleobase 105, and a tether 120 that is attached within the nucleoside triphosphoramidate at positions that allow for controlled expansion by intra-nucleotide cleavage of the phosphoramidate bond. The tether of the XNTP is comprised of linker arm moieties 125A and 125B separated by the selectively cleavable phosphoramidate bond. Each linker attaches to one end of a reporter 130 via a linking group (LG), as disclosed in U.S. Pat. No. 8,324,360 to Kokoris et al., which is herein incorporated by reference in its entirety. XNTP 100 is illustrated in the “constrained configuration”, characteristic of the XNTP substrates and the daughter strand following polymerization. The constrained configuration of polymerized XNTPs is the precursor to the expanded configuration, as found in Xpandomer products. The transition from the constrained configuration to the expanded configuration occurs upon scission of the P—N bond of the phosphoramidate within the primary backbone of the daughter strand.

Synthesis of an Xpandomer is summarized in FIGS. 1B and 1C. During assembly, the monomeric XNTP substrates 145 (XATP, XCTP, XGTP and XTTP) are polymerized on the extendable terminus of a nascent daughter strand 150 by a process of template-directed polymerization using single-stranded template 140 as a guide. Generally, this process is initiated from a primer and proceeds in the 5′ to 3′ direction. Generally, a DNA polymerase or other polymerase is used to form the daughter strand, and conditions are selected so that a complimentary copy of the template strand is obtained. After the daughter strand is synthesized, the coupled tethers comprise the constrained Xpandomer that further comprises the daughter strand. Tethers in the daughter strand have the “constrained configuration” of the XNTP substrates. The constrained configuration of the tether is the precursor to the expanded configuration, as found the Xpandomer product.

As shown in FIG. 1C, the transition from the constrained configuration 160 to the expanded configuration 165 results from cleavage of the selectively cleavable phosphoramidate bonds (illustrated for simplicity by the unshaded ovals) within the primary backbone of the daughter strand. In this embodiment, the tethers comprise one or more reporters or reporter constructs, 130A, 130C, 130G, or 130T, specific for the nucleobase to which they are linked, thereby encoding the sequence information of the template. In this manner, the tethers provide a means to expand the length of the Xpandomer and lower the linear density of the sequence information of the parent strand.

FIG. 1D illustrates an Xpandomer 165 translocating through a nanopore 180, from the cis reservoir 175 to the trans reservoir 185. Upon passage through the nanopore, each of the reporters of the linearized Xpandomer (in this illustration, labeled “G”, “C” and “T”) generates a distinct and reproducible electronic signal (illustrated by superimposed trace 190), specific for the nucleobase to which it is linked.

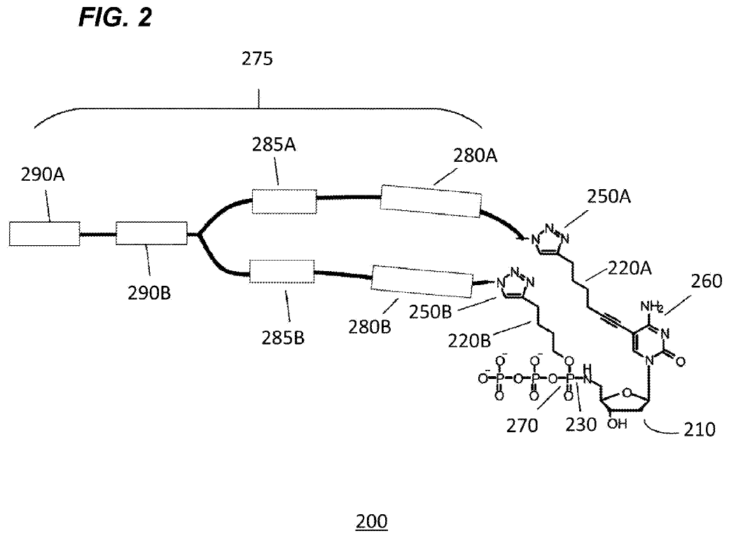

FIG. 2 depicts the generalized structure of an XNTP in more detail. XNTP 200 is comprised of nucleobase triphosphoramidate 210 with linker arm moieties 220A and 220B separated by selectively cleavable phosphoramidate bond 230. Tethers are joined to the nucleoside triphosphoramidate at linking groups 250A and 250B, wherein a first tether end is joined to the heterocycle 260 (represented here by cytosine, though the heterocycle may be any one of the four standard nucleobases, A, C, G, or T) and the second tether end is joined to the alpha phosphate 270 of the nucleobase backbone. The skilled artisan will appreciate that many suitable coupling chemistries known in the art may be used to form the final XNTP substrate product, for example, tether conjugation may be accomplished through a triazole linkage.

In this embodiment, tether 275 is comprised of several functional elements, including enhancers 280A and 280B, reporter codes 285A and 285B, and translation control elements (TCEs) 290A and 290B. Each of these features performs a unique function during translocation of the Xpandomer through a nanopore and generation of a unique and reproducible electronic signal. Tether 275 is designed for translocation control by hybridization (TCH). As depicted, the TCEs provide a region of hybridization which can be duplexed to a complementary oligomer (CO) and are positioned adjacent to the reporter codes. Different reporter codes are sized to block ion flow through a nanopore at different measurable levels. Specific reporter codes can be efficiently synthesized using phosphoramidite chemistry typically used for oligonucleotide synthesis. Reporters can be designed by selecting a sequence of specific phosphoramidites from commercially available libraries. Such libraries include but are not limited to polyethylene glycol with lengths of 1 to 12 or more ethylene glycol units, aliphatic with lengths of 1 to 12 or more carbon units, deoxyadenosine (A), deoxycytosine (C), deoxyguanodine (G), deoxythymine (T), abasic (Q). The duplexed TCEs associated with the reporter codes also contribute to the ion current blockage, thus the combination of the reporter code and the TCE can be referred to as a “reporter”. Following the reporter codes are the enhancers, which in one embodiment comprise spermine polymers.

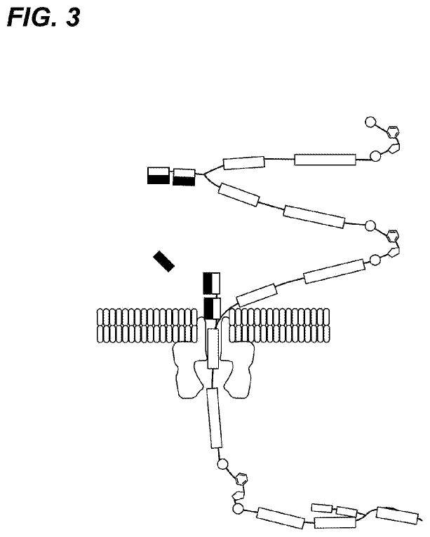

FIG. 3 shows one embodiment of a cleaved Xpandomer in the process of translocating an a-hemolysin nanopore. This biological nanopore is embedded into a lipid bilayer membrane which separates and electrically isolates two reservoirs of electrolytes. A typical electrolyte has 1 molar KCl buffered to a pH of 7.0. When a small voltage, typically 100 mV, is applied across the bilayer, the nanopore constricts the flow of ion current and is the primary resistance in the circuit. Xpandomer reporters are designed to give specific ion current blockage levels and sequence information can be read by measuring the sequence of ion current levels as the sequence of reporters translocate the nanopore.

The a-hemolysin nanopore is typically oriented so translocation occurs by entering the vestibule side and exiting the stem side. As shown in FIG. 3, the nanopore is oriented to capture the Xpandomer from the stem side first. This orientation is advantageous using the TCH method because it causes fewer blockage artifacts that occur when entering vestibule first. Unless indicated otherwise, stem side first will be the assumed translocation direction. As the Xpandomer translocates, a reporter enters the stem until its duplexed TCE stops at the stem entrance. The duplex is about 2.4 nm in diameter whereas the stem entrance is about 2.2 nm, so the reporter is held in the stem until the complimentary strand 395 of the duplex disassociates (releases) whereupon translocation proceeds to the next reporter. The free complementary strand is highly disfavored from entering the nanopore because the Xpandomer is still translocating and diffuses away from the pore.

In one embodiment, each member of a reporter code (following the duplex) is formed by an ordered choice of phosphoramidites that can be selected from many commercial libraries. Each constituent phosphoramidite contributes to the net ion resistance according to its position in the nanopore (located after the duplex stop), its displacement, its charge, its interaction with the nanopore, its chemical and thermal environment and other factors. The charge on each phosphoramidite is due, in part, to the phosphate ion which has a nominal charge of −1 but is effectively reduced by counterion shielding. The force pulling on the duplex is due to these effective charges along the reporter which are acted upon by the local electric fields. Since each reporter can have a different charge distribution, it can exert a different force on the duplex for a given applied voltage. The force transmitted along the reporter backbone also serves to stretch the reporter out to give a repeatable blocking response.

The Sequencing by Expansion (SBX) methodology developed by the inventors provides significant performance enhancements in sequence read efficiency and accuracy of Xpandomers relative to native DNA. However, initial transcription of the sequence of the natural DNA template onto the measurable Xpandomer relies on the ability of DNA polymerase to utilize XNTPs as substrates (the generalized structure of an XNTP is discussed herein with reference to FIG. 1A and FIG. 2). The inventors have found that most DNA polymerases do not efficiently polymerize XNTPs. However, the inclusion of a suitable additive, such as a PEM of the present disclosure improves the efficiency and accuracy of XNTP polymerization into Xpandomers. Thus, PEMs as disclosed herein may be used in the context of SBX methodology to enhance DNA polymerase primer extension reactions using XNTPs as substrates.

Examples of suitable concentrations for the components of the composition, when present, are as follows:
0.5-50 mM PEM (such as 1-50 mM, such as 2-50 mM, such as 3-50 mM, such as 4-50 mM, such as 5-50 mM),
0.02-0.2 μg/μl polymerase,
20-150 mM nucleotides or nucleotide analogs,
0.1-1 mM MnCl₂,
20-100 mM buffering agent,
100-200 mM salt,
0.05-0.5% (w/v) sugar,
0.05-0.5 mM polyphosphate,
140-300 mM imidazole,
50-200 mM pyrazole,
50-200 mM triazole,
2-8% (w/v) alkanediol,
2-10% (w/v) acetamide,
0.1-1% (w/v) glycerol
200-400 mM betaine,
10-30% (w/v) PEG,
3-10% (v/v) NMP,
0.5-1.5 mM BHA,
1-5% (v/v) DMSO,
0.5-2 mM urea,
0.02-1 μg/μl SSB.

In some embodiments, a primer extension reaction may include an aqueous composition consisting of the following components in water: 0.02-0.2 μg/μl polymerase, 0.1-1 mM MnCl2, 20-150 μM XNTPs, 20-100 mM buffer, 100-200 mM salt, 0.05-0.5% (w/v) sugar, 5-50 mM PEM, 0.05-0.5 mM polyphosphate, and optionally one or more selected from the group consisting of 140-300 mM imidazole, 2-8% (w/v) alkanediol, 2-10% (w/v) acetamide, 0.1-1% (w/v) glycerol, 200-400 mM betaine, 10-30% (w/v) PEG, 3-10% (v/v) NMP, 0.5-1.5 mM BHA, and 1-5% (v/v) DMSO, and further optionally 0.02-1 μg/μl SSB.

In some embodiments, a primer extension reaction may include an aqueous composition consisting of the following components in water: 0.02-0.2 μg/μl polymerase, 0.1-1 mM MnCl2, 20-150 μM XNTPs, 20-100 mM buffering agent, 100-200 mM salt, 0.05-0.5% (w/v) sugar, 5-50 mM PEM, 0.05-0.5 mM HMP, and optionally one or more selected from the group consisting of 140-300 mM imidazole, 200-400 mM betaine, 10-30% (w/v) PEG, 3-10% (v/v) NMP, 0.5-1.5 mM BHA, and 1-5% (v/v) DMSO, and further optionally 0.02-1 μg/μl SSB.

In some embodiments, a primer extension reaction may include an aqueous composition consisting of the following components in water: 0.02-0.2 μg/μl polymerase, 0.1-1 mM MnCl2, 20-150 μM XNTPs, 20-100 mM buffering agent, 100-200 mM salt, 0.05-0.5% (w/v) sugar, 5-50 mM PEM, 0.05-0.5 mM HMP, and optionally one or more selected from the group consisting of 140-300 mM imidazole, 10-30% (w/v) PEG, and 2-8% (w/v) alkanediol, and further optionally 0.02-1 μg/μl SSB.

In some embodiments, a primer extension reaction may include an aqueous composition consisting of the following components in water: 0.02-0.2 μg/μl polymerase, 0.1-1 mM MnCl2, 20-150 μM XNTPs, 20-100 mM buffering agent, 100-200 mM salt, 0.05-0.5% (w/v) sugar, 5-50 mM PEM, 0.05-0.5 mM HMP, and optionally one or more selected from the group consisting of 140-300 mM imidazole, 10-30% (w/v) PEG, and 2-10% (w/v) acetamide, and further optionally 0.02-1 μg/μl SSB.

In some embodiments, a primer extension reaction may include an aqueous composition consisting of the following components in water: 0.02-0.2 μg/μl polymerase, 0.1-1 mM MnCl2, 20-150 μM XNTPs, 20-100 mM buffering agent, 100-200 mM salt, 0.05-0.5% (w/v) sugar, 5-50 mM PEM, 0.05-0.5 mM HMP, and optionally one or more selected from the group consisting of 140-300 mM imidazole, 10-30% (w/v) PEG, 0.1-1% (w/v) glycerol, 2-8% (w/v) alkanediol, and 2-10% (w/v) acetamide, and further optionally 0.02-1 μg/μl SSB.

Typically, the primer extension reaction may also include a primer and a template, such as an oligonucleotide template.

A representative primer extension reaction may include the following reagents: 2 pmol primer, 2.2 pmol 45mer oligonucleotide template, 50 pmol of each XNTP (XATP, XCTP, XGTP, and XTTP), 50 mM Tris HCl, pH 6.79, 200 mM NaCl, 20% PEG, 5% NMS, 0.5 nmol polyphosphate 60.19, 0.3 mM MnCl₂, and 0.6 μg of purified recombinant DNA polymerase protein. PEMs are added to this mixture at a concentration typically in the micro to millimolar range. Reactions may also include additional additives, such as single-strand binding protein (SSB), urea, and NMS. Reactions are run for 1 hour at 23 °C. Reaction products (i.e., constrained Xpandomers) are treated to cleave the phosphoramidate bonds, thereby to generating linearized Xpandomers. Reaction products are analyzed using gel electrophoresis on 4-12% acrylamide gels to resolve and visualize Xpandomer products of different lengths.

Thus, in one embodiment, the present disclose provides an aqueous (water containing) composition comprising a PEM and a buffer, particularly a buffer suitable for conducting a DNA polymerization reaction, where Tris HCl is an exemplary buffer of this type. In one embodiment, the present disclosure provides a composition comprising a PEM and a DNA polymerase protein. In one embodiment, the present disclosure provides a composition comprising a PEM and a polynucleotide, e.g., a 20-90 mer, 20-60 mer, 30-90 mer, or a 30-60 mer, oligonucleotide. In one embodiment, the present disclosure provides a composition that comprises each of these components, i.e., an aqueous composition comprising a PEM, a buffer, a DNA polymerase protein, and a polynucleotide.

To investigate the accuracy of enhancement of XNTP polymerization, primer extension products may be sequenced using the SBX protocol. Briefly, the constrained Xpandomer products of XNTP polymerization are cleaved to generate linearized Xpandomers. This is accomplished by first quenching the extension reaction with a solution containing 100 mM EDTA, 2 mM THPTA, and 2% Tween-20. Then the sample is subjected to amine modification with a solution of 1 M NaHCO₃ and 1 M succinic anhydride in DMF. Cleavage of the phosphoramidate bonds is carried out with 37% HCl and linearized Xpandomers are purified with QIAquick columns (QIAGEN, Inc.).

For sequencing, protein nanopores are prepared by inserting a-hemolysin into a DPhPE/hexadecane bilayer member in buffer B1, containing 2 M NH₄Cl and 100 mM HEPES, pH 7.4. The cis well is perfused with buffer B2, containing 0.4 M NH₄Cl, 0.6 M GuCl, and 100 mM HEPES, pH 7.4. The Xpandomer sample is heated to 70 °C for 2 minutes, cooled completely, then a 2 μL sample is added to the cis well. A voltage pulse of 90 mV/390 mV/10 μs is then applied and data is acquired via Labview acquisition software.

Sequence data is analyzed by histogram display of the population of sequence reads from a single SBX reaction. The analysis software aligns each sequence read to the sequence of the template and trims the extent of the sequence at the end of the reads that does not align with the correct template sequence.

In one embodiment the present disclosure provides a method of increasing the accuracy of enhancement of XNTP polymerization, where the method comprises adding a PEM as disclosed herein to the DNA polymerization reaction as described above.

In some embodiments, the present disclosure provides a kit, where the kit may be used in a method as described herein. The kit will include at least one compound of the present disclosure, and one or more of a) a molecular crowding agent, b) an aqueous buffer, c) a protein such as a polymerase, d) a polynucleotide which may function, for example, as a primer, and/or a polynucleotide which may function, for example, as a template.

For example, in some embodiments the present disclosure provides a kit for sequencing a nucleic acid template. The kit includes at least one compound of the present disclosure and a mixture of nucleotide analogs. The compound of the present disclosure may be used to increases the number and accuracy of nucleotide analogs incorporated into a daughter strand during a template-dependent polymerization reaction relative to an identical polymerization reaction absent the at least one compound of the present disclosure. Optionally, the mixture of nucleotide analogs comprises nucleoside triphosphoramidates, wherein each of the nucleoside triphosphoramidates comprises a nucleobase selected from the group consisting of adenine, guanine, thymine, and cytosine and a polymeric tether moiety, wherein a first end of the polymeric tether moiety is attached to the nucleobase and a second end of the polymeric ether moiety is attached to the alpha phosphate of the nucleoside triphosphoramidate to provide for expansion of the nucleotide analogs by cleavage of the phosphoramidate bond. Optionally, the mixture of nucleotide analogs comprises nucleotide analogs comprising a detectable label, where the detectable label is an optically detectable label selected from the group consisting of luminescent, chemiluminescent, fluorescent, fluorogenic, chromophoric or chromogenic labels. Optionally, the kit includes an aqueous buffer which includes one or more components selected from MnCl₂, a buffer, a salt, a sugar, a single-strand binding protein (SSB), imidazole, pyrazole, triazole, betaine, a molecular crowding agent, dimethyl sulfoxide (DMSO), an alkanediol, glycerol, N-methyl-2-pyrrolidone (NMP), acetamide, butylated hydroxyanisole (BHA), a polyphosphate, and/or urea. Optionally, the kit includes a single-strand binding protein. Optionally, the kit includes urea. Optionally, the kit includes two or more of these components, e.g., 3, or 4, or all of the named components.

Compounds may be prepared by methods known to one of ordinary skill in the art, where such methods may be identified through various reference books and databases. Suitable reference books and treatise that detail the synthesis of reactants useful in the preparation of compounds of the present disclosure, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure,” 4th Ed., Wiley-Interscience, New York, 1992. Additional suitable reference books and treatise that detail the synthesis of reactants useful in the preparation of compounds of the present disclosure, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

### EXAMPLES

Compounds as shown in Table 1 were synthesized, such as in accordance with the general Examples disclosed herein.

Materials and Methods. 4-Azido salicylic acid and 2,6-dibromo-4-pyridine carboxylic acid were from Toronto Research Chemicals, Inc. (Toronto, ON, Canada). 4-azido-2-(trifluoromethyl)benzoic acid, 3-Amino-5-hydroxybenzoic acid and 4-aminoisophthalic acid were from Matrix Scientific (Columbia, SC, USA). 3-amino-6-(trifluoromethyl)benzoic acid hydrochloride, 1-(4-aminophenyl)-2,2,2-trifluoroethan-1-one, methyl glycylglycinate hydrochloride, 3,3,3-trifluoropropan-1-amine and diethyl 3-aminopropan-1-ylphosphonate were from Enamine LLC (Monmouth Junction, NJ, USA). Tris[(1-benzyl-1H-1,2,3-triazol-4-yl)methyl]amine (TBTA), 0-(7-Azabenzotriazol-1-yl)-N,N,N′,N′-tetramethyluronium hexafluoro-phosphate (HATU), 1,3-diethynylbenzene, 2,6-diethynylpyridine, 3,5-diethynylpyridine, 3,6-diethynylcarbazole, 4-azidobenzoic acid, cyclopropyl amine, 6-amino-2-naphthoic acid, 4-aminophthalic acid, 4-amino-3-hydroxybenzoic acid, 4-amino-2-methylbenzoic acid, 4-amino-2,3,5,6-tetrafluorobenzoic acid, 4-amino-2-nitrobenzoic acid, n-(2-aminoethyl)acetamide, 4-(aminophenyl)phosphonic acid, 2,6-dichloro-9H-purine, Dimethyl aspartate hydrochloride, 4-amino-2-(trifluoromethyl)benzonitrile, 3,5-diaminobenzoic acid and 2,5-dibromofuran were from TCI America (Portland, OR, USA). 4-methoxy-2,6-dibromopyridine,4-nitro-2,6-dibromopyridine and 2,6-dibromo-4-pyridine carboxylic acid, Methyl glycinate hydrochloride were from Chem-Impex International, Inc. (Wood Dale, IL). Tetrakis(triphenylphosphine)palladium (0), ethynyltrimethylsilane, DMSO, DMF, MeOH, EtOAc, sodium ascorbate, copper sulfate, diisorpropylamine, EDTA, morpholine, diethylamine, ammonium hydroxide, ethylamine, ethanol, azetidine, n-ethyl-n-ethylamine, methyl 2,6-dichloropyridine-4-carboxylate, ethyl 2,6-dibromopyridine-4-carboxylate, 4-methyl-2,6-dichloropyridine,2-chloro-4-cyanopyridine, 4-amino-2-(trifluoromethyl)benzoic acid, 2-bromo-4-cyanopyridine, methylazido acetate, 4-azidoaniline hydrochloride, 4-methoxyphenyl azid4-amino-2-fluorobenzoic acid, n-butylamine, 3-amino-5-(trifluoromethyl)benzoic acid, 3-(4-aminophenyl)propionic acid, 4-(4-aminophenyl)-butyric acid, 4-amino-2-methoxybenzoic acid, 2-amino-3-(trifluoromethyl)benzoic acid, aminoethanol, 1,4-diaminobutane and 2,5-dibromothiophene were from Sigma-Aldrich Corp. (St. Louis, MO, USA). TLC and flash chromatography solvents were from Sigma-Aldrich or Thermo Fisher Scientific Inc. (Waltham, MA, USA).

Flash chromatography was performed on a Reveleris Prep Purification System from Buchi Corp. (New Castle, DE). The system was fitted with a hand packed column (2.3 cm diameter X 8 cm height) filled with C₁₈ Spherical Silica Gel (Cat. No. 76646-01) from Sorbent Technologies, Inc (Norcross, GA) and sealed with polypropylene frits. Samples of 1 to 1.5 mL were loaded directly on the head of the column. Mobile phases were water (A) and acetonitrile (B). A gradient of 0 to 2% B in 2 minutes followed by 2 to 100% B in 20 minutes at a flow of 28 ml/min. UV was monitored at 220 nm, 260 nm and 280 nm. Fractions were collected at UV threshold of 0.1 AU. Thin layer chromatography was performed with aluminum backed TLC Silica Gel 60 F254 (Cat. No. 1.05534.0001) from EMD Millipore Corp. (Billireca, MA, USA). ESI Mass Spec was performed by Numega Resonance Lab (San Diego, CA, USA) using a Perkin Elmer PE-SCLEX API-150 mass spectrometer in positive and negative mode.

TABLE 1

| Compound | Chemical Name | Structure |
|---|---|---|
| 1 | 4,4′-(pyridine-2,6-diylbis(1H-1,2,3-triazole- 4,1-diyl))bis(2-hydroxybenzoic acid) | 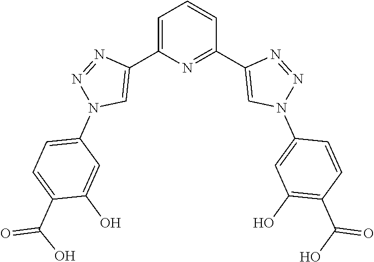 |
| 2 | 4,4′-(pyridine-3,5-diylbis(1H-1,2,3-triazole- 4,1-diyl))bis(2-hydroxybenzoic acid) | 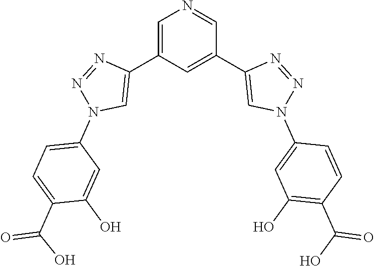 |
| 3 | 4,4′-(1,3-phenylenebis(1H-1,2,3-triazole-4,1- diyl))bis(2-hydroxybenzoic acid) | 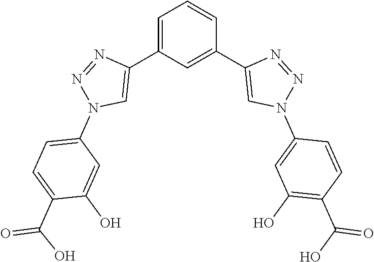 |
| 4 | 4,4′-((9H-carbazole-3,6-diyl)bis(1H-1,2,3- triazole-4,1-diyl))bis(2-hydroxybenzoic acid) | 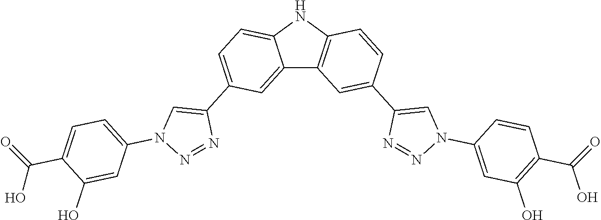 [SMILES: O=C(O)c1ccc(-n2cc(-c3ccc4[nH]c5ccc(-c6cn(-c7ccc(C(=O)O)c(O)c7)nn6)cc5c4c3)nn2)cc1O] |
| 5 | 4,4′-((9H-carbazole-3,6-diyl)bis(1H-1,2,3- triazole-4,1-diyl))dianiline | 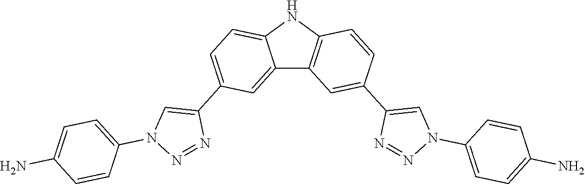 [SMILES: Nc1ccc(-n2cc(-c3ccc4[nH]c5ccc(-c6cn(-c7ccc(N)cc7)nn6)cc5c4c3)nn2)cc1] |
| 6 | 4,4′-((9H-carbazole-3,6-diyl)bis(1H-1,2,3- triazole-4,1-diyl))dibenzoic acid | 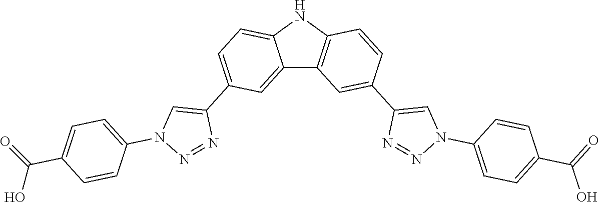 [SMILES: O=C(O)c1ccc(-n2cc(-c3ccc4[nH]c5ccc(-c6cn(-c7ccc(C(=O)O)cc7)nn6)cc5c4c3)nn2)cc1] |
| 7 | 3,6-bis(1-(4-methoxyphenyl)-1H-1,2,3- triazol-4-yl)-9H-carbazole | 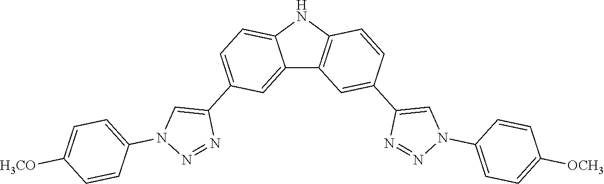 [SMILES: COc1ccc(-n2cc(-c3ccc4[nH]c5ccc(-c6cn(-c7ccc(OC)cc7)nn6)cc5c4c3)nn2)cc1] |
| 8 | dimethyl 2,2′-((9H-carbazole-3,6- diyl)bis(1H-1,2,3-triazole-4,1-diyl))diacetate | 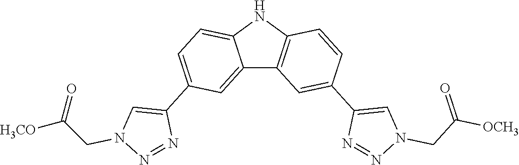 [SMILES: COC(=O)Cn1cc(-c2ccc3[nH]c4ccc(-c5cn(CC(=O)OC)nn5)cc4c3c2)nn1] |
| 9 | 4,4′-((4-methoxypyridine-2,6-diyl)bis(1H- 1,2,3-triazole-4,1-diyl))bis(2-hydroxybenzoic acid) | 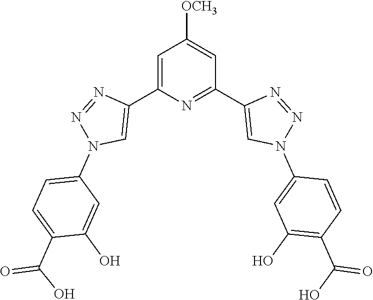 [SMILES: COc1cc(-c2cn(-c3ccc(C(=O)O)c(O)c3)nn2)nc(-c2cn(-c3ccc(C(=O)O)c(O)c3)nn2)c1] |
| 10 | 4,4′-((4-carboxypyridine-2,6-diyl)bis(1H- 1,2,3-triazole-4,1-diyl))bis(2-hydroxybenzoic acid) | 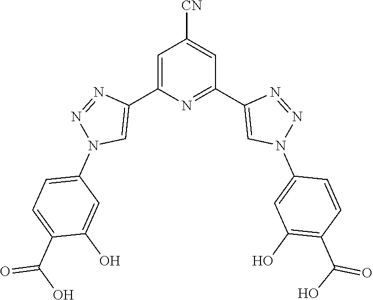 [SMILES: N#Cc1cc(-c2cn(-c3ccc(C(=O)O)c(O)c3)nn2)nc(-c2cn(-c3ccc(C(=O)O)c(O)c3)nn2)c1] |
| 11 | 4,4′-((4-nitropyridine-2,6-diyl)bis(1H-1,2,3- triazole-4,1-diyl))bis(2-hydroxybenzoic acid) | 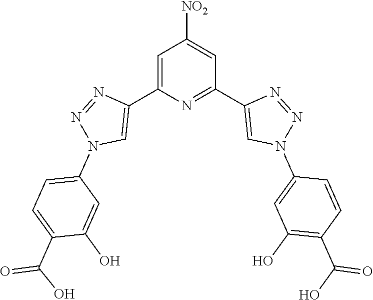 [SMILES: O=C(O)c1ccc(-n2cc(-c3cc([N+](=O)[O-])cc(-c4cn(-c5ccc(C(=O)O)c(O)c5)nn4)n3)nn2)cc1O] |
| 12 | 5,5′-((4-cyanopyridine-2,6-diyl)bis(1H-1,2,3- triazole-4,1-diyl))bis(2-hydroxybenzoic acid) | 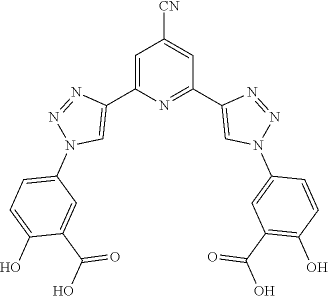 |
| 13 | 4,4′-((4-methylpyridine-2,6-diyl)bis(1H- 1,2,3-triazole-4,1-diyl))bis(2-hydroxybenzoic acid) | 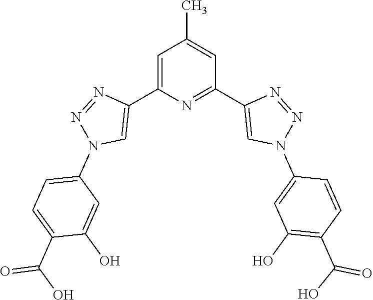 |
| 14 | 4,4′-((4-(ethoxycarbonyl)pyridine-2,6- diyl)bis(1H-1,2,3-triazole-4,1-diyl))bis(2- hydroxybenzoic acid) | 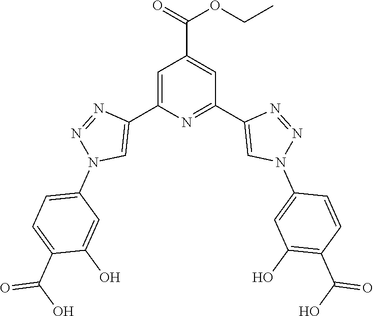 [SMILES: CCOC(=O)c1cc(-c2cn(-c3ccc(C(=O)O)c(O)c3)nn2)nc(-c2cn(-c3ccc(C(=O)O)c(O)c3)nn2)c1] |
| 15 | 5,5′-((4-(ethoxycarbonyl)pyridine-2,6- diyl)bis(1H-1,2,3-triazole-4,1-diyl))bis(2- hydroxybenzoic acid) | 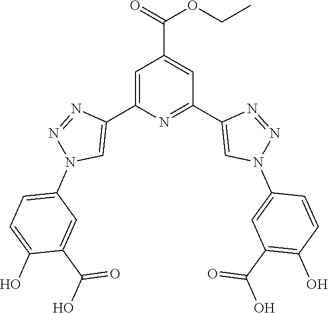 |
| 16 | 4,4′-((4-(methoxycarbonyl)pyridine-2,6- diyl)bis(1H-1,2,3-triazole-4,1-diyl))bis(2- hydroxybenzoic acid) | 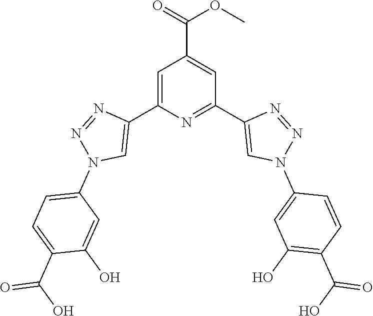 [SMILES: COC(=O)c1cc(-c2cn(-c3ccc(C(=O)O)c(O)c3)nn2)nc(-c2cn(-c3ccc(C(=O)O)c(O)c3)nn2)c1] |
| 17 | 4,4′-((4-(ethylcarbamoyl)pyridine-2,6- diyl)bis(1H-1,2,3-triazole-4,1-diyl))bis(2- hydroxybenzoic acid) | 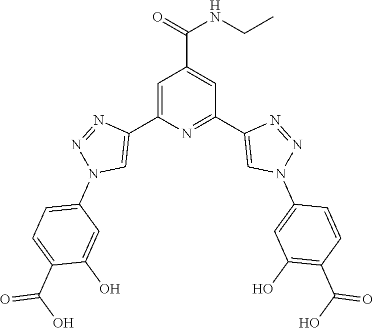 |
| 18 | 4,4′-((4-(methylcarbamoyl)pyridine-2,6- diyl)bis(1H-1,2,3-triazole-4,1-diyl))bis(2- hydroxybenzoic acid) | 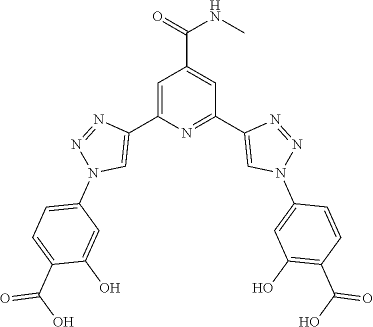 |
| 19 | 4,4′-((4-carbamoylpyridine-2,6-diyl)bis(1H- 1,2,3-triazole-4,1-diyl))bis(2-hydroxybenzoic acid) | 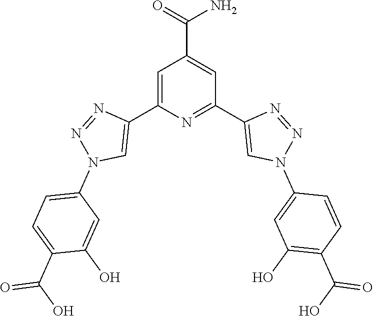 [SMILES: NC(=O)c1cc(-c2cn(-c3ccc(C(=O)O)c(O)c3)nn2)nc(-c2cn(-c3ccc(C(=O)O)c(O)c3)nn2)c1] |
| 20 | 4,4′-(pyrazine-2,6-diylbis(1H-1,2,3-triazole- 4,1-diyl))bis(2-hydroxybenzoic acid) | 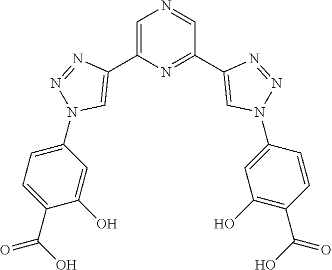 [SMILES: O=C(O)c1ccc(-n2cc(-c3cncc(-c4cn(-c5ccc(C(=O)O)c(O)c5)nn4)n3)nn2)cc1O] |
| 21 | 4,4′-(1,4-phenylenebis(1H-1,2,3-triazole-4,1- diyl))bis(2-hydroxybenzoic acid) | 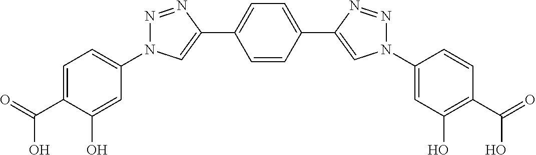 [SMILES: O=C(O)c1ccc(-n2cc(-c3ccc(-c4cn(-c5ccc(C(=O)O)c(O)c5)nn4)cc3)nn2)cc1O] |
| 22 | 4,4′-(1,3-phenylenebis(1H-1,2,3-triazole-4,1- diyl))dianiline | 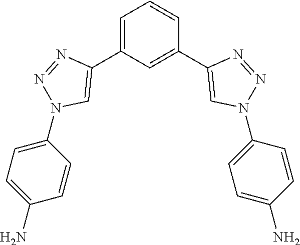 [SMILES: Nc1ccc(-n2cc(-c3cccc(-c4cn(-c5ccc(N)cc5)nn4)c3)nn2)cc1] |
| 23 | 4,4′-(1,3-phenylenebis(1H-1,2,3-triazole-4,1- diyl))dibenzoic acid | 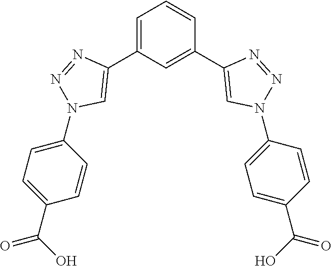 [SMILES: O=C(O)c1ccc(-n2cc(-c3cccc(-c4cn(-c5ccc(C(=O)O)cc5)nn4)c3)nn2)cc1] |
| 24 | 1,3-bis(1-(4-methoxyphenyl)-1H-1,2,3- triazol-4-yl)benzene | 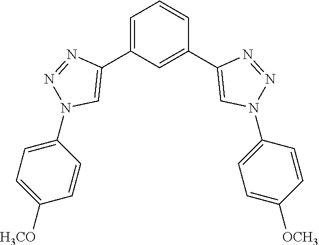 [SMILES: COc1ccc(-n2cc(-c3cccc(-c4cn(-c5ccc(OC)cc5)nn4)c3)nn2)cc1] |
| 25 | 4,4′-(pyridine-2,6-diylbis(1H-1,2,3-triazole- 4,1-diyl))dianiline | 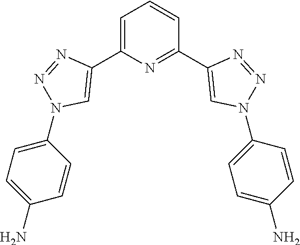 [SMILES: Nc1ccc(-n2cc(-c3cccc(-c4cn(-c5ccc(N)cc5)nn4)n3)nn2)cc1] |
| 26 | 4,4′-(pyridine-2,6-diylbis(1H-1,2,3-triazole- 4,1-diyl))dibenzoic acid | 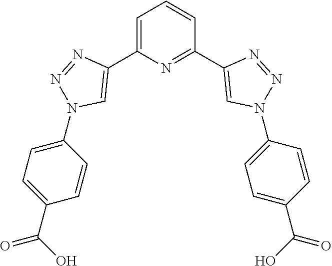 [SMILES: O=C(O)c1ccc(-n2cc(-c3cccc(-c4cn(-c5ccc(C(=O)O)cc5)nn4)n3)nn2)cc1] |
| 27 | 2,6-bis(1-(4-methoxyphenyl)-1H-1,2,3- triazol-4-yl)pyridine | 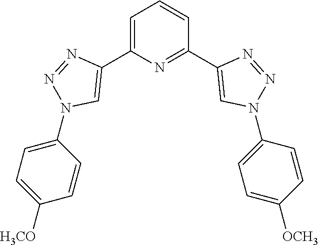 [SMILES: COc1ccc(-n2cc(-c3cccc(-c4cn(-c5ccc(OC)cc5)nn4)n3)nn2)cc1] |
| 28 | 2-[tert-Butyl(oxycarbonylamino)]-5-{4-[6- (1-{4-[tert-butyl(oxycarbonylamino)]-4- carboxybutyl}-1H-1,2,3-triazol-4-yl)-2- pyridyl]-1H-1,2,3-triazol-1-yl} valeric acid | 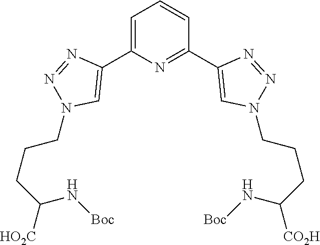 [SMILES: CC(C)(C)OC(=O)NC(CCCn1cc(-c2cccc(-c3cn(CCCC(NC(=O)OC(C)(C)C)C(=O)O)nn3)n2)nn1)C(=O)O] |
| 29 | 4-(4-(3-(1-(4-methoxyphenyl)-1H-1,2,3- triazol-4-yl)phenyl)-1H-1,2,3-triazol-1- yl)benzoic acid | 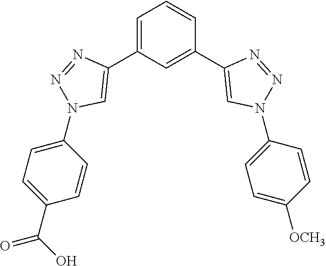 [SMILES: COc1ccc(-n2cc(-c3cccc(-c4cn(-c5ccc(C(=O)O)cc5)nn4)c3)nn2)cc1] |
| 30 | 4,4′-((3,5-dimethylpyridine-2,6-diyl)bis(1H- 1,2,3-triazole-4,1-diyl))bis(2-hydroxybenzoic acid) | 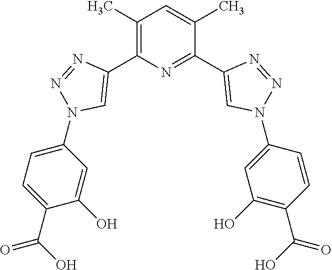 [SMILES: Cc1cc(C)c(-c2cn(-c3ccc(C(=O)O)c(O)c3)nn2)nc1-c1cn(-c2ccc(C(=O)O)c(O)c2)nn1] |
| 31 | 4,4′-((pyridine-2,6-diyl)bis(5-iodo-1H-1,2,3- triazole-4,1-diyl))bis(2-hydroxybenzoic acid) | 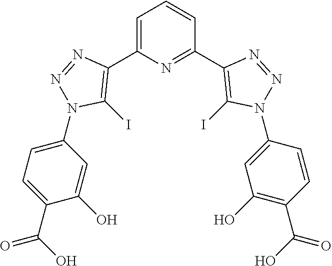 [SMILES: O=C(O)c1ccc(-n2nnc(-c3cccc(-c4nnn(-c5ccc(C(=O)O)c(O)c5)c4I)n3)c2I)cc1O] |
| 32 | 4,4′-((4-acetamidopyridine-2,6-diyl)bis(1H- 1,2,3-triazole-4,1-diyl))bis(2-hydroxybenzoic acid) | 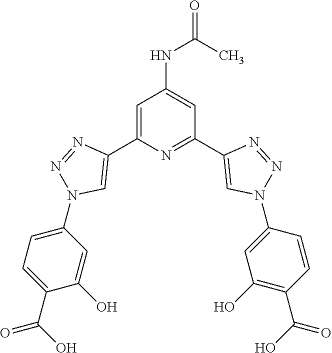 [SMILES: CC(=O)Nc1cc(-c2cn(-c3ccc(C(=O)O)c(O)c3)nn2)nc(-c2cn(-c3ccc(C(=O)O)c(O)c3)nn2)c1] |
| 33 | 4,4′-((9-acetyl-9H-carbazole-3,6-diyl)bis(1H- 1,2,3-triazole-4,1-diyl))bis(2-hydroxybenzoic acid) | 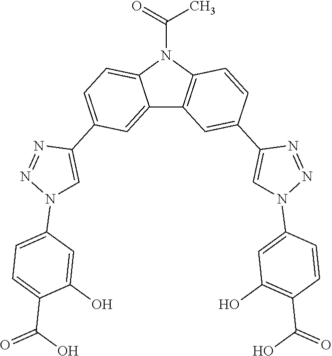 [SMILES: CC(=O)n1c2ccc(-c3cn(-c4ccc(C(=O)O)c(O)c4)nn3)cc2c2cc(-c3cn(-c4ccc(C(=O)O)c(O)c4)nn3)ccc21] |
| 34 | 4,4′-(pyridine-2,6-diylbis(1H-1,2,3-triazole- 4,1-diyl))bis(N,2-dihydroxybenzamide) | 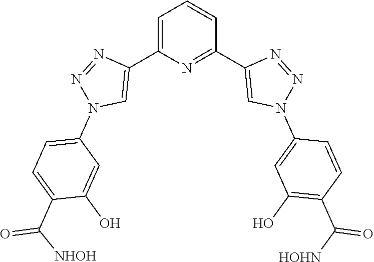 |
| 35 | 4,4′-(pyridine-2,6-diylbis(1H-1,2,3-triazole- 4,1-diyl))bis(2-hydroxybenzamide) | 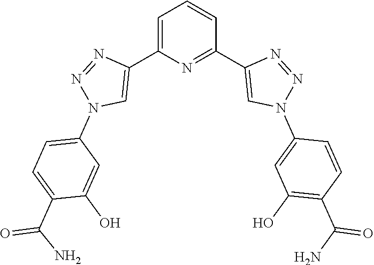 |
| 36 | 4,4′-((4-carboxypyridine-2,6-diyl)bis(1H- 1,2,3-triazole-4,1-diyl))bis(2-hydroxybenzoic acid) | 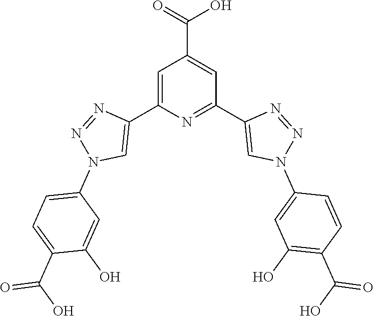 [SMILES: O=C(O)c1cc(-c2cn(-c3ccc(C(=O)O)c(O)c3)nn2)nc(-c2cn(-c3ccc(C(=O)O)c(O)c3)nn2)c1] |
| 37 | 4,4′-((1,10-phenanthroline-2,9-diyl)bis(1H- 1,2,3-triazole-4,1-diyl))bis(2-hydroxybenzoic acid) | 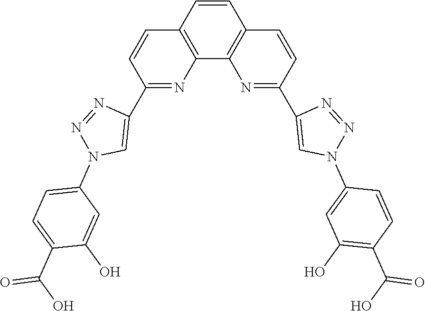 |
| 38 | 4,4′-((4-(trifluoromethyl)pyridine-2,6- diyl)bis(1H-1,2,3-triazole-4,1-diyl))bis(2- hydroxybenzoic acid) | 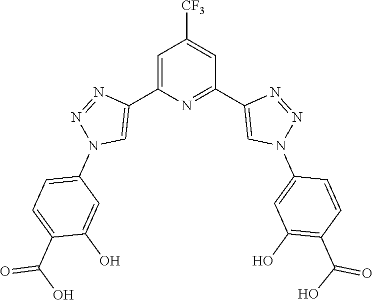 |
| 39 | 4,4′-((3-cyanopyridine-2,6-diyl)bis(1H-1,2,3- triazole-4,1-diyl))bis(2-hydroxybenzoic acid) | 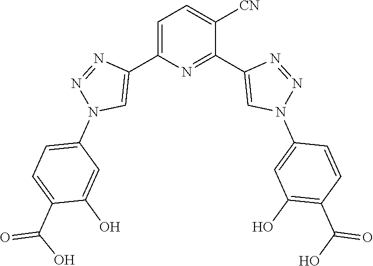 |
| 40 | 4,4′-((3-nitropyridine-2,6-diyl)bis(1H-1,2,3- triazole-4,1-diyl))bis(2-hydroxybenzoic acid) | 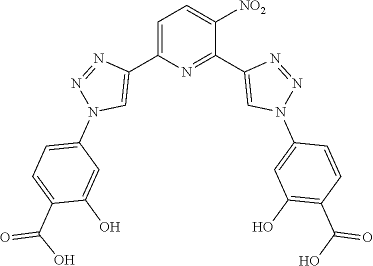 |
| 41 | 3,3′-((4-cyanopyridine-2,6-diyl)bis(1H-1,2,3- triazole-4,1-diyl))bis(2-hydroxybenzoic acid) | 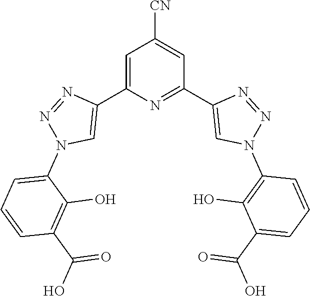 |
| 42 | 4,4′-((4-(tert-butoxycarbonyl)pyridine-2,6- diyl)bis(1H-1,2,3-triazole-4,1-diyl))bis(2- hydroxybenzoic acid) | 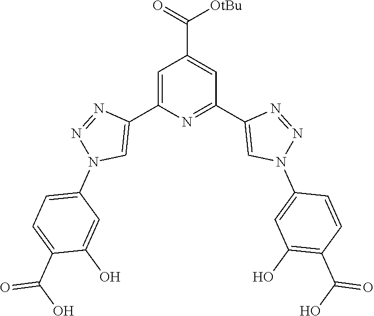 [SMILES: CC(C)(C)OC(=O)c1cc(-c2cn(-c3ccc(C(=O)O)c(O)c3)nn2)nc(-c2cn(-c3ccc(C(=O)O)c(O)c3)nn2)c1] |
| 43 | 4-(4-(4-cyanopyridin-2-yl)-1H-1,2,3-triazol- 1-yl)-2-hydroxybenzoic acid | 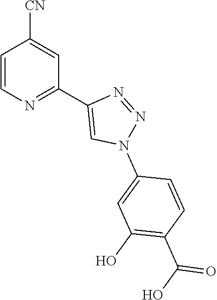 [SMILES: N#Cc1ccnc(-c2cn(-c3ccc(C(=O)O)c(O)c3)nn2)c1] |
| 44 | 5-(4-(6-(4-(3-carboxy-4-hydroxy-5- methylphenyl)-1H-1,2,3-triazol-1-yl)-4- (methoxycarbonyl)pyridin-2-yl)-1H-1,2,3- triazol-1-yl)-2-hydroxy-3-methylbenzoic acid | 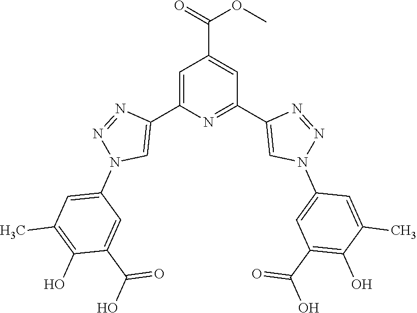 |
| 45 | 4,4′-((4-(dimethylcarbamoyl)pyridine-2,6- diyl)bis(1H-1,2,3-triazole-4,1-diyl))bis(2- hydroxybenzoic acid) | 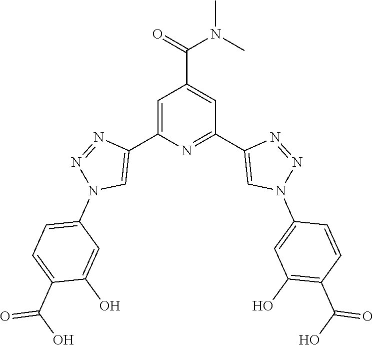 |
| 46 | 4,4′-((4-(cyclopropylcarbamoyl)pyridine-2,6- diyl)bis(1H-1,2,3-triazole-4,1-diyl))bis(2- hydroxybenzoic acid) | 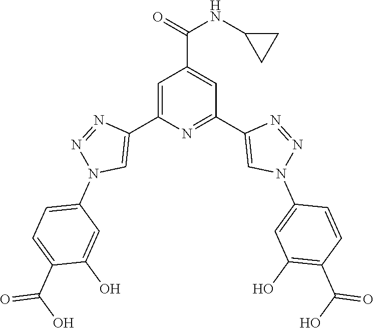 [SMILES: O=C(NC1CC1)c1cc(-c2cn(-c3ccc(C(=O)O)c(O)c3)nn2)nc(-c2cn(-c3ccc(C(=O)O)c(O)c3)nn2)c1] |
| 47 | 4,4′-((4-(but-3-yn-1-ylcarbamoyl)pyridine- 2,6-diyl)bis(1H-1,2,3-triazole-4,1-diyl))bis(2- hydroxybenzoic acid) | 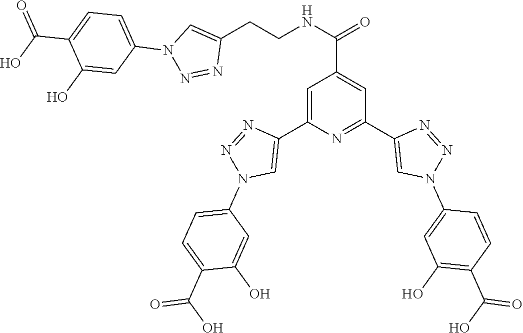 [SMILES: O=C(NCCc1cn(-c2ccc(C(=O)O)c(O)c2)nn1)c1cc(-c2cn(-c3ccc(C(=O)O)c(O)c3)nn2)nc(-c2cn(-c3ccc(C(=O)O)c(O)c3)nn2)c1] |
| 48 | 4,4′-((4-(butylcarbamoyl)pyridine-2,6- diyl)bis(1H-1,2,3-triazole-4,1-diyl))bis(2- hydroxybenzoic acid) | 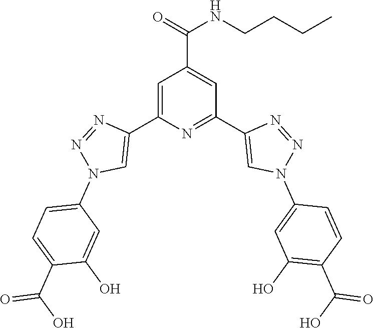 |
| 49 | 4,4′-((4-(diethylcarbamoyl)pyridine-2,6- diyl)bis(1H-1,2,3-triazole-4,1-diyl))bis(2- hydroxybenzoic acid) | 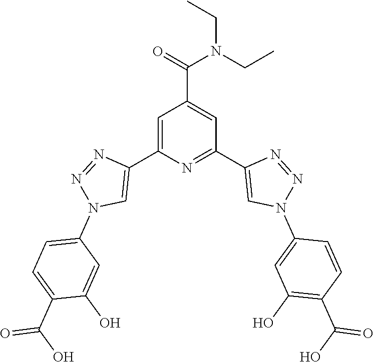 |
| 50 | 4,4′-((4-(tert-butylcarbamoyl)pyridine-2,6- diyl)bis(1H-1,2,3-triazole-4,1-diyl))bis(2- hydroxybenzoic acid) | 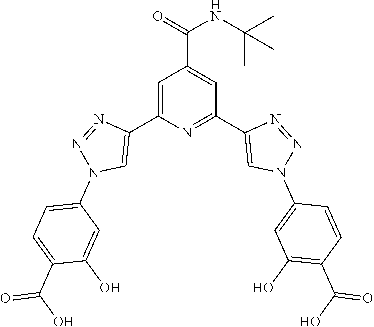 |
| 51 | 4,4′-((4-(morpholine-4-carbonyl)pyridine- 2,6-diyl)bis(1H-1,2,3-triazole-4,1-diyl))bis(2- hydroxybenzoic acid) | 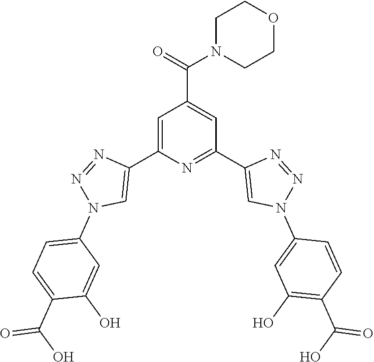 |
| 52 | 4,4′-((4-(propylcarbamoyl)pyridine-2,6- diyl)bis(1H-1,2,3-triazole-4,1-diyl))bis(2- hydroxybenzoic acid) | 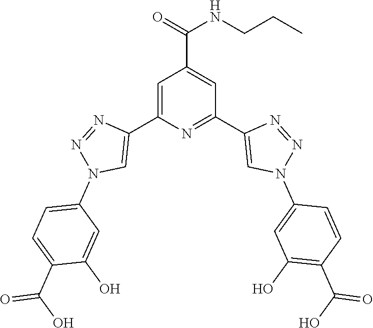 |
| 53 | 4,4′-((4-(phenylcarbamoyl)pyridine-2,6- diyl)bis(1H-1,2,3-triazole-4,1-diyl))bis(2- hydroxybenzoic acid) | 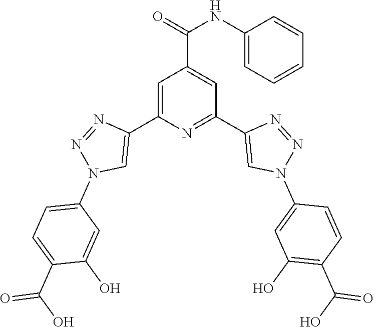 |
| 54 | 4,4′-((4-((2- acetamidoethyl)carbamoyl)pyridine-2,6- diyl)bis(1H-1,2,3-triazole-4,1-diyl))bis(2- hydroxybenzoic acid) | 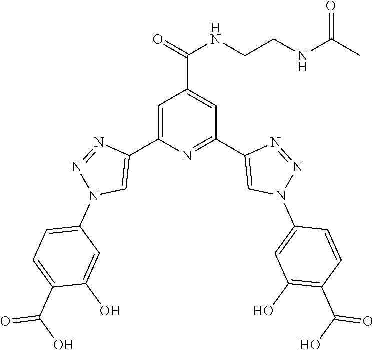 |
| 55 | 4,4′-((4-(4-cyclopropylpiperazine-1- carbonyl)pyridine-2,6-diyl)bis(1H-1,2,3- triazole-4,1-diyl))bis(2-hydroxybenzoic acid) | 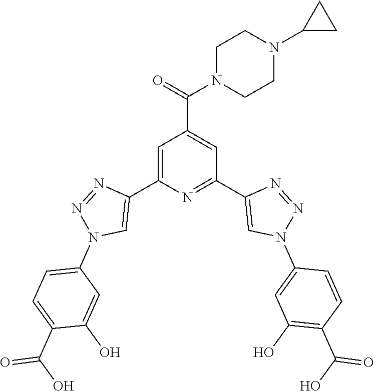 [SMILES: O=C(O)c1ccc(-n2cc(-c3cc(C(=O)N4CCN(C5CC5)CC4)cc(-c4cn(-c5ccc(C(=O)O)c(O)c5)nn4)n3)nn2)cc1O] |
| 56 | 4,4′-((4-(carbamimidoylcarbamoyl)pyridine- 2,6-diyl)bis(1H-1,2,3-triazole-4,1-diyl))bis(2- hydroxybenzoic acid) | 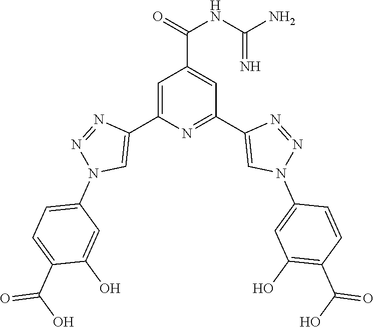 |
| 57 | 4,4′-((4-(piperidine-1-carbonyl)pyridine-2,6- diyl)bis(1H-1,2,3-triazole-4,1-diyl))bis(2- hydroxybenzoic acid) | 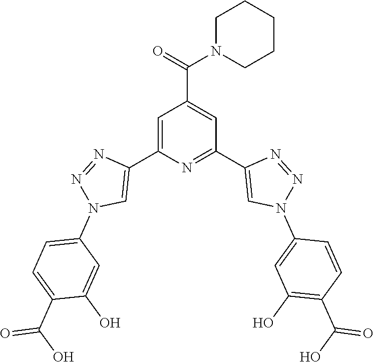 |
| 58 | 4,4′-((4-(cyclobutylcarbamoyl)pyridine-2,6- diyl)bis(1H-1,2,3-triazole-4,1-diyl))bis(2- hydroxybenzoic acid) | 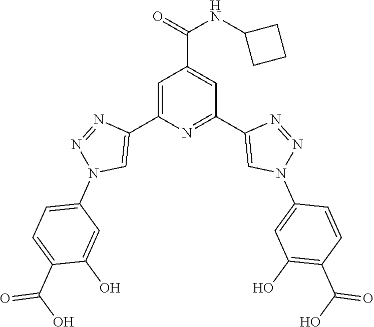 |
| 59 | 4,4′-((1,10-phenanthroline-3,8-diyl)bis(1H- 1,2,3-triazole-4,1-diyl))bis(2-hydroxybenzoic acid) | 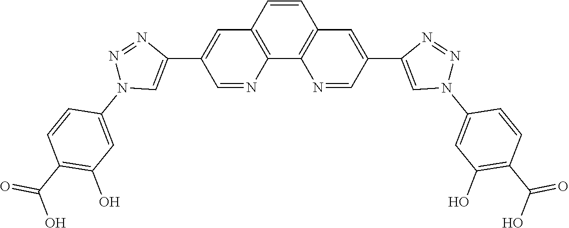 [SMILES: O=C(O)c1ccc(-n2cc(-c3cnc4c(ccc5cc(-c6cn(-c7ccc(C(=O)O)c(O)c7)nn6)cnc54)c3)nn2)cc1O] |
| 60 | 4,4′-((4-(cyclopentylcarbamoyl)pyridine-2,6- diyl)bis(1H-1,2,3-triazole-4,1-diyl))bis(2- hydroxybenzoic acid) | 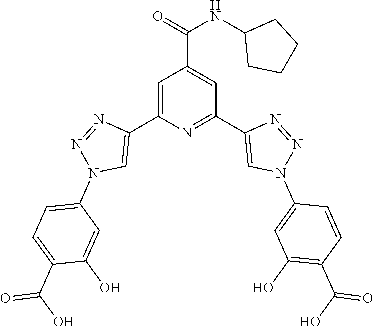 |
| 61 | 4,4′-((4-(dipropylcarbamoyl)pyridine-2,6- diyl)bis(1H-1,2,3-triazole-4,1-diyl))bis(2- hydroxybenzoic acid) | 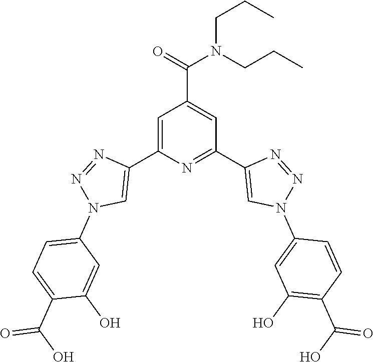 |
| 62 | 4,4′-((4-(di-sec-butylcarbamoyl)pyridine-2,6- diyl)bis(1H-1,2,3-triazole-4,1-diyl))bis(2- hydroxybenzoic acid) | 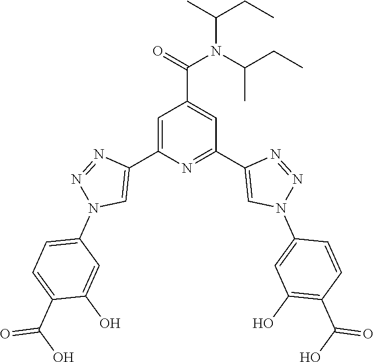 |
| 63 | 4,4′-(naphthalene-2,7-diylbis(1H-1,2,3- triazole-4,1-diyl))bis(2-hydroxybenzoic acid) | 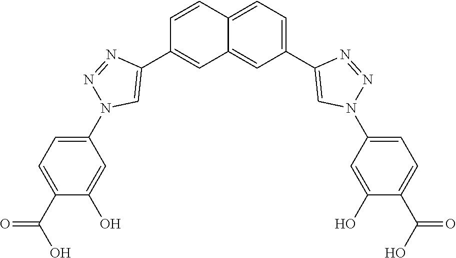 |
| 64 | 4,4′-(naphthalene-2,3-diylbis(1H-1,2,3- triazole-4,1-diyl))bis(2-hydroxybenzoic acid) | 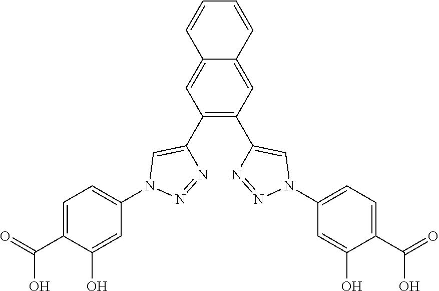 [SMILES: O=C(O)c1ccc(-n2cc(-c3cc4ccccc4cc3-c3cn(-c4ccc(C(=O)O)c(O)c4)nn3)nn2)cc1O] |
| 65 | 4,4′-((4-(dibutylcarbamoyl)pyridine-2,6- diyl)bis(1H-1,2,3-triazole-4,1-diyl))bis(2- hydroxybenzoic acid) | 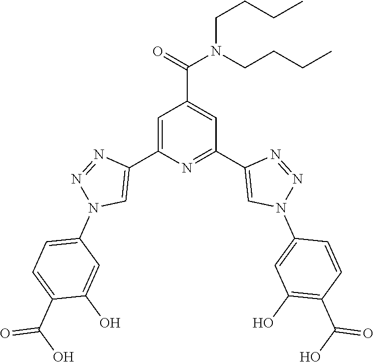 |
| 66 | 4,4′-((4-((2- hydroxyethyl)carbamoyl)pyridine-2,6- diyl)bis(1H-1,2,3-triazole-4,1-diyl))bis(2- hydroxybenzoic acid) | 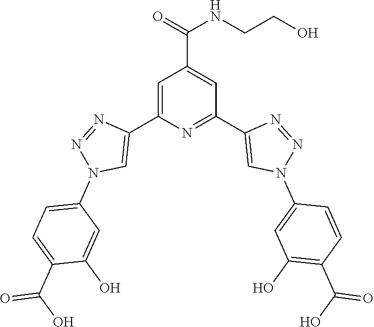 |
| 67 | 4,4′-((4-(cyclohexylcarbamoyl)pyridine-2,6- diyl)bis(1H-1,2,3-triazole-4,1-diyl))bis(2- hydroxybenzoic acid) | 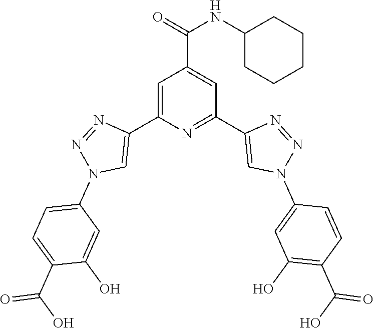 |
| 68 | 4,4′-((4-(benzylcarbamoyl)pyridine-2,6- diyl)bis(1H-1,2,3-triazole-4,1-diyl))bis(2- hydroxybenzoic acid) | 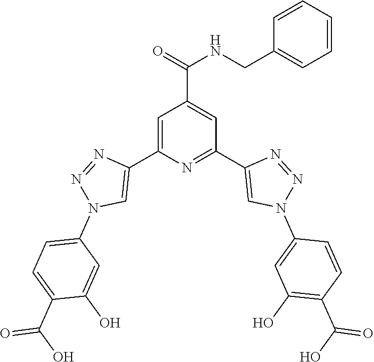 |
| 69 | 4,4′-((4-(4-methylpiperazine-1- carbonyl)pyridine-2,6-diyl)bis(1H-1,2,3- triazole-4,1-diyl))bis(2-hydroxybenzoic acid) | 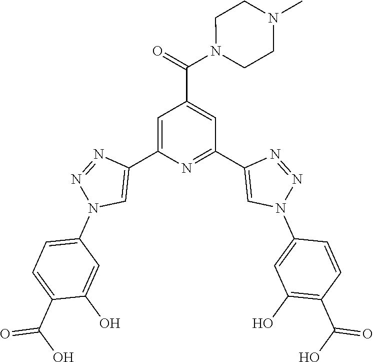 [SMILES: CN1CCN(C(=O)c2cc(-c3cn(-c4ccc(C(=O)O)c(O)c4)nn3)nc(-c3cn(-c4ccc(C(=O)O)c(O)c4)nn3)c2)CC1] |
| 70 | 4-(4-(3-(1-(4-methoxyphenyl)-1H-1,2,3- triazol-4-yl)phenyl)-1H-1,2,3-triazol-1- yl)benzoic acid | 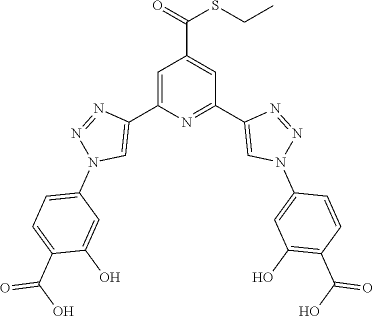 [SMILES: CCSC(=O)c1cc(-c2cn(-c3ccc(C(=O)O)c(O)c3)nn2)nc(-c2cn(-c3ccc(C(=O)O)c(O)c3)nn2)c1] |
| 71 | 4,4′,4″,4″-((((butane-1,4- diylbis(azanediyl))bis(carbonyl))bis(pyridine- 4,2,6-triyl))tetrakis(1H-1,2,3-triazole-4,1- diyl))tetrakis(2-hydroxybenzoic acid) | 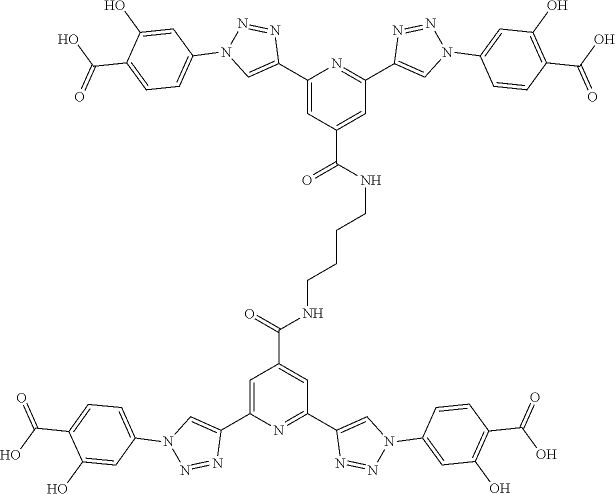 [SMILES: O=C(NCCCCNC(=O)c1cc(-c2cn(-c3ccc(C(=O)O)c(O)c3)nn2)nc(-c2cn(-c3ccc(C(=O)O)c(O)c3)nn2)c1)c1cc(-c2cn(-c3ccc(C(=O)O)c(O)c3)nn2)nc(-c2cn(-c3ccc(C(=O)O)c(O)c3)nn2)c1] |
| 72 | 4,4′-((4-(ethylcarbamoyl)pyridine-2,6- diyl)bis(1H-1,2,3-triazole-4,1-diyl))bis(3,5,6- trichloropicolinic acid) | 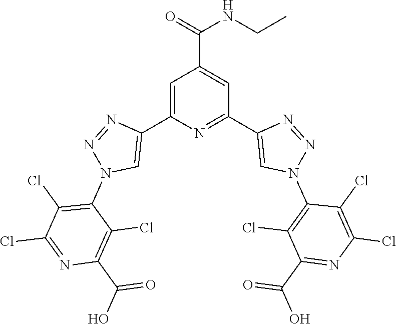 [SMILES: CCNC(=O)c1cc(-c2cn(-c3c(Cl)c(Cl)nc(C(=O)O)c3Cl)nn2)nc(-c2cn(-c3c(Cl)c(Cl)nc(C(=O)O)c3Cl)nn2)c1] |
| 73 | 4,4′-((4-(ethylcarbamoyl)pyridine-2,6- diyl)bis(1H-1,2,3-triazole-4,1-diyl))bis(2- (trifluoromethyl)benzoic acid) | 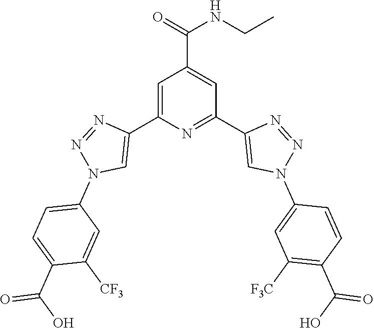 [SMILES: CCNC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 74 | 7,7′-((4-(ethylcarbamoyl)pyridine-2,6- diyl)bis(1H-1,2,3-triazole-4,1-diyl))bis(2- hydroxy-1,8-naphthyridine-4-carboxylic acid) | 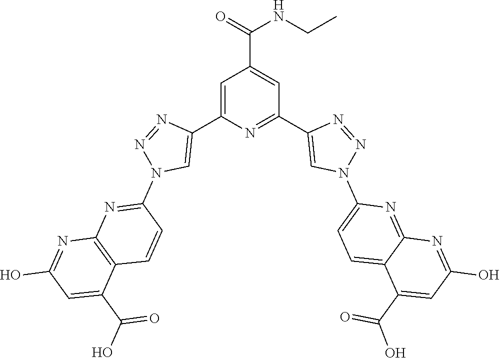 [SMILES: CCNC(=O)c1cc(-c2cn(-c3ccc4c(C(=O)O)cc(O)nc4n3)nn2)nc(-c2cn(-c3ccc4c(C(=O)O)cc(O)nc4n3)nn2)c1] |
| 75 | 5,5′-((4-(ethylcarbamoyl)pyridine-2,6- diyl)bis(1H-1,2,3-triazole-4,1-diyl))bis(2- (trifluoromethyl)benzoic acid) | 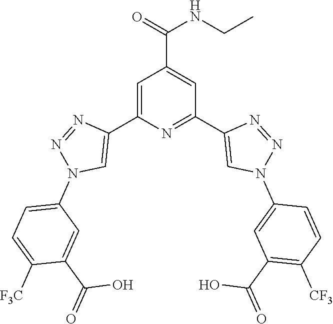 [SMILES: CCNC(=O)c1cc(-c2cn(-c3ccc(C(F)(F)F)c(C(=O)O)c3)nn2)nc(-c2cn(-c3ccc(C(F)(F)F)c(C(=O)O)c3)nn2)c1] |
| 76 | 4,4′-((4-(ethylcarbamoyl)pyridine-2,6- diyl)bis(1H-1,2,3-triazole-4,1-diyl))bis(2- fluorobenzoic acid) | 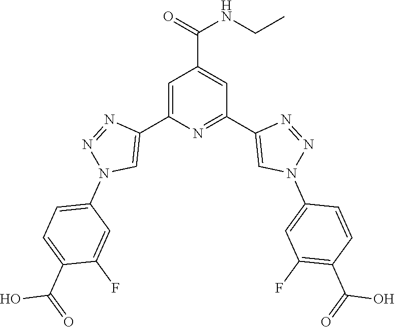 [SMILES: CCNC(=O)c1cc(-c2cn(-c3ccc(C(=O)O)c(F)c3)nn2)nc(-c2cn(-c3ccc(C(=O)O)c(F)c3)nn2)c1] |
| 77 | 5,5′-((4-(ethylcarbamoyl)pyridine-2,6- diyl)bis(1H-1,2,3-triazole-4,1-diyl))bis(3- fluorobenzoic acid) | 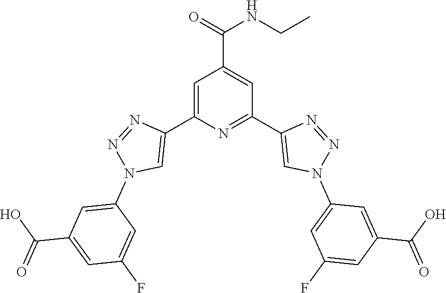 |
| 78 | 2-(1-(1H-benzo[d]imidazol-4-yl)-1H-1,2,3- triazol-4-yl)-6-(1-(1H-benzo[d]imidazol-7- yl)-1H-1,2,3-triazol-4-yl)-N- ethylisonicotinamide | 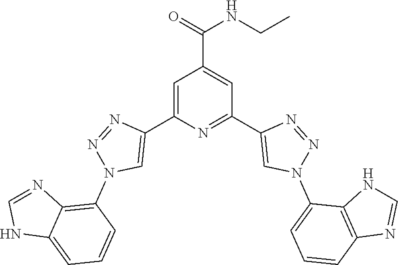 |
| 79 | 4,4′-((4-(methylcarbamoyl)pyridine-2,6- diyl)bis(1H-1,2,3-triazole-4,1-diyl))bis(2- (trifluoromethyl)benzoic acid) | 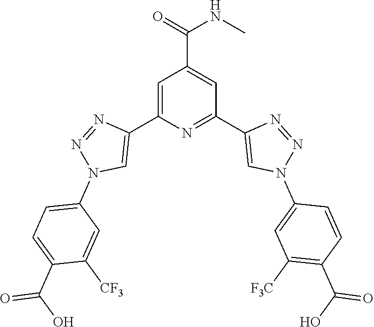 |
| 80 | 4,4′-((4-(morpholine-4-carbonyl)pyridine- 2,6-diyl)bis(1H-1,2,3-triazole-4,1-diyl))bis(2- (trifluoromethyl)benzoic acid) | 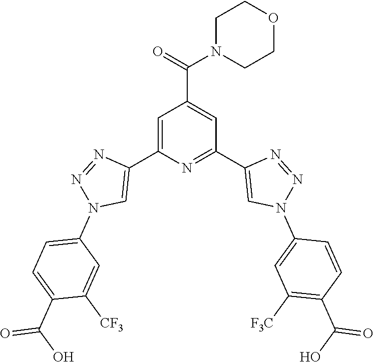 [SMILES: O=C(O)c1ccc(-n2cc(-c3cc(C(=O)N4CCOCC4)cc(-c4cn(-c5ccc(C(=O)O)c(C(F)(F)F)c5)nn4)n3)nn2)cc1C(F)(F)F] |
| 81 | 4,4′-((4-(diethylcarbamoyl)pyridine-2,6- diyl)bis(1H-1,2,3-triazole-4,1-diyl))bis(2- (trifluoromethyl)benzoic acid) | 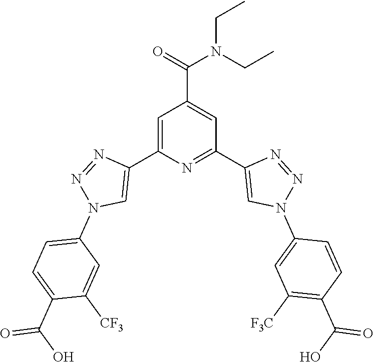 [SMILES: CCN(CC)C(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 82 | 4,4′-((4-carbamoylpyridine-2,6-diyl)bis(1H- 1,2,3-triazole-4,1-diyl))bis(2- (trifluoromethyl)benzoic acid) | 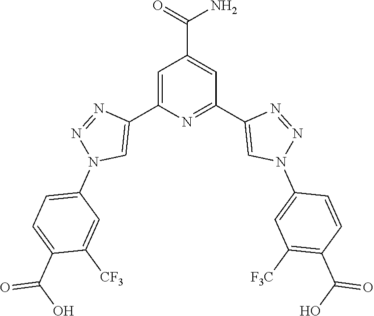 [SMILES: NC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 83 | 4,4′-((4-(ethoxycarbonyl)pyridine-2,6- diyl)bis(1H-1,2,3-triazole-4,1-diyl))bis(2- (trifluoromethyl)benzoic acid) | 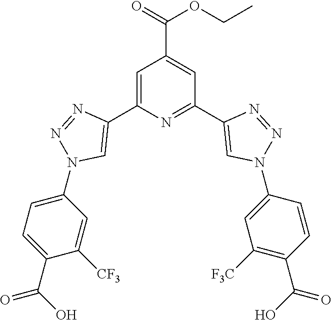 [SMILES: CCOC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 84 | 4,4′-((4-(azetidine-1-carbonyl)pyridine-2,6- diyl)bis(1H-1,2,3-triazole-4,1-diyl))bis(2- hydroxybenzoic acid) | 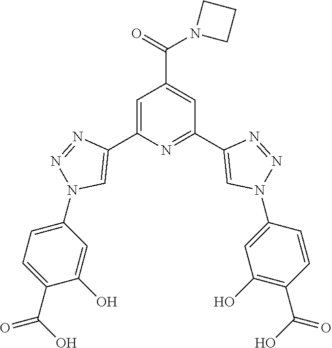 [SMILES: O=C(O)c1ccc(-n2cc(-c3cc(C(=O)N4CCC4)cc(-c4cn(-c5ccc(C(=O)O)c(O)c5)nn4)n3)nn2)cc1O] |
| 85 | 4,4′-((4-(ethyl(methyl)carbamoyl)pyridine- 2,6-diyl)bis(1H-1,2,3-triazole-4,1-diyl))bis(2- hydroxybenzoic acid) | 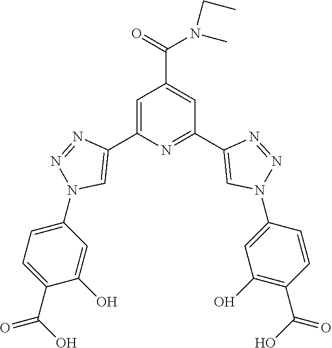 [SMILES: CCN(C)C(=O)c1cc(-c2cn(-c3ccc(C(=O)O)c(O)c3)nn2)nc(-c2cn(-c3ccc(C(=O)O)c(O)c3)nn2)c1] |
| 86 | N-ethyl-2,6-bis(1-(4-(2,2,2- trifluoroacetyl)phenyl)-1H-1,2,3-triazol-4- yl)isonicotinamide | 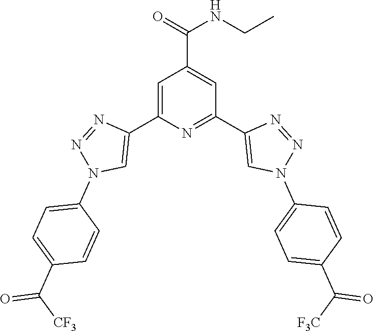 [SMILES: CCNC(=O)c1cc(-c2cn(-c3ccc(C(=O)C(F)(F)F)cc3)nn2)nc(-c2cn(-c3ccc(C(=O)C(F)(F)F)cc3)nn2)c1] |
| 87 | 4,4′-(pyridine-2,6-diylbis(1H-1,2,3-triazole- 4,1-diyl))bis(2-(trifluoromethyl)benzoic acid) | 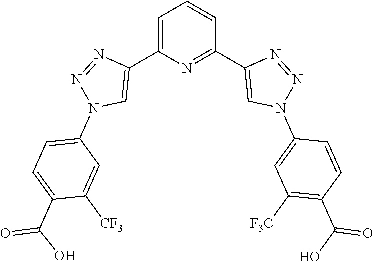 |
| 88 | 4,4′-((4-(cyclopropylcarbamoyl)pyridine-2,6- diyl)bis(1H-1,2,3-triazole-4,1-diyl))bis(2- (trifluoromethyl)benzoic acid) | 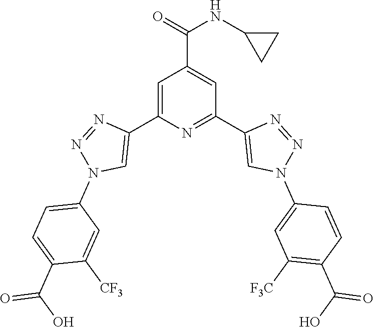 |
| 89 | 4,4′-((4-(butylcarbamoyl)pyridine-2,6- diyl)bis(1H-1,2,3-triazole-4,1-diyl))bis(2- (trifluoromethyl)benzoic acid) | 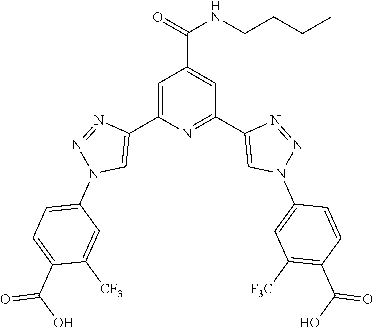 |
| 90 | 5,5′-((4-(diethylcarbamoyl)pyridine-2,6- diyl)bis(1H-1,2,3-triazole-4,1-diyl))bis(2- (trifluoromethyl)benzoic acid) | 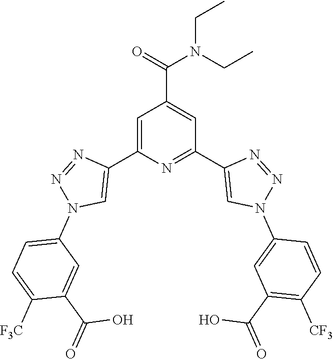 |
| 91 | 5,5′-((4-(morpholine-4-carbonyl)pyridine- 2,6-diyl)bis(1H-1,2,3-triazole-4,1-diyl))bis(2- (trifluoromethyl)benzoic acid) | 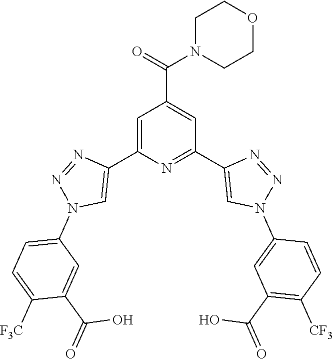 |
| 92 | 4,4′-(pyridazine-3,6-diylbis(1H-1,2,3- triazole-4,1-diyl))bis(2-hydroxybenzoic acid) | 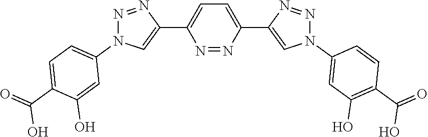 [SMILES: O=C(O)c1ccc(-n2cc(-c3ccc(-c4cn(-c5ccc(C(=O)O)c(O)c5)nn4)nn3)nn2)cc1O] |
| 93 | 5,5′-((4-(ethylcarbamoyl)pyridine-2,6- diyl)bis(1H-1,2,3-triazole-4,1-diyl))bis(3- (trifluoromethyl)benzoic acid) | 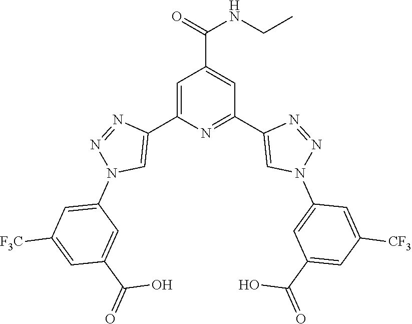 [SMILES: CCNC(=O)c1cc(-c2cn(-c3cc(C(=O)O)cc(C(F)(F)F)c3)nn2)nc(-c2cn(-c3cc(C(=O)O)cc(C(F)(F)F)c3)nn2)c1] |
| 94 | 4,4′-((4-carboxypyridine-2,6-diyl)bis(1H- 1,2,3-triazole-4,1-diyl))bis(2- (trifluoromethyl)benzoic acid) | 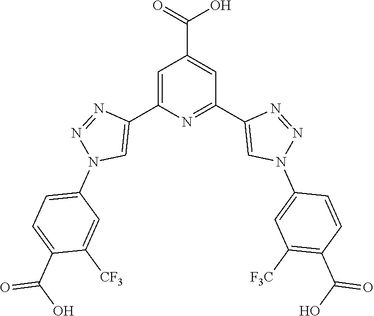 [SMILES: O=C(O)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 95 | 3,3′-(((4-(ethylcarbamoyl)pyridine-2,6- diyl)bis(1H-1,2,3-triazole-4,1-diyl))bis(4,1- phenylene))dipropionic acid | 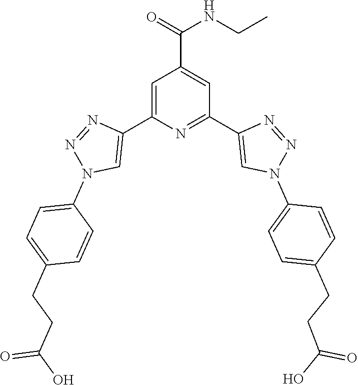 [SMILES: CCNC(=O)c1cc(-c2cn(-c3ccc(CCC(=O)O)cc3)nn2)nc(-c2cn(-c3ccc(CCC(=O)O)cc3)nn2)c1] |
| 96 | 4,4′-(((4-(ethylcarbamoyl)pyridine-2,6- diyl)bis(1H-1,2,3-triazole-4,1-diyl))bis(4,1- phenylene))dibutyric acid | 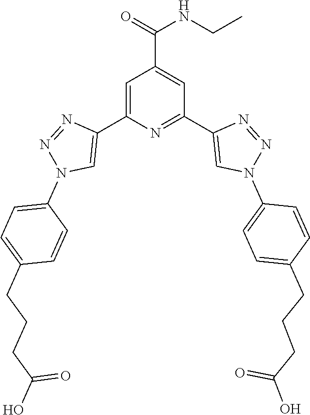 [SMILES: CCNC(=O)c1cc(-c2cn(-c3ccc(CCCC(=O)O)cc3)nn2)nc(-c2cn(-c3ccc(CCCC(=O)O)cc3)nn2)c1] |
| 97 | 4,4′-((4-(ethylcarbamoyl)pyridine-2,6- diyl)bis(1H-1,2,3-triazole-4,1- diyl))diphthalic acid | 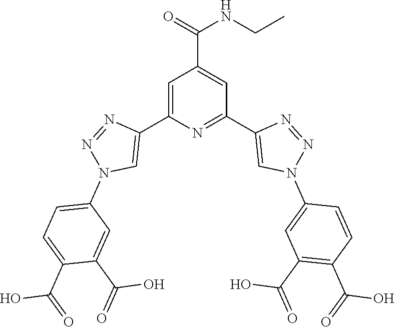 |
| 98 | 4,4′-((4-(ethylcarbamoyl)pyridine-2,6- diyl)bis(1H-1,2,3-triazole-4,1-diyl))bis(2- methoxybenzoic acid) | 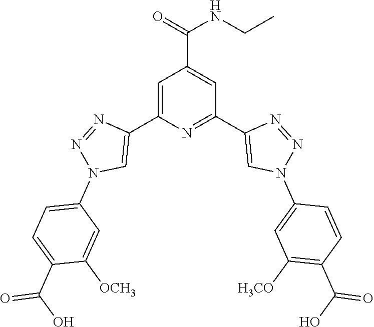 |
| 99 | 5,5′-((4-(ethylcarbamoyl)pyridine-2,6- diyl)bis(1H-1,2,3-triazole-4,1- diyl))diisophthalic acid | 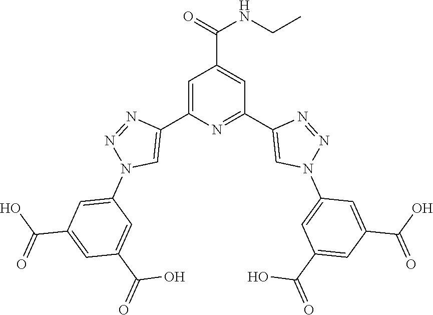 |
| 100 | 4,4′-((4-(ethylcarbamoyl)pyridine-2,6- diyl)bis(1H-1,2,3-triazole-4,1-diyl))bis(3- hydroxybenzoic acid) | 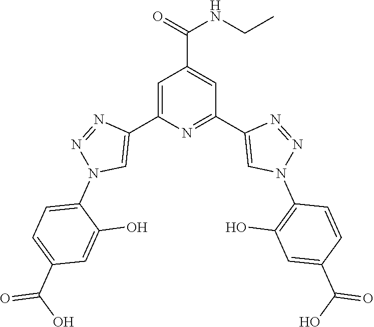 |
| 101 | diethyl (3-(4-(6-(1-(3- (diethoxyphosphoryl)propyl)-1H-1,2,3- triazol-4-yl)-4-(ethylcarbamoyl) 198yridine- 2-yl)-1H-1,2,3-triazol-1-yl)propyl) phosphonate | 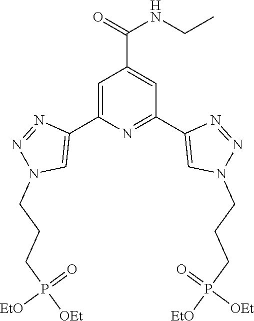 |
| 102 | 4,4′-((4-(ethylcarbamoyl)pyridine-2,6- diyl)bis(1H-1,2,3-triazole-4,1-diyl))bis(2- methylbenzoic acid) | 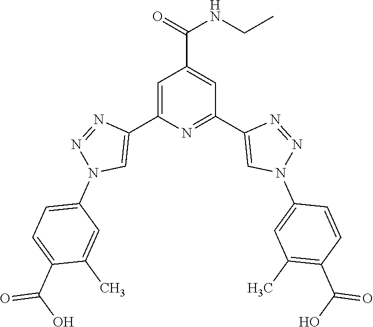 [SMILES: CCNC(=O)c1cc(-c2cn(-c3ccc(C(=O)O)c(C)c3)nn2)nc(-c2cn(-c3ccc(C(=O)O)c(C)c3)nn2)c1] |
| 103 | 4,4′-((5-carboxy-1,3-phenylene)bis(1H-1,2,3- triazole-4,1-diyl))bis(2- (trifluoromethyl)benzoic acid) | 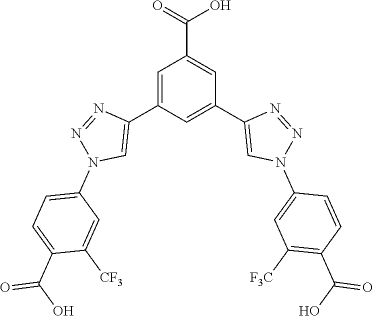 [SMILES: O=C(O)c1cc(-c2cn(-c3ccc(C(=O)O)c(C(F)(F)F)c3)nn2)cc(-c2cn(-c3ccc(C(=O)O)c(C(F)(F)F)c3)nn2)c1] |
| 104 | 2,2′-((4-(ethylcarbamoyl)pyridine-2,6- diyl)bis(1H-1,2,3-triazole-4,1-diyl))bis(3- (trifluoromethyl)benzoic acid) | 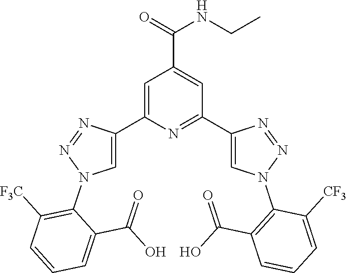 [SMILES: CCNC(=O)c1cc(-c2cn(-c3c(C(=O)O)cccc3C(F)(F)F)nn2)nc(-c2cn(-c3c(C(=O)O)cccc3C(F)(F)F)nn2)c1] |
| 105 | 4,4′-((4-((2- hydroxyethyl)carbamoyl)pyridine-2,6- diyl)bis(1H-1,2,3-triazole-4,1-diyl))bis(2- (trifluoromethyl)benzoic acid) | 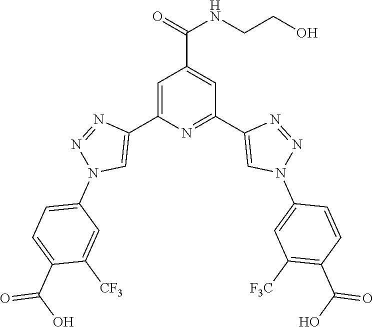 |
| 106 | 4,4′-((4-(ethylcarbamoyl)pyridine-2,6- diyl)bis(1H-1,2,3-triazole-4,1-diyl))bis(2- nitrobenzoic acid) | 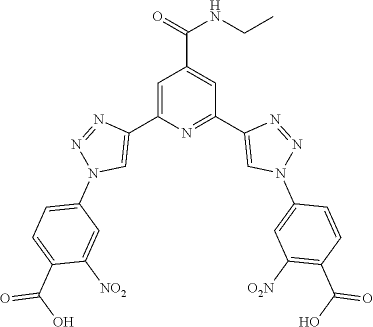 |
| 107 | 4,4′-((4-((3,3,3- trifluoropropyl)carbamoyl)pyridine-2,6- diyl)bis(1H-1,2,3-triazole-4,1-diyl))bis(2- (trifluoromethyl)benzoic acid) | 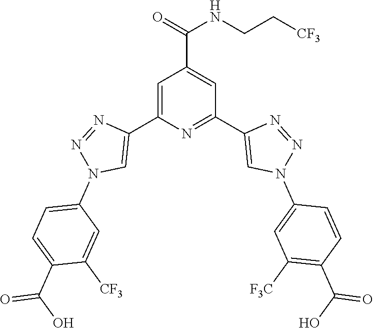 [SMILES: O=C(NCCC(F)(F)F)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 108 | 4,4′,4″,4″-((((butane-1,4- diylbis(azanediyl))bis(carbonyl))bis(pyridine- 4,2,6-triyl))tetrakis(1H-1,2,3-triazole-4,1- diyl))tetrakis(2-(trifluoromethyl)benzoic acid) | 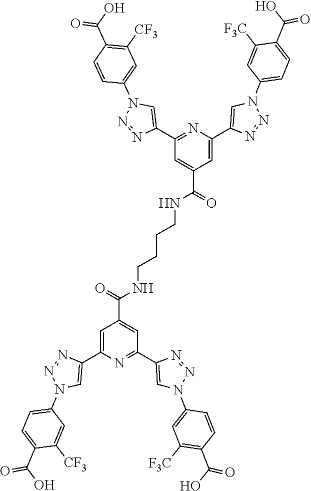 [SMILES: O=C(NCCCCNC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 109 | (4-(4-(4-(ethylcarbamoyl)-6-(1-(4- phosphonophenyl)-1H-1,2,3-triazol-4- yl)200yridine-2-yl)-1H-1,2,3-triazol-1- yl)phenyl)phosphonic acid | 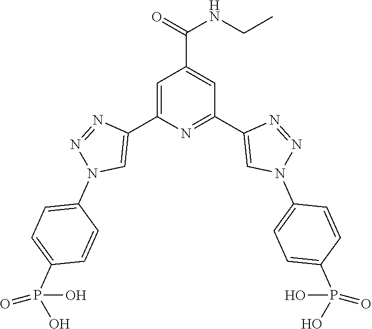 |
| 110 | 2,2′-((4,4′-((4-(ethylcarbamoyl)pyridine-2,6- diyl)bis(1H-1,2,3-triazole-4,1-diyl))bis(2- (trifluoromethyl)benzoyl))bis(azanediyl)) diacetic acid | 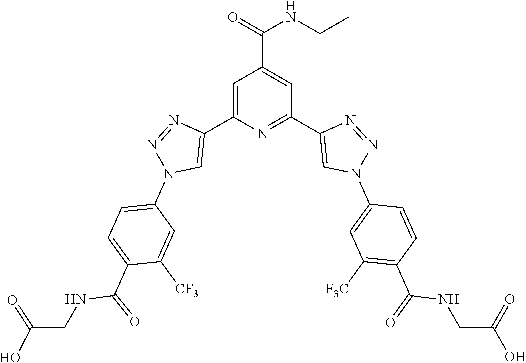 [SMILES: CCNC(=O)c1cc(-c2cn(-c3ccc(C(=O)NCC(=O)O)c(C(F)(F)F)c3)nn2)nc(-c2cn(-c3ccc(C(=O)NCC(=O)O)c(C(F)(F)F)c3)nn2)c1] |
| 111 | dimethyl 2,2′-((4,4′-((4- (ethylcarbamoyl)pyridine-2,6-diyl)bis(1H- 1,2,3-triazole-4,1-diyl))bis(2- (trifluoromethyl)benzoyl))bis(azanediyl)) diacetate | 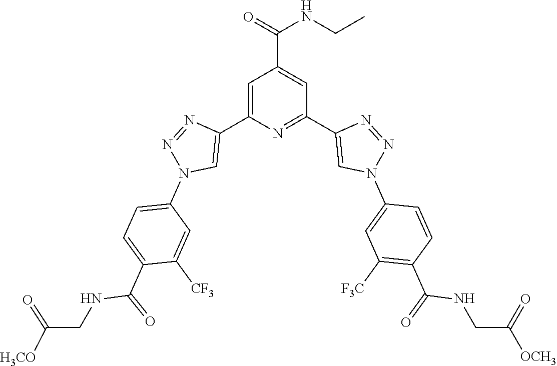 [SMILES: CCNC(=O)c1cc(-c2cn(-c3ccc(C(=O)NCC(=O)OC)c(C(F)(F)F)c3)nn2)nc(-c2cn(-c3ccc(C(=O)NCC(=O)OC)c(C(F)(F)F)c3)nn2)c1] |
| 112 | (2S,2′S)-2,2′-((4,4′-((4- (ethylcarbamoyl)pyridine-2,6-diyl)bis(1H- 1,2,3-triazole-4,1-diyl))bis(2- (trifluoromethyl)benzoyl))bis(azanediyl)) disuccinic acid | 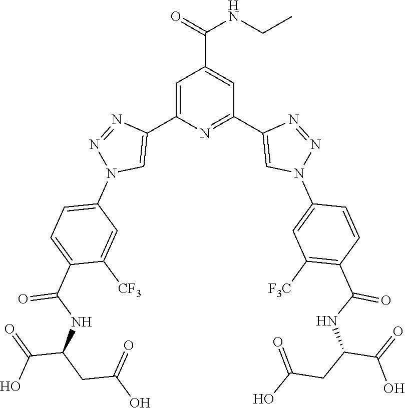 |
| 113 | 2,2′-((2,2′-((4,4′-((4- (ethylcarbamoyl)pyridine-2,6-diyl)bis(1H- 1,2,3-triazole-4,1-diyl))bis(2- (trifluoromethyl)benzoyl))bis(azanediyl))bis (acetyl))bis(azanediyl))diacetic acid | 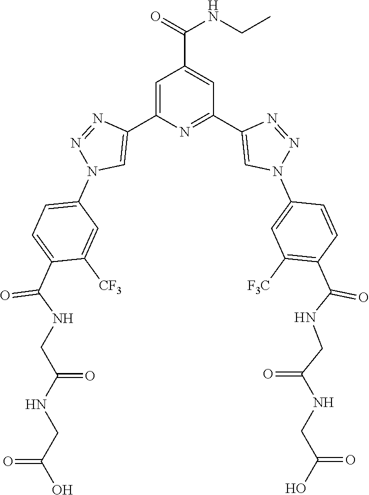 |
| 114 | 2,6-bis(1-(4-cyano-3- (trifluoromethyl)phenyl)-1H-1,2,3-triazol-4- yl)-N-ethylisonicotinamide | 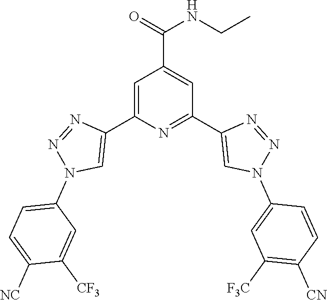 [SMILES: CCNC(=O)c1cc(-c2cn(-c3ccc(C#N)c(C(F)(F)F)c3)nn2)nc(-c2cn(-c3ccc(C#N)c(C(F)(F)F)c3)nn2)c1] |
| 115 | 4,4′-((5-carboxy-1,3-phenylene)bis(1H-1,2,3- triazole-1,4-diyl))bis(2- (trifluoromethyl)benzoic acid) | 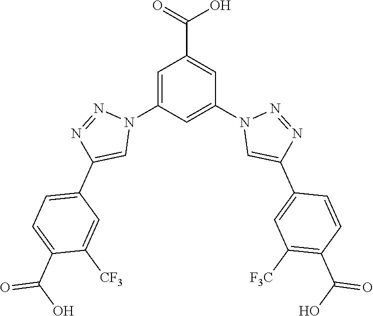 [SMILES: O=C(O)c1cc(-n2cc(-c3ccc(C(=O)O)c(C(F)(F)F)c3)nn2)cc(-n2cc(-c3ccc(C(=O)O)c(C(F)(F)F)c3)nn2)c1] |
| 116 | 4,4′-((5-(ethylcarbamoyl)-1,3- phenylene)bis(1H-1,2,3-triazole-1,4- diyl))bis(2-(trifluoromethyl)benzoic acid) | 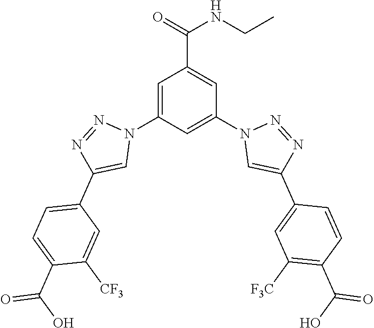 [SMILES: CCNC(=O)c1cc(-n2cc(-c3ccc(C(=O)O)c(C(F)(F)F)c3)nn2)cc(-n2cc(-c3ccc(C(=O)O)c(C(F)(F)F)c3)nn2)c1] |
| 117 | 4,4′-(thiophene-2,5-diylbis(1H-1,2,3-triazole- 4,1-diyl))bis(2-(trifluoromethyl)benzoic acid) | 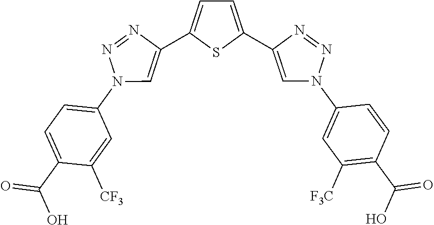 [SMILES: O=C(O)c1ccc(-n2cc(-c3ccc(-c4cn(-c5ccc(C(=O)O)c(C(F)(F)F)c5)nn4)s3)nn2)cc1C(F)(F)F] |
| 118 | 4,4′-(furan-2,5-diylbis(1H-1,2,3-triazole-4,1- diyl))bis(2-(trifluoromethyl)benzoic acid) | 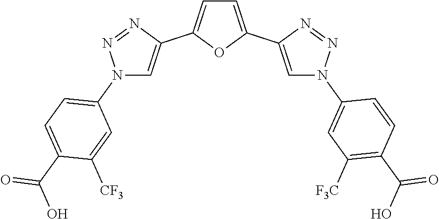 [SMILES: O=C(O)c1ccc(-n2cc(-c3ccc(-c4cn(-c5ccc(C(=O)O)c(C(F)(F)F)c5)nn4)o3)nn2)cc1C(F)(F)F] |
| 119 | 3′-(4-(4-(ethylcarbamoyl)-6-(1-(3′- (trifluoromethyl)-[1,1′-biphenyl]-3-yl)-1H- 1,2,3-triazol-4-yl)pyridin-2-yl)-1H-1,2,3- triazol-1-yl)-3-(trifluoromethyl)-[1,1′- biphenyl]-4-carboxylic acid | 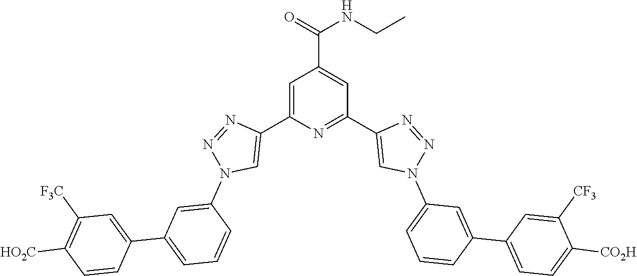 [SMILES: CCNC(=O)c1cc(-c2cn(-c3cccc(-c4ccc(C(=O)O)c(C(F)(F)F)c4)c3)nn2)nc(-c2cn(-c3cccc(-c4ccc(C(=O)O)c(C(F)(F)F)c4)c3)nn2)c1] |
| 120 | 4,4′-((4-(ethylcarbamoyl)pyridine-2,6- diyl)bis(1H-1,2,3-triazole-4,1-diyl))bis(2- cyanobenzoic acid) | 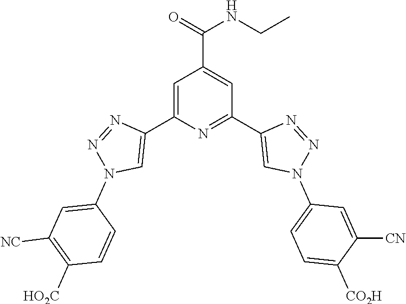 [SMILES: CCNC(=O)c1cc(-c2cn(-c3ccc(C(=O)O)c(C#N)c3)nn2)nc(-c2cn(-c3ccc(C(=O)O)c(C#N)c3)nn2)c1] |
| 121 | 4,4′-((4-(ethylcarbamoyl)pyridine-2,6- diyl)bis(1H-1,2,3-triazole-4,1-diyl))bis(2- chlorobenzoic acid) | 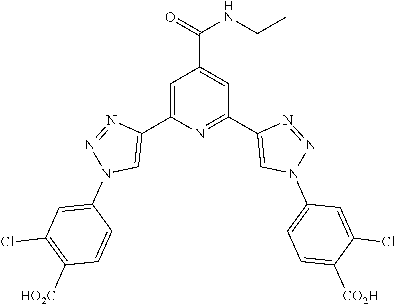 [SMILES: CCNC(=O)c1cc(-c2cn(-c3ccc(C(=O)O)c(Cl)c3)nn2)nc(-c2cn(-c3ccc(C(=O)O)c(Cl)c3)nn2)c1] |
| 122 | p-(4-{p-[1-(p-Aminophenyl)-1H-1,2,3- triazol-4-yl]phenyl}-1H-1,2,3-triazol-1- yl)aniline | 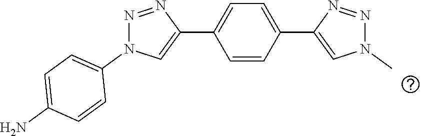 [SMILES: Cn1cc(-c2ccc(-c3cn(-c4ccc(N)cc4)nn3)cc2)nn1] |
| 123 | p-(4-{p-[1-(p-Carboxyphenyl)-1H-1,2,3- triazol-4-yl]phenyl}-1H-1,2,3-triazol-1- yl)benzoic acid | 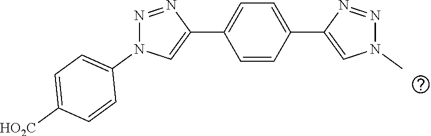 [SMILES: Cn1cc(-c2ccc(-c3cn(-c4ccc(C(=O)O)cc4)nn3)cc2)nn1] |
| 124 | 2-(4-{p-[1-(1,3-Dimethyl-2-imidazolin-3- ium-2-yl)-1H-1,2,3-triazol-4-yl]phenyl}-1H- 1,2,3-triazol-1-yl)-1,3-dimethyl-2- imidazolin-3-ium | 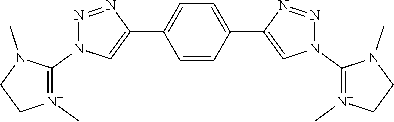 [SMILES: CN1CC[N+](C)=C1n1cc(-c2ccc(-c3cn(C4=[N+](C)CCN4C)nn3)cc2)nn1] |
| 125 | {4-[p-(1-Nicotinoyl-1H-1,2,3-triazol-4- yl)phenyl]-1H-1,2,3-triazol-1-yl} (3- pyridyl)methanone | 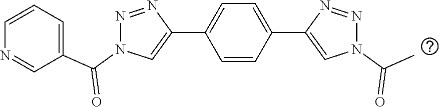 [SMILES: CC(=O)n1cc(-c2ccc(-c3cn(C(=O)c4cccnc4)nn3)cc2)nn1] |
| 126 | 1-(4-Coumarinyl)-4-{p-[1-(4-coumarinyl)- 1H-1,2,3-triazol-4-yl]phenyl}-1H-1,2,3- triazole | 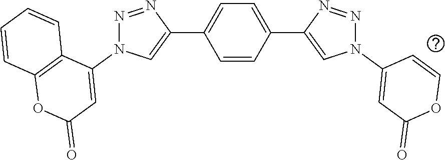 [SMILES: O=c1cc(-n2cc(-c3ccc(-c4cn(-c5cc(=O)oc6ccccc56)nn4)cc3)nn2)cco1] |
| 127 | 1-(1H-1,3-Benzimidazol-2-yl)-4-{p-[1-(1H- 1,3-benzimidazol-2-yl)-1H-1,2,3-triazol-4- yl]phenyl}-1H-1,2,3-triazole | 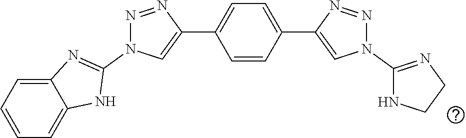 [SMILES: c1ccc2[nH]c(-n3cc(-c4ccc(-c5cn(C6=NCCN6)nn5)cc4)nn3)nc2c1] |
| 128 | 1-[5-(Hydroxymethyl)-4-[4-(p-{ 1-[2- (hydroxymethyl)-5-(5-methyl-2,4-dioxo- 1,2,3,4-tetrahydro-1-pyrimidinyl)tetrahydro- 3-furyl]-1H-1,2,3-triazol-4-yl}phenyl)-1H- 1,2,3-triazol-1-yl]tetrahydro-2-furyl]-5- methyl-2,4(1H,3H)-pyrimidinedione | 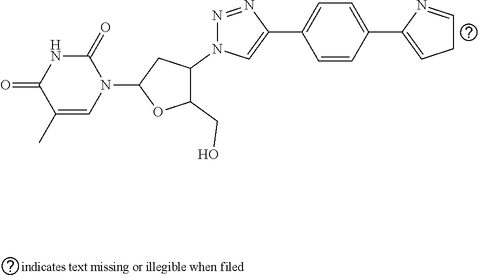 [SMILES: Cc1cn(C2CC(n3cc(-c4ccc(C5=CCC=N5)cc4)nn3)C(CO)O2)c(=O)[nH]c1=O] |
| 129 | 1-(p-Methoxyphenyl)-4-{p-[1-(p- methoxyphenyl)-1H-1,2,3-triazol-4- yl]phenyl}-1H-1,2,3-triazole | 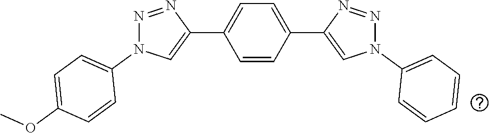 [SMILES: COc1ccc(-n2cc(-c3ccc(-c4cn(-c5ccccc5)nn4)cc3)nn2)cc1] |
| 130 | o-(4-{p-[1-(o-Carboxyphenyl)-1H-1,2,3- triazol-4-yl]phenyl}-1H-1,2,3-triazol-1- yl)benzoic acid | 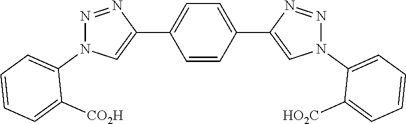 [SMILES: O=C(O)c1ccccc1-n1cc(-c2ccc(-c3cn(-c4ccccc4C(=O)O)nn3)cc2)nn1] |
| 131 | Methyl (4-{p-[1-(methoxycarbonylmethyl)- 1H-1,2,3-triazol-4-yl]phenyl}-1H-1,2,3- triazol-1-yl)acetate | 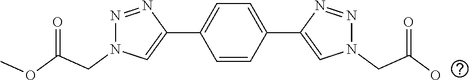 [SMILES: COC(=O)Cn1cc(-c2ccc(-c3cn(CC(=O)O)nn3)cc2)nn1] |
| 132 | 3-(4-{p-[1-(3-Aminopropyl)-1H-1,2,3- triazol-4-yl]phenyl}-1H-1,2,3-triazol-1-yl)-1- propanamine | 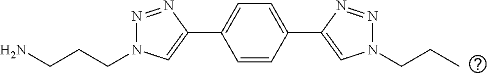 [SMILES: CCCn1cc(-c2ccc(-c3cn(CCCN)nn3)cc2)nn1] |
| 133 | 2-(4-{6-[1-(1,3-Dimethyl-2-imidazolin-3- ium-2-yl)-1H-1,2,3-triazol-4-yl]-3- carbazolyl}-1H-1,2,3-triazol-1-yl)-1,3- dimethyl-2-imidazolin-3-ium | 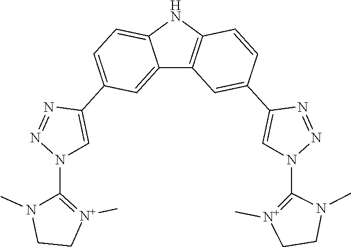 |
| 134 | {4-[6-(1-Nicotinoyl-1H-1,2,3-triazol-4-yl)-3- carbazolyl]-1H-1,2,3-triazol-1-yl}(3- pyridyl)methanone | 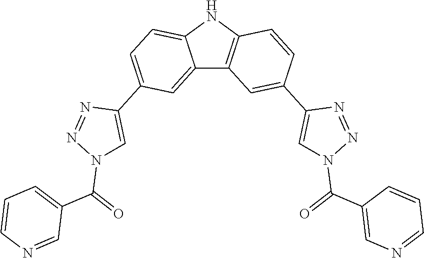 [SMILES: O=C(c1cccnc1)n1cc(-c2ccc3[nH]c4ccc(-c5cn(C(=O)c6cccnc6)nn5)cc4c3c2)nn1] |
| 135 | 1-(4-Coumarinyl)-4-{6-[1-(4-coumarinyl)- 1H-1,2,3-triazol-4-yl]-3-carbazolyl}-1H- 1,2,3-triazole | 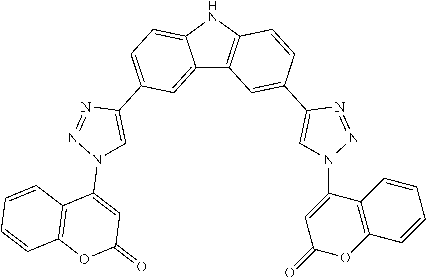 [SMILES: O=c1cc(-n2cc(-c3ccc4[nH]c5ccc(-c6cn(-c7cc(=O)oc8ccccc78)nn6)cc5c4c3)nn2)c2ccccc2o1] |
| 136 | 1-(1H-1,3-Benzimidazol-2-yl)-4-{6-[1-(1H- 1,3-benzimidazol-2-yl)-1H-1,2,3-triazol-4- yl]-3-carbazolyl}-1H-1,2,3-triazole | 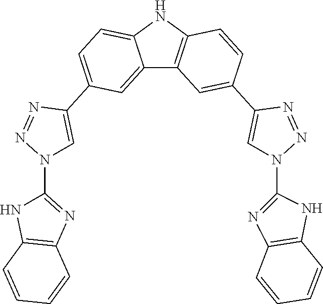 [SMILES: c1ccc2[nH]c(-n3cc(-c4ccc5[nH]c6ccc(-c7cn(-c8nc9ccccc9[nH]8)nn7)cc6c5c4)nn3)nc2c1] |
| 137 | 1-[5-(Hydroxymethyl)-4-[4-(6-{1-[2- (hydroxymethyl)-5-(5-methyl-2,4-dioxo- 1,2,3,4-tetrahydro-1-pyrimidinyl)tetrahydro- 3-furyl]-1H-1,2,3-triazol-4-yl}-3-carbazolyl)- 1H-1,2,3-triazol-1-yl]tetrahydro-2-furyl]-5- methyl-2,4(1H,3H)-pyrimidinedione | 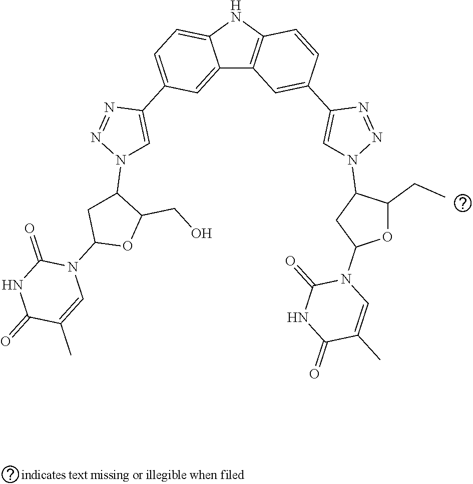 [SMILES: CCC1OC(n2cc(C)c(=O)[nH]c2=O)CC1n1cc(-c2ccc3[nH]c4ccc(-c5cn(C6CC(n7cc(C)c(=O)[nH]c7=O)OC6CO)nn5)cc4c3c2)nn1] |
| 138 | o-(4-{6-[1-(o-Carboxyphenyl)-1H-1,2,3- triazol-4-yl]-3-carbazolyl}-1H-1,2,3-triazol- 1-yl)benzoic acid | 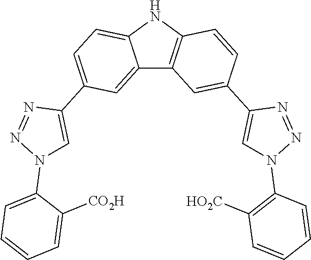 [SMILES: O=C(O)c1ccccc1-n1cc(-c2ccc3[nH]c4ccc(-c5cn(-c6ccccc6C(=O)O)nn5)cc4c3c2)nn1] |
| 139 | 3-(4-{6-[1-(3-Aminopropyl)-1H-1,2,3- triazol-4-yl]-3-carbazolyl}-1H-1,2,3-triazol- 1-yl)-1-propanamine | 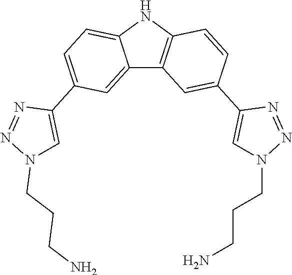 [SMILES: NCCCn1cc(-c2ccc3[nH]c4ccc(-c5cn(CCCN)nn5)cc4c3c2)nn1] |
| 140 | 1,3-Dimethyl-2-(4-{m-[1-(1-methyl-4- methyl-3,4-dihydro-2H-pyrrol-1-ium-5-yl)- 1H-1,2,3-triazol-4-yl]phenyl}-1H-1,2,3- triazol-1-yl)-2-imidazolin-3-ium | 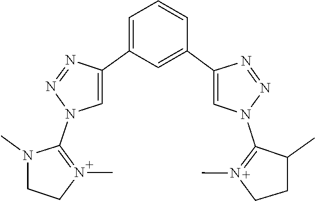 |
| 141 | {4-[m-(1-Nicotinoyl-1H-1,2,3-triazol-4- yl)phenyl]-1H-1,2,3-triazol-1-yl}(3- pyridyl)methanone | 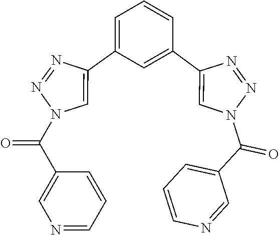 [SMILES: O=C(c1cccnc1)n1cc(-c2cccc(-c3cn(C(=O)c4cccnc4)nn3)c2)nn1] |
| 142 | 1-(4-Coumarinyl)-4-{m-[1-(4-coumarinyl)- 1H-1,2,3-triazol-4-yl]phenyl}-1H-1,2,3- triazole | 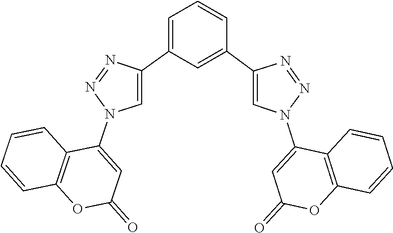 [SMILES: O=c1cc(-n2cc(-c3cccc(-c4cn(-c5cc(=O)oc6ccccc56)nn4)c3)nn2)c2ccccc2o1] |
| 143 | 1-(1H-1,3-Benzimidazol-2-yl)-4-{m-[1-(1H- 1,3-benzimidazol-2-yl)-1H-1,2,3-triazol-4- yl]phenyl}-1H-1,2,3-triazole | 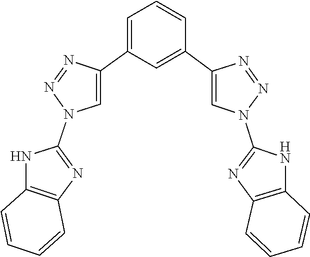 [SMILES: c1cc(-c2cn(-c3nc4ccccc4[nH]3)nn2)cc(-c2cn(-c3nc4ccccc4[nH]3)nn2)c1] |
| 144 | 1-[5-(Hydroxymethyl)-4-[4-(m-{1-[2- (hydroxymethyl)-5-(5-methyl-2,4-dioxo- 1,2,3,4-tetrahydro-1-pyrimidinyl)tetrahydro- 3-furyl]-1H-1,2,3-triazol-4-yl}phenyl)-1H- 1,2,3-triazol-1-yl]tetrahydro-2-furyl]-5- methyl-2,4(1H,3H)-pyrimidinedione | 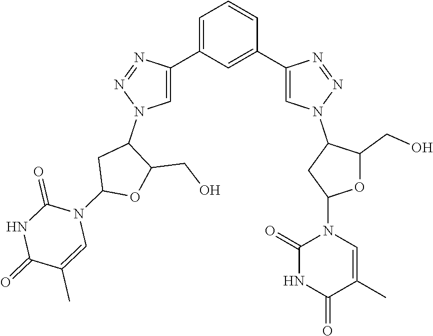 [SMILES: Cc1cn(C2CC(n3cc(-c4cccc(-c5cn(C6CC(n7cc(C)c(=O)[nH]c7=O)OC6CO)nn5)c4)nn3)C(CO)O2)c(=O)[nH]c1=O] |
| 145 | o-(4-{m-[1-(o-Carboxyphenyl)-1H-1,2,3- triazol-4-yl]phenyl}-1H-1,2,3-triazol-1- yl)benzoic acid | 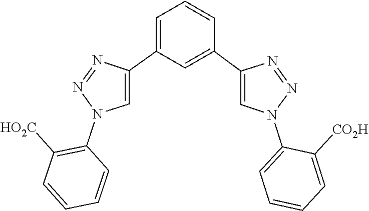 [SMILES: O=C(O)c1ccccc1-n1cc(-c2cccc(-c3cn(-c4ccccc4C(=O)O)nn3)c2)nn1] |
| 146 | Methyl (4-{m-[1-(methoxycarbonylmethyl)- 1H-1,2,3-triazol-4-yl]phenyl}-1H-1,2,3- triazol-1-yl)acetate | 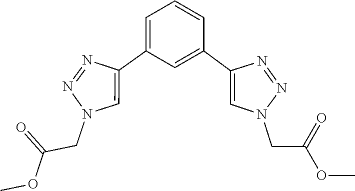 |
| 147 | 3-(4-{m-[1-(3-Aminopropyl)-1H-1,2,3- triazol-4-yl]phenyl}-1H-1,2,3-triazol-1-yl)-1- propanamine | 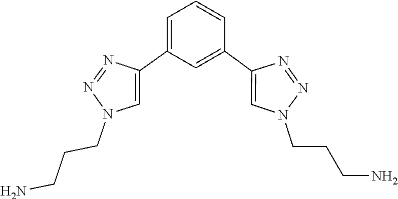 [SMILES: NCCCn1cc(-c2cccc(-c3cn(CCCN)nn3)c2)nn1] |
| 148 | 1,3-Dimethyl-2-(4-{6-[1-(1-methyl-4- methyl-3,4-dihydro-2H-pyrrol-1-ium-5-yl)- 1H-1,2,3-triazol-4-yl]-2-pyridyl}-1H-1,2,3- triazol-1-yl)-2-imidazolin-3-ium | 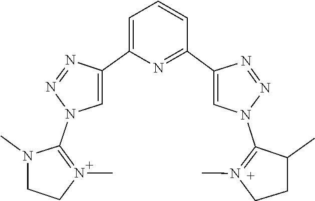 |
| 149 | {4-[6-(1-Nicotinoyl-1H-1,2,3-triazol-4-yl)-2- pyridyl]-1H-1,2,3-triazol-1-yl}(3- pyridyl)methanone | 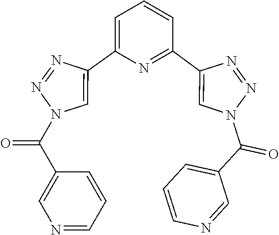 [SMILES: O=C(c1cccnc1)n1cc(-c2cccc(-c3cn(C(=O)c4cccnc4)nn3)n2)nn1] |
| 150 | 1-(4-Coumarinyl)-4-{6-[1-(4-coumarinyl)- 1H-1,2,3-triazol-4-yl]-2-pyridyl}-1H-1,2,3- triazole | 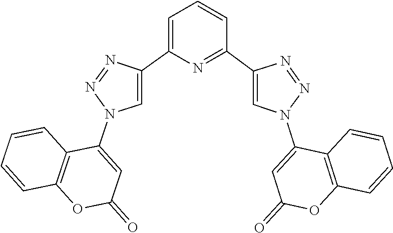 [SMILES: O=c1cc(-n2cc(-c3cccc(-c4cn(-c5cc(=O)oc6ccccc56)nn4)n3)nn2)c2ccccc2o1] |
| 151 | 1-(1H-1,3-Benzimidazol-2-yl)-4-{6-[1-(1H- 1,3-benzimidazol-2-yl)-1H-1,2,3-triazol-4- yl]-2-pyridyl}-1H-1,2,3-triazole | 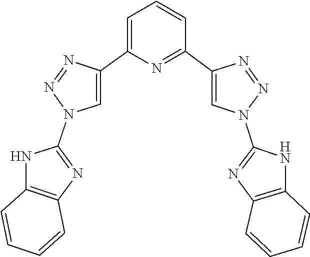 [SMILES: c1cc(-c2cn(-c3nc4ccccc4[nH]3)nn2)nc(-c2cn(-c3nc4ccccc4[nH]3)nn2)c1] |
| 152 | 1-[5-(Hydroxymethyl)-4-[4-(6-{1-[2- (hydroxymethyl)-5-(5-methyl-2,4-dioxo- 1,2,3,4-tetrahydro-1-pyrimidinyl)tetrahydro- 3-furyl]-1H-1,2,3-triazol-4-yl}-2-pyridyl)- 1H-1,2,3-triazol-1-yl]tetrahydro-2-furyl]-5- methyl-2,4(1H,3H)-pyrimidinedione | 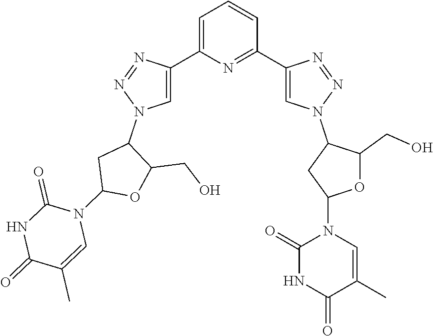 [SMILES: Cc1cn(C2CC(n3cc(-c4cccc(-c5cn(C6CC(n7cc(C)c(=O)[nH]c7=O)OC6CO)nn5)n4)nn3)C(CO)O2)c(=O)[nH]c1=O] |
| 153 | o-(4-{6-[1-(o-Carboxyphenyl)-1H-1,2,3- triazol-4-yl]-2-pyridyl}-1H-1,2,3-triazol-1- yl)benzoic acid | 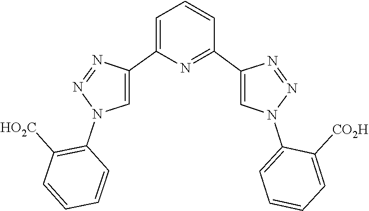 [SMILES: O=C(O)c1ccccc1-n1cc(-c2cccc(-c3cn(-c4ccccc4C(=O)O)nn3)n2)nn1] |
| 154 | Methyl (4-{6-[1-(methoxycarbonylmethyl)- 1H-1,2,3-triazol-4-yl]-2-pyridyl}-1H-1,2,3- triazol-1-yl)acetate | 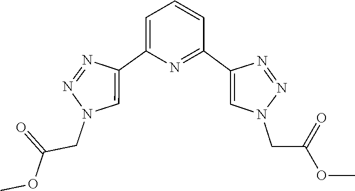 |
| 155 | 3-(4-{6-[1-(3-Aminopropyl)-1H-1,2,3- triazol-4-yl]-2-pyridyl}-1H-1,2,3-triazol-1- yl)-1-propanamine | 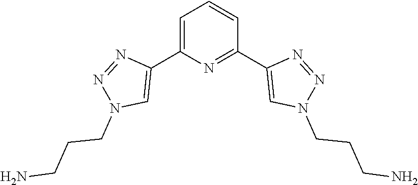 [SMILES: NCCCn1cc(-c2cccc(-c3cn(CCCN)nn3)n2)nn1] |
| 156 | 4-{4-[(3-{[1-(4-Carboxy-3-hydroxyphenyl)- 1H-1,2,3-triazol-4-yl]methyl}ureido)methyl]- 1H-1,2,3-triazol-1-yl}-2-hydroxybenzoic acid | 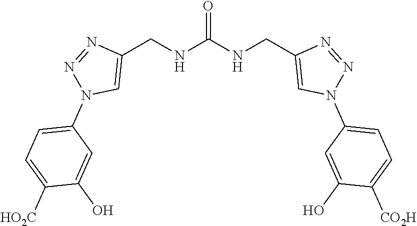 [SMILES: O=C(NCc1cn(-c2ccc(C(=O)O)c(O)c2)nn1)NCc1cn(-c2ccc(C(=O)O)c(O)c2)nn1] |
| 157 | 4-{4-[(3-{[1-(4-Carboxy-3-hydroxyphenyl)- 1H-1,2,3-triazol-4-yl]methyl}ureido)methyl]- 1H-1,2,3-triazol-1-yl}-2-hydroxybenzoic acid | 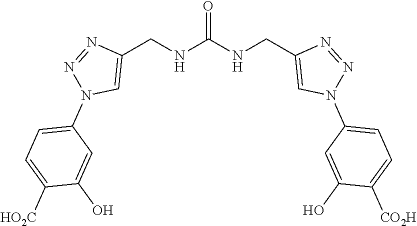 [SMILES: O=C(NCc1cn(-c2ccc(C(=O)O)c(O)c2)nn1)NCc1cn(-c2ccc(C(=O)O)c(O)c2)nn1] |
| 158 | 4-tert-Butoxycarbonyl-2-(4-{6-[1-(3-tert- butoxycarbonyl-1-carboxypropyl)-1H-1,2,3- triazol-4-yl]-3-carbazolyl}-1H-1,2,3-triazol- 1-yl)butyric acid | 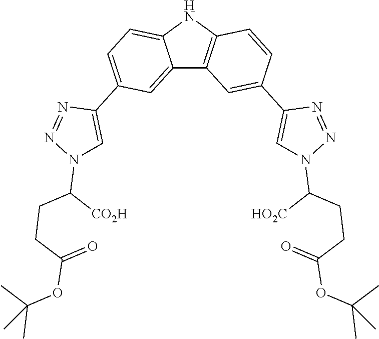 [SMILES: CC(C)(C)OC(=O)CCC(C(=O)O)n1cc(-c2ccc3[nH]c4ccc(-c5cn(C(CCC(=O)OC(C)(C)C)C(=O)O)nn5)cc4c3c2)nn1] |
| 159 | 3-tert-Butoxycarbonyl-2-(4-{6-[1-(2-tert- butoxycarbonyl-1-carboxyethyl)-1H-1,2,3- triazol-4-yl]-3-carbazolyl}-1H-1,2,3-triazol- 1-yl)propionic acid | 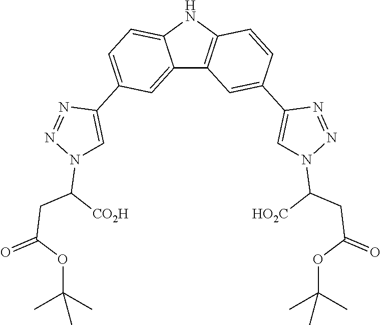 [SMILES: CC(C)(C)OC(=O)CC(C(=O)O)n1cc(-c2ccc3[nH]c4ccc(-c5cn(C(CC(=O)OC(C)(C)C)C(=O)O)nn5)cc4c3c2)nn1] |
| 160 | 2-[tert-Butyl(oxycarbonylamino)]-5-{4-[6- (1-{4-[tert-butyl(oxycarbonylamino)]-4- carboxybutyl}-1H-1,2,3-triazol-4-yl)-3- carbazolyl]-1H-1,2,3-triazol-1-yl} valeric acid | 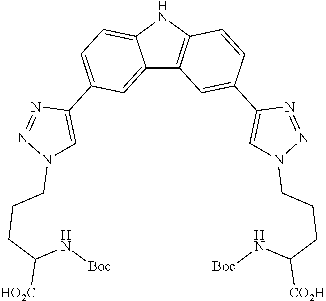 [SMILES: CC(C)(C)OC(=O)NC(CCCn1cc(-c2ccc3[nH]c4ccc(-c5cn(CCCC(NC(=O)OC(C)(C)C)C(=O)O)nn5)cc4c3c2)nn1)C(=O)O] |
| 161 | 4-tert-Butoxycarbonyl-2-(4-{m-[1-(3-tert- butoxycarbonyl-1-carboxypropyl)-1H-1,2,3- triazol-4-yl]phenyl}-1H-1,2,3-triazol-1- yl)butyric acid | 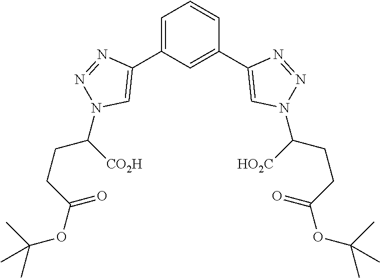 [SMILES: CC(C)(C)OC(=O)CCC(C(=O)O)n1cc(-c2cccc(-c3cn(C(CCC(=O)OC(C)(C)C)C(=O)O)nn3)c2)nn1] |
| 162 | 3-tert-Butoxycarbonyl-2-(4-{m-[1-(2-tert- butoxycarbonyl-1-carboxyethyl)-1H-1,2,3- triazol-4-yl]phenyl}-1H-1,2,3-triazol-1- yl)propionic acid | 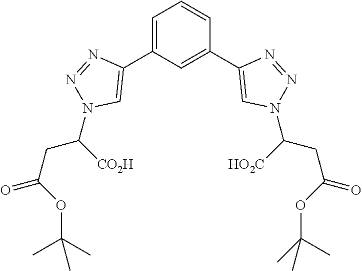 [SMILES: CC(C)(C)OC(=O)CC(C(=O)O)n1cc(-c2cccc(-c3cn(C(CC(=O)OC(C)(C)C)C(=O)O)nn3)c2)nn1] |
| 163 | 2-[tert-Butyl(oxycarbonylamino) ]-5-{4-[m- (1-{4-[tert-butyl(oxycarbonylamino)]-4- carboxybutyl}-1H-1,2,3-triazol-4-yl)phenyl]- 1H-1,2,3-triazol-1-yl} valeric acid | 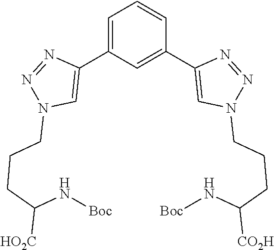 [SMILES: CC(C)(C)OC(=O)NC(CCCn1cc(-c2cccc(-c3cn(CCCC(NC(=O)OC(C)(C)C)C(=O)O)nn3)c2)nn1)C(=O)O] |
| 164 | 4-tert-Butoxycarbonyl-2-(4-{6-[1-(3-tert- butoxycarbonyl-1-carboxypropyl)-1H-1,2,3- triazol-4-yl]-2-pyridyl}-1H-1,2,3-triazol-1- yl)butyric acid | 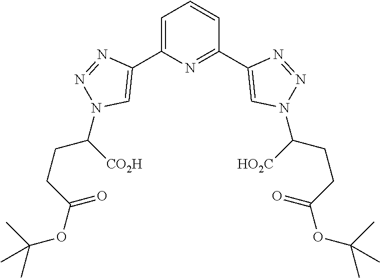 [SMILES: CC(C)(C)OC(=O)CCC(C(=O)O)n1cc(-c2cccc(-c3cn(C(CCC(=O)OC(C)(C)C)C(=O)O)nn3)n2)nn1] |
| 165 | 3-tert-Butoxycarbonyl-2-(4-{6-[1-(2-tert- butoxycarbonyl-1-carboxyethyl)-1H-1,2,3- triazol-4-yl]-2-pyridyl}-1H-1,2,3-triazol-1- yl)propionic acid | 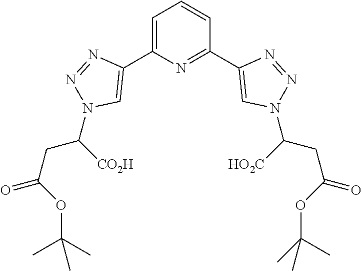 [SMILES: CC(C)(C)OC(=O)CC(C(=O)O)n1cc(-c2cccc(-c3cn(C(CC(=O)OC(C)(C)C)C(=O)O)nn3)n2)nn1] |
| 166 | 2-(4-{6-[1-(1,3-Dicarboxypropyl)-1H-1,2,3- triazol-4-yl]-3-carbazolyl}-1H-1,2,3-triazol- 1-yl)glutaric acid | 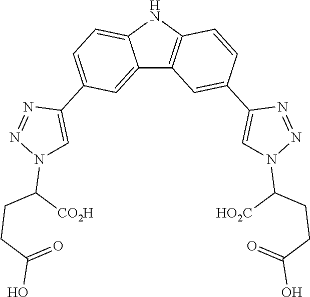 [SMILES: O=C(O)CCC(C(=O)O)n1cc(-c2ccc3[nH]c4ccc(-c5cn(C(CCC(=O)O)C(=O)O)nn5)cc4c3c2)nn1] |
| 167 | 2-(4-{6-[1-(1,2-Dicarboxyethyl)-1H-1,2,3- triazol-4-yl]-3-carbazolyl}-1H-1,2,3-triazol- 1-yl)succinic acid | 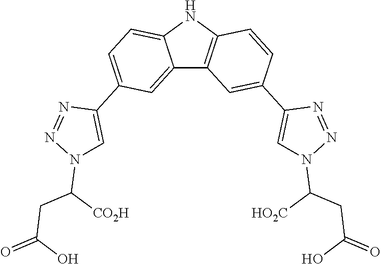 [SMILES: O=C(O)CC(C(=O)O)n1cc(-c2ccc3[nH]c4ccc(-c5cn(C(CC(=O)O)C(=O)O)nn5)cc4c3c2)nn1] |
| 168 | 2-Amino-5-(4-{6-[1-(4-amino-4- carboxybutyl)-1H-1,2,3-triazol-4-yl]-3- carbazolyl}-1H-1,2,3-triazol-1-yl)valeric acid | 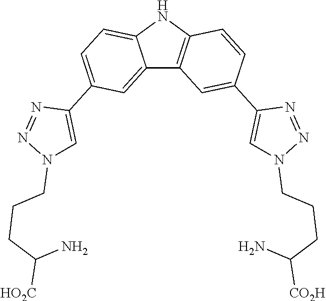 [SMILES: NC(CCCn1cc(-c2ccc3[nH]c4ccc(-c5cn(CCCC(N)C(=O)O)nn5)cc4c3c2)nn1)C(=O)O] |
| 169 | 2-(4-{m-[1-(1,3-Dicarboxypropyl)-1H-1,2,3- triazol-4-yl]phenyl}-1H-1,2,3-triazol-1- yl)glutaric acid | 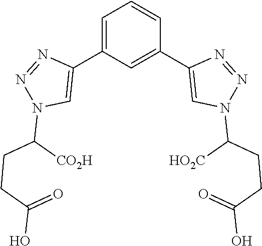 [SMILES: O=C(O)CCC(C(=O)O)n1cc(-c2cccc(-c3cn(C(CCC(=O)O)C(=O)O)nn3)c2)nn1] |
| 170 | 2-(4-{m-[1-(1,2-Dicarboxyethyl)-1H-1,2,3- triazol-4-yl]phenyl}-1H-1,2,3-triazol-1- yl)succinic acid | 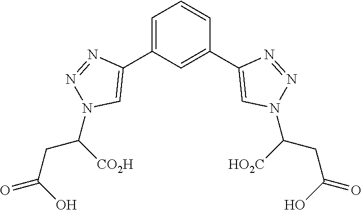 [SMILES: O=C(O)CC(C(=O)O)n1cc(-c2cccc(-c3cn(C(CC(=O)O)C(=O)O)nn3)c2)nn1] |
| 171 | 2-Amino-5-(4-{m-[1-(4-amino-4- carboxybutyl)-1H-1,2,3-triazol-4-yl]phenyl}- 1H-1,2,3-triazol-1-yl)valeric acid | 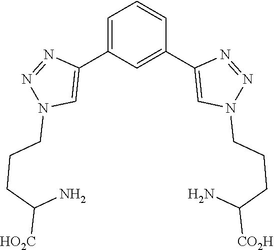 [SMILES: NC(CCCn1cc(-c2cccc(-c3cn(CCCC(N)C(=O)O)nn3)c2)nn1)C(=O)O] |
| 172 | 2-(4-{6-[1-(1,3-Dicarboxypropyl)-1H-1,2,3- triazol-4-yl]-2-pyridyl}-1H-1,2,3-triazol-1- yl)glutaric acid | 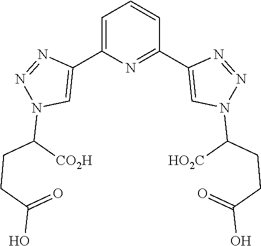 [SMILES: O=C(O)CCC(C(=O)O)n1cc(-c2cccc(-c3cn(C(CCC(=O)O)C(=O)O)nn3)n2)nn1] |
| 173 | 2-(4-{6-[1-(1,2-Dicarboxyethyl)-1H-1,2,3- triazol-4-yl]-2-pyridyl}-1H-1,2,3-triazol-1- yl)succinic acid | 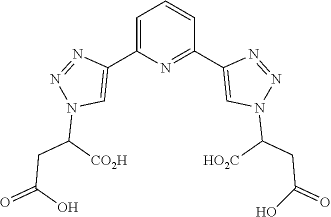 [SMILES: O=C(O)CC(C(=O)O)n1cc(-c2cccc(-c3cn(C(CC(=O)O)C(=O)O)nn3)n2)nn1] |
| 174 | 2-Amino-5-(4-{6-[1-(4-amino-4- carboxybutyl)-1H-1,2,3-triazol-4-yl]-2- pyridyl}-1H-1,2,3-triazol-1-yl)valeric acid | 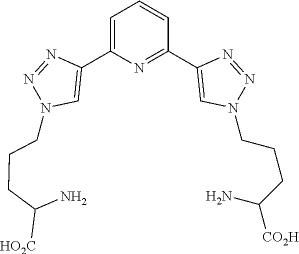 [SMILES: NC(CCCn1cc(-c2cccc(-c3cn(CCCC(N)C(=O)O)nn3)n2)nn1)C(=O)O] |
| 175 | p-(4-{m-[1-(p-Aminophenyl)-1H-1,2,3- triazol-4-yl]phenyl}-1H-1,2,3-triazol-1- yl)benzoic acid | 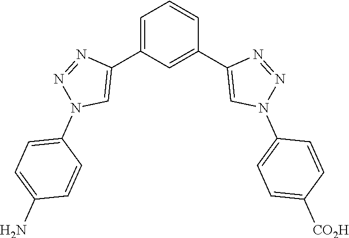 [SMILES: Nc1ccc(-n2cc(-c3cccc(-c4cn(-c5ccc(C(=O)O)cc5)nn4)c3)nn2)cc1] |
| 176 | 4-(4-{m-[1-(p-Carboxyphenyl)-1H-1,2,3- triazol-4-yl]phenyl}-1H-1,2,3-triazol-1-yl)-2- hydroxybenzoic acid | 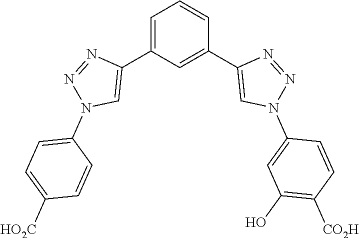 [SMILES: O=C(O)c1ccc(-n2cc(-c3cccc(-c4cn(-c5ccc(C(=O)O)c(O)c5)nn4)c3)nn2)cc1] |
| 177 | p-(4-{m-[1-(Methoxycarbonylmethyl)-1H- 1,2,3-triazol-4-yl]phenyl}-1H-1,2,3-triazol-1- yl)benzoic acid | 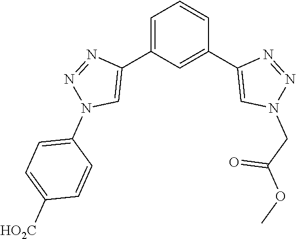 [SMILES: COC(=O)Cn1cc(-c2cccc(-c3cn(-c4ccc(C(=O)O)cc4)nn3)c2)nn1] |
| 178 | 4-[4-({p-[(E)-1-{6-[(E)-1-(p-{[1-(4-Carboxy- 3-hydroxyphenyl)-1H-1,2,3-triazol-4- yl]methoxy}phenylimino)ethyl]-2- pyridyl}ethylideneamino]phenoxy}methyl)- 1H-1,2,3-triazol-1-yl]-2-hydroxybenzoic acid | 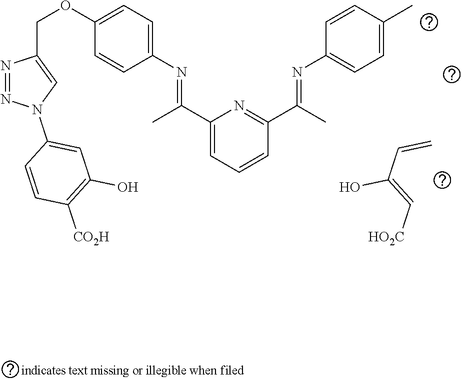 |
| 179 | p-{m-[1-(p-Carboxyphenyl)-1H-1,2,3-triazol- 4-yl]phenylamino} benzoic acid | 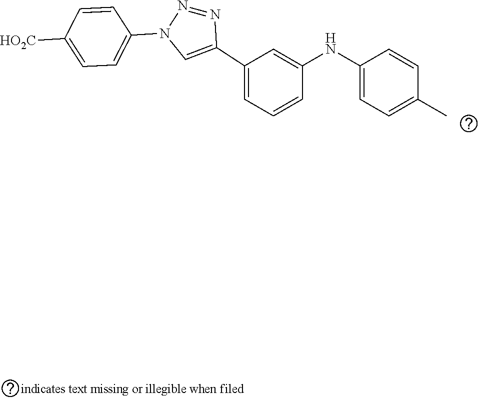 [SMILES: Cc1ccc(Nc2cccc(-c3cn(-c4ccc(C(=O)O)cc4)nn3)c2)cc1] |
| 180 | 4-[4-(Aminomethyl)-1H-1,2,3-triazol-1-yl]- 2-hydroxybenzoic acid | 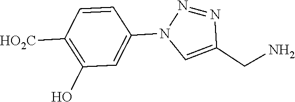 [SMILES: NCc1cn(-c2ccc(C(=O)O)c(O)c2)nn1] |
| 181 | 1-(4-Carboxy-3-hydroxyphenyl)-1H-1,2,3- triazole-4-carboxylic acid | 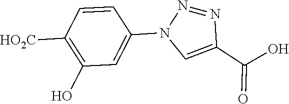 [SMILES: O=C(O)c1cn(-c2ccc(C(=O)O)c(O)c2)nn1] |
| 182 | 2-(4-{6-[1-(1,3-Dicarboxypropyl)-1H-1,2,3- triazol-4-yl]-2-pyridyl}-1H-1,2,3-triazol-1- yl)glutaric acid | 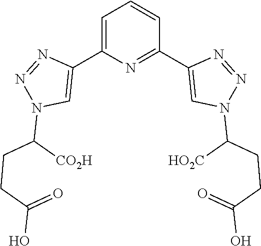 [SMILES: O=C(O)CCC(C(=O)O)n1cc(-c2cccc(-c3cn(C(CCC(=O)O)C(=O)O)nn3)n2)nn1] |
| 183 | 2-(4-{6-[1-(1,2-Dicarboxyethyl)-1H-1,2,3- triazol-4-yl]-2-pyridyl}-1H-1,2,3-triazol-1- yl)succinic acid | 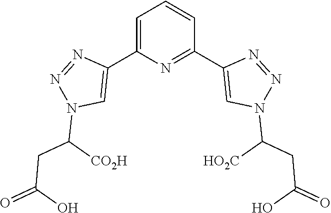 [SMILES: O=C(O)CC(C(=O)O)n1cc(-c2cccc(-c3cn(C(CC(=O)O)C(=O)O)nn3)n2)nn1] |
| 184 | 4-(4-{5-Carboxy-3-[1-(4-carboxy-3- hydroxyphenyl)-1H-1,2,3-triazol-4- yl]phenyl}-1H-1,2,3-triazol-1-yl)-2- hydroxybenzoic acid | 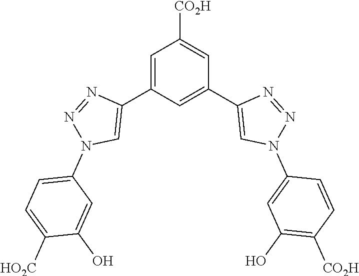 [SMILES: O=C(O)c1cc(-c2cn(-c3ccc(C(=O)O)c(O)c3)nn2)cc(-c2cn(-c3ccc(C(=O)O)c(O)c3)nn2)c1] |
| 185 | 4-(4-{3-Amino-6-[1-(4-carboxy-3- hydroxyphenyl)-1H-1,2,3-triazol-4-yl]-2- pyrazinyl}-1H-1,2,3-triazol-1-yl)-2- hydroxybenzoic acid | 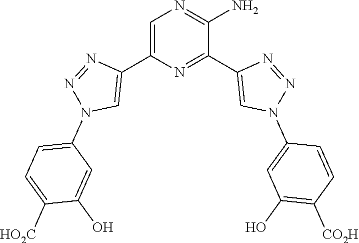 [SMILES: Nc1ncc(-c2cn(-c3ccc(C(=O)O)c(O)c3)nn2)nc1-c1cn(-c2ccc(C(=O)O)c(O)c2)nn1] |
| 186 | 4-(4-{4-Amino-6-[1-(4-carboxy-3- hydroxyphenyl)-1H-1,2,3-triazol-4-yl]-2- pyridyl}-1H-1,2,3-triazol-1-yl)-2- hydroxybenzoic acid | 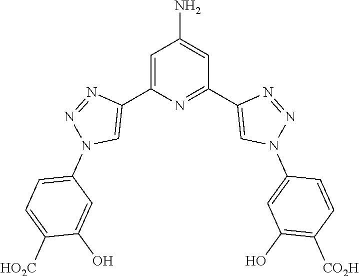 [SMILES: Nc1cc(-c2cn(-c3ccc(C(=O)O)c(O)c3)nn2)nc(-c2cn(-c3ccc(C(=O)O)c(O)c3)nn2)c1] |
| 187 | 4-{1-[(6-{[4-(4-Carboxy-3-hydroxyphenyl)- 1H-1,2,3-triazol-1-yl]methyl}-2- pyridyl)methyl]-1H-1,2,3-triazol-4-yl}-2- hydroxybenzoic acid | 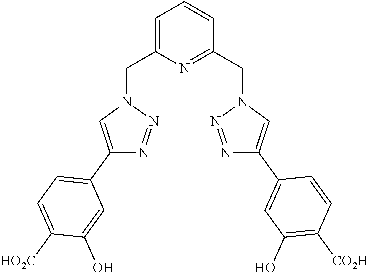 [SMILES: O=C(O)c1ccc(-c2cn(Cc3cccc(Cn4cc(-c5ccc(C(=O)O)c(O)c5)nn4)n3)nn2)cc1O] |
| 188 | 4-{1-[2-(4-{2-[4-(4-Carboxy-3- hydroxyphenyl)-1H-1,2,3-triazol-1-yl]ethyl}- 1-piperazinyl)ethyl]-1H-1,2,3-triazol-4-yl}-2- hydroxybenzoic acid | 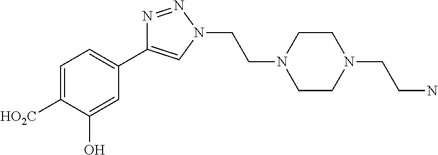 [SMILES: NCCN1CCN(CCn2cc(-c3ccc(C(=O)O)c(O)c3)nn2)CC1] |
| 189 | {1-[2-(4-{2-[4-(Aminomethyl)-1H-1,2,3- triazol-1-yl]ethyl}-1-piperazinyl)ethyl]-1H- 1,2,3-triazol-4-yl} methanamine | 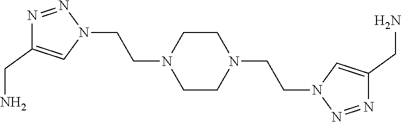 [SMILES: NCc1cn(CCN2CCN(CCn3cc(CN)nn3)CC2)nn1] |
| 190 | 4-{4-[{{[1-(4-Carboxy-3-hydroxyphenyl)- 1H-1,2,3-triazol-4-yl]methyl}amino)methyl]- 1H-1,2,3-triazol-1-yl}-2-hydroxybenzoic acid | 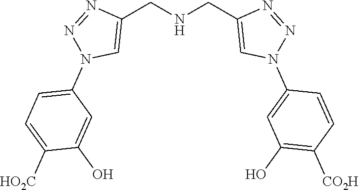 [SMILES: O=C(O)c1ccc(-n2cc(CNCc3cn(-c4ccc(C(=O)O)c(O)c4)nn3)nn2)cc1O] |
| 191 | 5-(4-{6-[1-(3-Carboxy-4-hydroxyphenyl)- 1H-1,2,3-triazol-4-yl]-4-ethoxycarbonyl-2- pyridyl}-1H-1,2,3-triazol-1-yl)-2- hydroxybenzoic acid | 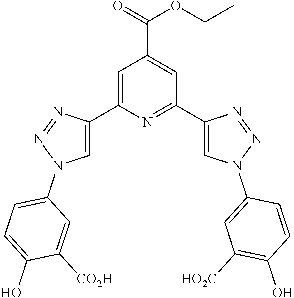 [SMILES: CCOC(=O)c1cc(-c2cn(-c3ccc(O)c(C(=O)O)c3)nn2)nc(-c2cn(-c3ccc(O)c(C(=O)O)c3)nn2)c1] |
| 192 | 5-(4-{6-[1-(3-Carboxy-4-hydroxy-5-tolyl)- 1H-1,2,3-triazol-4-yl]-4-methoxycarbonyl-2- pyridyl}-1H-1,2,3-triazol-1-yl)-2-hydroxy-3- toluic acid | 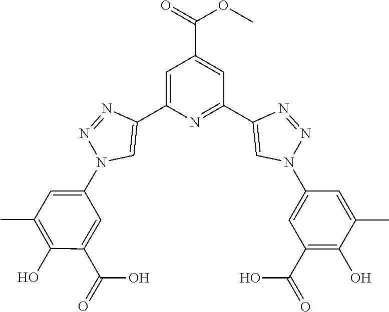 |
| 193 | 4-(4-{6-[1-(4-Carboxy-3-hydroxyphenyl)- 1H-1,2,3-triazol-4-yl]-4-(N,N- dicyclohexylcarbamoyl)-2-pyridyl}-1H- 1,2,3-triazol-1-yl)-2-hydroxybenzoic acid | 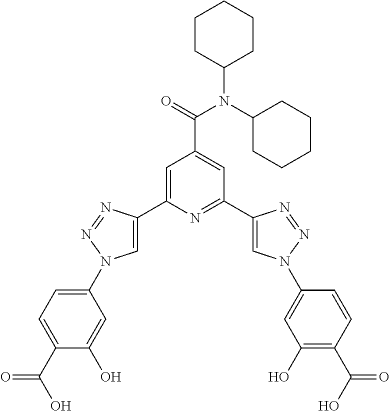 |
| 194 | 4-(4-{6′-[1-(4-Carboxy-3-hydroxyphenyl)- 1H-1,2,3-triazol-4-yl]-2,2′-bipyridyl-6-yl}- 1H-1,2,3-triazol-1-yl)-2-hydroxybenzoic acid | 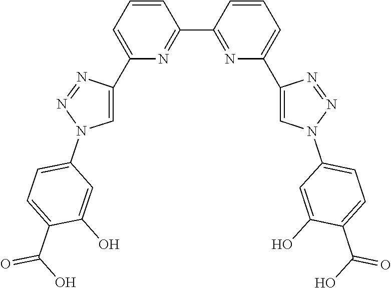 [SMILES: O=C(O)c1ccc(-n2cc(-c3cccc(-c4cccc(-c5cn(-c6ccc(C(=O)O)c(O)c6)nn5)n4)n3)nn2)cc1O] |
| 195 | 4-(4-{7-[1-(4-Carboxy-3-hydroxyphenyl)- 1H-1,2,3-triazol-4-yl]-2-carbazolyl}-1H- 1,2,3-triazol-1-yl)-2-hydroxybenzoic acid | 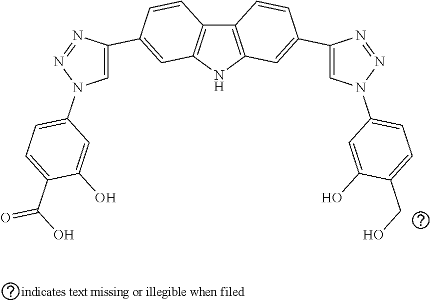 [SMILES: O=C(O)c1ccc(-n2cc(-c3ccc4c(c3)[nH]c3cc(-c5cn(-c6ccc(CO)c(O)c6)nn5)ccc34)nn2)cc1O] |
| 196 | 4-(4-{6″-[1-(4-Carboxy-3-hydroxyphenyl)- 1H-1,2,3-triazol-4-yl]-2,2′:6′,2″-terpyridin-6- yl}-1H-1,2,3-triazol-1-yl)-2-hydroxybenzoic acid | 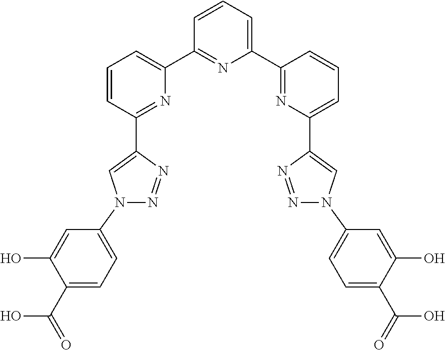 [SMILES: O=C(O)c1ccc(-n2cc(-c3cccc(-c4cccc(-c5cccc(-c6cn(-c7ccc(C(=O)O)c(O)c7)nn6)n5)n4)n3)nn2)cc1O] |
| 197 | N-Ethyl-2,6-bis[1-(3-hydroxypropyl)-1H- 1,2,3-triazol-4-yl]isonicotinamide | 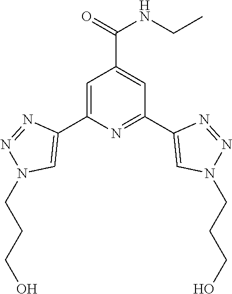 [SMILES: CCNC(=O)c1cc(-c2cn(CCCO)nn2)nc(-c2cn(CCCO)nn2)c1] |
| 198 | 5-(4-{6-[1-(4-Carboxybutyl)-1H-1,2,3- triazol-4-yl]-4-(N-ethylcarbamoyl)-2- pyridyl}-1H-1,2,3-triazol-1-yl)valeric acid | 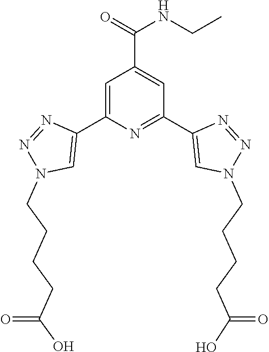 [SMILES: CCNC(=O)c1cc(-c2cn(CCCCC(=O)O)nn2)nc(-c2cn(CCCCC(=O)O)nn2)c1] |
| 199 | (2-{2-[4-(6-{1-[2-(2- Carboxymethoxyethoxy)ethyl]-1H-1,2,3- triazol-4-yl}-4-(N-ethylcarbamoyl)-2- pyridyl)-1H-1,2,3-triazol-1- yl]ethoxy}ethoxy)acetic acid | 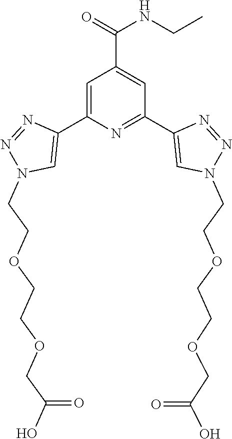 [SMILES: CCNC(=O)c1cc(-c2cn(CCOCCOCC(=O)O)nn2)nc(-c2cn(CCOCCOCC(=O)O)nn2)c1] |
| 200 | N-Ethyl-2,6-bis(1-{2-[2-(2- aminoethoxy)ethoxy]ethyl}-1H-1,2,3-triazol- 4-yl)isonicotinamide | 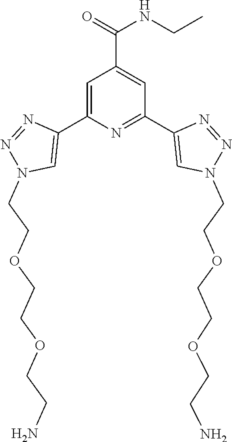 [SMILES: CCNC(=O)c1cc(-c2cn(CCOCCOCCN)nn2)nc(-c2cn(CCOCCOCCN)nn2)c1] |
| 201 | 2-Amino-4-(4-{6-[1-(3-amino-3- carboxypropyl)-1H-1,2,3-triazol-4-yl]-4-(N- ethylcarbamoyl)-2-pyridyl}-1H-1,2,3-triazol- 1-yl)butyric acid | 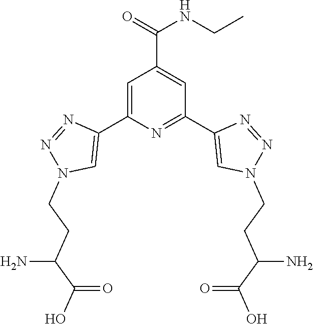 [SMILES: CCNC(=O)c1cc(-c2cn(CCC(N)C(=O)O)nn2)nc(-c2cn(CCC(N)C(=O)O)nn2)c1] |
| 202 | N-Ethyl-2,6-bis[1-(11-hydroxy-3,6,9- trioxaundecyl)-1H-1,2,3-triazol-4- yl]isonicotinamide | 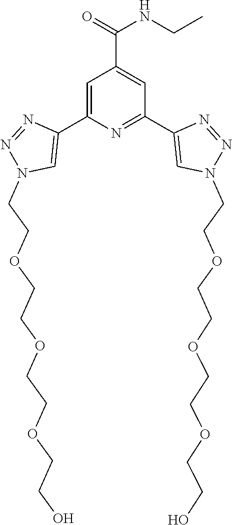 [SMILES: CCNC(=O)c1cc(-c2cn(CCOCCOCCOCCO)nn2)nc(-c2cn(CCOCCOCCOCCO)nn2)c1] |
| 203 | 6-(4-{6-[1-(5-Carboxypentyl)-1H-1,2,3- triazol-4-yl]-4-(N-ethylcarbamoyl)-2- pyridyl}-1H-1,2,3-triazol-1-yl)hexanoic acid | 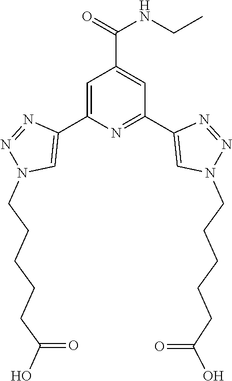 [SMILES: CCNC(=O)c1cc(-c2cn(CCCCCC(=O)O)nn2)nc(-c2cn(CCCCCC(=O)O)nn2)c1] |
| 204 | 4-(4-{6-[1-(5-Carboxy-3-pyrrolidinyl)-1H- 1,2,3-triazol-4-yl]-4-(N-ethylcarbamoyl)-2- pyridyl}-1H-1,2,3-triazol-1-yl)-2- pyrrolidinecarboxylic acid | 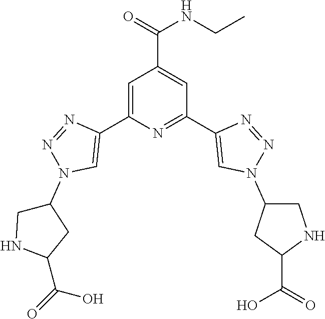 [SMILES: CCNC(=O)c1cc(-c2cn(C3CNC(C(=O)O)C3)nn2)nc(-c2cn(C3CNC(C(=O)O)C3)nn2)c1] |
| 205 | N-Ethyl-2,6-bis[1-(2,4-dioxo-1,2,3,4- tetrahydro-5-pyrimidinyl)-1H-1,2,3-triazol-4- yl]isonicotinamide | 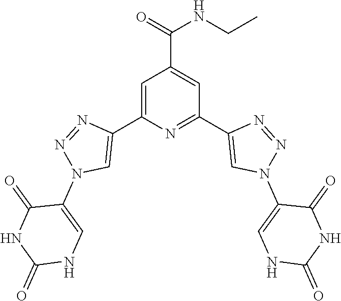 [SMILES: CCNC(=O)c1cc(-c2cn(-c3c[nH]c(=O)[nH]c3=O)nn2)nc(-c2cn(-c3c[nH]c(=O)[nH]c3=O)nn2)c1] |
| 206 | 6-(4-{6-[1-(6-Carboxy-2-naphthyl)-1H-1,2,3- triazol-4-yl]-4-(N-ethylcarbamoyl)-2- pyridyl}-1H-1,2,3-triazol-1-yl)-2-naphthoic acid | 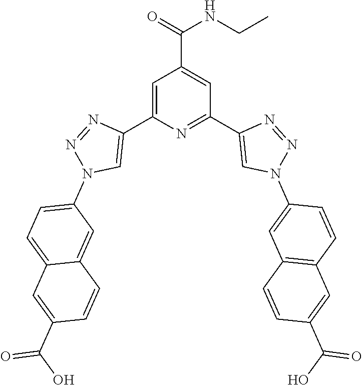 [SMILES: CCNC(=O)c1cc(-c2cn(-c3ccc4cc(C(=O)O)ccc4c3)nn2)nc(-c2cn(-c3ccc4cc(C(=O)O)ccc4c3)nn2)c1] |
| 207 | 4-(4-{6-[1-(2,4-Dicarboxyphenyl)-1H-1,2,3- triazol-4-yl]-4-(N-ethylcarbamoyl)-2- pyridyl}-1H-1,2,3-triazol-1-yl)isophthalic acid | 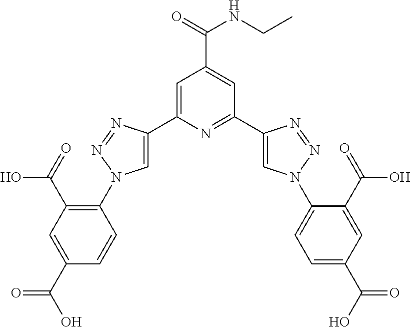 |
| 208 | (4-{6-[1-(Carboxymethyl)-1H-1,2,3-triazol- 4-yl]-4-(N-ethylcarbamoyl)-2-pyridyl}-1H- 1,2,3-triazol-1-yl)acetic acid | 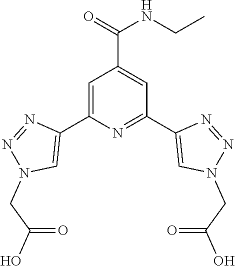 [SMILES: CCNC(=O)c1cc(-c2cn(CC(=O)O)nn2)nc(-c2cn(CC(=O)O)nn2)c1] |
| 209 | N-Ethyl-2,6-bis{1-[2-(7-adenineyl)ethyl]-1H- 1,2,3-triazol-4-yl}isonicotinamide | 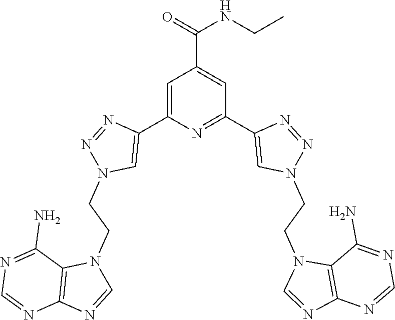 [SMILES: CCNC(=O)c1cc(-c2cn(CCn3cnc4ncnc(N)c43)nn2)nc(-c2cn(CCn3cnc4ncnc(N)c43)nn2)c1] |
| 210 | 2-(4-{6-[1-(2-Carboxy-4-hydroxyphenyl)- 1H-1,2,3-triazol-4-yl]-4-(N-ethylcarbamoyl)- 2-pyridyl}-1H-1,2,3-triazol-1-yl)-5- hydroxybenzoic acid | 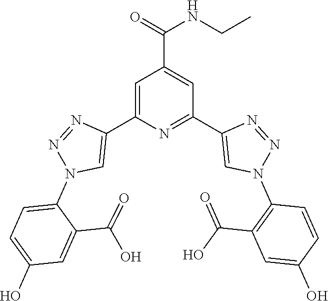 [SMILES: CCNC(=O)c1cc(-c2cn(-c3ccc(O)cc3C(=O)O)nn2)nc(-c2cn(-c3ccc(O)cc3C(=O)O)nn2)c1] |
| 211 | 2-(4-{6-[1-(2-Carboxy-6-hydroxyphenyl)- 1H-1,2,3-triazol-4-yl]-4-(N-ethylcarbamoyl)- 2-pyridyl}-1H-1,2,3-triazol-1-yl)-3- hydroxybenzoic acid | 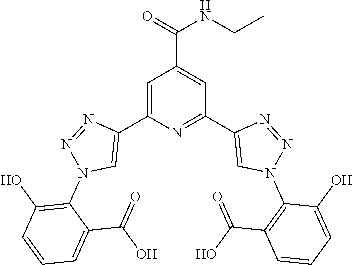 [SMILES: CCNC(=O)c1cc(-c2cn(-c3c(O)cccc3C(=O)O)nn2)nc(-c2cn(-c3c(O)cccc3C(=O)O)nn2)c1] |
| 212 | 3′-(4-{6-[1-(3′-Carboxy-2-hydroxy-3- biphenylyl)-1H-1,2,3-triazol-4-yl]-4-(N- ethylcarbamoyl)-2-pyridyl}-1H-1,2,3-triazol- 1-yl)-2′-hydroxy-3-biphenylcarboxylic acid | 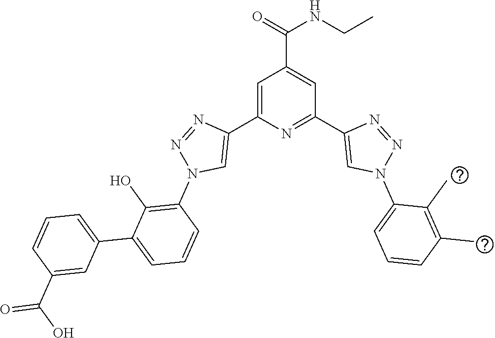 |
| 213 | 3-(4-{6-[1-(3-Carboxy-5-hydroxyphenyl)- 1H-1,2,3-triazol-4-yl]-4-(N-ethylcarbamoyl)- 2-pyridyl}-1H-1,2,3-triazol-1-yl)-5- hydroxybenzoic acid | 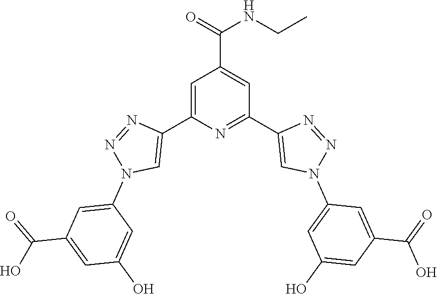 [SMILES: CCNC(=O)c1cc(-c2cn(-c3cc(O)cc(C(=O)O)c3)nn2)nc(-c2cn(-c3cc(O)cc(C(=O)O)c3)nn2)c1] |
| 214 | 4-(4-{6-[1-(4-Carboxy-3-hydroxyphenyl)- 1H-1,2,3-triazol-4-yl]-2-purinyl}-1H-1,2,3- triazol-1-yl)-2-hydroxybenzoic acid | 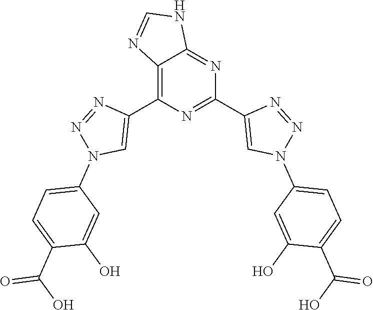 [SMILES: O=C(O)c1ccc(-n2cc(-c3nc(-c4cn(-c5ccc(C(=O)O)c(O)c5)nn4)c4nc[nH]c4n3)nn2)cc1O] |
| 215 | N-Ethyl-2,6-bis{1-[2-(2-amino-6-oxo-1,6- dihydro-7H-purin-7-yl)ethyl]-1H-1,2,3- triazol-4-yl}isonicotinamide | 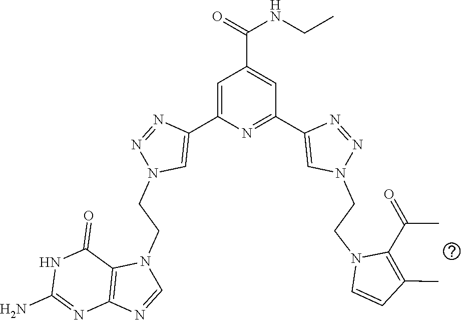 [SMILES: CCNC(=O)c1cc(-c2cn(CCn3ccc(C)c3C(C)=O)nn2)nc(-c2cn(CCn3cnc4nc(N)[nH]c(=O)c43)nn2)c1] |
| 216 | 4-(4-{6-[1-(4-Carboxy-3-hydroxyphenyl)- 1H-1,2,3-triazol-4-yl]-4-(tosylamino)-2- pyridyl}-1H-1,2,3-triazol-1-yl)-2- hydroxybenzoic acid | 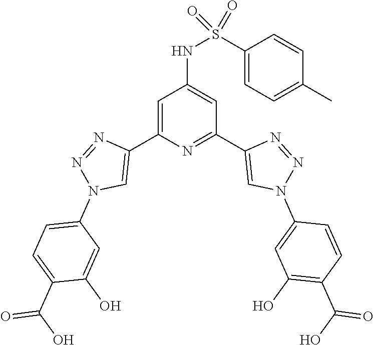 [SMILES: Cc1ccc(S(=O)(=O)Nc2cc(-c3cn(-c4ccc(C(=O)O)c(O)c4)nn3)nc(-c3cn(-c4ccc(C(=O)O)c(O)c4)nn3)c2)cc1] |
| 217 | N-Ethyl-2,6-bis{1-[2-(2,2-dimethyl-1,3- dioxan-5-yloxy)-1-[(2,2-dimethyl-1,3- dioxan-5-yloxy)methyl]ethyl]-1H-1,2,3- triazol-4-yl}isonicotinamide | 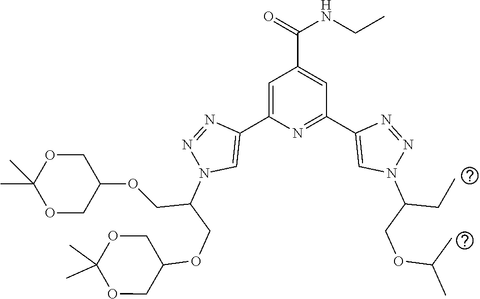 [SMILES: CCNC(=O)c1cc(-c2cn(C(CC)COC(C)C)nn2)nc(-c2cn(C(COC3COC(C)(C)OC3)COC3COC(C)(C)OC3)nn2)c1] |
| 218 | 4-[4-(6-{1-[4-Carboxy-2- (trifluoromethyl)phenyl]-1H-1,2,3-triazol-4- yl}-4-(N-ethylcarbamoyl)-2-pyridyl)-1H- 1,2,3-triazol-1-yl]-3-(trifluoromethyl)benzoic acid | 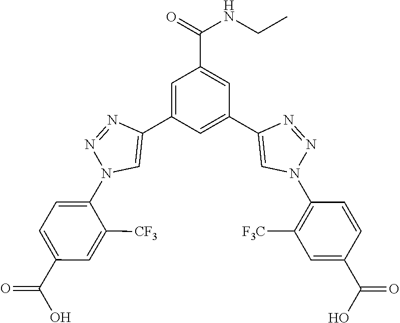 [SMILES: CCNC(=O)c1cc(-c2cn(-c3ccc(C(=O)O)cc3C(F)(F)F)nn2)cc(-c2cn(-c3ccc(C(=O)O)cc3C(F)(F)F)nn2)c1] |
| 219 | 2-[4-(6-{1-[2-Carboxy-5- (trifluoromethyl)phenyl]-1H-1,2,3-triazol-4- yl}-4-(N-ethylcarbamoyl)-2-pyridyl)-1H- 1,2,3-triazol-1-yl]-4-(trifluoromethyl)benzoic acid | 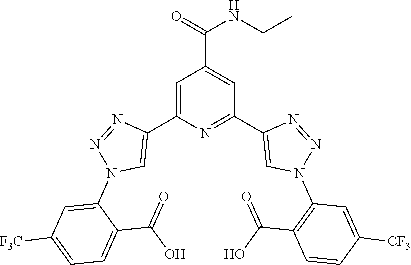 [SMILES: CCNC(=O)c1cc(-c2cn(-c3cc(C(F)(F)F)ccc3C(=O)O)nn2)nc(-c2cn(-c3cc(C(F)(F)F)ccc3C(=O)O)nn2)c1] |
| 220 | 3-[4-(6-{1-[5-Carboxy-2- (trifluoromethyl)phenyl]-1H-1,2,3-triazol-4- yl}-4-(N-ethylcarbamoyl)-2-pyridyl)-1H- 1,2,3-triazol-1-yl]-4-(trifluoromethyl)benzoic acid | 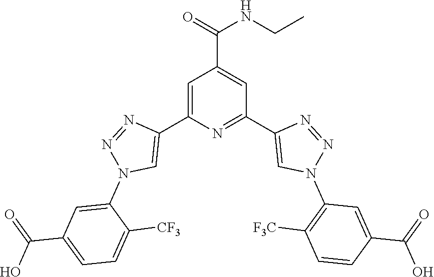 [SMILES: CCNC(=O)c1cc(-c2cn(-c3cc(C(=O)O)ccc3C(F)(F)F)nn2)nc(-c2cn(-c3cc(C(=O)O)ccc3C(F)(F)F)nn2)c1] |
| 221 | N-Ethyl-2,6-bis{1-[m- (trifluoromethyl)phenyl]-1H-1,2,3-triazol-4- yl}isonicotinamide | 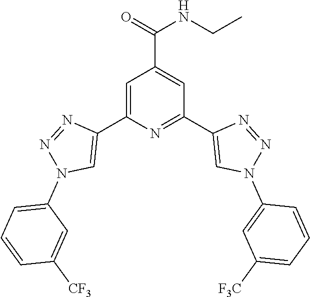 |
| 222 | N-Ethyl-2,6-bis{1-[p- (trifluoromethyl)phenyl]-1H-1,2,3-triazol-4- yl}isonicotinamide | 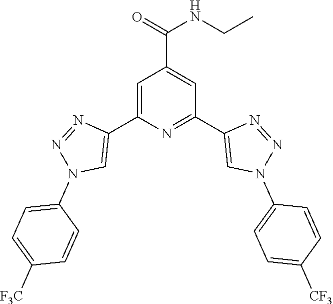 |
| 223 | N-Ethyl-2,6-bis{1-[3,4- bis(trifluoromethyl)phenyl]-1H-1,2,3-triazol- 4-yl}isonicotinamide | 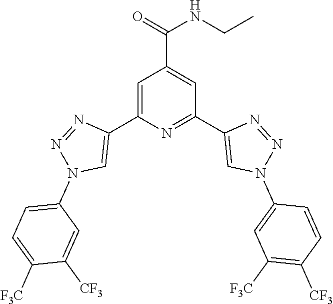 |
| 224 | 4-(4-{6-[1-(4-Carboxy-2,3,5,6- tetrafluorophenyl)-1H-1,2,3-triazol-4-yl]-4- (N-ethylcarbamoyl)-2-pyridyl}-1H-1,2,3- triazol-1-yl)-2,3,5,6-tetrafluorobenzoic acid | 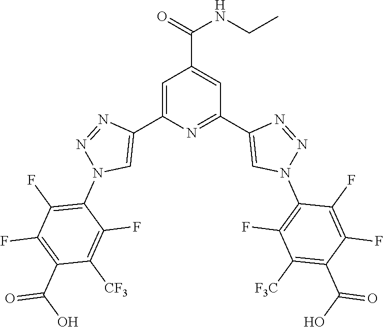 [SMILES: CCNC(=O)c1cc(-c2cn(-c3c(F)c(F)c(C(=O)O)c(C(F)(F)F)c3F)nn2)nc(-c2cn(-c3c(F)c(F)c(C(=O)O)c(C(F)(F)F)c3F)nn2)c1] |
| 225 | 4-(4-{6-[1-(4-Carboxy-3-hydroxyphenyl)- 1H-1,2,3-triazol-4-yl]-4- [(tosylamino)methyl]-2-pyridyl}-1H-1,2,3- triazol-1-yl)-2-hydroxybenzoic acid | 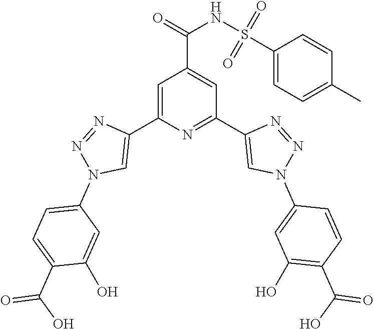 [SMILES: Cc1ccc(S(=O)(=O)NC(=O)c2cc(-c3cn(-c4ccc(C(=O)O)c(O)c4)nn3)nc(-c3cn(-c4ccc(C(=O)O)c(O)c4)nn3)c2)cc1] |
| 226 | N-Ethyl-2,6-bis[1-(3-phosphonopropyl)-1H- 1,2,3-triazol-4-yl]isonicotinamide | 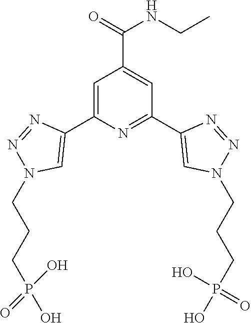 [SMILES: CCNC(=O)c1cc(-c2cn(CCCP(=O)(O)O)nn2)nc(-c2cn(CCCP(=O)(O)O)nn2)c1] |
| 227 | p-(4-{4-[(Hydroxyamino)sulfonyl]-6-[1-(p- sulfophenyl)-1H-1,2,3-triazol-4-yl]-2- pyridyl}-1H-1,2,3λ⁵-triazol-1- yl)benzenesulfonic acid225 | 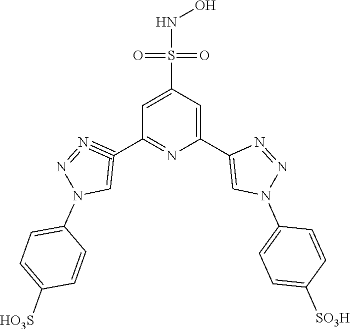 |
| 228 | 4-(4-{6-[1-(2-Carboxy-4-pyridyl)-1H-1,2,3- triazol-4-yl]-4-(N-ethylcarbamoyl)-2- pyridyl}-1H-1,2,3-triazol-1-yl)-2- pyridinecarboxylic acid | 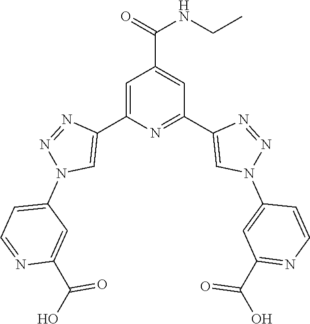 [SMILES: CCNC(=O)c1cc(-c2cn(-c3ccnc(C(=O)O)c3)nn2)nc(-c2cn(-c3ccnc(C(=O)O)c3)nn2)c1] |
| 229 | 5-(4-{6-[1-(2-Carboxy-5-pyrimidinyl)-1H- 1,2,3-triazol-4-yl]-4-(N-ethylcarbamoyl)-2- pyridyl}-1H-1,2,3-triazol-1-yl)-2- pyrimidinecarboxylic acid | 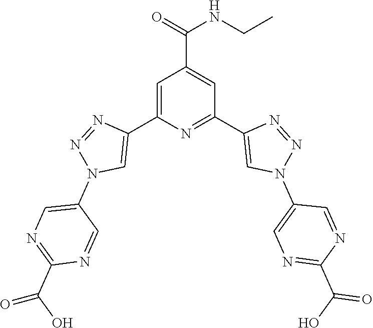 [SMILES: CCNC(=O)c1cc(-c2cn(-c3cnc(C(=O)O)nc3)nn2)nc(-c2cn(-c3cnc(C(=O)O)nc3)nn2)c1] |
| 230 | N-Ethyl-2,6-bis[1-(4,5-diaza-9-phenanthryl)- 1H-1,2,3-triazol-4-yl]isonicotinamide | 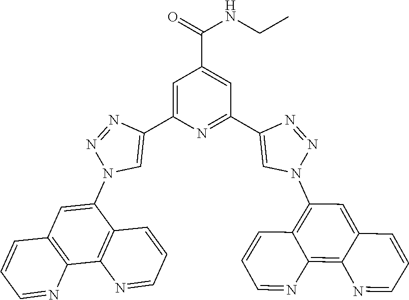 [SMILES: CCNC(=O)c1cc(-c2cn(-c3cc4cccnc4c4ncccc34)nn2)nc(-c2cn(-c3cc4cccnc4c4ncccc34)nn2)c1] |
| 231 | N-Ethyl-2,6-bis[1-(2-pyridyl)-1H-1,2,3- triazol-4-yl]isonicotinamide | 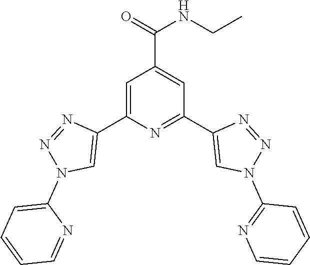 [SMILES: CCNC(=O)c1cc(-c2cn(-c3ccccn3)nn2)nc(-c2cn(-c3ccccn3)nn2)c1] |
| 232 | N-Ethyl-2,6-bis[1-(4-pyridyl)-1H-1,2,3- triazol-4-yl]isonicotinamide | 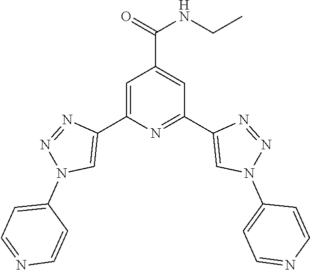 |
| 233 | N-Ethyl-2,6-bis[1-(2-oxo-1,2-dihydro-4- pyridyl)-1H-1,2,3-triazol-4- yl]isonicotinamide | 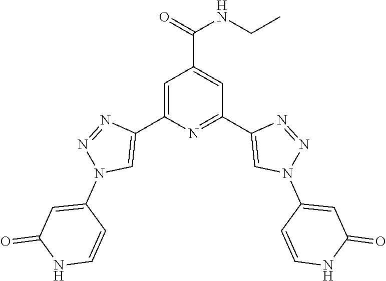 |
| 234 | 4-[4-(6-{1-[4-Carboxy-3- (trifluoromethyl)phenyl]-1H-1,2,3-triazol-4- yl}-4-(N-2-acetylaminoethylcarbamoyl)-2- pyridyl)-1H-1,2,3-triazol-1-yl]-2- (trifluoromethyl)benzoic acid | 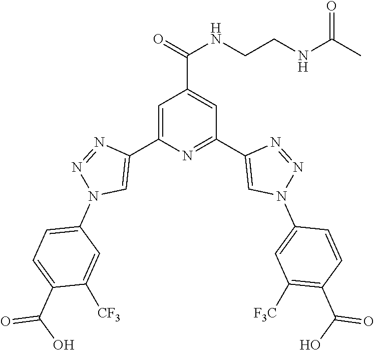 [SMILES: CC(=O)NCCNC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 235 | 4-[1-(6-{4-[4-Carboxy-3- (trifluoromethyl)phenyl]-1H-1,2,3-triazol-1- yl}-2-pyridyl)-1H-1,2,3-triazol-4-yl]-2- (trifluoromethyl)benzoic acid | 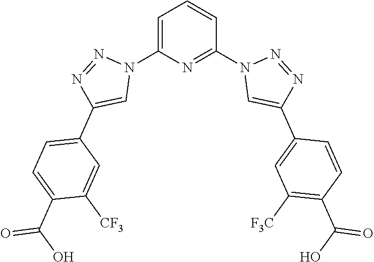 [SMILES: O=C(O)c1ccc(-c2cn(-c3cccc(-n4cc(-c5ccc(C(=O)O)c(C(F)(F)F)c5)nn4)n3)nn2)cc1C(F)(F)F] |
| 236 | N-Ethyl-2,6-bis{1-[4-(N-ethylcarbamoyl)-3- (trifluoromethyl)phenyl]-1H-1,2,3-triazol-4- yl}isonicotinamide | 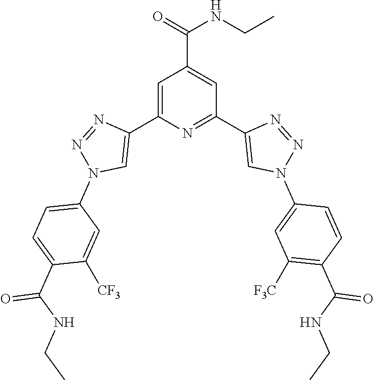 [SMILES: CCNC(=O)c1cc(-c2cn(-c3ccc(C(=O)NCC)c(C(F)(F)F)c3)nn2)nc(-c2cn(-c3ccc(C(=O)NCC)c(C(F)(F)F)c3)nn2)c1] |
| 237 | N-Ethyl-2,6-bis {1-[4-(N-2,2,3,3,4,4,4- heptafluorobutylcarbamoyl)-3- (trifluoromethyl)phenyl]-1H-1,2,3-triazol-4- yl}isonicotinamide | 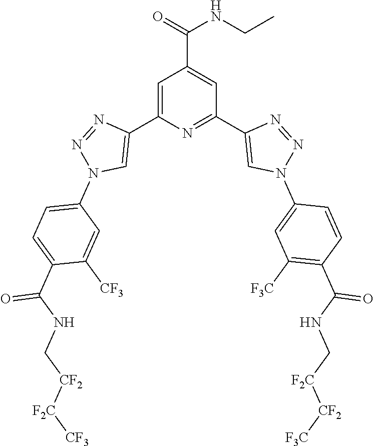 [SMILES: CCNC(=O)c1cc(-c2cn(-c3ccc(C(=O)NCC(F)(F)C(F)(F)C(F)(F)F)c(C(F)(F)F)c3)nn2)nc(-c2cn(-c3ccc(C(=O)NCC(F)(F)C(F)(F)C(F)(F)F)c(C(F)(F)F)c3)nn2)c1] |
| 238 | 4-[4-(2-Amino-3-{1-[4-carboxy-3- (trifluoromethyl)phenyl]-1H-1,2,3-triazol-4- yl}-5-(N-ethylcarbamoyl)phenyl)-1H-1,2,3- triazol-1-yl]-2-(trifluoromethyl)benzoic acid | 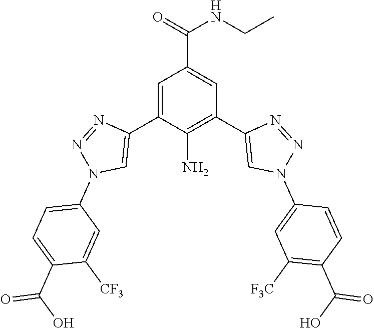 [SMILES: CCNC(=O)c1cc(-c2cn(-c3ccc(C(=O)O)c(C(F)(F)F)c3)nn2)c(N)c(-c2cn(-c3ccc(C(=O)O)c(C(F)(F)F)c3)nn2)c1] |
| 239 | 4-[4-(6-{1-[4-Carboxy-3- (trifluoromethyl)phenyl]-1H-1,2,3-triazol-4- yl}-3-(N-ethylcarbamoyl)-2-pyridyl)-1H- 1,2,3-triazol-1-yl]-2-(trifluoromethyl)benzoic acid | 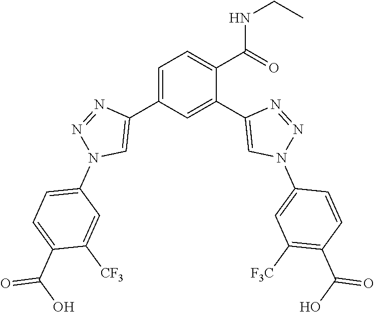 [SMILES: CCNC(=O)c1ccc(-c2cn(-c3ccc(C(=O)O)c(C(F)(F)F)c3)nn2)cc1-c1cn(-c2ccc(C(=O)O)c(C(F)(F)F)c2)nn1] |
| 240 | 4-(4-{4-[6-(1-{4-[N-4-Carboxy-3- (trifluoromethyl)phenylcarbamoyl]-3- (trifluoromethyl)phenyl}-1H-1,2,3-triazol-4- yl)-4-(N-ethylcarbamoyl)-2-pyridyl]-1H- 1,2,3-triazol-1-yl}-2- (trifluoromethyl)benzylamino)-2- (trifluoromethyl)benzoic acid | 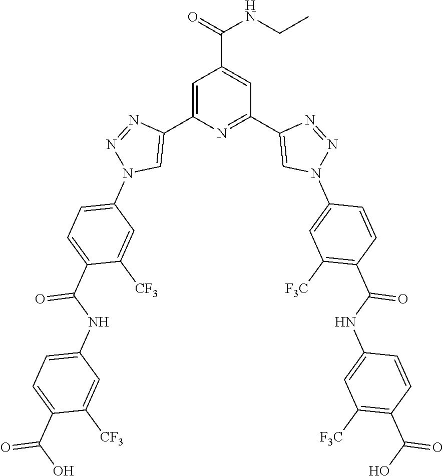 [SMILES: CCNC(=O)c1cc(-c2cn(-c3ccc(C(=O)Nc4ccc(C(=O)O)c(C(F)(F)F)c4)c(C(F)(F)F)c3)nn2)nc(-c2cn(-c3ccc(C(=O)Nc4ccc(C(=O)O)c(C(F)(F)F)c4)c(C(F)(F)F)c3)nn2)c1] |
| 241 | 2-[2-(Trifluoromethyl)-4-{4-[4-(N- ethylcarbamoyl)-6-{1-[4-(N-1,2- dicarboxyethylcarbamoyl)-3- (trifluoromethyl)phenyl]-1H-1,2,3-triazol-4- yl}-2-pyridyl]-1H-1,2,3-triazol-1- yl}benzylamino]succinic acid | 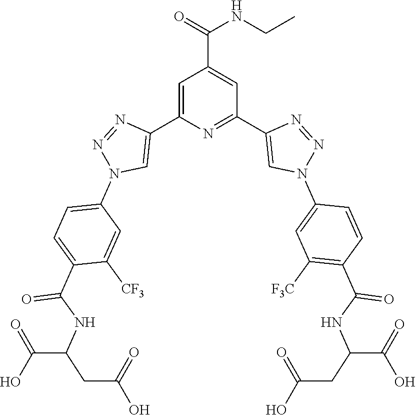 [SMILES: CCNC(=O)c1cc(-c2cn(-c3ccc(C(=O)NC(CC(=O)O)C(=O)O)c(C(F)(F)F)c3)nn2)nc(-c2cn(-c3ccc(C(=O)NC(CC(=O)O)C(=O)O)c(C(F)(F)F)c3)nn2)c1] |
| 242 | 4-[4-(5-{1-[4-Carboxy-3- (trifluoromethyl)phenyl]-1H-1,2,3-triazol-4- yl}-2-pyrrolyl)-1H-1,2,3-triazol-1-yl]-2- (trifluoromethyl)benzoic acid | 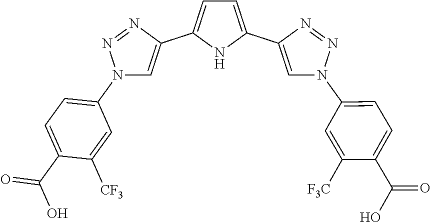 [SMILES: O=C(O)c1ccc(-n2cc(-c3ccc(-c4cn(-c5ccc(C(=O)O)c(C(F)(F)F)c5)nn4)[nH]3)nn2)cc1C(F)(F)F] |
| 243 | 4-[4-(5-{1-[4-Carboxy-3- (trifluoromethyl)phenyl]-1H-1,2,3-triazol-4- yl}-3-ethoxycarbonyl-2-furyl)-1H-1,2,3- triazol-1-yl]-2-(trifluoromethyl)benzoic acid | 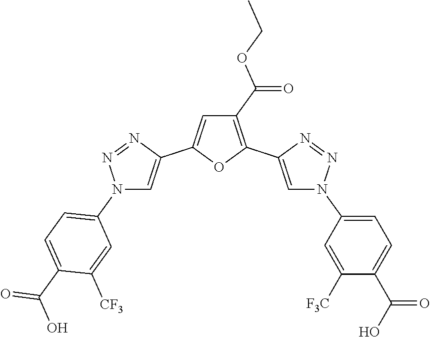 [SMILES: CCOC(=O)c1cc(-c2cn(-c3ccc(C(=O)O)c(C(F)(F)F)c3)nn2)oc1-c1cn(-c2ccc(C(=O)O)c(C(F)(F)F)c2)nn1] |
| 244 | 4-[4-(6-{1-[4-Carboxy-3- (trifluoromethyl)phenyl]-1H-1,2,3-triazol-4- yl}-3,5-dichloro-2-pyridyl)-1H-1,2,3-triazol- 1-yl]-2-(trifluoromethyl)benzoic acid | 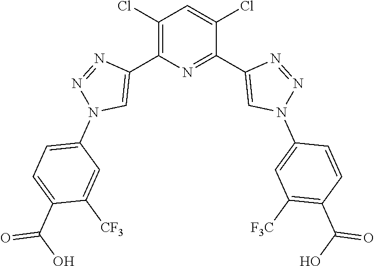 [SMILES: O=C(O)c1ccc(-n2cc(-c3nc(-c4cn(-c5ccc(C(=O)O)c(C(F)(F)F)c5)nn4)c(Cl)cc3Cl)nn2)cc1C(F)(F)F] |
| 245 | 4-(4-{6-[1-(4-Carboxy-3,5-dichlorophenyl)- 1H-1,2,3-triazol-4-yl]-4-(N-ethylcarbamoyl)- 2-pyridyl}-1H-1,2,3-triazol-1-yl)-2,6- dichlorobenzoic acid | 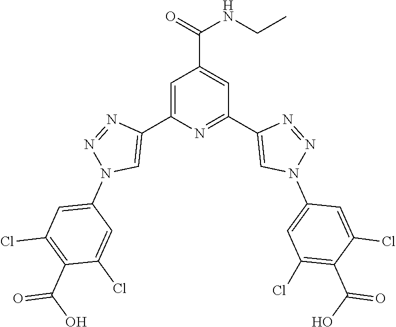 [SMILES: CCNC(=O)c1cc(-c2cn(-c3cc(Cl)c(C(=O)O)c(Cl)c3)nn2)nc(-c2cn(-c3cc(Cl)c(C(=O)O)c(Cl)c3)nn2)c1] |
| 246 | 4-[4-(6-{1-[4-Carboxy-3- (trifluoromethyl)phenyl]-1H-1,2,3-triazol-4- yl}-4-[N-2-(2- methoxyethoxy)ethylcarbamoyl]-2-pyridyl)- 1H-1,2,3-triazol-1-yl]-2- (trifluoromethyl)benzoic acid | 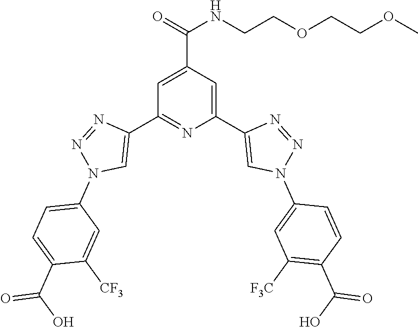 [SMILES: COCCOCCNC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 247 | 4-[4-(6-{1-[4-Carboxy-3- (trifluoromethyl)phenyl]-1H-1,2,3-triazol-4- yl}-4-(N-3,6,9,12,15,18,21,24- octaoxapentacosylcarbamoyl)-2-pyridyl)-1H- 1,2,3-triazol-1-yl]-2-(trifluoromethyl)benzoic acid | 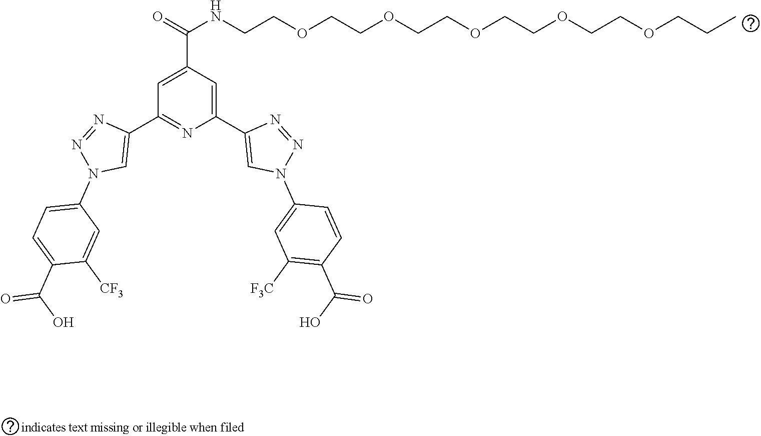 [SMILES: CCCOCCOCCOCCOCCOCCNC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 248 | 4-[4-(6-{1-[4-Carboxy-3- (trifluoromethyl)phenyl]-1H-1,2,3-triazol-4- yl}-4-{N-3-[4-(3- aminopropylamino)butylamino] propylcarbamoyl}-2-pyridyl)- 1H-1,2,3-triazol-1-yl]-2- (trifluoromethyl)benzoic acid | 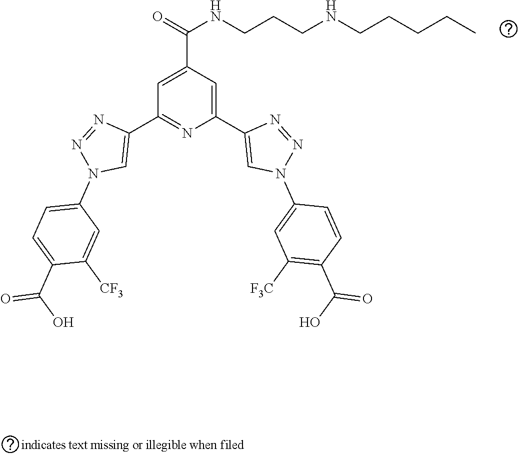 [SMILES: CCCCCNCCCNC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 249 | 4-[4-(6-{1-[4-Carboxy-3- (trifluoromethyl)phenyl]-1H-1,2,3-triazol-4- yl}-4-methoxycarbonyl-2-pyridyl)-1H-1,2,3- triazol-1-yl]-2-(trifluoromethyl)benzoic acid | 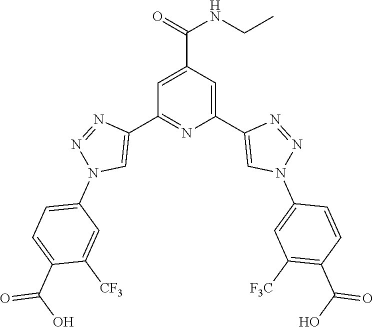 [SMILES: CCNC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 250 | 4-[4-(6-{1-[4-Carboxy-3- (trifluoromethyl)phenyl]-1H-1,2,3-triazol-4- yl}-4-methyl-2-pyridyl)-1H-1,2,3-triazol-1- yl]-2-(trifluoromethyl)benzoic acid | 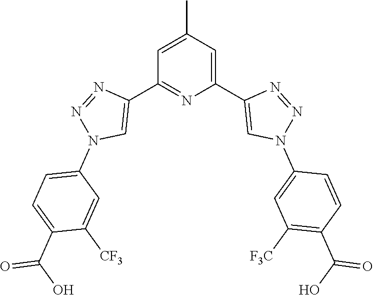 [SMILES: Cc1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 251 | 4-(4-{6-[1-(4-Carboxy-3-hydroxyphenyl)- 1H-1,2,3-triazol-4-yl]-4-(N-3,3,3- trifluoropropylcarbamoyl)-2-pyridyl}-1H- 1,2,3-triazol-1-yl)-2-hydroxybenzoic acid | 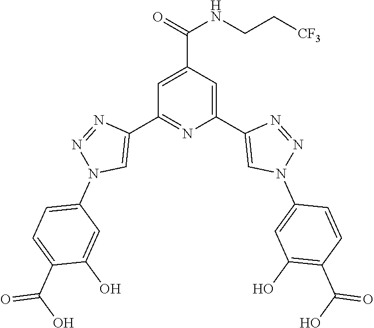 |
| 252 | 4-[4-(6-{1-[4-Carboxy-3- (trifluoromethyl)phenyl]-1H-1,2,3-triazol-4- yl}-4-(N-2-aminoethylcarbamoyl)-2-pyridyl)- 1H-1,2,3-triazol-1-yl]-2- (trifluoromethyl)benzoic acid | 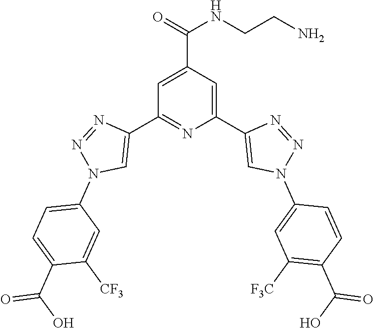 [SMILES: NCCNC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 253 | p-{4-[4-(N-Ethylcarbamoyl)-6-[1-(p- sulfophenyl)-1H-1,2,3-triazol-4-yl]-2- pyridyl]-1H-1,2,3-triazol-1- yl}benzenesulfonic acid | 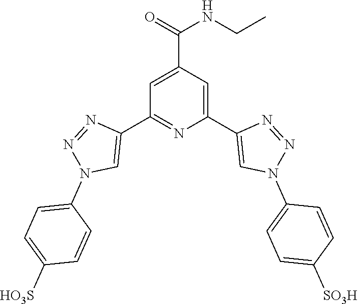 [SMILES: CCNC(=O)c1cc(-c2cn(-c3ccc(S(=O)(=O)O)cc3)nn2)nc(-c2cn(-c3ccc(S(=O)(=O)O)cc3)nn2)c1] |
| 254 | 4-[4-(6-{1-[4-Carboxy-3- (trifluoromethyl)phenyl]-1H-1,2,3-triazol-4- yl}-4-cyano-2-pyridyl)-1H-1,2,3-triazol-1- yl]-2-(trifluoromethyl)benzoic acid | 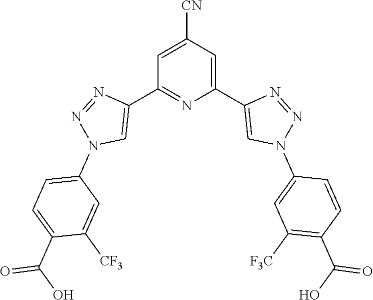 [SMILES: N#Cc1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 255 | 4-[4-(6-{1-[4-Carboxy-3- (trifluoromethyl)phenyl]-1H-1,2,3-triazol-4- yl}-4-(N-acetylcarbamoyl)-2-pyridyl)-1H- 1,2,3-triazol-1-yl]-2-(trifluoromethyl)benzoic acid | 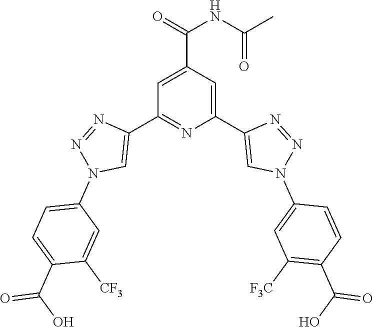 [SMILES: CC(=O)NC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 256 | 4-(4-{6-[1-(4-Carboxy-3-nitrophenyl)-1H- 1,2,3-triazol-4-yl]-4-(N-3,3,3- trifluoropropylcarbamoyl)-2-pyridyl}-1H- 1,2,3-triazol-1-yl)-2-nitrobenzoic acid | 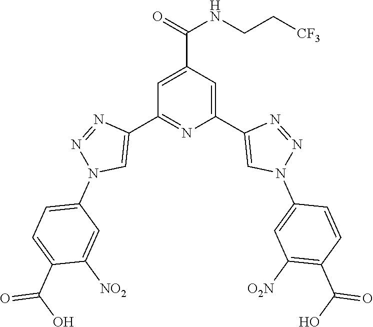 [SMILES: O=C(NCCC(F)(F)F)c1cc(-c2cn(-c3ccc(C(=O)O)c([N+](=O)[O-])c3)nn2)nc(-c2cn(-c3ccc(C(=O)O)c([N+](=O)[O-])c3)nn2)c1] |
| 257 | 4-(4-{6-[1-(4-Carboxy-3-nitrophenyl)-1H- 1,2,3-triazol-4-yl]-4-(N-butylcarbamoyl)-2- pyridyl}-1H-1,2,3-triazol-1-yl)-2- nitrobenzoic acid | 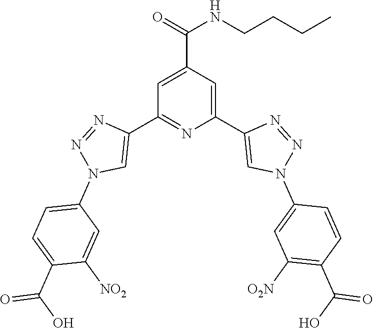 |
| 258 | 4-[4-(6-{1-[4-Carboxy-3- (trifluoromethyl)phenyl]-1H-1,2,3-triazol-4- yl}-3,5-difluoro-4-(N-ethylcarbamoyl)-2- pyridyl)-1H-1,2,3-triazol-1-yl]-2- (trifluoromethyl)benzoic acid | 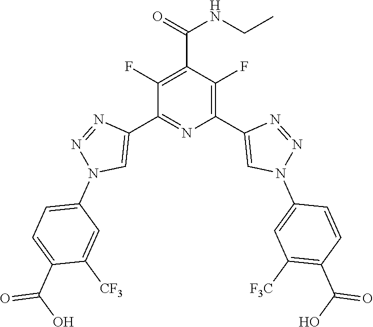 [SMILES: CCNC(=O)c1c(F)c(-c2cn(-c3ccc(C(=O)O)c(C(F)(F)F)c3)nn2)nc(-c2cn(-c3ccc(C(=O)O)c(C(F)(F)F)c3)nn2)c1F] |
| 259 | 4-[4-(6-{1-[4-Carboxy-3- (trifluoromethyl)phenyl]-1H-1,2,3-triazol-4- yl}-4-(ethylaminosulfonyl)-2-pyridyl)-1H- 1,2,3-triazol-1-yl]-2-(trifluoromethyl)benzoic acid | 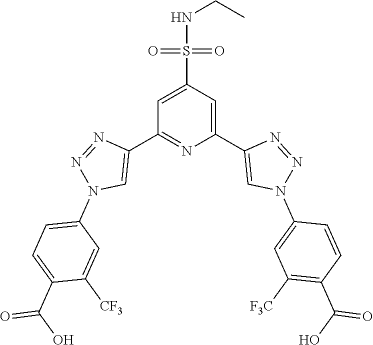 |
| 260 | 2-Nitro-4-{4-[4-(N-ethylcarbamoyl)-6-[1-(3- nitro-4-sulfophenyl)-1H-1,2,3-triazol-4-yl]-2- pyridyl]-1H-1,2,3-triazol-1- yl} benzenesulfonic acid | 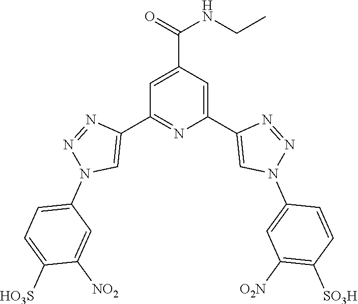 [SMILES: CCNC(=O)c1cc(-c2cn(-c3ccc(S(=O)(=O)O)c([N+](=O)[O-])c3)nn2)nc(-c2cn(-c3ccc(S(=O)(=O)O)c([N+](=O)[O-])c3)nn2)c1] |
| 261 | 4-(4-{6-[1-(4-Carboxy-3-nitrophenyl)-1H- 1,2,3-triazol-4-yl]-4-(N-2- hydroxyethylcarbamoyl)-2-pyridyl}-1H- 1,2,3-triazol-1-yl)-2-nitrobenzoic acid | 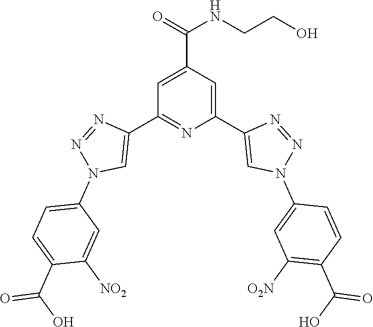 [SMILES: O=C(NCCO)c1cc(-c2cn(-c3ccc(C(=O)O)c([N+](=O)[O-])c3)nn2)nc(-c2cn(-c3ccc(C(=O)O)c([N+](=O)[O-])c3)nn2)c1] |
| 262 | 4-(4-{6-[1-(4-Carboxy-3-nitrophenyl)-1H- 1,2,3-triazol-4-yl]-4-(N- cyclopropylcarbamoyl)-2-pyridyl}-1H-1,2,3- triazol-1-yl)-2-nitrobenzoic acid | 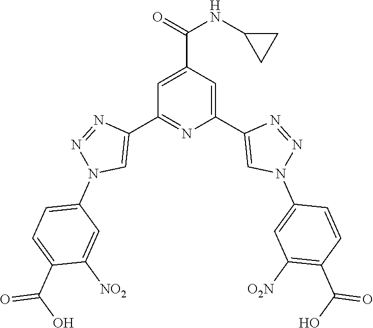 [SMILES: O=C(NC1CC1)c1cc(-c2cn(-c3ccc(C(=O)O)c([N+](=O)[O-])c3)nn2)nc(-c2cn(-c3ccc(C(=O)O)c([N+](=O)[O-])c3)nn2)c1] |
| 263 | 4-(4-{6-[1-(4-Carboxy-3-nitrophenyl)-1H- 1,2,3-triazol-4-yl]-4-methyl-2-pyridyl}-1H- 1,2,3-triazol-1-yl)-2-nitrobenzoic acid | 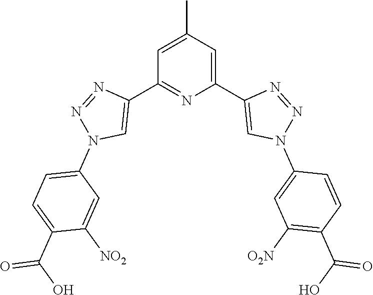 |
| 264 | 4-{4-[4-(Ethylaminosulfonyl)-6-[1-(3-nitro- 4-sulfophenyl)-1H-1,2,3-triazol-4-yl]-2- pyridyl]-1H-1,2,3-triazol-1-yl}-2- nitrobenzenesulfonic acid | 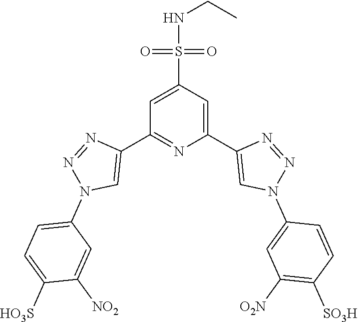 [SMILES: CCNS(=O)(=O)c1cc(-c2cn(-c3ccc(S(=O)(=O)O)c([N+](=O)[O-])c3)nn2)nc(-c2cn(-c3ccc(S(=O)(=O)O)c([N+](=O)[O-])c3)nn2)c1] |
| 265 | N-Ethyl-2,6-bis{ 1-[4-nitro-3- (trifluoromethyl)phenyl]-1H-1,2,3-triazol-4- yl}isonicotinamide | 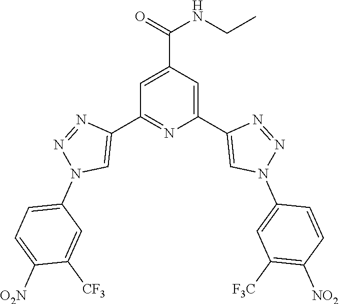 [SMILES: CCNC(=O)c1cc(-c2cn(-c3ccc([N+](=O)[O-])c(C(F)(F)F)c3)nn2)nc(-c2cn(-c3ccc([N+](=O)[O-])c(C(F)(F)F)c3)nn2)c1] |
| 266 | 4-{4-[4-(N-Ethylcarbamoyl)-6-{1-[4-sulfo-3- (trifluoromethyl)phenyl]-1H-1,2,3-triazol-4- yl}-2-pyridyl]-1H-1,2,3-triazol-1-yl}-2- (trifluoromethyl)benzenesulfonic acid | 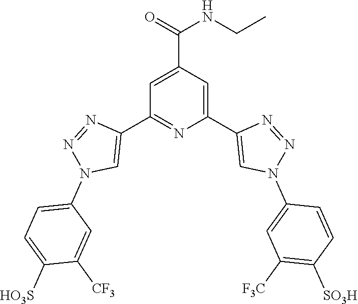 [SMILES: CCNC(=O)c1cc(-c2cn(-c3ccc(S(=O)(=O)O)c(C(F)(F)F)c3)nn2)nc(-c2cn(-c3ccc(S(=O)(=O)O)c(C(F)(F)F)c3)nn2)c1] |
| 267 | 4-(4-{6-[1-(4-Carboxy-3-nitrophenyl)-1H- 1,2,3-triazol-4-yl]-4-(N,N-diethylcarbamoyl)- 2-pyridyl}-1H-1,2,3-triazol-1-yl)-2- nitrobenzoic acid | 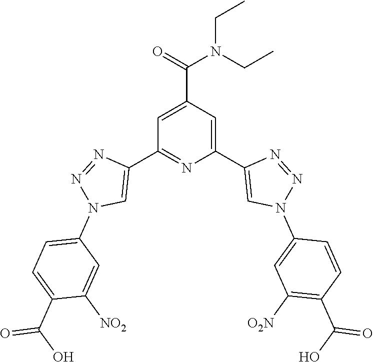 [SMILES: CCN(CC)C(=O)c1cc(-c2cn(-c3ccc(C(=O)O)c([N+](=O)[O-])c3)nn2)nc(-c2cn(-c3ccc(C(=O)O)c([N+](=O)[O-])c3)nn2)c1] |
| 268 | p-{4-[4-(N-Cyclopropylcarbamoyl)-6-[1-(p- sulfophenyl)-1H-1,2,3-triazol-4-yl]-2- pyridyl]-1H-1,2,3-triazol-1- yl} benzenesulfonic acid | 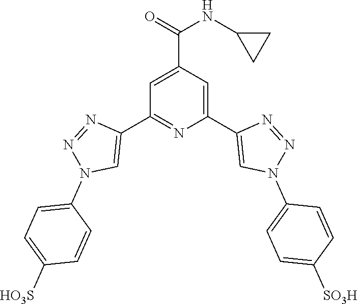 [SMILES: O=C(NC1CC1)c1cc(-c2cn(-c3ccc(S(=O)(=O)O)cc3)nn2)nc(-c2cn(-c3ccc(S(=O)(=O)O)cc3)nn2)c1] |
| 269 | 4-{4-[4-(Ethylaminosulfonyl)-6-{1-[4-sulfo- 3-(trifluoromethyl)phenyl]-1H-1,2,3-triazol- 4-yl}-2-pyridyl]-1H-1,2,3-triazol-1-yl}-2- (trifluoromethyl)benzenesulfonic acid | 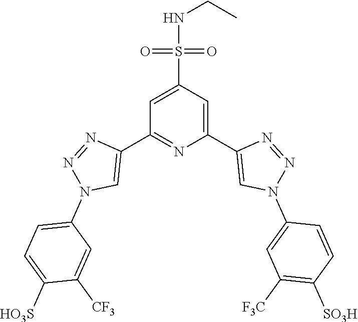 [SMILES: CCNS(=O)(=O)c1cc(-c2cn(-c3ccc(S(=O)(=O)O)c(C(F)(F)F)c3)nn2)nc(-c2cn(-c3ccc(S(=O)(=O)O)c(C(F)(F)F)c3)nn2)c1] |
| 270 | p-{4-[4-(N-3,3,3-Trifluoropropylcarbamoyl)- 6-[1-(p-sulfophenyl)-1H-1,2,3-triazol-4-yl]- 2-pyridyl]-1H-1,2,3-triazol-1- yl} benzenesulfonic acid | 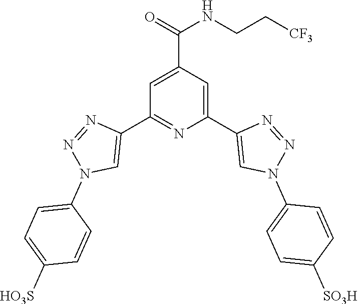 [SMILES: O=C(NCCC(F)(F)F)c1cc(-c2cn(-c3ccc(S(=O)(=O)O)cc3)nn2)nc(-c2cn(-c3ccc(S(=O)(=O)O)cc3)nn2)c1] |
| 271 | 4-[4-(4,6-Bis{ 1-[4-carboxy-3- (trifluoromethyl)phenyl]-1H-1,2,3-triazol-4- yl}-1,3,5-triazin-2-yl)-1H-1,2,3-triazol-1-yl]- 2-(trifluoromethyl)benzoic acid | 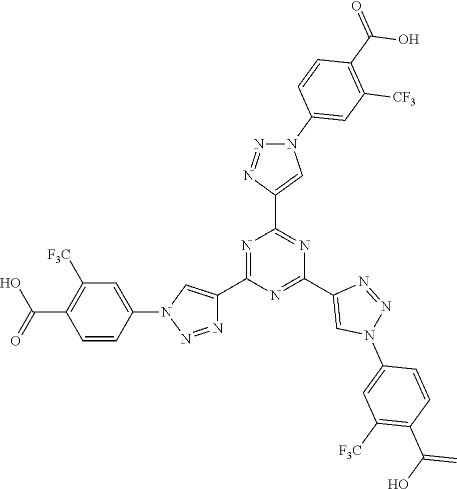 [SMILES: C=C(O)c1ccc(-n2cc(-c3nc(-c4cn(-c5ccc(C(=O)O)c(C(F)(F)F)c5)nn4)nc(-c4cn(-c5ccc(C(=O)O)c(C(F)(F)F)c5)nn4)n3)nn2)cc1C(F)(F)F] |
| 272 | p-(4-{4,6-Bis[1-(p-sulfophenyl)-1H-1,2,3- triazol-4-yl]-1,3,5-triazin-2-yl}-1H-1,2,3- triazol-1-yl)benzenesulfonic acid | 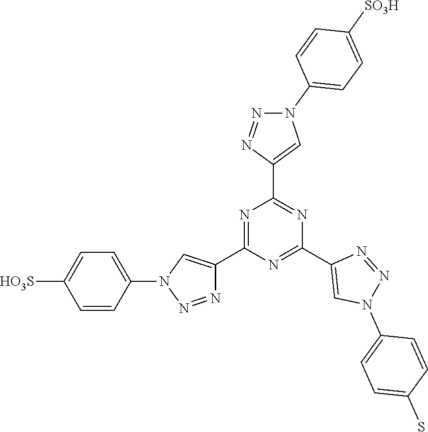 [SMILES: O=S(=O)(O)c1ccc(-n2cc(-c3nc(-c4cn(-c5ccc(S)cc5)nn4)nc(-c4cn(-c5ccc(S(=O)(=O)O)cc5)nn4)n3)nn2)cc1] |
| 273 | p-{4-[4-(Ethylaminosulfonyl)-6-[1-(p- sulfophenyl)-1H-1,2,3-triazol-4-yl]-2- pyridyl]-1H-1,2,3-triazol-1- yl}benzenesulfonic acid | 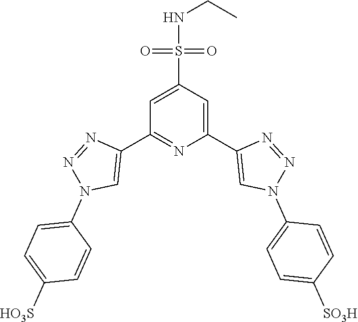 [SMILES: CCNS(=O)(=O)c1cc(-c2cn(-c3ccc(S(=O)(=O)O)cc3)nn2)nc(-c2cn(-c3ccc(S(=O)(=O)O)cc3)nn2)c1] |
| 274 | 4-[4-(6-{1-[4-Carboxy-3- (trifluoromethyl)phenyl]-1H-1,2,3-triazol-4- yl}-4-(diethylaminosulfonyl)-2-pyridyl)-1H- 1,2,3-triazol-1-yl]-2-(trifluoromethyl)benzoic acid | 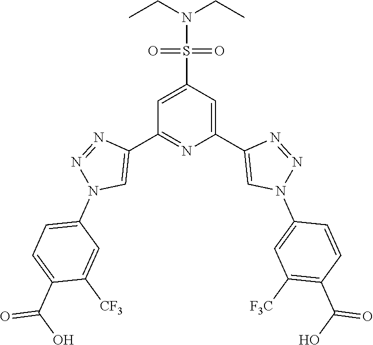 |
| 275 | 4-[4-(6-{1-[4-Carboxy-3- (trifluoromethyl)phenyl]-1H-1,2,3-triazol-4- yl}-2-(N-ethylcarbamoyl)-4-pyridyl)-1H- 1,2,3-triazol-1-yl]-2-(trifluoromethyl)benzoic acid | 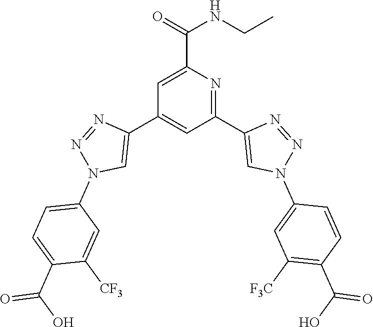 [SMILES: CCNC(=O)c1cc(-c2cn(-c3ccc(C(=O)O)c(C(F)(F)F)c3)nn2)cc(-c2cn(-c3ccc(C(=O)O)c(C(F)(F)F)c3)nn2)n1] |
| 276 | N-Ethyl-2,6-bis{ 1-[3-(trifluoromethyl)-4- {[(trioxidanylphosphino)methyl]carbamoyl} phenyl]-1H-1,2,3-triazol-4-yl}isonicotinamide | 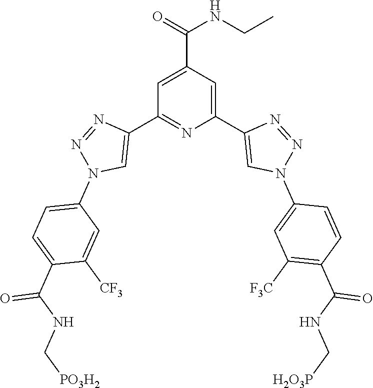 |
| 277 | (4-{4-[4-(N-Ethylcarbamoyl)-6-(1-{4- [(sulfomethyl)carbamoyl]-3- (trifluoromethyl)phenyl}-1H-1,2,3-triazol-4- yl)-2-pyridyl]-1H-1,2,3-triazol-1-yl}-2- (trifluoromethyl)benzylamino) methanesulfonic acid | 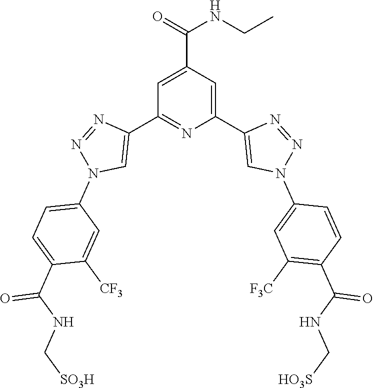 [SMILES: CCNC(=O)c1cc(-c2cn(-c3ccc(C(=O)NCS(=O)(=O)O)c(C(F)(F)F)c3)nn2)nc(-c2cn(-c3ccc(C(=O)NCS(=O)(=O)O)c(C(F)(F)F)c3)nn2)c1] |
| 278 | N-Ethyl-2,6-bis { 1-[p- (dihydroxyboryl)phenyl]-1H-1,2,3-triazol-4- yl}isonicotinamide | 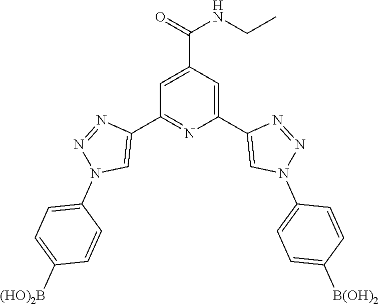 [SMILES: CCNC(=O)c1cc(-c2cn(-c3ccc(B(O)O)cc3)nn2)nc(-c2cn(-c3ccc(B(O)O)cc3)nn2)c1] |
| 279 | N-Ethyl-2,6-bis(1-{p-[2,2,2-trifluoro-1- hydroxy-1-(trifluoromethyl)ethyl]phenyl}- 1H-1,2,3-triazol-4-yl)isonicotinamide | 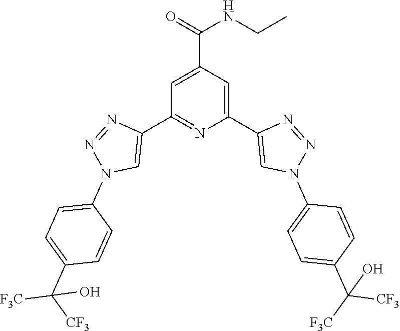 [SMILES: CCNC(=O)c1cc(-c2cn(-c3ccc(C(O)(C(F)(F)F)C(F)(F)F)cc3)nn2)nc(-c2cn(-c3ccc(C(O)(C(F)(F)F)C(F)(F)F)cc3)nn2)c1] |
| 280 | N-Ethyl-2,6-bis{1-[p-(2,2,2-trifluoro-1- hydroxyethyl)phenyl]-1H-1,2,3-triazol-4- yl}isonicotinamide | 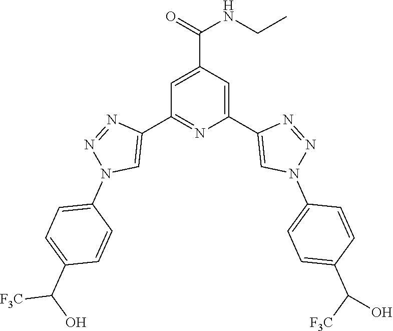 [SMILES: CCNC(=O)c1cc(-c2cn(-c3ccc(C(O)C(F)(F)F)cc3)nn2)nc(-c2cn(-c3ccc(C(O)C(F)(F)F)cc3)nn2)c1] |
| 281 | (S)-1-[4-(4-{6-[1-(4-{[(S)-2-Carboxy-1- pyrrolidinyl]carbonyl}-3- (trifluoromethyl)phenyl)-1H-1,2,3-triazol-4- yl]-4-(N-ethylcarbamoyl)-2-pyridyl}-1H- 1,2,3-triazol-1-yl)-2- (trifluoromethyl)benzoyl]-2- pyrrolidinecarboxylic acid | 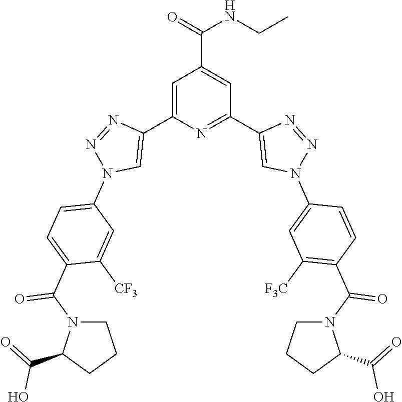 [SMILES: CCNC(=O)c1cc(-c2cn(-c3ccc(C(=O)N4CCC[C@H]4C(=O)O)c(C(F)(F)F)c3)nn2)nc(-c2cn(-c3ccc(C(=O)N4CCC[C@H]4C(=O)O)c(C(F)(F)F)c3)nn2)c1] |
| 282 | (S)-3-(4-Imidazolyl)-2-(4-{4-[6-(1-{4-[N-(S)- 1-carboxy-2-(4-imidazolyl)ethylcarbamoyl]- 3-(trifluoromethyl)phenyl}-1H-1,2,3-triazol- 4-yl)-4-(N-ethylcarbamoyl)-2-pyridyl]-1H- 1,2,3-triazol-1-yl}-2- (trifluoromethyl)benzylamino)propionic acid | 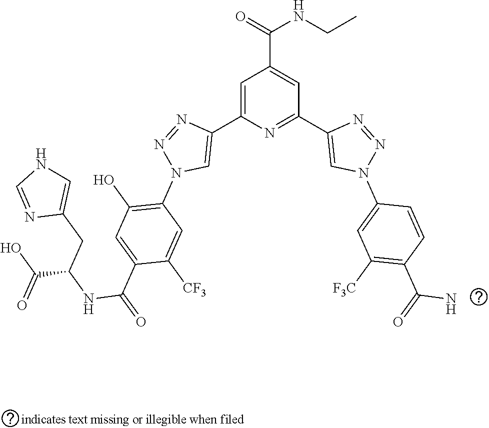 [symbol] |
| 283 | (p-{4-[6-(1-{p- [(Carboxymethyl)aminosulfonyl]phenyl}- 1H-1,2,3-triazol-4-yl)-4-(N-ethylcarbamoyl)- 2-pyridyl]-1H-1,2,3-triazol-1- yl} phenylsulfonylamino)acetic acid | 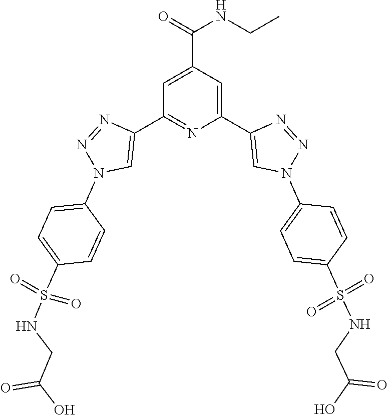 [SMILES: CCNC(=O)c1cc(-c2cn(-c3ccc(S(=O)(=O)NCC(=O)O)cc3)nn2)nc(-c2cn(-c3ccc(S(=O)(=O)NCC(=O)O)cc3)nn2)c1] |
| 284 | (S)-2-(4-{4-[6-(1-{4-[N-(S)-1,3- Dicarboxypropylcarbamoyl]-3- (trifluoromethyl)phenyl}-1H-1,2,3-triazol-4- yl)-4-(N-ethylcarbamoyl)-2-pyridyl]-1H- 1,2,3-triazol-1-yl}-2- (trifluoromethyl)benzylamino)glutaric acid | 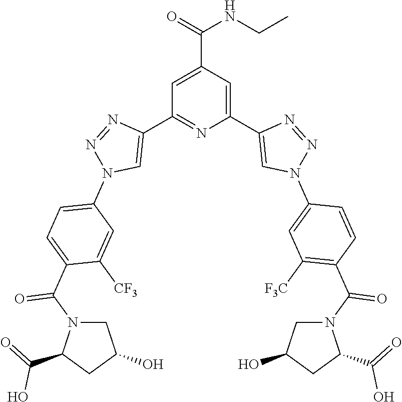 [SMILES: CCNC(=O)c1cc(-c2cn(-c3ccc(C(=O)N4C[C@H](O)C[C@H]4C(=O)O)c(C(F)(F)F)c3)nn2)nc(-c2cn(-c3ccc(C(=O)N4C[C@H](O)C[C@H]4C(=O)O)c(C(F)(F)F)c3)nn2)c1] |
| 285 | (2S,4S)-1-[4-(4-{6-[1-(4-{[(2S,4S)-2- Carboxy-4-hydroxy-1- pyrrolidinyl]carbonyl}-3- (trifluoromethyl)phenyl)-1H-1,2,3-triazol-4- yl]-4-(N-ethylcarbamoyl)-2-pyridyl}-1H- 1,2,3-triazol-1-yl)-2- (trifluoromethyl)benzoyl]-4-hydroxy-2- pyrrolidinecarboxylic acid | 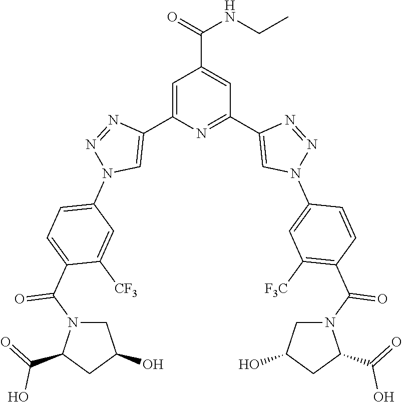 [SMILES: CCNC(=O)c1cc(-c2cn(-c3ccc(C(=O)N4C[C@@H](O)C[C@H]4C(=O)O)c(C(F)(F)F)c3)nn2)nc(-c2cn(-c3ccc(C(=O)N4C[C@@H](O)C[C@H]4C(=O)O)c(C(F)(F)F)c3)nn2)c1] |
| 286 | (2S,4R)-1-[4-(4-{6-[1-(4-{[(2S,4R)-2- Carboxy-4-hydroxy-1- pyrrolidinyl]carbonyl}-3- (trifluoromethyl)phenyl)-1H-1,2,3-triazol-4- yl]-4-(N-ethylcarbamoyl)-2-pyridyl}-1H- 1,2,3-triazol-1-yl)-2- (trifluoromethyl)benzoyl]-4-hydroxy-2- pyrrolidinecarboxylic acid | 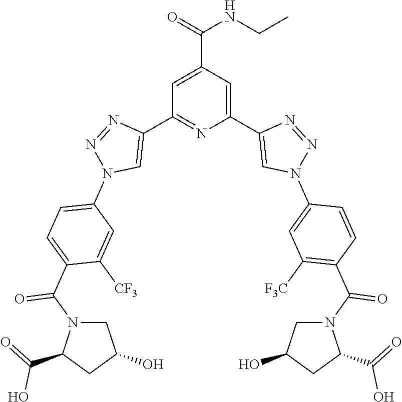 [SMILES: CCNC(=O)c1cc(-c2cn(-c3ccc(C(=O)N4C[C@H](O)C[C@H]4C(=O)O)c(C(F)(F)F)c3)nn2)nc(-c2cn(-c3ccc(C(=O)N4C[C@H](O)C[C@H]4C(=O)O)c(C(F)(F)F)c3)nn2)c1] |
| 287 | 1-(4-{4-[6-(1-{4-[(3-Carboxy-1- azetidinyl)carbonyl]-3- (trifluoromethyl)phenyl}-1H-1,2,3-triazol-4- yl)-4-(N-ethylcarbamoyl)-2-pyridyl]-1H- 1,2,3-triazol-1-yl}-2- (trifluoromethyl)benzoyl)-3- azetidinecarboxylic acid | 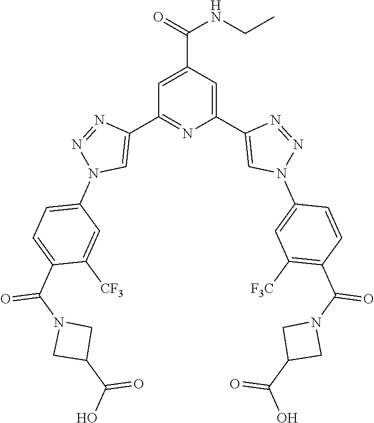 [SMILES: CCNC(=O)c1cc(-c2cn(-c3ccc(C(=O)N4CC(C(=O)O)C4)c(C(F)(F)F)c3)nn2)nc(-c2cn(-c3ccc(C(=O)N4CC(C(=O)O)C4)c(C(F)(F)F)c3)nn2)c1] |
| 288 | 4-[4-(6-{1-[4-Carboxy-3- (trifluoromethyl)phenyl]-1H-1,2,3-triazol-4- yl}-4-[N-2-hydroxy-1- (hydroxymethyl)ethylcarbamoyl]-2-pyridyl)- 1H-1,2,3-triazol-1-yl]-2- (trifluoromethyl)benzoic acid | 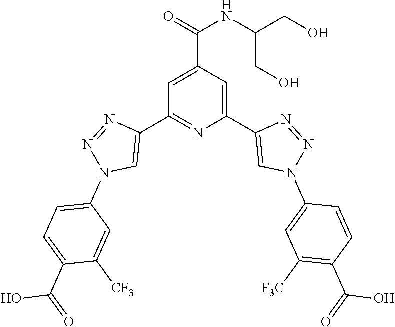 [SMILES: O=C(NC(CO)CO)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 289 | 4-(4-{4-[Bis(2-hydroxyethyl)carbamoyl]-6- {1-[4-carboxy-3-(trifluoromethyl)phenyl]- 1H-1,2,3-triazol-4-yl}-2-pyridyl}-1H-1,2,3- triazol-1-yl)-2-(trifluoromethyl)benzoic acid | 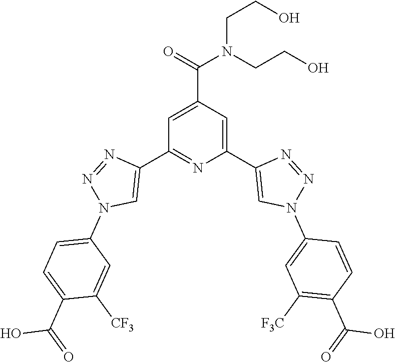 [SMILES: O=C(O)c1ccc(-n2cc(-c3cc(C(=O)N(CCO)CCO)cc(-c4cn(-c5ccc(C(=O)O)c(C(F)(F)F)c5)nn4)n3)nn2)cc1C(F)(F)F] |
| 290 | 4-[4-(5-{1-[4-Carboxy-3- (trifluoromethyl)phenyl]-1H-1,2,3-triazol-4- yl}-3,4-dimethoxy-2-thienyl)-1H-1,2,3- triazol-1-yl]-2-(trifluoromethyl)benzoic acid | 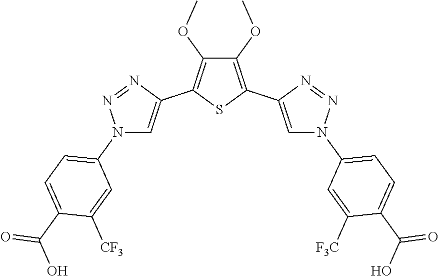 [SMILES: COc1c(-c2cn(-c3ccc(C(=O)O)c(C(F)(F)F)c3)nn2)sc(-c2cn(-c3ccc(C(=O)O)c(C(F)(F)F)c3)nn2)c1OC] |
| 291 | 4-[4-(6-{1-[4-Carboxy-3- (trifluoromethyl)phenyl]-1H-1,2,3-triazol-4- yl}-4-(N-allylcarbamoyl)-2-pyridyl)-1H- 1,2,3-triazol-1-yl]-2-(trifluoromethyl)benzoic acid | 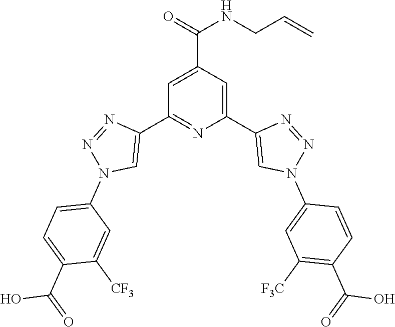 [SMILES: C=CCNC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 292 | 4-[4-(6-{1-[4-Carboxy-3- (trifluoromethyl)phenyl]-1H-1,2,3-triazol-4- yl}-4-({[4-(hydroxymethyl)-1H-1,2,3-triazol- 1-yl]methyl}carbamoyl)-2-pyridyl)-1H-1,2,3- triazol-1-yl]-2-(trifluoromethyl)benzoic acid | 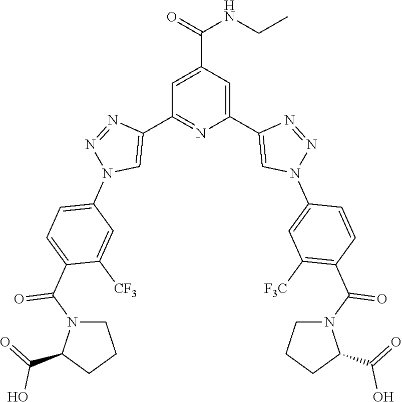 [SMILES: CCNC(=O)c1cc(-c2cn(-c3ccc(C(=O)N4CCC[C@H]4C(=O)O)c(C(F)(F)F)c3)nn2)nc(-c2cn(-c3ccc(C(=O)N4CCC[C@H]4C(=O)O)c(C(F)(F)F)c3)nn2)c1] |
| 293 | 4-[4-(5-{1-[4-Carboxy-3- (trifluoromethyl)phenyl]-1H-1,2,3-triazol-4- yl}-3,4-dimethoxy-2-furyl)-1H-1,2,3-triazol- 1-yl]-2-(trifluoromethyl)benzoic acid | 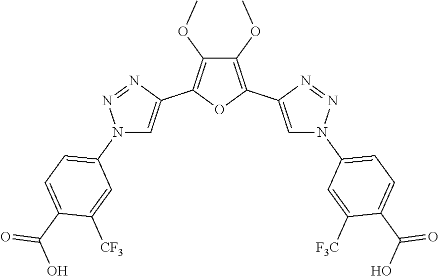 [SMILES: COc1c(-c2cn(-c3ccc(C(=O)O)c(C(F)(F)F)c3)nn2)oc(-c2cn(-c3ccc(C(=O)O)c(C(F)(F)F)c3)nn2)c1OC] |
| 294 | 4-[4-(6-{1-[4-Carboxy-3- (trifluoromethyl)phenyl]-1H-1,2,3-triazol-4- yl}-4-(2-hydroxyethylaminosulfonyl)-2- pyridyl)-1H-1,2,3-triazol-1-yl]-2- (trifluoromethyl)benzoic acid | 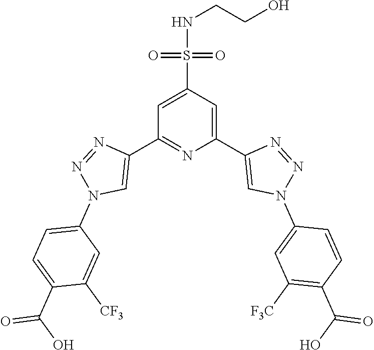 [SMILES: O=C(O)c1ccc(-n2cc(-c3cc(S(=O)(=O)NCCO)cc(-c4cn(-c5ccc(C(=O)O)c(C(F)(F)F)c5)nn4)n3)nn2)cc1C(F)(F)F] |
| 295 | 4-(4-{6-[1-(4-Carboxy-3-hydroxyphenyl)- 1H-1,2,3-triazol-4-yl]-4-(2- hydroxyethylaminosulfonyl)-2-pyridyl}-1H- 1,2,3-triazol-1-yl)-2-hydroxybenzoic acid | 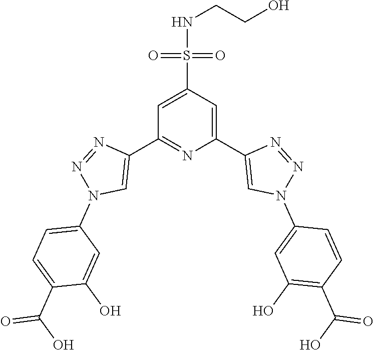 |
| 296 | 4-[4-(5-{1-[4-Carboxy-3- (trifluoromethyl)phenyl]-1H-1,2,3-triazol-4- yl}-3,4-dicyano-2-thienyl)-1H-1,2,3-triazol- 1-yl]-2-(trifluoromethyl)benzoic acid | 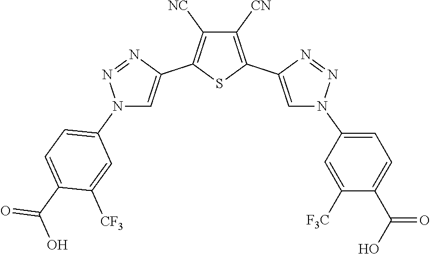 [SMILES: N#Cc1c(-c2cn(-c3ccc(C(=O)O)c(C(F)(F)F)c3)nn2)sc(-c2cn(-c3ccc(C(=O)O)c(C(F)(F)F)c3)nn2)c1C#N] |
| 297 | 4-[4-(8-{1-[4-Carboxy-3- (trifluoromethyl)phenyl]-1H-1,2,3-triazol-4- yl}-9-oxa-1-fluorenyl)-1H-1,2,3-triazol-1- yl]-2-(trifluoromethyl)benzoic acid | 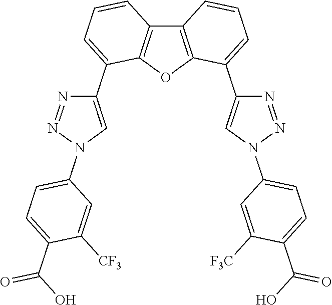 [SMILES: O=C(O)c1ccc(-n2cc(-c3cccc4c3oc3c(-c5cn(-c6ccc(C(=O)O)c(C(F)(F)F)c6)nn5)cccc34)nn2)cc1C(F)(F)F] |
| 298 | 4-[4-(4-{1-[4-Carboxy-3- (trifluoromethyl)phenyl]-1H-1,2,3-triazol-4- yl}-3-furyl)-1H-1,2,3-triazol-1-yl]-2- (trifluoromethyl)benzoic acid | 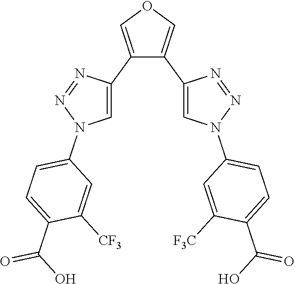 [SMILES: O=C(O)c1ccc(-n2cc(-c3cocc3-c3cn(-c4ccc(C(=O)O)c(C(F)(F)F)c4)nn3)nn2)cc1C(F)(F)F] |
| 299 | 2-[2-(Trifluoromethyl)-4-{4-[4-(N- ethylcarbamoyl)-6-{1-[4-(N-2- sulfoethylcarbamoyl)-3- (trifluoromethyl)phenyl]-1H-1,2,3-triazol-4- yl}-2-pyridyl]-1H-1,2,3-triazol-1- yl} benzylamino]ethanesulfonic acid | 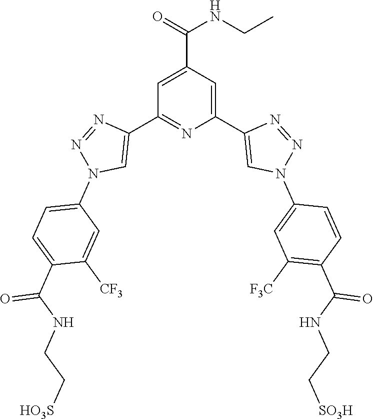 [SMILES: CCNC(=O)c1cc(-c2cn(-c3ccc(C(=O)NCCS(=O)(=O)O)c(C(F)(F)F)c3)nn2)nc(-c2cn(-c3ccc(C(=O)NCCS(=O)(=O)O)c(C(F)(F)F)c3)nn2)c1] |
| 300 | 2-Nitro-4-(4-{5-[1-(3-nitro-4-sulfophenyl)- 1H-1,2,3-triazol-4-yl]-2-furyl}-1H-1,2,3- triazol-1-yl)benzenesulfonic acid | 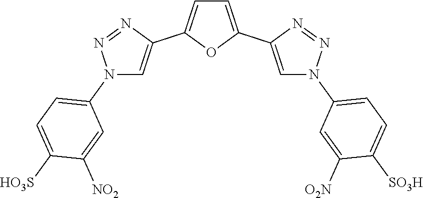 [SMILES: O=[N+]([O-])c1cc(-n2cc(-c3ccc(-c4cn(-c5ccc(S(=O)(=O)O)c([N+](=O)[O-])c5)nn4)o3)nn2)ccc1S(=O)(=O)O] |
| 301 | p-(4-{5-[1-(p-Sulfophenyl)-1H-1,2,3-triazol- 4-yl]-2-furyl}-1H-1,2,3-triazol-1- yl)benzenesulfonic acid | 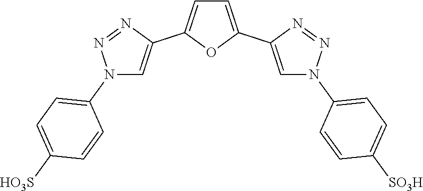 [SMILES: O=S(=O)(O)c1ccc(-n2cc(-c3ccc(-c4cn(-c5ccc(S(=O)(=O)O)cc5)nn4)o3)nn2)cc1] |
| 302 | 4-(4-{5-[1-(4-Carboxy-3-nitrophenyl)-1H- 1,2,3-triazol-4-yl]-2-furyl}-1H-1,2,3-triazol- 1-yl)-2-nitrobenzoic acid | 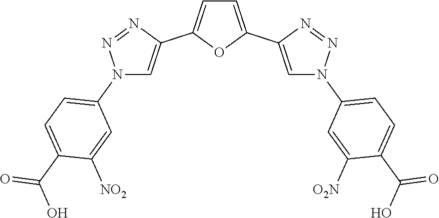 [SMILES: O=C(O)c1ccc(-n2cc(-c3ccc(-c4cn(-c5ccc(C(=O)O)c([N+](=O)[O-])c5)nn4)o3)nn2)cc1[N+](=O)[O-]] |
| 303 | 4-[(E)-5-[(E)-4-Carboxy-3- (trifluoromethyl)phenylazo]-2-furylazo]-2- (trifluoromethyl)benzoic acid | 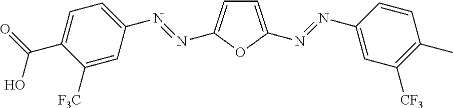 [SMILES: Cc1ccc(/N=N/c2ccc(/N=N/c3ccc(C(=O)O)c(C(F)(F)F)c3)o2)cc1C(F)(F)F] |
| 304 | 4-[(E)-6-[(E)-4-Carboxy-3- (trifluoromethyl)phenylazo]-2-pyridylazo]-2- (trifluoromethyl)benzoic acid | 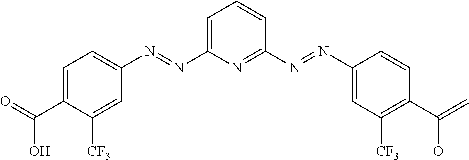 [SMILES: C=C(O)c1ccc(/N=N/c2cccc(/N=N/c3ccc(C(=O)O)c(C(F)(F)F)c3)n2)cc1C(F)(F)F] |
| 305 | 4-[4-(5-{1-[4-Sulfo-3- (trifluoromethyl)phenyl]-1H-1,2,3-triazol-4- yl}-2-furyl)-1H-1,2,3-triazol-1-yl]-2- (trifluoromethyl)benzenesulfonic acid | 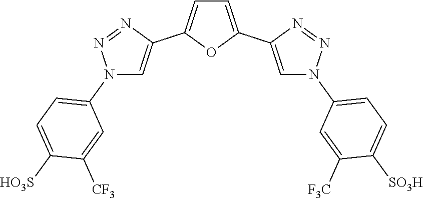 [SMILES: O=S(=O)(O)c1ccc(-n2cc(-c3ccc(-c4cn(-c5ccc(S(=O)(=O)O)c(C(F)(F)F)c5)nn4)o3)nn2)cc1C(F)(F)F] |
| 306 | 4-[4-(6-{1-[4-Carboxy-3- (trifluoromethyl)phenyl]-1H-1,2,3-triazol-4- yl}-4-(trifluoromethyl)-2-pyridyl)-1H-1,2,3- triazol-1-yl]-2-(trifluoromethyl)benzoic acid | 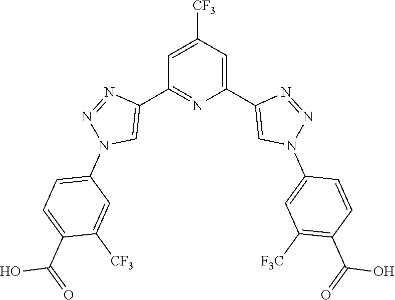 [SMILES: O=C(O)c1ccc(-n2cc(-c3cc(C(F)(F)F)cc(-c4cn(-c5ccc(C(=O)O)c(C(F)(F)F)c5)nn4)n3)nn2)cc1C(F)(F)F] |
| 307 | 4-{4-[(Z)-2-{1-[4-Carboxy-3- (trifluoromethyl)phenyl]-1H-1,2,3-triazol-4- yl}ethenyl]-1H-1,2,3-triazol-1-yl}-2- (trifluoromethyl)benzoic acid | 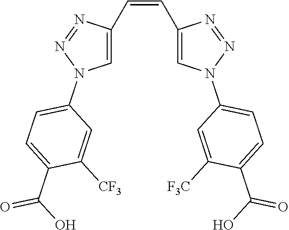 [SMILES: O=C(O)c1ccc(-n2cc(/C=C\c3cn(-c4ccc(C(=O)O)c(C(F)(F)F)c4)nn3)nn2)cc1C(F)(F)F] |
| 308 | 4-(4-{6-[1-(4-Carboxy-3-nitrophenyl)-1H- 1,2,3-triazol-4-yl]-4-(ethylaminosulfonyl)-2- pyridyl}-1H-1,2,3-triazol-1-yl)-2- nitrobenzoic acid | 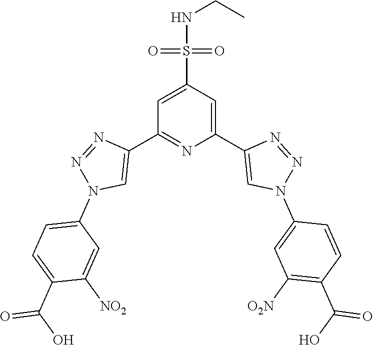 |
| 309 | 4-(4-{6-[1-(4-Carboxy-3-nitrophenyl)-1H- 1,2,3-triazol-4-yl]-4-(trifluoromethyl)-2- pyridyl}-1H-1,2,3-triazol-1-yl)-2- nitrobenzoic acid | 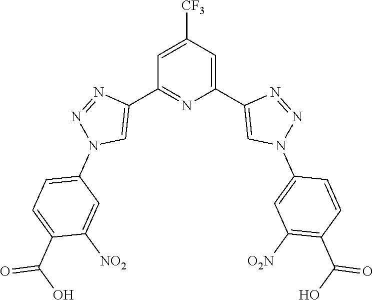 |
| 310 | 4-[4-(6-{1-[4-Carboxy-3- (trifluoromethyl)phenyl]-1H-1,2,3-triazol-4- yl}-4-methoxy-2-pyridyl)-1H-1,2,3-triazol-1- yl]-2-(trifluoromethyl)benzoic acid | 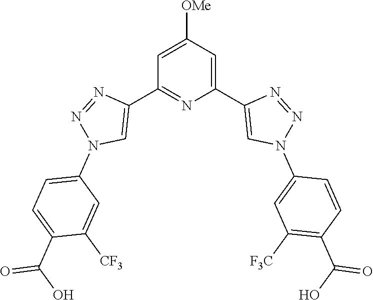 [SMILES: COc1cc(-c2cn(-c3ccc(C(=O)O)c(C(F)(F)F)c3)nn2)nc(-c2cn(-c3ccc(C(=O)O)c(C(F)(F)F)c3)nn2)c1] |
| 311 | 4-(4-{6-[1-(4-Carboxy-3-nitrophenyl)-1H- 1,2,3-triazol-4-yl]-4-methoxy-2-pyridyl}-1H- 1,2,3-triazol-1-yl)-2-nitrobenzoic acid | 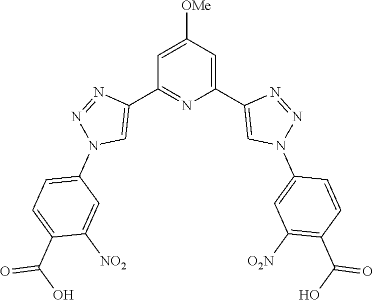 |
| 312 | p-{4-[4-(N-2-Hydroxyethylcarbamoyl)-6-[1- (p-sulfophenyl)-1H-1,2,3-triazol-4-yl]-2- pyridyl]-1H-1,2,3-triazol-1- yl}benzenesulfonic acid | 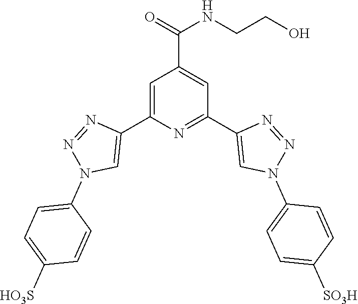 |
| 313 | 4-(4-{1-[4-Carboxy-3- (trifluoromethyl)phenyl]-1H-1,2,3-triazol-4- yl}-1H-1,2,3-triazol-1-yl)-2- (trifluoromethyl)benzoic acid | 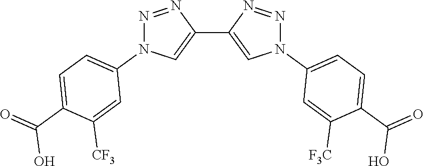 [SMILES: O=C(O)c1ccc(-n2cc(-c3cn(-c4ccc(C(=O)O)c(C(F)(F)F)c4)nn3)nn2)cc1C(F)(F)F] |
| 314 | 4-(4-{5-[1-(4-Carboxy-3-fluorophenyl)-1H- 1,2,3-triazol-4-yl]-2-furyl}-1H-1,2,3-triazol- 1-yl)-2-fluorobenzoic acid | 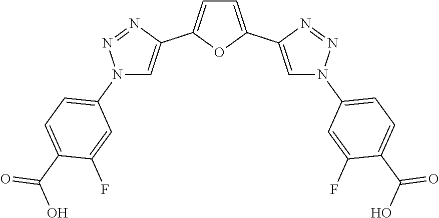 [SMILES: O=C(O)c1ccc(-n2cc(-c3ccc(-c4cn(-c5ccc(C(=O)O)c(F)c5)nn4)o3)nn2)cc1F] |
| 315 | 4-(4-{5-[1-(4-Carboxy-3-cyanophenyl)-1H- 1,2,3-triazol-4-yl]-2-furyl}-1H-1,2,3-triazol- 1-yl)-2-cyanobenzoic acid | 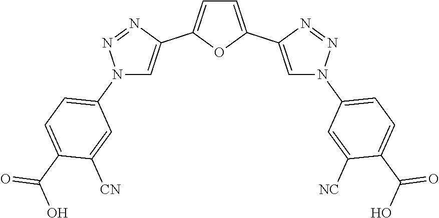 [SMILES: N#Cc1cc(-n2cc(-c3ccc(-c4cn(-c5ccc(C(=O)O)c(C#N)c5)nn4)o3)nn2)ccc1C(=O)O] |
| 316 | 4-(4-{5-[1-(m-Sulfophenyl)-1H-1,2,3-triazol- 4-yl]-2-furyl}-1H-1,2,3-triazol-1-yl)-2- (trifluoromethyl)benzoic acid | 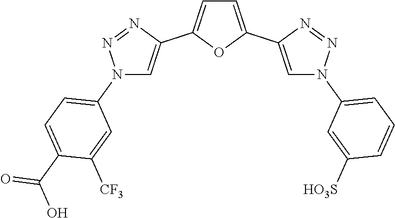 [SMILES: O=C(O)c1ccc(-n2cc(-c3ccc(-c4cn(-c5cccc(S(=O)(=O)O)c5)nn4)o3)nn2)cc1C(F)(F)F] |
| 317 | m-(4-{5-[1-(m-Sulfophenyl)-1H-1,2,3- triazol-4-yl]-2-furyl}-1H-1,2,3-triazol-1- yl)benzenesulfonic acid | 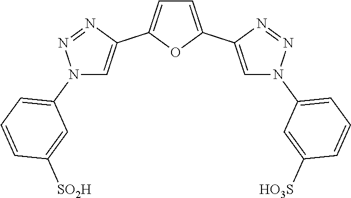 |
| 318 | 0-(4-{5-[1-(o-Sulfophenyl)-1H-1,2,3-triazol- 4-yl]-2-furyl}-1H-1,2,3-triazol-1- yl)benzenesulfonic acid | 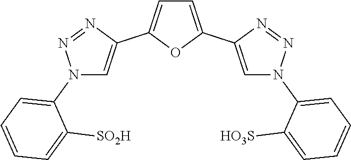 |
| 319 | 4-(4-{5-[1-(4-Carboxy-3-chlorophenyl)-1H- 1,2,3-triazol-4-yl]-2-furyl}-1H-1,2,3-triazol- 1-yl)-2-chlorobenzoic acid | 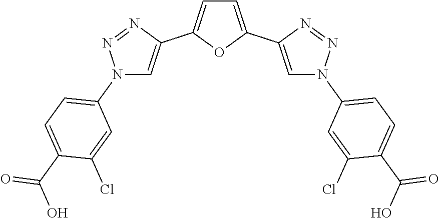 [SMILES: O=C(O)c1ccc(-n2cc(-c3ccc(-c4cn(-c5ccc(C(=O)O)c(Cl)c5)nn4)o3)nn2)cc1Cl] |
| 320 | 4-[4-(7-{1-[4-Carboxy-3- (trifluoromethyl)phenyl]-1H-1,2,3-triazol-4- yl}-2-carbazolyl)-1H-1,2,3-triazol-1-yl]-2- (trifluoromethyl)benzoic acid | 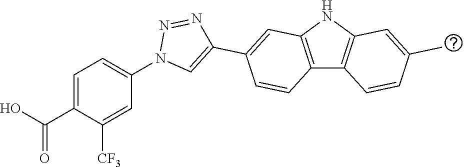 [SMILES: Cc1ccc2c(c1)[nH]c1cc(-c3cn(-c4ccc(C(=O)O)c(C(F)(F)F)c4)nn3)ccc12] |
| 321 | 4-[4-(7-{1-[4-Carboxy-3- (trifluoromethyl)phenyl]-1H-1,2,3-triazol-4- yl}-9-oxa-2-fluorenyl)-1H-1,2,3-triazol-1- yl]-2-(trifluoromethyl)benzoic acid | 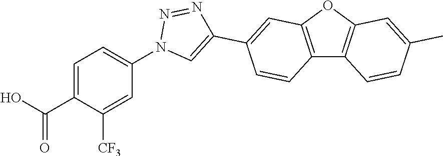 [SMILES: Cc1ccc2c(c1)oc1cc(-c3cn(-c4ccc(C(=O)O)c(C(F)(F)F)c4)nn3)ccc12] |
| 322 | 5-(4-{5-[1-(4-Sulfo-3-tolyl)-1H-1,2,3-triazol- 4-yl]-2-furyl}-1H-1,2,3-triazol-1-yl)-2- toluenesulfonic acid | 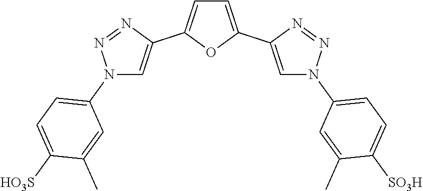 [SMILES: Cc1cc(-n2cc(-c3ccc(-c4cn(-c5ccc(S(=O)(=O)O)c(C)c5)nn4)o3)nn2)ccc1S(=O)(=O)O] |
| 323 | 4-(4-{5-[1-(2,4-Disulfophenyl)-1H-1,2,3- triazol-4-yl]-2-furyl}-1H-1,2,3-triazol-1-yl)- 1,3-benzenedisulfonic acid | 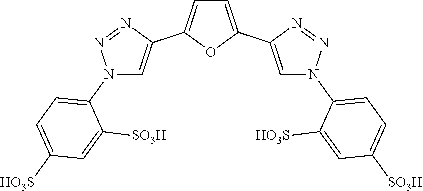 [SMILES: O=S(=O)(O)c1ccc(-n2cc(-c3ccc(-c4cn(-c5ccc(S(=O)(=O)O)cc5S(=O)(=O)O)nn4)o3)nn2)c(S(=O)(=O)O)c1] |
| 324 | 4-(4-{5-[1-(4-Sulfo-3-tolyl)-1H-1,2,3-triazol- 4-yl]-2-furyl}-1H-1,2,3-triazol-1-yl)-2- (trifluoromethyl)benzoic acid | 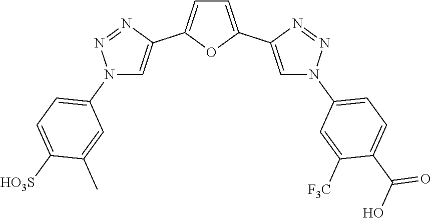 [SMILES: Cc1cc(-n2cc(-c3ccc(-c4cn(-c5ccc(C(=O)O)c(C(F)(F)F)c5)nn4)o3)nn2)ccc1S(=O)(=O)O] |
| 325 | 3-[2-(Trifluoromethyl)-4-{4-[4-(N- ethylcarbamoyl)-6-{1-[4-(N-5-carboxy-1H- 1,2,4-triazol-3-ylcarbamoyl)-3- (trifluoromethyl)phenyl]-1H-1,2,3-triazol-4- yl}-2-pyridyl]-1H-1,2,3-triazol-1- yl}benzylamino]-1H-1,2,4-triazole-5- carboxylic acid | 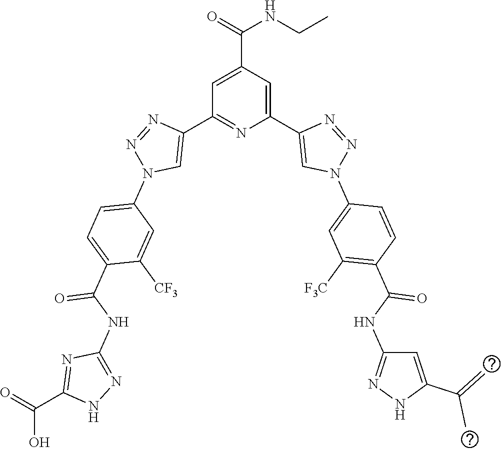 [SMILES: C=C(C)c1cc(NC(=O)c2ccc(-n3cc(-c4cc(C(=O)NCC)cc(-c5cn(-c6ccc(C(=O)Nc7n[nH]c(C(=O)O)n7)c(C(F)(F)F)c6)nn5)n4)nn3)cc2C(F)(F)F)n[nH]1] |
| 326 | 4-[4-(6-{1-[4-Carboxy-3- (trifluoromethyl)phenyl]-1H-1,2,3-triazol-4- yl}-3-carbazolyl)-1H-1,2,3-triazol-1-yl]-2- (trifluoromethyl)benzoic acid | 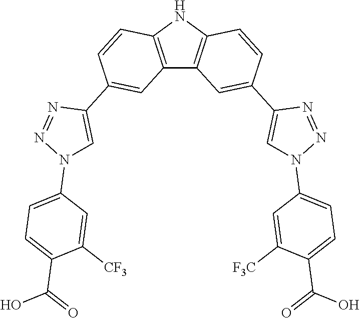 [SMILES: O=C(O)c1ccc(-n2cc(-c3ccc4[nH]c5ccc(-c6cn(-c7ccc(C(=O)O)c(C(F)(F)F)c7)nn6)cc5c4c3)nn2)cc1C(F)(F)F] |
| 327 | 4-[4-(5-{1-[4-Carboxy-3- (trifluoromethyl)phenyl]-1H-1,2,3-triazol-4- yl}-2-selenophenyl)-1H-1,2,3-triazol-1-yl]-2- (trifluoromethyl)benzoic acid | 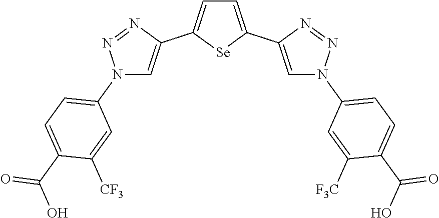 [SMILES: O=C(O)c1ccc(-n2cc(-c3ccc(-c4cn(-c5ccc(C(=O)O)c(C(F)(F)F)c5)nn4)[se]3)nn2)cc1C(F)(F)F] |
| 328 | 4-[4-(6-{1-[4-Carboxy-3- (trifluoromethyl)phenyl]-1H-1,2,3-triazol-4- yl}-3-carbazolyl)-1H-1,2,3-triazol-1-yl]-2- (trifluoromethyl)benzoic acid | 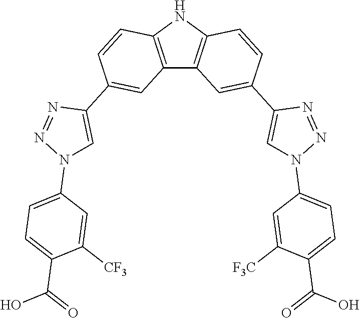 [SMILES: O=C(O)c1ccc(-n2cc(-c3ccc4[nH]c5ccc(-c6cn(-c7ccc(C(=O)O)c(C(F)(F)F)c7)nn6)cc5c4c3)nn2)cc1C(F)(F)F] |
| 329 | 4-[4-(9-Acetyl-6-{1-[4-carboxy-3- (trifluoromethyl)phenyl]-1H-1,2,3-triazol-4- yl}-3-carbazolyl)-1H-1,2,3-triazol-1-yl]-2- (trifluoromethyl)benzoic acid | 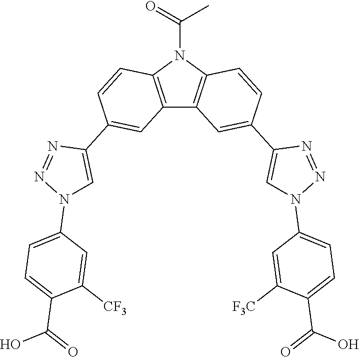 [SMILES: CC(=O)n1c2ccc(-c3cn(-c4ccc(C(=O)O)c(C(F)(F)F)c4)nn3)cc2c2cc(-c3cn(-c4ccc(C(=O)O)c(C(F)(F)F)c4)nn3)ccc21] |
| 330 | 4-[4-(8-{1-[4-Carboxy-3- (trifluoromethyl)phenyl]-1H-1,2,3-triazol-4- yl}-1-carbazolyl)-1H-1,2,3-triazol-1-yl]-2- (trifluoromethyl)benzoic acid | 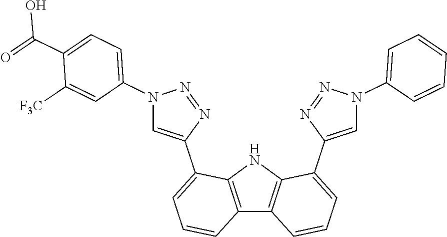 [SMILES: O=C(O)c1ccc(-n2cc(-c3cccc4c3[nH]c3c(-c5cn(-c6ccccc6)nn5)cccc34)nn2)cc1C(F)(F)F] |
| 331 | 4-[4-(7-{1-[4-Carboxy-3- (trifluoromethyl)phenyl]-1H-1,2,3-triazol-4- yl}-1,8-diaza-2-naphthyl)-1H-1,2,3-triazol-1- yl]-2-(trifluoromethyl)benzoic acid | 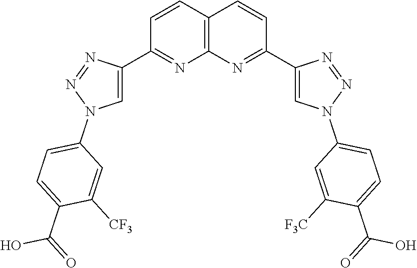 [SMILES: O=C(O)c1ccc(-n2cc(-c3ccc4ccc(-c5cn(-c6ccc(C(=O)O)c(C(F)(F)F)c6)nn5)nc4n3)nn2)cc1C(F)(F)F] |
| 332 | 4-[4-(6-{1-[4-Carboxy-3- (trifluoromethyl)phenyl]-1H-1,2,3-triazol-4- yl}-9-hydroxy-3-carbazolyl)-1H-1,2,3- triazol-1-yl]-2-(trifluoromethyl)benzoic acid | 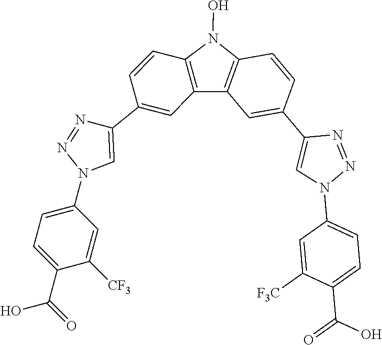 [SMILES: O=C(O)c1ccc(-n2cc(-c3ccc4c(c3)c3cc(-c5cn(-c6ccc(C(=O)O)c(C(F)(F)F)c6)nn5)ccc3n4O)nn2)cc1C(F)(F)F] |
| 333 | 4-[4-(7-{1-[4-Carboxy-3- (trifluoromethyl)phenyl]-1H-1,2,3-triazol-4- yl}-2-naphthyl)-1H-1,2,3-triazol-1-yl]-2- (trifluoromethyl)benzoic acid | 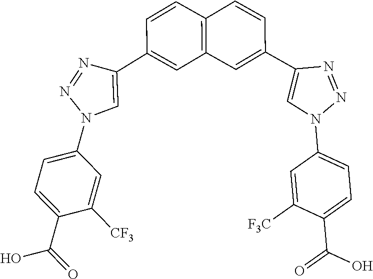 [SMILES: O=C(O)c1ccc(-n2cc(-c3ccc4ccc(-c5cn(-c6ccc(C(=O)O)c(C(F)(F)F)c6)nn5)cc4c3)nn2)cc1C(F)(F)F] |
| 334 | 4-[4-(6-{1-[4-Carboxy-3- (trifluoromethyl)phenyl]-1H-1,2,3-triazol-4- yl}-4,5-diaza-3-phenanthryl)-1H-1,2,3- triazol-1-yl]-2-(trifluoromethyl)benzoic acid | 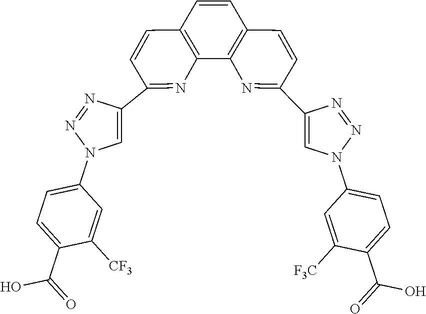 [SMILES: O=C(O)c1ccc(-n2cc(-c3ccc4ccc5ccc(-c6cn(-c7ccc(C(=O)O)c(C(F)(F)F)c7)nn6)nc5c4n3)nn2)cc1C(F)(F)F] |
| 335 | 4-[4-(6-{1-[4-Carboxy-3- (trifluoromethyl)phenyl]-1H-1,2,3-triazol-4- yl}-9-(3-hydroxypropionyl)-3-carbazolyl)- 1H-1,2,3-triazol-1-yl]-2- (trifluoromethyl)benzoic acid | 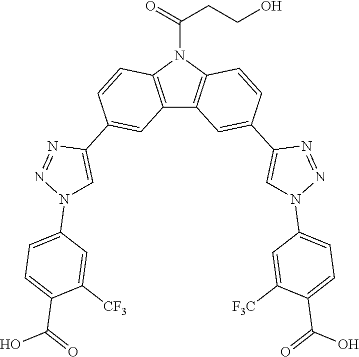 [SMILES: O=C(O)c1ccc(-n2cc(-c3ccc4c(c3)c3cc(-c5cn(-c6ccc(C(=O)O)c(C(F)(F)F)c6)nn5)ccc3n4C(=O)CCO)nn2)cc1C(F)(F)F] |
| 336 | 4-[4-(6-{1-[4-Carboxy-3- (trifluoromethyl)phenyl]-1H-1,2,3-triazol-4- yl}-9-[3-(3-hydroxypropionoxy)propionyl]- 3-carbazolyl)-1H-1,2,3-triazol-1-yl]-2- (trifluoromethyl)benzoic acid | 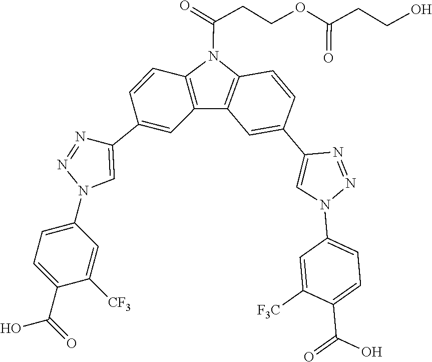 [SMILES: O=C(CCO)OCCC(=O)n1c2ccc(-c3cn(-c4ccc(C(=O)O)c(C(F)(F)F)c4)nn3)cc2c2cc(-c3cn(-c4ccc(C(=O)O)c(C(F)(F)F)c4)nn3)ccc21] |
| 337 | 2-(Trifluoromethyl)-4-[4-(6-{1-[4-(N,N- dimethylcarbamoyl)-3- (trifluoromethyl)phenyl]-1H-1,2,3-triazol-4- yl}-3-carbazolyl)-1H-1,2,3-triazol-1- yl]benzoic acid | 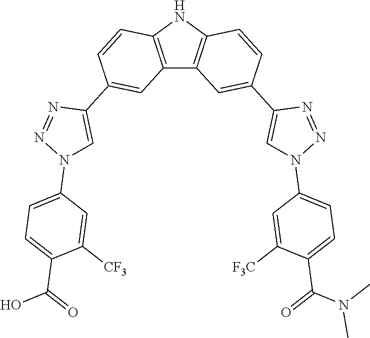 [SMILES: CN(C)C(=O)c1ccc(-n2cc(-c3ccc4[nH]c5ccc(-c6cn(-c7ccc(C(=O)O)c(C(F)(F)F)c7)nn6)cc5c4c3)nn2)cc1C(F)(F)F] |
| 338 | p-{4-[4-(Allylaminosulfonyl)-6-[1-(p- sulfophenyl)-1H-1,2,3-triazol-4-yl]-2- pyridyl]-1H-1,2,3-triazol-1- yl} benzenesulfonic acid | 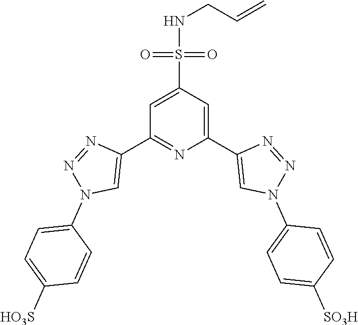 [SMILES: C=CCNS(=O)(=O)c1cc(-c2cn(-c3ccc(S(=O)(=O)O)cc3)nn2)nc(-c2cn(-c3ccc(S(=O)(=O)O)cc3)nn2)c1] |
| 339 | p-{4-[4-(2-Hydroxyethylaminosulfonyl)-6- [1-(p-sulfophenyl)-1H-1,2,3-triazol-4-yl]-2- pyridyl]-1H-1,2,3-triazol-1- yl} benzenesulfonic acid | 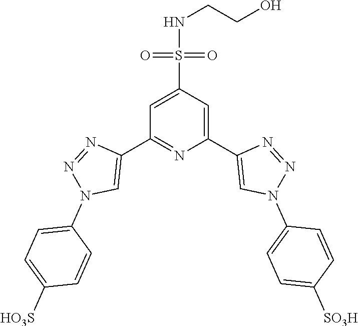 [SMILES: O=S(=O)(O)c1ccc(-n2cc(-c3cc(S(=O)(=O)NCCO)cc(-c4cn(-c5ccc(S(=O)(=O)O)cc5)nn4)n3)nn2)cc1] |
| 340 | p-{4-[4-(Isopropylaminosulfonyl)-6-[1-(p- sulfophenyl)-1H-1,2,3-triazol-4-yl]-2- pyridyl]-1H-1,2,3-triazol-1- yl} benzenesulfonic acid | 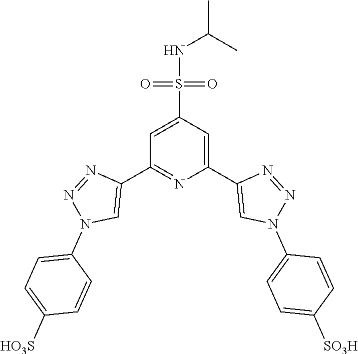 [SMILES: CC(C)NS(=O)(=O)c1cc(-c2cn(-c3ccc(S(=O)(=O)O)cc3)nn2)nc(-c2cn(-c3ccc(S(=O)(=O)O)cc3)nn2)c1] |
| 341 | p-{4-[4-(Morpholinosulfonyl)-6-[1-(p- sulfophenyl)-1H-1,2,3-triazol-4-yl]-2- pyridyl]-1H-1,2,3-triazol-1- yl }benzenesulfonic acid | 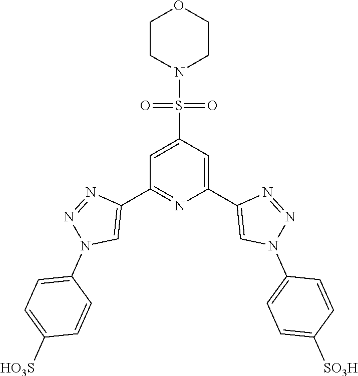 [SMILES: O=S(=O)(O)c1ccc(-n2cc(-c3cc(S(=O)(=O)N4CCOCC4)cc(-c4cn(-c5ccc(S(=O)(=O)O)cc5)nn4)n3)nn2)cc1] |
| 342 | p-[4-(4-{[(2,2-Dimethyl-1,3-dioxolan-4- yl)methyl]aminosulfonyl}-6-[1-(p- sulfophenyl)-1H-1,2,3-triazol-4-yl]-2- pyridyl)-1H-1,2,3-triazol-1- yl]benzenesulfonic acid | 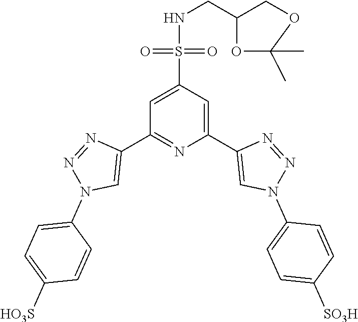 [SMILES: CC1(C)OCC(CNS(=O)(=O)c2cc(-c3cn(-c4ccc(S(=O)(=O)O)cc4)nn3)nc(-c3cn(-c4ccc(S(=O)(=O)O)cc4)nn3)c2)O1] |
| 343 | p-{4-[4-(1-Piperazinylsulfonyl)-6-[1-(p- sulfophenyl)-1H-1,2,3-triazol-4-yl]-2- pyridyl]-1H-1,2,3-triazol-1- yl}benzenesulfonic acid | 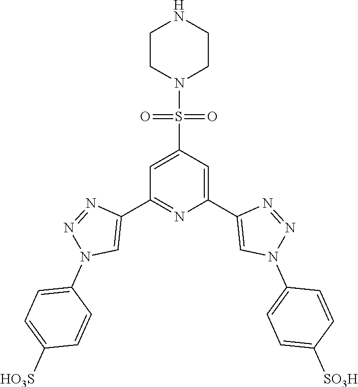 [SMILES: O=S(=O)(O)c1ccc(-n2cc(-c3cc(S(=O)(=O)N4CCNCC4)cc(-c4cn(-c5ccc(S(=O)(=O)O)cc5)nn4)n3)nn2)cc1] |
| 344 | p-{4-[4-(4-tert-Butoxycarbonyl-1- piperazinylsulfonyl)-6-[1-(p-sulfophenyl)- 1H-1,2,3-triazol-4-yl]-2-pyridyl]-1H-1,2,3- triazol-1-yl}benzenesulfonic acid | 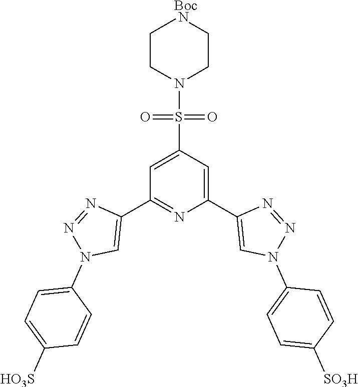 |
| 345 | p-(4-{4-Aminosulfonyl-6-[1-(p-sulfophenyl)- 1H-1,2,3-triazol-4-yl]-2-pyridyl}-1H-1,2,3- triazol-1-yl)benzenesulfonic acid | 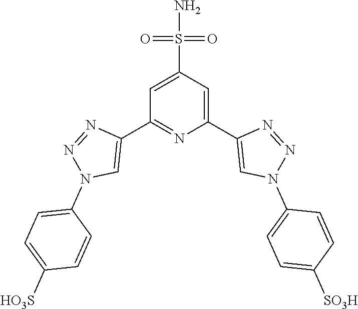 [SMILES: NS(=O)(=O)c1cc(-c2cn(-c3ccc(S(=O)(=O)O)cc3)nn2)nc(-c2cn(-c3ccc(S(=O)(=O)O)cc3)nn2)c1] |
| 346 | p-{4-[4-(2,3- Dihydroxypropylaminosulfonyl)-6-[1-(p- sulfophenyl)-1H-1,2,3-triazol-4-yl]-2- pyridyl]-1H-1,2,3-triazol-1- yl}benzenesulfonic acid | 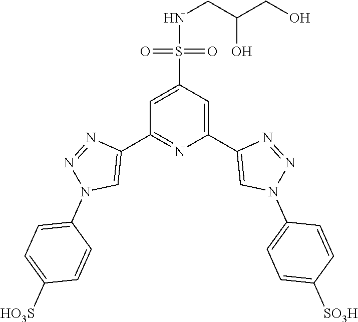 [SMILES: O=S(=O)(O)c1ccc(-n2cc(-c3cc(S(=O)(=O)NCC(O)CO)cc(-c4cn(-c5ccc(S(=O)(=O)O)cc5)nn4)n3)nn2)cc1] |
| 347 | p-{4-[4-(2,2-Dimethyl-1,3-dioxan-5- ylaminosulfonyl)-6-[1-(p-sulfophenyl)-1H- 1,2,3-triazol-4-yl]-2-pyridyl]-1H-1,2,3- triazol-1-yl}benzenesulfonic acid | 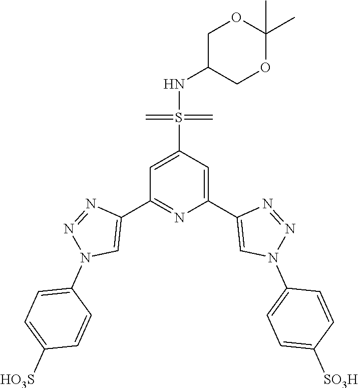 [SMILES: C=S(=C)(NC1COC(C)(C)OC1)c1cc(-c2cn(-c3ccc(S(=O)(=O)O)cc3)nn2)nc(-c2cn(-c3ccc(S(=O)(=O)O)cc3)nn2)c1] |
| 348 | p-(4-{4-[2-Hydroxy-1- (hydroxymethyl)ethylaminosulfonyl]-6-[1- (p-sulfophenyl)-1H-1,2,3-triazol-4-yl]-2- pyridyl}-1H-1,2,3-triazol-1- yl)benzenesulfonic acid | 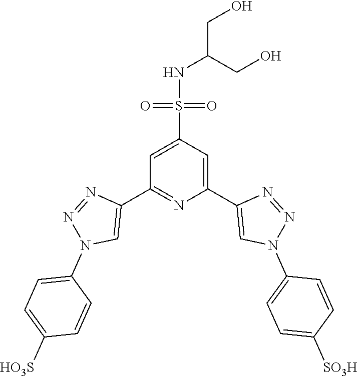 [SMILES: O=S(=O)(O)c1ccc(-n2cc(-c3cc(S(=O)(=O)NC(CO)CO)cc(-c4cn(-c5ccc(S(=O)(=O)O)cc5)nn4)n3)nn2)cc1] |
| 349 | p-(4-{4-[(tert- Butoxycarbonylmethyl)aminosulfonyl]-6-[1- (p-sulfophenyl)-1H-1,2,3-triazol-4-yl]-2- pyridyl}-1H-1,2,3-triazol-1- yl)benzenesulfonic acid | 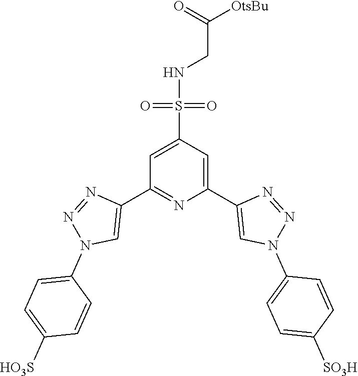 |
| 350 | 2,6-Bis[1-(p-sulfophenyl)-1H-1,2,3-triazol-4- yl]-4-pyridinesulfonic acid | 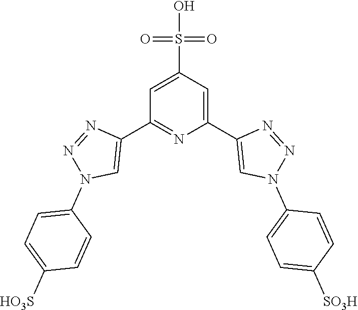 [SMILES: O=S(=O)(O)c1ccc(-n2cc(-c3cc(S(=O)(=O)O)cc(-c4cn(-c5ccc(S(=O)(=O)O)cc5)nn4)n3)nn2)cc1] |
| 351 | p-(4-{4-Methoxycarbonyl-6-[1-(p- sulfophenyl)-1H-1,2,3-triazol-4-yl]-2- pyridyl}-1H-1,2,3-triazol-1- yl)benzenesulfonic acid | 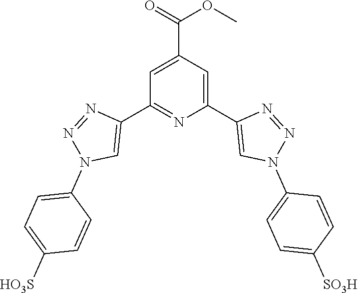 [SMILES: COC(=O)c1cc(-c2cn(-c3ccc(S(=O)(=O)O)cc3)nn2)nc(-c2cn(-c3ccc(S(=O)(=O)O)cc3)nn2)c1] |
| 352 | p-(4-{4-[N-2-Hydroxy-1- (hydroxymethyl)ethylcarbamoyl]-6-[1-(p- sulfophenyl)-1H-1,2,3-triazol-4-yl]-2- pyridyl}-1H-1,2,3-triazol-1- yl)benzenesulfonic acid | 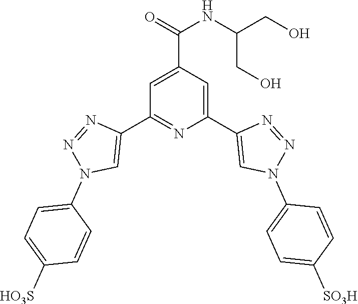 [SMILES: O=C(NC(CO)CO)c1cc(-c2cn(-c3ccc(S(=O)(=O)O)cc3)nn2)nc(-c2cn(-c3ccc(S(=O)(=O)O)cc3)nn2)c1] |
| 353 | p-{4-[4-(N-Butylcarbamoyl)-6-[1-(p- sulfophenyl)-1H-1,2,3-triazol-4-yl]-2- pyridyl]-1H-1,2,3-triazol-1- yl}benzenesulfonic acid | 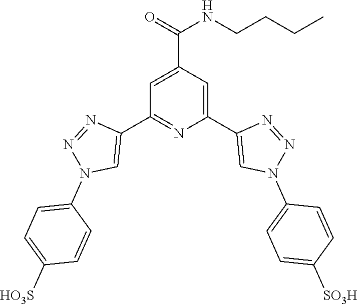 [SMILES: CCCCNC(=O)c1cc(-c2cn(-c3ccc(S(=O)(=O)O)cc3)nn2)nc(-c2cn(-c3ccc(S(=O)(=O)O)cc3)nn2)c1] |
| 354 | p-{4-[4-(N-Allylcarbamoyl)-6-[1-(p- sulfophenyl)-1H-1,2,3-triazol-4-yl]-2- pyridyl]-1H-1,2,3-triazol-1- yl}benzenesulfonic acid | 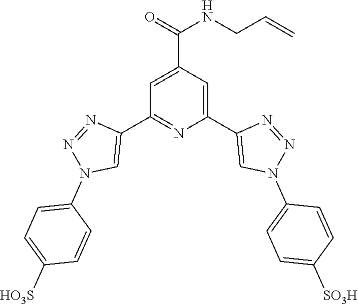 [SMILES: C=CCNC(=O)c1cc(-c2cn(-c3ccc(S(=O)(=O)O)cc3)nn2)nc(-c2cn(-c3ccc(S(=O)(=O)O)cc3)nn2)c1] |
| 355 | p-[4-({[1-(p-Sulfophenyl)-1H-1,2,3-triazol-4- yl]methoxy}methyl)-1H-1,2,3-triazol-1- yl]benzenesulfonic acid | 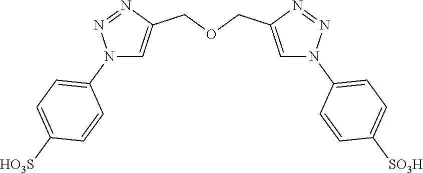 [SMILES: O=S(=O)(O)c1ccc(-n2cc(COCc3cn(-c4ccc(S(=O)(=O)O)cc4)nn3)nn2)cc1] |
| 356 | 5-(4-{6-[1-(3-Carboxy-5-pyrazolyl)-1H- 1,2,3-triazol-4-yl]-4-(ethylaminosulfonyl)-2- pyridyl}-1H-1,2,3-triazol-1-yl)-3- pyrazolecarboxylic acid | 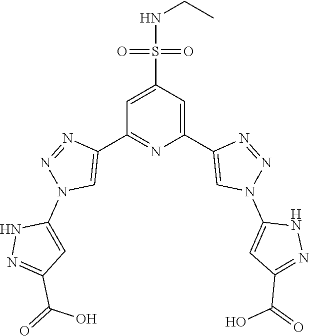 [SMILES: CCNS(=O)(=O)c1cc(-c2cn(-c3cc(C(=O)O)n[nH]3)nn2)nc(-c2cn(-c3cc(C(=O)O)n[nH]3)nn2)c1] |
| 357 | p-(4-{4-Carbamoyl-6-[1-(p-sulfophenyl)-1H- 1,2,3-triazol-4-yl]-2-pyridyl}-1H-1,2,3- triazol-1-yl)benzenesulfonic acid | 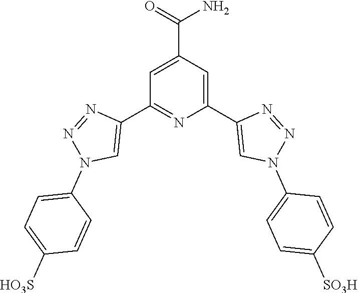 [SMILES: NC(=O)c1cc(-c2cn(-c3ccc(S(=O)(=O)O)cc3)nn2)nc(-c2cn(-c3ccc(S(=O)(=O)O)cc3)nn2)c1] |
| 358 | p-(4-{4-Cyano-6-[1-(p-sulfophenyl)-1H- 1,2,3-triazol-4-yl]-2-pyridyl}-1H-1,2,3- triazol-1-yl)benzenesulfonic acid | 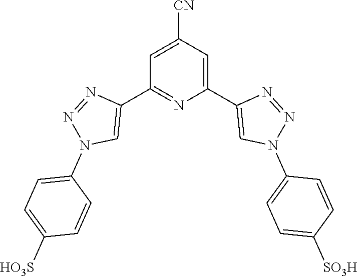 [SMILES: N#Cc1cc(-c2cn(-c3ccc(S(=O)(=O)O)cc3)nn2)nc(-c2cn(-c3ccc(S(=O)(=O)O)cc3)nn2)c1] |
| 359 | p-(4-{4-Ethoxycarbonyl-6-[1-(p- sulfophenyl)-1H-1,2,3-triazol-4-yl]-2- pyridyl}-1H-1,2,3-triazol-1- yl)benzenesulfonic acid | 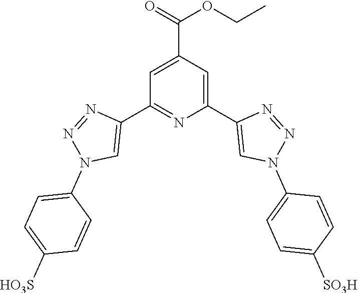 [SMILES: CCOC(=O)c1cc(-c2cn(-c3ccc(S(=O)(=O)O)cc3)nn2)nc(-c2cn(-c3ccc(S(=O)(=O)O)cc3)nn2)c1] |
| 360 | p-{4-[4-(N-Acetylcarbamoyl)-6-[1-(p- sulfophenyl)-1H-1,2,3-triazol-4-yl]-2- pyridyl]-1H-1,2,3-triazol-1- yl}benzenesulfonic acid | 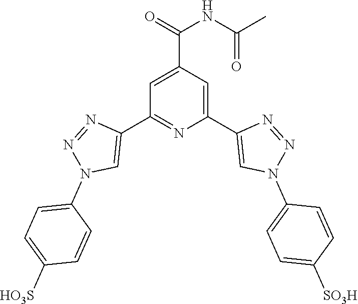 [SMILES: CC(=O)NC(=O)c1cc(-c2cn(-c3ccc(S(=O)(=O)O)cc3)nn2)nc(-c2cn(-c3ccc(S(=O)(=O)O)cc3)nn2)c1] |
| 361 | 2-[(E)-2-Methoxyethenyl]-6-[1-(p- sulfophenyl)-1H-1,2,3-triazol-4-yl]-4- pyridinesulfonic acid | 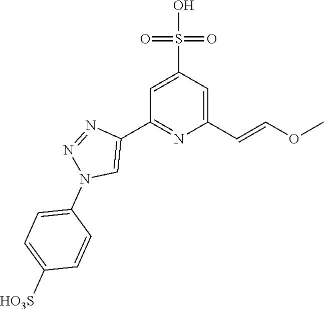 [SMILES: CO/C=C/c1cc(S(=O)(=O)O)cc(-c2cn(-c3ccc(S(=O)(=O)O)cc3)nn2)n1] |
| 362 | 5-(4-{6-[1-(4-Carboxy-5-pyrazolyl)-1H- 1,2,3-triazol-4-yl]-4-(ethylaminosulfonyl)-2- pyridyl}-1H-1,2,3-triazol-1-yl)-4- pyrazolecarboxylic acid | 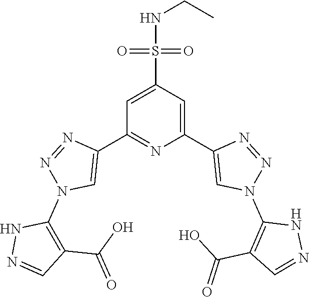 [SMILES: CCNS(=O)(=O)c1cc(-c2cn(-c3[nH]ncc3C(=O)O)nn2)nc(-c2cn(-c3[nH]ncc3C(=O)O)nn2)c1] |
| 363 | p-{4-[4-(N-Benzylcarbamoyl)-6-[1-(p- sulfophenyl)-1H-1,2,3-triazol-4-yl]-2- pyridyl]-1H-1,2,3-triazol-1- yl}benzenesulfonic acid | 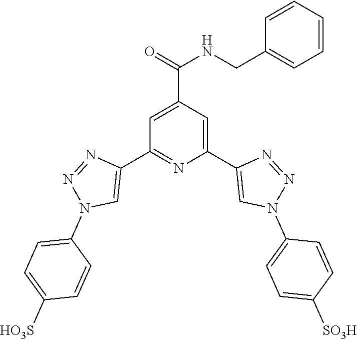 [SMILES: O=C(NCc1ccccc1)c1cc(-c2cn(-c3ccc(S(=O)(=O)O)cc3)nn2)nc(-c2cn(-c3ccc(S(=O)(=O)O)cc3)nn2)c1] |
| 364 | p-{4-[4-(N-2-Propynylcarbamoyl)-6-[1-(p- sulfophenyl)-1H-1,2,3-triazol-4-yl]-2- pyridyl]-1H-1,2,3-triazol-1- yl}benzenesulfonic acid | 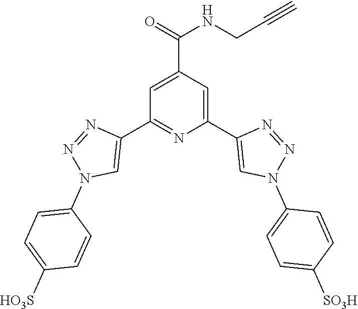 [SMILES: C#CCNC(=O)c1cc(-c2cn(-c3ccc(S(=O)(=O)O)cc3)nn2)nc(-c2cn(-c3ccc(S(=O)(=O)O)cc3)nn2)c1] |
| 365 | p-{4-[4-(N-Propylcarbamoyl)-6-[1-(p- sulfophenyl)-1H-1,2,3-triazol-4-yl]-2- pyridyl]-1H-1,2,3-triazol-1- yl}benzenesulfonic acid | 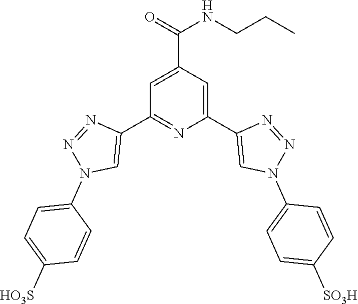 [SMILES: CCCNC(=O)c1cc(-c2cn(-c3ccc(S(=O)(=O)O)cc3)nn2)nc(-c2cn(-c3ccc(S(=O)(=O)O)cc3)nn2)c1] |
| 366 | {2,6-Bis[1-(p-sulfophenyl)-1H-1,2,3-triazol- 4-yl]-4-pyridylsulfonylamino}acetic acid | 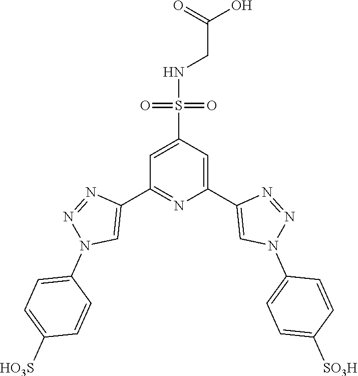 [SMILES: O=C(O)CNS(=O)(=O)c1cc(-c2cn(-c3ccc(S(=O)(=O)O)cc3)nn2)nc(-c2cn(-c3ccc(S(=O)(=O)O)cc3)nn2)c1] |
| 367 | p-{1-[4-(Ethylaminosulfonyl)-6-[4-(p- sulfophenyl)-1H-1,2,3-triazol-1-yl]-2- pyridyl]-1H-1,2,3-triazol-4- yl}benzenesulfonic acid | 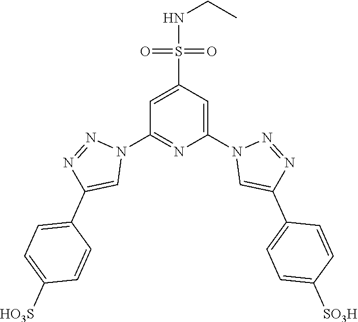 [SMILES: CCNS(=O)(=O)c1cc(-n2cc(-c3ccc(S(=O)(=O)O)cc3)nn2)nc(-n2cc(-c3ccc(S(=O)(=O)O)cc3)nn2)c1] |
| 368 | (S)-1-[4-(4-{5-[1-(4-{[(S)-2-Carboxy-1- pyrrolidinyl]carbonyl}-3- (trifluoromethyl)phenyl)-1H-1,2,3-triazol-4- yl]-2-furyl}-1H-1,2,3-triazol-1-yl)-2- (trifluoromethyl)benzoyl]-2- pyrrolidinecarboxylic acid | 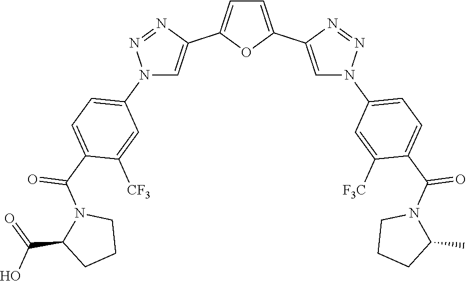 [SMILES: C[C@@H]1CCCN1C(=O)c1ccc(-n2cc(-c3ccc(-c4cn(-c5ccc(C(=O)N6CCC[C@H]6C(=O)O)c(C(F)(F)F)c5)nn4)o3)nn2)cc1C(F)(F)F] |
| 369 | (2S,4R)-1-[4-(4-{5-[1-(4-{[(2S,4R)-2- Carboxy-4-hydroxy-1- pyrrolidinyl]carbonyl}-3- (trifluoromethyl)phenyl)-1H-1,2,3-triazol-4- yl]-2-furyl}-1H-1,2,3-triazol-1-yl)-2- (trifluoromethyl)benzoyl]-4-hydroxy-2- pyrrolidinecarboxylic acid | 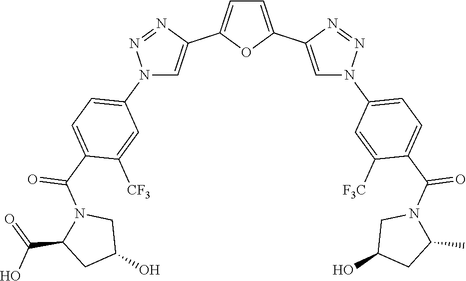 [SMILES: C[C@@H]1C[C@@H](O)CN1C(=O)c1ccc(-n2cc(-c3ccc(-c4cn(-c5ccc(C(=O)N6C[C@H](O)C[C@H]6C(=O)O)c(C(F)(F)F)c5)nn4)o3)nn2)cc1C(F)(F)F] |
| 370 | 1-(4-{4-[5-(1-{4-[(3-Carboxy-1- azetidinyl)carbonyl]-3- (trifluoromethyl)phenyl}-1H-1,2,3-triazol-4- yl)-2-furyl]-1H-1,2,3-triazol-1-yl}-2- (trifluoromethyl)benzoyl)-3- azetidinecarboxylic acid | 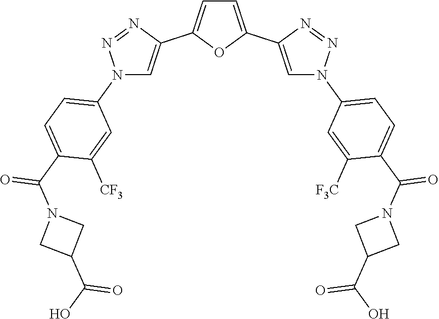 [SMILES: O=C(O)C1CN(C(=O)c2ccc(-n3cc(-c4ccc(-c5cn(-c6ccc(C(=O)N7CC(C(=O)O)C7)c(C(F)(F)F)c6)nn5)o4)nn3)cc2C(F)(F)F)C1] |
| 371 | p-{4-[4-(3-Hydroxypropylaminosulfonyl)-6- [1-(p-sulfophenyl)-1H-1,2,3-triazol-4-yl]-2- pyridyl]-1H-1,2,3-triazol-1- yl}benzenesulfonic acid | 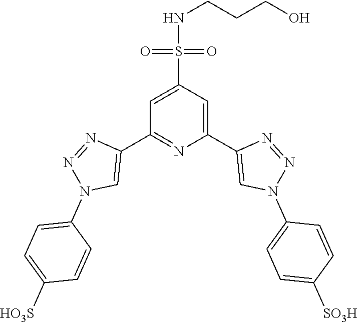 [SMILES: O=S(=O)(O)c1ccc(-n2cc(-c3cc(S(=O)(=O)NCCCO)cc(-c4cn(-c5ccc(S(=O)(=O)O)cc5)nn4)n3)nn2)cc1] |
| 372 | p-{4-[4-(Methylaminosulfonyl)-6-[1-(p- sulfophenyl)-1H-1,2,3-triazol-4-yl]-2- pyridyl]-1H-1,2,3-triazol-1- yl}benzenesulfonic acid | 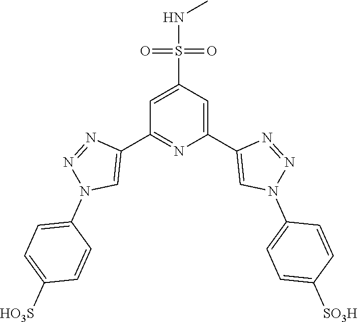 [SMILES: CNS(=O)(=O)c1cc(-c2cn(-c3ccc(S(=O)(=O)O)cc3)nn2)nc(-c2cn(-c3ccc(S(=O)(=O)O)cc3)nn2)c1] |
| 373 | p-(4-{4-[(Methoxyamino)sulfonyl]-6-[1-(p- sulfophenyl)-1H-1,2,3-triazol-4-yl]-2- pyridyl}-1H-1,2,3-triazol-1- yl)benzenesulfonic acid | 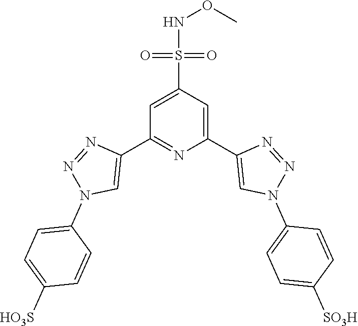 [SMILES: CONS(=O)(=O)c1cc(-c2cn(-c3ccc(S(=O)(=O)O)cc3)nn2)nc(-c2cn(-c3ccc(S(=O)(=O)O)cc3)nn2)c1] |
| 374 | p-(4-{4-[1-(2-Hydroxyethyl)-1H-1,2,3- triazol-4-yl]-6-[1-(p-sulfophenyl)-1H-1,2,3- triazol-4-yl]-2-pyridyl}-1H-1,2,3-triazol-1- yl)benzenesulfonic acid | 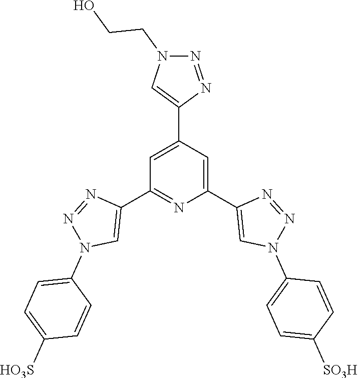 [SMILES: O=S(=O)(O)c1ccc(-n2cc(-c3cc(-c4cn(CCO)nn4)cc(-c4cn(-c5ccc(S(=O)(=O)O)cc5)nn4)n3)nn2)cc1] |
| 375 | p-{4-[4-(Hydrazinosulfonyl)-6-[1-(p- sulfophenyl)-1H-1,2,3-triazol-4-yl]-2- pyridyl]-1H-1,2,3-triazol-1- yl} benzenesulfonic acid | 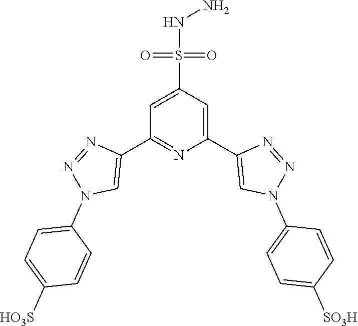 [SMILES: NNS(=O)(=O)c1cc(-c2cn(-c3ccc(S(=O)(=O)O)cc3)nn2)nc(-c2cn(-c3ccc(S(=O)(=O)O)cc3)nn2)c1] |
| 376 | p-(4-{6-[1-(p-Sulfophenyl)-1H-1,2,3-triazol- 4-yl]-2-pyridyl}-1H-1,2,3-triazol-1- yl)benzenesulfonic acid264 | 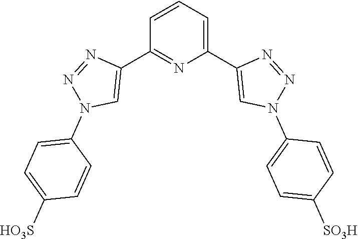 [SMILES: O=S(=O)(O)c1ccc(-n2cc(-c3cccc(-c4cn(-c5ccc(S(=O)(=O)O)cc5)nn4)n3)nn2)cc1] |
| 377 | p-(4-{6-[1-(p-Sulfophenyl)-1H-1,2,3-triazol- 4-yl]-4-(trifluoromethyl)-2-pyridyl}-1H- 1,2,3-triazol-1-yl)benzenesulfonic acid264 | 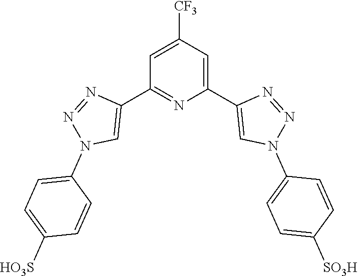 [SMILES: O=S(=O)(O)c1ccc(-n2cc(-c3cc(C(F)(F)F)cc(-c4cn(-c5ccc(S(=O)(=O)O)cc5)nn4)n3)nn2)cc1] |
| 378 | p-(4-{4-Methoxy-6-[1-(p-sulfophenyl)-1H- 1,2,3-triazol-4-yl]-2-pyridyl}-1H-1,2,3- triazol-1-yl)benzenesulfonic acid265 | 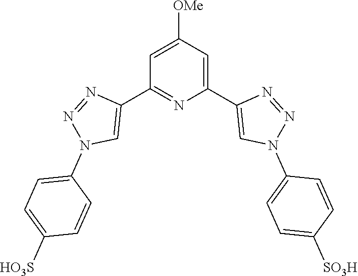 [SMILES: COc1cc(-c2cn(-c3ccc(S(=O)(=O)O)cc3)nn2)nc(-c2cn(-c3ccc(S(=O)(=O)O)cc3)nn2)c1] |
| 379 | | 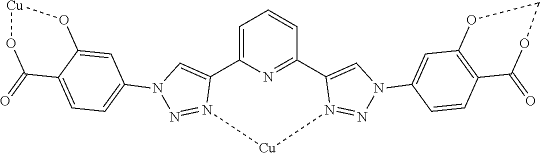 |
| 380 | | 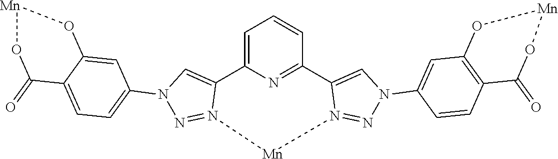 |
| 381 | | 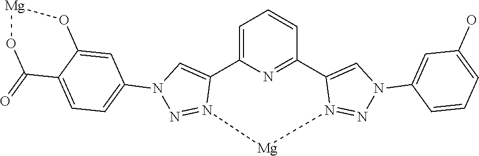 |
| 382 | | 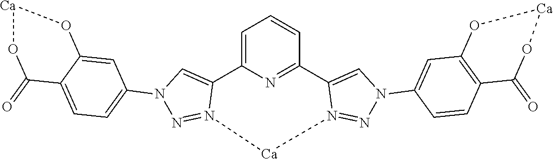 |

[symbol]

indicates data missing or illegible when filed

While the compounds set forth in Table 1 include a triazole “Z” group (in terms of Formula (I)), other heteroaromatic groups may be substituted for the triazole, where the other heteroaromatic groups may include one, two, three, or four heteroatoms, such as one, two, three, or four heteroatoms selected from O, N, and/or S. By way of example, each triazole may be substituted with an imidazole, a pyrazole, a pyrimidine, a pyridazine, a pyrazine, a pyrrole, a thiophene, a thiazole, an isoxazole, or a tetrazole.

In some embodiments, the compounds set forth in Table 1 may be polymerized (e.g., via cationic polymerization, such as by incubating a PEM with KCl). In some embodiments, a composition may include two or more molecules of the compounds set forth in Table 1.

### Example 1

### Synthesis of 4,4′-(pyridine-2,6-diylbis(1H-1,2,3-triazole-4,1-diyl))bis(2-hydroxybenzoic acid)

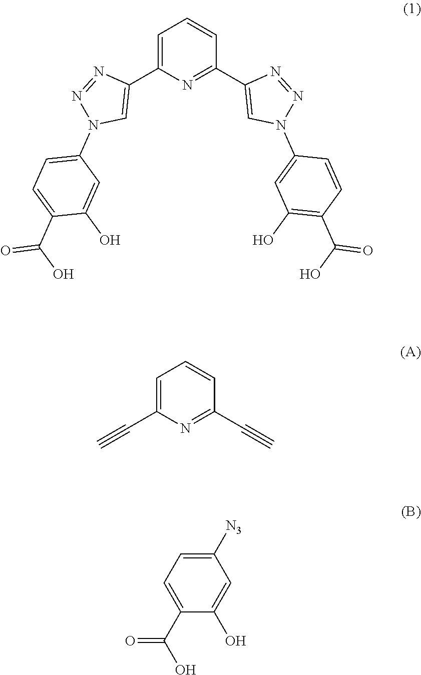

Compound 1 was prepared by mixing 4-azidosalicylic acid B (1.79 mg, 10 μmol) and 2,6-diethynylpyridine A (0.67 mg, 5 μmol) in DMSO (150 μL). This solution was mixed with a solution of TBTA (5.1 mg, 0.96 μmol) and sodium ascorbate (6.4 mg, 32 μmol) in DMSO (95 μL). The click reaction was initiated by the addition of 20 Mm copper sulfate (5 μL) with agitation. The extent of reaction was analyzed by TLC (94:5:1 ethyl acetate: methanol: acetic acid) and the reaction was complete in 5 minutes based on the consumption of azide and alkyne. The reaction mixture volume was brought to 1 Ml with DMSO and 0.5 M EDTA (100 μL). Solids were isolated and dissolved in additional DMSO. The DMSO solutions were combined and purified by flash chromatography as described above in Materials and Methods. The product formed a glassy solid upon rotary evaporation in a 50 to 75% yield. ¹H NMR (300 MHz, DMSO-d6) δ ppm 3.29 (2H, br. S., (Q(18)H and O(33)H)), 7.17-7.29 (4H,m, (C(7)H, C(11)H, C(28)H, C(32)H)) 7.85 (2H, d, J=8.11 (C(10)H and C(31)H)), 8.06 (3H, s, (C(15)H, C(16)H, C(17)H)) 9.36 (2H, s, (C(5)H and C(25)H)).

### Example 2

### Synthesis of 4,4′-(pyridine-3,5-diylbis(1H-1,2,3-triazole-4,1-diyl))bis(2-hydroxybenzoic acid)

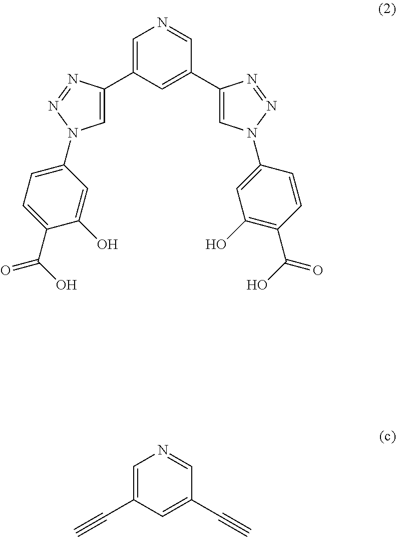

Compound 2 was prepared using 4-azidosalicylic acid B and 3,5-diethynylpyridine C according to the method of Example 1. ¹H NMR (300 MHz, DMSO-d6) δ ppm 3.29 (2H, br. S., (Q(18)H and Q(33)H)) 7.17-7.29 (4H, m, (C(7)H, C(11)H, C(28)H, C(31)H)) 7.85 (2H, d, J=8.11 Hz, (C(10)H and C(31)H)) 8.06 (3H, s, (C(13)H, C(15)H, C(17)H)) 9.36 (2H,s, (C(5)H and C(25)H)).

### Example 3

### Synthesis of 4,4′-(1,3-phenylenebis(1H-1,2,3-triazole-4,1-diyl))bis(2-hydroxybenzoic acid)

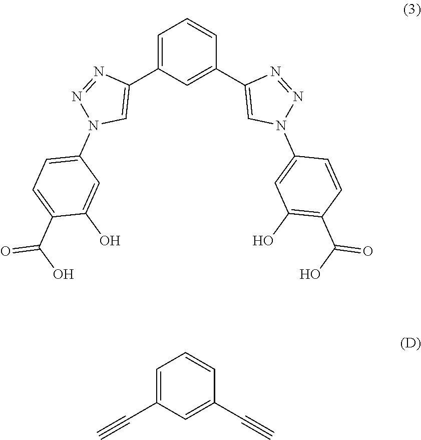

Compound 3 was prepared using 4-azidosalicylic acid B and 1,3-diethynylbenzene D according to the method of Example 1. ¹H NMR (300 MHz, DMSO-d6) δ ppm 3.29 (2H, br. S., (O(18)H and O(33)H)) 7.16-7.27 (4H, m, (C(7)H, C(11)H, C(28)H, C(32)H)) 7.57-7.67 (1H,m, (C(16)H)) 7.80-7.88 (2H,m, (C(10)H and C(31)H)) 7.94 (2H,d, J=7.63 Hz, (C(15)H and C(17)H) 8.57(1H,s, (C(13)H)) 9.35 (2H,s, (C(5)H and C(25)H).

### Example 4

### Synthesis of 4,4′-((9H-carbazole-3,6-diyl)bis(1H-1,2,3-triazole-4,1-diyl))bis(2-hydroxybenzoic acid)

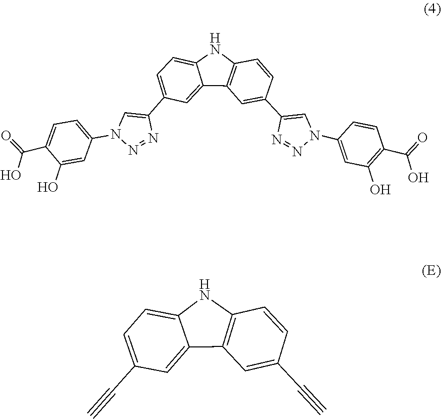 [SMILES: O=C(O)c1ccc(-n2cc(-c3ccc4[nH]c5ccc(-c6cn(-c7ccc(C(=O)O)c(O)c7)nn6)cc5c4c3)nn2)cc1O ; C#Cc1ccc2[nH]c3ccc(C#C)cc3c2c1]

Compound 4 was prepared using 4-azidosalicylic acid B and 3,6-diethynylcarbazole E according to the method of Example 1. ¹H NMR (300 MHz, DMSO-d6) δ ppm 3.29 (2H, br. S., (Q(25)H and Q(40)H)) 7.16-7.27 (4H, m, (C(7)H, C(11)H, C(35)H, C(39)H)) 7.61 (2H,d, J=8.34 Hz, (C(10)H and C(38)H)) 7.84 (2H,d, J=7.87 Hz, (C(22)H and C(24)H)) 8.02 (2H,d, J=8.34 Hz, (C(21)H and C(23)H)) 8.79 (2H,s, (C(13)H and C(19)H)) 9.28 (2H,s, (C(5)H and C(32)H)) 11.53 (1H,s, (N(16)H).

### Example 5

### Synthesis of 4,4′-((9H-carbazole-3,6-diyl)bis(1H-1,2,3-triazole-4,1-diyl))dianiline

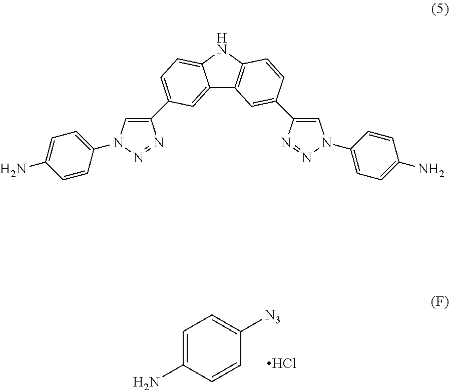 [SMILES: Nc1ccc(-n2cc(-c3ccc4[nH]c5ccc(-c6cn(-c7ccc(N)cc7)nn6)cc5c4c3)nn2)cc1 ; [N-]=[N+]=Nc1ccc(N)cc1 ; Cl]

Compound 5 was prepared using 4-azidoaniline hydrochloride F and 3,6-diethynylcarbazole E according to the method of Example 1.

### Example 6

### Synthesis of 4,4′-((9H-carbazole-3,6-diyl)bis(1H-1,2,3-triazole-4,1-diyl))dibenzoic acid

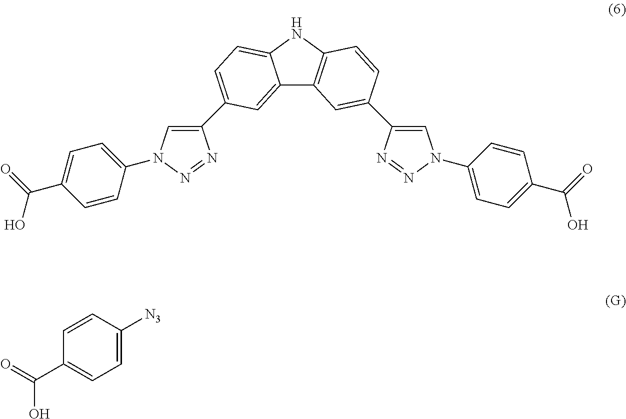 [SMILES: O=C(O)c1ccc(-n2cc(-c3ccc4[nH]c5ccc(-c6cn(-c7ccc(C(=O)O)cc7)nn6)cc5c4c3)nn2)cc1 ; [N-]=[N+]=Nc1ccc(C(=O)O)cc1]

Compound 6 was prepared using 4-azidobenzoic acid G and 3,6-diethynylcarbazole E according to the method of Example 1.

### Example 7

### Synthesis of 3,6-bis(1-(4-methoxyphenyl)-1H-1,2,3-triazol-4-yl)-9H-carbazole

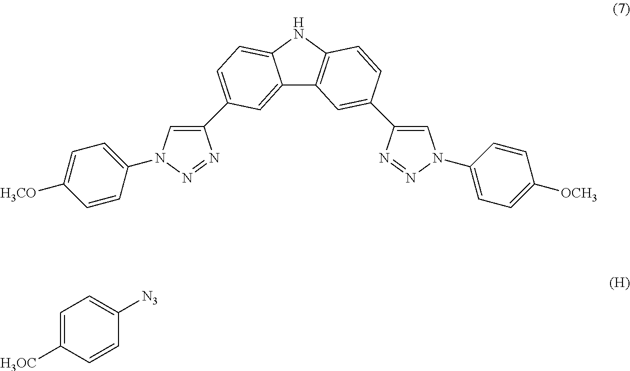

Compound 7 was prepared using 4-azidoanisole H and 3,6-diethynylcarbazole E according to the method of Example 1.

### Example 8

### Synthesis of dimethyl 2,2′-((9H-carbazole-3,6-diyl)bis(1H-1,2,3-triazole-4,1-diyl))diacetate

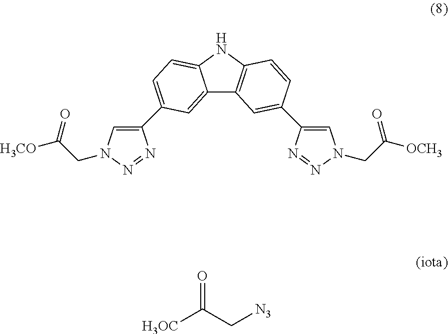

Compound 8 was prepared using methyl azidoacetate iota and 3,6-diethynylcarbazole E according to the method of Example 1.

### Example 9

### Synthesis of 4,4′-((4-methoxypyridine-2,6-diyl)bis(1H-1,2,3-triazole-4,1-diyl))bis(2-hydroxybenzoic acid)

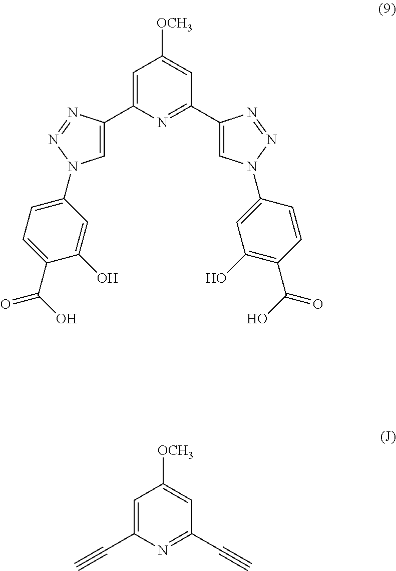 [SMILES: COc1cc(-c2cn(-c3ccc(C(=O)O)c(O)c3)nn2)nc(-c2cn(-c3ccc(C(=O)O)c(O)c3)nn2)c1 ; C#Cc1cc(OC)cc(C#C)n1]

Preparation of compound 9 started with the synthesis of 4-methoxy-2,6-diethynylpyridine J from 4-methoxy-2,6-dibromopyridine and ethynyltrimethylsilane using conditions described by Sonogashira (Organomet. Chem., 653: 46-49 (2002). doi:10.1016/s0022-328x(02)01158-0). Synthesis of compound 9 was completed by clicking 4-azidosalicylic acid B and compound J according to the method of Example 1.

### Example 10

### Synthesis of 4,4′-((4-carboxypyridine-2,6-diyl)bis(1H-1,2,3-triazole-4,1-diyl))bis(2-hydroxybenzoic acid)

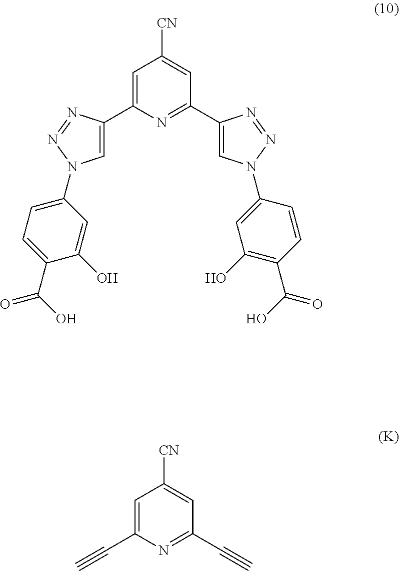 [SMILES: N#Cc1cc(-c2cn(-c3ccc(C(=O)O)c(O)c3)nn2)nc(-c2cn(-c3ccc(C(=O)O)c(O)c3)nn2)c1 ; C#Cc1cc(C#N)cc(C#C)n1]

Preparation of compound 10 started with the synthesis of 4-cyano-2,6-diethynylpyridine K from 4-cyano-2,6-dibromopyridine and ethynyltrimethylsilane using conditions described by Sonogashira (Organomet. Chem., 653: 46-49(2002). doi:10.1016/s0022-328x(02)01158-0). Synthesis of compound 10 was completed by clicking 4-azidosalicylic acid B and compound K according to the method of Example 1.

### Example 11

### Synthesis of 4,4′-((4-nitropyridine-2,6-diyl)bis(1H-1,2,3-triazole-4,1-diyl))bis(2-hydroxybenzoic acid)

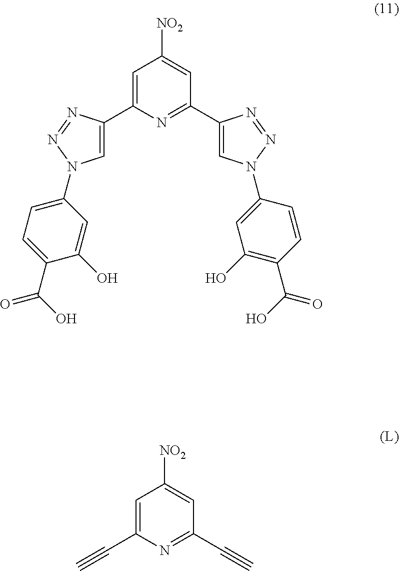 [SMILES: O=C(O)c1ccc(-n2cc(-c3cc([N+](=O)[O-])cc(-c4cn(-c5ccc(C(=O)O)c(O)c5)nn4)n3)nn2)cc1O ; C#Cc1cc([N+](=O)[O-])cc(C#C)n1]

Preparation of compound 11 started with synthesis of 4-nitro-2,6-diethynylpyridine L from 4-nitro-2,6-dibromopyridine and ethynyltrimethylsilane using conditions described by Sonogashira (Organomet. Chem., 653: 46-49 (2002). doi:10.1016/s0022-328x(02)01158-0). Synthesis of compound 11 was completed by clicking 4-azidosalicylic acid B and compound L according to the method of Example 1.

### Example 12

### Synthesis of 5,5′-((4-cyanopyridine-2,6-diyl)bis(1H-1,2,3-triazole-4,1-diyl))bis(2-hydroxybenzoic acid)

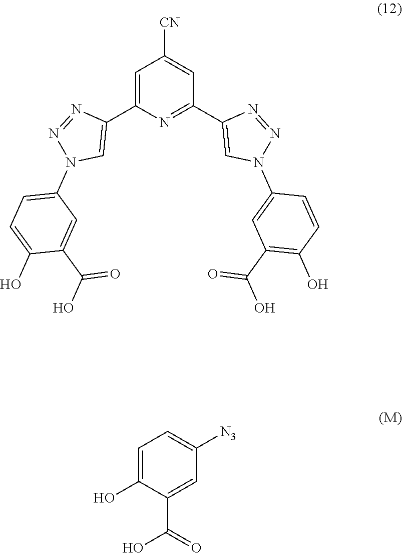

Compound 12 was prepared using 5-azidosalicylic acid M and 4-cyano-2,6-diethynylpyridine K according to the method of Example 10.

### Example 13

### Synthesis of 4,4′-((4-methylpyridine-2,6-diyl)bis(1H-1,2,3-triazole-4,1-diyl))bis(2-hydroxybenzoic acid)

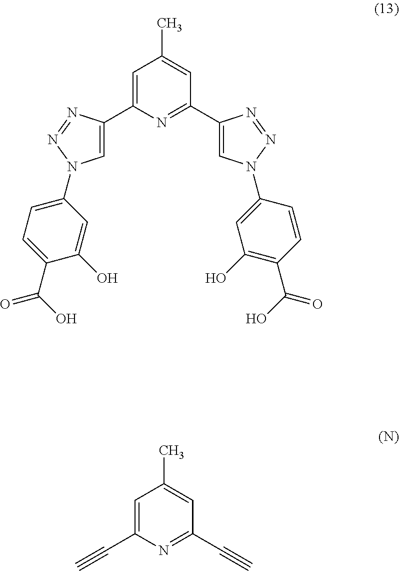 [SMILES: Cc1cc(-c2cn(-c3ccc(C(=O)O)c(O)c3)nn2)nc(-c2cn(-c3ccc(C(=O)O)c(O)c3)nn2)c1 ; C#Cc1cc(C)cc(C#C)n1]

Compound 13 started with synthesis of 4-methyl-2,6-diethynylpyridine N from 4-methyl-2,6-dichloropyridine and ethynyltrimethylsilane using conditions described by Sonogashira (Organomet. Chem., 653: 46-49 (2002). doi:10.1016/s0022-328x(02)01158-0). Synthesis of compound 11 was completed by clicking 4-azidosalicylic acid B and 4-methyl-2,6-diethynylpyridine N according to the method of Example 1.

### Example 14

### Synthesis of 4,4′-((4-(ethoxycarbonyl)pyridine-2,6-diyl)bis(1H-1,2,3-triazole-4,1-diyl))bis(2-hydroxybenzoic acid)

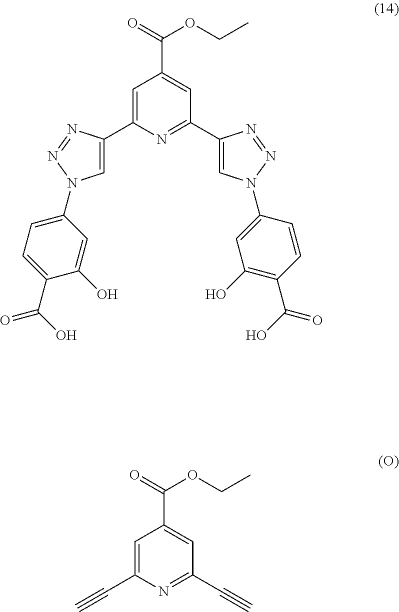 [SMILES: CCOC(=O)c1cc(-c2cn(-c3ccc(C(=O)O)c(O)c3)nn2)nc(-c2cn(-c3ccc(C(=O)O)c(O)c3)nn2)c1 ; C#Cc1cc(C(=O)OCC)cc(C#C)n1]

Compound 14 started with synthesis of ethyl 2,6-diethynylpyridine-4-carboxylate O from ethyl 2,6-dibromopyridine-4-carboxylate and ethynyltrimethylsilane using conditions described by Sonogashira (Organomet. Chem., 653: 46-49 (2002). doi:10.1016/s0022-328x(02)01158-0). Synthesis of compound 14 was completed by clicking 4-azidosalicylic acid B and ethyl 2,6-diethynylpyridine-4-carboxylate O according to the method of Example 1.

### Example 15

### Synthesis of 5,5′-((4-(ethoxycarbonyl)pyridine-2,6-diyl)bis(1H-1,2,3-triazole-4,1-diyl))bis(2-hydroxybenzoic acid)

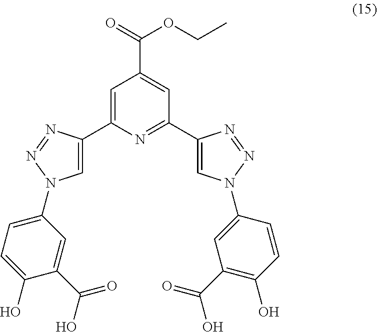 [SMILES: CCOC(=O)c1cc(-c2cn(-c3ccc(O)c(C(=O)O)c3)nn2)nc(-c2cn(-c3ccc(O)c(C(=O)O)c3)nn2)c1]

Compound 15 was prepared using 5-azidosalicylic acid M and ethyl 2,6-diethynylepyridine-4-carboxylate O clicked according to the method of Example 14.

### Example 16

### Synthesis of 4,4′-((4-(methoxycarbonyl)pyridine-2,6-diyl)bis(1H-1,2,3-triazole-4,1-diyl))bis(2-hydroxybenzoic acid)

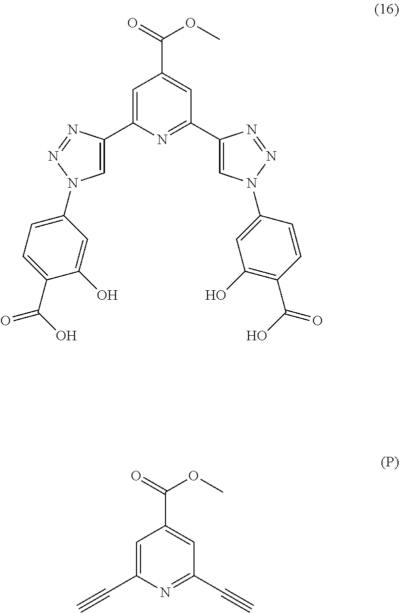 [SMILES: COC(=O)c1cc(-c2cn(-c3ccc(C(=O)O)c(O)c3)nn2)nc(-c2cn(-c3ccc(C(=O)O)c(O)c3)nn2)c1 ; C#Cc1cc(C(=O)OC)cc(C#C)n1]

Compound 16 started with synthesis of methyl 2,6-diethynylpyridine-4-carboxylate P from methyl 2,6-dichloropyridine-4-carboxylate and ethynyltrimethylsilane using conditions described by Sonogashira (Organomet. Chem., 653: 46-49(2002). doi:10.1016/s0022-328x(02)01158-0). Synthesis of compound 16 was completed by clicking 4-azidosalicylic acid B and methyl 2,6-diethynylpyridine-4-carboxylate P according to the method of Example 1.

### Example 17

### Synthesis of 4,4′-((4-(ethylcarbamoyl)pyridine-2,6-diyl)bis(1H-1,2,3-triazole-4,1-diyl))bis(2-hydroxybenzoic acid)

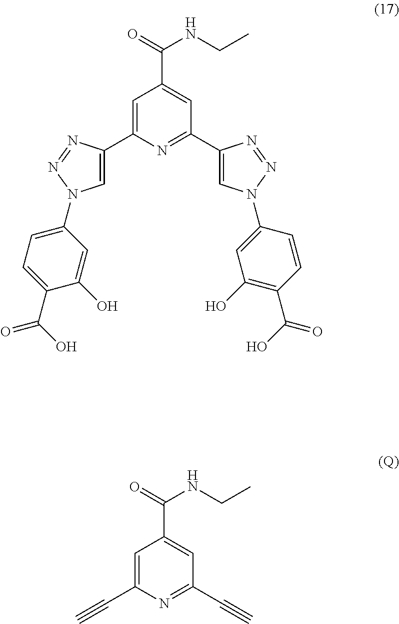 [SMILES: CCNC(=O)c1cc(-c2cn(-c3ccc(C(=O)O)c(O)c3)nn2)nc(-c2cn(-c3ccc(C(=O)O)c(O)c3)nn2)c1 ; C#Cc1cc(C(=O)NCC)cc(C#C)n1]

Synthesis of compound 17 commenced with mixing 2,6-dibromo-4-pyridine carboxylic acid (0.2 g, 0.71 mmol), DIPEA (0.18 g, 1.42 mmol) and HATU (0.27 g, 0.71 mmol) in DMF (900 μl). Ethyl amine (0.154 ml, 1.78 mmol) was added immediately and mixed for 1 hour. The reaction was completed by TLC and purified by flash chromatography on silica gel using a gradient of ethyl acetate/hexane. N-ethyl-2,6-dibromo-4-carboxamide was isolated as a yellow solid in 69% yield. N-ethyl-2,6-diethynl-4-carboxamide Q was made with ethyltrimethylsilane using the Sonogashira method described in Example 16. Synthesis of compound 17 was completed by clicking 4-azidosalicylic acid B and N-ethyl-2,6-diethynl-4-carboxamide Q according to the method of Example 1.

### Example 18

### Synthesis of 4,4′-((4-(methylcarbamoyl)pyridine-2,6-diyl)bis(1H-1,2,3-triazole-4,1-diyl))bis(2-hydroxybenzoic acid)

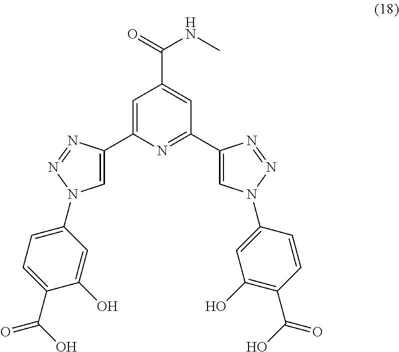 [SMILES: CNC(=O)c1cc(-c2cn(-c3ccc(C(=O)O)c(O)c3)nn2)nc(-c2cn(-c3ccc(C(=O)O)c(O)c3)nn2)c1]

Compound 18 was prepared with methyl amine to form the amide according to the method of Example 17.

### Example 19

### Synthesis of 4,4′-((4-carbamoylpyridine-2,6-diyl)bis(1H-1,2,3-triazole-4,1-diyl))bis(2-hydroxybenzoic acid)

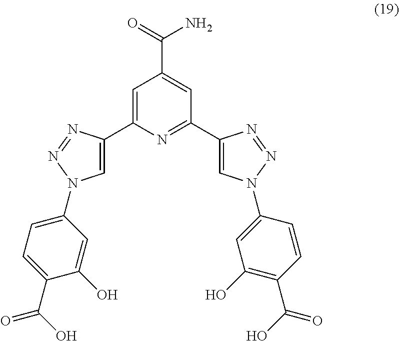 [SMILES: NC(=O)c1cc(-c2cn(-c3ccc(C(=O)O)c(O)c3)nn2)nc(-c2cn(-c3ccc(C(=O)O)c(O)c3)nn2)c1]

Compound 19 was prepared with ammonia to form the amide according to the method of Example 17.

### Example 20

### Synthesis of 4,4′-(pyrazine-2,6-diylbis(1H-1,2,3-triazole-4,1-diyl))bis(2-hydroxybenzoic acid)

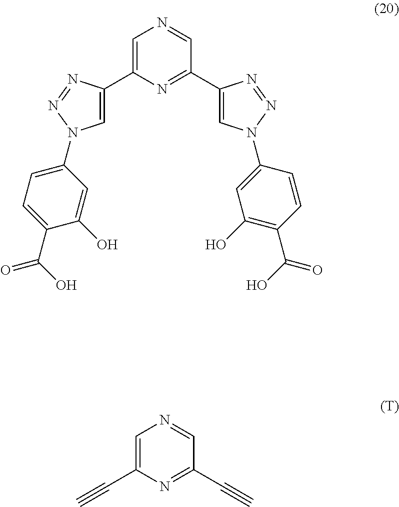

Compound 20 was started with synthesis of 2,6-diethynylpyrazine T from 2,6-dichloropyrazine and ethynyltrimethylsilane using Sonogashira method described by Bhowmick, S. et al. (App. Organomet. Chem. 31(12):e3824 (2017)). Synthesis of compound 20 was completed by clicking 4-azidosalicylic acid B and 2,6-diethynylpyrazine T according to the method of Example 1.

### Example 21

### Synthesis of 4,4′-((4-(ethylcarbamoyl)pyridine-2,6-diyl)bis(1H-1,2,3-triazole-4,1-diyl))bis(2-(trifluoromethyl)benzoic acid)

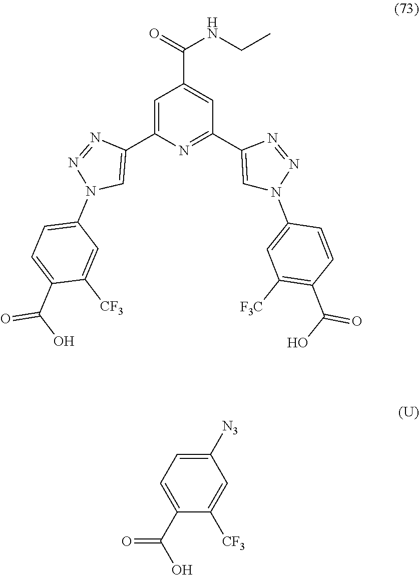 [SMILES: CCNC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1 ; [N-]=[N+]=Nc1ccc(C(=O)O)c(C(F)(F)F)c1]

Synthesis of compound 73 started with diazotization of 4-amino-2-(trifluoromethyl)benzoic acid with sodium nitrite and sulfuric acid followed by nucleophilic displacement with azide (Org. Synth. 1942, 22, 96) to form 4-azido-2-(trifluoromethyl)benzoic acid (L) which was purified via flash chromatography. Synthesis of compound 73 was completed by clicking 4-azido-2-(trifluoromethyl)benzoic acid U and N-ethyl-2,6-diethynl-4-carboxamide Q according to Example 1.

### Example 22

### Synthesis of 7,7′-((4-(ethylcarbamoyl)pyridine-2,6-diyl)bis(1H-1,2,3-triazole-4,1-diyl))bis(2-hydroxy-1,8-naphthyridine-4-carboxylic acid)

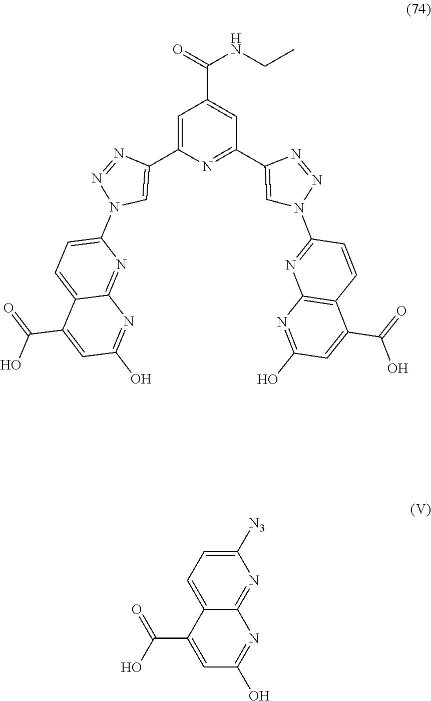 [SMILES: CCNC(=O)c1cc(-c2cn(-c3ccc4c(C(=O)O)cc(O)nc4n3)nn2)nc(-c2cn(-c3ccc4c(C(=O)O)cc(O)nc4n3)nn2)c1 ; [N-]=[N+]=Nc1ccc2c(C(=O)O)cc(O)nc2n1]

Synthesis of compound 74 started with diazotization of 7-amino-2-hydroxy-1,8-naphthyridine-4-carboxylic acid with sodium nitrite and sulfuric acid followed by nucleophilic displacement with azide (Org. Synth. 1942, 22, 96) to form 7-azido-2-hydroxy-1,8-naphthyridine-4-carboxylic acid (V) which was purified via flash chromatography. Synthesis of compound 74 was completed by clicking 7-azido-2-hydroxy-1,8-naphthyridine-4-carboxylic acid V and N-ethyl-2,6-diethynl-4-carboxamide Q according to Example 1.

### Example 23

### Synthesis of 4-(4-(3-(1-(4-carboxyphenyl)-1H-1,2,3-triazol-4-yl)phenyl)-1H-1,2,3-triazol-1-yl)-2-hydroxybenzoic acid

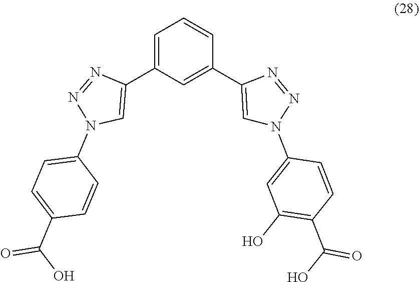 [SMILES: O=C(O)c1ccc(-n2cc(-c3cccc(-c4cn(-c5ccc(C(=O)O)c(O)c5)nn4)c3)nn2)cc1]

Synthesis of compound 28 was completed in two steps. First, 2,6-diethynylbenzene D was clicked with half the amount of 4-azidosalicylic acid B according to the method of Example 3 to make 4-(4-(3-ethynylphenyl)-1H-1,2,3-triazol-1-yl)-2-hydroxybenzoic acid. The second step was clicking 4-azidobenzoic acid G according to the method of Example 1 to give 4-(4-(3-(1-(4-carboxyphenyl)-1H-1,2,3-triazol-4-yl)phenyl)-1H-1,2,3-triazol-1-yl)-2-hydroxybenzoic acid 28.

### Example 24

### Synthesis of 4-(4-(4-cyanopyridin-2-yl)-1H-1,2,3-triazol-yl)-2-hydroxybenzoic acid

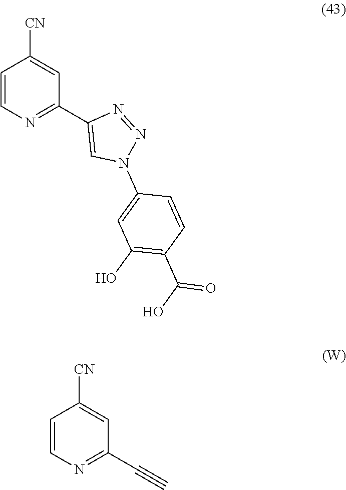

Preparation of compound 43 started with synthesis of 4-cyano-2-ethynylpyridine W from 4-cyano-2-chloropyridine and ethynyltrimethylsilane using conditions described by Sonogashira (Organomet. Chem., 653: 46-49 (2002). doi:10.1016/s0022-328x(02)01158-0). Synthesis of compound 43 was completed by clicking 4-azidosalicylic acid B and compound W according to the method of Example 1.

### Example 25

### Synthesis of 4,4′-((pyridine-2,6-diyl)bis(5-iodo-1H-1,2,3-triazole-4,1-diyl))bis(2-hydroxybenzoic acid)

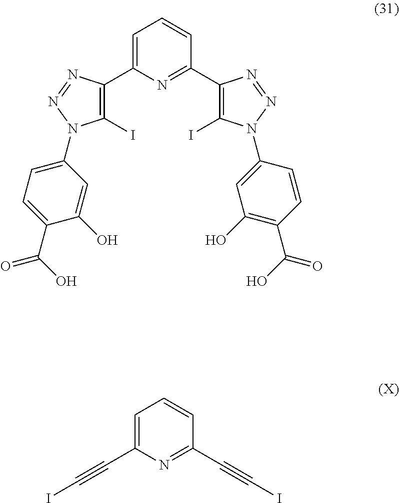 [SMILES: O=C(O)c1ccc(-n2nnc(-c3cccc(-c4nnn(-c5ccc(C(=O)O)c(O)c5)c4I)n3)c2I)cc1O ; IC#Cc1cccc(C#CI)n1]

Preparation of compound 31 started with the synthesis of 2,6-bis(iodoethynyl)pyridine X from 2,6-diethynylpyridine A following the method of Tepper et. al, (Org. Lett., 2015, 17 (23), pp 5740-574) which involved treatment with n-iodosuccinimide and silver nitrate and isolation by flash chromatography. Synthesis of compound 31 was completed by clicking 4-azidosalicylic acid B and compound X according to the method of Example 1.

### Example 26

### Synthesis of 4,4′-((3,5-dimethylpyridine-2,6-diyl)bis(1H-1,2,3-triazole-4,1-diyl))bis(2-hydroxybenzoic acid)

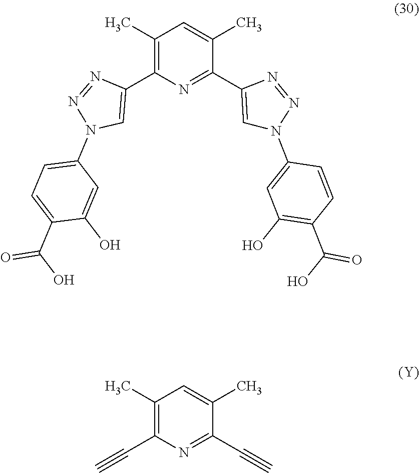 [SMILES: Cc1cc(C)c(-c2cn(-c3ccc(C(=O)O)c(O)c3)nn2)nc1-c1cn(-c2ccc(C(=O)O)c(O)c2)nn1 ; C#Cc1nc(C#C)c(C)cc1C]

Compound 30 was started with synthesis of 2,6-diethynyl-3,5-dimethylpyridine Y from 2,6-dibromo-3,5-dimethylpyridine and ethynyltrimethylsilane using Sonogashira method described by Bhowmick, S. et al. (App. Organomet. Chem. 31(12):e3824 (2017)). Synthesis of compound 30 was completed by clicking 4-azidosalicylic acid B and 2,6-diethynyl-3,5-dimethylpyridine Y according to the method of Example 1.

### Example 27

### Synthesis of 4,4′-((9-acetyl-9H-carbazole-3,6-diyl)bis(1H-1,2,3-triazole-4,1-diyl))bis(2-hydroxybenzoic acid)

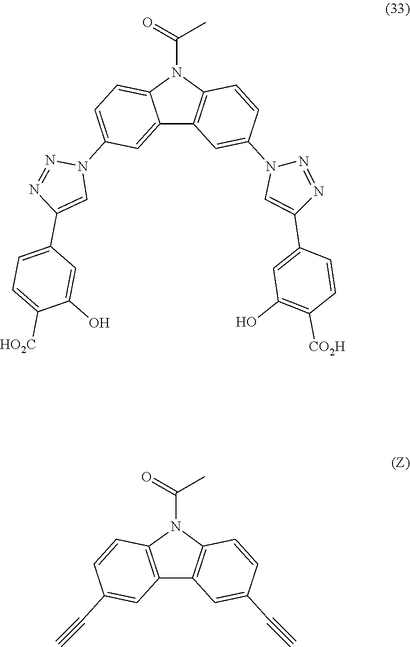 [SMILES: CC(=O)n1c2ccc(-n3cc(-c4ccc(C(=O)O)c(O)c4)nn3)cc2c2cc(-n3cc(-c4ccc(C(=O)O)c(O)c4)nn3)ccc21 ; C#Cc1ccc2c(c1)c1cc(C#C)ccc1n2C(C)=O]

Compound 33 was started with synthesis of 9-acetyl-3,6-diethynylcarbazole Z from 9-acetyl-3,6-diiodocarbazole and ethynyltrimethylsilane using Sonogashira method described by Bhowmick, S. et al. (App. Organomet. Chem. 31(12):e3824 (2017)). Synthesis of compound 33 was completed by clicking 4-azidosalicylic acid B and 9-acetyl-3,6-diethynylcarbazole Z according to the method of Example 1.

### Example 28

### Synthesis of 4,4′-(pyridine-2,6-diylbis(1H-1,2,3-triazole-4,1-diyl))bis(N,2-dihydroxybenzamide)

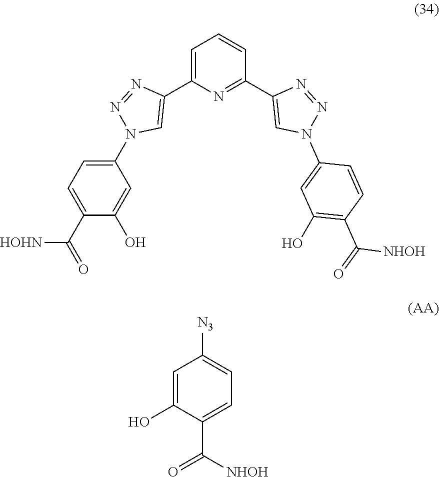

Synthesis of compound 34 commenced with mixing N-hydroxysuccinimide 4-azidosalicylate (40 mg, 0.145 mmol) in DMF (72 μl) to this was added hydroxyl amine hydrochloride (30 mg, 0.43 mmol) in water (72 μl) and mixed overnight. Product was detected by TLC and the reaction was purified by flash chromatography on silica gel using a gradient of methylene chloride and methanol. 4-Azido-N,2-dihydroxybenzamide AA was isolated in 57% yield. Synthesis of compound 34 was completed by clicking 4-azido-N,2-dihydroxybenzamide AA and 2,6-diethynylpyridine A according to the method of Example 1.

### Example 29

### Synthesis of 5-(4-(6-(4-(3-carboxy-4-hydroxy-5-methylphenyl)-1H-1,2,3-triazol-1-yl)-4-(methoxycarbonyl)pyridin-2-yl)-1H-1,2,3-triazol-1-yl)-2-hydroxy-3-methylbenzoic acid

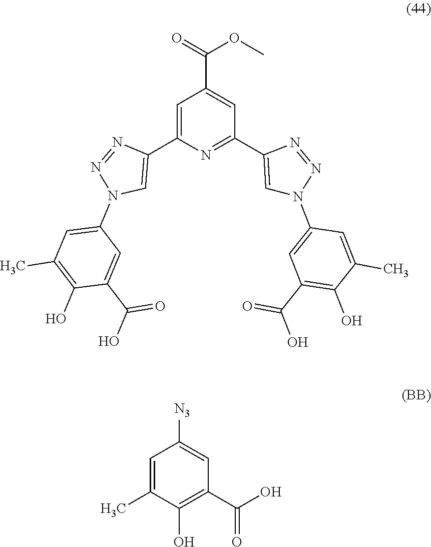

Synthesis of compound 44 started with diazotization of 5-amino-2-hydroxy-3-methylbenzoic acid with sodium nitrite and sulfuric acid followed by nucleophilic displacement with azide (Org. Synth. 1942, 22, 96) to form 5-azido-2-hydroxy-3-methylbenzoic acid (BB) which was purified via flash chromatography. Synthesis of compound 44 was completed by clicking 5-azido-2-hydroxy-3-methylbenzoic acid BB with 2,6-diethynylpyridine-4-carboxylate P according to Example 1.

### Example 30

### Synthesis of 4,4′-((4-(but-3-yn-1-ylcarbamoyl)pyridine-2,6-diyl)bis(1H-1,2,3-triazole-4,1-diyl))bis(2-hydroxybenzoic acid)

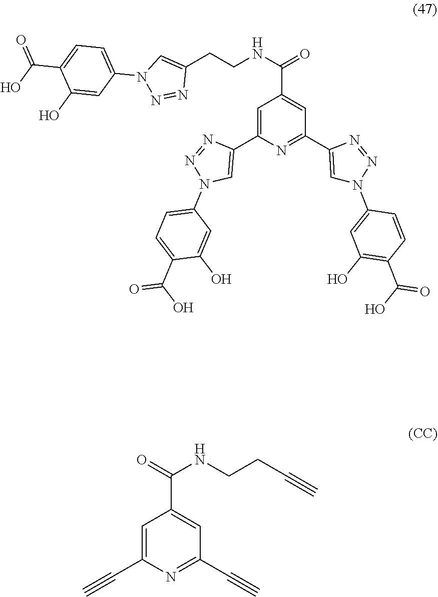

Synthesis of compound 47 commenced with mixing 2,6-dibromo-4-pyridine carboxylic acid (0.2 g, 0.71 mmol), DIPEA (0.18 g, 1.42 mmol) and HATU (0.27 g, 0.71 mmol) in DMF (900 μl). Butynyl amine (0.154 ml, 1.78 mmol) was added immediately and mixed for 1 hour. The reaction was completed by TLC and purified by flash chromatography on silica gel using a gradient of ethyl acetate/hexane. N-(but-3-yn-1-yl)-2,6-dibromoisonicotinamide was isolated as a solid. N-(but-3-yn-1-yl)-2,6-diethynylisonicotinamide CC was made with ethyltrimethylsilane using the Sonogashira method described in Example 16. Synthesis of compound 47 was completed by clicking 4-azidosalicylic acid B and N-(but-3-yn-1-yl)-2,6-diethynylisonicotinamide CC according to the method of Example 1.

### Example 31

### Synthesis of 4,4′-(naphthalene-2,7-diylbis(1h-1,2,3-triazole-4,1-diyl))bis(2-hydroxybenzoic acid)

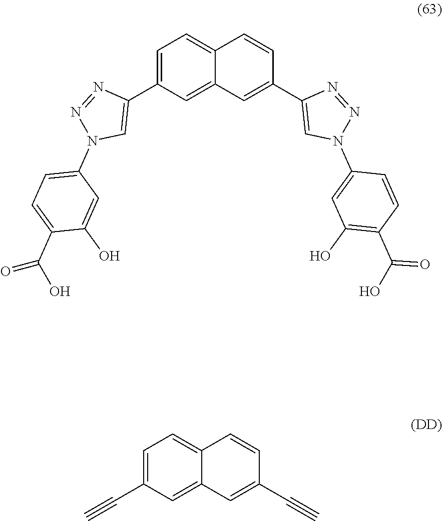

Compound 63 was started with synthesis of 2,7-diethynylnaphthalene DD from 2,7-dibromonaphthalene and ethynyltrimethylsilane using the Sonogashira method described by Bhowmick, S. et al. (App. Organomet. Chem. 31(12):e3824 (2017)). Synthesis of compound 63 was completed by clicking 4-azidosalicylic acid B and 2,7-diethynylnaphthalene DD according to the method of Example 1.

### Example 32

### Synthesis of 4,4′-(naphthalene-2,3-diylbis(1H-1,2,3-triazole-4,1-diyl))bis(2-hydroxybenzoic acid)

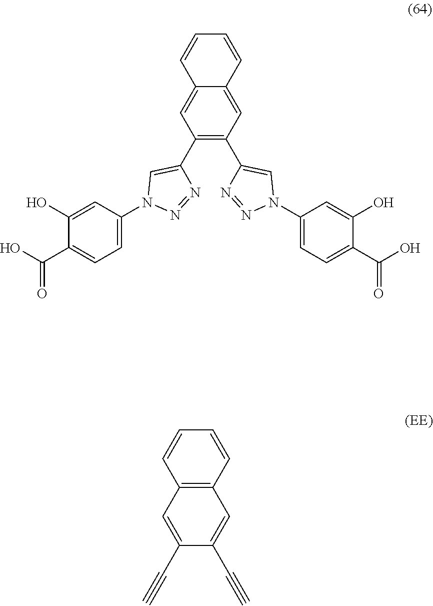

Compound 64 was started with synthesis of 2,3-diethynylnaphthalene EE from 2,3-dibromonaphthalene and ethynyltrimethylsilane using the Sonogashira method described by Bhowmick, S. et al. (App. Organomet. Chem. 31(12):e3824 (2017). Synthesis of compound 64 was completed by clicking 4-azidosalicylic acid B and 2,3-diethynylnaphthalene EE according to the method of Example 1.

### Example 33

### Synthesis of 4,4′,4″,4′″-((((butane-1,4-diylbis(azanediyl))bis(carbonyl))bis(pyridine-4,2,6-triyl))tetrakis(1H-1,2,3-triazole-4,1-diyl))tetrakis(2-hydroxybenzoic acid)

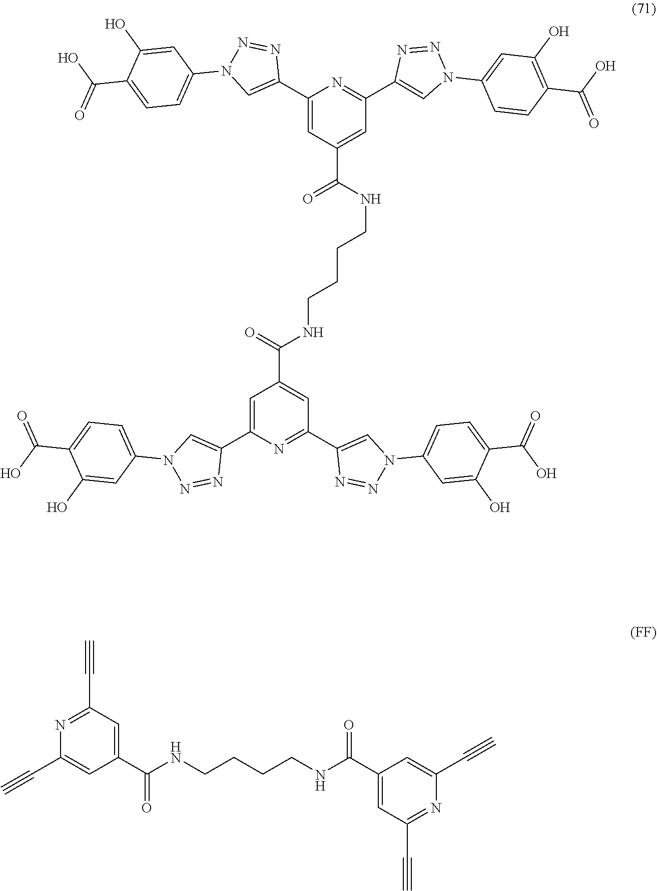

Compound 71 was started with synthesis of 2,6-diethynyl-4-pyridine carboxylic acid from 2,6-dibromo-4-pyridine carboxylic acid and ethynyltrimethylsilane using the Sonogashira method described by Bhowmick, S. et al. (App. Organomet. Chem. 31(12):e3824 (2017)). 2,6-Diethynyl-4-pyridine carboxylic acid was treated with HATU, DIPEA and 1,4-diaminobutane to give N,N′-(butane-1,4-diyl)bis(2,6-diethynylisonicotinamide) (FF) after isolation by flash chromatography. Synthesis of compound 71 was completed by clicking 4-azidosalicylic acid B and N,N′-(butane-1,4-diyl)bis(2,6-diethynylisonicotinamide) (FF) according to the method of Example 1.

### Example 34

### Synthesis of 4,4′-((4-(ethylcarbamoyl)pyridine-2,6-diyl)bis(1H-1,2,3-triazole-4,1-diyl))bis(3,5,6-trichloropicolinic acid)

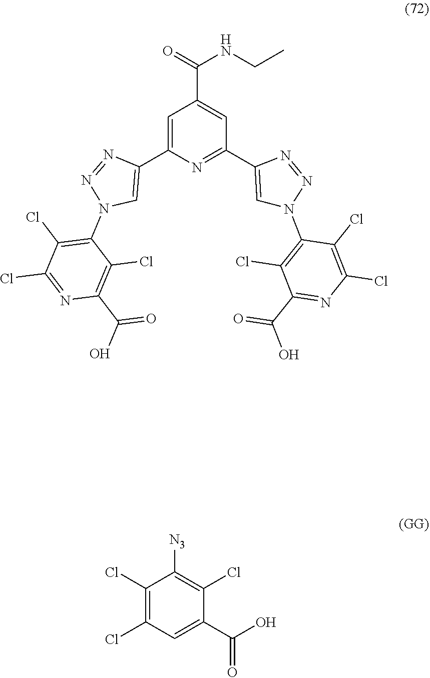

Synthesis of compound 72 started with diazotization of 4-Anino-35,6-trichloropyridine 2-carboxylic acid with sodium nitrite and sulfuric acid followed by nucleophilic displacement with azide (Org. Synth. 1942, 22, 96) to form 4-Azido-3,5,6-trichloropyridine-2-carboxylic acid (GG) which was purified via flash chromatography. Synthesis of compound 72 was completed by clicking 4-Azido-3,5,6-trichloropyridine-2-carboxylic acid GG with N-ethyl-2,6-diethynl-4-carboxamide Q according to Example 1.

### Example 35

### Synthesis of 4,4′-((4-(methylcarbamoyl)pyridine-2,6-diyl)bis(1H-1,2,3-triazole-4,1-diyl))bis(2-(trifluoromethyl)benzoic acid)

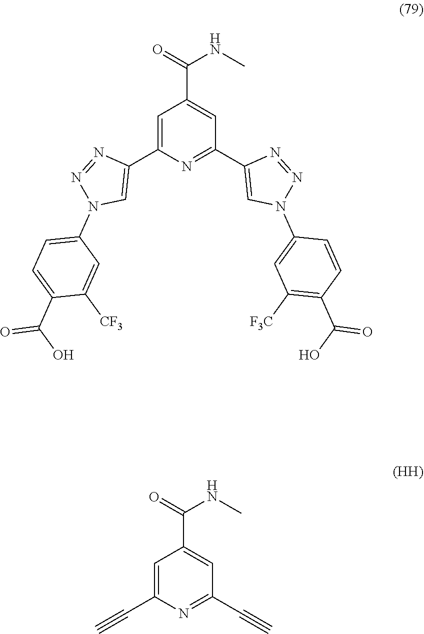 [SMILES: CNC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1 ; C#Cc1cc(C(=O)NC)cc(C#C)n1]

N-Methyl-2,6-diethynylpyridine-4-carboxamide HH was prepared according to Example 17 using HATU coupling of 2,6-diethynylpyridine-4-carboxylic acid with methylamine. Compound 79 was made using the copper click method in Example 1 using diethynyl HH and azide U. Compound 79 Mass Spec (ESI Negative Mode): Calcd for C₂₇H₁₆F₆N₈O₅ 646.47; Found: 645 [M−H⁺].

### Example 36

### Synthesis of 4,4′-((4-(morpholine-4-carbonyl)pyridine-2,6-diyl)bis(1H-1,2,3-triazole-4,1-diyl))bis(2-(trifluoromethyl)benzoic acid)

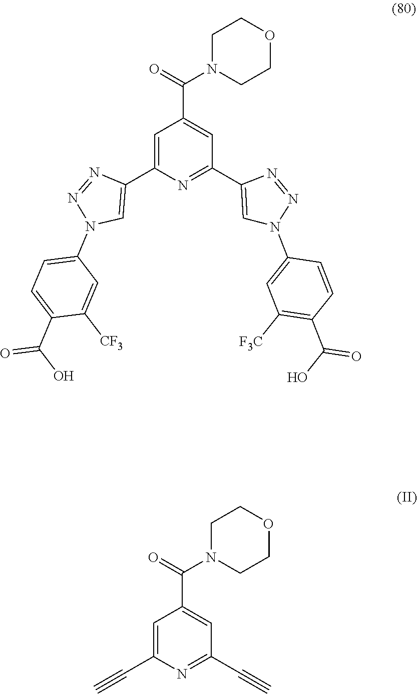 [SMILES: O=C(O)c1ccc(-n2cc(-c3cc(C(=O)N4CCOCC4)cc(-c4cn(-c5ccc(C(=O)O)c(C(F)(F)F)c5)nn4)n3)nn2)cc1C(F)(F)F ; C#Cc1cc(C(=O)N2CCOCC2)cc(C#C)n1]

N-Morpholino-2,6-diethynylpyridine-4-carboxamide II was prepared according to Example 17 using HATU coupling of 2,6-diethynylpyridine-4-carboxylic acid with morpholine. Compound 80 was made using the copper click method in Example 1 using diethynyl II and azide U. Compound 80 Mass Spec (ESI Negative Mode): Calcd for C30H20F6N8O6 702.53; Found: 701.1 [M−H+]

### Example 37

### Synthesis of 4,4′-((4-(diethylcarbamoyl)pyridine-2,6-diyl)bis(1H-1,2,3-triazole-4,1-diyl))bis(2-(trifluoromethyl)benzoic acid)

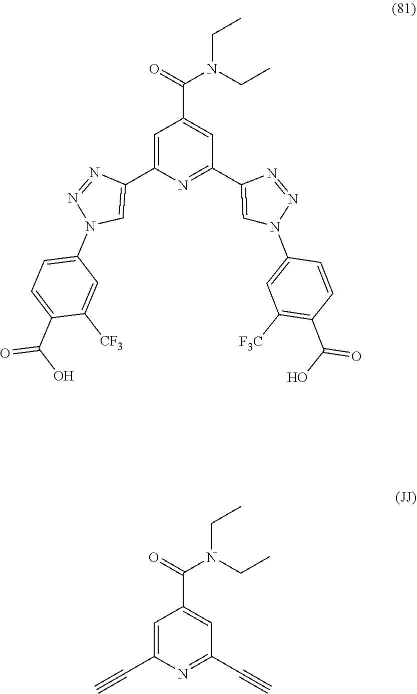 [SMILES: CCN(CC)C(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1 ; C#Cc1cc(C(=O)N(CC)CC)cc(C#C)n1]

N,N-diethylamino-2,6-diethynylpyridine-4-carboxamide JJ was prepared according to Example 17 using the HATU coupling of 2,6-diethynylpyridine-4-carboxylic acid with N,N-diethylamine. Compound 81 was made using the copper click method in Example 1 using diethynyl JJ and azide U. Compound 81 Mass Spec (ESI Negative Mode): Calcd for C₃₀H₂₂F₆N₈O₅ 688.55; Found: 687 [M−H⁺]

### Example 38

### Synthesis of 4,4′-((4-carbamoylpyridine-2,6-diyl)bis(1H-1,2,3-triazole-4,1-diyl))bis(2-(trifluoromethyl)benzoic acid)

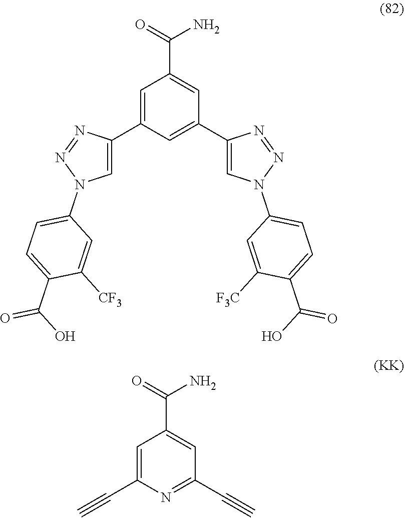 [SMILES: NC(=O)c1cc(-c2cn(-c3ccc(C(=O)O)c(C(F)(F)F)c3)nn2)cc(-c2cn(-c3ccc(C(=O)O)c(C(F)(F)F)c3)nn2)c1 ; C#Cc1cc(C(N)=O)cc(C#C)n1]

2,6-Diethynylpyridine-4-carboxamide KK was prepared according to Example 17 using the HATU coupling of 2,6-diethynylpyridine-4-carboxylic acid with ammonia. Compound 82 was made using the copper click method in Example 1 using diethynyl KK and azide U. Compound 82 Mass Spec (ESI Negative Mode): Calcd for C₂₆H₁₄F₆N₈O₅ 632.44; Found: 631.1 [M−H⁺]

### Example 39

### Synthesis of 4,4′-((4-(ethoxycarbonyl)pyridine-2,6-diyl)bis(1H-1,2,3-triazole-4,1-diyl))bis(2-(trifluoromethyl)benzoic acid)

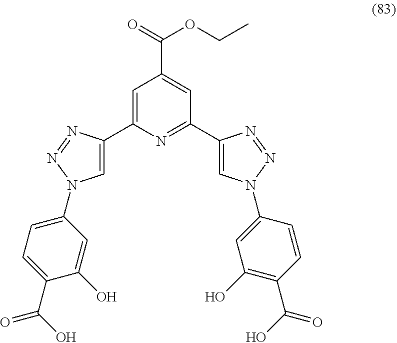 [SMILES: CCOC(=O)c1cc(-c2cn(-c3ccc(C(=O)O)c(O)c3)nn2)nc(-c2cn(-c3ccc(C(=O)O)c(O)c3)nn2)c1]

Compound 83 was made using the copper click method in Example 1 using ethyl-2,6-diethynylpyridine-4-carboxylate O and azide U. Compound 83 Mass Spec (ESI Negative Mode): Calcd for C₂₈H₁₇F₆N₇O₆ 661.48; Found: 659.9 [M−H⁺].

### Example 40

### Synthesis of 4,4′-((4-(azetidine-1-carbonyl)pyridine-2,6-diyl)bis(1H-1,2,3-triazole-4,1-diyl))bis(2-hydroxybenzoic acid)

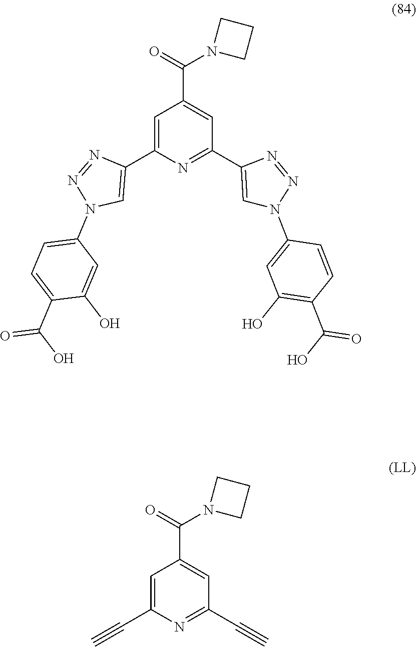 [SMILES: O=C(O)c1ccc(-n2cc(-c3cc(C(=O)N4CCC4)cc(-c4cn(-c5ccc(C(=O)O)c(O)c5)nn4)n3)nn2)cc1O ; C#Cc1cc(C(=O)N2CCC2)cc(C#C)n1]

N-Azetidinyl-2,6-diethynylpyridine-4-carboxamide LL was prepared according to Example 17 using the HATU coupling of 2,6-diethynylpyridine-4-carboxylic acid with azetidine. Compound 84 was made using the copper click method in Example 1 using diethynyl LL and azide B. Compound 84 Mass Spec (ESI Negative Mode): Calcd for C₂₇H₂₀N₈O₇ 568.51; Found: 567 [M−H⁺].

### Example 41

### Synthesis of 4,4′-((4-(ethyl(methyl)carbamoyl)pyridine-2,6-diyl)bis(1H-1,2,3-triazole-4,1-diyl))bis(2-hydroxybenzoic acid)

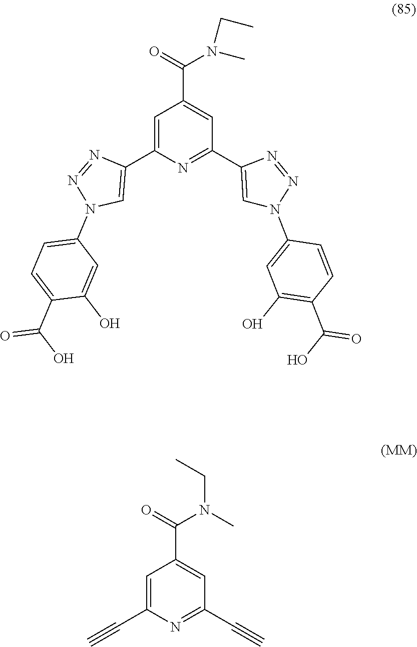 [SMILES: CCN(C)C(=O)c1cc(-c2cn(-c3ccc(C(=O)O)c(O)c3)nn2)nc(-c2cn(-c3ccc(C(=O)O)c(O)c3)nn2)c1 ; C#Cc1cc(C(=O)N(C)CC)cc(C#C)n1]

N-Methyl-N-ethyl-2,6-diethynylpyridine-4-carboxamide MM was prepared according to Example 17 using the HATU coupling of 2,6-diethynylpyridine-4-carboxylic acid with N-methyl-N-ethylamine. Compound 85 was made using the copper click method in Example 1 using diethynyl MM and azide B. Compound 85 Mass Spec (ESI Negative Mode): Calcd for C₂₇H₂₂N₈O₇ 570.52; Found: 569.1 [M−H⁺].

### Example 42

### Synthesis of n-ethyl-2,6-bis(1-(4-(2,2,2-trifluoroacetyl)phenyl)-1H-1,2,3-triazol-4-yl)isonicotinamide

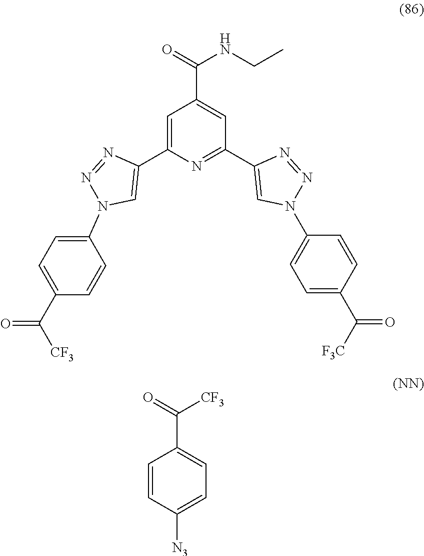 [SMILES: CCNC(=O)c1cc(-c2cn(-c3ccc(C(=O)C(F)(F)F)cc3)nn2)nc(-c2cn(-c3ccc(C(=O)C(F)(F)F)cc3)nn2)c1 ; [N-]=[N+]=Nc1ccc(C(=O)C(F)(F)F)cc1]

Diazotization of 1-(4-Aminophenyl)-2,2,2-trifluoroethan-1-one with sodium nitrite and sulfuric acid followed by displacement with azide (Org. Synth. 1942, 22, 96 DOI: 10.15227/orgsyn.022.0096) to form 1-(4-azidophenyl)-2,2,2-trifluoroethan-1-one NN which was purified via flash chromatography. Compound 86 was made using the copper click method in Example 1 using azide NN and diethynyl Q. Compound 86 Mass Spec (ESI Negative Mode): Calcd for C₂₈H₁₈F₆N₈O₃ 628.5; Found: 627 [M−H⁺].

### Example 43

### Synthesis of 4,4′-(pyridine-2,6-diylbis(1H-1,2,3-triazole-4,1-diyl))bis(2-(trifluoromethyl)benzoic acid)

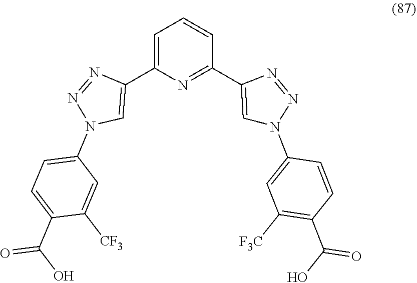 [SMILES: O=C(O)c1ccc(-n2cc(-c3cccc(-c4cn(-c5ccc(C(=O)O)c(C(F)(F)F)c5)nn4)n3)nn2)cc1C(F)(F)F]

Compound 87 was made using the copper click method in Example 1 using azide U and 2,6-diethynylpyridine A. Compound 87 Mass Spec (ESI Negative Mode): Calcd for C₂₅H₁₃F₆N₇O₄ 589.41; Found: 588.1 [M−H⁺].

### Example 44

### Synthesis of 4,4′-((4-(cyclopropylcarbamoyl)pyridine-2,6-diyl)bis(1H-1,2,3-triazole-4,1-diyl))bis(2-(trifluoromethyl)benzoic acid)

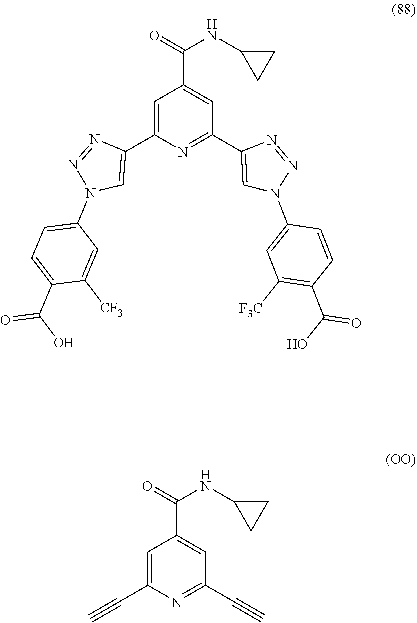

N-Cyclopropylamido-2,6-diethynylpyridine-4-carboxamide 00 was prepared according to Example 17 using the HATU coupling of 2,6-diethynylpyridine-4-carboxylic acid with cyclopropylamine. Compound 88 was made using the copper click method in Example 1 using diethynyl 00 and azide U. Compound 88 Mass Spec (ESI Negative Mode): Calcd for C₂₉H₁₈F₆N₈O₅ 672.50; Found: 671.1 [M−H⁺].

### Example 45

### Synthesis of 4,4′-((4-(butylcarbamoyl)pyridine-2,6-diyl)bis(1H-1,2,3-triazole-4,1-diyl))bis(2-(trifluoromethyl)benzoic acid)

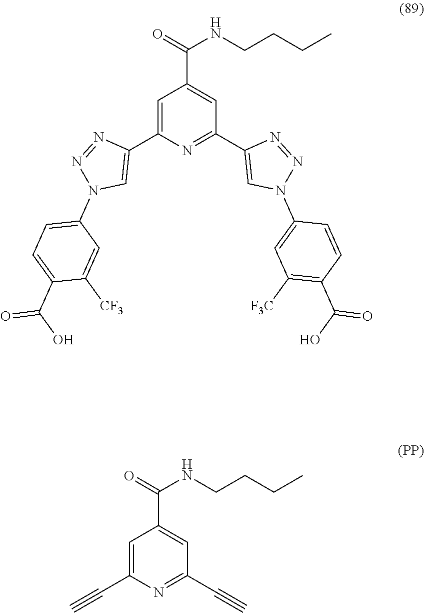 [SMILES: CCCCNC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1 ; C#Cc1cc(C(=O)NCCCC)cc(C#C)n1]

N-Butylamido-2,6-diethynylpyridine-4-carboxamide PP was prepared according to Example 17 using the HATU coupling of 2,6-diethynylpyridine-4-carboxylic acid with 1-aminobutane. Compound 89 was made using the copper click method in Example 1 using diethynyl PP and azide U. Compound 89 Mass Spec (ESI Negative Mode): Calcd for C₃₀H₂₂F₆N₈O₅ 688.55; Found: 687.1 [M−H⁺].

### Example 46

### Synthesis of 5,5′-((4-(diethylcarbamoyl)pyridine-2,6-diyl)bis(1H-1,2,3-triazole-4,1-diyl))bis(2-(trifluoromethyl)benzoic acid)

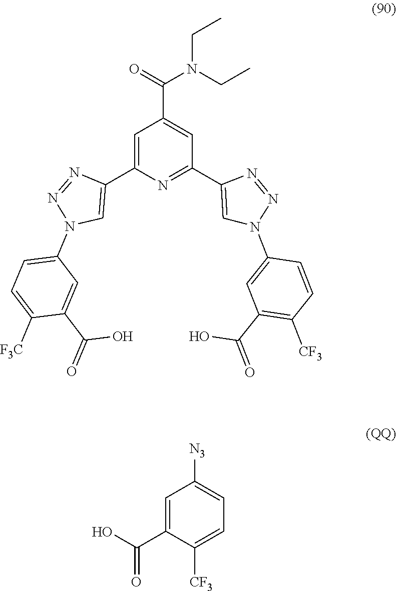 [SMILES: CCN(CC)C(=O)c1cc(-c2cn(-c3ccc(C(F)(F)F)c(C(=O)O)c3)nn2)nc(-c2cn(-c3ccc(C(F)(F)F)c(C(=O)O)c3)nn2)c1 ; [N-]=[N+]=Nc1ccc(C(F)(F)F)c(C(=O)O)c1]

Diazotization of 5-amino-2-(trifluoromethyl)benzoic acid was done according to Example 22 to make 5-azido-2-(trifluoromethyl)benzoic acid QQ. Compound 90 was made using the copper click method in Example 1 using azide QQ and diethynyl JJ. Compound 90 Mass Spec (ESI Negative Mode): Calcd for C₃₀H₂₂F₆N₈O₅ 688.55; Found: 687.1 [M−H⁺].

### Example 47

### Synthesis of 5,5′-((4-(morpholine-4-carbonyl)pyridine-2,6-diyl)bis(1H-1,2,3-triazole-4,1-diyl))bis(2-(trifluoromethyl)benzoic acid)

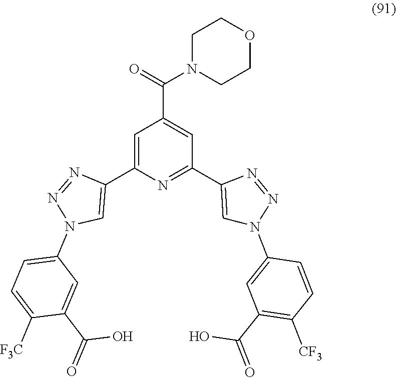 [SMILES: O=C(O)c1cc(-n2cc(-c3cc(C(=O)N4CCOCC4)cc(-c4cn(-c5ccc(C(F)(F)F)c(C(=O)O)c5)nn4)n3)nn2)ccc1C(F)(F)F]

Compound 91 was made using the copper click method in Example 1 using azide QQ and diethynyl I. Compound 91 Mass Spec (ESI Negative Mode): Calcd for C₃₀H₂₀F₆N₈O₆ 702.53; Found: 701 [M−H⁺].

### Example 48

### Synthesis of 4,4′-(pyridazine-3,6-diylbis(1H-1,2,3-triazole-4,1-diyl))bis(2-hydroxybenzoic acid)

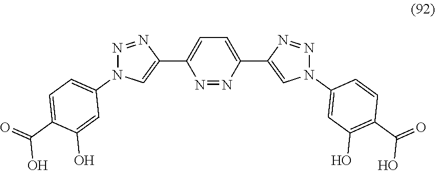 [SMILES: O=C(O)c1ccc(-n2cc(-c3ccc(-c4cn(-c5ccc(C(=O)O)c(O)c5)nn4)nn3)nn2)cc1O]

3,6-diethynylpyridazine was prepared according to the Sonogashira method described in Example 9 using 3,6-dibromopyridazine. Compound 92 was made using the copper click method in Example 1 using azide B and 3,6-diethynylpyridazine. Compound 92 Mass Spec (ESI Negative Mode): Calcd for C₂₂H₁₄N₈O₆ 486.1; Found: 485 [M−H⁺].

### Example 49

### Synthesis of 5,5′-((4-(ethylcarbamoyl)pyridine-2,6-diyl)bis(1H-1,2,3-triazole-4,1-diyl))bis(3-(trifluoromethyl)benzoic acid)

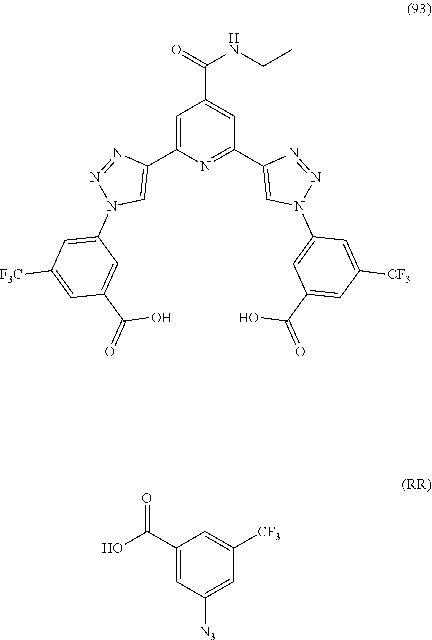 [SMILES: CCNC(=O)c1cc(-c2cn(-c3cc(C(=O)O)cc(C(F)(F)F)c3)nn2)nc(-c2cn(-c3cc(C(=O)O)cc(C(F)(F)F)c3)nn2)c1 ; [N-]=[N+]=Nc1cc(C(=O)O)cc(C(F)(F)F)c1]

Diazotization of 3-amino-5-(trifluoromethyl)benzoic acid was done according to Example 22 to make 3-azido-5-(trifluoromethyl)benzoic acid RR. Compound 93 was made using the copper click method in Example 1 using azide RR and diethynyl Q. Compound 93 Mass Spec (ESI Negative Mode): Calcd for C₂₈H₁₈F₆N₈O₅ 660.49; Found: 659 [M−H⁺].

### Example 50

### Synthesis of 4,4′-((4-carboxypyridine-2,6-diyl)bis(1H-1,2,3-triazole-4,1-diyl))bis(2-(trifluoromethyl)benzoic acid)

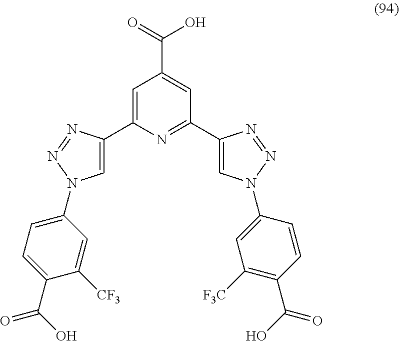 [SMILES: O=C(O)c1cc(-c2cn(-c3ccc(C(=O)O)c(C(F)(F)F)c3)nn2)nc(-c2cn(-c3ccc(C(=O)O)c(C(F)(F)F)c3)nn2)c1]

Compound 94 was made using the copper click method in Example 1 using azide U and 2,6-diethynylpyridine-4-carboxylic acid. Compound 94 Mass Spec (ESI Negative Mode): Calcd for C₂₆H₁₃F₆N₇O₆ 633.42; Found: 632 [M−H⁺].

### Example 51

### Synthesis of 3,3′-(((4-(ethylcarbamoyl)pyridine-2,6-diyl)bis(1H-1,2,3-triazole-4,1-diyl))bis(4,1-phenylene))dipropionic acid

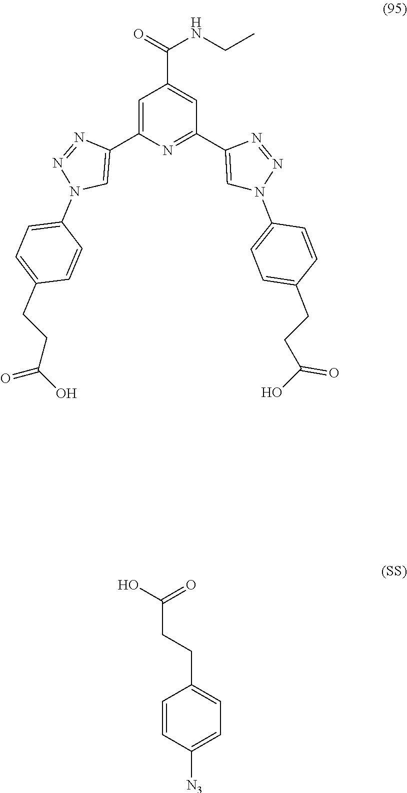 [SMILES: CCNC(=O)c1cc(-c2cn(-c3ccc(CCC(=O)O)cc3)nn2)nc(-c2cn(-c3ccc(CCC(=O)O)cc3)nn2)c1 ; [N-]=[N+]=Nc1ccc(CCC(=O)O)cc1]

Diazotization of 3-(4-aminophenyl)propanoic acid was done according to Example 22 to make 3-(4-azidophenyl)propanoic acid SS. Compound 95 was made using the copper click method in Example 1 using azide SS and diethynyl Q. Compound 95 Mass Spec (ESI Negative Mode): Calcd for C₃₀H₂₈N₈O₅ 580.61; Found: 579 [M−H⁺].

### Example 52

### Synthesis of 4,4′-(((4-(ethylcarbamoyl)pyridine-2,6-diyl)bis(1H-1,2,3-triazole-4,1-diyl))bis(4,1-phenylene))dibutyric acid

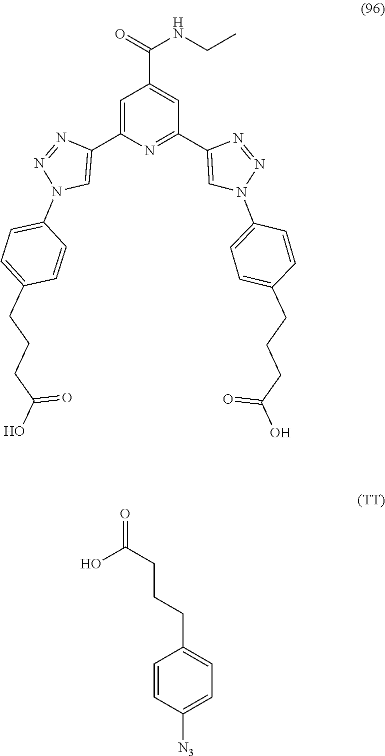 [SMILES: CCNC(=O)c1cc(-c2cn(-c3ccc(CCCC(=O)O)cc3)nn2)nc(-c2cn(-c3ccc(CCCC(=O)O)cc3)nn2)c1 ; [N-]=[N+]=Nc1ccc(CCCC(=O)O)cc1]

Diazotization of 4-(4-aminophenyl)butanoic acid was done according to Example 22 to make 4-(4-azidophenyl)butanoic acid TT. Compound 96 was made using the copper click method in Example 1 using azide TT and diethynyl Q. Compound 96 Mass Spec (ESI Negative Mode): Calcd for C₃₂H₃₂N₈O₅ 608.66; Found: 607 [M−H⁺].

### Example 53

### Synthesis of 4,4′-((4-(ethylcarbamoyl)pyridine-2,6-diyl)bis(1H-1,2,3-triazole-4,1-diyl))diphthalic acid

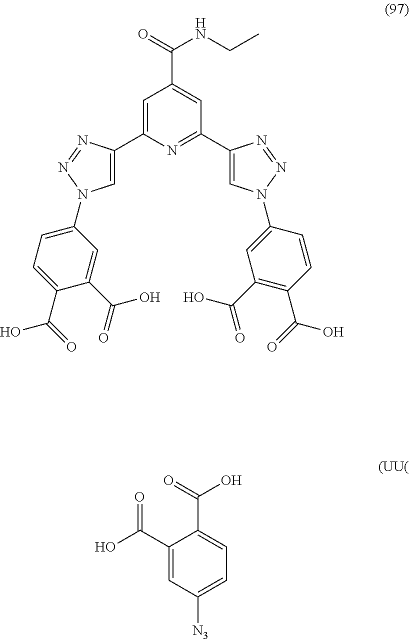 [SMILES: CCNC(=O)c1cc(-c2cn(-c3ccc(C(=O)O)c(C(=O)O)c3)nn2)nc(-c2cn(-c3ccc(C(=O)O)c(C(=O)O)c3)nn2)c1 ; [N-]=[N+]=Nc1ccc(C(=O)O)c(C(=O)O)c1]

Diazotization of 4-aminophthalic acid was done according to Example 22 to make 4-azidophthalic acid UU. Compound 97 was made using the copper click method in Example 1 using azide UU and diethynyl Q. Compound 97 Mass Spec (ESINegative Mode): Calcd for C₂₈H₂₀N₈O₉ 612.52; Found: 611.1 [M−H⁺].

### Example 54

### Synthesis of 4,4′-((4-(ethylcarbamoyl)pyridine-2,6-diyl)bis(1H-1,2,3-triazole-4,1-diyl))bis(2-methoxybenzoic acid)

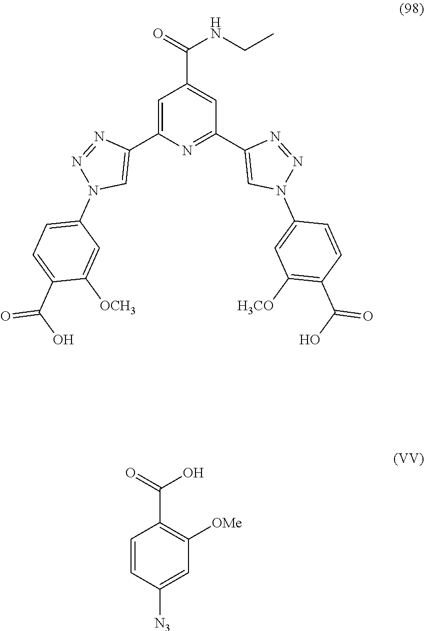 [SMILES: CCNC(=O)c1cc(-c2cn(-c3ccc(C(=O)O)c(OC)c3)nn2)nc(-c2cn(-c3ccc(C(=O)O)c(OC)c3)nn2)c1 ; COc1cc(N=[N+]=[N-])ccc1C(=O)O]

Diazotization of 4-amino-2-methoxybenzoic acid was done according to Example 22 to make 4-azido-2-methoxybenzoic acid VV. Compound 98 was made using the copper click method in Example 1 using azide VV and diethynyl Q. Compound 98 Mass Spec (ESI Negative Mode): Calcd for C₂₈H₂₄N₈O₇ 584.55; Found: 583 [M−H⁺].

### Example 55

### Synthesis of 5,5′-((4-(ethylcarbamoyl)pyridine-2,6-diyl)bis(1H-1,2,3-triazole-4,1-diyl))diisophthalic acid

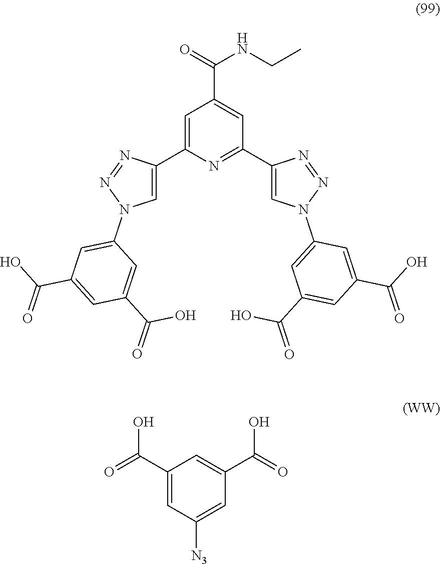 [SMILES: CCNC(=O)c1cc(-c2cn(-c3cc(C(=O)O)cc(C(=O)O)c3)nn2)nc(-c2cn(-c3cc(C(=O)O)cc(C(=O)O)c3)nn2)c1 ; [N-]=[N+]=Nc1cc(C(=O)O)cc(C(=O)O)c1]

Diazotization of 4-aminoisophthalic acid was done according to Example 22 to make 4-azidoisophthalic acid WW. Compound 99 was made using the copper click method in Example 1 using azide WW and diethynyl Q. Compound 99 Mass Spec (ESI Negative Mode): Calcd for C₂₈H₂₀N₈O₉ 612.52; Found: 611 [M−H⁺].

### Example 56

### Synthesis of 4,4′-((4-(ethylcarbamoyl)pyridine-2,6-diyl)bis(1H-1,2,3-triazole-4,1-diyl))bis(3-hydroxybenzoic acid)

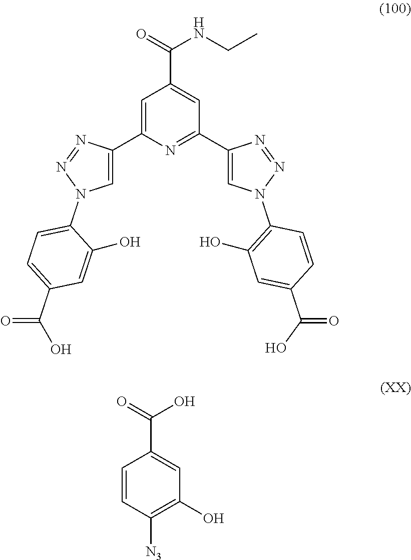 [SMILES: CCNC(=O)c1cc(-c2cn(-c3ccc(C(=O)O)cc3O)nn2)nc(-c2cn(-c3ccc(C(=O)O)cc3O)nn2)c1 ; [N-]=[N+]=Nc1ccc(C(=O)O)cc1O]

Diazotization of 4-amino-3-hydroxybenzoic acid was done according to Example 22 to make 4-azido-3-hydroxybenzoic acid XX. Compound 100 was made using the copper click method in Example 1 using azide XX and diethynyl Q. Compound 100 Mass Spec (ESI Negative Mode): Calcd for C₂₆H₂₀N₈O₇ 556.50; Found: 555.1 [M−H⁺].

### Example 57

### Synthesis of diethyl (3-(4-(6-(1-(3-(diethoxyphosphoryl)propyl)-1H-1,2,3-triazol-4-yl)-4-(ethylcarbamoyl) 303yridine-2-yl)-1H-1,2,3-triazol-1-yl)propyl) phosphonate

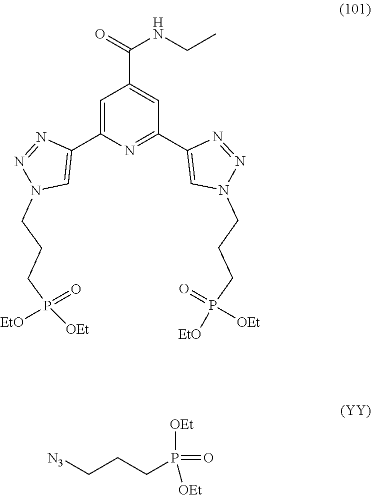 [SMILES: CCNC(=O)c1cc(-c2cn(CCCP(=O)(OCC)OCC)nn2)nc(-c2cn(CCCP(=O)(OCC)OCC)nn2)c1 ; CCOP(=O)(CCCN=[N+]=[N-])OCC]

Diazotization of 0,0-diethyl (3-aminopropyl)phophonate was done according to Example 22 to make 0,0-diethyl (3-azidopropyl)phophonate YY. Compound 101 was made using the copper click method in Example 1 using azide YY and diethynyl Q. Compound 101 Mass Spec (ESI Negative Mode): Calcd for C₂₆H₄₂N₈O₇P₂ 640.62; Found: 639.3 [M−H⁺].

### Example 58

### Synthesis of 4,4′-((4-(ethylcarbamoyl)pyridine-2,6-diyl)bis(1H-1,2,3-triazole-4,1-diyl))bis(2-methylbenzoic acid)

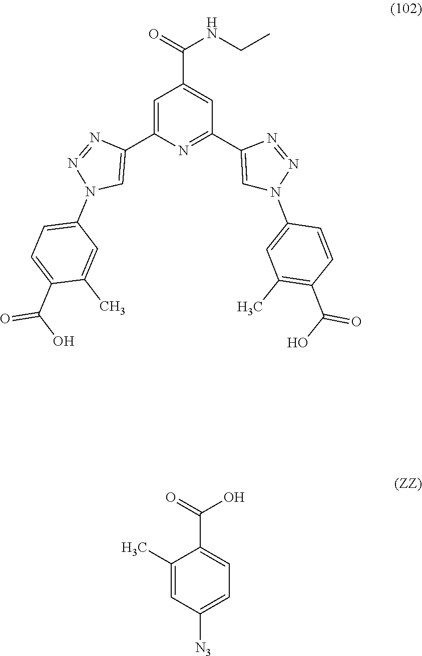 [SMILES: CCNC(=O)c1cc(-c2cn(-c3ccc(C(=O)O)c(C)c3)nn2)nc(-c2cn(-c3ccc(C(=O)O)c(C)c3)nn2)c1 ; Cc1cc(N=[N+]=[N-])ccc1C(=O)O]

Diazotization of 4-amino-2-methylbenzoic acid was done according to Example 22 to make 4-amino-2-methylbenzoic acid ZZ. Compound 102 was made using the copper click method in Example 1 using azide ZZ and diethynyl Q. Compound 102 Mass Spec (ESI Negative Mode): Calcd for C₂₈H₂₄N₈O₅ 552.55; Found: 551.1 [M−H⁺].

### Example 59

### Synthesis of 4,4′-((5-carboxy-1,3-phenylene)bis(1H-1,2,3-triazole-4,1-diyl))bis(2-(trifluoromethyl)benzoic acid)

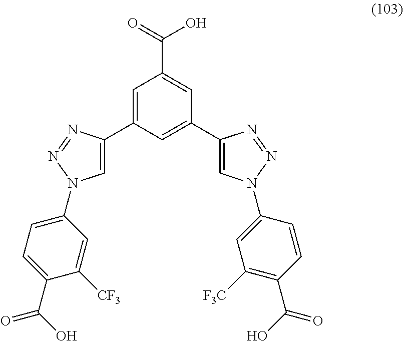 [SMILES: O=C(O)c1cc(-c2cn(-c3ccc(C(=O)O)c(C(F)(F)F)c3)nn2)cc(-c2cn(-c3ccc(C(=O)O)c(C(F)(F)F)c3)nn2)c1]

Compound 103 was made using the copper click method in Example 1 using azide Q and 3,5-diethynylbenzoic acid. Compound 103 Mass Spec (ESI Negative Mode): Calcd for C₂₇H₁₄F₆N₆O₆ 632.44; Found: 631 [M−H⁺].

### Example 60

### Synthesis of 2,2′-((4-(ethylcarbamoyl)pyridine-2,6-diyl)bis(1H-1,2,3-triazole-4,1-diyl))bis(3-(trifluoromethyl)benzoic acid)

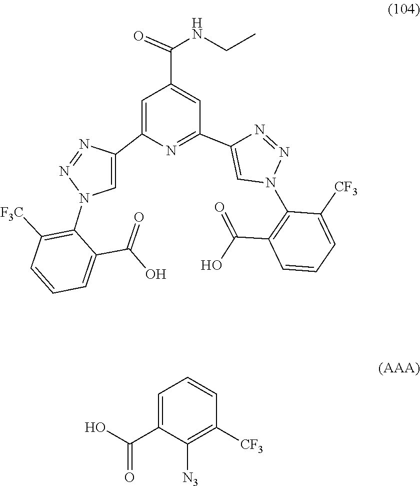 [SMILES: CCNC(=O)c1cc(-c2cn(-c3c(C(=O)O)cccc3C(F)(F)F)nn2)nc(-c2cn(-c3c(C(=O)O)cccc3C(F)(F)F)nn2)c1 ; [N-]=[N+]=Nc1c(C(=O)O)cccc1C(F)(F)F]

Diazotization of 2-amino-3-(trifluoromethyl)benzoic acid was done according to Example 22 to make 2-azido-3-(trifluoromethyl)benzoic acid AAA. Compound 104 was made using the copper click method in Example 1 using azide AAA and diethynyl Q. Compound 104 Mass Spec (ESI Negative Mode): Calcd for C₂₈H₁₈F₆N₈O₅ 660.49; Found: 659 [M−H⁺].

### Example 61

### Synthesis of 4,4′-((4-((2-hydroxyethyl)carbamoyl)pyridine-2,6-diyl)bis(1H-1,2,3-triazole-4,1-diyl))bis(2-(trifluoromethyl)benzoic acid)

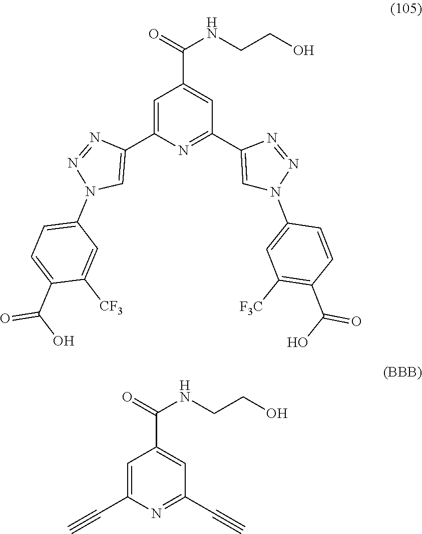 [SMILES: O=C(NCCO)c1cc(-c2cn(-c3ccc(C(=O)O)c(C(F)(F)F)c3)nn2)nc(-c2cn(-c3ccc(C(=O)O)c(C(F)(F)F)c3)nn2)c1 ; C#Cc1cc(C(=O)NCCO)cc(C#C)n1]

N-(2-Hydroxyethyl)amino-2,6-diethynylpyridine-4-carboxamide BBB was prepared according to Example 17 using the HATU coupling of 2,6-diethynylpyridine-4-carboxylic acid with aminoethanol. Compound 105 was made using the copper click method in Example 1 using diethynyl BBB and azide U. Compound 105 Mass Spec (ESI Negative Mode): Calcd for C₂₈H₁₈F₆N₈O₆ 676.49; Found: 675 [M−H⁺].

### Example 62

### Synthesis of 4,4′-((4-(ethylcarbamoyl)pyridine-2,6-diyl)bis(1H-1,2,3-triazole-4,1-diyl))bis(2-nitrobenzoic acid)

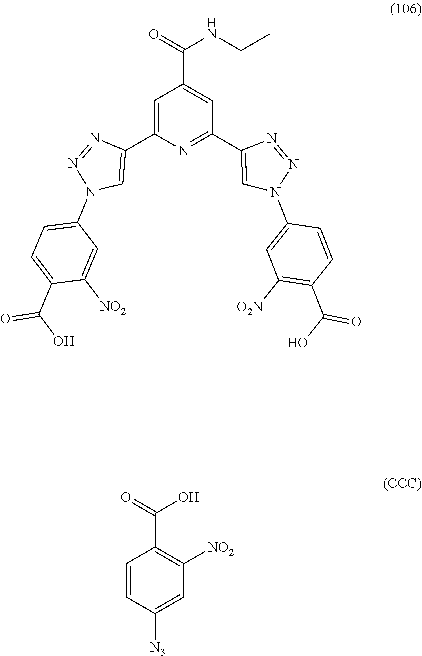 [SMILES: CCNC(=O)c1cc(-c2cn(-c3ccc(C(=O)O)c([N+](=O)[O-])c3)nn2)nc(-c2cn(-c3ccc(C(=O)O)c([N+](=O)[O-])c3)nn2)c1 ; [N-]=[N+]=Nc1ccc(C(=O)O)c([N+](=O)[O-])c1]

Diazotization of 4-amino-2-nitrobenzoic acid was done according to Example 22 to make 4-azido-2-nitrobenzoic acid CCC. Compound 106 was made using the copper click method in Example 1 using azide CCC and diethynyl Q. Compound 106 Mass Spec (ESI Negative Mode): Calcd for C₂₆H₁₈N₁₀O₉ 614.49; Found: 613 [M−H⁺].

### Example 63

### Synthesis of 4,4′-((4-((3,3,3-trifluoropropyl)carbamoyl)pyridine-2,6-diyl)bis(1H-1,2,3-triazole-4,1-diyl))bis(2-(trifluoromethyl)benzoic acid)

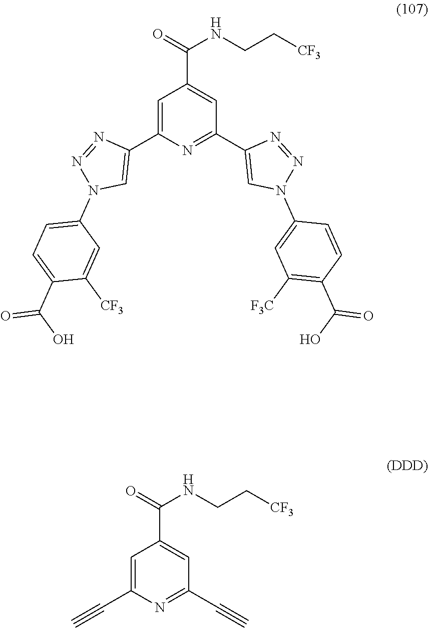 [SMILES: O=C(NCCC(F)(F)F)c1cc(-c2cn(-c3ccc(C(=O)O)c(C(F)(F)F)c3)nn2)nc(-c2cn(-c3ccc(C(=O)O)c(C(F)(F)F)c3)nn2)c1 ; C#Cc1cc(C(=O)NCCC(F)(F)F)cc(C#C)n1]

N-(3,3,3-trifluoropropyl)-2,6-diethynylpyridine-4-carboxamide DDD was prepared according to Example 17 using the HATU coupling of 2,6-diethynylpyridine-4-carboxylic acid with 3,3,3-trifluoropropan-1-amine. Compound 107 was made using the copper click method in Example 1 using diethynyl DDD and azide U. Compound 107 Mass Spec (ESI Negative Mode): Calcd for C₂₉H₁₇F₉N₈O₅ 728.49; Found: 727 [M−H⁺].

### Example 64

### Synthesis of 4,4′,4″,4′″-((((butane-1,4-diylbis(azanediyl))bis(carbonyl))bis(pyridine-4,2,6-triyl))tetrakis(1H-1,2,3-triazole-4,1-diyl))tetrakis(2-(trifluoromethyl)benzoic acid)

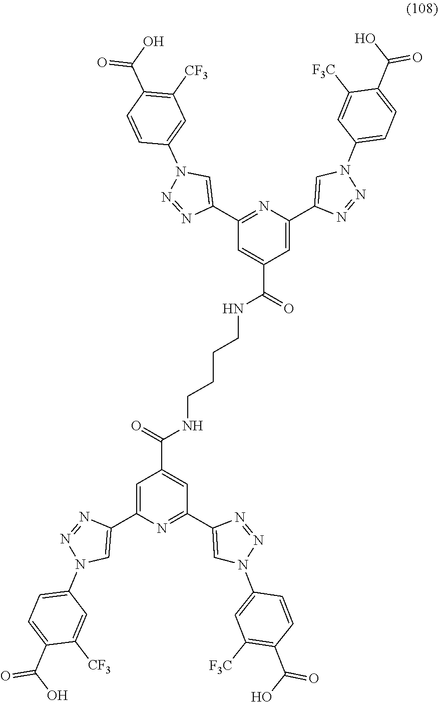 [SMILES: O=C(NCCCCNC(=O)c1cc(-c2cn(-c3ccc(C(=O)O)c(C(F)(F)F)c3)nn2)nc(-c2cn(-c3ccc(C(=O)O)c(C(F)(F)F)c3)nn2)c1)c1cc(-c2cn(-c3ccc(C(=O)O)c(C(F)(F)F)c3)nn2)nc(-c2cn(-c3ccc(C(=O)O)c(C(F)(F)F)c3)nn2)c1]

Compound 108 was made using the copper click method in Example 1 using diethynyl FF and azide U. Compound 108 Mass Spec (ESI Negative Mode): Calcd for C₅₆H₃₄F₁₂N₁₆O₁₀ 1318.97; Found: 1316.9 [M−H⁺].

### Example 65

### Synthesis of (4-(4-(4-(ethylcarbamoyl)-6-(1-(4-phosphonophenyl)-1H-1,2,3-triazol-4-yl)309yridine-2-yl)-1h-1,2,3-triazol-1-yl)phenyl)phosphonic acid

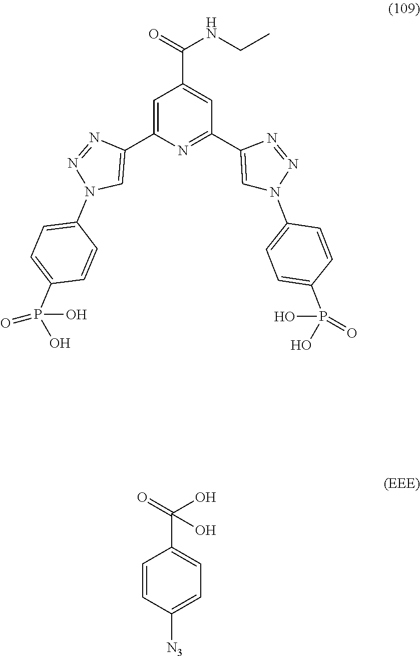

Diazotization of (4-aminophenyl)phosphonic acid was done according to Example 22 to make (4-azidophenyl)phosphonic acid EEE. Compound 109 was made using the copper click method in Example 1 using azide EEE and diethynyl Q. Compound 109 Mass Spec (ESI Negative Mode): Calcd for C₂₄H₂₂N₈O₇P₂ 596.44; Found: 595 [M−H⁺].

### Example 66

### Synthesis of 2,2′-((4,4′-((4-(ethylcarbamoyl)pyridine-2,6-diyl)bis(1H-1,2,3-triazole-4,1-diyl))bis(2-(trifluoromethyl)benzoyl))bis(azanediyl))diacetic acid

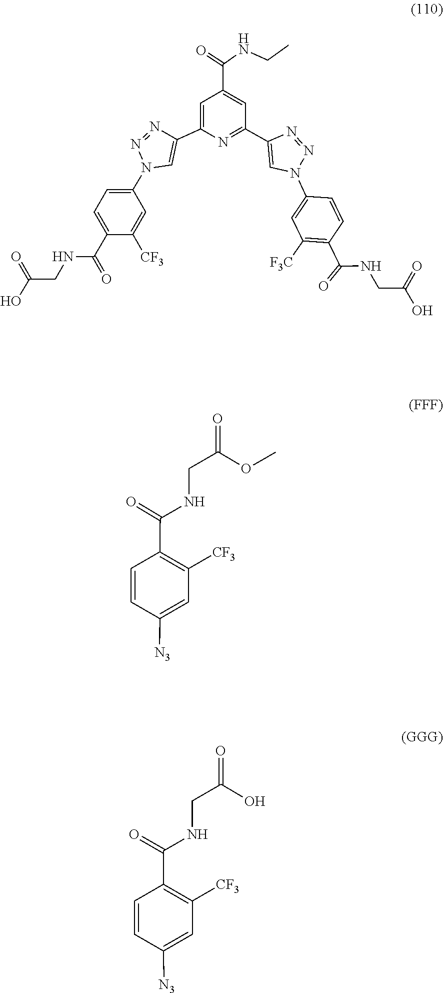 [SMILES: CCNC(=O)c1cc(-c2cn(-c3ccc(C(=O)NCC(=O)O)c(C(F)(F)F)c3)nn2)nc(-c2cn(-c3ccc(C(=O)NCC(=O)O)c(C(F)(F)F)c3)nn2)c1 ; COC(=O)CNC(=O)c1ccc(N=[N+]=[N-])cc1C(F)(F)F ; [N-]=[N+]=Nc1ccc(C(=O)NCC(=O)O)c(C(F)(F)F)c1]

Methyl (4-Azido-2-(trifluoromethyl)benzoyl)glycine FFF was prepared according to the HATU condensation method described in Example 17 coupling of azide U with methyl glycinate. The methyl ester FFF was saponified with 1N NaOH followed by neutralization to give (4-Azido-2-(trifluoromethyl)benzoyl)glycine GGG. Compound 110 was made using the copper click method in Example 1 using diethynyl Q and azide GGG. Compound 110 Mass Spec (ESI Negative Mode): Calcd for C₃₂H₂₄F₆N₁₀O₇ 774.60; Found: 773 [M−H⁺].

### Example 67

### Synthesis of dimethyl 2,2′-((4,4′-((4-(ethylcarbamoyl)pyridine-2,6-diyl)bis(1H-1,2,3-triazole-4,1-diyl))bis(2-(trifluoromethyl)benzoyl))bis(azanediyl))diacetate

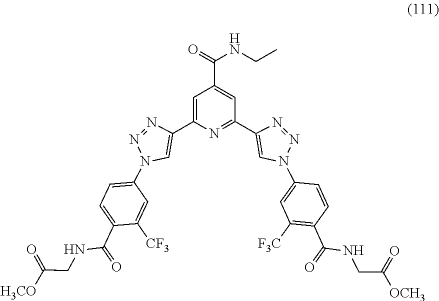 [SMILES: CCNC(=O)c1cc(-c2cn(-c3ccc(C(=O)NCC(=O)OC)c(C(F)(F)F)c3)nn2)nc(-c2cn(-c3ccc(C(=O)NCC(=O)OC)c(C(F)(F)F)c3)nn2)c1]

Compound 111 was made using the copper click method in Example 1 using azide FFF and diethynyl Q. Compound 111 Mass Spec (ESI Negative Mode): Calcd for C₃₄H₂₈F₆N₁₀O₇ 802.65; Found: 801.1 [M−H⁺].

### Example 68

### Synthesis of (2S,2′S)-2,2′-((4,4′-((4-(ethylcarbamoyl)pyridine-2,6-diyl)bis(1H-1,2,3-triazole-4,1-diyl))bis(2-(trifluoromethyl)benzoyl))bis(azanediyl))disuccinic acid

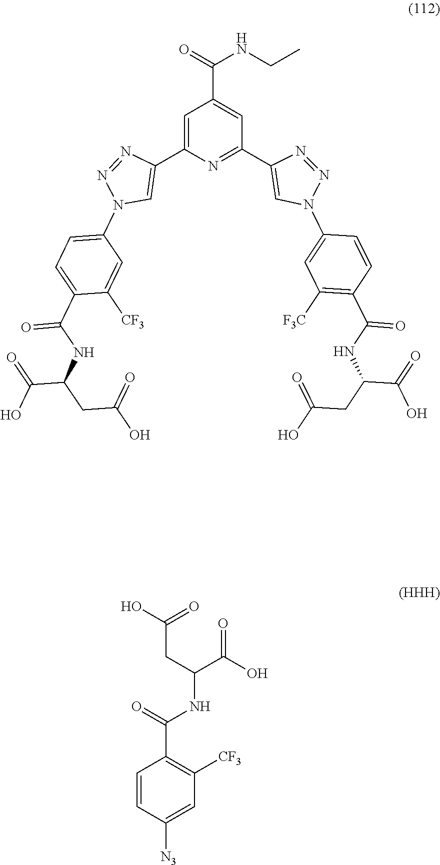 [SMILES: CCNC(=O)c1cc(-c2cn(-c3ccc(C(=O)N[C@@H](CC(=O)O)C(=O)O)c(C(F)(F)F)c3)nn2)nc(-c2cn(-c3ccc(C(=O)N[C@@H](CC(=O)O)C(=O)O)c(C(F)(F)F)c3)nn2)c1 ; [N-]=[N+]=Nc1ccc(C(=O)NC(CC(=O)O)C(=O)O)c(C(F)(F)F)c1]

(4-azido-2-(trifluoromethyl)benzoyl)aspartate HHH was prepared according to the HATU condensation method described in Example 17 coupling of azide U with dimethyl aspartate to give dimethyl (4-azido-2-(trifluoromethyl)benzoyl)aspartate. The dimethyl ester of was saponified with 1N NaOH followed by neutralization to give HHH. Compound 112 was made using the copper click method in Example 1 using diethynyl Q and azide HHH. Compound 112 Mass Spec (ESI Negative Mode): Calcd for C₃₆H₂₈F₆N₁₀O₁₁ 890.67; Found: 889 [M−H⁺].

### Example 69

### Synthesis of 2,2′-((2,2′-((4,4′-((4-(ethylcarbamoyl)pyridine-2,6-diyl)bis(1H-1,2,3-triazole-4,1-diyl))bis(2-(trifluoromethyl)benzoyl))bis(azanediyl))bis(acetyl))bis(azanediyl))diacetic acid

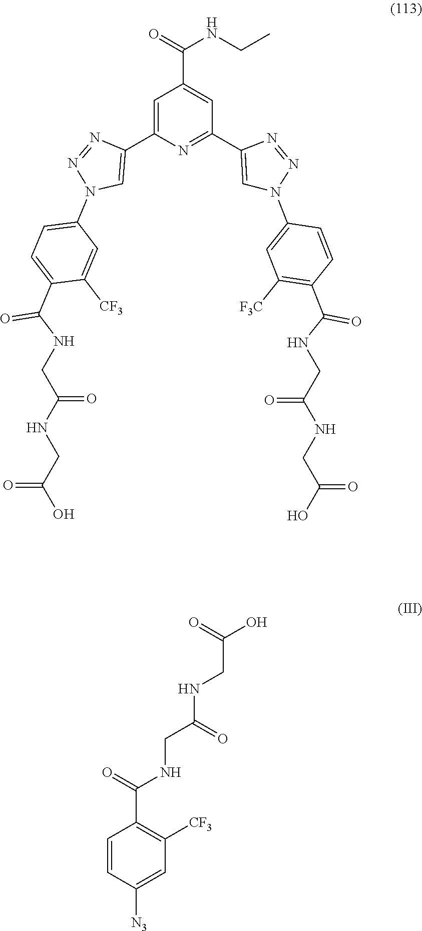 [SMILES: CCNC(=O)c1cc(-c2cn(-c3ccc(C(=O)NCC(=O)NCC(=O)O)c(C(F)(F)F)c3)nn2)nc(-c2cn(-c3ccc(C(=O)NCC(=O)NCC(=O)O)c(C(F)(F)F)c3)nn2)c1 ; [N-]=[N+]=Nc1ccc(C(=O)NCC(=O)NCC(=O)O)c(C(F)(F)F)c1]

(4-azido-2-(trifluoromethyl)benzoyl)glycylglycine III was prepared according to the HATU condensation method described in Example 17 coupling azide U with methyl-glycylglycinate. The methyl ester was saponified with 1N NaOH followed by neutralization to give (4-azido-2-(trifluoromethyl)benzoyl)-glycylglycine III. Compound 113 was made using the copper click method in Example 1 using diethynyl Q and azide III. Compound 113 Mass Spec (ESI Negative Mode): Calcd for C₃₆H₃₀F₆N₁₂O₉ 888.70; Found: 887 [M−H⁺].

### Example 70

### Synthesis of 2,6-bis(1-(4-cyano-3-(trifluoromethyl)phenyl)-1H-1,2,3-triazol-4-yl)-n-ethylisonicotinamide

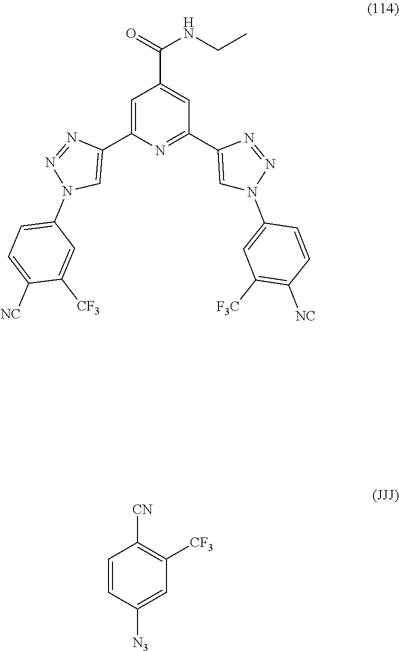

Diazotization of 4-amino-2-(trifluoromethyl)benzonitrile was done according to Example 22 to make 4-azido-2-(trifluoromethyl)benzonitrile JJJ. Compound 114 was made using the copper click method in Example 1 using azide JJJ and diethynyl Q. Compound 114 Mass Spec (ESI Negative Mode): Calcd for C₂₈H₁₆F₆N₁₀O 622.50; Found: 621 [M−H⁺].

### Example 71

### Synthesis of 4,4′-((5-carboxy-1,3-phenylene)bis(1H-1,2,3-triazole-1,4-diyl))bis(2-(trifluoromethyl)benzoic acid)

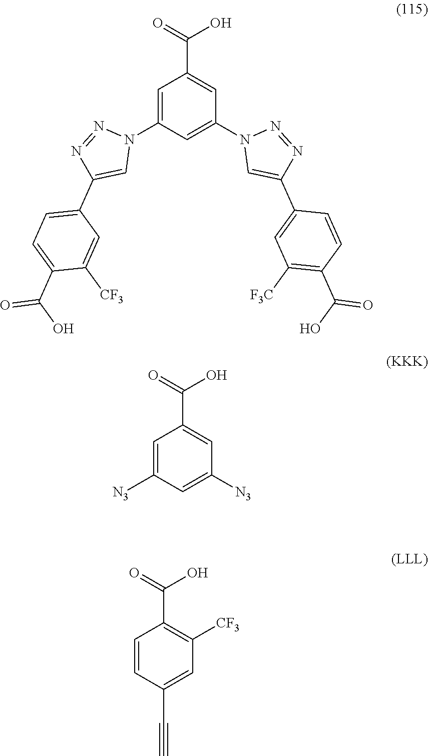 [SMILES: O=C(O)c1cc(-n2cc(-c3ccc(C(=O)O)c(C(F)(F)F)c3)nn2)cc(-n2cc(-c3ccc(C(=O)O)c(C(F)(F)F)c3)nn2)c1 ; [N-]=[N+]=Nc1cc(N=[N+]=[N-])cc(C(=O)O)c1 ; C#Cc1ccc(C(=O)O)c(C(F)(F)F)c1]

Diazotization of 3,5-diaminobenzoic acid was done according to Example 22 to make 3,5-diazidobenzoic acid KKK. 4-Ethynyl-2-(trifluoromethyl)benzoic acid LLL was prepared according to the Sonogashira method described in Example 9 using 4-bromo-2-(trifluoromethyl)benzoic acid. Compound 115 was made using the copper click method in Example 1 using bis-azide KKK and mono-ethynyl LLL. Compound 115 Mass Spec (ESI Negative Mode): Calcd for C₂₇H₁₄F₆N₆O₆ 632.44; Found: 631 [M−H⁺].

### Example 72

### Synthesis of 4,4′-((5-(ethylcarbamoyl)-1,3-phenylene)bis(1H-1,2,3-triazole-1,4-diyl))bis(2-(trifluoromethyl)benzoic acid)

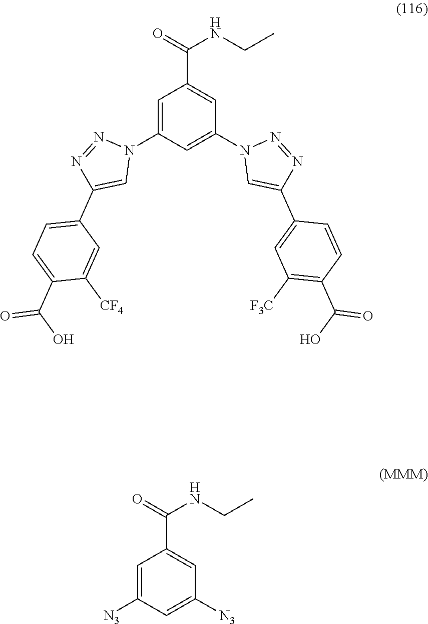

3,5-Diazido-N-ethylbenzamide MMM was prepared according to the HATU condensation method described in Example 17 coupling Bis-azide acid KKK with ethylamine. Compound 116 was made using the copper click method in Example 1 using mono-ethynyl LLL and bis-azide MMM. Compound 116 Mass Spec (ESI Negative Mode): Calcd for C₂₉H₁₉F₆N₇O₅ 659.51; Found: 658.1 [M−H⁺].

### Example 73

### Synthesis of 4,4′-(thiophene-2,5-diylbis(1H-1,2,3-triazole-4,1-diyl))bis(2-(trifluoromethyl)benzoic acid)

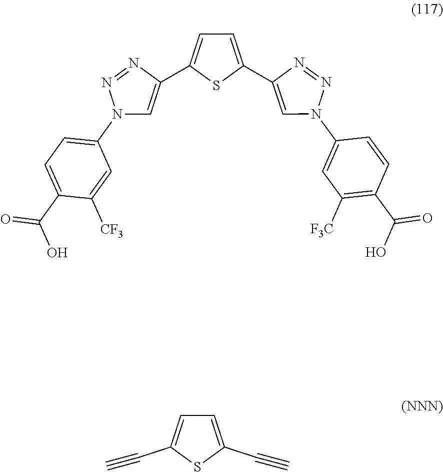

2,5-Diethynylthiophene NNN was prepared according to the Sonogashira method described in Example 9 using 2,5-dibromothiophene. Compound 117 was made using the copper click method in Example 1 using azide U and diethynyl NNN. Compound 117 Mass Spec (ESI Negative Mode): Calcd for C₂₄H₁₂F₆N₆O₄ 594.45; Found: 593 [M−H⁺].

### Example 74

### Synthesis of 4,4′-(furan-2,5-diylbis(1H-1,2,3-triazole-4,1-diyl))bis(2-(trifluoromethyl)benzoic acid)

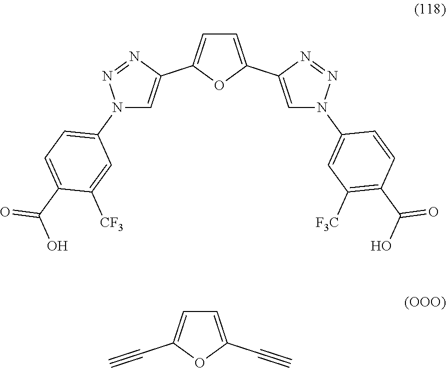

2,5-Diethynylfuran OOO was prepared according to the Sonogashira method described in Example 9 using 2,5-dibromofuran. Compound 118 was made using the copper click method in Example 1 using azide U and diethynyl 000. Compound 118 Mass Spec (ESI Negative Mode): Calcd for C₂₄H₁₂F₆N₆O₅ 578.39; Found: 577 [M−H⁺].

### Example 75

### Synthesis of 3′-(4-(4-(ethylcarbamoyl)-6-(1-(3′-(trifluoromethyl)-[1,1′-biphenyl]-3-yl)-1H-1,2,3-triazol-4-yl)pyridin-2-yl)-1H-1,2,3-triazol-1-yl)-3-(trifluoromethyl)-[1,1′-biphenyl]-4-carboxylic acid-carbon dioxide (1/1)

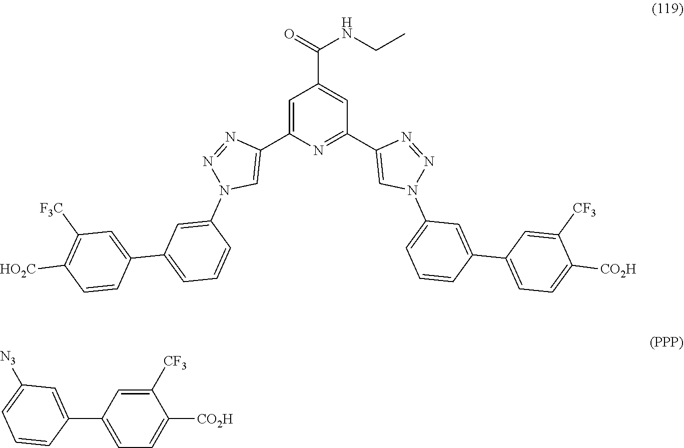

Synthesis of 3′-amino-3-(trifluoromethyl)-[1,1′-biphenyl]-4-carboxylic acid commenced with mixing methyl 4-bromo-2-(trifluoromethyl)benzoate (142 mg, 0.5 mmol), 3-aminophenyl boronic acid (137 mg, 1 mmol), Pd(PPh₃)₂Cl₂ (35 mg, 0.1 mmol) and K₂CO₃(138 mg, 2 mmol) was added methanol (2 ml). The solution was heated at 45C for 1.5 hr. The methanol was removed by evaporation. The residual solid was dissolved in dimethylformamide (1 ml) and purified by silica chromatography using a Reveleris Prep eluting with a EtOAc/hexane mobile phase. Appropriate fractions were collected based on UV and evaporated to give methyl 3′-amino-3-(trifluoromethyl)-[1,1′-biphenyl]-4-carboxylate (101 mg, 68% yield).

Diazotization of methyl 3′-amino-3-(trifluoromethyl)-[1,1′-biphenyl]-4-carboxylate was done according to Example 22 to make methyl 3′-azido-3-(trifluoromethyl)-[1,1′-biphenyl]-4-carboxylate. The methyl ester was saponified KOH, neutralized with 0.25 HCl and recovered with EtOAc to give 3′-azido-3-(trifluoromethyl)-[1,1′-biphenyl]-4-carboxylic acid PPP. Compound 119 was made using the copper click method in Example 1 using azide PPP and diethynyl Q. Compound 119 Mass Spec (ESI Negative Mode): Calcd for C₄₀H₂₆N₈O₅ 812.69.

### Example 76

### Synthesis of 4,4′-((4-(ethylcarbamoyl)pyridine-2,6-diyl)bis(1H-1,2,3-triazole-4,1-diyl))bis(2-cyanobenzoic acid)

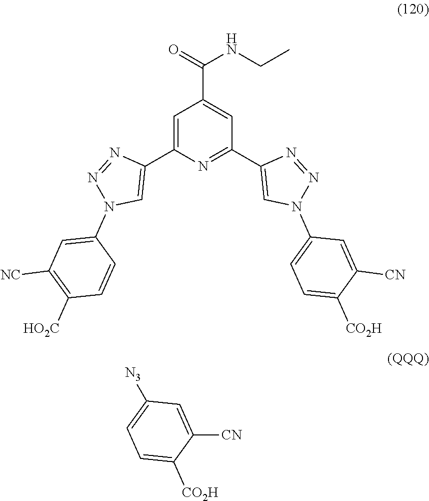

Diazotization of 4-amino-2-cyanobenzoic acid was done according to Example 22 to make 4-azido-2-cyanobenzoic acid QQQ. Compound 120 was made using the copper click method in Example 1 using azide QQQ and diethynyl Q. Compound 120 Mass Spec (ESI Negative Mode): Calcd for C₂₈H₁₈N₁₀O₅ 574.50.

### Example 77

### Synthesis of 4,4′-((4-(ethylcarbamoyl)pyridine-2,6-diyl)bis(1H-1,2,3-triazole-4,1-diyl))bis(2-chlorobenzoic acid)

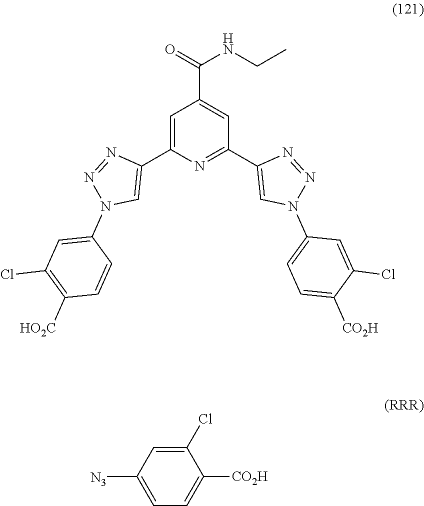

Diazotization of 4-amino-2-chlorobenzoic acid was done according to Example 22 to make 4-azido-2-chlorobenzoic acid RRR. Compound 121 was made using the copper click method in Example 1 using azide RRR and diethynyl Q. Compound 121 Mass Spec (ESI Negative Mode): Calcd for C₂₆H₁₈Cl₂N₈O₅ 593.38.

### Example 78

### Screen of PEMs for Enhancement of XNTP Polymerization

The Sequencing by Expansion (SBX) methodology developed by the inventors provides significant performance enhancements in sequence read efficiency and accuracy of Xpandomers relative to native DNA. However, initial transcription of the sequence of the natural DNA template onto the measurable Xpandomer relies on the ability of DNA polymerase to utilize XNTPs as substrates (the generalized structure of an XNTP is discussed herein with reference to FIG. 1A and FIG. 2). The inventors have found that most DNA polymerases do not efficiently polymerize XNTPs. In an effort to improve the efficiency and accuracy of XNTP polymerization into Xpandomers, several PEMs were screened for the ability to enhance DNA polymerase primer extension reactions using XNTPs as substrates.

A representative primer extension reaction may include the following reagents: 2 pmol primer, 2.2 pmol 45mer oligonucleotide template, 50 pmol of each XNTP (XATP, XCTP, XGTP, and XTTP), 50 Mm Tris HCl, Ph 6.79, 200 Mm NaCl, 20% PEG, 5% NMS, 0.5 nmol polyphosphate 60.19, 0.3 Mm MnCl2, and 0.6 μg of purified recombinant DNA polymerase protein. Reactions may be run for 1 hour at 23 °C. Reaction products (i.e., constrained Xpandomers) are treated to cleave the phosphoramidate bonds, thereby generating linearized Xpandomers. Reaction products may be analyzed using gel electrophoresis on 4-12% acrylamide gels to resolve and visualize Xpandomer products of different lengths. For the PEM screen described above, PEMs were typically tested in the micro to millimolar range.

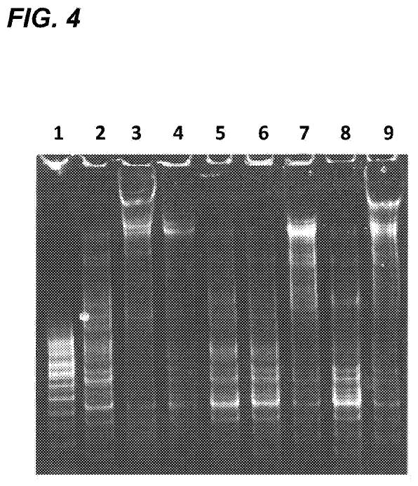

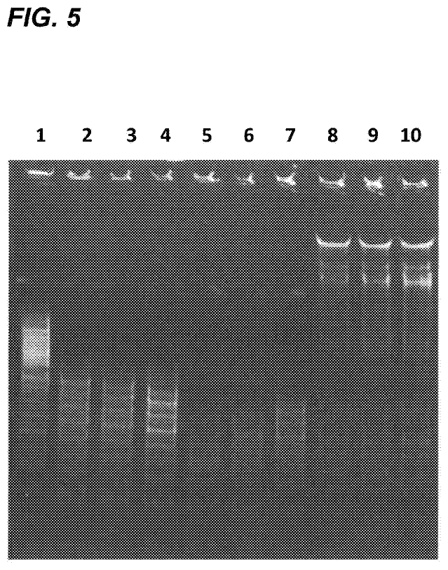

Surprisingly, several PEMs were observed to significantly and reproducibly enhance DNA polymerase-mediated primer extension with XNTPs. Representative gels demonstrating this enhancement are presented in FIGS. 4 and 5. With reference to FIG. 4, as can be seen in lane 1 (no PEM additive), DNA polymerase extends the template bound primer with up to only around 14 XNTPs under these conditions. However, addition certain PEMs to the primer extension reaction enables the polymerase to synthesize considerably longer extension products as can be seen, e.g., in lanes 3 (compound 4) 7 (compound 3) and 9 (compound 1). In contrast, several different aromatic compounds had little or no effect on XNTP polymerization (see, e.g., lanes 2, 4-6, and 8), indicating that PEM activity is specific for compounds 1, 3, and 4.

Similarly, with reference to FIG. 5, in the absence of PEM additive, DNA polymerase shows modest primer extension activity with XNTPs (lane 1, no PEM additive), while addition of compound 2 at various concentrations (lanes 8-10) significantly enhances primer extension activity. Again, this PEM activity is specific for compound 2, as other unrelated aromatic compounds had no effect (lanes 2-7).

### Example 79

### PEMs Enhance Sequencing by Expansion (SBX)

To investigate the accuracy of PEM-dependent enhancement of XNTP polymerization, primer extension products were sequenced using the SBX protocol. Briefly, the constrained Xpandomer products of XNTP polymerization are cleaved to generate linearized Xpandomers. This is accomplished by first quenching the extension reaction with a solution containing 100 Mm EDTA, 2 Mm THPTA, and 2% Tween-20. Then the sample is subjected to amine modification with a solution of 1 M NaHCO₃ and 1 M succinic anhydride in DMF. Cleavage of the phosphoramidate bonds is carried out with 37% HCl and linearized Xpandomers are purified with QIAquick columns (QIAGEN, Inc.).

For sequencing, protein nanopores are prepared by inserting a-hemolysin into a DphPE/hexadecane bilayer member in buffer B1, containing 2 M NH₄Cl and 100 Mm HEPES, Ph 7.4. The cis well is perfused with buffer B2, containing 0.4 M NH₄Cl, 0.6 M GuCl, and 100 Mm HEPES, Ph 7.4. The Xpandomer sample is heated to 70 °C for 2 minutes, cooled completely, then a 2 μL sample is added to the cis well. A voltage pulse of 90Mv/390Mv/10 μs is then applied and data is acquired via Labview acquisition software.

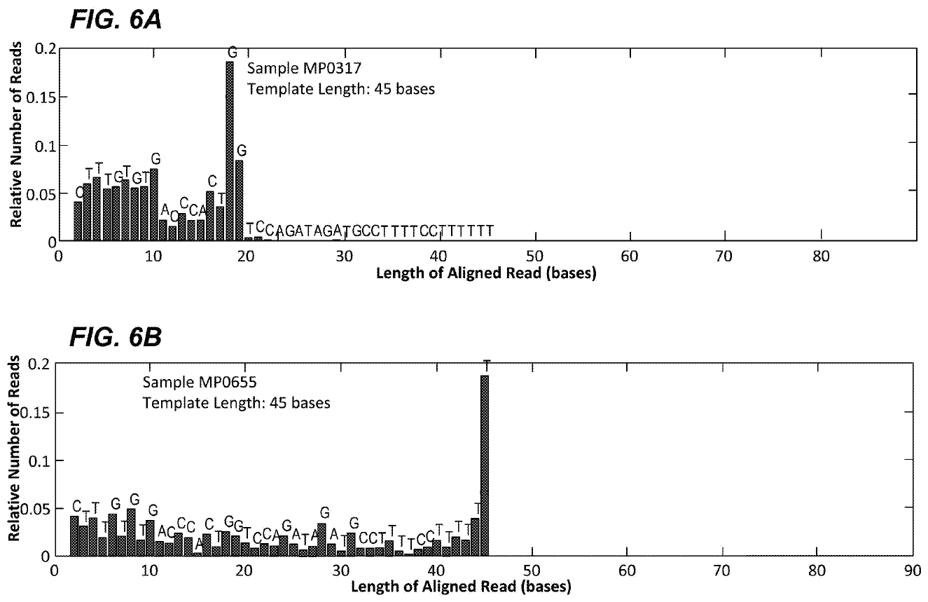

Sequence data is analyzed by histogram display of the population of sequence reads from a single SBX reaction. The analysis software aligns each sequence read to the sequence of the template and trims the extent of the sequence at the end of the reads that does not align with the correct template sequence. Representative histograms of SBX sequencing of a 45mer template are presented in FIG. 6A (no additive control) and FIG. 6B (SBX in the presence of PEM compound 1). As can be seen, in the absence of compound 1, sequence reads are not accurate past around base 18 of the template. Notably, addition of compound 1 to the SBX reaction increased the accuracy of the sequence reads across the entire length of the 45mer template.

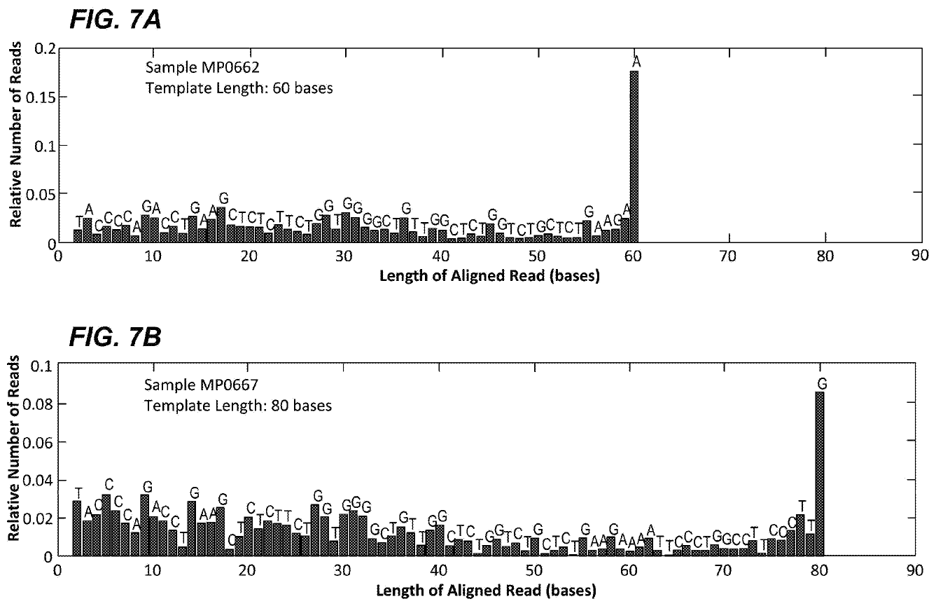

These results inspired additional experiments to test the ability of PEM compound 1 to enhance SBX of even longer templates. FIGS. 7A and 7B show histograms of SBX sequencing of 60mer and 80mer templates, respectively. Surprisingly, compound 1 enabled accurate sequence reads completely to the end of each of these longer templates. These results demonstrate robust and accurate enhancement of XNTP polymerization activity by a novel PEM that powerfully increases the capability of SBX to provide nanopore-based nucleic acid sequence information.

### Example 80

### PEMs Enable Synthesis of Long Xpandomer Products

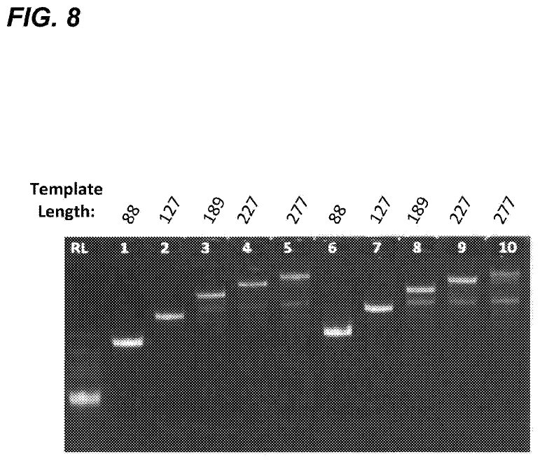

Following the success of accurately replicating templates of up to 80 nucleotides in length into Xpandomers, XNTP polymerization reactions were conducted using four longer templates, consisting of 88, 127, 227, and 277 nucleotides in length. A variant of DP04 DNA polymerase, referred to as C4552 (SEQ ID NO:1), was used in these polymerization reactions and reaction conditions were optimized for C4552 activity in the presence of PEM compound 1. Other suitable DP04 polymerase variants include, but are not limited to, those of SEQ ID NOS: 2-5. In addition to 1Mm compound 1, reaction additives included 1Mm urea and 2.75 μg single-strand binding protein (Eco SSB). Extension reactions were carried out with 0.85 pmol template, 0.5 pmol oligonucleotide primer, and 1 nmol each XNTP in a final volume of 10 μL. Reactions were run in buffer composed of 50Mm TrisCl, Ph 8.84, 200Mm NH₄Oac, and 20% PEG8K supplemented with 5% NMS, polyphosphate PP-60.20 in amounts of 3 or 4 nmol, and 2Mm MnCl₂. 1.2 μg purified recombinant DNA polymerase protein was used in each extension reaction and reactions were run for 1-2 hours at 23 °C. Results of representative extension reactions using the longer templates are shown in FIG. 8. Notably, in the presence of compound 1, the polymerase was able polymerize XNTPs to generate complete Xpandomer copies of each longer template, ranging from 88 (lanes 1 and 6) to 277 (lanes 5 and 10) nucleotides in length. Lanes 1-5 and 6-10 represent identical extension reactions with the exception of the amount of PP-60.20 additive, which was 3nmol in lanes 1-5 and 4nmol in lanes 6-10. These results underscore the surprising advantages conferred by compound 1 in reactions requiring polymerization of non-natural, highly substituted nucleotide analogs by DNA polymerase and suggest that this compound, as well as other PEMs, could greatly expand the potential of the SBX sequencing protocol.

### Example 81

### Next Generation PEMs Enhance Polymerization of XNTPS to Generate Long Xpandomer Products

Based on the advantageous properties observed with PEM compound 1, a next generation of PEM compounds was designed with the objective of improving certain properties, including, but not limited to, water solubility of the molecules. Representative next generation PEM structures are described in Examples 9-34 and Table 1.

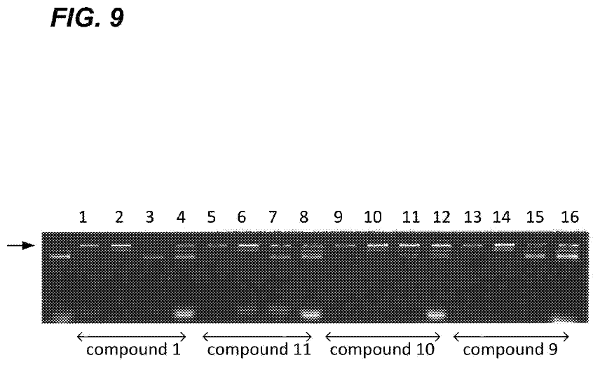

PEM activity of compounds 9-11 was tested in primer extension assays using three 100mer templates, derived from the HIV1, 2, and 3 genomes. Primer extension reactions included the following reagents: 75 Mm TrisCl, Ph 8.44, 175 Mm NH₄₀ac, 20% PEG8K, 5% NMS, 0.8 nmol PP-60.20, 0.6 Mm MnCl₂, 2.3 μg Tth single-strand binding protein (SSB), 0.5 M or 1 M urea, 200 pmol each XNTP, 1.1 pmol template, 1 pmol oligonucleotide primer, 1.2 μg purified recombinant C4552 DNA polymerase, and 0.5 Mm PEM. 10 μL primer extension reactions were run for 30 minutes at 23 °C and reaction products were analyzed by gel electrophoresis. A representative gel showing primer extension products is presented in FIG. 9. As shown in lanes 1 (HIV1 template), 2 (HIV2 template), and 4 (HIV3 template, no SSB and 1 M urea), compound 1 enables polymerization of XNTPs into full length Xpandomer copies of the three different 100mer templates (gel migration position of the 100mer indicated by the arrow). Likewise, each of compounds 9 (lanes 13-16), 10 (lanes 9-12), and 11 (lanes 5-8) enable XNTP polymerization at least as efficiently as compound 1 on each of the three different 100mer templates. These results suggest that PEM activity may be optimized by increasing various physicochemical properties of the compounds, such as water solubility.

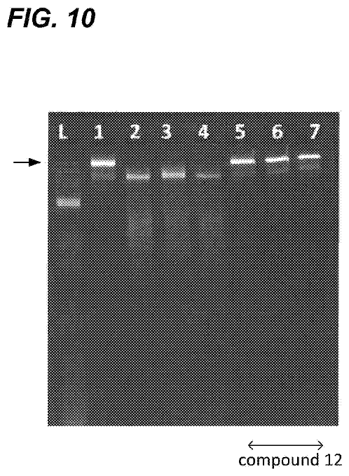

PEM activity of compound 12 was tested in primer extension assays using the HIV2 100mer template. Primer extension reactions included the following reagents: 50 Mm TrisCl, 200 Mm NH₄Oac, 20% PEG8K, 5% NMS, 0.6 nmol PP-60.20, 0.6 Mm MnCl₂, 2.75 μg/μl Eco single-strand binding protein (SSB), 1 M urea, 50 pmol each XNTP, 1.1 pmol template, 1 pmol oligonucleotide primer, 1.2 μg/μl purified recombinant C4760 DNA polymerase (SEQ ID NO: 2), and 0.5, 1, or 1.5 Mm PEM. 10 μL primer extension reactions were run for 30 minutes at 23 °C and reaction products were analyzed by gel electrophoresis. A representative gel showing primer extension products is presented in FIG. 10. As shown in lanes 5 (0.5 Mm PEM), 6 (1 Mm PEM), and 7 (1.5 Mm PEM), compound 12 enables polymerization of XNTPs into full length Xpandomer copies of the 100mer template (gel migration position of the 100mer indicated by the arrow) in a manner comparable to that of compound 10 (lane 1). Lanes 2-4 show primer extension products from reactions with a structurally related additive lacking robust PEM activity. These results suggest that PEM activity may be determined by very specific chemical structures.

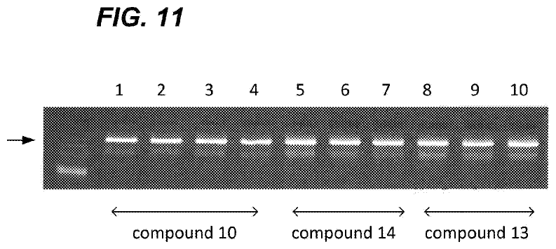

PEM activity of compounds 13 and 14 was tested in primer extension assays using the HIV2 100mer template. Primer extension reactions included the following reagents: 50 Mm TrisCl, 200 Mm NH₄Oac, 20% PEG8K, 5% NMS, 0.6 nmol PP-60.20, 0.6 Mm MnCl₂, 2.75 μg/μl Eco single-strand binding protein (SSB), 1 M urea, 50 pmol each XNTP, 1.1 pmol template, 1 pmol oligonucleotide primer, 1.2 μg/μl purified recombinant C4760 DNA polymerase (a variant of DPO4, see SEQ ID NO: 2), and 0.5, 1, 1.52, or 2.5 Mm PEM. 10 μL primer extension reactions were run for 30 minutes at 23 °C and reaction products were analyzed by gel electrophoresis. A representative gel showing primer extension products is presented in FIG. 11 (the position of the full length HIV2 100mer is indicated by the arrow). As shown in lanes 5-7 (compound 14 at various concentrations) and 8-10 (compound 13 at various concentrations), each of these next generation PEMs enables polymerization of XNTPs into full length Xpandomer copies of the 100mer template in a manner comparable to that of compound 10 (lanes 1-4).

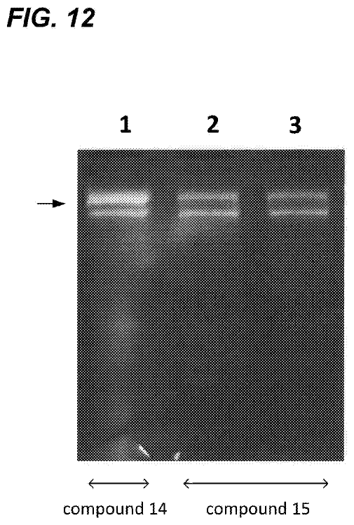

PEM activity of compound 15 was tested in primer extension assays using a 411mer amplicon template. Primer extension reactions included the following reagents: 50 Mm TrisCl, 200 Mm NH₄₀ac, 20% PEG8K, 5% NMS, 3 nmol PP-60.20, 2 Mm MnCl₂, 2 μg Kod single-strand binding protein (SSB), 1 M urea, 250 pmol each XNTP, 1 pmol template, 1 pmol oligonucleotide primer, 1.2 μg purified recombinant C4760 DNA polymerase (a variant of DPO4, see SEQ ID NO: 2), and 2 (lane 2) or 3 (lane 3) Mm PEM. 10 μL primer extension reactions were run for 20 minutes at 37 °C and reaction products were analyzed by gel electrophoresis. A representative gel showing primer extension products is presented in FIG. 12 (the position of a 277mer is indicated by the arrow). As shown in lanes 2 and 3 (compound 15 at two different concentrations) this next generation PEM enables polymerization of XNTPs into lengthy Xpandomer copies of the 411mer template in a manner comparable to that of compound 14 (lane 1). Remarkably, the polymerase is completely dependent upon the addition of PEM to the reaction in order to be capable of synthesizing these lengthy Xpandomer products. Even longer extension products may be obtained by optimizing various reaction parameters, e.g., extension time and/or concentrations of various additives.

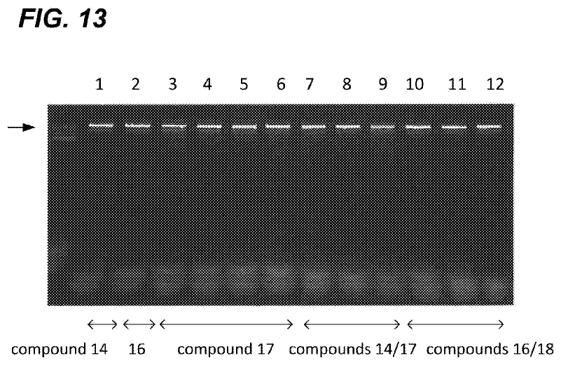

PEM activity of compounds 16, 17, and 18 and combinations thereof were tested in primer extension assays using the HIV2-derived 100mer template. Primer extension reactions included the following reagents: 50 Mm TrisCl, 200 Mm NH₄Oac, 20% or 25% PEG8K, 5% NMS, 0.6 nmol PP-60.20, 0.6 Mm MnCl₂, 2 μg Kod single-strand binding protein (SSB), 1 M urea, 50 pmol each XNTP, 1 pmol template, 1 pmol oligonucleotide primer, 1.2 μg purified recombinant C4760 DNA polymerase (a variant of DPO4, see SEQ ID NO: 2), and 0.5-2 Mm PEM. 10 μL primer extension reactions were run for 30 minutes at 37 °C and reaction products were analyzed by gel electrophoresis. A representative gel showing primer extension products is presented in FIG. 13 (the position of a 100mer product is indicated by the arrow). As shown in lanes 2 (2 Mm compound 16) and 3-6 (0.5, 1-, 2-, and 3-Mm compound 17) these next generation PEMs enable polymerization of XNTPs into lengthy Xpandomer copies of the 100mer template in a manner comparable to that of compound 14 (lane 1). In addition, combinations of 2 Mm compound 14 and 0.1 Mm (lane 7), or 0.3 Mm (lanes 8 and 9) compound 17 also enabled polymerization of XNTPs into lengthy Xpandomer copies of the 100mer template, indicating that combinations of PEMs may permit use of lower doses of each individual PEM. Similarly, combinations of 2 Mm compound 16 and 0.1 Mm (lane 10), or 0.3 Mm (lanes 11 and 12) compound 18 also appeared to permit use of lower doses of each individual PEM to enable polymerization of XNTPS into full length copies of the 100mer template.

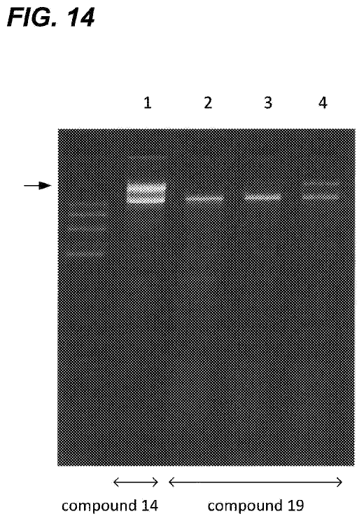

PEM activity of compound 19, was tested in primer extension assays using the 411mer amplicon template. Primer extension reactions included the following reagents: 50 Mm TrisCl, 200 Mm NH₄Oac, 20% PEG8K, 5% NMS, 3 nmol PP-60.20, 2 Mm MnCl₂, 2 μg Kod single-strand binding protein (SSB), 1 M urea, 250 pmol each XNTP, 0.5 pmol template,0.5 pmol oligonucleotide primer, 1.2 μg purified recombinant C4760 DNA polymerase (a variant of DPO4, see SEQ ID NO: 2), and 0.5 (lane 2), 1 (lane 3), or 1.5 Mm (lane 4) PEM. 10 μL primer extension reactions were run for 30 minutes at 37 °C and reaction products were analyzed by gel electrophoresis. A representative gel showing primer extension products is presented in FIG. 14 (the position of a 277mer product is indicated by the arrow). As shown in lanes 2-4 this next generation PEM enables polymerization of XNTPs into lengthy Xpandomer copies of the 411mer template, albeit in a manner less efficient than that of compound 14 (lane 1). These results suggest that PEM activity may be specific to the structure of the PEM and/or the length of the template.

### Example 82

### PEM Compounds

To explore the relationship between PEM structure and polymerase-enhancing activities, certain PEM compounds (see Table 1, herein) were synthesized (see synthetic examples herein) and tested to determine whether the PEM compounds (or compositions including the PEM compounds) enhance nucleic acid polymerization activity (i.e., whether they had a “PEM effect,” e.g., enhancing a nucleic acid polymerase reaction, increasing processivity, etc.). In some instances, the PEM compounds of the present disclosure were tested in primer extension reactions using DNA templates of the following lengths: A) 45mer; B) 100mer; C) 150mer; and D) 222mer. Primer extension reactions were run under conditions comparable to those described in Examples 77 and 78 and extension products were similarly analyzed by gel electrophoresis. A preliminary functional characterization of compounds displaying PEM activity is summarized in Table 2 below.

TABLE 2
PEM Activity Summary

| Functional Category | PEM compounds |
|---|---|
| PEM effect | 1-14, 16-27, 29-71, 75-76, 78-111, |
| | 113, 116-121, 176, 190-192, 206, 213- |
| | 214, 224, 233-234, 236, 240-241, 245- |
| | 247 |
| AA (replication of | 95, 96, 97, 108, 110, 111, 114, 116 |
| 45 mer template) | |
| B (replication of | 81, 82, 83, 84, 85, 86, 87, 88, 89, 90, 91, |
| 100 mer template) | 92, 93, 94, 106, 107, 112, 113, 115, 117, |
| | 118 |
| C (replication of | 80 |
| 150 mer template) | |
| D (replication of | 73, 79, 105, 120, 121 |
| 222 mer template) | |

### Example 83

### PEMs Enable Synthesis of Xpandomer Copies of an RNA Template

To begin to investigate the potential of PEMs to enhance diverse polymerase reactions, the ability of PEMS to enable DNA polymerase to utilize RNA as a template was tested. An initial screen of several compounds set forth in Table 1 was conducted using a 45mer RNA template derived from the HIV2 genome sequence. Primer extension reactions included the following reagents: 50 Mm TrisCl, 200 Mm NH₄OAc, 20% PEG8K, 5% NMS, 0.6 nmol PP-60.20, 0.6 Mm MnCl₂, 2 μg Kod single-strand binding protein (SSB), 1 M urea, 50 pmol each XNTP, 0.5 pmol template, 0.5 pmol oligonucleotide primer, 1.2 μg purified recombinant C4760 DNA polymerase (a variant of DPO4, see SEQ ID NO: 2), and 2 Mm PEM. 10 μL primer extension reactions were run for 30 minutes at 37 °C and reaction products were analyzed by gel electrophoresis. Surprisingly, several compounds were found to display PEM activity on an RNA template; these RNA PEMs include the following compounds: compound 51, 73, 49, 17, 75, 76, 93, 105, 106, 110, 116, and 118. These results suggest that certain PEMs may find utility in enabling Sequencing by Expansion of RNA templates.

All of the U.S. patents, U.S. patent application publications, U.S. patent applications, foreign patents, foreign patent applications and non-patent publications referred to in this specification and/or listed in the Application Data Sheet, including but not limited to, U.S. Provisional Patent Application No. 62/867,049 filed on Jun. 26, 2019, are incorporated herein by reference, in their entirety. Such documents may be incorporated by reference for the purpose of describing and disclosing, for example, materials and methodologies described in the publications, which might be used in connection with the presently described disclosure. The publications discussed above and throughout the text are provided solely for their disclosure prior to the filing date of the present application. Nothing herein is to be construed as an admission that the inventors are not entitled to antedate any referenced publication by virtue of prior disclosure.

### Example 84

### PEMs Enable Synthesis of Long Xpandomers

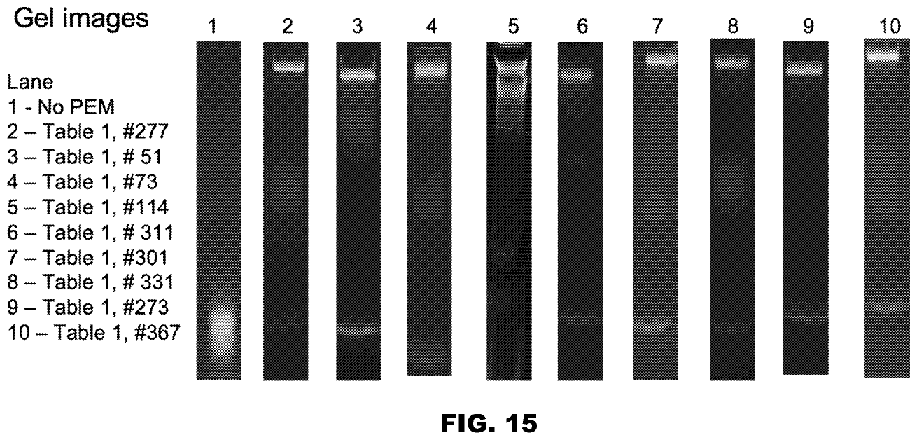

To further determine the ability of PEMs to enhance Xpandomer synthesis, a series of tests were performed using a 370-nt template. Primer extension reactions included the following reagents: 50 mM TrisCl, 150 mM NaCl, 250 mM Imidazole, 20% PEG8K, 8% Acetamide, 3% 1,2 Propane diol, 1 nmol polyphosphate, 0.5 mM MnCl₂, 0.5 μg Tth single-strand binding protein (SSB), 250 pmol each XNTP, 0.3 pmol template, 0.4 pmol oligonucleotide primer, 0.7 μg purified recombinant C7326 DNA polymerase (a variant of DPO4, see SEQ ID NO: 2), and 15 mM PEM. 10 μL primer extension reactions were run for 20 minutes at 37 °C, then 100 minutes at 42 °C, and reaction products were analyzed by gel electrophoresis. The identity of the PEM was varied, spanning several core and arm structures, and compared to a reaction without a PEM. Gels for several of these reactions are shown in FIG. 15 and include the following compounds: compound 277, 51, 73, 114, 311, 301, 331, 273, and 367 (according to the number of Table 1). Several structural elements differed between these compounds, including the ring size and composition of the core, as well as the identity and conjugation of the arms. These results suggest that certain PEMs could enabling Sequencing by Expansion of longer templates.

What is claimed is:

1. A compound having Formula (I): 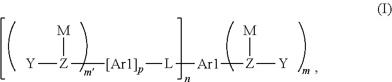
or a solvate, hydrate, tautomer, chelate or salt thereof,
wherein
a is 0 or an integer ranging from 1-4;
a′ is 0 or an integer ranging from 1-4;
m is 1, 2 or 3;
m′ is 1, 2 or 3;
n is 0, 1 or 2;
p is 0, 1 or 2;
Z is a 5-membered or 6-membered heteroaromatic ring including one, two, three, or four heteroatoms selected from O, N, or S;
Y is, at each occurrence, independently selected from Ar2, —C(O)—Ar2, —(CH₂)ₐAr2, —(CH₂)₃PO(OEt)₂, or —CH₂CO₂Me;
L is a linking group;
M is, at each occurrence, independently selected from hydrogen, halogen and C₁-C₄ alkyl;
Ar1 is, at each occurrence, independently selected from optionally substituted phenyl, pyridine, bipyridine, tripyridine, pyrazine, pyridazine, furan, dibenzofuran, thiophene, pyrrole, selenophene, naphthalene, fluorene, phenanthrene, cinnoline, phthalazine, quinazoline, quinoxaline, naphthyridine, phenanthroline, purine, and carbazole,
wherein substituents for Ar1 are, at each occurrence, independently selected from halogen, —OH, —CN, —NO₂, C₁-C₆ alkyl, C₁-C₆ haloalkyl, C₁-C₆ heteroalkyl, C₁-C₆ cycloalkyl, —OR⁰, —CONH₂, —C(O)NR¹R^{1′}, —C(O)(CH₂)ₐNR¹R^{1′}, —NR¹R^{1′}, —NR¹C(O)R³, —C(O)SR³, —COR³, —CO(CH₂)ₐOC(O)R³, —OC(O)R³, —C(O)OR³, —C—O—R³, mercaptan, —R⁴—H, —SOR¹, —S(O)₂R¹, —S(O)₂NR¹R^{1′}, —CH₂—NR¹S(O)₂R³, —NR¹S(O)₂R³, and —C(CH₃)═N-(phenyl)-O—CH₂—C—CH;
R⁰ is, at each occurrence, independently selected from C₁-C₆ alkyl, C₁-C₆ haloalkyl, C₂-C₆ alkenyl, C₂-C₆ alkynyl, substituted or unsubstituted cycloalkyl, substituted or unsubstituted heterocyclyl, substituted or unsubstituted aryl, and substituted or unsubstituted heteroaryl;
R¹ and R^{1′} are, at each occurrence, independently selected from H, hydroxyl, C₁-C₆ alkyl, C₁-C₆ haloalkyl, C₁-C₂₀ heteroalkyl, C₁-C₁₀ heteroalkyl-NH₂, C₂-C₆ alkenyl, C₂-C₆ alkynyl, substituted or unsubstituted cycloalkyl, substituted or unsubstituted heterocyclyl, substituted or unsubstituted aryl, substituted or unsubstituted arylalkyl, and substituted or unsubstituted heteroaryl, benzyl, —C(═NH)NH₂, —CH₂CO₂R⁰, —C(H)(CHₐ(COOH))₂, —CH₂C(O)NHCH₂CO₂H, —CH₂(CH₂)ₐOH, —CH₂CH₂NHC(O)R³, —(CH₂)ₐC(O)NH(CH₂)ₐC(O)OR³, —(CH₂)ₐ—CN, spermine, —(CH₂)ₐNH₂, —C(O)R³, —(CH₂)ₐC(O)OR³, —(CH₂)ₐOR³, —C(H)((CH₂)ₐOH)_{a′}, —CHCH(OH)(CH₂)ₐOH, —(CH₂)ₐOH, C₁-C₆—C(O)OH, —(CH₂)ₐ-heterocycle which may be substituted or unsubstituted, 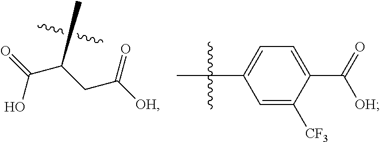
and
wherein R¹ and R^{1′} together form a substituted or unsubstituted heterocyclic ring, including, but not limited to, azetidine, pyrrolidine, piperidine, piperazine, morpholine, 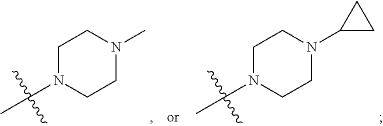
R² is, at each occurrence, independently selected from C₂-C₆ alkyl, C₁-C₆ haloalkyl, C₁-C₆ heteroalkyl, or substituted or unsubstituted cycloalkyl, substituted or unsubstituted heterocyclyl, substituted or unsubstituted aryl, substituted or unsubstituted heteroaryl, and substituted or unsubstituted haloalkoxy;
R³ is, at each occurrence, independently selected from H, C₁-C₆ alkyl, C₁-C₆ haloalkyl, C₁-C₆ heteroalkyl, —C₁-C₆—OH, —C₁-C₆—C(O)OH, or substituted or unsubstituted cycloalkyl, substituted or unsubstituted heterocyclyl, substituted or unsubstituted aryl, and substituted or unsubstituted heteroaryl, substituted or unsubstituted haloalkoxy, and guanidine;
R⁴ is, at each occurrence, independently selected from one or more heteroatom interrupted alkylene, wherein the heteroatom is O, S, NH, or a combination thereof,
Ar2 is, at each occurrence, independently selected from (i) a substituted or unsubstituted 5- and 6-membered monocyclic aromatic or heteroaromatic ring; (ii) a substituted or unsubstituted 9- and 10-membered fused bicyclic rings comprising two monocyclic rings together, where at least one of the two monocyclic rings is an aromatic or a heteroaromatic ring; and (iii) a substituted or unsubstituted 13- and 14-membered fused tricyclic rings comprising three monocyclic rings together, where at least one of the three monocyclic rings is an aromatic or a heteroaromatic ring;
wherein each Ar2 is independently substituted with G¹, G², G³, G⁴ and G⁵, wherein:
when Ar2 is monosubstituted, G¹ is, at each occurrence, independently selected from oxo, C₁-C₆ alkyl, C₁-C₆ haloalkyl, -E-O—R³, -E-C(R¹)(R^{1′})(R³), —NH₂, —NO₂, —SO₃R³, —SO₃O⁻, —SO₃N(H)(R′), -E-C(O)R³, -E-CO₂H, —B(OH)₂, —C(O)NR¹R^{1′}, -E-PO(OR¹)₂, and aryl substituted with G², G³, G⁴ and G⁵; and
G², G³, G⁴ and G⁵ are, at each occurrence, independently selected from absent or selected from the groups comprising, halogen, —CN, —NO₂, —SO₃R³, C₁-C₆ alkyl, C₁-C₆ haloalkyl, -E-O—R³, -E-(CH₂)ₐC(O)R³, -E-CO₂H, -E-CHO, -E-C(O)R³, -E-C(O)NH(OH), -E-C(O)NHR¹, -E-C(O)N(H)C(H)(R¹)(R^{1′}), -E-C(O)N(R¹)((CH₂)ₐP(O)(O)OH)₂, -E-C(O)N(R¹)((CH₂)ₐSO₃H), -E-C(O)NR¹R^{1′}, -E-NR¹R^{1′}, -E-OR², —C(O)-heterocycloalkyl (where the heterocycloalkyl is substituted or unsubstituted), —C(O)—N(H)—CH(COOH)((CH₂)ₐ-heteroaryl) (where heteroaryl is substituted or unsubstituted), wherein E is, at each occurrence, independently selected from a direct bond, and C₁-C₆ alkylene;
wherein Z is not a triazole.

2. The compound of claim 1, wherein Z includes two heteroatoms.

3. The compound of claim 1, wherein Z is a diazole.

4. The compound of claim 3, wherein the diazole is selected from the group consisting of imidazole, pyrazole, pyrimidine, pyridazine, and pyrazine.

5. The compound of claim 1, wherein Z includes three heteroatoms, four heteroatoms, or is selected from the group consisting of pyrrole, thiophene, thiazole, isoxazole, and tetrazole.

6. The compound of claim 1, wherein Ar1 is selected from the group consisting of phenyl, pyridine, furan, carbazole, naphthyl, pyridazine, thiophene, pyrrole, dibenzofuran, and naphthyridine.

7. The compound of claim 1, wherein each substituent of Ar1 is independently selected from —C(O)—CH₃, —C(O)—NH₂, —C(O)O—CH₂CH₃, —CF₃, —C(O)O-t-butyl, —C(O)—N(CH₃)₂, —C(O)—N(H)CH₃, —C(O)—N(H)CH₂CH₃, —C(O)—N(H)(cycloalkyl), —C(O)—N(H)(CH₂)₄, —C(O)—N(CH₂CH₃)₂, —C(O)—N(H)(t-butyl), —C(O)—N(H)(C₆-cycloalkyl), —C(O)—N(H)(CH₂)₃, —C(O)—N(H)(C₅-cycloalkyl), —C(O)—N(H)(C₄-cycloalkyl), —C(O)—N(H)(phenyl), —C(O)—N(H)(CH₂CH₂)N(H)C(O)—CH₃, —C(O)—N(H)C(NH)(NH₂), —C(O)—N(CH₂CH₂CH₃)₂, —C(O)—N—(C(H)(CH₃)(CH₂CH₃))₂, —C(O)—N(CH₂CH₂CH₂CH₃)₂, —C(O)—N(H)(benzyl), —C(O)—N(H)—CH₂CH₂OH, —C(O)—S—CH₂CH₃, —C(O)N(CH₂)₂, —N(H)S(O)(O)-phenyl, —C(O)N(H)CH₂CH₂CF₃, —C(O)N(H)CH₃, —C(O)OCH₂CH₃, —C(O)—N(H)—CH₂CH₂O—CH₂CH₂O—CH₃, —C(O)—N(H)—(CH₂CH₂O)₈—CH₃, —C(O)N(H)—CH₂—CH₂—CH₂—N(H)—CH₂—CH₂—CH₂—N(H)—CH₂—CH₂—CH₂—NH₂, —C(O)N(H)—CH₂—CH₂—NH₂, —C(O)N(H)C(O)CH₃, —C(O)N(H)—CH₂—CH₃, —S(O)(O)N(H)—CH₂—CH₃, —S(O)(O)N—(CH₂—CH₃)₂, —S(O)(O)N(H)—(C3-cycloalkyl), —S(O)(O)N(H)—(CH₂CH₂)OH, —S(O)(O)OH, —C(O)N(H)C(H)(CH₂OH)₂, —C(O)N(CH₂CH₂OH)₂, —C(O)N(H)CH₂CH₂═CH₂, —C(O)CH₂N(CH₃)(CH₂)(COOH), —S(O)(O)(CH₂CH₃), —S(O)(O)N(H)CH₂—CH(OH)(CH₂OH), —S(O)(O)—NH₂, and —S(O)(O)N(H)CH₂—C(O)(O-t-butyl).

8. The compound of claim 1, wherein Ar1 is substituted with at least one —SO₂—N(H)(R¹) group.

9. The compound of claim 8, wherein R¹ is a C₁-C₆ alkyl.

10. The compound of claim 9, wherein the C₁-C₆ alkyl is methyl or ethyl.

11. The compound of claim 1, wherein Ar2 is selected from the group consisting of benzene, pyridine, pyridazine, pyrimidine and pyrazine.

12. The compound of claim 1, wherein each Ar2 is substituted with one or more moieties selected from the group consisting of —NH₂, —C(O)OH, —OH, —OCH₃, —C(O)NHOH, —C(O)NH₂, —(CH₂)₃C(O)OH, —CF₃, —Cl, —P(O)(OH)₂, —F, —C(O)N(H)CH₂CH₃, —C(O)N(H)CH₂C(O)OCH₃, —C(O)N(H)CH₂CF₂CF₂CF₃, —C(O)N(H)CH₂C(O)OH, —C(O)N(H)C(H)(C(O)OOH)CH₂C(O)OH, —C(O)N(H)CH₂C(O)N(H)CH₂C(O)OH, —SO₃H, —NO₂, —C(O)N(H)CH₂PO₃H₂, —C(O)N(H)CH₂3O₃H, —B(OH)₂, —C(OH)(CF₃)(CF₃), —C(O)-5-membered heterocycloalkyl-C(O)OH, —C(O)N(H)C(H)(C(O)OH)CH₂-imidazole, —S(O)(O)N(H)—CH₂COOH, —C(O)N(H)CH(C(O)OH)(CH₂CH₂C(O)OH), —C(O)N(H)CH₂CH₂SO₃H, —SO₂O⁻, —C(O)N(H)-imidazole-C(O)OH, and —C(O)N(CH₃)₂.

13. The compound of claim 1, wherein each Ar2 is substituted with at least one —SO₃H moiety or at least one —SO₃⁻ moiety.

14. The compound of claim 1, wherein each Ar2 is substituted with at least two —SO₃H moieties or at least two —SO₃⁻ moieties.

15. The compound of claim 1, wherein each Ar2 is substituted with at least two moieties, wherein a first of the at least two moieties comprises one of a —SO₃H moiety or a —SO₃⁻ moiety.

16. The compound of claim 15, wherein a second of the at least two moieties is selected from the group consisting of —NO₂, —CF₃, —F, —Cl, —I, methyl, ethyl, propyl, and butyl.

17. The compound of claim 1, wherein the compound has any one of Formulas (IA) or (IB):

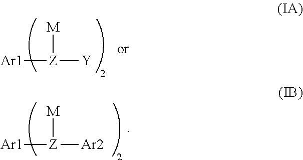

18. The compound of claim 1, wherein the compound has the formula:

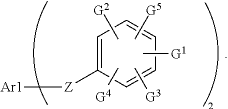

19. The compound of claim 1, wherein the compound has any one of the formulas:

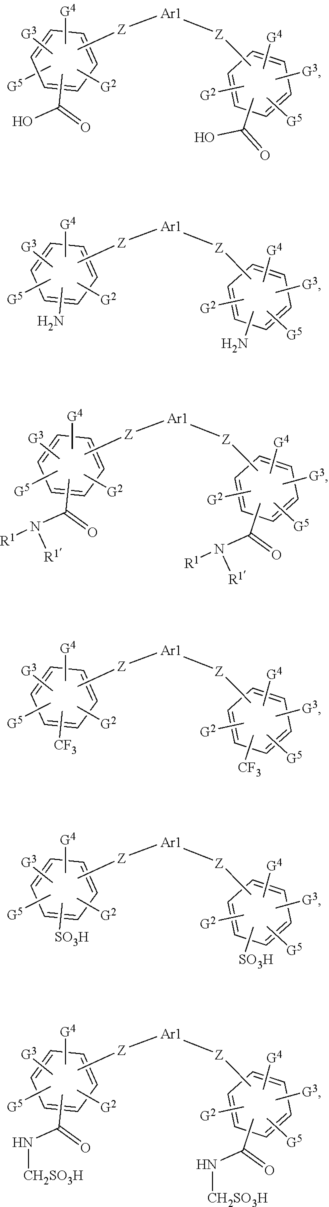

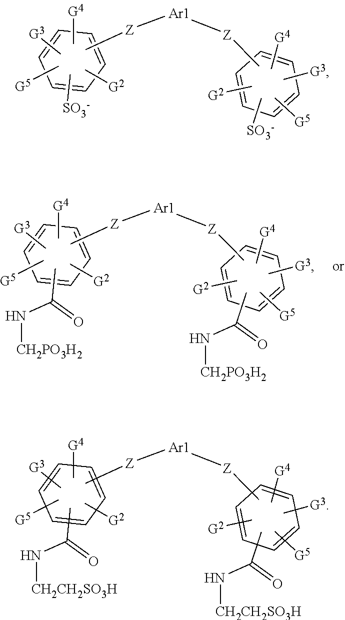

20. The compound of claim 1, wherein the compound has any one of the formulas: 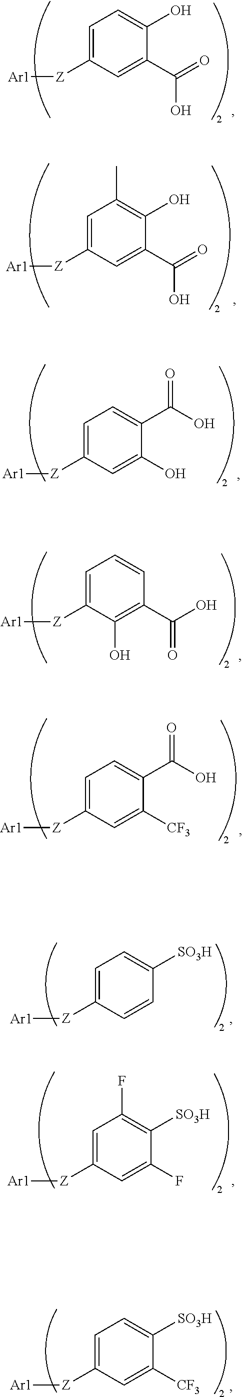 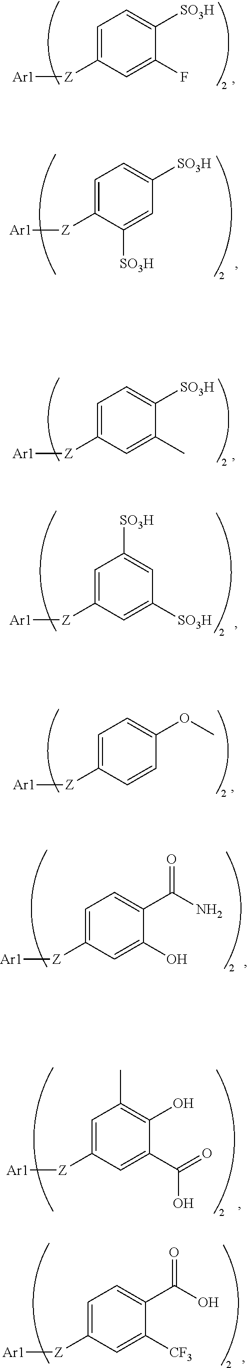 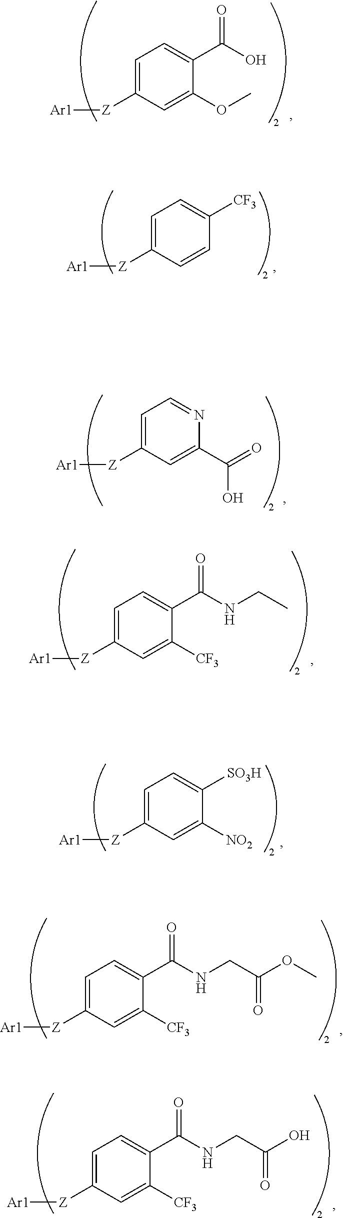 [chemical structure]
